(19) United States
(12) Patent Application Publication
(10) Pub. No.: US 2024/0376056 A1
(43) Pub. Date: Nov. 14, 2024

(54) PHENYL ACETAMIDE BASED IL-17A MODULATORS AND USES THEREOF

(71) Applicant: DiCE Alpha, Inc., South San Francisco, CA (US)

(72) Inventors: Maureen REILLY, Emerald Hills, CA (US); Paul R. FATHEREE, San Francisco, CA (US); Claudio AQUINO, Newark, CA (US); Michael FREIDBERG, South San Francisco, CA (US); Timothy J. CHURCH, San Mateo, CA (US)

(21) Appl. No.: 18/572,124

(22) PCT Filed: Jul. 8, 2022
(86) PCT No.: PCT/US2022/036569

§ 371 Date: Dec. 19, 2023

Related U.S. Application Data
(60) Provisional application No. 63/257,896, filed on Oct. 20, 2021.
(60) Provisional application No. 63/220,404, filed on Jul. 9, 2021.

(51) Int. Cl. C07D 231/14; A61K 31/415; A61K 31/4155; A61K 31/42; A61K 31/422; A61K 31/4245; A61K 31/44; A61K 31/4439; A61K 31/454; A61K 31/496; A61K 31/4985; A61K 31/5377; A61K 31/553; C07D 213/82; C07D 261/18; C07D 271/08; C07D 401/06; C07D 401/12; C07D 403/06; C07D 403/12; C07D 405/12; C07D 413/06; C07D 413/12; C07D 487/04

(52) U.S. Cl. CPC C07D 231/14; A61K 31/415; A61K 31/4155; A61K 31/42; A61K 31/422; A61K 31/4245; A61K 31/44; A61K 31/4439; A61K 31/454; A61K 31/496; A61K 31/4985; A61K 31/5377; A61K 31/553; C07D 213/82; C07D 261/18; C07D 271/08; C07D 401/06; C07D 401/12; C07D 403/06; C07D 403/12; C07D 405/12; C07D 413/06; C07D 413/12; C07D 487/04

(57) ABSTRACT

The disclosure herein provides phenyl acetamide based compounds of Formula (I), (I-a), (II), and pharmaceutical compositions thereof, for the modulation of IL-17A, useful in the treatment of inflammatory conditions such as psoriasis.

### CROSS-REFERENCE

This application is a 371 U.S. National Stage of International Application No. PCT/US2022/036569, filed Jul. 8, 2022, which claims the benefit of U.S. Provisional Patent Application No. 63/220,404 filed on Jul. 9, 2021, and U.S. Provisional Patent Application No. 63/257,896, filed on Oct. 20, 2021, the entire contents of each of which are incorporated herein by reference.

### BACKGROUND OF THE INVENTION

The IL-17 family consists of six cytokines (IL-17A through IL-17F). Interleukin-17A (IL-17A), is an established pro-inflammatory cytokine, which is involved in the induction of IL-6, IL-8, G-CSF, TNF-α, IL-1β, PGE2, and IFN-γ, as well as numerous chemokines and other effectors. IL-17A can form homodimers or heterodimers with its family member, IL-17F and can bind to both IL-17 receptors, IL-17 RA and IL-17 RC, in order to mediate signaling. IL-17A is a major pathological cytokine expressed by Th17 cells, which are involved in the pathology of inflammation and autoimmunity, and also CD8+ T cells, 75 cells, NK cells, NKT cells, macrophages and dendritic cells. Additionally. IL-17A and Th17 are necessary for defense against various microbes despite their involvement in inflammation and autoimmune disorders. Further, IL-17A can act in cooperation with other inflammatory cytokines such as TNF-α, IFN-γ, and IL-1β to mediate pro-inflammatory effects.

To date, there are a few biologics (Secukinumab and Ixekizumab) that have been approved to modulate IL-17A for the treatment of inflammatory diseases, such as psoriasis, ankylosing spondylitis, and psoriatic arthritis. These treatments require injection to a patient as they are not readily absorbed by the gut when orally ingested. Further, these approved biologic treatments have a high cost of entry for patients, limiting the availability to the patient population in need thereof.

There are a few small molecule modulators of IL-17A that have been approved for oral administration. However, while these have the convenience of oral administration and a lower cost of entry for patients, they lack the efficacy of approved biologics. Therefore, there exists a need for the development of potent small molecule IL-17A modulators for the treatment of inflammatory diseases and other associated disorders.

### SUMMARY OF THE INVENTION

In certain aspects, the present disclosure provides a compound represented by the structure of Formula (I):

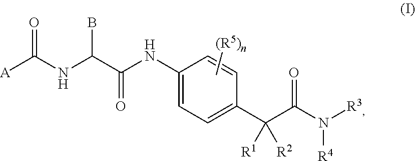

or a pharmaceutically acceptable salt thereof, wherein:
A is selected from a 5- to 6-membered heterocycle optionally substituted with one more substituents independently selected from (i), (ii), and (iii):
   (i) halogen, —OR¹¹, —SR¹¹, —N(R¹¹)₂, —C(O)R¹¹, C(O)OR¹¹, —OC(O)R¹¹, —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹¹)₂, —N(R¹¹)S(O)₂(R¹¹), —S(O)R¹¹, —S(O)₂R¹¹, —S(O)₂N(R¹¹)₂, —NO₂, and —CN;
   (ii) C₁₋₁₀ alkyl optionally substituted with one or more substituents independently selected from:
      halogen, —OR¹¹, —SR¹¹, —N(R¹¹)₂, —C(O)R¹¹, C(O)OR¹¹, —OC(O)R¹¹, —OC(O)N(R¹¹)₂, —C(O)N(R>)₂, —N(R¹¹)C(O)R¹¹, —N(R¹¹)C(O)OR¹¹, —N(R¹¹)C(O)N(R¹¹), —N(R¹¹)S(O)₂(R¹¹), —S(O)R¹¹, —S(O)₂R¹¹, —S(O)₂N(R¹¹)₂, —NO₂. ═O, —CN; and
      C₃₋₁₀ carbocycle and 3- to 10-membered heterocycle each of which is optionally substituted with one or more substituents selected from: halogen, —OR¹¹, —SR¹¹, —N(R¹¹)₂, —C(O)R¹¹, —C(O)OR¹¹, —OC(O)R¹¹, —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹¹)₂, —N(R¹¹)₁)S(O)₂(R¹¹), —S(O)R¹¹, —S(O)₂R¹¹, —S(O)₂N(R¹¹)₂, —NO₂, ═O, and —CN; and
   (iii) 3- to 10-membered heterocycle optionally substituted with one or more substituents independently selected from:
   halogen, —OR¹¹, —SR¹¹, —N(R¹¹)₂, —C(O)R¹¹, —C(O)OR¹¹, —OC(O)R¹¹, —OC(O)N(R¹¹)₂. —C(O)N(R¹¹)₂, —N(R¹¹)C(O)R¹¹, —N(R¹¹)C(O)OR¹¹, —N(R¹¹)C(O)N(R¹¹)₂, —N(R¹¹)S(O)₂(R¹¹), —S(O)R¹¹, —S(O)₂R¹¹, —S(O)₂N(R¹¹)₂, —NO₂, ═O, —CN; and
      C₁₋₆ alkyl optionally substituted with one or more substituents independently selected from halogen, —OR¹¹, —SR¹², —N(R¹¹)₂, —C(O)R¹¹, —C(O)OR¹¹, —OC(O)R¹¹, —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹¹)₂, —N(R¹¹)S(O)₂(R¹¹), —S(O)R¹¹, —S(O)₂R¹¹, —S(O₂)(R¹¹)₂, —NO₂, ═O, and —CN;
B is selected from —CH(R^{A})R^{B}) and C₃₋₁₀ carbocycle, wherein C₃₋₁₀ carbocycle is optionally substituted with one or more substituents independently selected from:
   halogen, —OR¹², —SR¹², —N(R¹²)₂, —C(O)R¹¹, —C(O)OR², —OC(O)R¹², —OC(O)N(R¹¹)₂, —C(O)N(R¹¹)₂, —N(R¹²)C(O)R¹², —N(R¹²)C(O)OR¹², —N(R²)C(O)N(R¹²)₂, —N(R¹²)S(O)₂(R¹¹), —S(O)R², —S(O)₂R¹², —S(O)₂N(R²)₂, —NO₂, ═O, and —CN; and
   C₁₋₆ alkyl optionally substituted with one or more substituents independently selected from halogen, —OR¹², —SR¹², —N(R¹²)₂, —C(O)R¹², —C(O)OR¹², —OC(O)R¹², —OC(O)N(R¹²)₂, —C(O)N(R¹²)₂, —N(R¹²)C(O)R¹², —N(R¹²)C(O)OR¹², —N(R¹²)C(O)N(R¹²)₂, —N(R¹²)S(Oh(R²), —S(O)R¹², —S(O)₂R2, —S(O)₂N(R¹²)₂, —NO₂, ═O, and —CN;
R^{A} and R^{B} are each independently selected from:
   halogen, —OR¹², —SR¹¹, —N(R¹³)₂, —C(O)R³, —C(O)OR³, —OC(O)R¹³, —OC(O)N(R¹¹)₂, —C(O)N(R¹³)₂, —N(R¹³)C(O)R¹³, —N(R¹¹)C(O)OR¹³, —N(R¹)C(O)N(R¹³)₂, —N(R¹³)S(O)₂(R¹³), —S(O)R¹¹, —S(O)₂R¹², —S(O)₂N(R¹¹)_{D})₂, —NO₂, and —CN;
   C₁₋₆ alkyl optionally substituted with one or more substituents independently selected from halogen, —OR¹², —SR¹¹, —N(R¹¹)₂, —C(O)R¹³, —C(O)OR¹³, —OC(O)R¹¹, —OC(O)N(R¹¹)₂, —C(O)N(R¹¹)₂, —N(R¹¹)C(O)R¹¹, —N(R^{1D})C(O)OR^{D}, —N(R¹¹)C(O)N(R¹¹)₂, —N(R¹¹)₂S(O)₂(R¹¹), —S(O)R¹¹, —S(O)₂R¹³, —S(O)₂N(R¹¹)₂, —NO₂, and —CN; and
C₃₋₁₀ carbocycle optionally substituted with one or more substituents independently selected from halogen, —OR¹³, —SR¹³, —N(R¹³)₂, —C(O)R¹¹, —C(O)OR¹¹, —OC(O)R¹³, —OC(O)N(R¹¹)₂, —C(O)N(R¹³)₂, —N(R¹³)C(O)R¹¹, —N(R¹¹)C(O)OR¹¹, —N(R¹¹)C(O)N(R¹¹)₂, —N(R¹³)S(Oh(R¹¹), —S(O)R¹¹, —S(O)₂R¹¹, —S(O)₂N(R¹¹)₂, —NO₂, and —CN;
R¹ and R² are each independently selected from (iv), (v), (vi) and (vii):
   (iv) hydrogen;
   (v) halogen, —OR¹⁴, —SR¹⁴, —N(R¹⁴)₂, —C(O)R¹², —C(O)OR¹⁴, —OC(O)R¹⁴, —OC(O)N(R¹¹)₂, —C(O)N(R¹⁴)₂, —N(R¹⁴)C(O)R¹⁴, —N(R¹⁴)C(O)OR¹⁴, —N(R¹⁴)C(O)N(R¹⁴)₂, —N(R¹⁴)S(O)₂(R¹⁴), —S(O)R¹⁴, —S(O)₂R4, —S(O)₂N(R¹⁴)₂, —NO₂, and —CN; and
   (vi) C₁₋₆ alkyl optionally substituted with one or more substituents independently selected from:
      halogen, —OR¹⁴, —SR¹⁴, —N(R¹⁴)₂, —C(O)R¹⁴, —C(O)OR¹⁴, —OC(O)R¹⁴, —OC(O)N(R¹⁴), —C(O)N(R¹⁴)₂, —N(R¹⁴)C(O)R¹⁴, —N(R¹⁴)C(O)OR¹⁴, —N(R¹⁴)C(O)N(R¹⁴)₂, —N(R¹⁴)S(O)₂(R¹⁴), —S(O)R¹⁴, —S(O)₂R¹², —S(O)₂N(R¹¹)₂, —NO₂, and —CN; and
      3- to 10-membered heterocycle optionally substituted with one or more substituents independently selected from halogen, —OR^{14B}, —SR^{14B}, —N(R)^{14B})₂, —C(O)R^{14B}, —C(O)OR^{14B}, —OC(O)R^{14B}, —OC(O)N(R^{14B})₂, —C(O)N(R¹⁴)₂, —N(R^{14B})C(O)R^{14B}, —N(R^{14B})C(O)OR^{14B}—N(R^{14B})C(O)N(R^{14B})₂, —N(R^{14B})S(O)(R^{14B}), —S(O)R^{14B}, —S(O)₂R^{14B}, —S(O)₂N(R^{14B})₂, —NO₂, and —CN; and
   (vii) 3- to 10-membered heterocycle optionally substituted with one or more substituents independently selected from:
      halogen, —OR^{14B}, —SR^{14B}, —N(R^{14B})₂, —C(O)R^{14B}, —C(O)OR^{14B}, —OC(O)R^{14B}, —OC(O)N(R^{14B})₂, —C(O)N(R^{14B})₂, —N(R^{14B})C(O)R^{14B}, —N(R^{14B})C(O)OR¹⁴, —N(R^{14B})C(O)N(R^{14B})₂, —N(R^{14B})S(O)₂(R^{14B}), —S(O)R^{14B}, —S(O)₂R^{14B}, —S(O)₂N(R^{14B})₂, —NO₂, and —CN
R³ and R⁴ are each independently selected from (a), (b), (c), and (d):
   (a) hydrogen;
   (b) C₁ alkyl optionally substituted with one or more substituents independently selected from:
      halogen, —OR¹¹, —SR¹¹, —N(R¹¹)₂, —C(O)R¹¹, —C(O)NH-₂, —C(O)—O—C₁.alkyl, —OC(O)R¹¹, —OC(O)N(R¹¹), —N(R¹¹)C(O)R¹¹, —N(R¹¹)C(O)OR¹⁵, —N(R¹¹)C(O)N(R¹¹)₂, —N(R¹⁵)S(O)₂(R¹⁵), —S(O)R¹⁵, —S(O)₂R¹¹, —S(O)₂N(R¹⁵), —NO₂, —CN;
      C₃₋₁₀ carbocycle and 3- to 10-membered heterocycle any of which is optionally substituted with one or more substituents independently selected from halogen, C₁₋₆ haloalkyl, C₁₋₆ alkoxy, —OR¹⁵, —SR¹¹, —N(R¹¹)₂, —C(O)R¹¹, —C(O)OR¹¹, —OC(O)R¹¹, —OC(O)N(R¹⁵)₂, —C(O)N(R¹⁵)₂, —N(R¹⁵)C(O)R¹¹, —N(R¹¹)C(O)OR₁₅, —N(R¹¹)C(O)N(R¹¹)₂, —N(R¹¹)₅)S(O)₂(R¹¹), —S(O)R¹¹, —S(O)₂R¹⁵, —S(O)₂N(R¹⁵)₂, —NO₂, and —CN;
   (c) C₂₋₆ alkyl optionally substituted with one or more substituents independently selected from:
      halogen, —OR¹¹, —SR¹¹, —N(R¹¹)₂, —C(O)R¹⁵, —C(O)—O—C₁₋₆.alkyl, —OC(O)R¹¹, —OC(O)N(R¹¹)₂, —C(O)N(R¹⁵)₂, —N(R¹⁵)₅)C(O)R¹¹, —N(R¹⁵)C(O)OR¹⁵, —N(R¹¹)C(O)N(R¹⁵)₂, —N(R¹⁵)S(O)₂(R¹⁵), —S(O)R¹¹, —S(O)₂R¹⁵, —S(O)₂N(R¹¹)₂, —NO₂, and —CN;
      C₃₋₁₀ carbocycle and 3- to 10-membered heterocycle any of which is optionally substituted with one or more substituents independently selected from halogen, C₁₋₆haloalkyl, C₁₋₆ alkoxy, —OR¹¹)₅, —SR¹⁵, —N(R¹⁴)₂, —C(O)R¹¹, —C(O)OR³, —OC(O)R¹¹, —OC(O)N(R¹¹)₂, —C(O)N(R¹¹)₂, —N(R¹¹)C(O)R¹¹, —N(R¹⁵)C(O)OR¹⁵, —N(R¹¹)C(O)N(R¹¹)₂, —N(R¹¹)S(O)₂(R¹¹), —S(O)R¹⁴, —S(O)₂R¹⁵, —S(O)₂N(R¹⁵)₂, —NO₂, and —CN; and
   (d) C₃₋₁₀ carbocycle and 3- to 10-membered heterocycle any of which is optionally substituted with one or more substituents independently selected from:
      halogen, —OR¹¹, —SR¹¹, —N(R¹¹)₂, —C(O)R¹¹, —C(O)OR¹¹, —OC(O)R¹¹, —OC(O)N(R¹⁵)₂, —C(O)N(R¹¹)₂, —N(R¹¹)C(O)R¹¹, —N(R¹¹)C(O)OR¹¹, —N(R¹¹)C(O)N(R¹¹)₂, —N(R¹¹)S(O)₂(R¹¹), —S(O)R¹⁴, —S(O)₂R¹¹, —S(O)₂N(R¹¹)₂, —NO₂, and —CN; and
      C₁₋₆ alkyl optionally substituted with one or more substituents independently selected from halogen, —OR¹¹, —SR^{11B}, —N(R¹¹)₂, —C(O)R¹¹, —C(O)OR¹¹, —OC(O)R¹¹, —OC(O)N(R¹¹)₂, —C(O)N(R¹⁴)₂, —N(R¹⁴)C(O)R¹⁴, —N(R¹⁴)C(O)OR¹⁵, —N(R¹¹)C(O)N(R¹⁴)₂, —N(R¹⁵)S(O)₂(R¹⁵), —S(O)R¹⁵, —S(O)₂R¹¹)₃, —S(O)₂N(R¹⁵)₂, —NO₂, and —CN; or
R³ and R⁴ can come together to form a 4- to 12-membered heterocycle optionally substituted with one or more substituents independently selected from:
   halogen, —OR¹⁶, —SR¹⁶, —N(R¹⁴)₂, —C(O)R⁶, —C(O)OR¹⁶, —OC(O)R¹⁶, —OC(O)N(R¹⁶), —C(O)N(R¹⁶)₂, —N(R¹⁶)C(O)R¹⁶, —N(R¹⁶)C(O)OR¹⁶, —N(R¹⁶)C(O)N(R¹⁶)₂, —N(R¹⁶)S(O)₂(R¹⁶), —S(O)R¹⁶, —S(O)₂R¹⁵, —S(O)₂N(R¹⁶)₂, —NO₂. =0, and —CN; and
      C₁₋₆ alkyl optionally substituted with one or more substituents independently selected from halogen, —OR¹⁶, —SR¹⁶, —N(R¹⁶)₂, —C(O)R¹⁶, —C(O)OR¹⁶, —OC(O)R¹⁵, —OC(O)N(R¹⁶)₂, —C(O)N(R¹⁶)₂, —N(R¹⁶)C(O)R⁶, —N(R¹⁶)C(O)OR¹⁶, —N(R¹⁶)C(O)N(R¹⁶)₂, —N(R¹⁶)S(O)₂(R¹⁶), —S(O)R¹⁶, —S(O)₂R¹⁶, —S(O)₂N(R¹⁶)₂, —NO₂, ═O, and —CN;
R⁵ is selected from:
   halogen, —OR¹⁷, —SR¹⁷, —N(R¹⁷)₂, —C(O)R¹¹, —C(O)OR¹⁷, —OC(O)R¹⁷, —OC(O)N(R¹¹)₂, —C(O)N(R¹⁷)₂, —N(R¹⁷)C(O)R¹⁷, —N(R¹⁷)C(O)OR¹¹, —N(R¹¹)C(O)N(R¹⁷)₂, —N(R¹⁷)S(O)₂(R¹⁷), —S(O)R¹⁷, —S(O)R¹⁷, —S(O)₂N(R¹⁷)₂, —NO₂, and —CN;
   C₁₋₆ alkyl and C₂₋₆ alkenyl each of which is optionally substituted with one or more substituents independently selected from halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹¹, —N(R¹⁷)C(O)N(R¹⁷)₂, —N(R¹⁷)S(O)₂(R¹⁷), —S(O)R¹⁷, —S(O)₂R¹⁷, —S(O)₂N(R¹⁷)₂, —NO₂=0, and —CN; and
   C₃₋₁₀ carbocycle optionally substituted with one or more substituents independently selected from halogen, —OR^{11B}, —SR¹⁷¹, —N(R¹¹)₇h)₂, —C(O)R¹¹)_{7B}, —C(O)OR^{17A}, —OC(O)R^{17B}, —OC(O)N(R¹⁷)₂, —C(O)N(R¹⁴)₂, —N(R¹⁴)C(O)R¹⁴, —N(R¹⁷)C(O)OR¹⁷B, —N(R^{17B})C(O)N(R^{17B})₂, —N(R^{17B})S(O)₂(R^{17B}), —S(O)R^{17B}, —S(O)₂R^{17B}, —S(O)₂N(R^{17B})₂, —NO₂, and —CN;
R¹¹, R¹², R¹³, R¹⁴, R^{14B}. R^{11B}, R¹⁶, R¹⁷, and RIm are independently selected at each occurrence from (I), (II) and (III):
   (I) hydrogen;
   (II) C₁₋₆ alkyl optionally substituted with one or more substituents independently selected from. halogen, —OH, —O—C₁-C₆ alkyl, —O—C₁-C₆haloalkyl —NH₂, —NO₂, ═O, —CN, C₃₋₁₀ carbocycle and 3- to 10-membered heterocycle, wherein the C₃₋₁₀ carbocycle and 3- to 10-membered heterocycle are optionally substituted with one or more substituents selected from: halogen, —OH, C₁-C₆ haloalkyl, —O—C₁-C₆ alkyl, —O—C₁-C₆ haloalkyl —NH₂, —NO₂, ═O, and —CN; and
   (III) C₃₋₁₀ carbocycle and 3- to 10-membered heterocycle. each of which is optionally substituted with one or more substituents independently selected from: halogen, —OH, C₁-C₆ haloalkyl, —O—C₁-C₆ alkyl, —O—C₁-C₆ haloalkyl —NH₂, —NO₂, ═O, and —CN; and C₁₋₆ alkyl optionally substituted with one or more substituents independently selected from halogen, —OH, —O—C₁-C₆ alkyl, —O—C₁-C₆ aloalkyl —NH₂, —NO₂, ═O, and —CN; and
      n is selected from 0, 1, 2, 3, and 4,
      wherein n is selected from 1, 2, 3, and 4; or
      at least one of R¹ or R² is hydrogen.

In certain embodiments, the disclosure provides a pharmaceutical composition comprising pharmaceutically acceptable excipient and a compound or salt of Formula (I) or (I-a).

In certain embodiments, the disclosure provides a method of modulating IL-17 A in a subject in need thereof, comprising administering to the subject a compound or salt of Formula (I) or (I-a). or a pharmaceutical composition thereof.

In certain embodiments, the disclosure provides a method treating an inflammatory disease or condition comprising administering to the subject a compound or salt of Formula (I) or (I-a), or a pharmaceutical composition thereof. In some embodiments, the inflammatory disease or condition is selected from plaque psoriasis, guttate psoriasis, inverse psoriasis, pustular psoriasis. erythrodermic psoriasis, psoriatic arthritis, ankylosing spondylitis, hidradenitis suppurativa, rheumatoid arthritis, Palmoplantar Psoriasis, Spondyloarthritis, and Non-infectious Uveitis.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Definitions

Unless defined otherwise, all technical and scientific terms used herein have the same meaning as is commonly understood by one of skill in the art to which this invention belongs. All patents and publications referred to herein are incorporated by reference.

As used in the specification and claims, the singular form “a”, “an” and “the” includes plural references unless the context clearly dictates otherwise.

“Alkyl” refers to a straight or branched hydrocarbon chain monovalent radical consisting solely of carbon and hydrogen atoms, containing no unsaturation, and preferably having from one to twelve carbon atoms (i.e, C₁-C₁₂ alkyl). The alkyl is attached to the remainder of the molecule through a single bond. In certain embodiments, an alkyl comprises one to twelve carbon atoms (i.e. C₁-C₁₂ alkyl). In certain embodiments, an alkyl comprises one to eight carbon atoms (i.e, C₁-C₈ alkyl). In other embodiments, an alkyl comprises one to five carbon atoms (i.e, C₁-C₅ alkyl). In other embodiments, an alkyl comprises one to four carbon atoms (i.e, C₁-C₄ alkyl). In other embodiments, an alkyl comprises one to three carbon atoms (i.e, C₁-C₃ alkyl). In other embodiments, an alkyl comprises one to two carbon atoms (i.e. C₁-C₂ alkyl). In other embodiments, an alkyl comprises one carbon atom (i.e, C₁ alkyl). In other embodiments, an alkyl comprises five to fifteen carbon atoms (i.e, C₃-C₁₅ alkyl). In other embodiments, an alkyl comprises five to eight carbon atoms (i.e. C₅C₈ alkyl). In other embodiments, an alkyl comprises two to five carbon atoms (i.e, C₂-C₅ alkyl). In other embodiments, an alkyl comprises three to five carbon atoms (i.e, C₃-C₅ alkyl). For example, the alkyl group may be attached to the rest of the molecule by a single bind, such as, methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl), and the like.

“Alkenyl” refers to a straight or branched hydrocarbon chain radical group consisting solely of carbon and hydrogen atoms, containing at least one carbon-carbon double bond, and preferably having from two to twelve carbon atoms (i.e, C₂-C₁₂ alkenyl). In certain embodiments, an alkenyl comprises two to eight carbon atoms (i.e, C₂-C₅ alkenyl). In certain embodiments, an alkenyl comprises two to six carbon atoms (i.e, C₂-C₆ alkenyl). In other embodiments, an alkenyl comprises two to four carbon atoms (i.e, C₂-C₄ alkenyl). The alkenyl is attached to the rest of the molecule by a single bond, for example, ethenyl (i.e, vinyl), prop-1-enyl (i.e, allyl), but-1-en^{y}1, pent-1-enyl, penta-1,4-dienyl, and the like.

“Alkynyl” refers to a straight or branched hydrocarbon chain radical group consisting solely of carbon and hydrogen atoms, containing at least one carbon-carbon triple bond, and preferably having from two to twelve carbon atoms (i.e, C₂-C₁₂ alkynyl). In certain embodiments, an alkynyl comprises two to eight carbon atoms (i.e, C₂-C₅ alkynyl). In other embodiments, an alkynyl comprises two to six carbon atoms (i.e, C₂-C₆ alkynyl). In other embodiments, an alkynyl comprises two to four carbon atoms (i.e, C₂-C₄ alkynyl). The alkynyl is attached to the rest of the molecule by a single bond, for example, ethynyl, propynyl, butynyl, pentynyl, hexynyl, and the like.

“Alkylene” refers to a straight divalent hydrocarbon chain linking the rest of the molecule to a radical group, consisting solely of carbon and hydrogen, containing no unsaturation, and preferably having from one to twelve carbon atoms, for example, methylene, ethylene, propylene, butylene, and the like. The alkylene chain is attached to the rest of the molecule through a single bond and to the radical group through a single bond. The points of attachment of the alkylene chain to the rest of the molecule and to the radical group are through the terminal carbons respectively.

Alkylene chain may be optionally substituted by one or more substituents such as those substituents described herein. In certain embodiments, an alkylene comprises one to ten carbon atoms (i.e. C₁-C₁₀ alkylene). In certain embodiments, an alkylene comprises one to eight carbon atoms (i.e. C₁-C₈ alkylene). In other embodiments, an alkylene comprises one to five carbon atoms (i.e, C₁-C₃ alkylene). In other embodiments, an alkylene comprises one to four carbon atoms (i.e, C₁-C₄ alkylene). In other embodiments, an alkylene comprises one to three carbon atoms (i.e, C₁-C₃ alkylene). In other embodiments, an alkylene comprises one to two carbon atoms (i.e, C₁-C₂ alkylene). In other embodiments, an alkylene comprises one carbon atom (i.e, C₁ alkylene). In other embodiments, an alkylene comprises five to eight carbon atoms (i.e, C₅-C₈ alkylene). In other embodiments, an alkylene comprises two to five carbon atoms (i.e, C₂-C₅ alkylene). In other embodiments, an alkylene comprises three to five carbon atoms (i.e, C₃-C₅ alkylene).

“Alkenylene” refers to a straight divalent hydrocarbon chain linking the rest of the molecule to a radical group, consisting solely of carbon and hydrogen, containing at least one carbon-carbon double bond, and preferably having from two to twelve carbon atoms. The alkenylene chain is attached to the rest of the molecule through a single bond and to the radical group through a single bond. The points of attachment of the alkenylene chain to the rest of the molecule and to the radical group are through the terminal carbons respectively. Alkenylene chain may be optionally substituted by one or more substituents such as those substituents described herein. In certain embodiments, an alkenylene comprises two to ten carbon atoms (i.e. C₂-C₁₀ alkenylene). In certain embodiments, an alkenylene comprises two to eight carbon atoms (i.e, C₂-C₅ alkenylene). In other embodiments, an alkenylene comprises two to five carbon atoms (i.e, C₂-C₅ alkenylene). In other embodiments, an alkenylene comprises two to four carbon atoms (i.e. C₂-C₄ alkenylene). In other embodiments, an alkenylene comprises two to three carbon atoms (i.e. C₂-C₃ alkenylene). In other embodiments, an alkenylene comprises two carbon atom (i.e, C₂ alkenylene). In other embodiments, an alkenylene comprises five to eight carbon atoms (i.e, C₅C₈ alkenylene). In other embodiments, an alkenylene comprises three to five carbon atoms (i.e, C₃-C₅ alkenylene).

“Alkynylene” refers to a straight divalent hydrocarbon chain linking the rest of the molecule to a radical group, consisting solely of carbon and hydrogen, containing at least one carbon-carbon triple bond, and preferably having from two to twelve carbon atoms. The alkynylene chain is attached to the rest of the molecule through a single bond and to the radical group through a single bond. The points of attachment of the alkynylene chain to the rest of the molecule and to the radical group are through the terminal carbons respectively. Alkynylene chain may be optionally substituted by one or more substituents such as those substituents described herein. In certain embodiments, an alkynylene comprises two to ten carbon atoms (i.e. C₂-C₁₀ alkynylene). In certain embodiments, an alkynylene comprises two to eight carbon atoms (i.e, C₂-C₈ alkynylene). In other embodiments, an alkynylene comprises two to five carbon atoms (i.e, C₂-C₅ alkynylene). In other embodiments, an alkynylene comprises two to four carbon atoms (i.e, C₂-C₄ alkynylene). In other embodiments, an alkynylene comprises two to three carbon atoms (i.e, C₂-C₃ alkynylene). In other embodiments, an alkynylene comprises two carbon atom (i.e, C₂ alkynylene). In other embodiments, an alkynylene comprises five to eight carbon atoms (i.e. C₅-C₈ alkynylene). In other embodiments, an alkynylene comprises three to five carbon atoms (i.e. C₃-C₅ alkynylene).

The term “C_{x-y}” when used in conjunction with a chemical moiety, such as alkyl, alkenyl, or alkynyl is meant to include groups that contain from x to y carbons in the chain. For example, the term “C₁₋₆ alkyl” refers to substituted or unsubstituted saturated hydrocarbon groups, including straight-chain alkyl and branched-chain alkyl groups that contain from 1 to 6 carbons. The term —C_{x-y} alkylene- refers to a substituted or unsubstituted alkylene chain with from x to y carbons in the alkylene chain. For example, —C₁₋₆ alkylene- may be selected from methylene, ethylene, propylene, butylene, pentylene, and hexylene, any one of which is optionally substituted.

The terms “C_{x-y} alkenyl” and “C_{x-y} alkynyl” refer to unsaturated aliphatic groups analogous in length and possible substitution to the alkyls described above, but that contain at least one double or triple bond, respectively. The term —C, alkenylene- refers to a substituted or unsubstituted alkenylene chain with from x to y carbons in the alkenylene chain. For example, —C₂₋₆ alkenylene may be selected from ethenylene, propenylene, butenylene, pentenylene, and hexenylene, any one of which is optionally substituted. An alkenylene chain may have one double bond or more than one double bond in the alkenylene chain. The term —C_{x-y}-alkynylene- refers to a substituted or unsubstituted alkynylene chain with from x to y carbons in the alkynylene chain. For example, —C₂₋₆ alkynylene- may be selected from ethynylene, propynylene, butynylene, pentynylene, and heyvnylene, any one of which is optionally substituted. An alkynylene chain may have one triple bond or more than one triple bond in the alkynylene chain.

The term “carbocycle” as used herein refers to a saturated, unsaturated or aromatic ring in which each atom of the ring is carbon. Carbocycle include 3- to 10-membered monocyclic rings and 6- to 12-membered bicyclic rings. Each ring of a bicyclic carbocycle may be selected from saturated, unsaturated, and aromatic rings. Bicyclic carbocycles may be fused, bridged or spiro-ring systems. In some embodiments, the carbocycle is an aryl. In some embodiments, the carbocycle is a cycloalkyl. In some embodiments, the carbocycle is a cycloalkenyl. In an exemplary embodiment, an aromatic ring, e.g, phenyl, may be fused to a saturated or unsaturated ring, e.g, cyclohexane, cyclopentane, or cyclohexene. Any combination of saturated, unsaturated and aromatic bicyclic rings, as valence permits, are included in the definition of carbocyclic. Exemplary carbocycles include cyclopentyl, cyclohexyl, cyclohexenyl, adamantyl, phenyl, indanyl, and naphthyl. Carbocycle may be optionally substituted by one or more substituents such as those substituents described herein.

“Cycloalkyl” refers to a stable fully saturated monocyclic or polycyclic hydrocarbon radical consisting solely of carbon and hydrogen atoms, which includes fused or bridged ring systems, and preferably having from three to twelve carbon atoms (i.e, C₃₋₁₂ cycloalkyl). In certain embodiments, a cycloalkyl comprises three to ten carbon atoms (i.e. C₃₋₁₀ cycloalkyl). In other embodiments, a cycloalkyl comprises five to seven carbon atoms (i.e. C₅₋₇ cycloalkyl). The cycloalkyl may be attached to the rest of the molecule by a single bond. Examples of monocyclic cycloalkyls include, e.g, cyclopropyl, cyclobutyl, Cyclopentyl, cyclohexyl, cycloheptyl, and cyclooctyl. Polycyclic cycloalkyl radicals include, for example, adamantyl, norbomyl (i.e. bicyclo[2.2.1]heptanyl), norbomenyl, decalinyl, 7,7-dimethyl-bicyclo[2.2.1]heptanyl, and the like. Cycloalkyl may be optionally substituted by one or more substituents such as those substituents described herein.

“Cycloalkenyl” refers to a stable unsaturated non-aromatic monocyclic or polycyclic hydrocarbon radical consisting solely of carbon and hydrogen atoms, which includes fused or bridged ring systems, preferably having from three to twelve carbon atoms and comprising at least one double bond (i.e. C₃₋₁₂ cycloalkenyl). In certain embodiments, a cycloalkenyl comprises three to ten carbon atoms (i.e. C₃₋₁₀ cycloalkenyl). In other embodiments, a cycloalkenyl comprises five to seven carbon atoms (i.e, C₅₋₇ cycloalkenyl). The cycloalkenyl may be attached to the rest of the molecule by a single bond. Examples of monocyclic cycloalkenyls include, e.g, cyclopentenyl, cyclohexenyl, cycloheptenyl, and cyclooctenyl. Cycloalkenyl may be optionally substituted by one or more substituents such as those substituents described herein.

“Aryl” refers to a radical derived from an aromatic monocyclic or aromatic multicyclic hydrocarbon ring system by removing a hydrogen atom from a ring carbon atom. The aromatic monocyclic or aromatic multicyclic hydrocarbon ring system contains only hydrogen and carbon and from five to eighteen carbon atoms, where at least one of the rings in the ring system is aromatic, i.e, it contains a cyclic. delocalized (4n+2) π-electron system in accordance with the Hückel theory. The ring system from which aryl groups are derived include, but are not limited to, groups such as benzene, fluorene, indane, indene, tetralin and naphthalene. Aryl may be optionally substituted by one or more substituents such as those substituents described herein.

A “C_{x-y} carbocycle” is meant to include groups that contain from x to y carbons in a ring. For example, the term “C₃₋₆ carbocycle” can be a saturated, unsaturated or aromatic ring system that contains from 3 to 6 carbon atoms-any of which is optionally substituted as provided herein.

The term “heterocycle” as used herein refers to a saturated, unsaturated, non-aromatic or aromatic ring comprising one or more heteroatoms. Exemplary heteroatoms include N. O, Si, P, B, and S atoms. Heterocycles include 3- to 10-membered monocyclic rings and 6- to 12-membered bicyclic rings. Each ring of a bicyclic heterocycle may be selected from saturated, unsaturated, and aromatic rings. In some embodiments, the heterocycle comprises at least one heteroatom selected from oxygen, nitrogen, sulfur, or any combination thereof. In some embodiments, the heterocycle comprises at least one heteroatom selected from oxygen, nitrogen. or any combination thereof. In some embodiments, the heterocycle comprises at least one heteroatom selected from oxygen, sulfur, or any combination thereof. In some embodiments, the heterocycle comprises at least one heteroatom selected from nitrogen, sulfur, or any combination thereof. The heterocycle may be attached to the rest of the molecule through any atom of the heterocycle. valence permitting, such as a carbon or nitrogen atom of the heterocycle. In some embodiments, the heterocycle is a heteroaryl. In some embodiments, the heterocycle is a heterocycloalkyl. Exemplary heterocycles include pyrrolidinyl, pyrrolyl, imidazolyl, pyrazolyl, triazolyl, piperidinyl. pyridinyl, pyrimidinyl, pyridazinyl. pyrazinyl, thiophenyl, oxazolyl, thiazolyl. morpholinyl. indazolyl, indolyl, and quinolinyl. Heterocycle may be optionally substituted by one or more substituents such as those substituents described herein. Bicyclic heterocycles may be fused, bridged or spiro-ring systems. In an exemplary embodiment, a heterocycle, e.g, pyridyl, may be fused to a saturated or unsaturated ring, e.g, cyclohexane, cyclopentane, or cyclohexene. Heterocycle may be optionally substituted by one or more substituents such as those substituents described herein.

“Heterocycloalkyl” refers to a stable 3- to 12-membered non-aromatic ring radical that comprises two to twelve carbon atoms and at least one heteroatom wherein each heteroatom may be selected from N. O, Si, P. B. and S atoms. In some embodiments, the heterocycloalkyl comprises at least one heteroatom selected from oxygen, nitrogen, sulfur, or any combination thereof. In some embodiments, the heterocycloalkyl comprises at least one heteroatom selected from oxygen, nitrogen, or any combination thereof. In some embodiments, the heterocycloalkyl comprises at least one heteroatom selected from oxygen, sulfur, or any combination thereof. In some embodiments, the heterocycloalkyl comprises at least one heteroatom selected from nitrogen, sulfur, or any combination thereof. The heterocycloalkyl may be selected from monocyclic or bicyclic, and fused or bridged ring systems. The heteroatoms in the heterocycloalkyl radical are optionally oxidized.

One or more nitrogen atoms, if present, are optionally quatem/zed. The heterocycloalkyl radical is partially or fully saturated. The heterocycloalkyl is attached to the rest of the molecule through any atom of the heterocycloalkyl, valence permitting, such as any carbon or nitrogen atoms of the heterocycloalkyl. Examples of heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl. thiamorpholinyl, 1-oxo-thiomorpholinyl, and 1,1-dioxo-thiomorpholinyl. Heterocycloalkyl may be optionally substituted by one or more substituents such as those substituents described herein.

The term “heteroaryl” refers to a radical derived from a 3- to 12-membered aromatic ring radical that comprises one to eleven carbon atoms and at least one heteroatom wherein each heteroatom may be selected from N, O, and S. In some embodiments, the heteroaryl comprises at least one heteroatom selected from oxygen, nitrogen, sulfur, or any combination thereof. In some embodiments, the heteroaryl comprises at least one heteroatom selected from oxygen, nitrogen, or any combination thereof. In some embodiments, the heteroaryl comprises at least one heteroatom selected from oxygen, sulfur, or any combination thereof. In some embodiments, the heteroaryl comprises at least one heteroatom selected from nitrogen, sulfur, or any combination thereof. As used herein, the heteroaryl ring may be selected from monocyclic or bicyclic and fused or bridged ring systems rings wherein at least one of the rings in the ring system is aromatic, i.e, it contains a cyclic, delocalized (4n+2) π-electron system in accordance with the Hackel theory. The heteroatom(s) in the heteroaryl radical may be optionally oxidized. One or more nitrogen atoms, if present, are optionally quatem/zed. The heteroaryl may be attached to the rest of the molecule through any atom of the heteroaryl, valence permitting, such as a carbon or nitrogen atom of the heteroaryl. Heteroaryl includes aromatic single ring structures, preferably 5- to 6-membered rings, whose ring structures include at least one heteroatom, preferably one to four heteroatoms, more preferably one or two heteroatoms. Heteroaryl groups include, for example, pyrrole, furan, thiophene, imidazole, oxazole, thiazole, pyrazole, pyridine, pyrazine, pyridazine, and pyrimidine, and the like. Heteroaryl may be optionally substituted by one or more substituents such as those substituents described herein. Heteroaryl also includes polycyclic ring systems having two or more rings in which two or more atoms are common to two adjoining rings wherein at least one of the rings is heteroaromatic, e.g, the other rings can be aromatic or non-aromatic carbocyclic, or heterocyclic. Heteroaryl may be optionally substituted by one or more substituents such as those substituents described herein.

An “X-membered heterocycle” refers to the number of endocylic atoms, i.e, X, in the ring.

For example, a 5-membered heteroaryl ring or 5-membered aromatic heterocycle has 5 endocyclic atoms, e.g, triazole, oxazole, thiophene, etc.

“Alkoxy” refers to a radical bonded through an oxygen atom of the formula —O-alkyl, where alkyl is an alkyl chain as defined above.

“Halo” or “halogen” refers to halogen substituents such as bromo, chloro, fluoro and iodo substituents.

As used herein, the term “haloalkyl” or “haloalkane” refers to an alkyl radical, as defined above, that is substituted by one or more halogen radicals, for example, trifluoromethyl, dichloromethyl, bromomethyl, 2,2,2-trifluoroethyl, 1-fluoromethyl-2-fluoroethyl, and the like. In some embodiments, the alkyl part of the fluoroalkyl radical is optionally further substituted.

Examples of halogen substituted alkanes (“haloalkanes”) include halomethane (e.g, chloromethane, bromomethane, fluoromethane, iodomethane), di- and trihalomethane (e.g, trichloromethane, tribromomethane, trifluoromethane, triiodomethane), 1-haloethane, 2-haloethane, 1,2-dihaloethane, 1-halopropane, 2-halopropane, 3-halopropane, 1,2-dihalopropane, 1,3-dihalopropane, 2,3-dihalopropane, 1,2,3-trihalopropane, and any other suitable combinations of alkanes (or substituted alkanes) and halogens (e.g, Cl, Br, F, and I). When an alkyl group is substituted with more than one halogen radicals, each halogen may be independently selected for example, 1-chloro,2-fluoroethane.

The term “substituted” refers to moieties having substituents replacing a hydrogen on one or more carbons or substitutable heteroatoms, e.g, an NH or NH₂ of a compound. It will be understood that “substitution” or “substituted with” includes the implicit proviso that such substitution is in accordance with permitted valence of the substituted atom and the substituent, and that the substitution results in a stable compound, i.e, a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds.

In some embodiments. substituents may include any substituents described herein, for example: halogen, hydroxy, oxo (=O), thioxo (=S), cyano (—CN), nitro (—NO₂), imino (=N—H), oximo (=N—OH), hydrazino (=N—NH₂), —R^{b}—OR, —R^{b}—OC(O)—R¹², —R^{b}—OC(O)—OR¹², —R^{b}—OC(O)—N(R¹¹)₂, —R^{b}—N(R^{a})₂, —R^{b}—C(O)R¹², —R^{b}—C (O)OR¹², —R^{b}—C(O)N(R¹¹)₂, —R^{b}—O—R^{o}—C(O)N(R¹¹)₂, —R^{b}—N(R¹¹)C(O)OR, —R^{b}—N(R¹¹)C(O)R¹², —R^{b}—N(R^{o}) S(O)R (where t is 1 or 2), —R^{c}—S(O)R¹ (where t is 1 or 2), —R—S(O)OR¹ (where t is 1 or 2), and —R—S(O)N(R¹¹)₂ (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl any of which may be optionally substituted by alkyl, alkenyl, alkynyl, halogen, haloalkyl, haloalkenyl, haloalkynyl, oxo (=O), thioxo (=S), cyano (—CN), nitro (—NO₂), imino (=N—H), oximo(=N—OH), hydrazine(=N—NH₂), —R^{b}—OR¹², —R^{b}—OC(O)—R¹², —Re—OC(O)—OR^{a}, —Re—OC(O)—N(R¹⁴)₂, —R^{b}—N(R¹¹)₂, —R^{b}—C(O)Ra, —R^{b}—C (O)OR¹², —R^{b}—C(O)N(R¹⁴)₂, —R^{b}—O—R—C(O)N(R)₂, —R^{b}—N(R¹⁴)C(O)OR^{a}, —R^{b}—N(R¹¹)C(O)R¹², —R^{b}—N(R¹⁴) S(O)XR¹ (where t is I or 2), —R^{b}—S(O)_{H}R^{V} (where t is I or 2), —R—S(O)OR¹ (where t is I or 2) and —Re—S(O)N(R¹¹)₂ (where t is 1 or 2); wherein each R¹ is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R¹¹, valence permitting, may be optionally substituted with alkyl, alkenyl, alkynyl, halogen, haloalkyl, haloalkenyl, haloalkynyl, oxo (═O), thioxo (=S), cyano (—CN), nitro (—NO₂), imino (=N—H), oximo (=N—OH), hydrazine(═N-NH₂), —R^{b}—OR¹², —Re—OC(O)—R. —R—OC(O)—OR¹¹, —R—OC(O)—N(R¹¹), —R^{b}—N(R)₂, —R^{b}—C(O)R¹¹, —R^{b}—C (O)OR¹², —R^{b}—C(O)N(R¹¹)₂, —R^{b}—O—R^{o}—C(O)N(R¹¹)₂, —R^{b}—N(R¹¹)C(O)OR¹², —R—N(R)C(O)R¹², —R^{b}—N(R^{a}) S(O)NR¹ (where t is 1 or 2), —R^{b}—S(O)ₜR^{b} (where t is 1 or 2), —Re—S(O)ORa (where t is 1 or 2) and —R—S(O)tN(R¹¹)₂ (where t is 1 or 2); and wherein each R^{b} is independently selected from a direct bond or a straight or branched alkylene, alkenylene, or alkynylene chain, and each R¹ is a straight or branched alkylene, alkenylene or alkynylene chain. It will be understood by those skilled in the art that substituents can themselves be substituted, if appropriate.

The term “salt” or “pharmaceutically acceptable salt” refers to salts derived from a variety of organic and inorganic counter ions well known in the art. Pharmaceutically acceptable acid addition salts can be formed with inorganic acids and organic acids. Pharmaceutically acceptable base addition salts can be formed with inorganic and organic bases.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carner” as used herein means a pharmaceutically acceptable material. composition or vehicle, such as a liquid or solid filler. diluent, excipient, solvent or encapsulating material. Each carrier must be “acceptable” in the sense of being compatible with the other ingredients of the formulation and not injurious to the patient.

The terms “subject.” “individual,” and “patient” may be used interchangeably and refer to humans, the as well as non-human mammals (e.g, non-human primates, canines, equines, felines, porcines, bovines, ungulates, lagomorphs, and the like). In various embodiments, the subject can be a human (e.g, adult male, adult female, adolescent male, adolescent female, male child, female child) under the care of a physician or other health worker in a hospital, as an outpatient, or other clinical context. In certain embodiments, the subject may not be under the care or prescription of a physician or other health worker.

As used herein, the phrase “a subject in need thereof” refers to a subject, as described infra, that suffers from, or is at risk for, a pathology to be prophylactically or therapeutically treated with a compound or salt described herein.

The terms “administer”, “administered”. “administers” and “administering” are defined as providing a composition to a subject via a route known in the art, including but not limited to intravenous, intraarterial, oral, parenteral, buccal, topical, transdermal, rectal, intramuscular, subcutaneous, intraosseous, transmucosal, or intraperitoneal routes of administration. In certain embodiments, oral routes of administering a composition can be used. The terms ““administer”, “administered”, “administers” and “administering” a compound should be understood to mean providing a compound of the invention or a prodrug of a compound of the invention to the individual in need.

As used herein, “treatment” or “treating” refers to an approach for obtaining beneficial or desired results with respect to a disease, disorder, or medical condition including, but not limited to, a therapeutic benefit and/or a prophylactic benefit. In certain embodiments, treatment or treating involves administering a compound or composition disclosed herein to a subject. A therapeutic benefit may include the eradication or amelioration of the underlying disorder being treated. Also, a therapeutic benefit may be achieved with the eradication or amelioration of one or more of the physiological symptoms associated with the underlying disorder, such as observing an improvement in the subject, notwithstanding that the subject may still be afflicted with the underlying disorder. In certain embodiments, for prophylactic benefit, the compositions are administered to a subject at risk of developing a particular disease, or to a subject reporting one or more of the physiological symptoms of a disease, even though a diagnosis of this disease may not have been made. Treating can include, for example, reducing, delaying or alleviating the severity of one or more symptoms of the disease or condition, or it can include reducing the frequency with which symptoms of a disease, defect, disorder, or adverse condition, and the like, are experienced by a patient. Treating can be used herein to refer to a method that results in some level of treatment or amelioration of the disease or condition, and can contemplate a range of results directed to that end, including but not restricted to prevention of the condition entirely.

In certain embodiments, the term “prevent” or “preventing” as related to a disease or disorder may refer to a compound that, in a statistical sample, reduces the occurrence of the disorder or condition in the treated sample relative to an untreated control sample, or delays the onset or reduces the severity of one or more symptoms of the disorder or condition relative to the untreated control sample.

A “therapeutic effect,” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

### Compounds

In some aspects. the present disclosure provides a compound represented by the structure of Formula (I):

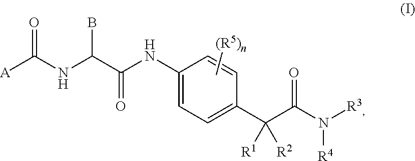

or a pharmaceutically acceptable salt thereof, wherein:
A is selected from a 5- to 6-membered heterocycle optionally substituted with one more substituents independently selected from (i), (ii), and (iii):
   (i) halogen, —OR¹¹, —SR¹¹, —N(R¹¹)₂, —C(O)R¹¹, —C(O)OR¹¹, —OC(O)R¹¹, —OC(O)N(R¹¹)₂, —C(O)N(R¹¹)₂, —N(R¹¹)C(O)R¹¹, —N(R¹¹)C(O)OR¹¹,—N(R¹¹)C(O)N(R¹¹)₂, —N(R¹¹)S(O)₂(R¹¹), —S(O)R¹¹, —S(O)₂R¹¹, —S(O)₂N(R¹¹)₂, —NO₂, and —CN;
   (ii) C₁₋₁₀ alkyl optionally substituted with one or more substituents independently selected from:
      halogen, —OR¹¹, —SR¹¹, —N(R¹¹)₂, —C(O)R¹¹, —C(O)OR¹¹, —OC(O)R¹¹, —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹¹)₂, —N(R¹¹)S(O)₂(R¹¹), —S(O)R¹¹, —S(O)₂R¹¹, —S(O)₂N(R¹¹)₂, —NO₂, ═O, —CN; and
      C₃₋₁₀ carbocycle and 3- to 10-membered heterocycle each of which is optionally substituted with one or more substituents selected from: halogen, —OR¹¹, —SR¹¹, —N(R¹¹)₂, —C(O)R¹¹, —C(O)OR¹¹, —OC(O)R¹¹, —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹¹)₂, —N(R¹¹)S(O)₂(R¹¹), —S(O)R¹¹, —S(O)₂R¹¹, —S(O)₂N(R¹¹)₂, —NO₂, ═O, and —CN; and
   (iii) 3- to 10-membered heterocycle optionally substituted with one or more substituents independently selected from:
      halogen, —OR¹¹, —SR¹¹, —N(R¹¹)₂, —C(O)R¹¹, —C(O)OR¹¹, —OC(O)R¹¹, —OC(O)N(R¹¹)z —C(O)N(R¹¹)₂, —N(R¹¹)C(O)R¹¹, —N(R¹¹)C(O)OR¹¹, —N(R¹¹)C(O)N(R¹¹)₂, —N(R¹¹)S(O)₂(R¹¹), —S(O)R¹¹, —S(O)₂R¹¹, —S(O)₂N(R¹¹)₂, —NO₂, ═O, —CN; and
      C₁₋₆ alkyl optionally substituted with one or more substituents independently selected from halogen, —OR¹¹, —SR¹¹, —N(R¹¹)₂, —C(O)R¹¹, —C(O)OR¹¹, —OC(O)R¹¹, —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹¹)₂, —N(R¹¹)S(O)₂(R¹¹), —S(O)R¹¹, —S(O)₂R¹¹, —S(O)₂N(R¹¹)₂, —NO₂—═O, and —CN;
B is selected from —CH(R^{A})(R¹¹) and C₃₋₁₀ carbocycle, wherein the C₃₋₁₀ carbocycle is optionally substituted with one or more substituents independently selected from:
   halogen, —OR², —SR¹², —N(R²)₂, —C(O)R², —C(O)OR¹¹, —OC(O)R¹¹, —OC(O)N(R¹²)₂, —C(O)N(R¹¹)₂, —N(R¹²)C(O)R¹²—N(R¹²)C(O)OR¹², —N(R¹²)C(O)N(R¹¹)₂, —N(R¹²)S(O)₂(R¹²), —S(O)R¹², —S(O)₂R², —S(O)₂N(R¹¹)₂, —NO₂, ═O, and —CN; and
   C₁₋₆ alkyl optionally substituted with one or more substituents independently selected from halogen, —OR¹², —SR¹², —N(R¹²)₂, —C(O)R¹², —C(O)OR¹², —OC(O)R¹², —OC(O)N(R¹²)₂, —C(O)N(R²)₂, —N(R^{11B})C(O)R¹²—N(R¹²)C(O)OR¹², —N(R¹²)C(O)N(R¹²)₂, —N(R¹²)S(O)₂(R¹²), —S(O)R², —S(O)₂R¹², —S(O)₂N(R²)₂, —NO₂, ═O, and —CN; R^{A} and R^{B} are each independently selected from:
      halogen, —OR¹¹, —SR¹³, —N(R¹¹)₂, —C(O)R¹³, —C(O)OR¹¹, —OC(O)R¹¹, —OC(O)N(R¹³)₂—C(O)N(R¹³)₂, —N(R^{1′})C(O)R¹¹, —N(R¹¹)C(O)OR¹³, —N(R¹¹)C(O)N(R¹³)₂, —N(R¹³)S(O)₂(R¹³), —S(O)R¹¹)₃, —S(O)₂R¹¹, —S(O)₂N(R¹¹)₃)₂, —NO₂, and —CN; C₁₋₆ alkyl optionally substituted with one or more substituents independently selected from halogen, —OR¹², —SR¹³, —N(R¹¹)₂, —C(O)R¹¹, —C(O)OR¹¹, —OC(O)R¹¹, —OC(O)N(R¹⁴)₂, —C(O)N(R¹³)₂, —N(R¹³)C(O)R¹³, —N(R¹³)C(O)OR¹³, —N(R¹³)C(O)N(R¹³)₂, —N(R^{1D})₂S(O)₂(R¹¹), —S(O)R¹³, —S(O)₂R¹³, —S(O); N(R³);, —NO₂, and —CN; and C₃₋₁₀ carbocycle optionally substituted with one or more substituents independently selected from halogen, —OR¹², —SR¹, —N(R¹¹)₂, —C(O)R¹³, —C(O)OR¹¹, —OC(O)R¹¹, —OC(O)N(R¹¹)₂, —C(O)N(R¹³)₂, —N(R¹¹)C(O)R¹³, —N(R¹³)C(O)OR¹³, —N(R^{1′})C(O)N(R^{1′})₂, —N(R¹¹)S(O)₂(R¹¹), —S(O)R¹¹, —S(O)₂R¹¹, —S(O)₂N(R¹¹)₂, —NO₂, and —CN; R¹ and R² are each independently selected from (iv), (v), (vi) and (vii):
   (iv) hydrogen;
   (v) halogen, —OR¹⁴, —SR¹⁴, —N(R¹⁴)₂, —C(O)R¹⁴, —C(O)OR¹⁴, —OC(O)R¹⁴, —OC(O)N(R^{11B}), —C(O)N(R¹⁴)₂, —N(R¹⁴)C(O)R¹⁴, —N(R¹⁴)C(O)OR¹⁴, —N(R¹⁴)C(O)N(R¹⁴)₂, —N(R¹⁴)S(Oh(R¹⁴), —S(O)R¹⁴, —S(O)₂R¹⁴, —S(O)₂N(R¹⁴)₂, —NO₂, and —CN;
   (vi) C₁₋₆ alkyl optionally substituted with one or more substituents independently selected from:
      halogen, —OR¹⁴, —SR¹⁴, —N(R¹⁴)₂, —C(O)R¹⁴, —C(O)OR¹⁴, —OC(O)R¹⁴, —OC(O)N(R¹⁴)₂, —C(O)N(R¹⁴)₂, —N(R¹⁴)C(O)R¹⁴, —N(R¹⁴)C(O)OR¹⁴, —N(R¹⁴)C(O)N(R¹⁴)₂, —N(R¹⁴)S(O)₂(R¹⁴), —S(O)R¹⁴, —S(O)₂R¹⁴, —S(O)₂N(R¹⁴), —NO₂, and —CN; and
      3- to 10-membered heterocycle optionally substituted with one or more substituents independently selected from halogen, —OR^{14B}, —SR¹⁴, —N(R^{14F})₂, —C(O)R¹⁴, —C(O)OR¹⁴, —OC(O)R^{14B}, —OC(O)N(R¹¹)₂, —C(O)N(RB), —N(R^{14B})C(O)R^{14B}, —N(R^{14D})C(O)OR^{14B}, —N(R^{14B})C(O)N(R^{14B})₂, —N(R^{14B})S(O)₂(R^{14B}), —S(O)R¹⁴, —S(O)₂R¹⁴, —S(O)₂N(R^{11B})₂, —NO₂, and —CN; and
   (vii) 3- to 10-membered heterocycle optionally substituted with one or more substituents independently selected from:
      halogen, —OR^{14B}, —SR^{14B}, —N(R^{14B})₂, —C(O)R^{14B}, —C(O)OR^{14B}, —OC(O)R^{14B}, —OC(O)N(R^{14B})₂, —C(O)N(R^{14B})₂, —N(R^{14B})C(O)R¹⁴, —N(R^{14B})C(O)OR^{14B}—N(R^{14B})C(O)N(R^{14B})₂—N(R^{14B})S(O)₂(R^{14B}), —S(O)R^{14B}, —S(O)₂R⁴¹, —S(O)₂N(R^{14B})₂, —NO₂, and —CN, R³ and R⁴ are each independently selected from (a), (b), (c), and (d):
   (a) hydrogen;
   (b) C, alkyl optionally substituted with one or more substituents independently selected from:
      halogen, —OR¹¹, —SR¹¹, —N(R¹¹)₂, —C(O)R¹¹, —C(O)NH₂, —C(O)—O-C₁₋₆alkyl, —OC(O)R¹¹, —OC(O)N(R¹¹)₂, —N(R^{1s})C(O)R¹¹, —N(R¹⁴)C(O)OR¹⁵, —N(R¹⁵)C(O)N(R¹⁵), —N(R¹⁴)S(O)₂(R¹⁵), —S(O)R¹¹, —S(O)₂R¹², —S(O)₂N(R¹⁵)₂, —NO₂, —CN; C₃₋₁₀ carbocycle and 3- to 10-membered heterocycle any of which is optionally substituted with one or more substituents independently selected from halogen, C₁₋₆ haloalkyl, C₁₋₆ alkoxy, —OR¹¹, —SR¹¹, —N(R¹⁴)₂, —C(O)R¹⁵, —C(O)OR¹⁵, —OC(O)R¹¹, —OC(O)N(R¹¹)₂, —C(O)N(R¹⁵)₂, —N(R¹¹)C(O)R¹¹, —N(R¹¹)C(O)OR¹¹, —N(R¹⁴)C(O)N(R¹¹)₂, —N(R¹¹)S(O)₂(R¹⁵), —S(O)R¹⁵, —S(O)₂R¹⁵, —S(O)₂N(R¹⁵), —NO₂, and —CN;
   (c) C₂₋₆ alkyl optionally substituted with one or more substituents independently selected from:
      halogen, —OR¹¹, —SR¹¹, —N(R¹¹)₂, —C(O)R¹⁴, —C(O)—O—C₁₋₆-alkyl, —OC(O)R¹¹, —OC(O)N(R¹¹)₂, —C(O)N(Ru)₂, —N(R¹⁶)C(O)R¹¹, —N(R¹¹)C(O)OR¹¹, —N(R¹¹)C(O)N(R¹¹)₂, —N(R¹¹)₅)S(O)₂(R¹¹), —S(O)R¹¹, —S(O)₂R¹⁵, —S(O)₂N(R¹⁵)₂, —NO₂, and —CN;
      C₃₋₁₀ carbocycle and 3- to 10-membered heterocycle any of which is optionally substituted with one or more substituents independently selected from halogen, C,6 haloalkyl, C₁₋₄ alkoxy, —OR¹⁵, —SR¹¹, —N(R¹¹)₂, —C(O)R¹¹, —C(O)OR¹¹, —OC(O)R¹¹, —OC(O)N(R¹¹)z, —C(O)N(R¹¹)₂, —N(R¹¹)C(O)R¹¹, —N(R¹¹)C(O)OR¹²,s —N(R^{11B})C(O)N(R¹¹)₂, —N(R⁵)S(O)₂(R¹¹), —S(O)R¹¹, —S(O)₂R¹⁵, —S(O)₂N(R¹¹)₂, —NO₂, and —CN; and
   (d) C₃₋₁₀ carbocycle and 3- to 10-membered heterocycle any of which is optionally substituted with one or more substituents independently selected from:
      halogen, —OR¹¹, —SR¹¹, —N(R¹¹)₂, —C(O)R¹¹, —C(O)OR¹¹, —OC(O)R¹¹, —OC(O)N(R¹¹)₂, —C(O)N(R¹¹)z, —N(R¹¹)C(O)R¹⁴, —N(R¹⁴)C(O)OR¹², —N(R¹¹)₃)C(O)N(R¹¹)₂, —N(R¹¹)S(O)₂(R¹¹), —S(O)R¹⁵, —S(O)₂R¹⁵, —S(O)₂N(R¹¹)₂, —NO₂, and —CN; and
      C₁₋₆ alkyl optionally substituted with one or more substituents independently selected from halogen, —OR¹⁵, —SR¹², —N(R¹¹)₂, —C(O)R¹¹)₅, —C(O)OR¹³, —OC(O)R¹¹, —OC(O)N(R¹¹)₂—C(O)N(R¹¹)₂, —N(R¹¹)C(O)R¹¹, —N(R¹¹)C(O)OR¹¹, —N(R¹⁵)C(O)N(R¹⁴)₂, —N(R¹⁴)S(O)₂(R¹¹), —S(O)R¹¹)₅, —S(O)₂R¹¹, —S(O)₂N(R¹¹)₅)₂, —NO₂, and —CN; or
R³ and R⁴ can come together to form a 4- to 12-membered heterocycle optionally substituted with one or more substituents independently selected from:
   halogen, —OR¹⁶, —SR¹⁶, —N(R¹⁶)₂, —C(O)R⁶, —C(O)OR¹⁶, —OC(O)R¹⁶, —OC(O)N(R¹⁶)₂, —C(O)N(R6)₂, —N(R¹⁶)C(O)R⁶, —N(R¹⁶)C(O)OR¹⁶, —N(R¹⁶)C(O)N(R6)₂, —N(R¹⁶)S(O)₂(R¹⁶), —S(O)R¹⁶, —S(O)₂R¹⁶, —S(O)₂N(R¹⁶)₂, —NO₂, ═O, and —CN; and
   C₁₋₆ alkyl optionally substituted with one or more substituents independently selected from halogen, —OR¹⁶, —SR¹⁶, —N(R¹⁶)₂, —C(O)R¹⁶, —C(O)OR¹⁶, —OC(O)R¹⁶, —OC(O)N(R¹⁶)₂, —C(O)N(R¹⁶)₂, —N(R¹⁶)C(O)R¹⁶, —N(R¹⁶)C(O)OR,⁶—N(R¹⁶)C(O)N(R¹⁶)₂, —N(R¹⁶)S(O)₂(R¹⁶), —S(O)R¹⁶, —S(O)₂R¹⁶, —S(O)₂N(R¹⁶)₂, —NO₂, ═O, and —CN;
R⁵ is selected from:
   halogen, —OR¹⁷, —SR¹⁷, —N(R¹⁷)₂, —C(O)R¹¹, —C(O)OR¹¹, —OC(O)R¹⁷, —OC(O)N(R¹¹)₂, —C(O)N(R¹¹)₂, —N(R¹¹)C(O)R¹⁴, —N(R¹¹)C(O)OR¹¹, —N(R¹¹)₇)C(O)N(R¹¹)₂, —N(R¹¹)S(O)₂(R¹¹). —S(O)R¹⁷, —S(O)₂R¹⁷, —S(O)₂N(R¹⁷)₂, —NO₂, and —CN;
      C₁₋₆ alkyl and C₂₋₆ alkenyl each of which is optionally substituted with one or more substituents independently selected from halogen, —OR¹¹, —SR¹¹, —N(R¹¹)₂, —C(O)R¹⁷, —C(O)OR¹⁷, —OC(O)R¹⁷, —OC(O)N(R¹¹)₂, —C(O)N(R¹¹)₂, —N(R¹¹)C(O)R¹⁷, —N(R¹⁷)C(O)OR¹¹, —N(R¹⁷)C(O)N(R¹¹)₂, —N(R¹⁷)S(O)₂(R¹⁷), —S(O)R¹⁷, —S(O)₂R¹⁷, —S(O)₂N(R¹⁷)₂, —NO₂, ═O, and —CN; and
      C₃₋₁₀ carbocycle optionally substituted with one or more substituents independently selected from halogen, —OR¹⁷B, —SR^{17B}, —N(R¹¹)₇)₂, —C(O)R¹⁷, —C(O)OR¹¹)₇, —OC(O)R¹¹)_{7B}, —OC(O)N(R¹⁴)₂, —C(O)N(R^{17B})₂, —N(R^{17B})C(O)R 7, —N(R^{17B})C(O)OR¹⁷, —N(R^{17B})C(O)N(R¹⁴)₂, —N(R^{14B})S(O)₂(R^{17B}), —S(O)R^{14B}, —S(O)₂R¹¹, —S(O)₂N(R^{17B})₂, —NO₂, and —CN;
         R¹¹, R², R¹³, R¹⁴, R^{14B}. R⁵, R¹⁶, R¹⁷, and R^{1I} are independently selected at each occurrence from (1), (II) and (111):
(I) hydrogen;
(II) C₁₋₆ alkyl optionally substituted with one or more substituents independently selected from: halogen, —OH, —O—C₁₋₆ alkyl, —O—C₁₋₆ haloalkyl —NH₂, —NO₂, ═O, —CN, C₃₋₁₀ carbocycle and 3 to 10-membered heterocycle, wherein the C₃₋₁₀ carbocycle and 3- to 10-membered heterocycle are optionally substituted with one or more substituents selected from: halogen, —OH, C₁-C₆ haloalkyl, —O—C₁-C₆ alkyl, —O—C₁-C₆haloalkyl —NH₂, —NO₂, ═O, and —CN; and (III) C₃₋₁₀ carbocycle and 3- to 10-membered heterocycle, each of which is optionally substituted with one or more substituents independently selected from: halogen, —OH, C₁-C₆ haloalkyl, —O—C₁-C₆ alkyl, —O—C₁-C₆haloalkyl —NH₂, —NO₂, ═O, and —CN; and C₁-6 alkyl optionally substituted with one or more substituents independently selected from halogen, —OH, —O—C₁-C₆ alkyl, —O—C₁-C₆haloalkyl —NH₂, —NO₂, ═O, and —CN; and n is selected from 0, 1, 2, 3, and 4, wherein n is selected from 1, 2, 3, and 4; or at least one of R¹ or R² is hydrogen.

In some embodiments, for the compound or salt of Formula (I), R⁵ is selected from C₃—.o carbocycle optionally substituted with one or more substituents independently selected from halogen, —OR^{4B}, —SR^{11B}, —N(R¹⁴′)₂, —C(O)R¹¹)_{B}, —C(O)OR¹¹¹, —OC(O)R^{4m}, —OC(O)N(R¹¹)₂.—C(O)N(R^{17B})₂, —N(R^{M})C(O)Rⁿ, —N(R¹⁷)C(O)OR^{B}, —N(R^{M})C(O)N(R^{17B})₂, —N(R^{17B})S(O)₂(R¹¹)_{B}), —S(O)R¹¹)_{B}, —S(O)₂R¹¹)_{B}, —S(O)₂N(R¹¹)_{B})₂, —NO₂, and —CN. In some embodiments, R⁵ is selected from C₃₋₆ carbocycle optionally substituted with one or more substituents independently selected from halogen, —OR^{11B}, —N(R^{1M})₂, —C(O)R¹¹)_{B}, —C(O)OR¹¹m, —NO₂, and —CN.

In some embodiments, R⁵ is an optionally substituted saturated C₃₋₆ carbocycle. In some embodiments. R⁵ is an optionally substituted C₆ aryl. In some embodiments, R⁵ is selected from cyclopropyl, cyclobutyl, cyclopentyl, cyclohexyl, and phenyl, any one of which is optionally substituted. In some embodiments, R⁵ is optionally substituted cyclopropyl. In some embodiments, R⁵ is represented by:

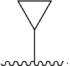

In some embodiments, for the compound or salt of Formula (I), one of R¹ or R² is selected from: C₁₋₆ alkyl optionally substituted with one or more substituents independently selected from 3 to 10-membered heterocycle optionally substituted with one or more substituents independently selected from halogen, —OR¹⁶, —SR^{14B}, —N(R^{14B})₂, —C(O)R¹⁴¹, —C(O)OR^{1B}, —OC(O)R¹⁴, —OC(O)N(R¹⁴)₂, —C(O)N(R^{14B})₂, —N(R^{14B})C(O)R^{14B}, —N(R^{14B})C(O)OR^{14B}, —N(R^{1B})C(O)N(R¹⁴ⁿ)₂, —N(R¹⁴)S(O)₂(R^{14B}), —S(O)R^{14B}, —S(O)₂R,^{4B}—S(O)₂N(R¹⁴)₂, —NO₂, and —CN; and 3- to 10-membered heterocycle optionally substituted with one or more substituents independently selected from: halogen, —OR¹⁴B, —SR^{14B}, —N(R¹⁴ⁿ)₂, —C(O)R^{14B}, —C(O)OR^{14B}, —OC(O)R¹⁴1, —OC(O)N(R^{14B}E-C(O)N(R^{14B})₂, —N(R¹⁴ⁿ)C(O)R^{14B}, —N(R^{14B})C(O)OR^{14B}, —N(R^{14B})C(O)N(R^{14B})₂—N(R¹⁴)S(O)₂(R^{14B}), —S(O)R^{14B}, —S(O)₂R^{14B}, —S(O)₂N(R¹¹)₂, —NO₂, and —CN.

In some embodiments, for the compound or salt of Formula (I), one of R¹ or R² is selected from: methyl, ethyl. propyl, and isopropyl, each of which is optionally substituted with one or more substituents independently selected from 3- to 10-membered heterocycle optionally substituted with one or more substituents independently selected from halogen, —OR¹⁴¹, —SR¹⁴, —N(R¹⁴)₂, —C(O)R^{14B}, —C(O)OR^{14B}, —OC(O)R^{14B}, —OC(O)N(R^{14B})₂, —C(O)N(R^{1aB})₂, —N(R^{14D})C(O)R^{14B}—N(R¹⁴)C(O)OR^{14B}, —N(R¹⁴ⁿ)C(O)N(RIB)₂, —N(R¹⁴)S(O)₂(R^{4B}), —S(O)R^{14B}, —S(O)₂R4, —S(O)₂N(R^{14B})₂, —NO₂, and —CN; and 3- to 10-membered heterocycle optionally substituted with one or more substituents independently selected from: halogen, —OR^{14B}, —SR^{14B}, —N(R^{14B})₂, —C(O)R^{14B}, —C(O)OR^{14B}, —OC(O)R^{14B}, —OC(O)N(R^{17B})₂, —C(O)N(R^{14B})₂,—N(R^{14B})C(O)RIB. —N(R^{14B})C(O)OR^{14B}, —N(R^{14B})C(O)N(R^{14B})₂, —N(R^{14B})S(O)₂(RB), —S(O)R¹⁴R —S(O)₂R4B, —S(O)₂N(R¹⁴)₂, —NO₂, and —CN.

In some embodiments, for the compound or salt of Formula (I), R¹ is hydrogen; and R² is selected from: C₁₆ alkyl optionally substituted with one or more substituents independently selected from 3- to 10-membered heterocycle optionally substituted with one or more substituents independently selected from halogen, —OR^{14B}, —SR^{14B}, —N(R^{14B})₂, —C(O)R^{14B}, —C(O)OR^{14B}, —OC(O)R^{14B}, —OC(O)N(R^{14B})₂, —C(O)N(RB)₂, —N(R¹⁴ⁿ)C(O)R^{14B}, —N(R^{14B})C(O)OR¹⁴n, —N(R¹⁴)C(O)N(R^{14B})₂, —N(R^{14B})S(O)₂(R^{14B}) —S(O)R^{14B}—S(O)₂R,⁴B —S(O)₂N(RB)₂, —NO₂, and —CN; and 3- to 10-membered heterocycle optionally substituted with one or more substituents independently selected from: halogen, —OR^{14B}, —SR^{14B}, —N(R^{14B})₂, —C(O)R¹⁴ⁿ, —C(O)OR^{14B}, —OC(O)R^{14B} —OC(O)N(R^{14B}), —C(O)N(R¹⁴)₂, —N(R¹⁴)C(O)R^{14B}—N(R^{14B})C(O)OR^{14B}, —N(R¹⁴n)C(O)N(R¹⁴1)₂, —N(R¹⁴1)S(O)₂(R^{14B}), —S(O)R¹⁴1, —S(O)₂R^{4B}, —S(O)₂N(R^{14B})₂, —NO₂, and —CN.

In some embodiments, for the compound or salt of Formula (I), one of R¹ or R² is selected from: C₁-6 alkyl optionally substituted with one or more substituents independently selected from 5 to 6-membered heterocycle optionally substituted with one or more substituents independently selected from halogen, —OR^{14B}, —N(R^{14B})₂, —C(O)R¹⁴¹, —C(O)OR^{14B}, —NO₂, and —CN. In some embodiments, one of R¹ or R² is Ci₆ alkyl substituted with a 5- to 6-membered saturated heterocycle wherein the 5- to 6-membered saturated heterocycle selected from pyrrolidine, pyrroline, pyrazoline, imidazoline, tetrahydrofuran, dioxolane, tetrahydrothiophene, piperidine, piperazine, tetrahydropyran, dioxane, thiane, dithiane, morpholine, and thiomorpholine, any one of

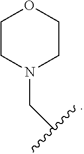 [SMILES: *CN1CCOCC1]

which is optionally substituted. In some embodiments, one of R¹ or R² is represented by.

In some embodiments, for the compound or salt of Formula (I), R¹ is hydrogen; and R² is selected from: C₁₋₆ alkyl optionally substituted with one or more substituents independently selected from 5- to 6-membered heterocycle optionally substituted with one or more substituents independently selected from halogen, —OR^{11B}, —N(R^{14B})₂, —C(O)R^{14B}, —C(O)OR^{14B}, —NO₂, and —CN. In some embodiments, R¹ is hydrogen; and R² is C₁₋₆ alkyl substituted with a 5- to 6-membered saturated heterocycle wherein the 5- to 6-membered saturated heterocycle selected from pyrrolidine, pyrroline, pyrazoline, imidazoline, tetrahydrofuran, dioxolane, tetrahydrothiophene, piperidine, piperazine, tetrahydropyran, dioxane, thiane, dithiane, morpholine, and thiomorpholine, any one of which is optionally substituted. In some embodiments, R¹ is hydrogen; and R² is represented by

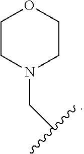 [SMILES: *CN1CCOCC1]

In some embodiments, for the compound or salt of Formula (I), one of R¹ or R² is a 5- to 6-membered heterocycle optionally substituted with one or more substituents independently selected from: halogen, —OR^{14B}, —N(R^{14B})₂, —C(O)R^{14H}, —C(O)OR^{14B}, —NO₂, and —CN. In some embodiments, one of R¹ or R² is 5- to 6-membered heterocycle selected from pyrrolidine, pyrroline, pyrazoline, imidazoline, tetrahydrofuran, dioxolane, tetrahydrothiophene, piperidine, piperazine, tetrahydropyran, dioxane, thiane, dithiane, morpholine, and thiomorpholine, any one of which is

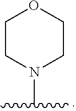 [SMILES: *N1CCOCC1]

optionally substituted. In some embodiments, one of R¹ or R² is represented by

In some embodiments, for the compound or salt of Formula (I), R¹ is hydrogen; and R² is a 5- to 6-membered heterocycle optionally substituted with one or more substituents independently selected from: halogen, —OR¹⁴, —N(R^{14B})₂, —C(O)R^{14H}, —C(O)OR^{14B}, —NO₂, and —CN. In some embodiments, R¹ is hydrogen; and R² is 5- to 6-membered heterocycle selected from pyrrolidine, pyrroline, pyrazoline, imidazoline, tetrahydrofuran, dioxolane, tetrahydrothiophene, piperidine, piperazine, tetrahydropyran, dioxane, thiane, dithiane, morpholine, and thiomorpholine, any one of which is optionally substituted. In some embodiments, R¹ is hydrogen; and R² is represented by

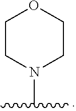 [SMILES: *N1CCOCC1]

In some embodiments, for compound or salt of Formula (I). one of R³ and R⁴ is selected from C₁ alkyl optionally substituted with one or more substituents independently selected from:
halogen, —OR¹¹, —SR¹¹, —N(R¹¹)₂, —C(O)R¹⁵, —C(O)NH₂, —C(O)—O—C₁.(-alkyl, —OC(O)R¹¹, —OC(O)N(R¹¹)₂, —N(R¹¹)C(O)R¹¹, —N(R¹¹)C(O)OR¹¹, —N(R¹¹)C(O)N(R¹¹)₂, —N(R¹¹)S(O)₂_(R¹¹), —S(O)R¹¹, —S(O)₂R¹¹, —S(O)₂N(R¹¹)₂, —NO₂, —CN;
C₃₋₁₀ carbocycle and 3- to 10-membered heterocycle any of which is optionally substituted with one or more substituents independently selected from halogen, C₁₋₆ haloalkyl, C₁₋₆ alkoxy, —OR¹¹, —SR¹¹, —N(R¹¹)₂, —C(O)R¹¹, —C(O)OR¹¹, —OC(O)R¹¹, —OC(O)N(R¹¹)₂, —C(O)N(R¹¹)₂, —N(R¹¹)C(O)R¹¹, —N(R¹¹)C(O)OR¹¹, —N(R⁵)C(O)N(R¹¹)₅)₂, —N(R¹¹)S(O)₂(R¹¹), —S(O)R¹¹, —S(O)₂R¹², —S(O)₂N(R¹¹)₂, —NO₂, and —CN.

In some embodiments, one of R³ and R⁴ is selected from C₁ alkyl optionally substituted with one or more substituents independently selected from halogen, —OR⁵, —N(R¹¹)₂, —C(O)R¹¹, —C(O)NH₂, —NO₂, and —CN. In some embodiments, one of R³ and R⁴ is selected from Ct alkyl optionally substituted with —C(O)NH₂. In some embodiments, one of R³ and R⁴ is represented by

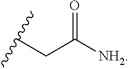 [SMILES: *CC(N)=O]

In some aspects, the present disclosure provides a compound represented by the structure of Formula (I-a):
or a pharmaceutically acceptable salt thereof, wherein:

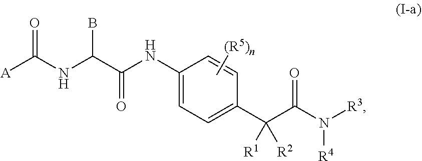

A is selected from a 5- to 6-membered heterocycle optionally substituted with one more substituents independently selected from (i), (ii), and (iii):
(i) halogen, —OR¹¹, —SR¹¹, —N(R¹¹)₂, —C(O)R¹¹, C(O)OR¹¹, —OC(O)R¹¹, —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹¹)₂, —N(R¹¹)S(O)₂(R¹¹),—S(O)R¹¹, —S(O)₂R¹¹,—S(O)₂N(R¹¹)₂, —NO₂, and —CN;
(ii) C₁₋₁₀ alkyl optionally substituted with one or more substituents independently selected from;
   halogen, —OR¹¹, —SR¹¹, —N(R¹¹)₂, —C(O)R¹¹, C(O)OR¹¹, —OC(O)R¹¹, —OC(O)N(R¹¹)—C(O)N(R¹¹)₂, —N(R¹¹)C(O)R¹¹, —N(R¹¹)C(O)OR¹¹, —N(R¹¹)C(O)N(R¹¹)₂, —N(R¹¹)S(O)₂(R¹¹), —S(O)R¹¹, —S(O)₂R, —S(O)₂N(R¹¹)₂, —NO₂, ═O, —CN; and
   C₃₋₁₀ carbocycle and 3- to 10-membered heterocycle each of which is optionally substituted with one or more substituents selected from: halogen, —OR¹¹, —SR¹¹, —N(R¹¹)₂, —C(O)R¹¹, —C(O)OR¹¹, —OC(O)R¹¹, —OC(O)N(R¹¹)₂, —C(O)N(R¹¹)₂,—N(R¹¹)C(O)R¹¹, —N(R¹¹)C(O)OR¹¹, —N(R¹¹)C(O)N(R¹¹)₂, —N(R¹¹)S(O)₂(R¹¹), —S(O)R¹¹, —S(O)₂R¹¹, —S(O)₂N(R¹¹)₂, —NO₂, ═O, and —CN; and
(iii) 3- to 10 membered heterocycle optionally substituted with one or more substituents independently selected from:
halogen, —OR¹¹, —SR¹¹, —N(R¹¹)₂, —C(O)R¹¹, —C(O)OR¹¹, —OC(O)R¹¹, —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹¹)₂, —N(R¹¹)S(O)₂(R¹¹), —S(O)R¹¹, —S(O)₂R¹¹, —S(O)₂N(R¹², —NO₂, ═O, —CN; and
   C₁₋₆ alkyl optionally substituted with one or more substituents independently selected from halogen, —OR¹¹, —SR¹¹, —N(R¹¹)₂, —C(O)R¹¹, —C(O)OR¹¹, —OC(O)R¹¹, —OC(O)N(R¹¹). —C(O)N(R¹², —N(R¹¹)C(O)R¹¹, —N(R¹¹)C(O)OR¹¹, —N(R¹¹)C(O)N(R¹¹)₂, —N(R¹¹)S(O)₂(R¹¹), —S(O)R¹¹, —S(O)₂R¹¹, —S(O)₂N(R¹¹)₂, —NO₂, ═O, and —CN;
B is selected from —CH(R^{A})(R^{B}) and C,o carbocycle, wherein C₃₋₁₀ carbocycle is optionally substituted with one or more substituents independently selected from:
   halogen, —OR¹², —SR¹², —N(R¹²)₂, —C(O)R¹², —C(O)OR¹², —OC(O)R¹², —OC(O)N(R², —C(O)N(R)₂, —N(R^{1z})C(O)R^{1z}, —N(R^{1z})C(O)OR¹², —N(R²)C(O)N(R²)₂, —N(R^{1z})S(O)₂(R¹¹), —S(O)R¹⁴, —S(O)₂R², —S(O)₂N(R¹²)₂, —NO₂, ═O, and —CN; and C₁₋₆ alkyl optionally substituted with one or more substituents independently selected from halogen, —OR¹², —SR¹², —N(R¹²)₂, —C(O)R¹², —C(O)OR¹², —OC(O)R¹², —OC(O)N(R¹²)₂, —C(O)N(R¹²)₂, —N(R¹⁴)C(O)R¹², —N(R¹²)C(O)OR¹², —N(R¹²)C(O)N(R¹²)₂, —N(R¹²)S(O)₂(R²), —S(O)R², —S(O)₂R², —S(O)₂N(R¹²)₂, —NO₂, =O, and —CN;
R^{A} and R^{B} are each independently selected from:
   halogen, —OR¹⁴, —SR¹¹, —N(R¹³)₂, —C(O)R¹¹, —C(O)OR¹², —OC(O)R¹¹, —OC(O)N(R¹³)₂.—C(O)N(RD)₂, —N(R¹¹)C(O)R3, —N(R¹¹)C(O)OR³, —N(R¹³)C(O)N(R¹¹)₂, —N(R3)S(O)₂(R¹¹), —S(O)R¹¹, —S(O)₂R³, —S(O)₂N(R¹¹)₂, —NO₂, and —CN;
      C₁₋₁₀ alkyl optionally substituted with one or more substituents independently selected from halogen, —OR¹², —SR¹³, —N(R¹¹)₂, —C(O)R³, —C(O)OR¹¹, —OC(O)R¹¹, —OC(O)N(R¹¹)₂, —C(O)N(R¹³)₂, —N(R¹¹)C(O)R¹¹, —N(R¹¹)C(O)OR^{1′}, —N(R¹¹)C(O)N(R¹¹)₂, —N(R¹¹)₂S(O)₂(R¹¹), —S(O)R¹¹, —S(O)₂R¹¹, —S(O)₂N(R¹¹), —NO₂, and —CN; and
      C₃₋₁₀ carbocycle optionally substituted with one or more substituents independently selected from halogen, —OR³, —SR¹³, —N(R¹¹)₂, —C(O)R¹¹, —C(O)OR¹¹, —OC(O)R¹¹, —OC(O)N(R¹¹)₂, —C(O)N(R¹¹)₂, —N(R¹¹)C(O)R^{1′}, —N(R¹¹)C(O)OR¹¹, —N(R)C(O)N(R)₂, —N(R¹³)S(O)₂(R¹³), —S(O)R¹¹, —S(O)₂R¹¹, —S(O)₂N(R¹¹)₂, —NO₂, and —CN;
R¹ and R² are each independently selected from:
   hydrogen;
   halogen, —OR¹¹, —SR¹, —N(R¹⁴)₂, —C(O)R¹⁴, —C(O)OR¹⁴, —OC(O)R¹⁴, —OC(O)N(R¹⁴)₂, —C(O)N(R¹⁴)₂, —N(R¹⁴)C(O)R¹⁴, —N(R¹⁴)C(O)OR¹⁴, —N(R¹¹)C(O)N(R¹⁴)₂, —N(R¹⁴)S(O)₂(R¹⁴), —S(O)R¹⁴, —S(O)₂R¹⁴, —S(O)₂N(R¹¹)₂, —NO₂, and —CN; and
   C₁₋₆ alkyl optionally substituted with one or more substituents independently selected from halogen, —OR¹⁴, —SR¹⁴, —N(R¹⁴)₂, —C(O)R¹⁴, —C(O)OR¹⁴, —OC(O)R¹⁴, —OC(O)N(R¹⁴)₂, —C(O)N(R¹⁴)₂, —N(R¹⁴)C(O)R¹⁴, —N(R¹⁴)C(O)OR¹¹, —N(R¹⁴)C(O)N(R¹⁴)₂, —
N(R¹⁴)S(O)₂(R¹⁴), —S(O)R¹⁴, —S(O)₂R¹⁴, —S(O)₂N(R¹⁴)₂, —NO₂, and —CN;
R³ and R⁴ are each independently selected from (a), (b), (c), and (d):
   (a) hydrogen;
   (b) C, alkyl optionally substituted with one or more substituents independently selected from:
      halogen, —OR¹¹, —SR¹², —N(R¹¹)₂, —C(O)R¹¹, —C(O)—O—C₁₋₆.alkyl, —OC(O)R^{11s}, —OC(O)N(R¹¹)₂, —N(R¹¹)C(O)R¹⁴, —N(R¹¹)₅)C(O)OR¹¹, —N(R¹¹)C(O)N(R¹¹)₂, —N(R¹¹)S(O)₂(R¹¹), —S(O)R¹¹, —S(O)₂R¹¹, —S(O)₂N(R¹¹)₂, —NO₂, —CN;
      C₃₋₁₀ carbocycle and 3- to 10-membered heterocycle any of which is optionally substituted with one or more substituents independently selected from halogen, C,- haloalkyl, C,6 alkoxy, —OR¹¹, —SR¹¹, —N(R¹¹)₂, —C(O)R¹¹, —C(O)OR¹¹, —OC(O)R¹¹, —OC(O)N(R¹¹)₂, —C(O)N(R¹¹)₂, —N(R¹⁵)C(O)R¹⁴, —N(R¹¹)C(O)OR, —N(R¹¹)C(O)N(R¹⁵)₂, —N(R¹⁵)S(O)₂(R¹⁵), —S(O)R¹¹, —S(O)₂R^{11′}, —S(O)₂N(R¹¹)₂, —NO₂, and —CN;
   (c) C₂. alkyl optionally substituted with one or more substituents independently selected from:
      halogen, —OR¹¹, —SR¹¹, —N(R¹¹)₂, —C(O)R¹¹, —C(O)—O—C₁₋₆-alkyl, —OC(O)R¹¹, —OC(O)N(R¹¹)₂—C(O)N(R¹¹)₂, —N(R¹¹)C(O)R¹¹, —N(R¹¹)C(O)OR¹², —N(R¹¹)₅)C(O)N(R¹¹)₂, —N(R¹¹)S(O)₂(R¹¹), —S(O)R¹¹)_{D}, —S(O)₂R⁵, —S(O)₂N(R¹¹)₂, —NO₂, and —CN;
      C₃₋₁₀ carbocycle and 3- to 10-membered heterocycle any of which is optionally substituted with one or more substituents independently selected from halogen, C₁₋₄haloalkyl, —OR¹¹, —SR¹¹, —N(R¹⁵)₂, —C(O)R¹⁵, —C(O)OR¹¹, —OC(O)R¹¹, —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹¹)₂, —N(R¹¹)S(O)₂(R¹¹), —S(O)R¹¹)₅, —S(O)₂R¹¹, —S(O)₂N(R¹¹)₅)₂, —NO₂, and —CN; and
   (d) C₃₋₁₀ carbocycle and 3- to 10-membered heterocycle any of which is optionally substituted with one or more substituents independently selected from:
      halogen, —OR¹¹)₅, —SR¹¹, —N(R¹¹)₂, —C(O)R¹⁵, —C(O)OR¹¹, —OC(O)R¹⁵, —OC(O)N(R¹¹)₂, —C(O)N(R¹⁵)₂, —N(R¹⁵)C(O)R¹⁵, —N(R¹⁵)C(O)OR¹², —N(R¹¹)₅)C(O)N(R¹¹)₂, —N(R^{11B})S(O)₂(R¹¹), —S(O)R¹¹, —S(O)₂R¹¹, —S(O)₂N(R¹¹)₂, —NO₂, and —CN; and
      C₁—alkyl optionally substituted with one or more substituents independently selected from halogen, —OR¹¹, —SR¹¹, —N(R¹¹), —C(O)R¹¹, —C(O)OR¹³, —OC(O)R¹¹, —OC(O)N(R¹⁴)₂, —C(O)N(R¹⁵)₂, —N(R¹⁴)C(O)R¹⁴, —N(R¹⁴)C(O)OR¹¹, —N(R¹¹)_{S})C(O)N(R¹¹), —N(R¹⁴)S(O)₂(R¹⁵), —S(O)R¹⁵, —S(O)₂R¹¹, —S(O)₂N(R¹⁵)₂, —NO₂, and —CN; or
R³ and R⁴ can come together to form a 4- to 12-membered heterocycle optionally substituted with one or more substituents independently selected from:
   halogen, —OR¹⁶, —SR^{M}, —N(R¹⁶)₂, —C(O)R¹⁵, —C(O)OR¹⁶, —OC(O)R¹⁶, —OC(O)N(R¹⁶). —C(O)N(R¹⁶)₂, —N(R¹⁶)C(O)R¹⁶, —N(R¹⁶)C(O)OR¹⁶, —N(R¹⁶)C(O)N(R¹⁶)₂—N(R¹⁶)S(O)₂(R⁶), —S(O)R¹⁶, —S(O)₂R⁶, —S(O)₂N(R⁶)₂, —NO₂. =0, and —CN; and
   C₁₋₆ alkyl optionally substituted with one or more substituents independently selected from halogen, —OR¹⁶, —SR¹⁶, —N(R^{1G})₂, —C(O)R¹⁶, —C(O)OR¹¹, —OC(O)R¹⁶, —OC(O)N(R¹⁶), —C(O)N(R¹⁶)₂, —N(R¹⁶)C(O)R¹⁶, —N(R¹⁶)C(O)OR¹⁶, —N(R¹⁶)C(O)N(R⁶)₂, —N(R¹⁶)S(Oh(R⁶), —S(O)R¹⁶, —S(O)₂R¹⁶, —S(O)₂N(R¹⁶)₂, —NO₂, ═O, and —CN;
R⁵ is selected from:
   halogen, —OR¹⁷, —SR¹⁷, —N(R¹⁷)₂, —C(O)R¹⁴, —C(O)OR¹⁷, —OC(O)R¹⁷, —OC(O)N(R¹⁷)₂. —C(O)N(R)₂, —N(R¹⁷)C(O)R¹⁷, —N(R¹⁷)C(O)OR¹⁷, —N(R¹⁷)C(O)N(R¹⁷)₂, —N(R¹⁷)S(O)₂(R¹⁷), —S(O)R¹⁷, —S(O)₂R¹⁷, —S(O)₂N(R¹⁷)₂, —NO₂, and —CN; and
   C₁₋₆ alkyl and C₂₋₆ alkenyl each of which is optionally substituted with one or more substituents independently selected from halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)S(O)(R¹⁷), —S(O)R¹¹, —S(O)₂R¹⁷, —S(O)₂N(R¹⁷)₂, —NO₂, ═O, and —CN;
R¹¹, R¹¹, R¹³, R¹², R¹⁵, R¹⁶, and R¹⁷ are independently selected at each occurrence from (I), (II) and (III):
   (I) hydrogen;
   (II) C₁₋₆ alkyl optionally substituted with one or more substituents independently selected from: halogen, —OH, —O—C₁-C₆ alkyl, —O—C₁-C₆haloalkcyl —NH₂, —NO₂, ═O, —CN, C₃₋₁₀ carbocycle and 3- to 10-membered heterocycle, wherein the C₃₋₁₀ carbocycle and 3- to 10-membered heterocycle are optionally substituted with one or more substituents selected from: halogen, —OH, C₁-C₆ haloalkyl, —O—C₁-C₆ alkyl, —O—C₁-C₆ haloalkyl —NH₂, —NO₂, -0, and —CN; and
   (III) C₃₋₁₀ carbocycle and 3- to 10-membered heterocycle, each of which is optionally substituted with one or more substituents independently selected from: halogen, —OH, C₁-C₆ haloalkyl, —O—C₁-C₆ alkyl, —O—C₁-C₆haloalkyl —NH₂, —NO₂, ═O, and —CN; and C₁₋₆ alkyl optionally substituted with one or more substituents independently selected from halogen, —OH, —O—C₁-C₆ alkyl, —O—C₁-C₆haloalkyl —NH₂, —NO₂, ═O, and —CN; and n is selected from 0, 1, 2, 3, and 4, wherein n is selected from 1, 2, 3, and 4; or at least one of R¹ or R² is hydrogen.

In some embodiments, for a compound or salt of Formula (I) or (I-a), n is selected from 1, 2, 3, and 4. In some embodiments, n is selected form 0, 1, 2, and 3. In some embodiments, n is selected from 0, 1, and 2. In some embodiments, n is selected from 0 and 1. In some embodiments, n is selected from 2, 3, and 4. In some embodiments, n is selected from 3 and 4. In some embodiments, n is selected from 1 and 2. In some embodiments, n is 4. In some embodiments, n is 3. In some embodiments, n is 2, In some embodiments, n is 1. In some embodiments, n is ₀.

In some embodiments, for a compound or salt of Formula (I) or (I-a), R³ is selected from halogen, —OR¹⁶, —SR¹⁶, —N(R¹⁶)₂, —C(O)R⁶, —C(O)OR¹⁶, —OC(O)R¹⁶, —OC(O)N(R¹⁶)₂, —C(O)N(R¹⁶)₂, —N(R¹⁶)C(O)R¹⁶, —N(R¹¹)C(O)OR¹⁶, —N(R¹⁶)C(O)N(R¹⁶)₂, —N(R¹⁶)S(O)₂(R¹⁶), —S(O)R¹⁶, —S(O)₂R¹⁶, —S(O)₂N(R¹⁶)₂, —NO₂, =O, and —CN. In some embodiments, R³ is selected from halogen, —OR¹⁶, —SR¹⁶, —N(R¹⁶)₂, —C(O)R⁶, —C(O)OR⁶, —NO₂, ═O, and —CN. In some embodiments, R is selected from C₁₋₆ alkyl and C₂₋₆ alkenyl each of which is optionally substituted with one or more substituents independently selected from halogen, —OR¹⁷, —SR¹⁷, —N(R¹⁷)₂, —C(O)R¹¹, —C(O)OR¹⁷, —OC(O)R¹⁴, —OC(O)N(R¹⁷)₂, —C(O)N(R¹⁷)₂, —N(R¹⁶)C(O)R¹¹, —N(R¹¹)C(O)OR¹¹)₇, —N(R¹⁷)C(O)N(R¹⁷)₂, —N(R¹⁷)S(O)₂(R¹⁷), —S(O)R¹⁷, —S(O)₂R¹⁷, —S(O)₂N(R¹⁷)₂, —NO₂, =O, and —CN. In some embodiments, R⁵ is selected from C₁₋₆ alkyl and C₂₋₆ alkenyl each of which is optionally substituted with one or more substituents independently selected from halogen, —OR¹⁷, —SR¹⁷, —N(R¹⁷)₂, —C(O)R¹⁷, —C(O)OR¹⁷, —NO₂, ═O, and —CN. In some embodiments, R⁵ is selected from C₁₋₃ alkyl optionally substituted with one or more substituents independently selected from halogen, —OR¹⁷, —SR¹⁷—N(R¹¹)₂, —C(O)R¹⁷, —C(O)OR¹¹, —NO₂, ═O, and —CN. In some embodiments, R¹ is selected from C₂₋₃ alkenyl each of which is optionally substituted with one or more substituents independently selected from halogen, —OR¹⁷, —SR¹⁷, —N(R¹⁷)₂, —C(O)R¹⁷, —C(O)OR¹⁷, —NO₂, =O, and —CN. In some embodiments. R⁵ is selected from halogen, —OR¹⁷, —SR¹⁷, —N(R¹⁷)₂, —C(O)R¹⁷, —NO₂, and —CN; and C₁₋₃ alkyl and C₂₋₃ alkenyl each of which is optionally substituted with one or more substituents independently selected from halogen, —OR¹⁷, —SR¹⁷, —N(R¹⁷)₂, —C(O)R¹⁷, —NO₂. =0, and —CN.

In some embodiments, for a compound or salt of Formula (I) or (I-a), R¹ is selected from chloro, fluoro, bromo, —OR¹⁷, —N(R¹⁷)₂, —CN; C₁₋₃ alkyl and C₂₋₃ alkenyl each of which is optionally substituted with one or more substituents independently selected from chloro, fluoro, —OR¹⁷, and —N(R¹⁷). In some embodiments, R⁵ is selected from chloro, fluoro, bromo, —CN, methyl, ethyl,

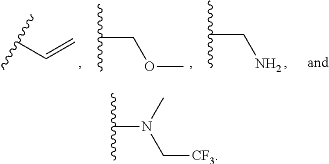

In some embodiments, R⁵ is selected from chloro, fluoro, bromo, —CN, methyl, ethyl,

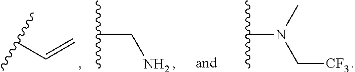

In some embodiments, R⁵ is selected from chloro, fluoro, bromo, —CN, methyl, and ethyl. In some embodiments, R⁵ is selected from

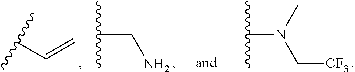

In some embodiments, R⁵ is

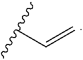 [SMILES: *C=C]

In some embodiments, R⁵ is

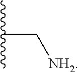 [SMILES: *CN]

In some embodiments, R⁵ is

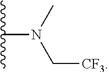 [SMILES: *N(C)CC(F)(F)F]

In some embodiments, R⁵ is

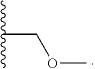 [SMILES: *COC]

In some embodiments, for the compound or salt of Formula (I) or (I-a). R is selected from halogen, —OR¹⁷, —N(R¹⁷)₂, —C(O)R¹⁷, —C(O)N(R¹⁷)₂, —N(R¹⁷)C(O)R¹¹, —N(R¹¹)C(O)OR¹⁷, —CN; C₁₋₃ alkyl and C₂₋₃ alkenyl, each of which is optionally substituted with one or more substituents independently selected from halogen, —OR¹⁷, and —N(R¹¹)₂. In some embodiments, R⁵ is selected from chloro, fluoro, —CN, methyl, ethyl, propyl, isopropyl, —CF₃, —OCH₃, —OCH₂CH₃, —OCF₃,

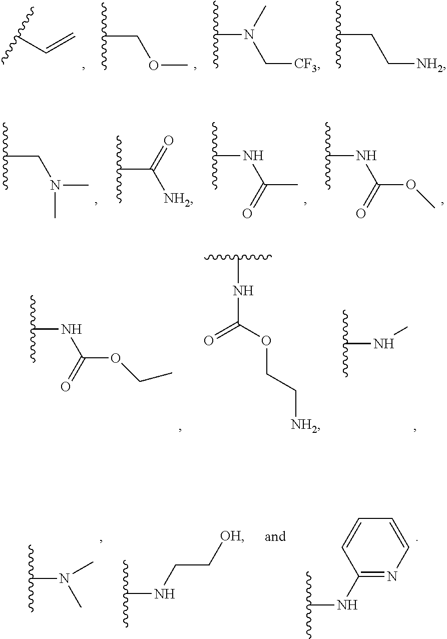

In some embodiments, R¹ is selected from chloro, fluoro, —CN, methyl, ethyl,

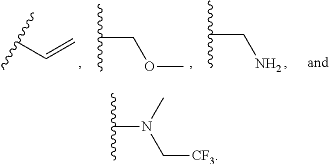

In some embodiments, for a compound or salt of Formula (I) or (I-a), A is selected from optionally substituted 5- to 6-membered heteroaryl. In some embodiments, the optionally substituted 5- to 6-membered heteroaryl of A comprises one or more heteroatoms selected from nitrogen, oxygen, sulfur, and combination thereof. In some embodiments, the optionally substituted 5- to 6-membered heteroaryl of A comprises one or more heteroatoms selected from nitrogen, oxygen, and combination thereof. In some embodiments, the optionally substituted 5- to 6-membered heteroaryl of A comprises one or more heteroatoms selected from nitrogen, sulfur, and combination thereof. In some embodiments, the optionally substituted 5- to 6-membered heteroaryl of A comprises one or more heteroatoms selected from oxygen, sulfur, and combination thereof. In some embodiments, the optionally substituted 5- to 6-membered heteroaryl of A comprises one or more nitrogen heteroatoms. In some embodiments, the optionally substituted 5- to 6-membered heteroaryl of A comprises one or more heteroatoms selected from nitrogen and oxygen.

In some embodiments, for a compound or salt of Formula (I) or (I-a), A is selected from 5 to 6-membered heteroaryl optionally substituted with one or more substituents independently selected from (i) halogen, —OR¹¹, —SR¹¹, —N(R¹¹)₂, —C(O)R¹¹, C(O)OR¹¹, —OC(O)R¹¹, —NO₂, and —CN. In some embodiments, A is selected from 5- to 6-membered heteroaryl optionally substituted with one or more substituents independently selected from (i) halogen, —OR¹¹, —SR¹¹, —N(R¹¹)₂, —C(O)R¹¹, C(O)OR¹¹, —OC(O)R¹¹, —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹¹)₂, —N(R¹¹)S(O)₂(R¹¹), —S(O)R¹¹, —S(O)₂R¹¹, —S(O)₂N(R¹¹)₂, —NO₂, ═O, and —CN.

In some embodiments, for a compound or salt of Formula (I) or (I-a), A is selected from 5 to 6-membered heteroaryl optionally substituted with one or more substituents independently selected from (ii) C₁₋₁₀ alkyl optionally substituted with one or more substituents independently selected from: halogen, —OR¹¹, —SR¹¹, —N(R¹¹)₂, —C(O)R¹¹, C(O)OR¹¹, —OC(O)R¹¹, —OC(O)N(R¹¹)₂,—C(O)N(R¹¹)₂, —N(R¹¹)C(O)R¹¹, —N(R¹¹)C(O)OR¹¹, —N(R¹¹)C(O)N(R¹¹)₂, —N(R¹¹)S(O)₂(R¹¹), —S(O)R¹¹, —S(O)₂R¹¹, —S(O)₂N(R¹¹)₂, —NO₂, ═O, and —CN. In some embodiments, A is selected from 5- to 6-membered heteroaryl optionally substituted with one or more substituents independently selected from (ii) C₁₋₁₀ alkyl optionally substituted with one or more substituents independently selected from: halogen, —OR¹¹, —SR¹¹, —N(R¹¹)₂, —C(O)R¹¹, C(O)OR¹¹, —OC(O)R¹¹, —NO₂, ═O, and —CN.

In some embodiments, for a compound or salt of Formula (I) or (I-a), A is selected from 5- to 6-membered heteroaryl optionally substituted with one or more substituents independently selected from (ii) C₁₋₁₀ alkyl optionally substituted with one or more substituents independently selected from C₃₋₁₀ carbocycle and 3- to 10-membered heterocycle each of which is optionally substituted with one or more substituents independently selected from: halogen, —OR¹¹, —SR¹¹, —N(R¹¹)₂, —C(O)R¹¹, —C(O)OR¹¹, —OC(O)R¹¹, —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹¹)₂, —N(R¹¹)S(O)₂(R1), —S(O)R¹¹, —S(O)₂R¹¹, —S(O)₂N(R¹¹)₂, —NO₂, ═O, and —CN. In some embodiments, A is selected from 5- to 6-membered heteroaryl optionally substituted with one or more substituents independently selected from (ii) C₁₋₁₀ alkyl optionally substituted with one or more substituents independently selected from C₃—,o carbocycle and 3- to 10-membered heterocycle each of which is optionally substituted with one or more substituents independently selected from: halogen, —OR¹¹, —SR¹¹, —N(R¹¹)₂, —C(O)R¹¹, —C(O)OR¹¹, —OC(O)R¹¹, —NO₂, ═O, and —CN. In some embodiments, A is selected from 5- to 6-membered heteroaryl optionally substituted with one or more substituents independently selected from (ii) C₁₋₆ alkyl optionally substituted with one or more substituents independently selected from C₃₋₆ carbocycle and 3- to 6-membered heterocycle each of which is optionally substituted with one or more substituents independently selected from: halogen, —OR¹¹, —SR¹¹, —N(R¹¹)₂, —C(O)R¹¹, —C(O)OR¹¹, —OC(O)R¹¹, —NO₂, ═O, and —CN. In some embodiments, A is selected from 5- to 6-membered heteroaryl optionally substituted with one or more substituents independently selected from (ii) C₁ 6 alkyl optionally substituted with one or more substituents independently selected from C₃₋₆ carbocycle and 4- to 6-membered saturated heterocycle each of which is optionally substituted with one or more substituents independently selected from: halogen, —OR¹¹, —SR¹¹, —N(R¹¹)₂, —C(O)R¹¹, —C(O)OR¹¹, —OC(O)R¹¹, —NO₂, ═O, and —CN. In some embodiments, A is selected from 5- to 6-membered heteroaryl optionally substituted with one or more substituents independently selected from (ii) C₁₋₆ alkyl optionally substituted with one or more substituents independently selected from C₃₋₄ carbocycle and 5- to 6-membered heteroaryl each of which is optionally substituted with one or more substituents independently selected from: halogen, —OR¹¹, —SR¹¹, —N(R¹¹)₂, —C(O)R¹¹, —C(O)OR¹¹, —OC(O)R¹¹, —NO₂. =0, and —CN. In some embodiments, A is selected from 5- to 6-membered heteroaryl optionally substituted with one or more substituents independently selected from (ii) C₁—alkyl optionally substituted with one or more substituents independently selected from C₃₋₆ carbocycle, 4- to 6-membered saturated heterocycle, and 5- to 6-membered heteroaryl each of which is optionally substituted with one or more substituents independently selected from: halogen, —OR¹¹, —SR¹¹, —N(R¹¹)₂, —C(O)R¹¹, —C(O)OR¹¹, —OC(O)R¹¹, —NO₂, ═O, and —CN.

In some embodiments, for a compound or salt of Formula (I) or (I-a), A is selected from 5- to 6-membered heteroaryl optionally substituted with one or more substituents independently selected from (iii) 3- to 10-membered heterocycle optionally substituted with one or more substituents independently selected from: halogen, —OR¹¹, —SR¹¹, —N(R¹¹)₂, —C(O)R¹¹, —C(O)OR¹¹, —OC(O)R¹¹, —OC(O)N(R¹¹)₂, —C(O)N(R¹¹)₂, —N(R¹¹)C(O)R¹¹, —N(R¹¹)C(O)OR, —N(R¹¹)C(O)N(R¹¹)₂, —N(R¹¹)S(O)₂(R¹¹), —S(O)R¹¹, —S(O)₂R¹¹, —S(O)₂N(R¹¹)₂, —NO₂, ═O, —CN. In some embodiments, A is selected from 5- to 6-membered heteroaryl optionally substituted with one or more substituents independently selected from (iii) 3- to 10-membered heterocycle optionally substituted with one or more substituents independently selected from: halogen, —OR¹¹, —SR¹¹, —N(R¹¹)₂, —C(O)R¹¹, —C(O)OR¹¹, —OC(O)R¹¹, —NO₂, ═O, and —CN. In some embodiments, A is selected from 5- to 6-membered heteroaryl optionally substituted with one or more substituents independently selected from (iii) 3- to 6-membered heterocycle optionally substituted with one or more substituents independently selected from: halogen, —OR¹¹, —SR¹¹, —N(R¹¹)₂, —C(O)R¹¹, —C(O)OR¹¹, —OC(O)R¹¹,—NO₂, ═O, and —CN.

In some embodiments, for a compound or salt of Formula (I) or (I-a). A is selected from 5- to 6-membered heteroaryl optionally substituted with one or more substituents independently selected from (iii) 3- to 10-membered heterocycle optionally substituted with one or more substituents independently selected from Ci_6 alkyl optionally substituted with one or more substituents independently selected from halogen, —OR¹¹, —SR¹¹, —N(R¹¹)₂, —C(O)R¹¹, —C(O)OR¹¹, —OC(O)R¹¹, —OC(O)N(R¹¹)₂, —C(O)N(R¹¹)₂, —N(R¹¹)C(O)R¹¹, —N(R¹¹)C(O)OR¹¹,—N(R¹¹)C(O)N(R¹¹)₂, —N(R¹¹)S(O)₂(R¹¹), —S(O)R¹¹, —S(O)₂R¹¹, —S(O)₂N(R¹¹)₂, —NO₂, ═O, and —CN. In some embodiments, A is selected from 5- to 6-membered heteroaryl optionally substituted with one or more substituents independently selected from (iii) 3- to 10-membered heterocycle optionally substituted with one or more substituents independently selected from C₁—alkyl optionally substituted with one or more substituents independently selected from halogen, —OR¹¹, —SR¹¹, —N(R¹¹)₂, —C(O)R¹¹, —C(O)OR¹¹, —OC(O)R¹¹, —NO₂, =O, and —CN. In some embodiments, A is selected from 5- to 6-membered heteroaryl optionally substituted with one or more substituents independently selected from (iii) 3- to 6-membered heterocycle optionally substituted with C—alkyl optionally substituted with one or more substituents independently selected from halogen, —OR¹¹, —SR¹¹, —N(R¹¹)₂, —C(O)R¹¹, —C(O)OR¹¹, —OC(O)R¹¹, —NO₂, ═O, and —CN.

In some embodiments, for a compound or salt of Formula (I) or (I-a), A is selected from 5- to 6-membered heteroaryl optionally substituted with one or more substituents independently selected from:
(ii) C₁₋₁₀ alkyl optionally substituted with one or more substituents independently selected from:
   halogen, —OR¹¹, —N(R¹¹)₂, —C(O)R¹¹, —C(O)OR¹¹, —NO₂, ═O, —CN; and C₃₋₁₀ carbocycle and 3- to 10-membered heterocycle each of which is optionally substituted with one or more substituents independently selected from halogen, —OR¹¹, —N(R¹¹)₂, —C(O)R¹¹, —C(O)OR¹¹)₁, —NO₂, ═O. —CN; and
(iii) 3- to 10 membered heterocycle optionally substituted with one or more substituents independently selected from:
   halogen, —OR¹¹, —N(R¹¹)₂, —C(O)R¹¹, —C(O)OR¹¹, —NO₂, ═O, —CN; and C₁₋₆ alkyl optionally substituted with one or more substituents independently selected from halogen, —OR¹¹, —N(R¹¹)₂, —C(O)R¹¹, —C(O)OR¹¹, —NO₂, ═O, and —CN.

In some embodiments, for a compound or salt of Formula (I) or (I-a), A is selected from an optionally substituted 5- membered heteroaryl. In some embodiments, A is selected from pyrazole, oxazole, isoxazole, thiazole, isothiazole, pyrrole, furan, thiophene, imidazole, triazole, tetrazole, and pyridine, any of which is optionally substituted. In some embodiments, A is selected from pyrazole, oxazole, isoxazole, thiazole, isothiazole, pyrrole, furan, thiophene, imidazole, triazole, and tetrazole, any of which is optionally substituted with one or more substituents independently selected from:
(ii) C₁₋₁₀ alkyl optionally substituted with one or more substituents independently selected from:
   halogen, —OR¹¹, —N(R¹¹)₂, —C(O)R¹¹, —C(O)OR¹¹, —NO₂, ═O, —CN; and C₃₋₁₀ carbocycle and 3- to 10-membered heterocycle each of which is optionally substituted with one or more substituents independently selected from halogen, —OR¹¹, —N(R¹¹)₂, —C(O)R¹¹, —C(O)OR¹¹, —NO₂, =O, —CN; and
(iii) 3- to 10 membered heterocycle optionally substituted with one or more substituents independently selected from:
   halogen, —OR¹¹, —N(R¹¹)₂, —C(O)R¹¹, —C(O)ORR¹¹, —NO₂, ═O, —CN; and C₁₋₆ alkyl optionally substituted with one or more substituents independently selected from halogen, —OR¹¹, —N(R¹¹)₂, —C(O)R¹¹, —C(O)OR¹¹, —NO₂, ═O, and —CN.

In some embodiments, for a compound or salt of Formula (I) or (I-a), A is selected from:

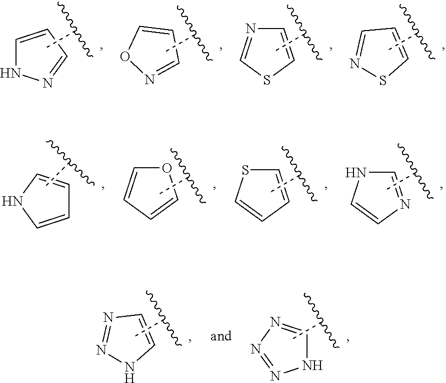

any of which are optionally substituted with one or more substituents independently selected from:
(ii) C₁₋₁₀ alkyl optionally substituted with one or more substituents independently selected from:
   halogen, —OR¹¹, —N(R¹¹)₂, —C(O)R¹¹, —C(O)OR¹, —NO₂. =0, —CN; and C₃ to carbocycle and 3- to 10-membered heterocycle each of which is optionally substituted with one or more substituents independently selected from halogen, —OR¹¹, —N(R¹¹)₂, —C(O)R¹¹, —C(O)OR¹¹, —NO₂, ═O, —CN; and
(iii) 3- to 10 membered heterocycle optionally substituted with one or more substituents independently selected from:
   halogen, —OR¹¹, —N(R¹¹)₂, —C(O)R¹¹, —C(O)OR¹¹, —NO₂, ═O, —CN; and C₁₋₆ alkyl optionally substituted with one or more substituents independently selected from halogen, —OR¹¹, —N(R¹¹)₂, —C(O)R¹¹, —C(O)OR¹¹, —NO₂, ═O, and —CN.

In some embodiments, for a compound or salt of Formula (I) or (I-a). A is selected from optionally substituted pyrazole and optionally substituted isoxazole. In some embodiments, A is

selected from:

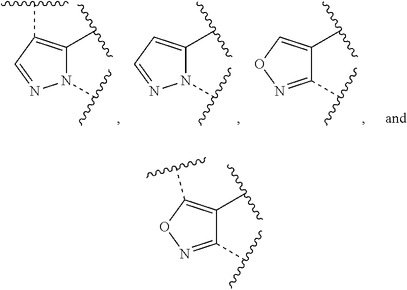

each of which is optionally substituted; and wherein

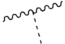

denotes the connection to the optional substituent and

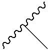

denotes the connection of A to the remainder of Formula I. In some embodiments, A is selected from:

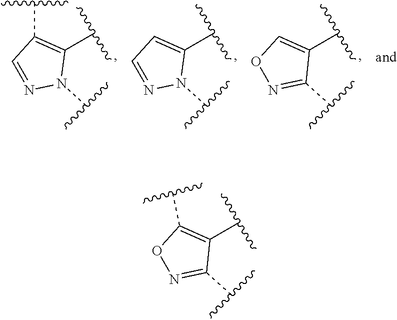

each of which is optionally substituted;

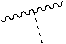

denotes a first connection; and

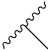

denotes a second connection, wherein the first connection is from A to the optional substituents and the second connection represents the bond between A and the structure of Formula (I) or Formula (I-a).

In some embodiments, for a compound or salt of Formula (I) or (I-a), the 5- to 6-membered heterocycle of A is substituted with one or more substituents. In some embodiments, the substituents on the 5- to 6-membered heterocycle of A are independently selected from (i), (ii), and (iii):
(i) halogen, —OR¹¹, —SR¹¹, —N(R¹¹)₂, —C(O)R¹¹, —NO₂, ═O, and —CN;
(ii) C₁-6 alkyl optionally substituted with one or more substituents independently selected from:
   halogen, —OR¹¹, —SR¹¹, —N(R¹¹)₂, —C(O)R¹¹, —NO₂, ═O, —CN; and C₃₋₁₀ carbocycle and 3- to 10-membered heterocycle each of which is optionally substituted with one or more substituents independently selected from; halogen, —OR¹¹, —SR¹¹, —N(R¹¹)₂, —C(O)R¹¹, —NO₂, ═O, and —CN; and
(iii) 3- to 10-membered heterocycle optionally substituted with one or more substituents independently selected from:
   halogen, —OR¹¹, —SR¹¹, —N(R¹¹)₂, —C(O)R¹¹, —NO₂, ═O, —CN; and C₁—alkyl optionally substituted with halogen.

In some embodiments, for a compound or salt of Formula (I) or (I-a), the 5- to 6-membered heterocycle of A is substituted with one or more substituents. In some embodiments, the substituents on the 5- to 6-membered heterocycle of A are independently selected from (i) halogen, —OR¹¹, —SR¹¹, —N(R¹¹)₂, —C(O)R¹¹, —NO₂, =O, and —CN.

In some embodiments, for a compound or salt of Formula (I) or (I-a), the 5- to 6-membered heterocycle of A is substituted with one or more substituents. In some embodiments, the substituents on the 5- to 6-membered heterocycle of A are independently selected from (ii) C₁₋₆ alkyl optionally substituted with one or more substituents independently selected from:
halogen, —OR¹¹, —SR¹¹, —N(R¹¹)₂, —C(O)R¹¹, —NO₂, ═O, —CN;
C₃₋₁₀ carbocycle and 3- to 10-membered heterocycle each of which is optionally substituted with one or more substituents selected from: halogen, —OR¹¹, —SR¹¹, —N(R¹¹)₂, —C(O)R¹¹, —NO₂, =O, and —CN. In some embodiments, the substituents on the 5- to 6-membered heterocycle of A are independently selected from (ii) C₁₋₆ alkyl optionally substituted with one or more substituents independently selected from: C₃₋₁₀ carbocycle optionally substituted with one or more substituents independently selected from: halogen, —OR¹¹, —SR¹¹, —N(R¹¹)₂, —C(O)R¹¹, —NO₂, ═O, and —CN. In some embodiments, the substituents on the 5- to 6-membered heterocycle of A are independently selected from (ii) C₁₋₆ alkyl optionally substituted with one or more substituents independently selected from: 3- to 10-membered heterocycle optionally substituted with one or more substituents independently selected from: halogen, —OR¹¹, —SR¹¹, —N(R¹¹)₂, —C(O)R¹¹, —NO₂, ═O, and —CN. In some embodiments, the substituents on the 5- to 6-membered heterocycle of A are independently selected from (ii) C₁₋₆ alkyl optionally substituted with one or more substituents independently selected from: 3- to 10-membered saturated heterocycle optionally substituted with one or more substituents independently selected from: halogen, —OR¹¹, —SR¹¹, —N(R¹¹)₂, —C(O)R¹¹, —NO₂, =O, and —CN. In some embodiments, the substituents on the 5- to 6-membered heterocycle of A are independently selected from (ii) C₁₋₆ alkyl optionally substituted with one or more substituents independently selected from: 3- to 6-membered saturated heterocycle optionally substituted with one or more substituents independently selected from: halogen, —OR¹¹, —SR¹¹, —N(R¹¹)₂, —C(O)R¹¹, —NO₂, ═O, and —CN. In some embodiments, the substituents on the 5- to 6-membered heterocycle of A are independently selected from (ii)C-₆ alkyl optionally substituted with one or more substituents independently selected from azetidine, piperidine, piperazine, morpholine, and thiomorpholine, any of which is optionally substituted with one or more substituents independently selected from: halogen, —OR¹¹, —SR¹¹, —N(R¹¹)₂, —C(O)R¹¹, —NO₂, ═O, and —CN.

In some embodiments, for a compound or salt of Formula (I) or (I-a), the 5- to 6-membered heterocycle of A is substituted with one or more substituents. In some embodiments, the substituents on the 5- to 6-membered heterocycle of A are independently selected from (iii) 3- to 10 membered heterocycle optionally substituted with one or more substituents independently selected from halogen, —OR¹¹, —SR¹¹, —N(R¹¹)₂, —C(O)R¹¹, —NO₂, ═O, —CN and C₁₋₆ haloalkyl. In some embodiments, the substituents on the 5- to 6-membered heterocycle of A are independently selected from (iii) 3- to 10 membered saturated heterocycle optionally substituted with one or more substituents independently selected from halogen, —OR¹¹, —SR¹¹, —N(R¹¹)₂, —C(O)R¹¹, —NO₂, ═O, —CN and Ci, haloalkyl. In some embodiments, the substituents on the 5- to 6-membered heterocycle of A are independently selected from (iii) 3- to 6 membered saturated heterocycle optionally substituted with one or more substituents independently selected from halogen, —OR¹¹, —SR¹¹, —N(R¹¹)₂, —C(O)R¹¹, —NO₂, =O, —CN and C₁₋₆haloalkyl.

In some embodiments, for a compound or salt of Formula (I) or (I-a), the 5- to 6-membered heterocycle of A is substituted with one or more substituents. In some embodiments, the substituents on the 5- to 6-membered heterocycle of A are independently selected from (ii) and (iii):
(ii) C₁₋₆ alkyl optionally substituted with one or more substituents independently selected from:
   halogen, —OR¹¹, —SR¹¹, —N(R¹¹)₂, —C(O)R¹¹, —NO₂, =O, —CN; and C₃₋₁₀ carbocycle and 3- to 10-membered heterocycle each of which is optionally substituted with one or more substituents selected from: halogen, —OR¹¹, —SR¹¹¹, —N(R¹¹)₂, —C(O)R¹¹, —NO₂, ═O, and —CN; and
(iii) 3- to 10-membered heterocycle optionally substituted with one or more substituents independently selected from:
   halogen, —OR¹¹, —SR¹¹, —N(R¹¹)₂, —C(O)R¹¹, —NO₂, ═O, —CN; and C₁₋₆ alkl optionally substituted with halogen.

In some embodiments, for a compound or salt of Formula (I) or (I-a), the 5- to 6-membered heterocycle of A is substituted with one or more substituents. In some embodiments, the substituents on the 5- to 6-membered heterocycle of A are independently selected from (ii) and (iii):
(ii) C₁₋₆ alkyl optionally substituted with one or more substituents independently selected from:
   halogen, —OR¹¹, —SR¹¹, —N(R¹¹)₂, —C(O)R¹¹, —NO₂, ═O, —CN; and C₃₋₁₀ carbocycle and 3- to 10-membered heterocycle each of which is optionally substituted with one or more substituents selected from: halogen, —OR¹¹, —SR¹¹, —N(R¹¹)₂, —C(O)R¹¹, —NO₂, ═O, and —CN; and
(iii) 3- to 6-membered heterocycle optionally substituted with one or more substituents independently selected from:
   halogen, —OR¹¹, —SR¹¹, —N(R¹¹)₂, —C(O)R¹¹, —NO₂, ═O, —CN; and C₁₋₆ alkyl optionally substituted with halogen.

In some embodiments, for a compound or salt of Formula (I) or (I-a), the substituents on the 5- to 6-membered heterocycle of A are independently selected from: fluorine, methyl, ethyl,

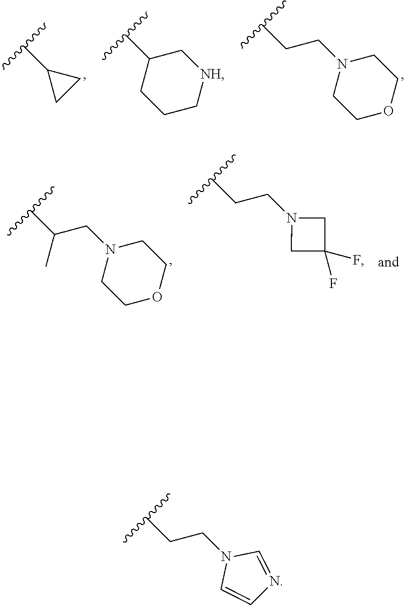

In some embodiments, the 5- to 6-membered heteroaryl of A is independently selected from pyrazole, isoxazole, oxadiazole, and pyridine, each of which is optionally substituted with substituents selected from: fluorine, methyl, ethyl, propyl, isopropyl, ,

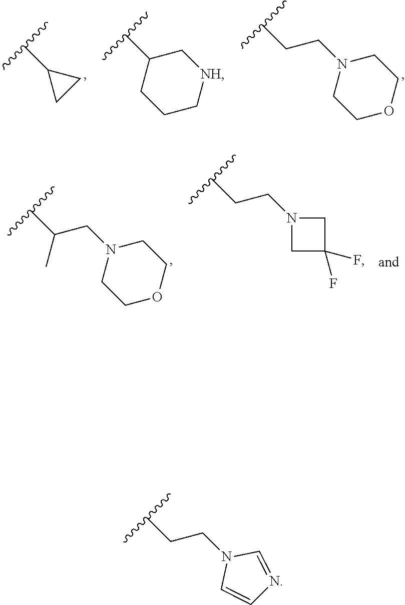

In some embodiments, for a compound or salt of Formula (I) or (I-a), the substituents on the 5- to 6-membered heterocycle of A are independently selected from: fluorine, methyl, ethyl,

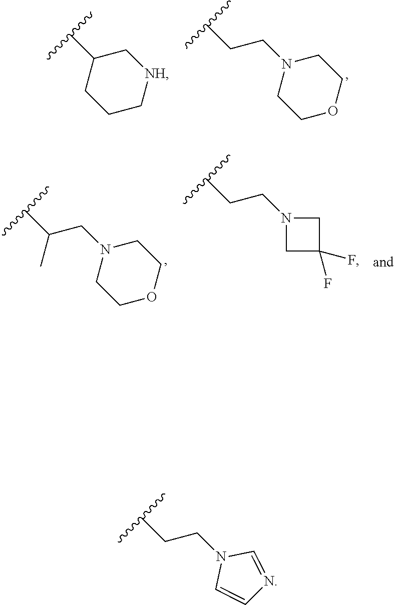

isopropyl, embodiments, the 5- to 6-membered heteroaryl of A is independently selected from pyrazole, isoxazole, and pyridine, each of which is optionally substituted with substituents selected from; fluorine, methyl, ethyl, propyl, isopropyl,

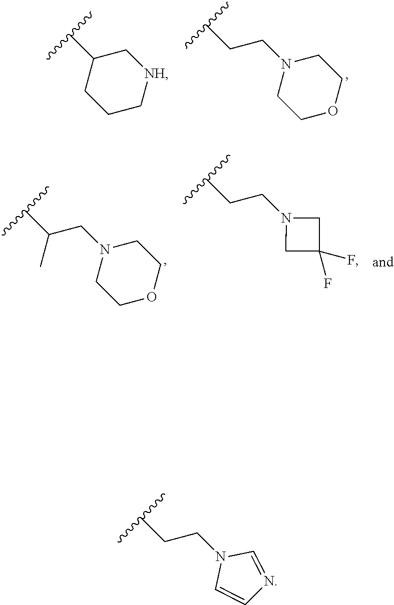

In some embodiments, for a compound or salt of Formula (I) or (I-a), A is selected from:

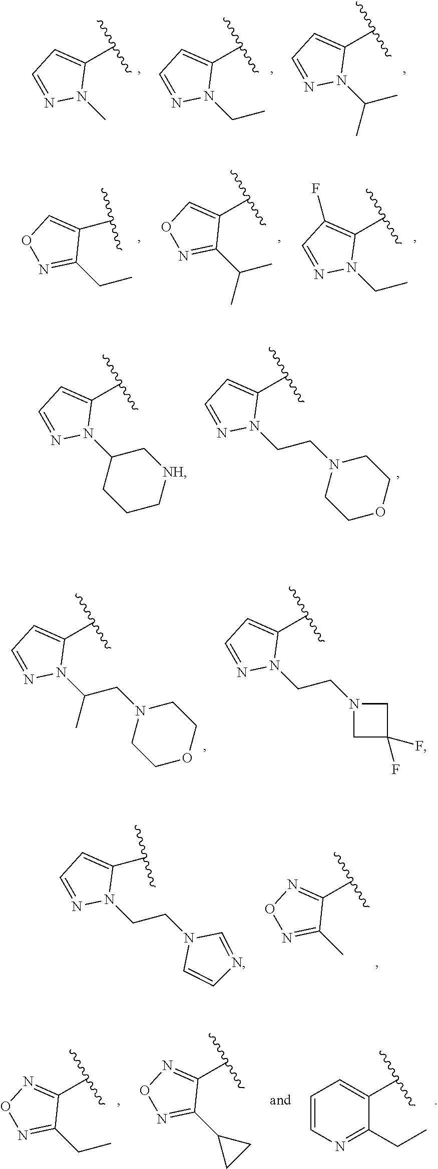

In some embodiments, for a compound or salt of Formula (I) or (I-a), A is selected from:

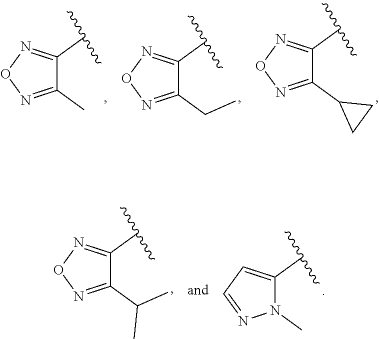

In some embodiments, for a compound or salt of Formula (I) or (I-a), A is selected from:

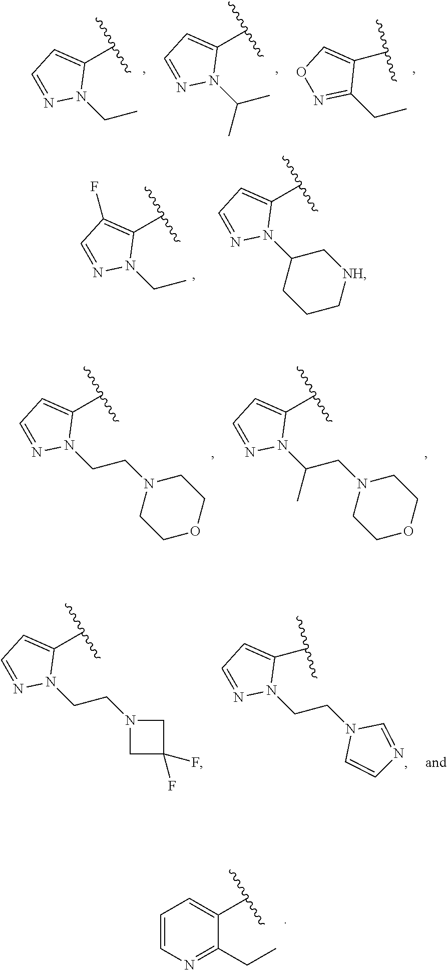

In some embodiments, for a compound or salt of Formula (I) or (I-a), B is selected from —CH(R^{A})(R^{B}) and optionally substituted C₃—.o carbocycle. In some embodiments, B is —CH(R^{A})(R^{B}) and each of R^{A} and R^{B} are independently selected from:
C₁₋₆ alkyl optionally substituted with one or more substituents independently selected from halogen, —OR¹³, —SR¹³, —N(R¹¹)₂, —C(O)R¹¹, —C(O)OR¹³, —OC(O)R¹³, —OC(O)N(R¹¹)₂, —C(O)N(R¹¹)₂, —N(R¹³)C(O)R¹³, —N(R¹¹)C(O)OR¹², —N(R¹³)C(O)N(R¹¹)₂, —N(R¹¹)₂S(O)₂(R¹¹), —S(O)R¹¹, —S(O)₂RD, —S(O)₂N(R¹¹)₂, —NO₂, and —CN; and
C₃₋₁₀ carbocycle optionally substituted with one or more substituents independently selected from halogen, —OR¹³, —SR¹², —N(R¹¹)₂, —C(O)R¹¹, —C(O)OR¹¹, —OC(O)R¹¹, —OC(O)N(R¹³)₂,—C(O)N(R¹³)₂, —N(R¹³)C(O)R¹¹, —N(R¹¹)C(O)OR¹¹, —N(R¹¹)C(O)N(R¹³)₂, —N(R¹³)S(O)₂(R³), —S(O)R¹¹, —S(O)₂R¹¹, —S(O)₂N(R¹¹)₂, —NO₂, and —CN.

In some embodiments, for a compound or salt of Formula (I) or (I-a). B is —CH(R^{A})(R^{B}) and each of R^{A} and R^{B} are independently selected from:
C₁₋₆ alkyl optionally substituted with one or more substituents independently selected from halogen, —OR¹³, —SR¹³, —N(R¹¹)₂, —C(O)R¹¹, —C(O)OR¹¹, —OC(O)R¹¹, —NO₂, and —CN; and
C₃₋₁₀ carbocycle optionally substituted with one or more substituents independently selected from halogen, —OR¹³, —SR¹¹, —N(R¹¹)₂, —C(O)R¹², —C(O)OR¹², —OC(O)R¹¹, —NO₂, and —CN.

In some embodiments, for a compound or salt of Formula (I) or (I-a), B is selected from —CH(R^{A})(R¹¹) and each of R¹¹ and R^{B} are independently selected from:
C₁₋₆ alkyl optionally substituted with one or more substituents independently selected from halogen, —OR¹², —SR¹¹, —N(R¹)₂, —C(O)R¹³, —NO₂, and —CN; and
C₃₋₆ carbocycle optionally substituted with one or more substituents independently selected from halogen, —OR¹³, —SR¹³, —N(R¹¹)₂, —C(O)R¹¹, —NO₂, and —CN.

In some embodiments, for a compound or salt of Formula (I) or (I-a), B is selected from —CH(R^{A})(R^{B}) and each of R^{A} and R^{B} are independently selected from:
methyl, ethyl, propyl, and isopropyl, each of which is optionally substituted with one or more substituents independently selected from halogen, —OR¹¹, —SR¹², —N(R¹¹), —C(O)R³, —NO₂, and —CN; and
C₃₋₆ saturated carbocycle optionally substituted with one or more substituents independently selected from halogen, —OR¹², —SR¹¹, —N(R¹¹)₂, —C(O)R¹², —NO₂, and —CN.

In some embodiments, for a compound or salt of Formula (I) or (I-a), B is selected from —CH(R^{A})(R^{B}) and each of R^{A} and R^{B} are independently selected from:
methyl, ethyl, propyl, and isopropyl, each of which is optionally substituted with one or more substituents independently selected from halogen, —OR³, —SR¹³, —N(R¹³), —C(O)R¹³, —NO₂, and —CN; and
C₆ carbocycle optionally substituted with one or more substituents independently selected from halogen, —OR¹³, —SR¹³, —N(R¹³)₂, —C(O)R³, —NO₂, and —CN.

In some embodiments, for a compound or salt of Formula (I) or (I-a), B is selected from —CH(R^{A})(R^{B}) and each of R^{A} and R^{B} are independently selected from:
methyl, ethyl, propyl, and isopropyl, each of which is optionally substituted with one or more substituents independently selected from halogen, —OR³, —SR¹³, —N(R¹³), —C(O)R¹³, —NO₂, and —CN; and
cyclopropyl, cyclobutyl, cyclopentyl, cyclohexyl, and phenyl, each of which is optionally substituted with one or more substituents independently selected from halogen, —OR³, —SR¹¹, —N(R¹³), —C(O)R¹¹)₃, —NO₂, and —CN.

In some embodiments, for a compound or salt of Formula (I) or (I-a), R^{A} and R^{A} are each independently selected from: ethyl, propyl, isopropyl, cyclopropyl, cyclobutyl, cyclopentyl, and phenyl, any of which is optionally substituted. In some embodiments, B is selected from:

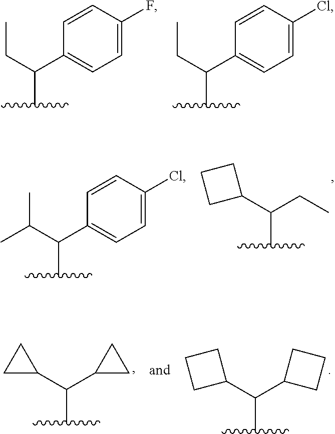

In some embodiments, for a compound or salt of Formula (I) or (I-a), B is optionally substituted C₃₋₁₀ carbocycle. In some embodiments, B is an optionally substituted saturated C₃₋₁₀ carbocycle. In some embodiments, B is an optionally substituted unsaturated C₃₋₁₀ carbocycle. In some embodiments, B is selected from C₃ carbocycle, C₄ carbocycle, C₅ carbocycle, C₆ carbocycle, C₇ carbocycle, C₈ carbocycle, C₉ carbocycle, C₁₀ carbocycle, any of which is optionally substituted. In some embodiments, B is selected from C₄₋₁₀ carbocycle, C₅₋₁₀ carbocycle, C₆₋₁₀ carbocycle, C₇₋₁₀ carbocycle, C₈₋₁₀ carbocycle, and C₉₋₁₀ carbocycle, any of which is optionally substituted.

In some embodiments, for a compound or salt of Formula (I) or (I-a), B is an optionally substituted C₅₋₈ cycloalkyl or optionally substituted C₇₋₁₀ bicyclic carbocycle. In some embodiments, B is selected from C.s cycloalkyl and C₇₋₁₀ bicyclic carbocycle and of which is optionally substituted with one or more substituents independently selected from halogen, —OR¹², —SR¹², —N(RZb, —C(O)R¹¹, —C(O)OR¹¹, —OC(O)R¹², —NO₂, ═O, —CN, —Ci.haloalkyl, and —C₁₋₄ alkoxy.

In some embodiments, B is an optionally substituted C₆₋₁₀ carbocycle selected from cyclohexyl, cycloheptyl, cyclooctyl, spiro [2.5] octanyl, and indanyl any of which are optionally substituted. In some embodiments, B is an optionally substituted C₆₋₁₀ carbocycle selected from cyclohexyl, cycloheptyl, cyclooctyl, spiro [2.5] octanyl, and indanyl any of which are optionally substituted with one or more substituents independently selected from halogen, —OR¹¹, —SR², —N(R¹¹)₂, —C(O)R^{11B}, —C(O)OR^{11B}, —OC(O)R¹², —NO₂, ═O, —CN, —C₁₋₆ haloalkyl, and —C embodiments, B is selected from

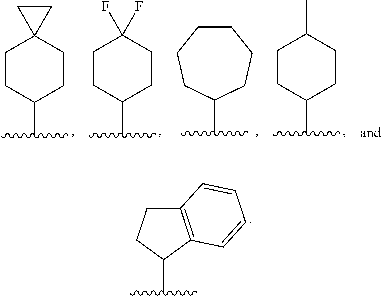

In some embodiments, for a compound or salt of Formula (I) or (I-a), B is selected from:

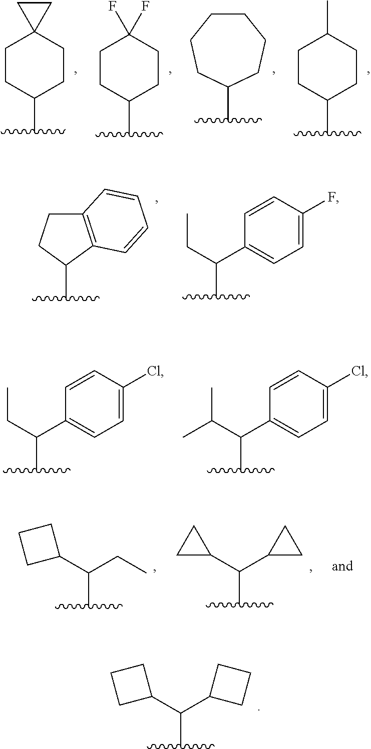

In some embodiments, for a compound or salt of Formula (I) or (I-a), R¹ and R² are each independently selected from halogen, —OR¹⁴, —SR¹⁴, —N(R¹⁴)₂, —C(O)R¹⁴, —C(O)OR¹⁴, —OC(O)R¹⁴, —OC(O)N(R¹⁴)₂, —C(O)N(R¹⁴)₂, —N(R¹⁴)C(O)R¹⁴, —N(R¹⁴)C(O)OR¹⁴, —N(R¹⁴)C(O)N(R¹⁴)₂, —N(R¹⁴)S(O)₂(R¹⁴), —S(O)R¹⁴, —S(O)₂R¹⁴, —S(O)₂N(R¹⁴)₂, —NO₂, and —CN. In some embodiments, R¹ and R² are each independently selected from halogen, —OR¹⁴, —SR¹⁴—N(R¹⁴)₂, —C(O)R¹⁴, —C(O)OR¹⁴, —OC(O)R¹⁴, —NO₂, and —CN.

In some embodiments, for a compound or salt of Formula (I) or (I-a). R¹ and R² are each Ci-6 alkyl optionally substituted with one or more substituents independently selected from halogen, —OR¹⁴, —SR¹⁴, —N(R¹⁴)₂, —C(O)R¹⁴, —C(O)OR¹⁴, —OC(O)R¹⁴, —OC(O)N(R¹⁴)₂, —C(O)N(R¹⁴)₂, —N(R¹⁴)C(O)R¹⁴, —N(R¹¹)C(O)OR¹⁴, —N(R¹⁴)C(O)N(R¹⁴)₂, —N(R¹⁴)S(O)₂(R¹⁴), —S(O)R¹⁴, —S(O)₂R¹⁴, —S(O)₂N(R¹⁴)₂, —NO₂, and —CN. In some embodiments, R¹ and R² are each Ci-_alkyl optionally substituted with one or more substituents independently selected from halogen, —OR^{1′}, —SR¹⁴, —N(R¹⁴r, —C(O)R¹⁴, —C(O)OR¹⁴, —OC(O)R¹⁴, —NO₂, and —CN. In some embodiments. R¹ and R² are each independently C-3 alkyl optionally substituted with a substituent independently selected from halogen, —OR¹⁴, —SR¹⁴, —N(R¹⁴)₂, —C(O)R¹⁴, —C(O)OR¹⁴, —OC(O)R¹⁴, —NO₂, and —CN.

In some embodiments, for a compound or salt of Formula (I) or (I-a), R¹ and R² are each
hydrogen. In some embodiments, R¹ and R² are each independently selected from:
hydrogen, halogen, —OR¹⁴, —NO₂, and —CN; and
C₁₋₆ alkyl optionally substituted with one or more substituents independently selected from halogen, —OR¹⁴, —SR¹⁴, —N(R¹⁴), —C(O)R¹⁴, —C(O)OR¹⁴, —OC(O)R¹⁴, —OC(O)N(R¹⁴)₂, —C(O)N(R¹⁴)₂, —N(R¹⁴)C(O)R¹⁴, —N(R¹⁴)C(O)OR¹⁴, —N(R¹⁴)C(O)N(R¹⁴)₂, —N(R¹⁴)S(O)₂(R¹⁴), —S(O)R¹⁴, —S(O)₂R¹⁴, —S(O)₂N(R¹¹)₂, —NO₂, and —CN.

In some embodiments, for a compound or salt of Formula (I) or (I-a), R¹ and R² are each hydrogen. In some embodiments, R¹ and R² are each independently selected from:
hydrogen, halogen, —OR¹⁴, —NO₂, and —CN; and
C₁₋₆ alkyl optionally substituted with one or more substituents independently selected from halogen, —OR¹⁴, —SR¹⁴, —N(R¹⁴), —C(O)R¹⁴, —C(O)OR¹⁴, —OC(O)R¹², —OC(O)N(R¹⁴), —C(O)N(R¹⁴)₂—N(R¹⁴)C(O)R¹⁴, —N(R¹⁴)C(O)OR¹⁴, —N(R¹⁴)C(O)N(R¹⁴)₂, —N(R¹⁴)S(O)₂(R¹⁴), —S(O)R¹⁴, —S(O)₂R¹⁴, —S(O)₂N(R¹⁴)₂, —NO₂, and —CN.

In some embodiments, for the compound or salt of Formula (I) or (I-a), R¹ and R² are each independently selected from: hydrogen,

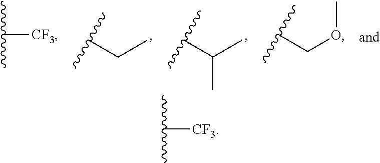

In some embodiments, R¹ and R² are each independently selected from: hydrogen,

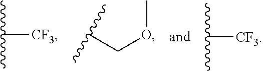

In some embodiments, R¹ and R² are each independently selected from:

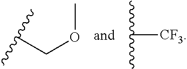

In some embodiments, R¹ and R² are each independently selected from: hydrogen and

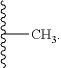

In some embodiments, R¹ and R² are each independently selected from: hydrogen,

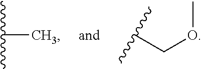

In some embodiments, R¹ and R² are each selected from hydrogen,

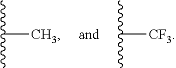

In some embodiments, R¹ and R² are each independently selected from: hydrogen,

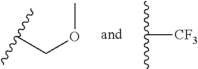

In some embodiments, R¹ and R² are selected from different substituents. In some embodiments, one of R¹ and R², is hydrogen. In some embodiments, one of R¹ and R², is C₁₋₃ alkyl. In some embodiments, one of R¹ and R² is selected from methyl, ethyl, and propyl. In some embodiments, one of R¹ and R² is methyl. In some embodiments, R¹ is hydrogen; and R² is selected from: methyl, ethyl, propyl, isopropyl and butyl, each of which is optionally substituted with one or more substituents independently selected from chloro, fluoro, bromo, and —OR¹⁴. In some embodiments, R¹ is hydrogen; and R² is selected from: methyl, ethyl, propyl, and butyl, each of which is optionally substituted with one or more substituents independently selected from chloro, fluoro, bromo, and —OR. In some embodiments, R¹ and R² are each independently selected from; hydrogen, and

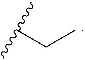 [SMILES: *CC]

In some embodiments, R¹ and R² are each independently selected from; hydrogen, and

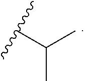 [SMILES: *C(C)C]

In some embodiments, for a compound or salt of Formula (I) or (I-a). R³ and R⁴ are each independently selected from hydrogen. In some embodiments, one of R³ and R⁴ is selected from hydrogen. In some embodiments, one of R¹ and R¹⁴ is selected from (a). In some embodiments, one of R³ and R⁴ is selected from (b). In some embodiments, one of R³ and R⁴ is selected from (c). In some embodiments, one of R³ and R⁴ is selected from (d).

In some embodiments, for a compound or salt of Formula (I) or (I-a), each of R³ and R⁴ is selected from (c). In some embodiments, each of R³ and R⁴ is selected from C₂_alkyl optionally substituted with one or more substituents independently selected from: halogen, —OR¹⁵, —SR⁵, —N(R¹¹)₂, —C(O)R¹¹, —C(O)—O—C₁₋₆ₐ alkyl, —OC(O)R¹¹, —OC(O)N(R¹⁵)₂, —C(O)N(R¹⁴)₂,—N(R¹¹)_{S})C(O)R¹¹, —N(R¹¹)C(O)OR¹¹, —N(R¹¹)C(O)N(Rts)₂, —N(R¹¹)S(O)₂(R¹¹), —S(O)R¹⁵, —S(O)₂R¹¹, —S(O)₂N(R¹¹)₂, —NO₂, and —CN. In some embodiments, each of R³ and R⁴ is selected from C₂₋₆ alkyl optionally substituted with one or more substituents independently selected from: C₃₋₁₀ carbocycle and 3- to 10-membered heterocycle any of which is optionally substituted with one or more substituents independently selected from halogen, C₁₋₆ haloalkyl, C₁₋₆ alkoxy, —OR¹⁵, —SR¹⁵, —N(R¹¹)₂, —C(O)R¹¹)_{S}, —C(O)OR¹¹, —OC(O)R¹¹, —OC(O)N(R¹¹)₂, —C(O)N(R¹¹)₂, —N(R¹⁵)C(O)R¹⁵, —N(R^{1S})C—(O)OR¹¹, —N(R¹¹)C(O)N(R¹¹)₂, —N(R¹⁵)S(O)₂(R¹⁵), —S(O)R⁵, —S(O)₂R⁵, —S(O)₂N(R⁵)₂, —NO₂, and —CN. In some embodiments, each of R³ and R⁴ is selected from C₂₋₆ alkyl optionally substituted with one or more substituents independently selected from: halogen, —OR¹⁵, —SR¹⁵, —N(R¹⁵)₂, —C(O)R¹⁵, —OC(O)R¹⁵)¹⁵, —OC(O)N(R¹⁵)₂, —C(O)N(R¹⁵)₂, —NO₂, and —CN. In some embodiments, each of R³ and R⁴

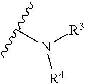

is represented by:

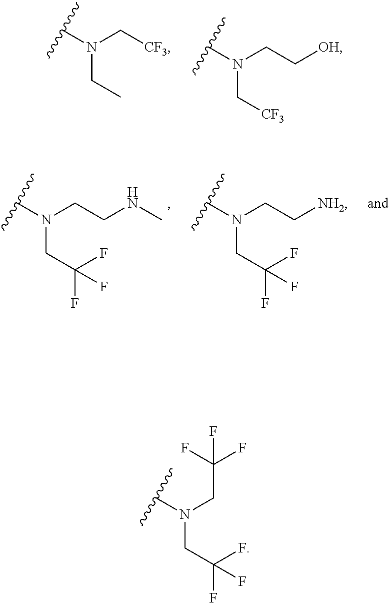

In some embodiments, for a compound or salt of Formula (I) or (I-a). R³ and R⁴ are each independently selected from (a) and (b):
(a) hydrogen;
(b) Ct alkyl optionally substituted with one or more substituents independently selected from:
   halogen, —OR¹¹, —SR¹¹, —N(R¹¹)₂, —C(O)R¹¹, —C(O)—O—C₁₋₆ alkyl, —OC(O)R¹¹, —OC(O)N(R¹¹)₂, —N(R¹¹)C(O)R¹⁵, —N(R¹⁵)C(O)OR¹⁵, —N(R¹¹)_{S})C(O)N(R¹¹)₂, —N(R¹¹)₅)S(O)₂(R¹¹) —S(O)R¹⁴, —S(O)₂R¹¹, —S(O)₂N(R⁵)₂, —NO₂, —CN;
   C₃₋₁₀ carbocycle and 3- to 10-membered heterocycle any of which is optionally substituted with one or more substituents independently selected from halogen, C₁₋₆ haloalkyl, C₁₋₆ alkoxy, —OR¹¹, —SR¹¹, —N(R¹¹)₂, —C(O)R¹¹, —C(O)OR¹¹, —OC(O)R¹⁴, —OC(O)N(R^{11B})₂, —C(O)N(R¹⁵)₂, —N(R^{1B})C(O)R¹¹, —N(R¹¹)C(O)OR¹¹, —N(R¹¹)C(O)N(R^{11B})₂, —N(R¹¹)S(O)(R^{11B}), —S(O)R¹¹, —S(O)₂R¹¹, —S(O)₂N(R¹¹)₂, —NO₂, and —CN.

In some embodiments, for a compound or salt of Formula (I) or (I-a), R³ and R⁴ are each independently selected from (a) and (b):
(a) hydrogen;
(b) C₁ alkyl optionally substituted with one or more substituents independently selected from:
   halogen, —OR¹¹, —SR¹¹, —N(R¹¹)₂, —C(O)R¹¹, —NO₂, —CN;
   C₃₋₁₀ carbocycle and 3- to 10-membered heterocycle any of which is optionally substituted with one or more substituents independently selected from halogen, C₁₋₆ haloalkyl, —OR¹¹, —SR¹¹, —N(R¹¹)₂, —C(O)R¹¹, —C(O)OR¹¹, —OC(O)R¹¹, —NO₂, and —CN.

In some embodiments, for a compound or salt of Formula (I) or (I-a), R³ and R⁴ are each independently selected from (a) and (b):
(a) hydrogen;
(b) C₁ alkyl optionally substituted with one or more substituents independently selected from:
   halogen, —OR¹¹, —SR¹¹, —N(R¹¹)₂, —C(O)R¹¹, —NO₂, —CN; C₃₋₆ carbocycle and 3- to 6-membered heterocycle any of which is optionally substituted with one or more substituents independently selected from halogen, C₁₋₆ haloalkyl, —OR¹¹, —SR¹¹, —N(R¹¹)₂, —C(O)R¹¹, —C(O)OR¹¹, —OC(O)R¹¹, —NO₂, and —CN.

In some embodiments, for a compound or salt of Formula (I) or (I-a), one of R³ and R⁴ is hydrogen, and the other of R³ and R⁴ is selected from C₁ alkyl optionally substituted with one or more substituents independently selected from;
halogen, —OR¹¹, —SR¹⁶, —N(R¹¹)₂, —C(O)R¹¹, —C(O)—O—C₁.alkyl, —OC(O)R¹¹, —OC(O)N(R¹⁵), —C(O)N(R¹¹)₂, —N(R¹¹)C(O)R¹¹, —N(R¹¹)C(O)OR¹¹, —N(R¹¹)C(O)N(R¹¹)₂, —N(R¹¹)S(O)₂(R¹⁴), —S(O)R¹², —S(O)₂R¹¹, —S(O)₂N(R¹¹)₂, —NO₂, and —CN;
C₃₋₁₀ carbocycle and 3- to 10-membered heterocycle any of which is optionally substituted with one or more substituents independently selected from halogen, C₁₋₆ haloalkyl, C₁₋₆ alkoxy, —OR¹¹, —SR¹¹, —N(R¹¹)₂, —C(O)R¹¹, —C(O)OR³, —OC(O)R¹¹, —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹⁴)₂, —N(R¹⁴)S(O)₂(R¹¹), —S(O)R¹¹, —S(O)₂R¹¹, —S(O)₂N(R¹¹)₂, —NO₂, and —CN.

In some embodiments, for a compound or salt of Formula (I) or (I-a), one of R³ and R⁴ is hydrogen, and the other of R³ and R⁴ is selected from CI alkyl optionally substituted with one or more substituents independently selected from:

halogen, —OR¹¹, —SR¹⁴, —N(R¹¹)₂, —C(O)R¹¹, —NO₂, —CN; C₃₋₆ carbocycle and 3- to 6-membered heterocycle any of which is optionally substituted with one or more substituents independently selected from halogen, C₁₋₆ haloalkyl, —OR¹², —SR¹⁵—
N(R¹¹)₂, —C(O)R¹¹, —C(O)OR¹¹, —OC(O)R¹¹, —NO₂, and —CN. In some embodiments, is

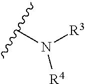

is represented by

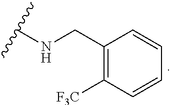 [SMILES: *NCc1ccccc1C(F)(F)F]

In some embodiments, one of R³ and R⁴ is hydrogen and

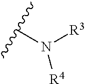

is represented by

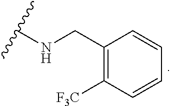 [SMILES: *NCc1ccccc1C(F)(F)F]

In some embodiments, for a compound or salt of Formula (I) or (I-a), R³ and R⁴ are each independently selected from (a) and (c):
(a) hydrogen; and
(c) C₂₋₆ alkyl optionally substituted with one or more substituents independently selected from:
   halogen, —OR¹¹, —SR¹¹, —N(R¹¹)₂, —C(O)R¹¹, —C(O)—O—CI—.alkyl, —OC(O)R¹², —OC(O)N(R¹¹)₂, —C(O)N(R¹¹)₂, —N(R¹¹)C(O)R¹⁵, —N(R¹¹)C(O)OR¹¹, —N(R¹¹)C(O)N(R¹¹)₂, —N(R¹¹)S(O)₂(R¹¹), —S(O)R¹¹, —S(O)₂R¹¹, —S(O)₂N(R¹¹)₂, —NO₂, and —CN;
   C₃₋₁₀ carbocycle and 3- to 10-membered heterocycle any of which is optionally substituted with one or more substituents independently selected from halogen, CI-6 haloalkyl, C₁₋₆ alkoxy, —OR¹¹, —SR¹¹, —N(R¹¹)₂, —C(O)R¹¹, —C(O)OR¹¹, —OC(O)R¹¹, —OC(O)N(R¹¹)₂, —C(O)N(R¹¹)₂, —N(R¹¹)C(O)R¹¹, —N(R¹¹)C(O)OR¹¹, —N(R⁵)C(O)N(R¹¹)₅)₂, —N(R¹⁵)S(O)₂(R¹⁵), —S(O)R¹¹, —S(O)₂R¹², —S(O)₂N(R¹¹)₂, —NO₂, and —CN.

In some embodiments, for a compound or salt of Formula (I) or (I-a), R³ and R⁴ are each independently selected from (a) and (c):
(a) hydrogen, and
(c) C₂₋₆ alkyl optionally substituted with one or more substituents independently selected from halogen, —OR¹¹, —SR¹¹, —N(R¹¹)₂, —C(O)R¹¹, —C(O)—O—C₁₋₆ alkyl, —OC(O)R¹¹, —OC(O)N(R¹¹)₂—C(O)N(R¹¹)₂, —N(R¹¹)C(O)R¹¹, —N(R¹¹)C(O)OR¹¹, —N(R¹¹)C(O)N(R¹¹)₂, —N(R¹¹)S(O)₂(R¹¹), —S(O)R¹⁵, —S(O)₂R′, —S(O)₂N(R¹⁵)₂, —NO₂, and —CN.

In some embodiments, for a compound or salt of Formula (I) or (I-a), R³ and R⁴ are each independently selected from (a) and (c):
(a) hydrogen; and
(c) C₂i alkyl optionally substituted with one or more substituents independently selected from halogen, —OR¹⁴, —N(R¹⁴)₂, —C(O)R¹¹, —C(O)N(R¹¹)₂, —N(R¹¹)C(O)R¹¹, —N(R¹⁴)C(O)OR¹¹, —NO₂, and —CN.

In some embodiments, for a compound or salt of Formula (I) or (I-a), one of R³ and R⁴ is hydrogen, and the other of R³ and R⁴ is C₂₋₆ alkyl optionally substituted with one or more substituents independently selected from halogen, —OR¹⁵, —N(R¹⁵)₂, —C(O)R¹⁵, —C(O)N(R⁵¹⁵)₂, ——N(R¹⁵)C (O) R¹⁵, —N(R¹⁵)C(O)OR¹⁵, —NO₂, and —CN. In some embodiments,

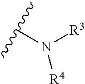

is represented by:

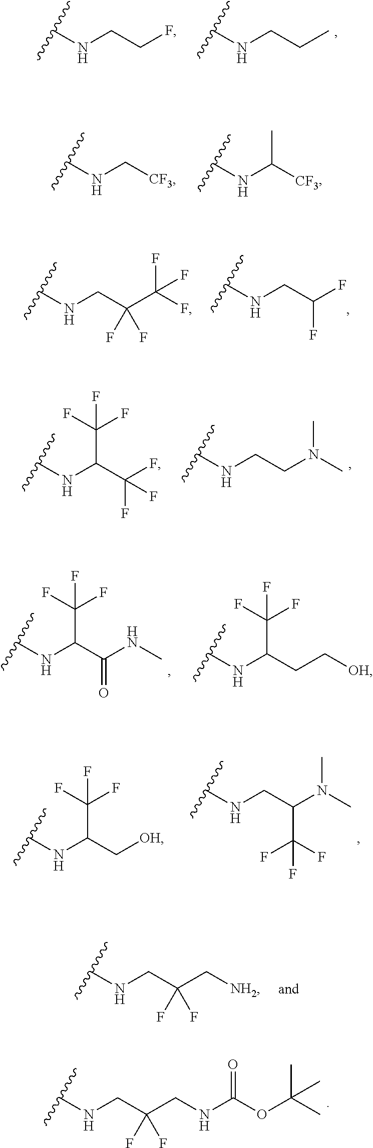

In some embodiments,

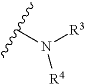

is represented by:

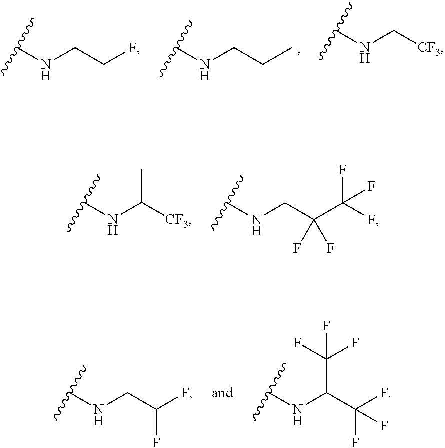

In some embodiments,

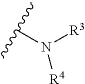

is represented by:

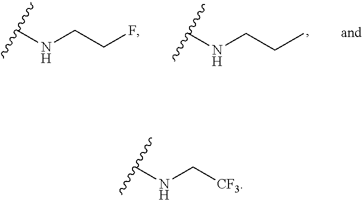

In some embodiments,

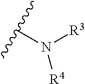

is represented by

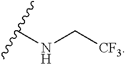 [SMILES: *NCC(F)(F)F]

In some embodiments, for a compound or salt of Formula (I) or (I-a), R³ and R⁴ is hydrogen, and

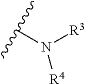

is represented by:

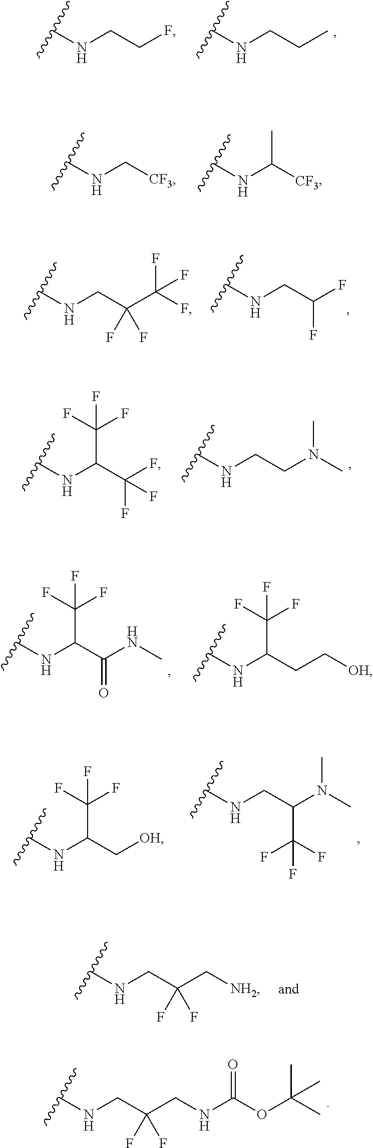

In some embodiments, one of R³ and R⁴ is hydrogen and

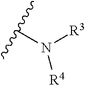

is represented by:

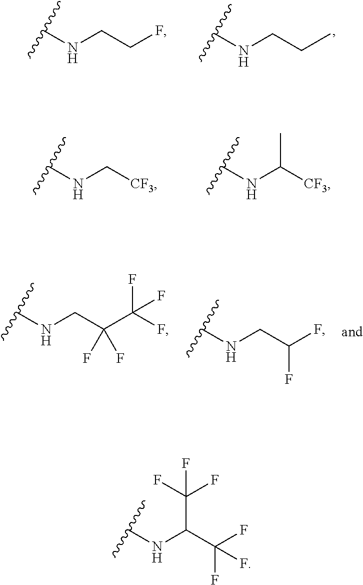

In some embodiments, one of R³ and R⁴ is hydrogen and

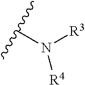

is represented by:

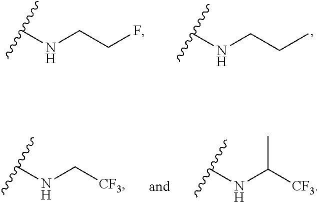

in some embodiments, one of R³ and R⁴ is hydrogen and

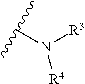

is represented by

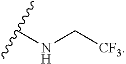 [SMILES: *NCC(F)(F)F]

In some embodiments, for a compound or salt of Formula (I) or (I-a), R³ and R⁴ are each independently selected from (a) and (c):
(a) hydrogen;
(c) C₂₋₆ alkyl optionally substituted with one or more substituents independently selected from: C₃₋₁₀ carbocycle and 3- to 10-membered heterocycle any of which is optionally substituted with one or more substituents independently selected from halogen, C₁₋₆ haloalkyl, C₁₋₆ alkoxy, -OR¹¹, —SR¹¹, —N(R¹⁵)₂, —C(O)R⁵, —C(O)OR¹¹, —OC(O)R¹¹, —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹¹)₂, —N(R⁵)S(O)₂(R¹¹), —S(O)R¹¹, —S(O)₂R¹¹, —S(O)₂N(R^{11B})₂, —NO₂, and —CN.

In some embodiments, for a compound or salt of Formula (I) or (I-a). R³ and R⁴ are each independently selected from (a) and (c):
(a) hydrogen;
(c) C₂₋₆ alkyl optionally substituted with one or more substituents independently selected from: C₃₋₆ carbocycle and 3- to 6-membered heterocycle each of which is optionally substituted with one or more substituents independently selected from halogen, Ci.(haloalkyl, Ci, alkoxy, —OR¹⁴, —SR¹⁴, —N(R¹⁵)₂, —C(O)R¹⁴, —C(O)OR¹⁴, —OC(O)R¹², —NO₂, and —CN.

In some embodiments, for a compound or salt of Formula (I) or (I-a), R³ and R⁴ are each independently selected from (a) and (c):
(a) hydrogen;
(c) C₂₋₆ alkyl optionally substituted with one or more substituents independently selected from: halogen; C₃₋₆ carbocycle and 3- to 6-membered heterocycle each of which is optionally substituted with one or more substituents independently selected from halogen. C₁₋₆haloalkyl, —OR¹¹, —N(R¹¹)₂, —C(O)R¹¹, —NO₂, and —CN. In some embodiments,

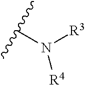

is represented by:

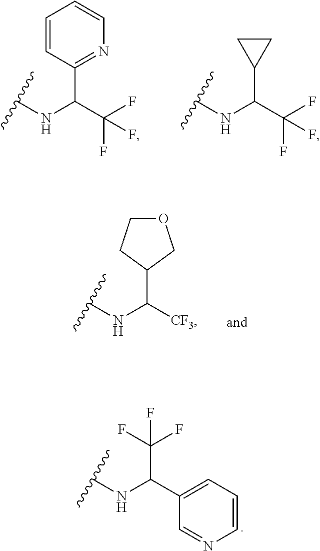 [SMILES: *NC(c1ccccn1)C(F)(F)F ; *NC(C1CC1)C(F)(F)F ; *NC(C1CCOC1)C(F)(F)F ; *NC(c1cccnc1)C(F)(F)F]

In some embodiments, R¹ is

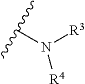

represented by:

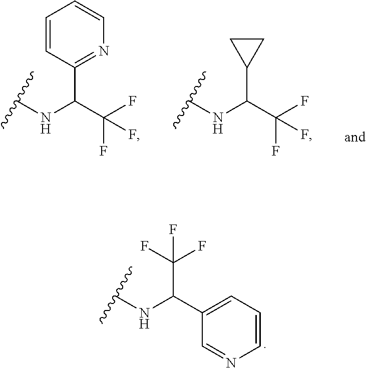 [SMILES: *NC(c1ccccn1)C(F)(F)F ; *NC(C1CC1)C(F)(F)F ; *NC(c1cccnc1)C(F)(F)F]

In some embodiments, for a compound or salt of Formula (I) or (I-a), one of R³ and R⁴ is hydrogen and the other of R³ and R⁴ is C₂₋₆ alkyl optionally substituted with one or more substituents independently selected from: halogen: C₃₋₆ carbocycle and 3- to 6-membered heterocycle each of which is optionally substituted with one or more substituents independently selected from halogen, C₁₋₆ haloalkyl, —OR¹¹, —N(R¹¹)₂, —C(O)R¹¹, —NO₂, and —CN. In some embodiments, one of R³ and R⁴ is hydrogen, and

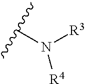

s represented by:

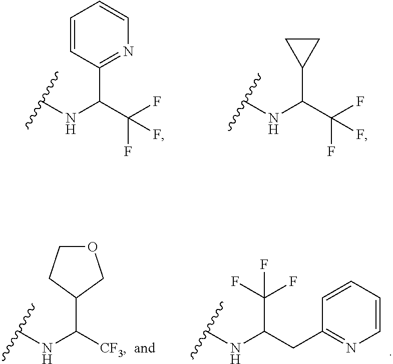

In some embodiments, one of R³ and R⁴ is hydrogen, and

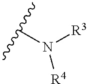

is represented by:

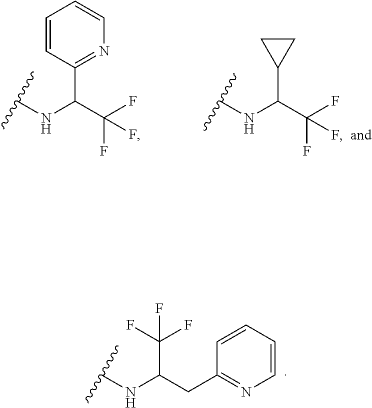

In some embodiments, for a compound or salt of Formula (I) or (I-a), R³ and R⁴ are each independently selected from (a) and (d):
(a) hydrogen; and
(d) C₃₋₁₀ carbocycle and 3- to 10-membered heterocycle any of which is optionally substituted with one or more substituents independently selected from:
   halogen, —OR¹¹, —SR¹¹, —N(R¹¹)₂, —C(O)R¹¹, —C(O)OR¹¹, —OC(O)R¹¹, —OC(O)N(R¹¹)₂. —C(O)N(R¹⁴)₂, —N(R¹⁴)C(O)R¹¹, —N(R¹¹)C(O)OR¹¹, —N(R¹¹)C(O)N(R^{1B})₂, —N(R¹¹)S(O)₂(R¹¹), —S(O)R¹¹, —S(O)₂R¹¹, —S(O)₂N(R¹¹)₂, —NO₂, and —CN; and
   C₁₋₆ alkyl optionally substituted with one or more substituents independently selected from halogen, —OR¹¹, —SR¹¹, —N(R¹¹)₂, —C(O)R¹¹, —C(O)OR¹², —OC(O)R¹⁴, —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹¹)₂, —N(R^{11B})S(O)₂(R¹¹), —S(O)R¹¹, —S(O)₂R¹⁵, —S(O)₂N(R¹⁵)₂, —NO₂, and —CN.

In some embodiments, for a compound or salt of Formula (I) or (I-a), R³ and R⁴ are each independently selected from (a) and (d):
(a) hydrogen; and
(d) C₃₋₁₀ carbocycle and 3- to 10-membered heterocycle any of which is optionally substituted with one or more substituents independently selected from: halogen, —OR¹¹, —SR¹², —N(R¹⁴)₂, —C(O)R¹¹, —C(O)OR¹¹, —OC(O)R¹¹, —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹¹)₂, —N(R¹¹)S(O)₂(R¹¹), —S(O)R¹¹, —S(O)₂R¹¹, —S(O)₂N(R¹¹)₂, —NO₂, and —CN.

In some embodiments, for a compound or salt of Formula (I) or (I-a), R³ and R⁴ are each independently selected from (a) and (d):
(a) hydrogen; and
(d) C₃₋₆ carbocycle and 3- to 6-membered heterocycle any of which is optionally substituted with one or more substituents independently selected from: halogen, —OR¹¹, —SR¹¹, —N(R¹¹)₂, —C(O)R¹², —C(O)OR¹¹, —OC(O)R¹¹, —NO₂, and —CN.

In some embodiments, for a compound or salt of Formula (I) or (I-a). R³ and R⁴ are each independently selected from (a) and (d):
(a) hydrogen; and
(d) C₃₋₁₀ carbocycle and 3- to 10-membered heterocycle any of which is optionally substituted with one or more substituents independently selected from: C₁₋₆ alkyl optionally substituted with one or more substituents independently selected from halogen, —OR¹¹, —SR¹¹, —N(R¹¹)₂, —C(O)R¹¹)₅, —C(O)OR¹¹, —OC(O)R¹¹, —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹¹)₂, —N(R¹¹)S(O)₂(Rts), —S(O)R¹¹, —S(O)₂R¹¹, —S(O)₂N(R¹¹)₂, —NO₂, and —CN.

In some embodiments, for a compound or salt of Formula (I) or (I-a), R³ and R⁴ are each independently selected from (a) and (d):
(a) hydrogen; and
(d) C₃₋₆ carbocycle and 3- to 6-membered heterocycle any of which is optionally substituted with C₁₋₃ alkyl optionally substituted with one or more substituents independently selected from halogen, —OR¹¹, —SR¹¹, —N(R¹¹)₂, —C(O)R¹¹, —C(O)OR¹³, —OC(O)R¹¹, —NO₂, and —CN.

In some embodiments, for a compound or salt of Formula (I) or (I-a),

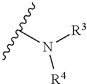

is represented by:

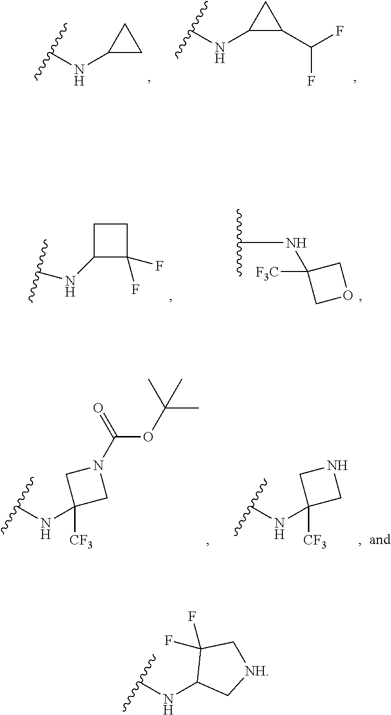

In some embodiments,

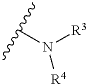

is represented by:

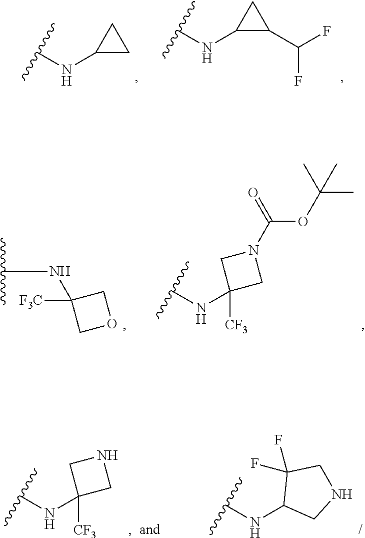 [SMILES: *NC1CC1 ; *NC1CC1C(F)F ; *NC1(C(F)(F)F)COC1 ; *NC1(C(F)(F)F)CN(C(=O)OC(C)(C)C)C1 ; *NC1(C(F)(F)F)CNC1 ; *NC1CNCC1(F)F]

In some embodiments,

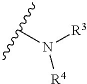

is represented by:

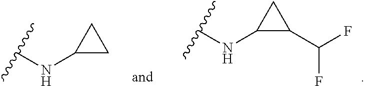 [SMILES: *NC1CC1 ; *NC1CC1C(F)F]

in some embodiments,

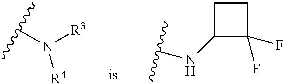

In some embodiments,

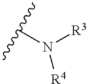

is represented by:

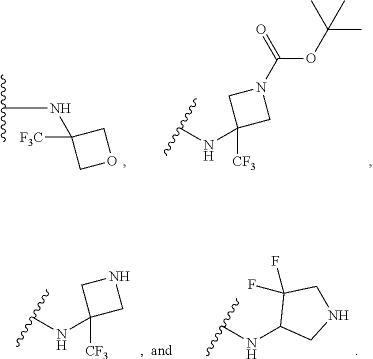 [SMILES: *NC1(C(F)(F)F)COC1 ; *NC1(C(F)(F)F)CN(C(=O)OC(C)(C)C)C1 ; *NC1(C(F)(F)F)CNC1 ; *NC1CNCC1(F)F]

In some embodiments, for a compound or salt of Formula (I) or (I-a), one of R³ and R⁴ is

hydrogen, and R¹⁴

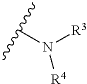

is represented by:

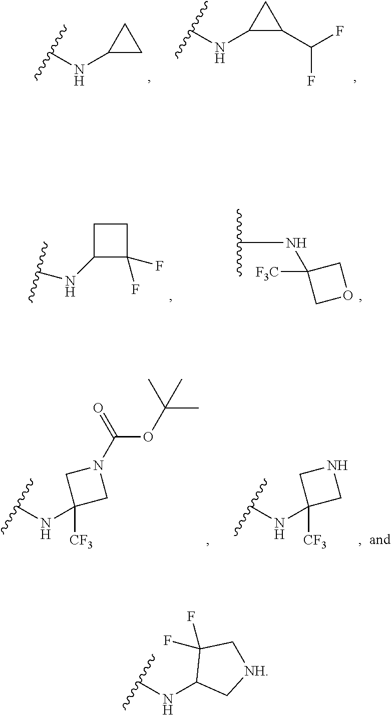

In some embodiments, one of R³ and R⁴ is hydrogen, and

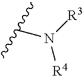

is represented by:

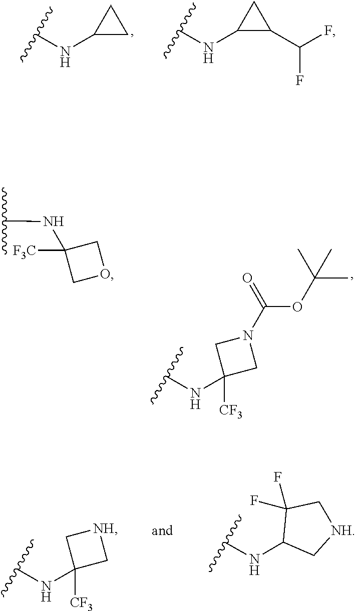 [SMILES: *NC1CC1 ; *NC1CC1C(F)F ; *NC1(C(F)(F)F)COC1 ; *NC1(C(F)(F)F)CN(C(=O)OC(C)(C)C)C1 ; *NC1(C(F)(F)F)CNC1 ; *NC1CNCC1(F)F]

In some embodiments, one of R³ and R⁴ is hydrogen, and

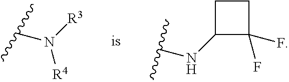

some embodiments, one of R³ and R⁴ is hydrogen, and

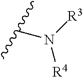

is represented by:

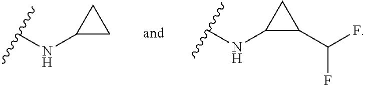 [SMILES: *NC1CC1 ; *NC1CC1C(F)F]

In some embodiments, one of R³ and R⁴ is hydrogen, and

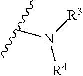

and is represented by

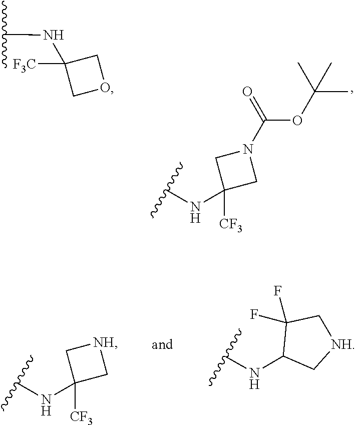 [SMILES: *NC1(C(F)(F)F)COC1 ; *NC1(C(F)(F)F)CN(C(=O)OC(C)(C)C)C1 ; *NC1(C(F)(F)F)CNC1 ; *NC1CNCC1(F)F]

In some embodiments, for a compound or salt of Formula (I) or (I-a), R³ and R⁴ are each independently selected from (b) and (c):
(b) C, alkyl optionally substituted with one or more substituents independently selected from; halogen, —OR¹¹, —SR¹¹, —N(R¹¹)₂, —C(O)R¹¹, —C(O)—O—C₁₋₆ alkyl, —OC(O)R¹¹, —OC(O)N(R¹¹)₂, —N(R¹¹)C(O)R¹⁵, —N(R¹⁵)C(O)OR¹⁵, —N(R¹¹)_{S})C(O)N(R¹¹)₂, —N(R¹¹)₅)S(O)₂(R¹¹), —S(O)R¹¹)_{S}, —S(O)₂R¹¹, —S(O)₂N(R⁵)₂, —NO₂, —CN;
C₃₋₁₀ carbocycle and 3- to 10-membered heterocycle any of which is optionally substituted with one or more substituents independently selected from halogen, C₁₋₆ haloalkyl, C₁₋₆ alkoxy, —OR¹¹, —SR¹¹, —N(R¹¹), —C(O)R¹⁴, —C(O)OR¹¹, —OC(O)R¹⁴, —OC(O)N(R¹¹)₂, —C(O)N(R¹⁵)₂, —N(R¹⁴)C(O)R¹⁵, —N(R¹⁵)C(O)OR¹⁵, —N(R¹¹)₅)C(O)N(R¹¹)₅)₂, —N(R¹¹)S(Oh(R¹¹), —S(O)R¹¹, —S(O)₂R¹¹, —S(O)₂N(R¹¹)₂, —NO₂, and —CN; (c) C₂₋₆ alkyl optionally substituted with one or more substituents independently selected from:
halogen, —OR¹¹)_{S}, —SR¹¹, —N(R¹⁵)₂, —C(O)R¹¹, —C(O)—O—C-₆ alkyl, —OC(O)R¹¹, —OC(O)N(R¹¹)₂, —C(O)N(R¹¹)₂, —N(R¹¹)C(O)R¹¹, —N(R¹⁴)C(O)OR¹¹, —N(R¹¹)C(O)N(R¹¹)₂, —N(R¹⁵)S(O)₂(R¹⁵), —S(O)R¹¹, —S(O)₂R¹¹, —S(O)₂N(R¹¹)₂, —NO₂, and —CN;
C₃₋₁₀ carbocycle and 3- to 10-membered heterocycle any of which is optionally substituted with one or more substituents independently selected from halogen. C₁₋₆ haloalkyl, C₁₋₆ alkoxy, —OR¹¹, —SR¹¹, —N(R¹¹)₂, —C(O)R¹¹, —C(O)OR¹¹, —OC(O)R¹⁵, —OC(O)N(R¹¹)₂, —C(O)N(R¹¹)₂, —N(R¹¹)C(O)R¹⁵, —N(R¹¹)₃)C(O)OR¹², —N(R¹⁴)C(O)N(R¹⁴)₂, —N(R¹⁵)S(O)₂(R¹⁴), —S(O)R¹¹, —S(O)₂R¹¹, —S(O)₂N(R¹¹)₂, —NO₂, and —CN.

In some embodiments, for a compound or salt of Formula (I) or (I-a), R³ and R⁴ are each independently selected from (b) and (c):
(b) C, alkyl optionally substituted with one or more substituents independently selected from; halogen, —OR¹¹, —SR¹¹, —N(R¹¹)₂, —C(O)R¹¹, —OC(O)R¹¹, —OC(O)N(R¹¹)₂, —NO₂, —CN; C₃₋₆ carbocycle and 3- to 6-membered heterocycle any of which is optionally substituted with one or more substituents independently selected from halogen, C₁₋₆ haloalkyl, —OR¹¹, —SR¹¹, —N(R¹¹)₂, —C(O)R¹¹, —C(O)OR¹¹, —OC(O)R¹¹, —NO₂, and —CN.
(c) C₂₋₆ alkyl optionally substituted with one or more substituents independently selected from:
   halogen, —OR¹¹, —SR¹¹, —N(R¹¹)₂, —C(O)R¹⁵, —OC(O)R¹¹, —OC(O)N(R¹¹)₂, —NO₂, —CN;
   C₃₋₆ carbocycle and 3- to 6-membered heterocycle any of which is optionally substituted with one or more substituents independently selected from halogen, C₁₋₆ haloalkyl, —OR¹¹, —SR¹¹, —N(R¹⁵)₂, —C(O)R¹⁵, —C(O)OR¹³, —OC(O)R¹¹, —NO₂, and —CN.

In some embodiments, for a compound or salt of Formula (I) or (I-a),

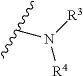

is represented by

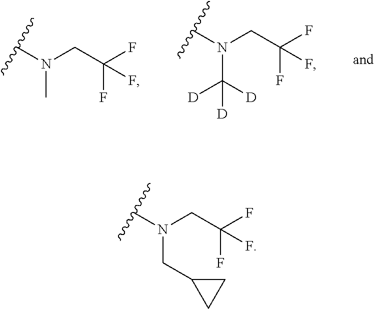

In some embodiments, one of R³ and R¹⁴ is methyl, and

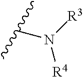

is represented by

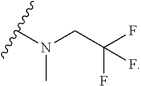 [SMILES: *N(C)CC(F)(F)F]

In some embodiments, one of R³ and R⁴is optionally substituted methyl, and

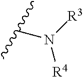

represented by

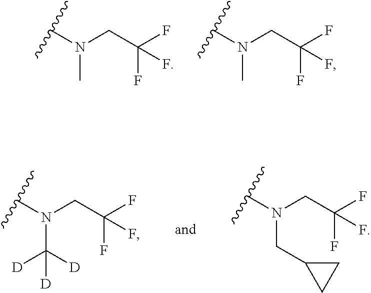

In some embodiments,

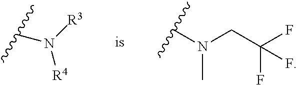

In some embodiments, for a compound or salt of Formula (I) or (I-a), R³ and R⁴ are each independently selected from (b) and (d):
(b) C₁ alkyl optionally substituted with one or more substituents independently selected from; halogen, —OR¹¹, —SR¹², —N(R⁵)₂, —C(O)R¹⁴, —C(O)—O—C₁₋₆-alkyl, —OC(O)R¹⁴, —OC(O)N(R¹¹)₂, —N(R¹¹)C(O)R¹¹)₅, —N(R¹³)C(O)OR¹¹)₅, —N(R¹¹)₅)C(O)N(R¹¹)₅)₂, —N(R¹¹)S(O)₂(R¹¹)₅), —S(O)R^{11B}, —S(O)₂R¹¹)₅,—S(O)₂N(R¹⁵)₂, —NO₂, —CN;
C₃₋₁₀ carbocycle and 3- to 10-membered heterocycle any of which is optionally substituted with one or more substituents independently selected from halogen, C₁₋₆ haloalkyl, C₁_- alkoxy, —OR¹¹, —SR¹¹, —N(R¹¹)₂, —C(O)R¹¹)_{S}, —C(O)OR¹³, —OC(O)R¹¹)_{S}, —OC(O)N(R¹⁴)₂, —C(O)N(R¹¹)₂, —N(R^{1′})C(O)R¹², —N(R¹¹)C(O)OR¹², —N(R)C(O)N(R¹⁵)₂, —N(R¹¹)₃)S(O)₂(R¹¹), —S(O)R¹¹, —S(O)₂R^{1S}, —S(O)₂N(R¹¹)₂, —NO₂, and —CN; and
(d) C₃₋₁₀ carbocycle and 3- to 10-membered heterocycle any of which is optionally substituted with one or more substituents independently selected from:
   halogen, —OR^{11B}, —SR¹⁵, —N(R¹¹)₂, —C(O)R¹¹, —C(O)OR¹¹, —OC(O)R^{11B}, —OC(O)N(R¹¹)₂, —C(O)N(R¹¹)₂, —N(R¹⁵)C(O)R¹⁵, —N(R¹³)C.(O)OR¹⁵, —N(R¹¹)C(O)N(R¹¹)₂, —N(R¹⁵)S(O)₂(R¹⁵), —S(O)R¹¹, —S(O)₂R¹¹, —S(O)₂N(R¹⁵)₂, —NO₂, and —CN; and
   C₁₋₆ alkyl optionally substituted with one or more substituents independently selected from halogen, —OR¹⁵, —SR¹⁵, —N(R¹¹)₂, —C(O)R¹¹, —C(O)OR¹¹, —OC(O)R¹¹, —OC(O)N(R⁵)₂, —C(O)N(R¹⁵)₂, —N(R^{1S})C(O)R¹⁵, —N(R¹⁵)C(O)OR¹⁵, —N(R¹⁵)C(O)N(R⁵)₂, —N(R^{11B})S(O)₂(R¹³), —S(O)R¹¹, —S(O)₂R¹¹, —S(O)₂N(R¹¹)₅)₂, —NO₂, and —CN.

In some embodiments, for a compound or salt of Formula (I) or (I-a), R³ and R⁴ are each independently selected from (b) and (d):
(b) C₁ alkyl optionally substituted with one or more substituents independently selected from:
   halogen, —OR¹⁵, —SR¹⁵, —N(R¹⁵)₂, —C(O)R¹¹, —OC(O)R¹³, —NO₂, —CN;
   C₃₋₆ carbocycle and 3- to 6-membered heterocycle any of which is optionally substituted with one or more substituents independently selected from halogen, C₁₋₆ haloalkyl, C₁₋₆ alkoxy, —OR¹⁵, —SR¹¹, —N(R¹⁴)₂, —C(O)R¹¹, —C(O)OR¹¹, —OC(O)R¹¹, —NO₂, and —CN; and
(d) C₃₋₆ carbocycle and 3- to 6-membered heterocycle any of which is optionally substituted with one or more substituents independently selected from:
   halogen, —OR¹¹, —SR¹¹, —N(R¹¹)₅)₂, —C(O)R¹¹, —OC(O)R¹¹, —NO₂, —CN; and
   C₁₋₆ alkyl optionally substituted with one or more substituents independently selected from halogen, C₁₋₆ haloalkyl, C₁₋₆ alkoxy, —OR¹¹, —SR¹¹, —N(R¹¹)₂, —C(O)R¹⁴, —C(O)OR¹³, —OC(O)R¹¹, —NO₂, and —CN.

In some embodiments, for a compound or salt of Formula (I) or (I-a),

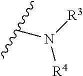

is represented by

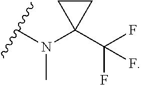 [SMILES: *N(C)C1(C(F)(F)F)CC1]

In some embodiments, one of R³ and R⁴ is methyl, and

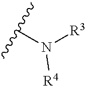

is represented by

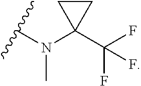 [SMILES: *N(C)C1(C(F)(F)F)CC1]

In some embodiments, for a compound or salt of Formula (I) or (I-a), R³ and R⁴ are each independently selected from (a), (b), and (c):
(a) hydrogen;
(b) C, alkyl optionally substituted with one or more substituents independently selected from:
   halogen, —OR¹⁵, —SR¹¹, —N(R¹¹)₂, —C(O)R¹¹, —C(O)—O—C₁₋₆.alkyl, —OC(O)R¹¹, —OC(O)N(R¹¹)₂, —N(R¹¹)C(O)R¹¹, —N(R¹¹)C(O)OR¹⁴, —N(R¹⁴)C(O)N(R¹¹)₂, —N(R¹¹)S(O)₂(R¹¹), —S(O)R¹¹, —S(O)₂R¹¹, —S(O)₂N(R¹¹)₂, —NO₂, —CN;
   C₃₋₁₀ carbocycle and 3- to 10-membered heterocycle any of which is optionally substituted with one or more substituents independently selected from halogen, C₁₋₆haloalkyl, C₁₋₆ alkoxy, —OR¹⁵, —SR⁵, —N(R¹¹)₂, —C(O)R¹¹, —C(O)OR¹³, —OC(O)R^{1S}, —OC(O)N(R¹¹)₂—C(O)N(R¹¹)₂, —N(R¹¹)C(O)R¹¹, —N(R¹⁵)C(O)OR¹⁵, —N(R¹¹)C(O)N(R¹¹)₂, —N(R¹¹)₅)S(O)₂(R¹¹), —S(O)R¹¹, —S(O)₂R¹⁵, —S(O)₂N(R¹¹)₂, —NO₂, and —CN; and
(c) C₂₋₆ alkyl optionally substituted with one or more substituents independently selected from:
   halogen, —OR¹¹, —SR¹¹, —N(R¹¹)₂, —C(O)R¹¹)_{S}, —C(O)—O—C₁—.-alkyl, —OC(O)R¹¹, —OC(O)N(R¹¹)₂, —C(O)N(R¹¹)₂, —N(R¹¹)C(O)R¹⁴, —N(R¹⁴)C(O)OR¹², —N(R¹¹)C(O)N(R¹¹)₂, —N(R¹¹)S(O)₂(R¹¹), —S(O)R¹⁵, —S(O)₂R¹², —S(O)₂N(R¹¹)₂, —NO₂, and —CN; and
   C₃₋₁₀ carbocycle and 3- to 10-membered heterocycle any of which is optionally substituted with one or more substituents independently selected from halogen, C₁₋₆ haloalkyl, C₁₋₄ alkoxy, —OR¹¹, —SR¹¹, —N(R¹¹)₂, —C(O)R¹¹, —C(O)OR¹¹, —OC(O)R¹¹, —OC(O)N(R¹¹)₂, —C(O)N(R¹⁴)₂, —N(R¹⁴)C(O)R¹⁴—N(R¹¹)C(O)OR, —N(R¹¹)C(O)N(R¹¹)₂, —N(R¹¹)S(O)₂(R¹¹), —S(O)R¹⁴, —S(O)₂R¹⁵, —S(O)₂N(R¹¹)₂, —NO₂, and —CN.

In some embodiments, for a compound or salt of Formula (I) or (I-a), R³ and R¹ are each independently selected from (a), (b), and (c):
(a) hydrogen;
(b) C₁ alkyl optionally substituted with one or more substituents independently selected from:
   halogen, —OR¹¹, —SR¹¹, —N(R¹¹)₂, —C(O)R¹¹, —OC(O)R¹¹, —NO₂, —CN;
   C₃₋₆ carbocycle and 3- to 6-membered heterocycle any of which is optionally substituted with one or more substituents independently selected from halogen, C₁₋₆ haloalkyl, —OR¹⁵, —SR¹¹, —N(R¹⁵)₂, —C(O)R¹¹, —C(O)OR¹¹, —OC(O)R¹⁴, —NO₂, and —CN; and
(c) C₂₋₆ alkyl optionally substituted with one or more substituents independently selected from:
   halogen, —OR¹¹, —SR¹¹, —N(R¹¹)₂, —C(O)R¹¹, —OC(O)R¹¹, —NO₂, and —CN; and
   C₃₋₆ carbocycle and 3- to 6-membered heterocycle any of which is optionally substituted with one or more substituents independently selected from halogen, haloalkyl, —OR¹⁵, —SR¹¹, —N(R¹⁵)₂, —C(O)R¹¹, —C(O)OR¹¹, —OC(O)R¹¹)₅, —NO₂, and —CN.

In some embodiments, for a compound or salt of Formula (I) or (I-a), R³ and R⁴ are each independently selected from (a), (b), (c), and (d):
(a) hydrogen;
(b) C₁ alkyl optionally substituted with one or more substituents independently selected from halogen, —OR¹⁴, —N(R¹¹)₂, —C(O)R¹¹, —NO₂, —CN; and C₃—carbocycle optionally substituted with one or more substituents independently selected from halogen. C₁₋₆haloalkyl, —OR¹¹, —N(R¹¹)₂, —C(O)R¹¹, —NO₂, and —CN;
(c) C₂, alkyl optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —N(R¹¹)C(O)OR¹¹, —NO₂, —CN; C₃₋₆ carbocycle and 3- to 6-membered heterocycle any of which is optionally substituted with one or more substituents independently selected from halogen, —OR¹⁵, —SR¹⁵, —N(R¹⁴)₂, —C(O)R¹⁴, —NO₂, and —CN; and
(d) C₃₋₆ carbocycle and 3- to 6-membered heterocycle each of which is optionally substituted with one or more substituents independently selected from halogen, —OR¹¹, —SR¹¹, —N(R¹¹)₂, —C(O)R¹¹, —NO₂, —CN; and C₁₋₆ alkyl optionally substituted with one or more substituents independently selected from halogen, —OR¹¹, —SR¹¹, —N(R¹¹)₂, —C(O)R¹¹, —NO₂, and —CN.

In some embodiments, for a compound or salt of Formula (I) or (I-a), R³ or R¹⁴ is selected from Ct alkyl optionally substituted with one or more substituents independently selected from halogen, —C(O)N(R¹¹)₂, —OR¹⁵; and cyclopropyl, cyclobutyl, cyclopentyl, cyclohexyl, and phenyl, each of which is optionally substituted with one or more substituents independently selected from halogen, Ct-₃ haloalkyl and —OR¹⁴.

In some embodiments, for a compound or salt of Formula (I) or (I-a), R³ or R¹⁴ is selected from methyl,

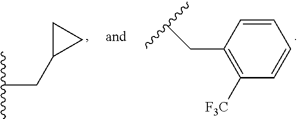 [SMILES: *CC1CC1 ; *Cc1ccccc1C(F)(F)F]

In some embodiments, for a compound or salt of Formula (I) or (I-a), R³ or R¹⁴ is selected from C₂₋₄ alkyl optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)OR¹¹, —NO₂, —CN; C₃₋₆ carbocycle and 3- to 6-membered heterocycle any of which is optionally substituted with one or more substituents independently selected from halogen, —OR¹¹, —SR¹¹, —N(R¹¹)₂, —C(O)R¹¹, —NO₂, and —CN.

In some embodiments, for a compound or salt of Formula (I) or (I-a), R³ or R¹⁴ is selected from ethyl, propyl, isopropyl, isobutyl, and butyl, each of which is optionally substituted with one or more substituents independently selected from chloro, fluoro, bromo, —OR¹⁴, —N(R⁵)₂, —C(O)N(R¹¹)₂ and —N(R¹¹)C(O)OR.

In some embodiments, for a compound or salt of Formula (I) or (I-a), R³ or R¹⁴ is selected from

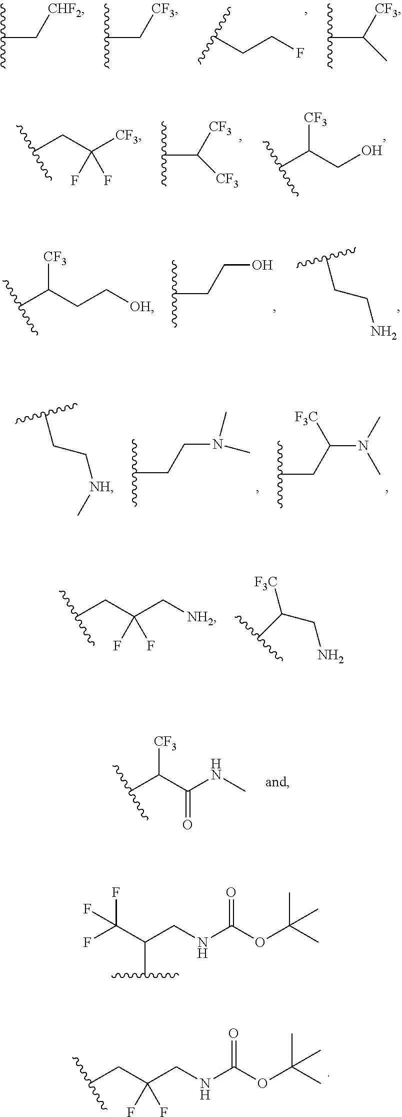

In some embodiments, for a compound or salt of Formula (I) or (I-a), R³ or R⁴ is selected from

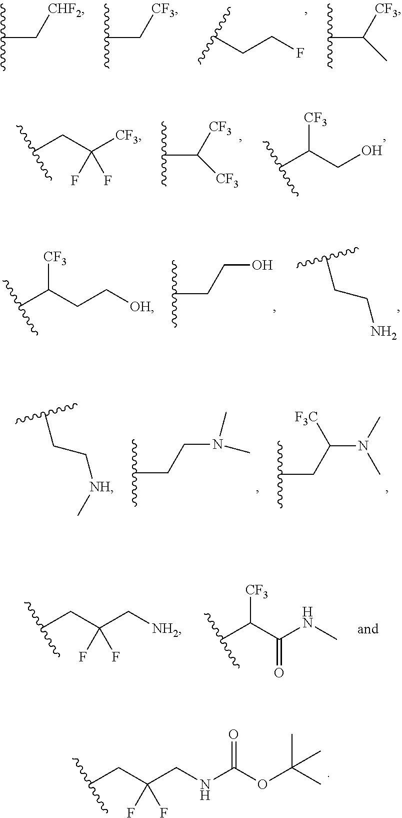

In some embodiments, for a compound or salt of Formula (I) or (I-a), R³ or R¹ is selected from C₂₋₆ alkyl optionally substituted with one or more substituents independently selected from halogen, —OR¹¹, —N(R¹¹)₂, C₃₋₆ carbocycle and 3- to 6-membered heterocycle any of which is optionally substituted with one or more substituents independently selected from halogen, —OR¹¹)₅, —SR¹¹, —N(R¹¹)₂, —C(O)R¹⁴, —NO₂, and —CN.

In some embodiments, for a compound or salt of Formula (I) or (I-a), R³ or R¹⁴ is selected from

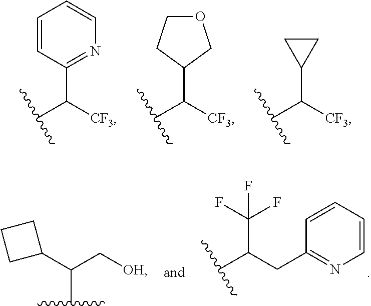 [SMILES: *C(c1ccccn1)C(F)(F)F ; *C(C1CCOC1)C(F)(F)F ; *C(C1CC1)C(F)(F)F ; *C(CO)C1CCC1 ; *C(Cc1ccccn1)C(F)(F)F]

In some embodiments, R³ or R⁴ is selected from

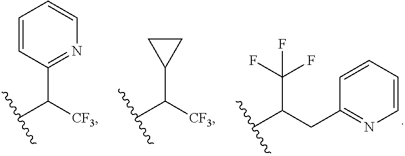 [SMILES: *C(c1ccccn1)C(F)(F)F ; *C(C1CC1)C(F)(F)F ; *C(Cc1ccccn1)C(F)(F)F]

In some embodiments, for a compound or salt of Formula (I) or (I-a), R³ or R¹⁴ is selected from cyclopropyl, cyclobutyl, cyclopentyl, cyclohexyl, phenyl, oxetanyl, azetidine, pyrrolidinyl, and pyridinyl, each of which is optionally substituted with one or more substituents independently selected from chloro, fluoro, bromo, —OR¹⁵, —NO₂, —CN; and C₁₋₃ alkyl optionally substituted with one or more substituents independently selected from chloro, fluoro, bromo, —OR¹⁴, —NO₂, and —CN.

In some embodiments, for a compound or salt of Formula (I) or (I-a), R³ or R¹⁴ is selected

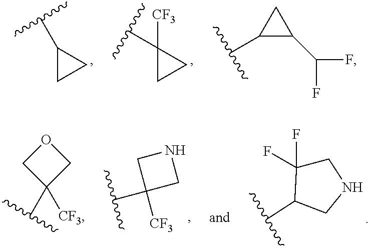

In some embodiments, for a compound or salt of Formula (I) or (I-a), R³ and R⁴ of

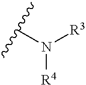

come together to form a 4- to 12-membered heterocycle any one of which is optionally substituted with one or more substituents independently selected from:
halogen, —OR¹⁶, —SR¹⁶, —N(R¹⁶)₂, —C(O)R¹⁶, —C(O)OR⁶, —OC(O)R¹⁶, —OC(O)N(R¹⁶)₂, —C(O)N(R¹⁶)₂, —N(R¹⁶)C(O)R¹⁶, —N(R¹⁶)C(O)OR¹⁶, —N(R¹⁶)C(O)N(R¹⁶)₂, —NO₂, ═O, and —CN; and
C₁₋₆ alkyl optionally substituted with one or more substituents independently selected from halogen, —OR⁶, —SR⁶, —N(R¹⁶)₂, —C(O)R¹⁶, —NO₂, and —CN.

In some embodiments, for a compound or salt of Formula (I) or (I-a), R³ and R⁴ of

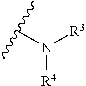

come together to form an optionally substituted 4- to 12-membered heterocycle comprising more than one heteroatom selected from oxygen, nitrogen, sulfur, and any combination thereof. In some embodiments, the optionally substituted 4- to 12-membered heterocycle optionally comprises more than one heteroatom selected from oxygen, nitrogen, and any combination thereof. In some embodiments, the optionally substituted 4- to 12-membered heterocycle optionally comprises more than one heteroatom selected from oxygen, sulfur, and any combination thereof. In some embodiments, the optionally substituted 4- to 12-membered heterocycle optionally comprises more than one heteroatom selected from nitrogen and sulfur and any combination thereof. In some embodiments, the optionally substituted 4- to 12-membered heterocycle optionally comprises more than one heteroatom selected from oxygen and nitrogen. In some embodiments, the optionally substituted 4- to 12-membered heterocycle optionally comprises more than one heteroatom selected from oxygen and sulfur. In some embodiments, the optionally substituted 4- to 12-membered heterocycle comprises more than one heteroatom selected from nitrogen and sulfur. In some embodiments, the optionally substituted 4- to 12-membered heterocycle optionally comprises at least one additional heteroatom selected from oxygen, nitrogen, sulfur, and any combination thereof. In some embodiments, the optionally substituted 4- to 12-membered heterocycle comprises one nitrogen heteroatom. In some embodiments, for a compound or salt of Formula (I) or (I-a), the 4- to 12-membered heterocycle is selected from a saturated 4- to 8-membered heterocycle and 6- to 12-membered unsaturated heterocycle, any one of one which is optionally substituted. In some embodiments, the R³ and R⁴ of

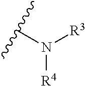

come together to form a saturated 4- to 8-membered heterocycle any one of which is optionally substituted.

In some embodiments, for a compound or salt of Formula (I) or (I-a), R³ and R⁴ come together to form a saturated 4- to 8-membered heterocycle and the saturated 4- to 8-membered heterocycle is selected from azetidine, oxetane, pyrrolidine, pyrazolidine, imidazolidine, oxazolidine, tetrahydrofuran, tetrahydrothiophene, piperidine, piperazine, tetrahydropyran, morpholine, oxazepane, and azaspirol[3.3]heptane, any one of which is optionally substituted with one substituents independently selected from chloro, fluoro, —OR¹⁶, —N(R¹⁶)₂, —C(O)OR¹⁶, —C(O)N(R⁶)₂, —N(R¹⁶)C(O)R¹⁶, —NO₂, and —CN; and C₁₋₃ alkyl optionally substituted with one or more substituents independently selected from chloro, fluoro, OR¹⁶, —NO₂, and —CN.

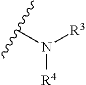

In some embodiments, for a compound or salt of Formula (I) or (I-a), R³ and R⁴ of come together to form the saturated 4- to 8-membered heterocycle and the 4- to 8-membered

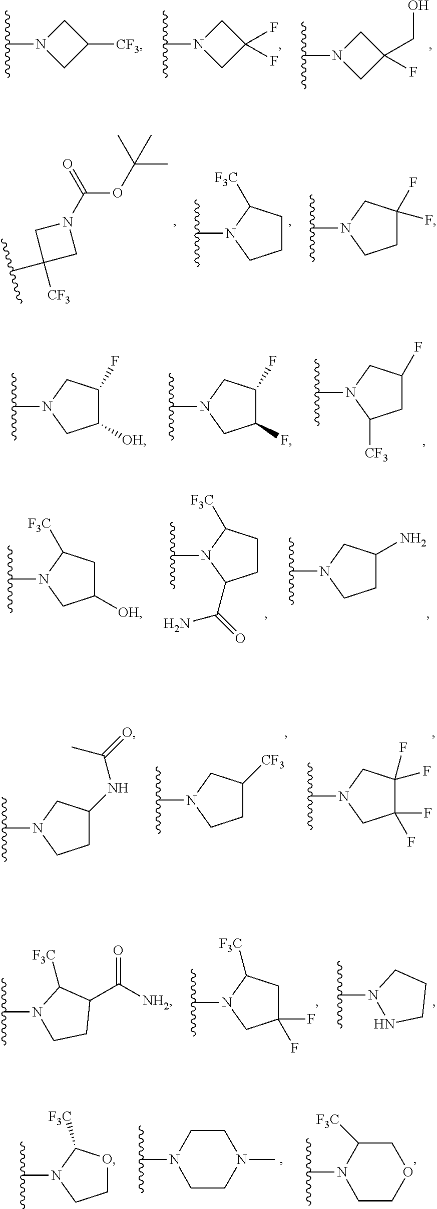

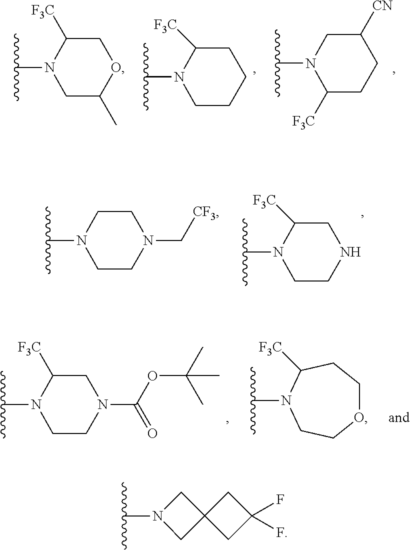

In some embodiments, for a compound or salt of Formula (I) or (I-a), R³ and R⁴ of

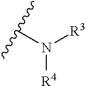

come together to form the saturated 4- to 8-membered heterocycle and the 4- to 8-membered heterocycle is selected from:

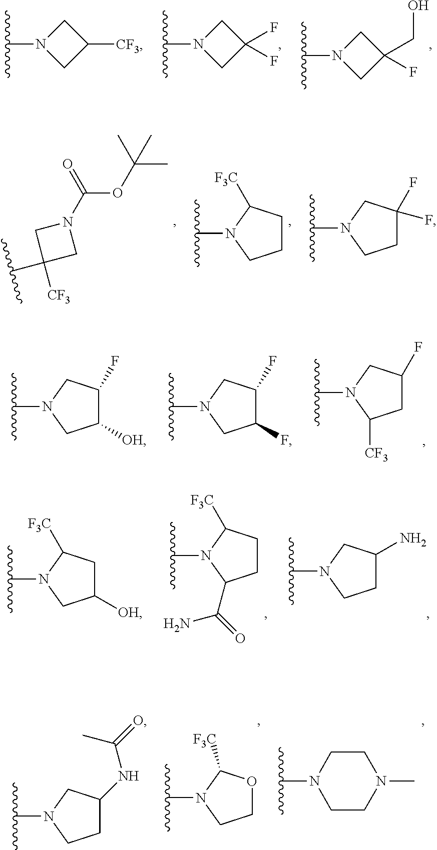

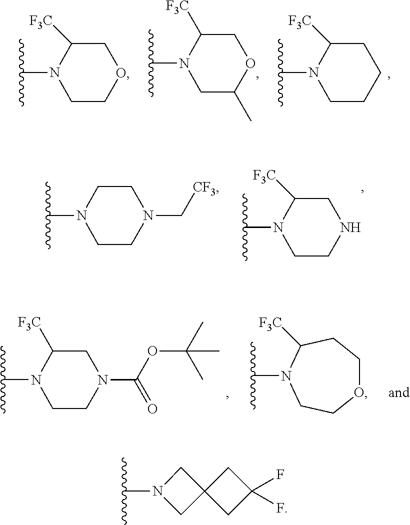

In some embodiments, for a compound or salt of Formula (I) or (I-a), R³ and R⁴ come together to form a saturated 4- to 8-membered heterocycle and the saturated 4- to 8-membered heterocycle is selected from azetidine, oxetane, pyrrolidine, pyrazolidine, imidazolidine, oxazolidine, tetrahydrofuran, tetrahydrothiophene, piperidine, piperazine, tetrahydropyran, morpholine, oxazepane, and azaspirol[3.3]heptane, any one of which is optionally substituted with one substituents independently selected from chloro, fluoro, —OR¹², —NO₂, and —CN; and C₁₋₃ alkyl optionally substituted with one or more substituents independently selected from chloro, fluoro, OR¹⁶, —NO₂, and —CN.

In some embodiments, for a compound or salt of Formula (I) or (I-a), R³ and R¹ come together to form a saturated 4- to 8-membered heterocycle and the saturated 4- to 8-membered heterocycle is selected from azetidine, oxetane, pyrrolidine, pyrazolidine, imidazolidine, oxazolidine, tetrahydrofuran, tetrahydrothiophene, piperidine, piperazine, tetrahydropyran, morpholine, oxazepane, and azaspiro[3.3]heptane, any one of which is optionally substituted with one substituents independently selected from chloro, fluoro, —OR¹⁶, —NO₂, —C(O)N(R¹⁶)₂, and —CN; and C₁₋₃ alkyl optionally substituted with one or more substituents independently selected from chloro, fluoro, OR⁶, —NO₂, and —CN.

In some embodiments, for a compound or salt of Formula (I) or (I-a), R³ and R⁴ of

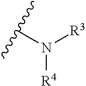

come together to form the saturated 4- to 8-membered heterocycle and

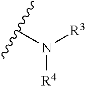

is selected from:

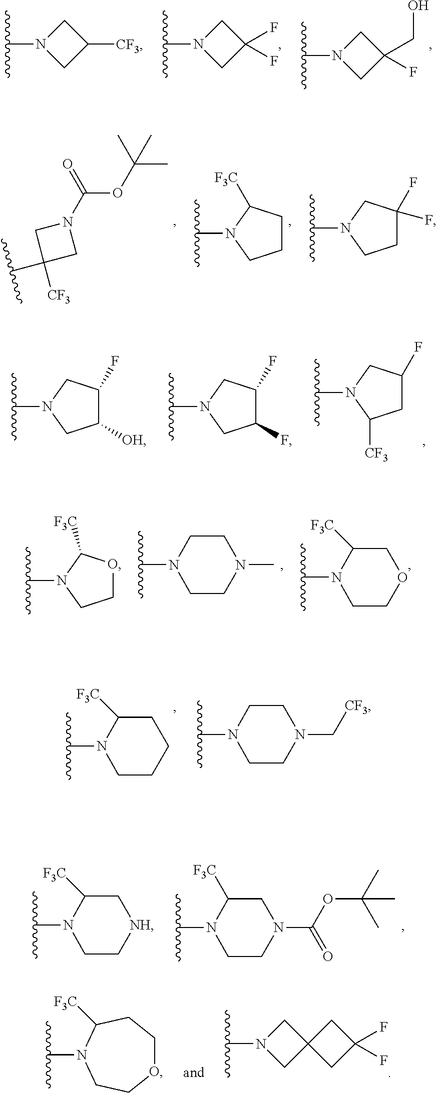

In some embodiments, for a compound or salt of Formula (I) or (I-a), R³ and R⁴ of

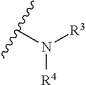

come together to form the saturated 4- to 8-membered heterocycle and

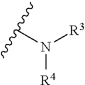

is selected from:

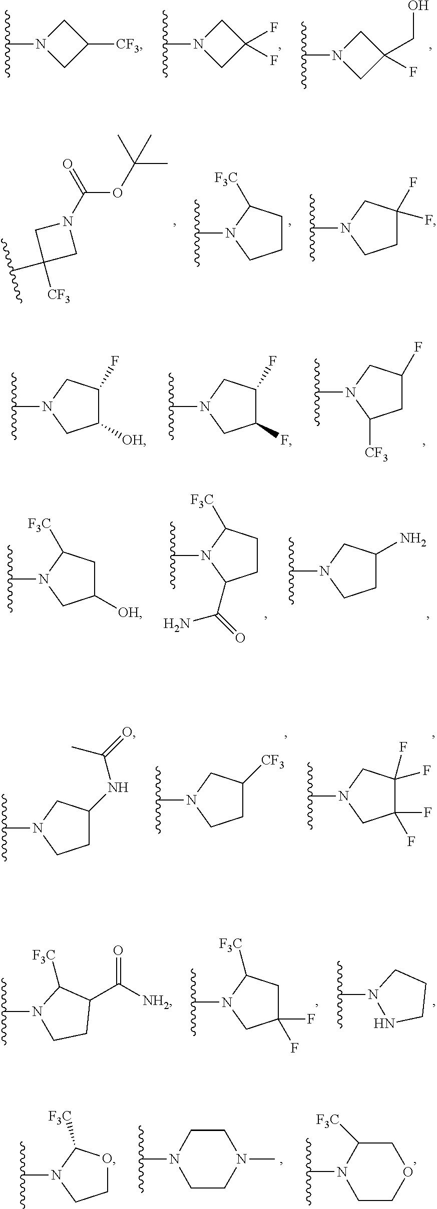

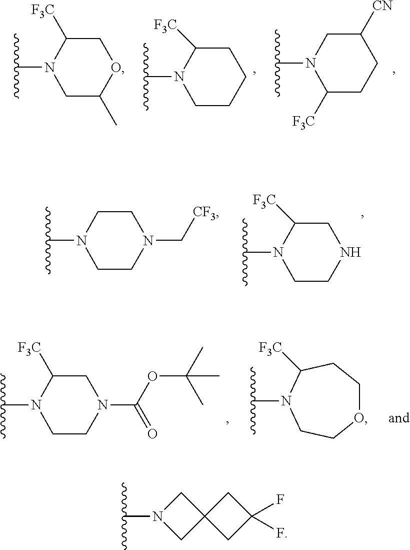

In some embodiments, for a compound or salt of Formula (I) or (I-a), the 4- to 12-membered heterocycle is an unsaturated 4- to 12-membered heterocycle any one of which is optionally substituted.

In some embodiments, for a compound or salt of Formula (I) or (I-a), R³ and R⁴ of

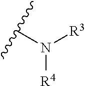

come together to form an unsaturated 4- to 12-membered heterocycle and

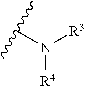

is represented by

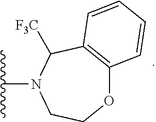 [SMILES: *N1CCOc2ccccc2C1C(F)(F)F]

In some embodiments, for a compound or salt of Formula (I) or (I-a),

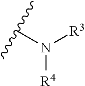

is selected

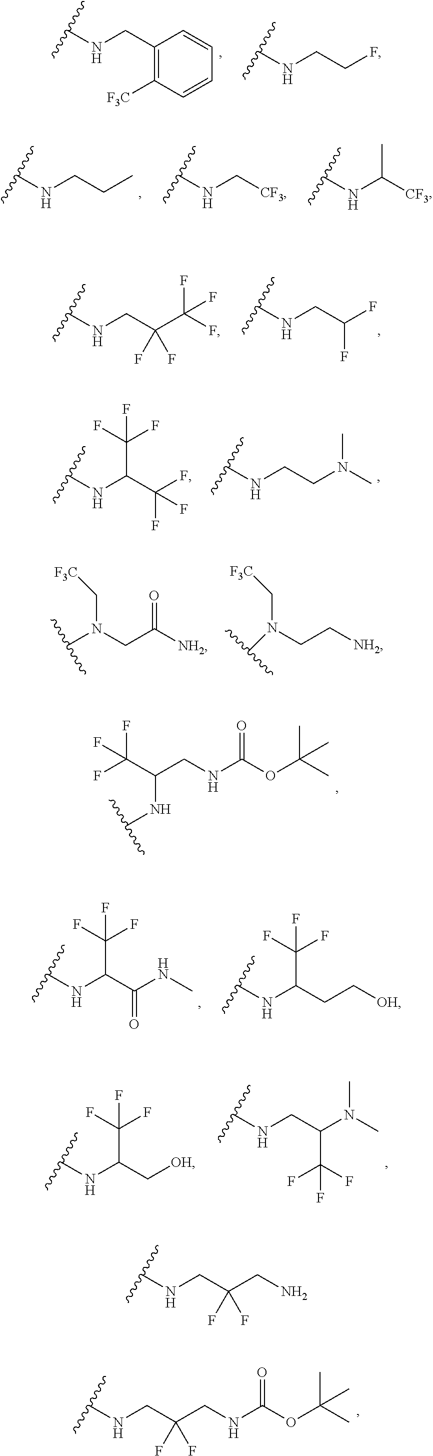

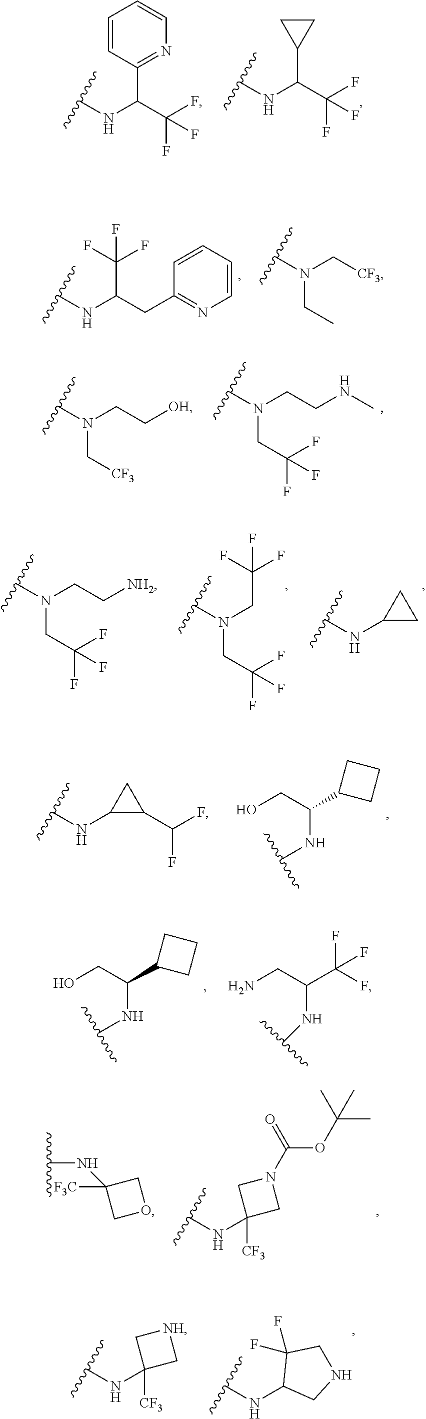

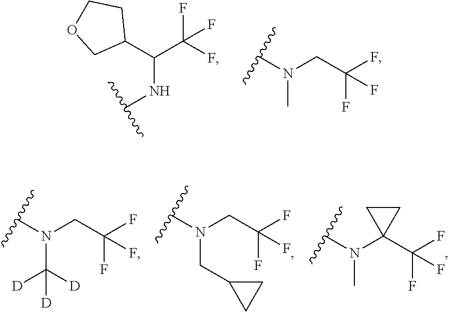

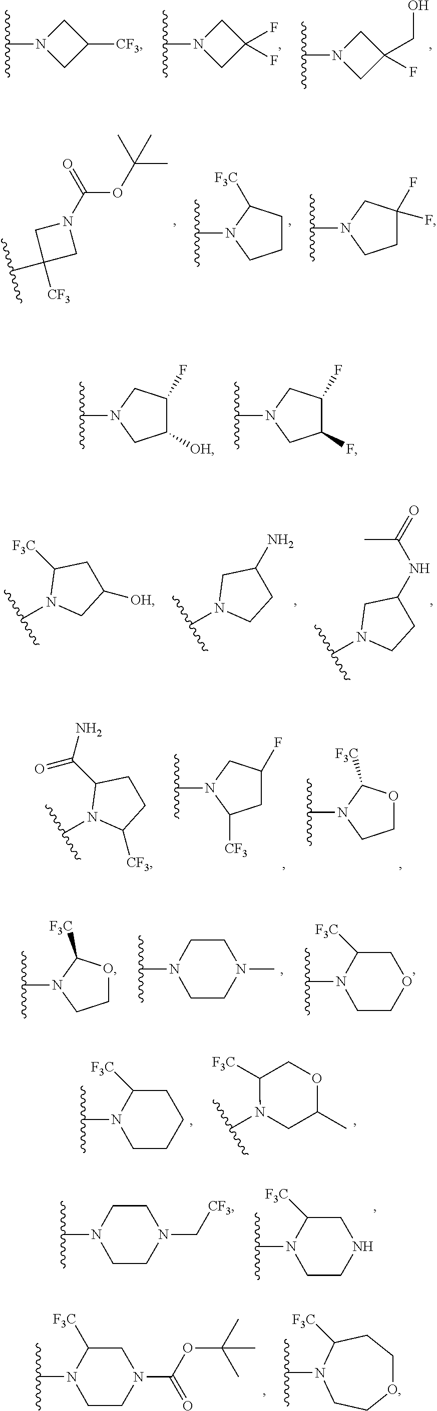

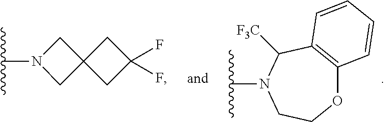 [SMILES: *N1CC2(C1)CC(F)(F)C2 ; *N1CCOc2ccccc2C1C(F)(F)F]

In some embodiments, for a compound or salt of Formula (I) or (I-a),

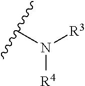

is selected from:

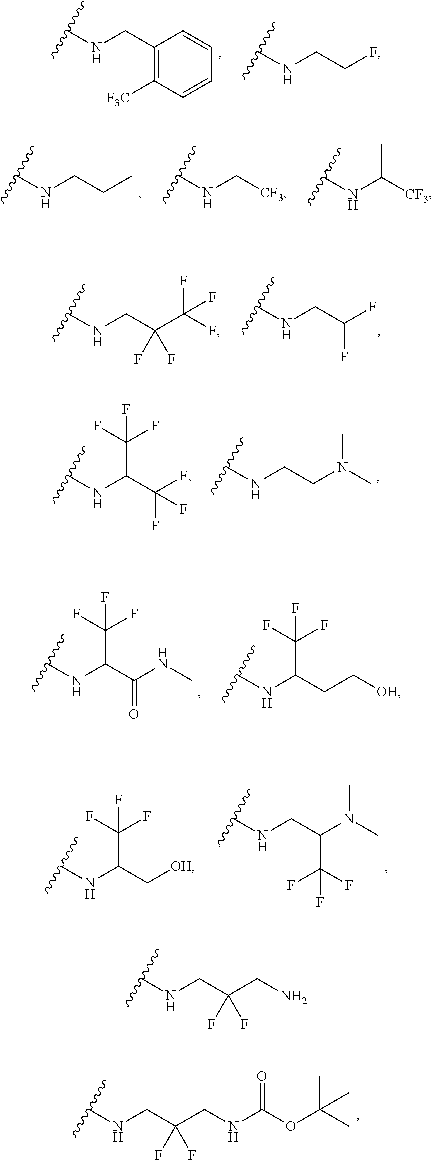

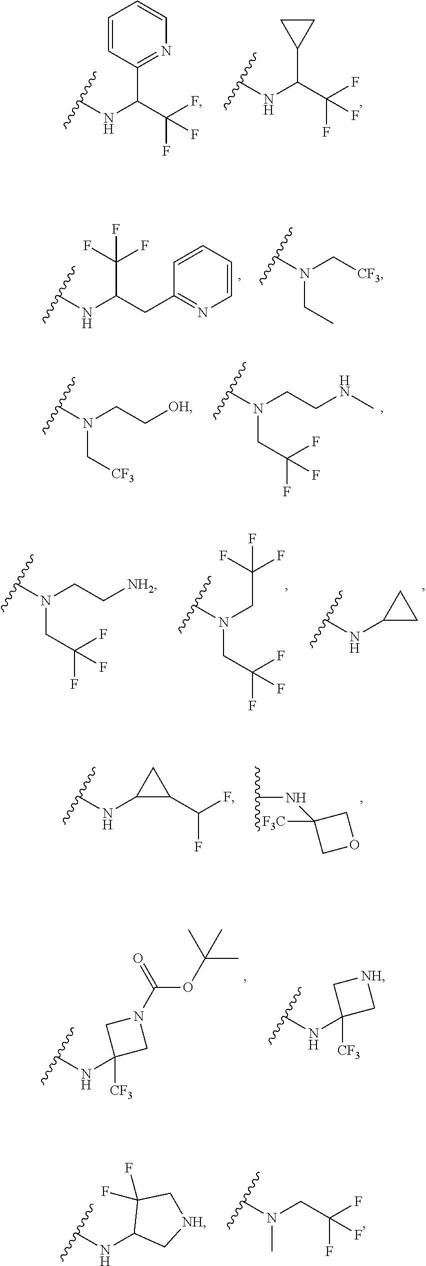

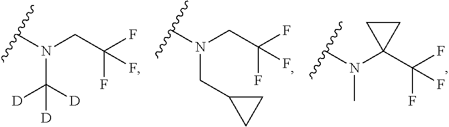

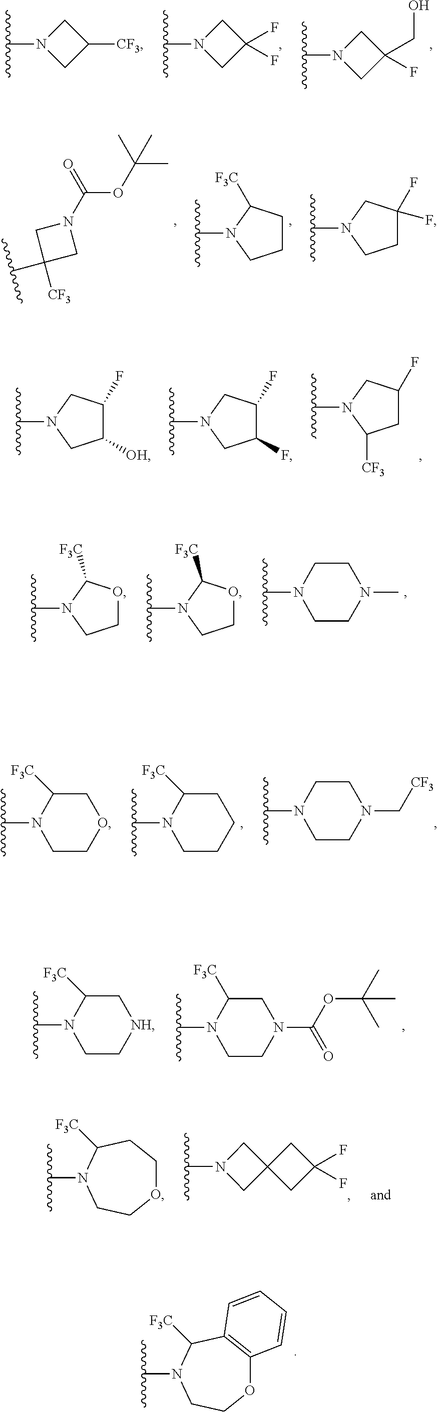

In another aspect, the present disclosure provides a compound represented by the structure of Formula (II):

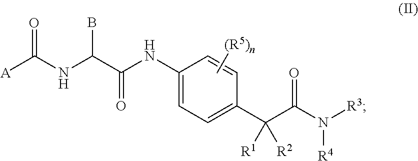

or a pharmaceutically acceptable salt thereof, wherein A, B, R¹¹, R², R³, R¹⁴, and n are as defined in Formula (I) or Formula (I-a), and R⁵ is selected from:
   halogen, —OR¹⁷, —SR¹¹, —N(R¹¹)₂, —C(O)R¹¹, —C(O)OR¹¹, —OC(O)R¹¹, —OC(O)N(R¹¹)₂, —C(O)N(R¹¹)₂, —N(R¹¹)C(O)R¹¹, —N(R¹¹)₇)C(O)OR¹¹, —N(R¹¹)C(O)N(R¹¹)₇)₂, —N(R¹¹)S(O)₂(R¹¹)₇), —S(O)R¹¹, —S(O)₂R⁴, —S(O)₂N(R¹⁷)₂, —NO₂, and —CN;
   C₁₋₆ alkyl and C₂₋₆ alkenyl each of which is optionally substituted with one or more substituents independently selected from halogen, —OR¹¹, —SR¹², —N(R¹¹)₂, —C(O)R¹¹, —S(═O)(=NR 17)R¹⁷, —C(O)OR¹¹, —OC(O)R¹¹, —OC(O)N(R⁷)₂, —C(O)N(R⁷)₂, —N(R¹⁷)C(O)R¹⁷, —N(R¹⁷)C(O)OR¹⁷, —N(R¹⁷)C(O)N(R¹⁷)₂, —N(R¹⁷)S(O)₂(R¹⁷), —S(O)R¹¹, —S(O)₂R¹¹, —S(O)₂N(R¹⁷)₂, —NO₂, ═O, and —CN; and C₃₋₁₀ carbocycle and 3- to 10 membered heterocycle optionally substituted with one or more substituents independently selected from halogen, —OR¹⁷, —SR¹⁶, —N(R¹⁴)₂, —C(O)R¹B, —C(O)OR¹⁷B, —OC(O)R^{17B}, —OC(O)N(R⁷)₂, —C(O)N(R^{1B})₂, —N(R^{B})C(O)R^{17B}, —N(R^{17B})C(O)OR¹⁴, —N(R¹⁴)C(O)N(R^{17B})₂, —N(R^{17B})S(O)₂(RB), —S(O)R¹⁴, —S(O)₂R^{17B}—S(O)₂N(R¹⁷)₂, —NO₂=0, and —CN.

In some embodiments, for the compound or salt of Formula (II), R⁵ is selected from:
halogen, —OR¹⁷, —SR¹⁷, —N(R¹⁷)₂, —C(O)R¹¹, —C(O)OR¹¹, —OC(O)R¹¹, —OC(O)N(R¹¹)₂, —C(O)N(R¹¹)₂, —N(R¹⁷)C(O)R¹⁷, —N(R¹⁷)C(O)OR¹¹, —N(R¹)C(O)N(R¹¹)₂, —N(R¹¹)S(O)₂(R¹⁷), —S(O)R¹¹, —S(O)₂R⁴, —S(O)₂N(R¹⁷)₂, —NO₂, and —CN; and
carbocycle and 3- to 10 membered heterocycle optionally substituted with one or more substituents independently selected from halogen, —OR^{17B}, —SR^{17B}, —N(R^{17B})₂, —C(O)R¹⁷, —C(O)OR¹⁷, —OC(O)R^{17B}, —OC(O)N(R⁷)₂, —C(O)N(R¹⁴)₂, —N(R¹¹)C(O)R¹—N(R¹⁷)C(O)OR^{B}, —N(R^{17B})C(O)N(R^{17B})₂, —N(R^{7B})S( )₂(R^{1 B}), —S(O)R^{17B}, —S(O)₂, S(O)₂N(R^{17B})₂, —NO₂, ═O, and —CN.

In some embodiments, for the compound or salt of Formula (II), R⁵ is selected from:
C₁₋₆ alkyl and C₂₋₆ alkenyl each of which is optionally substituted with one or more substituents independently selected from halogen, —OR¹⁷, —SR¹⁷, —N(R¹⁷)₂, —C(O)R¹⁷, —S(═O)(=NR¹⁷)R¹¹, —C(O)OR¹⁷, —OC(O)R¹⁷, —OC(O)N(R¹¹)₂, —C(O)N(R¹¹)₂, —N(R¹⁷)C(O)R¹⁷, —N(R¹¹)C(O)OR¹⁷, —N(R¹⁷)C(O)N(R¹⁷)₂, —N(R¹⁷)S(O)₂(R¹⁷), —S(O)R¹⁴, —S(O)₂R¹⁷, —S(O)₂N(R¹⁷)₂, —NO₂, ═O, and —CN; and
C₃₋₁₀ carbocycle and 3- to 10 membered heterocycle optionally substituted with one or more substituents independently selected from halogen, —OR^{1B}, —SR^{17B}, —N(R^{17B})₂, —C(O)R¹, —C(O)OR¹¹)_{H}, —OC(O)R¹¹)_{B}, —OC(O)N(R^{17B})₂, —C(O)N(R¹¹)_{17B})₂, —N(R^{17B})C(O)R¹¹)_{B}, —N(R¹⁷h)C(O)OR¹⁷, —N(R^{17B})C(O)N(R^{17B})₂, —N(R^{17B})S(O)₂(R^{17B}), —S(O)R^{17B}, —S(O)₂R^{17B}, —S(O)₂N(R^{17B})₂, —NO₂, ═O, and —CN.

In some embodiments, for the compound or salt of Formula (II), R⁵ is selected from C₃₋₁₀ carbocycle and 3- to 10 membered heterocycle optionally substituted with one or more substituents independently selected from halogen, —OR^{17B}, —SR^{17B}, —N(R^{17B})₂, —C(O)R^{M}, —C(O)OR^{17B}, —OC(O)R^{17B}, —OC(O)N(R^{17B})₂, —C(O)N(R^{17B})₂, —N(R^{17B})C(O)R^{17B}, —N(R^{17B})C(O)OR¹⁷, —N(R^{17B})C(O)N(R^{17B})₂,—N(R^{17B})S(O)₂(R^{17B}),—S(O)R^{17B}, —S(O)₂R^{, —S(O)}₂N(R¹¹)₁)₂, —NO₂, ═O, and —CN. In some embodiments, R⁵ is 3- to 10 membered heterocycle optionally substituted with one or more substituents independently selected from halogen, —OR¹⁷, —SR¹⁷B, —N(R^{17B})₂, —C(O)R¹⁷, —C(O)OR^{17B}, —OC(O)R^{1B}, —OC(O)N(R^{17B})₂, —C(O)N(R^{17B})₂, —N(R^{17B})C(O)R^{17B}, —N(R^{17B})C(O)OR¹⁷B, —N(R^{17B})C(O)N(R¹⁷8)₂, —N(R^{17B})S(O)₂(R^{17B}), —S(O)R^{17B}, —S(O)₂R^{17B}, —S(O)₂N(R⁷)₂, —NO₂, ═O, and —CN. In some embodiments, R⁵ is a 3- to 10 membered saturated heterocycle optionally substituted with one or more substituents independently selected from halogen, —OR^{17B}, —SR^{17B}, —N(R^{17B})₂, —C(O)R^{17B}, —C(O)OR^{17B}, —OC(O)R^{17B}, —OC(O)N(R^{17B})₂, —C(O)N(R^{17B})₂, —N(R^{17B})C(O)R^{17B}, —N(R^{17B})C(O)OR^{17B}, —N(R^{17B})C(O)N(R^{17B})₂, —N(R^{17B})S(O)₂(R^{17B}), —S(O)R^{17B}, —S(O)₂R^{17B}, —S(O)₂N(R^{17B})₂, —NO₂, ═O, and —CN. In some embodiments, R⁵ is selected from

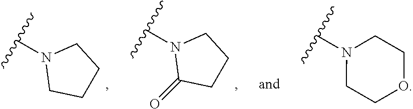 [SMILES: *N1CCCC1 ; *N1CCCC1=O ; *N1CCOCC1]

In some embodiments, R⁵ is

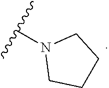 [SMILES: *N1CCCC1]

In some embodiments, R⁵ is

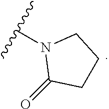 [SMILES: *N1CCCC1=O]

In some embodiments, R⁵ is

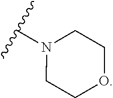 [SMILES: *N1CCOCC1]

In some aspects, the compound or salt of Formula I, (I-a), or II is selected from:

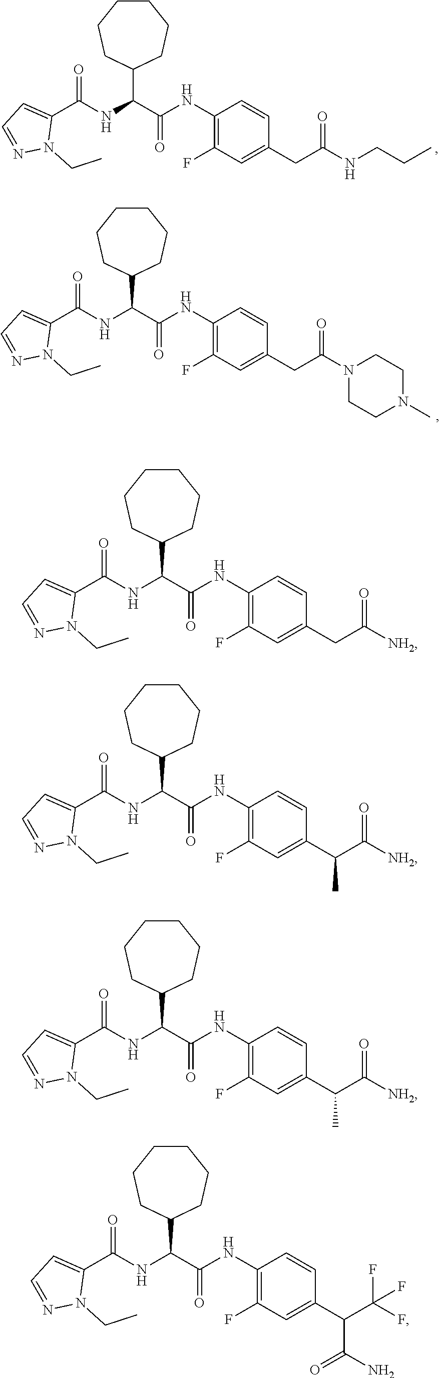 [SMILES: CCCNC(=O)Cc1ccc(NC(=O)[C@@H](NC(=O)c2ccnn2CC)C2CCCCCC2)c(F)c1 ; CCn1nccc1C(=O)N[C@H](C(=O)Nc1ccc(CC(=O)N2CCN(C)CC2)cc1F)C1CCCCCC1 ; CCn1nccc1C(=O)N[C@H](C(=O)Nc1ccc(CC(N)=O)cc1F)C1CCCCCC1 ; CCn1nccc1C(=O)N[C@H](C(=O)Nc1ccc([C@H](C)C(N)=O)cc1F)C1CCCCCC1 ; CCn1nccc1C(=O)N[C@H](C(=O)Nc1ccc([C@@H](C)C(N)=O)cc1F)C1CCCCCC1 ; CCn1nccc1C(=O)N[C@H](C(=O)Nc1ccc(C(C(N)=O)C(F)(F)F)cc1F)C1CCCCCC1]

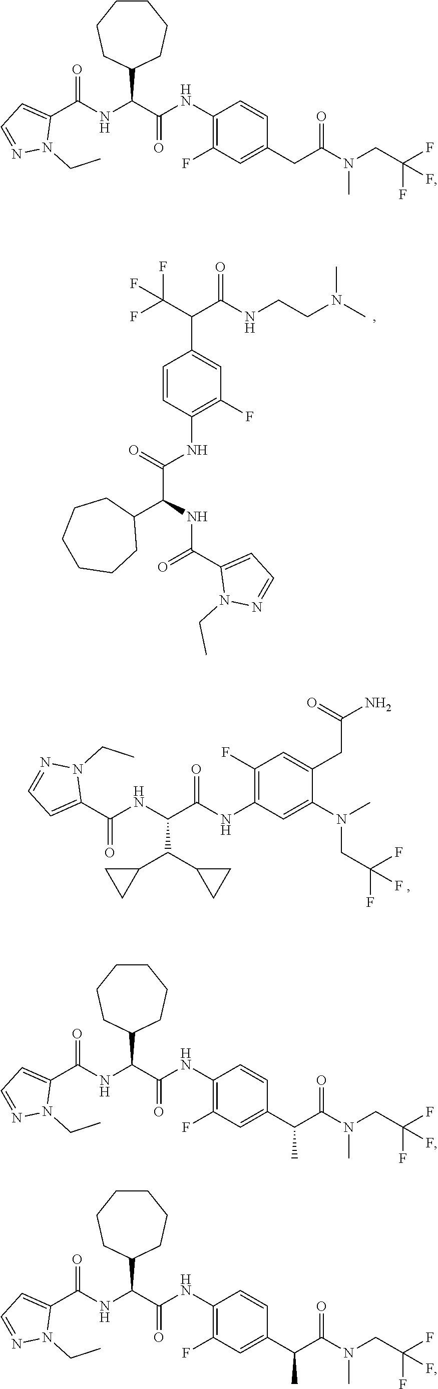 [SMILES: CCn1nccc1C(=O)N[C@H](C(=O)Nc1ccc(CC(=O)N(C)CC(F)(F)F)cc1F)C1CCCCCC1 ; CCn1nccc1C(=O)N[C@H](C(=O)Nc1ccc(C(C(=O)NCCN(C)C)C(F)(F)F)cc1F)C1CCCCCC1 ; CCn1nccc1C(=O)N[C@H](C(=O)Nc1cc(N(C)CC(F)(F)F)c(CC(N)=O)cc1F)C(C1CC1)C1CC1 ; CCn1nccc1C(=O)N[C@H](C(=O)Nc1ccc([C@@H](C)C(=O)N(C)CC(F)(F)F)cc1F)C1CCCCCC1 ; CCn1nccc1C(=O)N[C@H](C(=O)Nc1ccc([C@H](C)C(=O)N(C)CC(F)(F)F)cc1F)C1CCCCCC1]

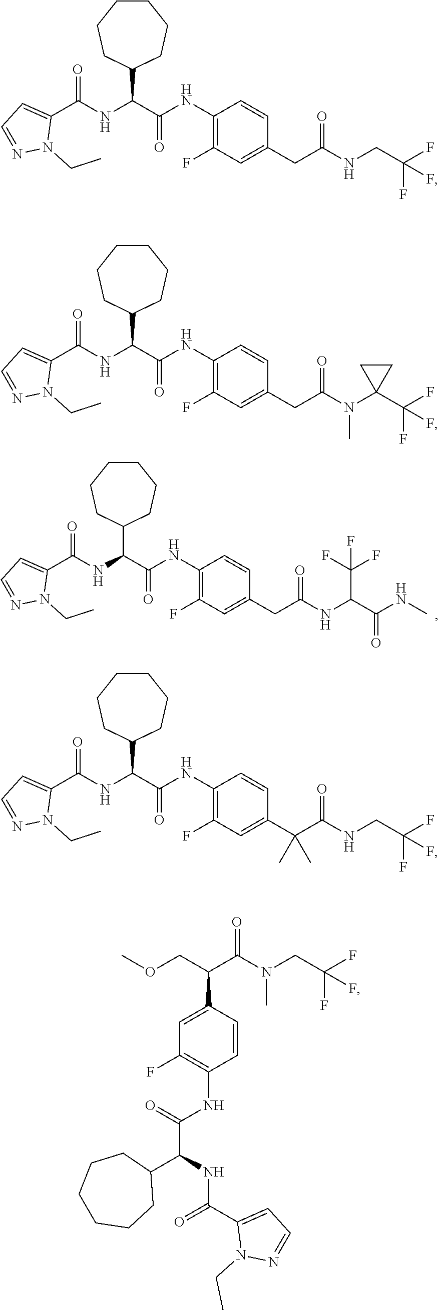 [SMILES: CCn1nccc1C(=O)N[C@H](C(=O)Nc1ccc(CC(=O)NCC(F)(F)F)cc1F)C1CCCCCC1 ; CCn1nccc1C(=O)N[C@H](C(=O)Nc1ccc(CC(=O)N(C)C2(C(F)(F)F)CC2)cc1F)C1CCCCCC1 ; CCn1nccc1C(=O)N[C@H](C(=O)Nc1ccc(CC(=O)NC(C(=O)NC)C(F)(F)F)cc1F)C1CCCCCC1 ; CCn1nccc1C(=O)N[C@H](C(=O)Nc1ccc(C(C)(C)C(=O)NCC(F)(F)F)cc1F)C1CCCCCC1 ; CCn1nccc1C(=O)N[C@H](C(=O)Nc1ccc([C@@H](COC)C(=O)N(C)CC(F)(F)F)cc1F)C1CCCCCC1]

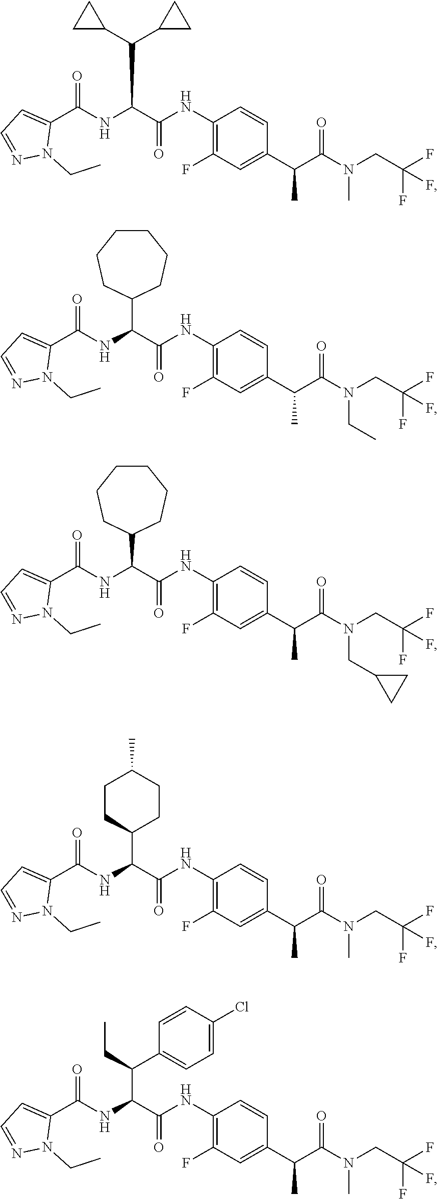 [SMILES: CCn1nccc1C(=O)N[C@H](C(=O)Nc1ccc([C@H](C)C(=O)N(C)CC(F)(F)F)cc1F)C(C1CC1)C1CC1 ; CCN(CC(F)(F)F)C(=O)[C@H](C)c1ccc(NC(=O)[C@@H](NC(=O)c2ccnn2CC)C2CCCCCC2)c(F)c1 ; CCn1nccc1C(=O)N[C@H](C(=O)Nc1ccc([C@H](C)C(=O)N(CC2CC2)CC(F)(F)F)cc1F)C1CCCCCC1 ; CCn1nccc1C(=O)N[C@H](C(=O)Nc1ccc([C@H](C)C(=O)N(C)CC(F)(F)F)cc1F)[C@H]1CC[C@H](C)CC1 ; CC[C@@H](c1ccc(Cl)cc1)[C@H](NC(=O)c1ccnn1CC)C(=O)Nc1ccc([C@H](C)C(=O)N(C)CC(F)(F)F)cc1F]

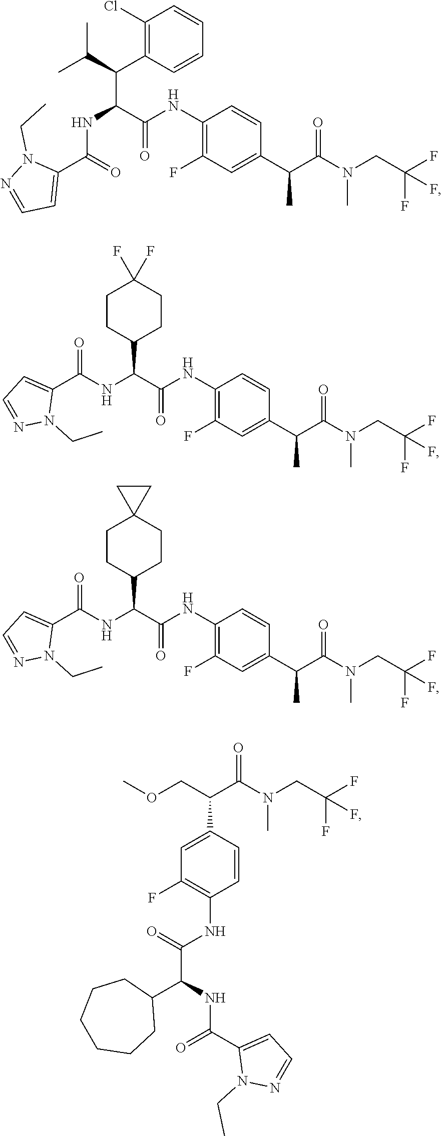 [SMILES: CCn1nccc1C(=O)N[C@H](C(=O)Nc1ccc([C@H](C)C(=O)N(C)CC(F)(F)F)cc1F)[C@H](c1ccccc1Cl)C(C)C ; CCn1nccc1C(=O)N[C@H](C(=O)Nc1ccc([C@H](C)C(=O)N(C)CC(F)(F)F)cc1F)C1CCC(F)(F)CC1 ; CCn1nccc1C(=O)N[C@H](C(=O)Nc1ccc([C@H](C)C(=O)N(C)CC(F)(F)F)cc1F)C1CCC2(CC1)CC2 ; CCn1nccc1C(=O)N[C@H](C(=O)Nc1ccc([C@H](COC)C(=O)N(C)CC(F)(F)F)cc1F)C1CCCCCC1]

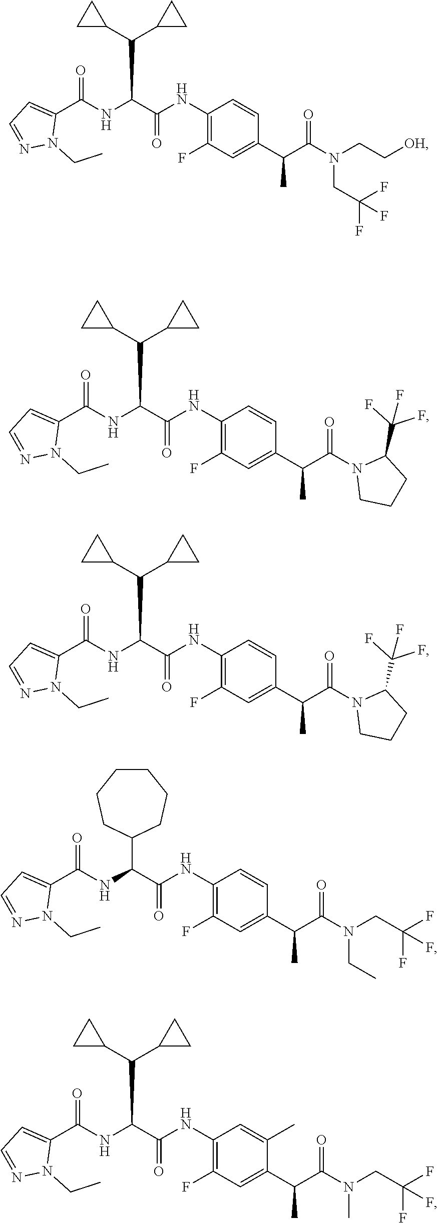 [SMILES: CCn1nccc1C(=O)N[C@H](C(=O)Nc1ccc([C@H](C)C(=O)N(CCO)CC(F)(F)F)cc1F)C(C1CC1)C1CC1 ; CCn1nccc1C(=O)N[C@H](C(=O)Nc1ccc([C@H](C)C(=O)N2CCC[C@@H]2C(F)(F)F)cc1F)C(C1CC1)C1CC1 ; CCn1nccc1C(=O)N[C@H](C(=O)Nc1ccc([C@H](C)C(=O)N2CCC[C@H]2C(F)(F)F)cc1F)C(C1CC1)C1CC1 ; CCN(CC(F)(F)F)C(=O)[C@@H](C)c1ccc(NC(=O)[C@@H](NC(=O)c2ccnn2CC)C2CCCCCC2)c(F)c1 ; CCn1nccc1C(=O)N[C@H](C(=O)Nc1cc(C)c([C@H](C)C(=O)N(C)CC(F)(F)F)cc1F)C(C1CC1)C1CC1]

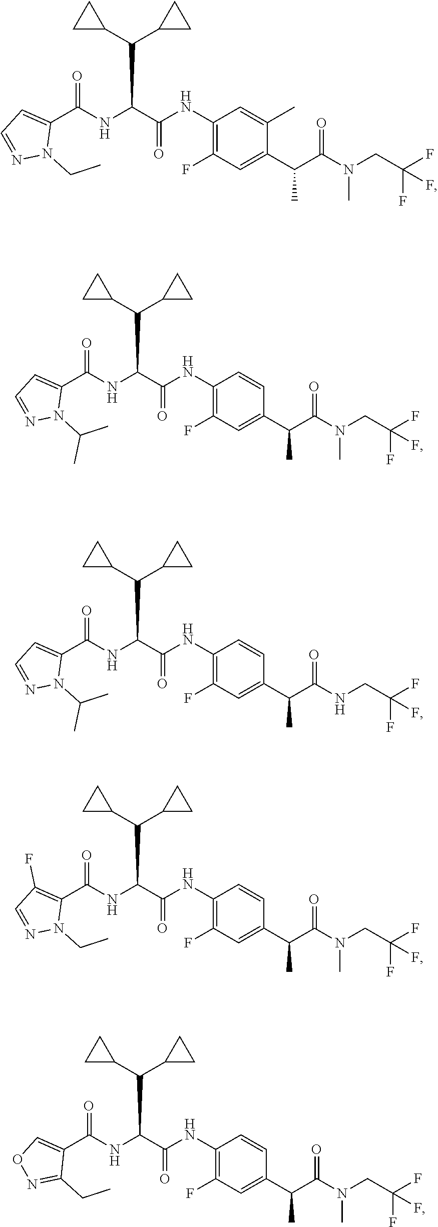 [SMILES: CCn1nccc1C(=O)N[C@H](C(=O)Nc1cc(C)c([C@@H](C)C(=O)N(C)CC(F)(F)F)cc1F)C(C1CC1)C1CC1 ; CC(C)n1nccc1C(=O)N[C@H](C(=O)Nc1ccc([C@H](C)C(=O)N(C)CC(F)(F)F)cc1F)C(C1CC1)C1CC1 ; CC(C)n1nccc1C(=O)N[C@H](C(=O)Nc1ccc([C@H](C)C(=O)NCC(F)(F)F)cc1F)C(C1CC1)C1CC1 ; CCn1ncc(F)c1C(=O)N[C@H](C(=O)Nc1ccc([C@H](C)C(=O)N(C)CC(F)(F)F)cc1F)C(C1CC1)C1CC1 ; CCc1nocc1C(=O)N[C@H](C(=O)Nc1ccc([C@H](C)C(=O)N(C)CC(F)(F)F)cc1F)C(C1CC1)C1CC1]

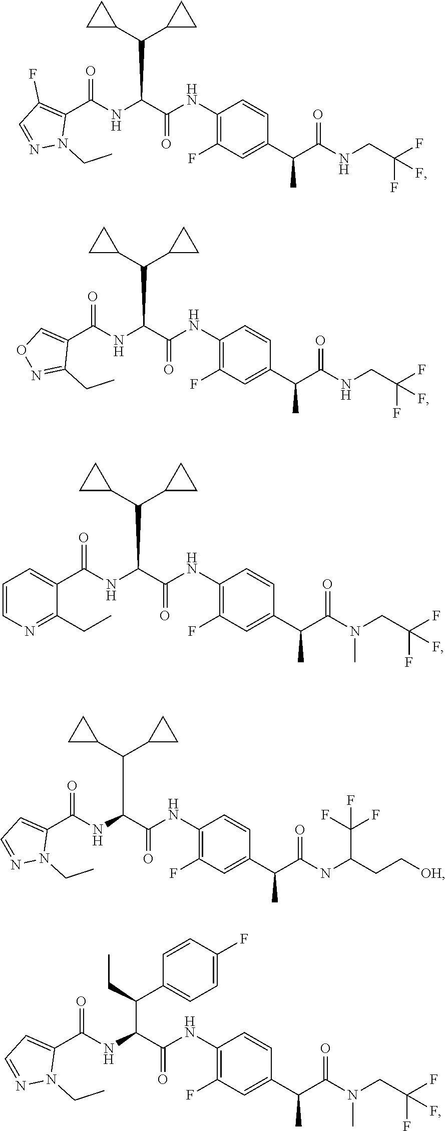 [SMILES: CCn1ncc(F)c1C(=O)N[C@H](C(=O)Nc1ccc([C@H](C)C(=O)NCC(F)(F)F)cc1F)C(C1CC1)C1CC1 ; CCc1nocc1C(=O)N[C@H](C(=O)Nc1ccc([C@H](C)C(=O)NCC(F)(F)F)cc1F)C(C1CC1)C1CC1 ; CCc1ncccc1C(=O)N[C@H](C(=O)Nc1ccc([C@H](C)C(=O)N(C)CC(F)(F)F)cc1F)C(C1CC1)C1CC1 ; CCn1nccc1C(=O)N[C@H](C(=O)Nc1ccc([C@H](C)C(=O)NC(CCO)C(F)(F)F)cc1F)C(C1CC1)C1CC1 ; CC[C@@H](c1ccc(F)cc1)[C@H](NC(=O)c1ccnn1CC)C(=O)Nc1ccc([C@H](C)C(=O)N(C)CC(F)(F)F)cc1F]

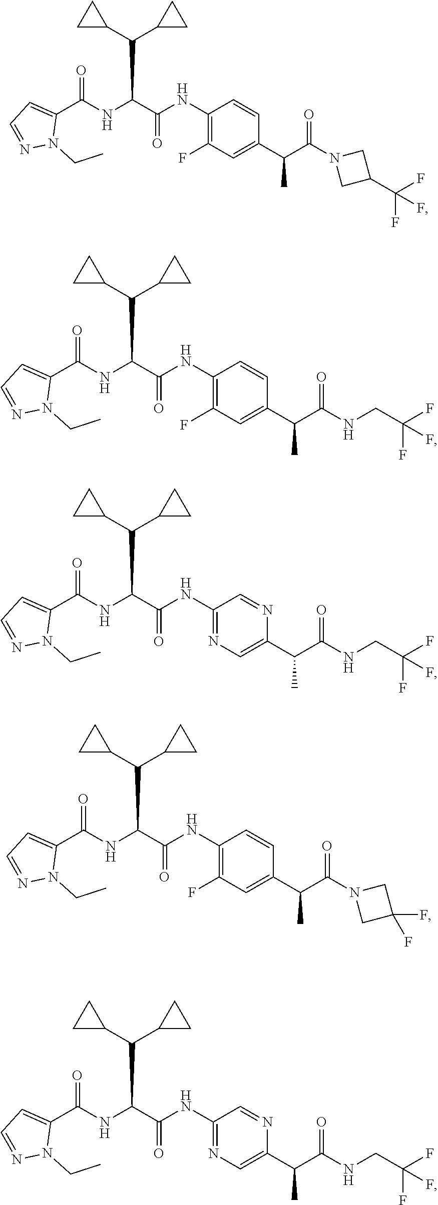 [SMILES: CCn1nccc1C(=O)N[C@H](C(=O)Nc1ccc([C@H](C)C(=O)N2CC(C(F)(F)F)C2)cc1F)C(C1CC1)C1CC1 ; CCn1nccc1C(=O)N[C@H](C(=O)Nc1ccc([C@H](C)C(=O)NCC(F)(F)F)cc1F)C(C1CC1)C1CC1 ; CCn1nccc1C(=O)N[C@H](C(=O)Nc1cnc([C@@H](C)C(=O)NCC(F)(F)F)cn1)C(C1CC1)C1CC1 ; CCn1nccc1C(=O)N[C@H](C(=O)Nc1ccc([C@H](C)C(=O)N2CC(F)(F)C2)cc1F)C(C1CC1)C1CC1 ; CCn1nccc1C(=O)N[C@H](C(=O)Nc1cnc([C@H](C)C(=O)NCC(F)(F)F)cn1)C(C1CC1)C1CC1]

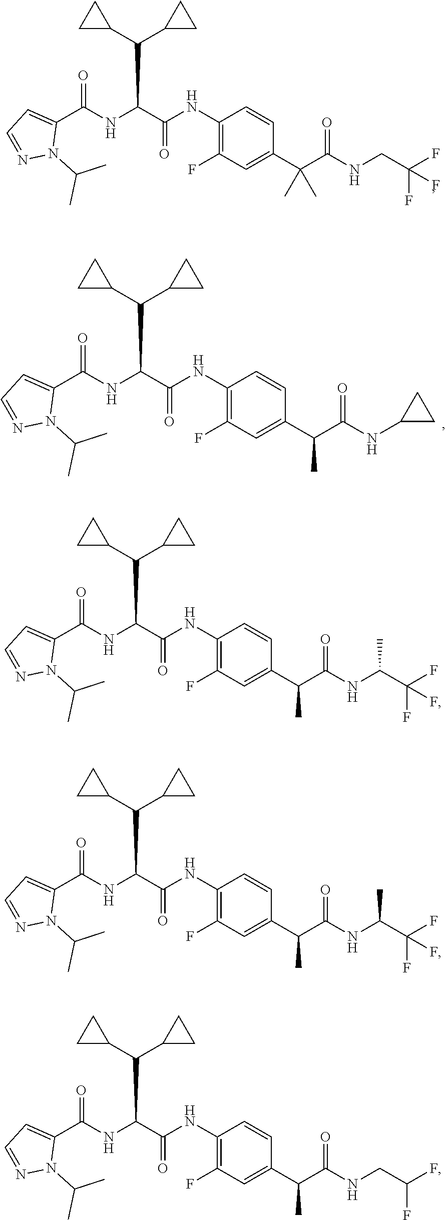 [SMILES: CC(C)n1nccc1C(=O)N[C@H](C(=O)Nc1ccc(C(C)(C)C(=O)NCC(F)(F)F)cc1F)C(C1CC1)C1CC1 ; CC(C)n1nccc1C(=O)N[C@H](C(=O)Nc1ccc([C@H](C)C(=O)NC2CC2)cc1F)C(C1CC1)C1CC1 ; CC(C)n1nccc1C(=O)N[C@H](C(=O)Nc1ccc([C@H](C)C(=O)N[C@H](C)C(F)(F)F)cc1F)C(C1CC1)C1CC1 ; CC(C)n1nccc1C(=O)N[C@H](C(=O)Nc1ccc([C@H](C)C(=O)N[C@@H](C)C(F)(F)F)cc1F)C(C1CC1)C1CC1 ; CC(C)n1nccc1C(=O)N[C@H](C(=O)Nc1ccc([C@H](C)C(=O)NCC(F)F)cc1F)C(C1CC1)C1CC1]

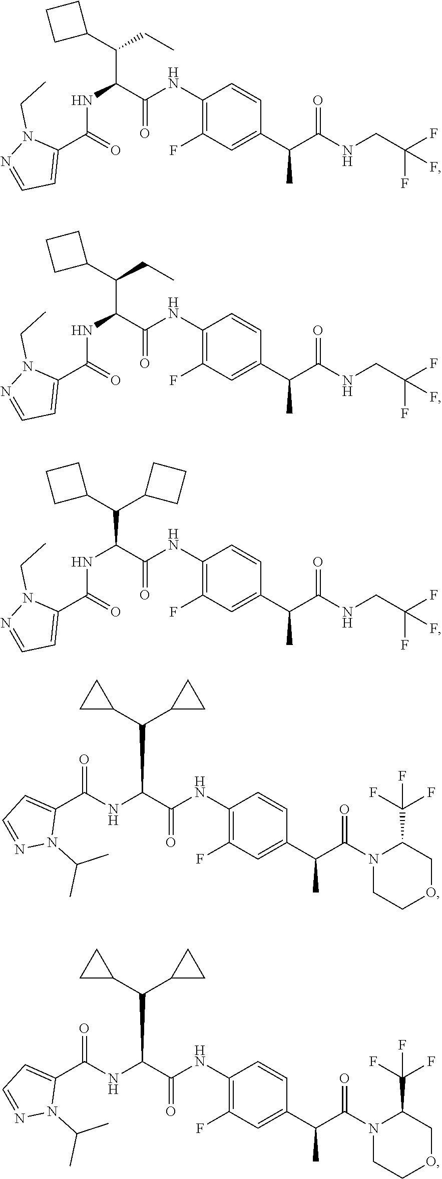 [SMILES: CC[C@@H](C1CCC1)[C@H](NC(=O)c1ccnn1CC)C(=O)Nc1ccc([C@H](C)C(=O)NCC(F)(F)F)cc1F ; CC[C@H](C1CCC1)[C@H](NC(=O)c1ccnn1CC)C(=O)Nc1ccc([C@H](C)C(=O)NCC(F)(F)F)cc1F ; CCn1nccc1C(=O)N[C@H](C(=O)Nc1ccc([C@H](C)C(=O)NCC(F)(F)F)cc1F)C(C1CCC1)C1CCC1 ; CC(C)n1nccc1C(=O)N[C@H](C(=O)Nc1ccc([C@H](C)C(=O)N2CCOC[C@H]2C(F)(F)F)cc1F)C(C1CC1)C1CC1 ; CC(C)n1nccc1C(=O)N[C@H](C(=O)Nc1ccc([C@H](C)C(=O)N2CCOC[C@@H]2C(F)(F)F)cc1F)C(C1CC1)C1CC1]

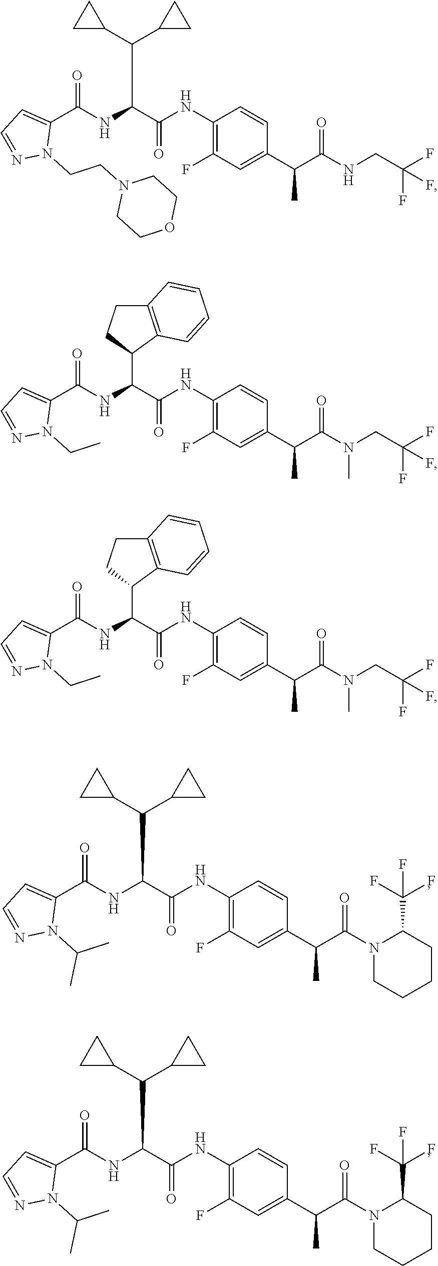 [SMILES: C[C@H](C(=O)NCC(F)(F)F)c1ccc(NC(=O)[C@@H](NC(=O)c2ccnn2CCN2CCOCC2)C(C2CC2)C2CC2)c(F)c1 ; CCn1nccc1C(=O)N[C@H](C(=O)Nc1ccc([C@H](C)C(=O)N(C)CC(F)(F)F)cc1F)[C@H]1CCc2ccccc21 ; CCn1nccc1C(=O)N[C@H](C(=O)Nc1ccc([C@H](C)C(=O)N(C)CC(F)(F)F)cc1F)[C@@H]1CCc2ccccc21 ; CC(C)n1nccc1C(=O)N[C@H](C(=O)Nc1ccc([C@H](C)C(=O)N2CCCC[C@H]2C(F)(F)F)cc1F)C(C1CC1)C1CC1 ; CC(C)n1nccc1C(=O)N[C@H](C(=O)Nc1ccc([C@H](C)C(=O)N2CCCC[C@@H]2C(F)(F)F)cc1F)C(C1CC1)C1CC1]

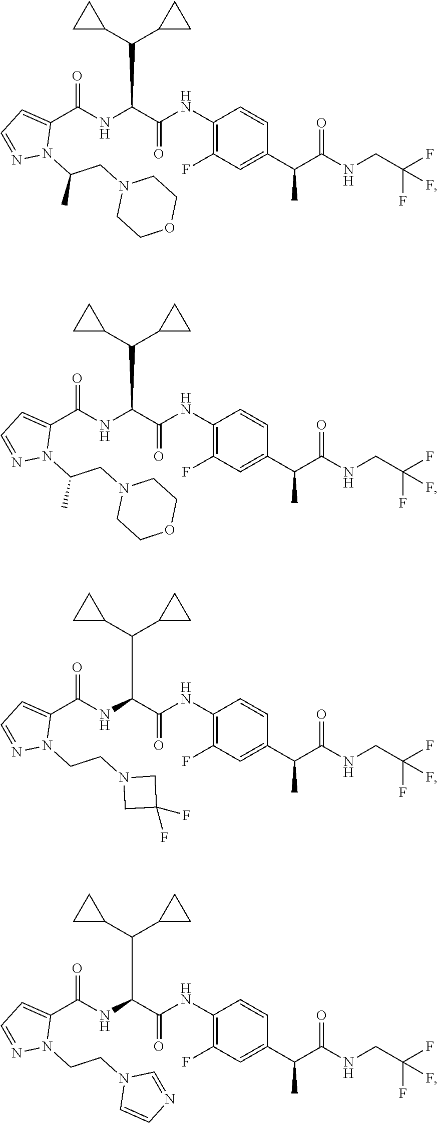 [SMILES: C[C@H](C(=O)NCC(F)(F)F)c1ccc(NC(=O)[C@@H](NC(=O)c2ccnn2[C@H](C)CN2CCOCC2)C(C2CC2)C2CC2)c(F)c1 ; C[C@H](C(=O)NCC(F)(F)F)c1ccc(NC(=O)[C@@H](NC(=O)c2ccnn2[C@@H](C)CN2CCOCC2)C(C2CC2)C2CC2)c(F)c1 ; C[C@H](C(=O)NCC(F)(F)F)c1ccc(NC(=O)[C@@H](NC(=O)c2ccnn2CCN2CC(F)(F)C2)C(C2CC2)C2CC2)c(F)c1 ; C[C@H](C(=O)NCC(F)(F)F)c1ccc(NC(=O)[C@@H](NC(=O)c2ccnn2CCn2ccnc2)C(C2CC2)C2CC2)c(F)c1]

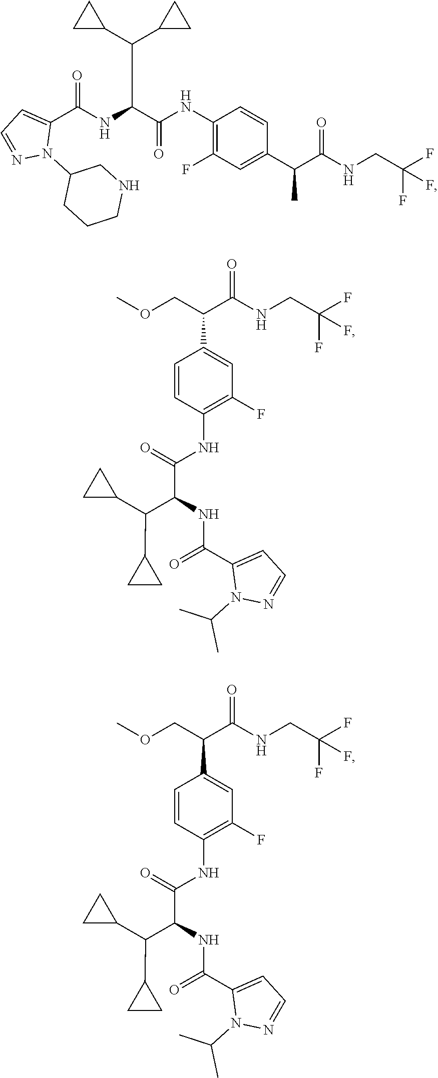

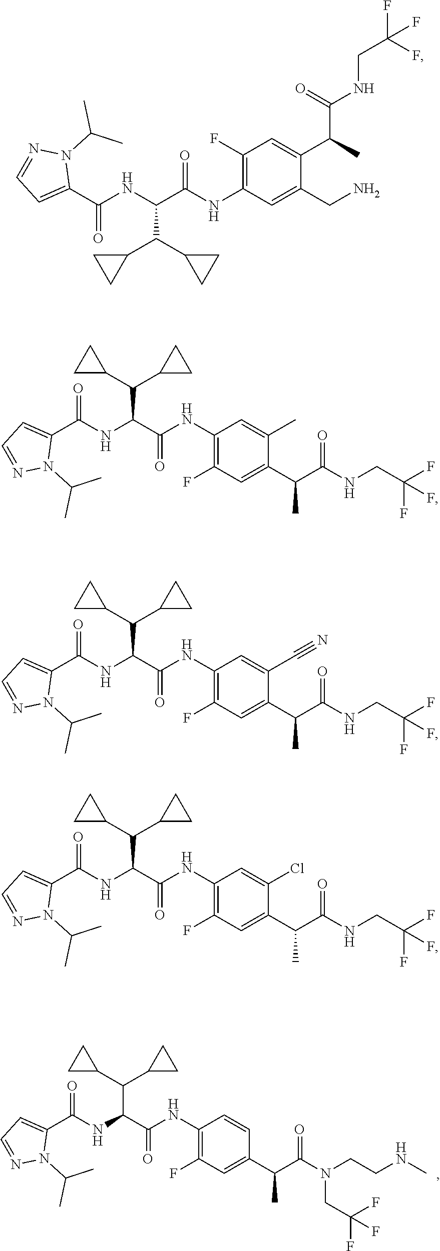 [SMILES: CC(C)n1nccc1C(=O)N[C@H](C(=O)Nc1cc(CN)c([C@H](C)C(=O)NCC(F)(F)F)cc1F)C(C1CC1)C1CC1 ; Cc1cc(NC(=O)[C@@H](NC(=O)c2ccnn2C(C)C)C(C2CC2)C2CC2)c(F)cc1[C@H](C)C(=O)NCC(F)(F)F ; CC(C)n1nccc1C(=O)N[C@H](C(=O)Nc1cc(C#N)c([C@H](C)C(=O)NCC(F)(F)F)cc1F)C(C1CC1)C1CC1 ; CC(C)n1nccc1C(=O)N[C@H](C(=O)Nc1cc(Cl)c([C@@H](C)C(=O)NCC(F)(F)F)cc1F)C(C1CC1)C1CC1 ; CNCCN(CC(F)(F)F)C(=O)[C@@H](C)c1ccc(NC(=O)[C@@H](NC(=O)c2ccnn2C(C)C)C(C2CC2)C2CC2)c(F)c1]

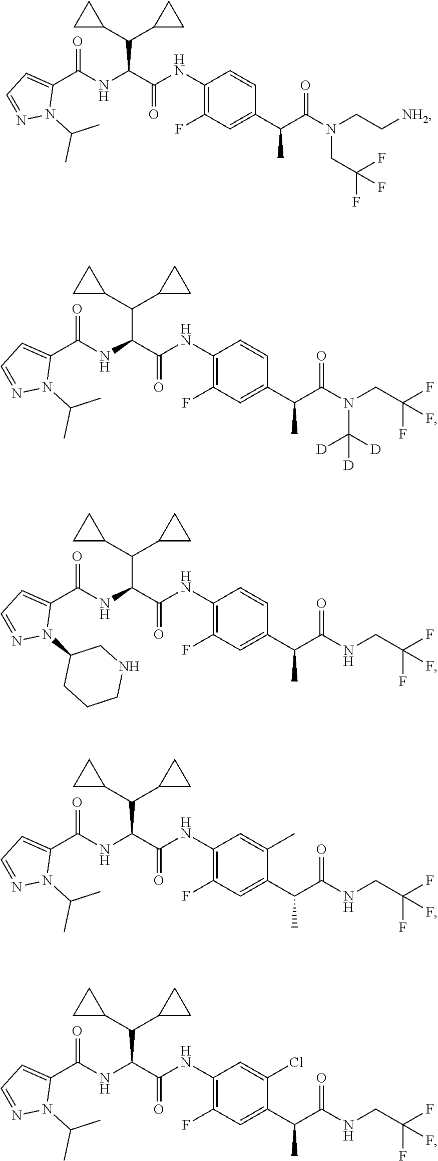 [SMILES: CC(C)n1nccc1C(=O)N[C@H](C(=O)Nc1ccc([C@H](C)C(=O)N(CCN)CC(F)(F)F)cc1F)C(C1CC1)C1CC1 ; [2H]C([2H])([2H])N(CC(F)(F)F)C(=O)[C@@H](C)c1ccc(NC(=O)[C@@H](NC(=O)c2ccnn2C(C)C)C(C2CC2)C2CC2)c(F)c1 ; C[C@H](C(=O)NCC(F)(F)F)c1ccc(NC(=O)[C@@H](NC(=O)c2ccnn2[C@@H]2CCCNC2)C(C2CC2)C2CC2)c(F)c1 ; Cc1cc(NC(=O)[C@@H](NC(=O)c2ccnn2C(C)C)C(C2CC2)C2CC2)c(F)cc1[C@@H](C)C(=O)NCC(F)(F)F ; CC(C)n1nccc1C(=O)N[C@H](C(=O)Nc1cc(Cl)c([C@H](C)C(=O)NCC(F)(F)F)cc1F)C(C1CC1)C1CC1]

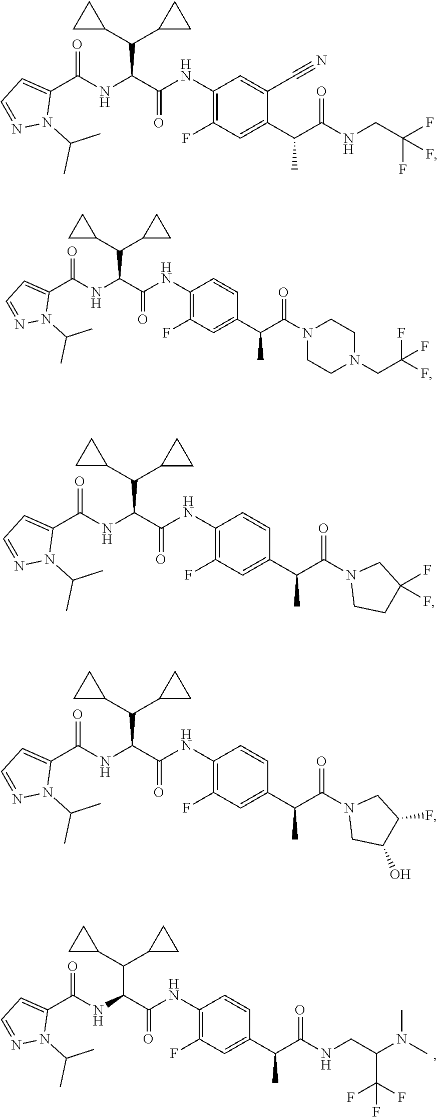 [SMILES: CC(C)n1nccc1C(=O)N[C@H](C(=O)Nc1cc(C#N)c([C@@H](C)C(=O)NCC(F)(F)F)cc1F)C(C1CC1)C1CC1 ; CC(C)n1nccc1C(=O)N[C@H](C(=O)Nc1ccc([C@H](C)C(=O)N2CCN(CC(F)(F)F)CC2)cc1F)C(C1CC1)C1CC1 ; CC(C)n1nccc1C(=O)N[C@H](C(=O)Nc1ccc([C@H](C)C(=O)N2CCC(F)(F)C2)cc1F)C(C1CC1)C1CC1 ; CC(C)n1nccc1C(=O)N[C@H](C(=O)Nc1ccc([C@H](C)C(=O)N2C[C@@H](O)[C@@H](F)C2)cc1F)C(C1CC1)C1CC1 ; CC(C)n1nccc1C(=O)N[C@H](C(=O)Nc1ccc([C@H](C)C(=O)NCC(N(C)C)C(F)(F)F)cc1F)C(C1CC1)C1CC1]

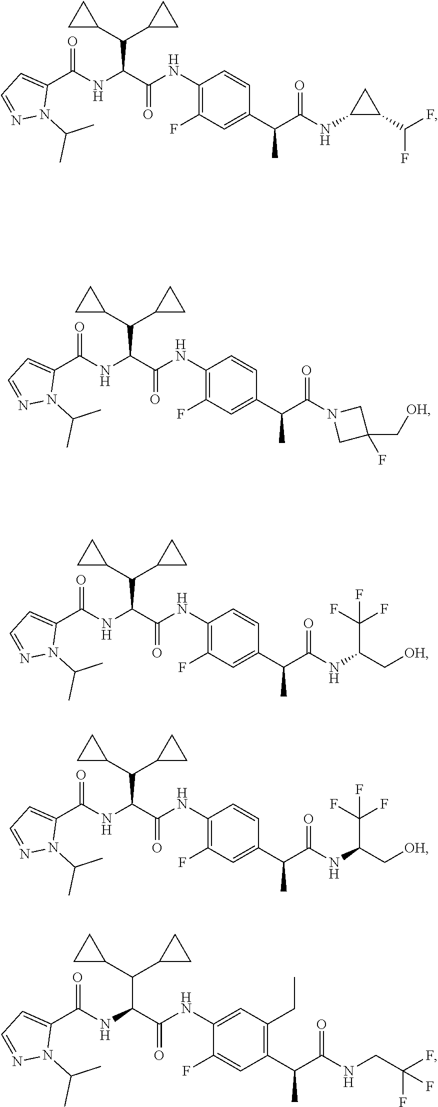 [SMILES: CC(C)n1nccc1C(=O)N[C@H](C(=O)Nc1ccc([C@H](C)C(=O)N[C@@H]2C[C@@H]2C(F)F)cc1F)C(C1CC1)C1CC1 ; CC(C)n1nccc1C(=O)N[C@H](C(=O)Nc1ccc([C@H](C)C(=O)N2CC(F)(CO)C2)cc1F)C(C1CC1)C1CC1 ; CC(C)n1nccc1C(=O)N[C@H](C(=O)Nc1ccc([C@H](C)C(=O)N[C@@H](CO)C(F)(F)F)cc1F)C(C1CC1)C1CC1 ; CC(C)n1nccc1C(=O)N[C@H](C(=O)Nc1ccc([C@H](C)C(=O)N[C@H](CO)C(F)(F)F)cc1F)C(C1CC1)C1CC1 ; CCc1cc(NC(=O)[C@@H](NC(=O)c2ccnn2C(C)C)C(C2CC2)C2CC2)c(F)cc1[C@H](C)C(=O)NCC(F)(F)F]

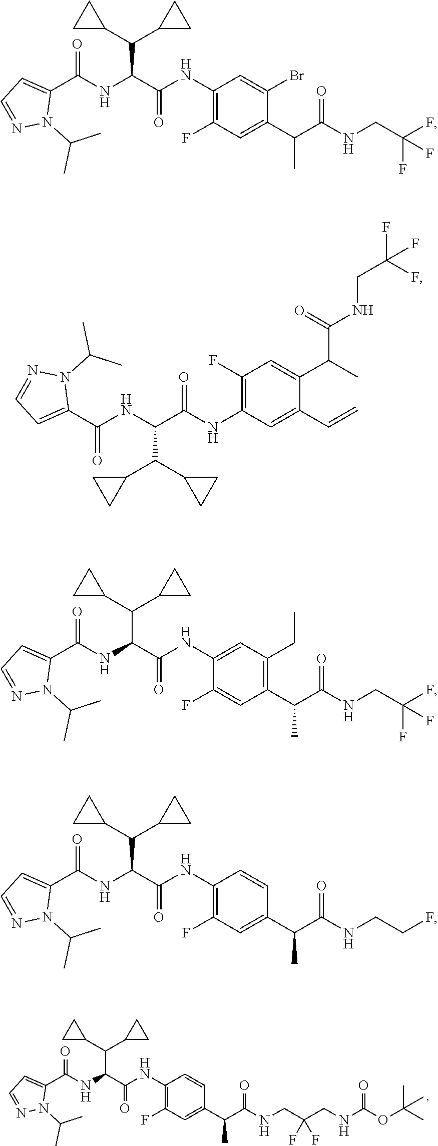 [SMILES: CC(C(=O)NCC(F)(F)F)c1cc(F)c(NC(=O)[C@@H](NC(=O)c2ccnn2C(C)C)C(C2CC2)C2CC2)cc1Br ; C=Cc1cc(NC(=O)[C@@H](NC(=O)c2ccnn2C(C)C)C(C2CC2)C2CC2)c(F)cc1C(C)C(=O)NCC(F)(F)F ; CCc1cc(NC(=O)[C@@H](NC(=O)c2ccnn2C(C)C)C(C2CC2)C2CC2)c(F)cc1[C@@H](C)C(=O)NCC(F)(F)F ; CC(C)n1nccc1C(=O)N[C@H](C(=O)Nc1ccc([C@H](C)C(=O)NCCF)cc1F)C(C1CC1)C1CC1 ; CC(C)n1nccc1C(=O)N[C@H](C(=O)Nc1ccc([C@H](C)C(=O)NCC(F)(F)CNC(=O)OC(C)(C)C)cc1F)C(C1CC1)C1CC1]

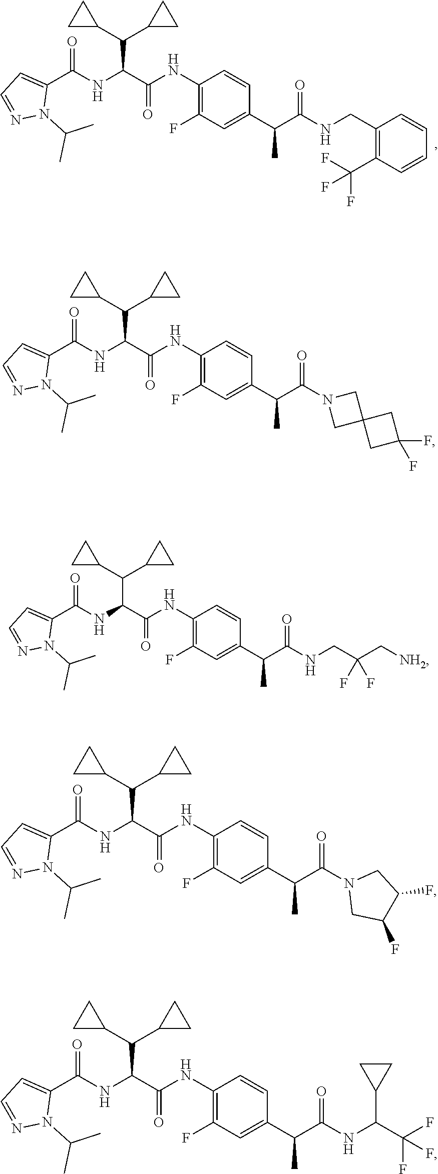 [SMILES: CC(C)n1nccc1C(=O)N[C@H](C(=O)Nc1ccc([C@H](C)C(=O)NCc2ccccc2C(F)(F)F)cc1F)C(C1CC1)C1CC1 ; CC(C)n1nccc1C(=O)N[C@H](C(=O)Nc1ccc([C@H](C)C(=O)N2CC3(C2)CC(F)(F)C3)cc1F)C(C1CC1)C1CC1 ; CC(C)n1nccc1C(=O)N[C@H](C(=O)Nc1ccc([C@H](C)C(=O)NCC(F)(F)CN)cc1F)C(C1CC1)C1CC1 ; CC(C)n1nccc1C(=O)N[C@H](C(=O)Nc1ccc([C@H](C)C(=O)N2C[C@H](F)[C@@H](F)C2)cc1F)C(C1CC1)C1CC1 ; CC(C)n1nccc1C(=O)N[C@H](C(=O)Nc1ccc([C@H](C)C(=O)NC(C2CC2)C(F)(F)F)cc1F)C(C1CC1)C1CC1]

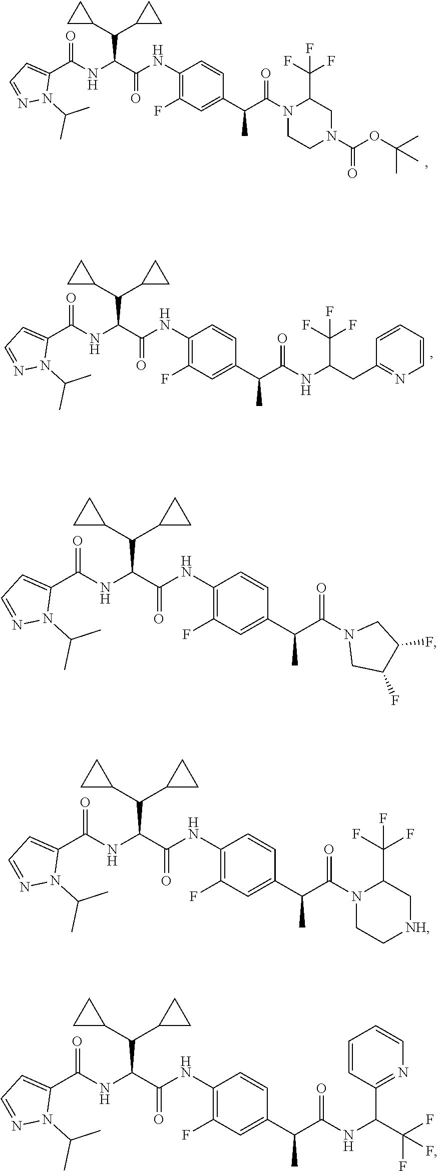 [SMILES: CC(C)n1nccc1C(=O)N[C@H](C(=O)Nc1ccc([C@H](C)C(=O)N2CCN(C(=O)OC(C)(C)C)CC2C(F)(F)F)cc1F)C(C1CC1)C1CC1 ; CC(C)n1nccc1C(=O)N[C@H](C(=O)Nc1ccc([C@H](C)C(=O)NC(Cc2ccccn2)C(F)(F)F)cc1F)C(C1CC1)C1CC1 ; CC(C)n1nccc1C(=O)N[C@H](C(=O)Nc1ccc([C@H](C)C(=O)N2C[C@@H](F)[C@@H](F)C2)cc1F)C(C1CC1)C1CC1 ; CC(C)n1nccc1C(=O)N[C@H](C(=O)Nc1ccc([C@H](C)C(=O)N2CCNCC2C(F)(F)F)cc1F)C(C1CC1)C1CC1 ; CC(C)n1nccc1C(=O)N[C@H](C(=O)Nc1ccc([C@H](C)C(=O)NC(c2ccccn2)C(F)(F)F)cc1F)C(C1CC1)C1CC1]

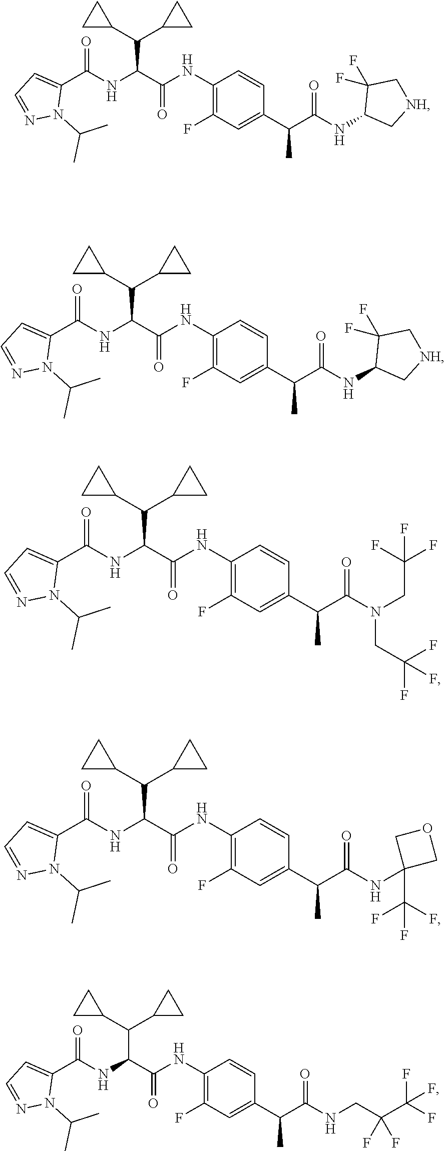 [SMILES: CC(C)n1nccc1C(=O)N[C@H](C(=O)Nc1ccc([C@H](C)C(=O)N[C@H]2CNCC2(F)F)cc1F)C(C1CC1)C1CC1 ; CC(C)n1nccc1C(=O)N[C@H](C(=O)Nc1ccc([C@H](C)C(=O)N[C@@H]2CNCC2(F)F)cc1F)C(C1CC1)C1CC1 ; CC(C)n1nccc1C(=O)N[C@H](C(=O)Nc1ccc([C@H](C)C(=O)N(CC(F)(F)F)CC(F)(F)F)cc1F)C(C1CC1)C1CC1 ; CC(C)n1nccc1C(=O)N[C@H](C(=O)Nc1ccc([C@H](C)C(=O)NC2(C(F)(F)F)COC2)cc1F)C(C1CC1)C1CC1 ; CC(C)n1nccc1C(=O)N[C@H](C(=O)Nc1ccc([C@H](C)C(=O)NCC(F)(F)C(F)(F)F)cc1F)C(C1CC1)C1CC1]

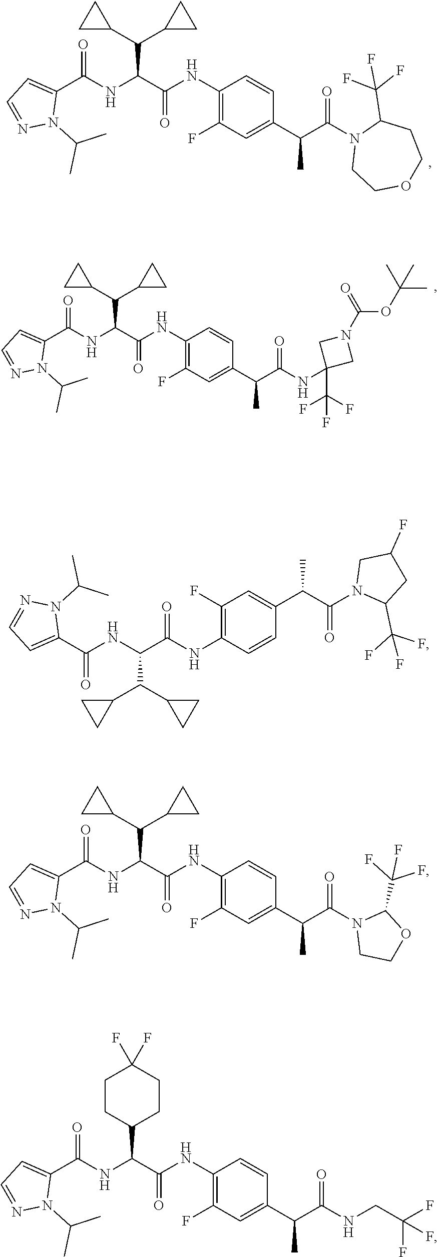 [SMILES: CC(C)n1nccc1C(=O)N[C@H](C(=O)Nc1ccc([C@H](C)C(=O)N2CCOCCC2C(F)(F)F)cc1F)C(C1CC1)C1CC1 ; CC(C)n1nccc1C(=O)N[C@H](C(=O)Nc1ccc([C@H](C)C(=O)NC2(C(F)(F)F)CN(C(=O)OC(C)(C)C)C2)cc1F)C(C1CC1)C1CC1 ; CC(C)n1nccc1C(=O)N[C@H](C(=O)Nc1ccc([C@H](C)C(=O)N2CC(F)CC2C(F)(F)F)cc1F)C(C1CC1)C1CC1 ; CC(C)n1nccc1C(=O)N[C@H](C(=O)Nc1ccc([C@H](C)C(=O)N2CCO[C@H]2C(F)(F)F)cc1F)C(C1CC1)C1CC1 ; CC(C)n1nccc1C(=O)N[C@H](C(=O)Nc1ccc([C@H](C)C(=O)NCC(F)(F)F)cc1F)C1CCC(F)(F)CC1]

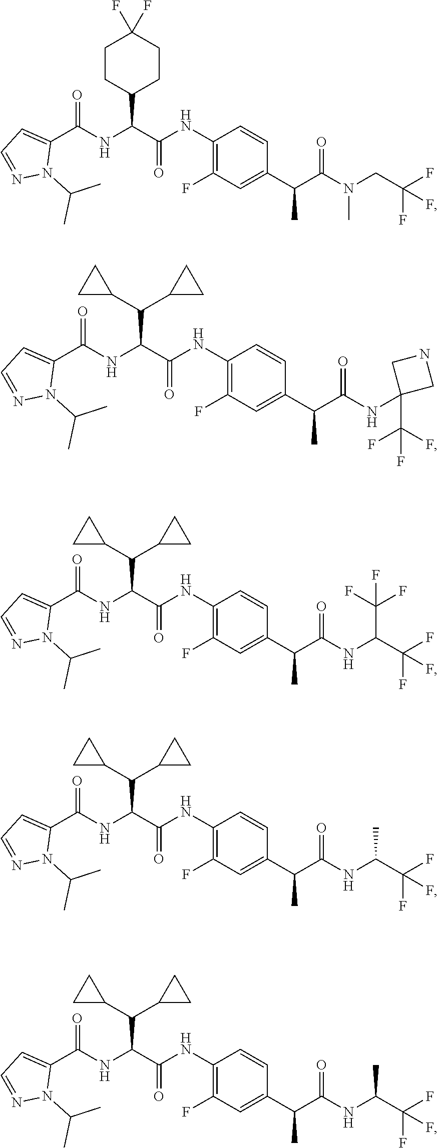 [SMILES: CC(C)n1nccc1C(=O)N[C@H](C(=O)Nc1ccc([C@H](C)C(=O)N(C)CC(F)(F)F)cc1F)C1CCC(F)(F)CC1 ; CC(C)n1nccc1C(=O)N[C@H](C(=O)Nc1ccc([C@H](C)C(=O)NC2(C(F)(F)F)CNC2)cc1F)C(C1CC1)C1CC1 ; CC(C)n1nccc1C(=O)N[C@H](C(=O)Nc1ccc([C@H](C)C(=O)NC(C(F)(F)F)C(F)(F)F)cc1F)C(C1CC1)C1CC1 ; CC(C)n1nccc1C(=O)N[C@H](C(=O)Nc1ccc([C@H](C)C(=O)N[C@H](C)C(F)(F)F)cc1F)C(C1CC1)C1CC1 ; CC(C)n1nccc1C(=O)N[C@H](C(=O)Nc1ccc([C@H](C)C(=O)N[C@@H](C)C(F)(F)F)cc1F)C(C1CC1)C1CC1]

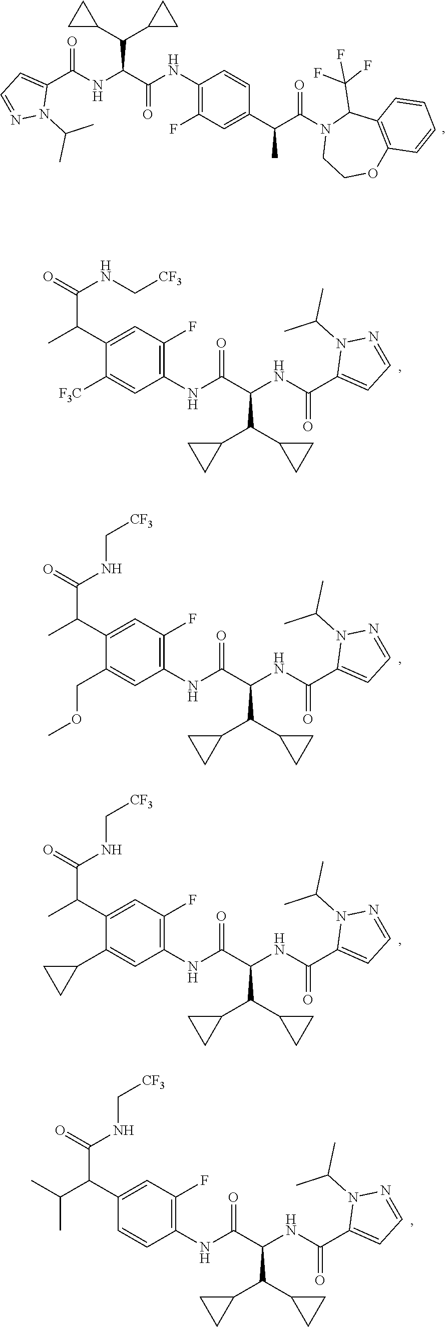 [SMILES: CC(C)n1nccc1C(=O)N[C@H](C(=O)Nc1ccc([C@H](C)C(=O)N2CCOc3ccccc3C2C(F)(F)F)cc1F)C(C1CC1)C1CC1 ; CC(C(=O)NCC(F)(F)F)c1cc(F)c(NC(=O)[C@@H](NC(=O)c2ccnn2C(C)C)C(C2CC2)C2CC2)cc1C(F)(F)F ; COCc1cc(NC(=O)[C@@H](NC(=O)c2ccnn2C(C)C)C(C2CC2)C2CC2)c(F)cc1C(C)C(=O)NCC(F)(F)F ; CC(C(=O)NCC(F)(F)F)c1cc(F)c(NC(=O)[C@@H](NC(=O)c2ccnn2C(C)C)C(C2CC2)C2CC2)cc1C1CC1 ; CC(C)C(C(=O)NCC(F)(F)F)c1ccc(NC(=O)[C@@H](NC(=O)c2ccnn2C(C)C)C(C2CC2)C2CC2)c(F)c1]

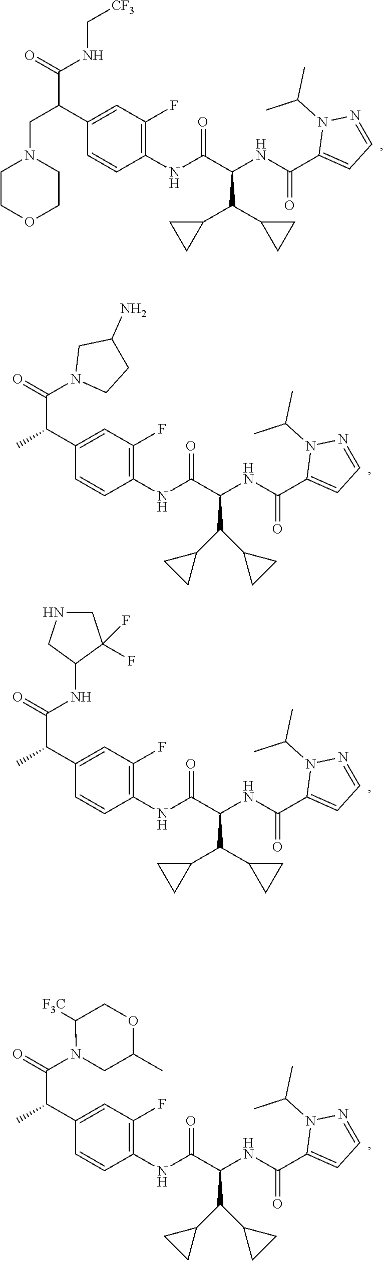 [SMILES: CC(C)n1nccc1C(=O)N[C@H](C(=O)Nc1ccc(C(CN2CCOCC2)C(=O)NCC(F)(F)F)cc1F)C(C1CC1)C1CC1 ; CC(C)n1nccc1C(=O)N[C@H](C(=O)Nc1ccc([C@H](C)C(=O)N2CCC(N)C2)cc1F)C(C1CC1)C1CC1 ; CC(C)n1nccc1C(=O)N[C@H](C(=O)Nc1ccc([C@H](C)C(=O)NC2CNCC2(F)F)cc1F)C(C1CC1)C1CC1 ; CC1CN(C(=O)[C@@H](C)c2ccc(NC(=O)[C@@H](NC(=O)c3ccnn3C(C)C)C(C3CC3)C3CC3)c(F)c2)C(C(F)(F)F)CO1]

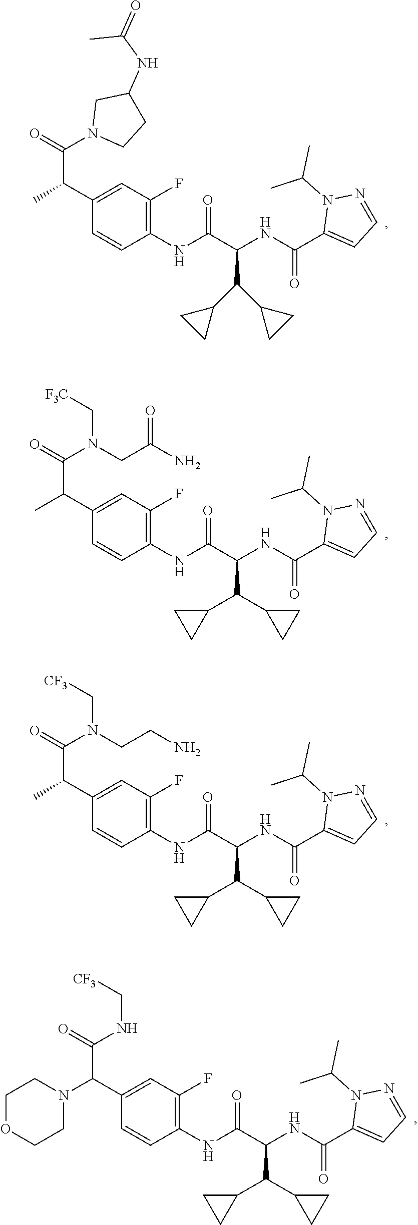 [SMILES: CC(=O)NC1CCN(C(=O)[C@@H](C)c2ccc(NC(=O)[C@@H](NC(=O)c3ccnn3C(C)C)C(C3CC3)C3CC3)c(F)c2)C1 ; CC(C(=O)N(CC(N)=O)CC(F)(F)F)c1ccc(NC(=O)[C@@H](NC(=O)c2ccnn2C(C)C)C(C2CC2)C2CC2)c(F)c1 ; CC(C)n1nccc1C(=O)N[C@H](C(=O)Nc1ccc([C@H](C)C(=O)N(CCN)CC(F)(F)F)cc1F)C(C1CC1)C1CC1 ; CC(C)n1nccc1C(=O)N[C@H](C(=O)Nc1ccc(C(C(=O)NCC(F)(F)F)N2CCOCC2)cc1F)C(C1CC1)C1CC1]

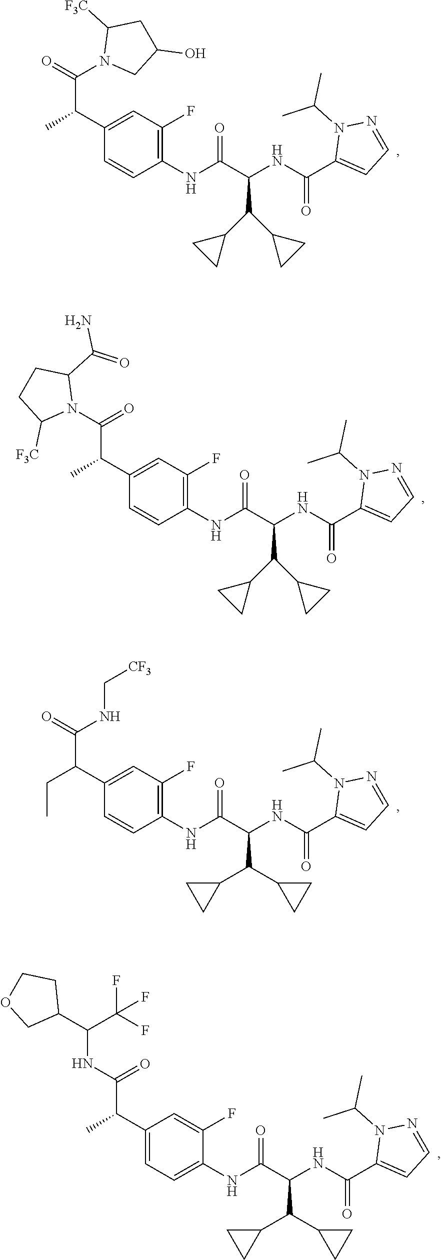 [SMILES: CC(C)n1nccc1C(=O)N[C@H](C(=O)Nc1ccc([C@H](C)C(=O)N2CC(O)CC2C(F)(F)F)cc1F)C(C1CC1)C1CC1 ; CC(C)n1nccc1C(=O)N[C@H](C(=O)Nc1ccc([C@H](C)C(=O)N2C(C(N)=O)CCC2C(F)(F)F)cc1F)C(C1CC1)C1CC1 ; CCC(C(=O)NCC(F)(F)F)c1ccc(NC(=O)[C@@H](NC(=O)c2ccnn2C(C)C)C(C2CC2)C2CC2)c(F)c1 ; CC(C)n1nccc1C(=O)N[C@H](C(=O)Nc1ccc([C@H](C)C(=O)NC(C2CCOC2)C(F)(F)F)cc1F)C(C1CC1)C1CC1]

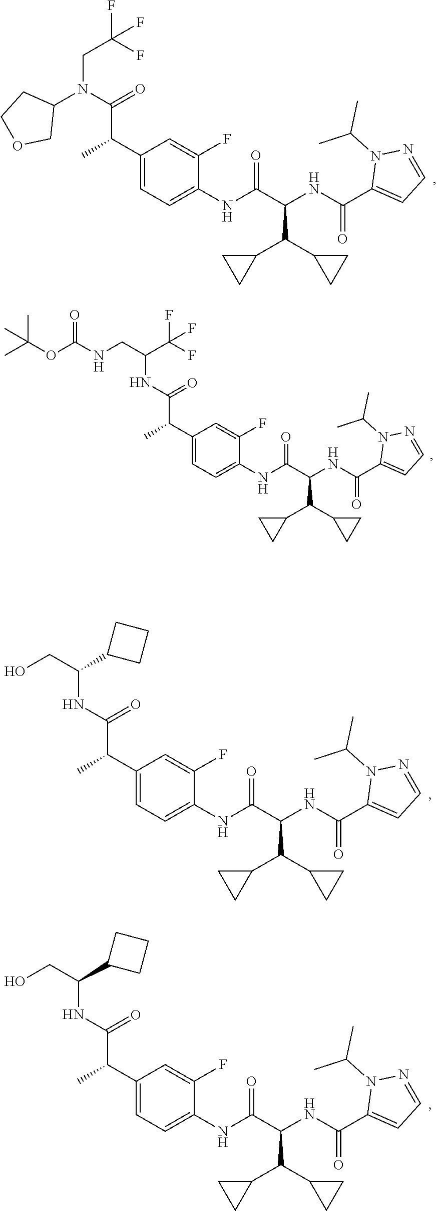 [SMILES: CC(C)n1nccc1C(=O)N[C@H](C(=O)Nc1ccc([C@H](C)C(=O)N(CC(F)(F)F)C2CCOC2)cc1F)C(C1CC1)C1CC1 ; CC(C)n1nccc1C(=O)N[C@H](C(=O)Nc1ccc([C@H](C)C(=O)NC(CNC(=O)OC(C)(C)C)C(F)(F)F)cc1F)C(C1CC1)C1CC1 ; CC(C)n1nccc1C(=O)N[C@H](C(=O)Nc1ccc([C@H](C)C(=O)N[C@H](CO)C2CCC2)cc1F)C(C1CC1)C1CC1 ; CC(C)n1nccc1C(=O)N[C@H](C(=O)Nc1ccc([C@H](C)C(=O)N[C@@H](CO)C2CCC2)cc1F)C(C1CC1)C1CC1]

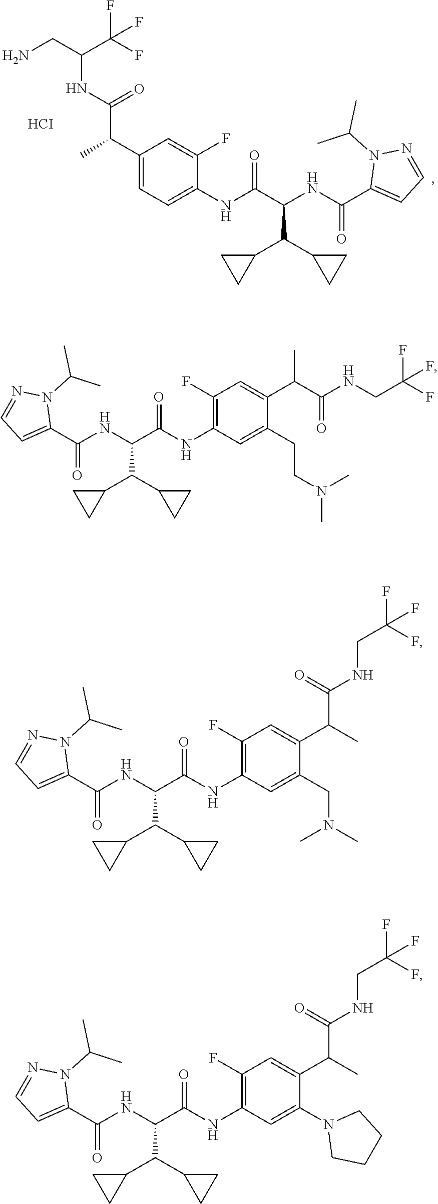 [SMILES: "HCI" ; CC(C)n1nccc1C(=O)N[C@H](C(=O)Nc1ccc([C@H](C)C(=O)NC(CN)C(F)(F)F)cc1F)C(C1CC1)C1CC1 ; CC(C(=O)NCC(F)(F)F)c1cc(F)c(NC(=O)[C@@H](NC(=O)c2ccnn2C(C)C)C(C2CC2)C2CC2)cc1CCN(C)C ; CC(C(=O)NCC(F)(F)F)c1cc(F)c(NC(=O)[C@@H](NC(=O)c2ccnn2C(C)C)C(C2CC2)C2CC2)cc1CN(C)C ; CC(C(=O)NCC(F)(F)F)c1cc(F)c(NC(=O)[C@@H](NC(=O)c2ccnn2C(C)C)C(C2CC2)C2CC2)cc1N1CCCC1]

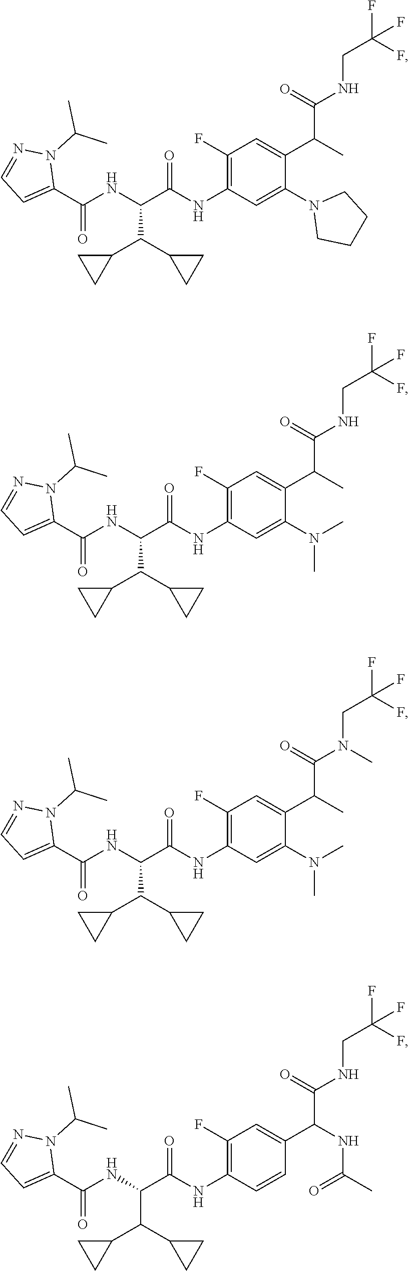 [SMILES: CC(C(=O)NCC(F)(F)F)c1cc(F)c(NC(=O)[C@@H](NC(=O)c2ccnn2C(C)C)C(C2CC2)C2CC2)cc1N1CCCC1 ; CC(C(=O)NCC(F)(F)F)c1cc(F)c(NC(=O)[C@@H](NC(=O)c2ccnn2C(C)C)C(C2CC2)C2CC2)cc1N(C)C ; CC(C(=O)N(C)CC(F)(F)F)c1cc(F)c(NC(=O)[C@@H](NC(=O)c2ccnn2C(C)C)C(C2CC2)C2CC2)cc1N(C)C ; CC(=O)NC(C(=O)NCC(F)(F)F)c1ccc(NC(=O)[C@@H](NC(=O)c2ccnn2C(C)C)C(C2CC2)C2CC2)c(F)c1]

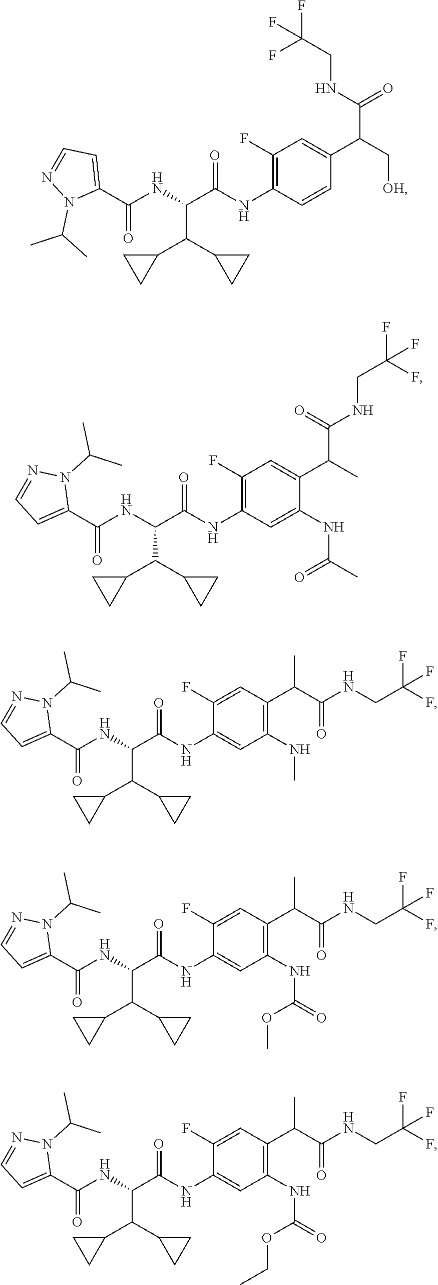 [SMILES: CC(C)n1nccc1C(=O)N[C@H](C(=O)Nc1ccc(C(CO)C(=O)NCC(F)(F)F)cc1F)C(C1CC1)C1CC1 ; CC(=O)Nc1cc(NC(=O)[C@@H](NC(=O)c2ccnn2C(C)C)C(C2CC2)C2CC2)c(F)cc1C(C)C(=O)NCC(F)(F)F ; CNc1cc(NC(=O)[C@@H](NC(=O)c2ccnn2C(C)C)C(C2CC2)C2CC2)c(F)cc1C(C)C(=O)NCC(F)(F)F ; COC(=O)Nc1cc(NC(=O)[C@@H](NC(=O)c2ccnn2C(C)C)C(C2CC2)C2CC2)c(F)cc1C(C)C(=O)NCC(F)(F)F ; CCOC(=O)Nc1cc(NC(=O)[C@@H](NC(=O)c2ccnn2C(C)C)C(C2CC2)C2CC2)c(F)cc1C(C)C(=O)NCC(F)(F)F]

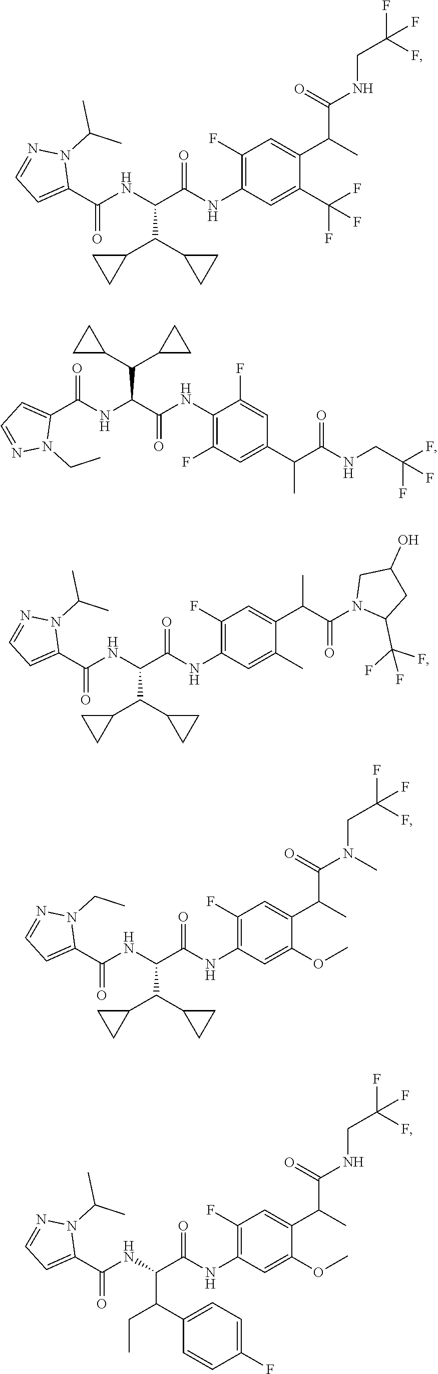 [SMILES: CC(C(=O)NCC(F)(F)F)c1cc(F)c(NC(=O)[C@@H](NC(=O)c2ccnn2C(C)C)C(C2CC2)C2CC2)cc1C(F)(F)F ; CCn1nccc1C(=O)N[C@H](C(=O)Nc1c(F)cc(C(C)C(=O)NCC(F)(F)F)cc1F)C(C1CC1)C1CC1 ; Cc1cc(NC(=O)[C@@H](NC(=O)c2ccnn2C(C)C)C(C2CC2)C2CC2)c(F)cc1C(C)C(=O)N1CC(O)CC1C(F)(F)F ; CCn1nccc1C(=O)N[C@H](C(=O)Nc1cc(OC)c(C(C)C(=O)N(C)CC(F)(F)F)cc1F)C(C1CC1)C1CC1 ; CCC(c1ccc(F)cc1)[C@H](NC(=O)c1ccnn1C(C)C)C(=O)Nc1cc(OC)c(C(C)C(=O)NCC(F)(F)F)cc1F]

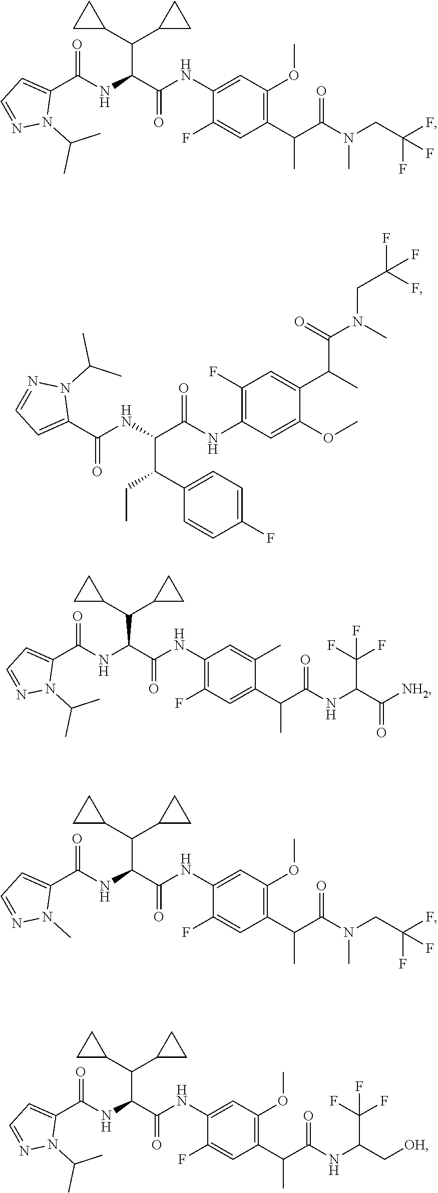 [SMILES: COc1cc(NC(=O)[C@@H](NC(=O)c2ccnn2C(C)C)C(C2CC2)C2CC2)c(F)cc1C(C)C(=O)N(C)CC(F)(F)F ; CC[C@@H](c1ccc(F)cc1)[C@H](NC(=O)c1ccnn1C(C)C)C(=O)Nc1cc(OC)c(C(C)C(=O)N(C)CC(F)(F)F)cc1F ; Cc1cc(NC(=O)[C@@H](NC(=O)c2ccnn2C(C)C)C(C2CC2)C2CC2)c(F)cc1C(C)C(=O)NC(C(N)=O)C(F)(F)F ; COc1cc(NC(=O)[C@@H](NC(=O)c2ccnn2C)C(C2CC2)C2CC2)c(F)cc1C(C)C(=O)N(C)CC(F)(F)F ; COc1cc(NC(=O)[C@@H](NC(=O)c2ccnn2C(C)C)C(C2CC2)C2CC2)c(F)cc1C(C)C(=O)NC(CO)C(F)(F)F]

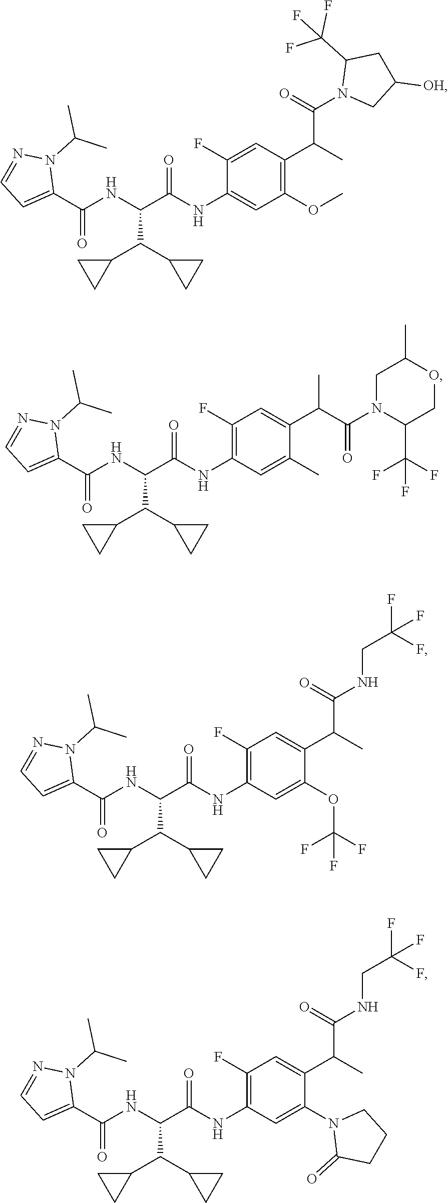 [SMILES: COc1cc(NC(=O)[C@@H](NC(=O)c2ccnn2C(C)C)C(C2CC2)C2CC2)c(F)cc1C(C)C(=O)N1CC(O)CC1C(F)(F)F ; Cc1cc(NC(=O)[C@@H](NC(=O)c2ccnn2C(C)C)C(C2CC2)C2CC2)c(F)cc1C(C)C(=O)N1CC(C)OCC1C(F)(F)F ; CC(C(=O)NCC(F)(F)F)c1cc(F)c(NC(=O)[C@@H](NC(=O)c2ccnn2C(C)C)C(C2CC2)C2CC2)cc1OC(F)(F)F ; CC(C(=O)NCC(F)(F)F)c1cc(F)c(NC(=O)[C@@H](NC(=O)c2ccnn2C(C)C)C(C2CC2)C2CC2)cc1N1CCCC1=O]

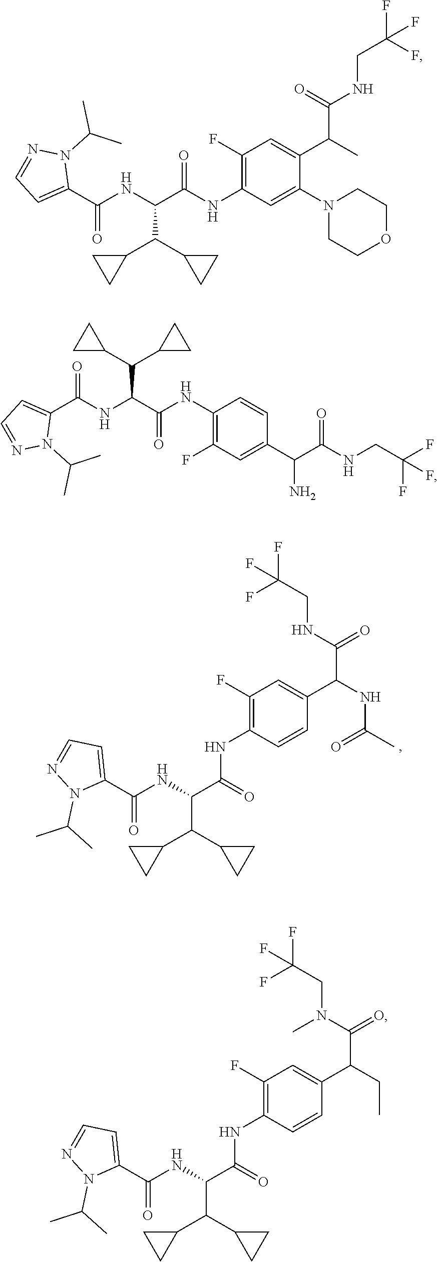

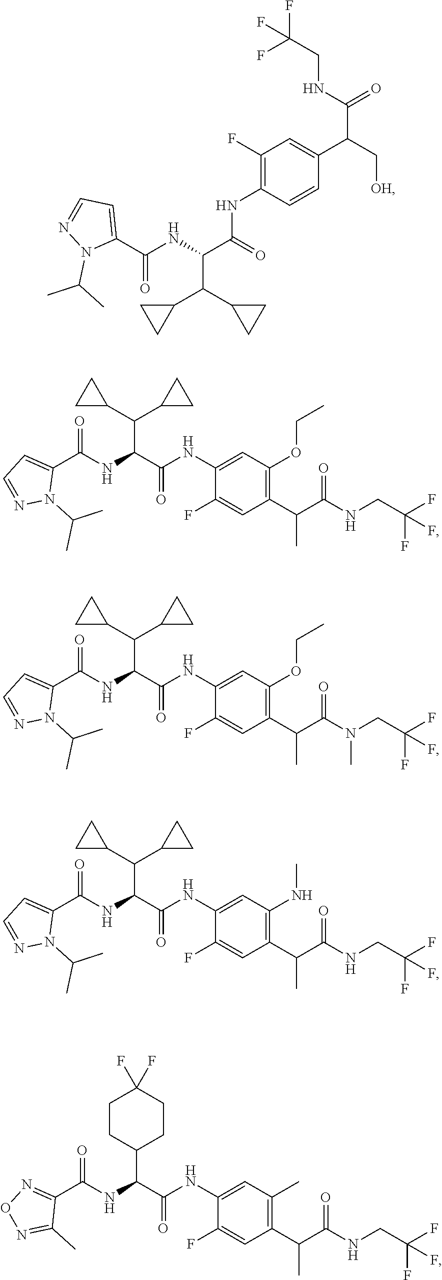 [SMILES: CC(C)n1nccc1C(=O)N[C@H](C(=O)Nc1ccc(C(CO)C(=O)NCC(F)(F)F)cc1F)C(C1CC1)C1CC1 ; CCOc1cc(NC(=O)[C@@H](NC(=O)c2ccnn2C(C)C)C(C2CC2)C2CC2)c(F)cc1C(C)C(=O)NCC(F)(F)F ; CCOc1cc(NC(=O)[C@@H](NC(=O)c2ccnn2C(C)C)C(C2CC2)C2CC2)c(F)cc1C(C)C(=O)N(C)CC(F)(F)F ; CNc1cc(NC(=O)[C@@H](NC(=O)c2ccnn2C(C)C)C(C2CC2)C2CC2)c(F)cc1C(C)C(=O)NCC(F)(F)F ; Cc1cc(NC(=O)[C@@H](NC(=O)c2nonc2C)C2CCC(F)(F)CC2)c(F)cc1C(C)C(=O)NCC(F)(F)F]

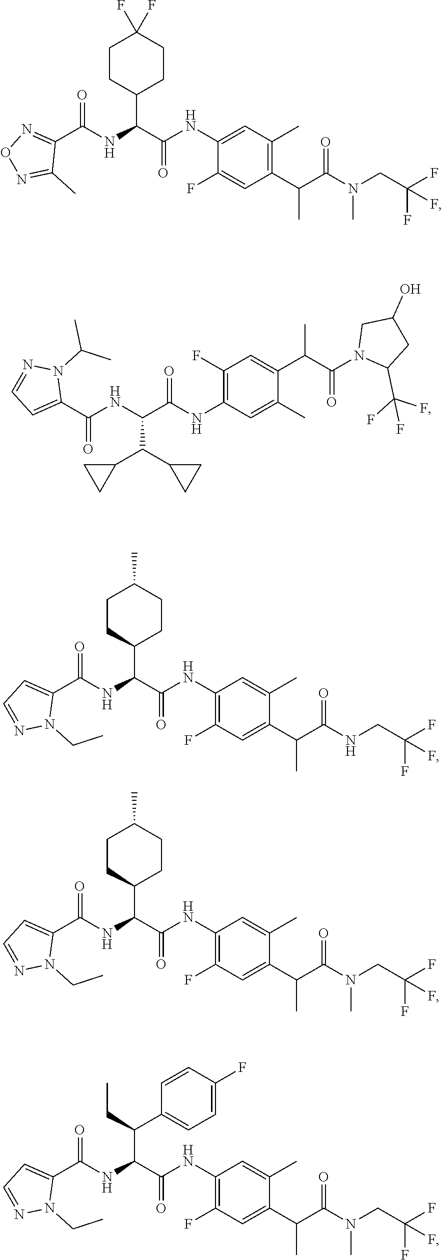 [SMILES: Cc1cc(NC(=O)[C@@H](NC(=O)c2nonc2C)C2CCC(F)(F)CC2)c(F)cc1C(C)C(=O)N(C)CC(F)(F)F ; Cc1cc(NC(=O)[C@@H](NC(=O)c2ccnn2C(C)C)C(C2CC2)C2CC2)c(F)cc1C(C)C(=O)N1CC(O)CC1C(F)(F)F ; CCn1nccc1C(=O)N[C@H](C(=O)Nc1cc(C)c(C(C)C(=O)NCC(F)(F)F)cc1F)[C@H]1CC[C@H](C)CC1 ; CCn1nccc1C(=O)N[C@H](C(=O)Nc1cc(C)c(C(C)C(=O)N(C)CC(F)(F)F)cc1F)[C@H]1CC[C@H](C)CC1 ; CC[C@@H](c1ccc(F)cc1)[C@H](NC(=O)c1ccnn1CC)C(=O)Nc1cc(C)c(C(C)C(=O)N(C)CC(F)(F)F)cc1F]

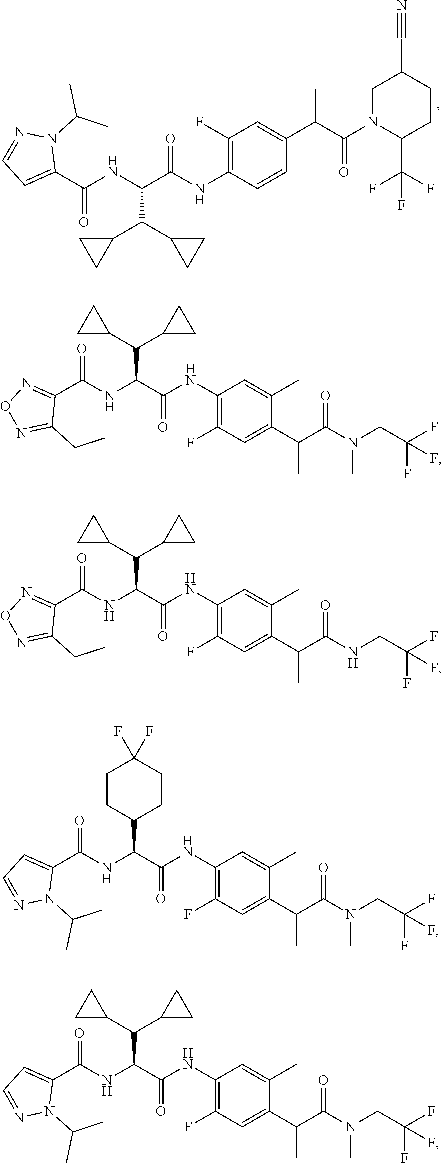

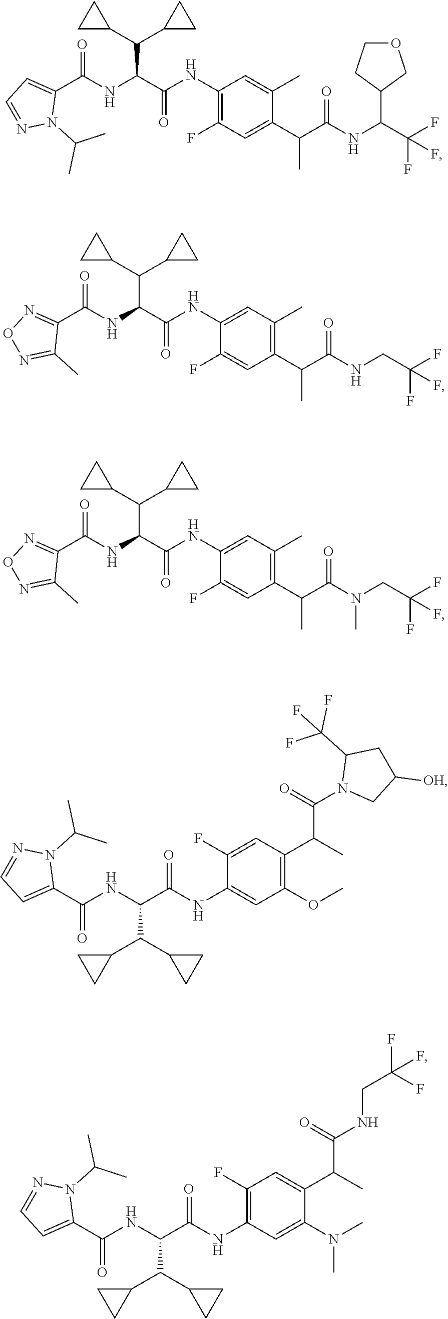 [SMILES: Cc1cc(NC(=O)[C@@H](NC(=O)c2ccnn2C(C)C)C(C2CC2)C2CC2)c(F)cc1C(C)C(=O)NC(C1CCOC1)C(F)(F)F ; Cc1cc(NC(=O)[C@@H](NC(=O)c2nonc2C)C(C2CC2)C2CC2)c(F)cc1C(C)C(=O)NCC(F)(F)F ; Cc1cc(NC(=O)[C@@H](NC(=O)c2nonc2C)C(C2CC2)C2CC2)c(F)cc1C(C)C(=O)N(C)CC(F)(F)F ; COc1cc(NC(=O)[C@@H](NC(=O)c2ccnn2C(C)C)C(C2CC2)C2CC2)c(F)cc1C(C)C(=O)N1CC(O)CC1C(F)(F)F ; CC(C(=O)NCC(F)(F)F)c1cc(F)c(NC(=O)[C@@H](NC(=O)c2ccnn2C(C)C)C(C2CC2)C2CC2)cc1N(C)C]

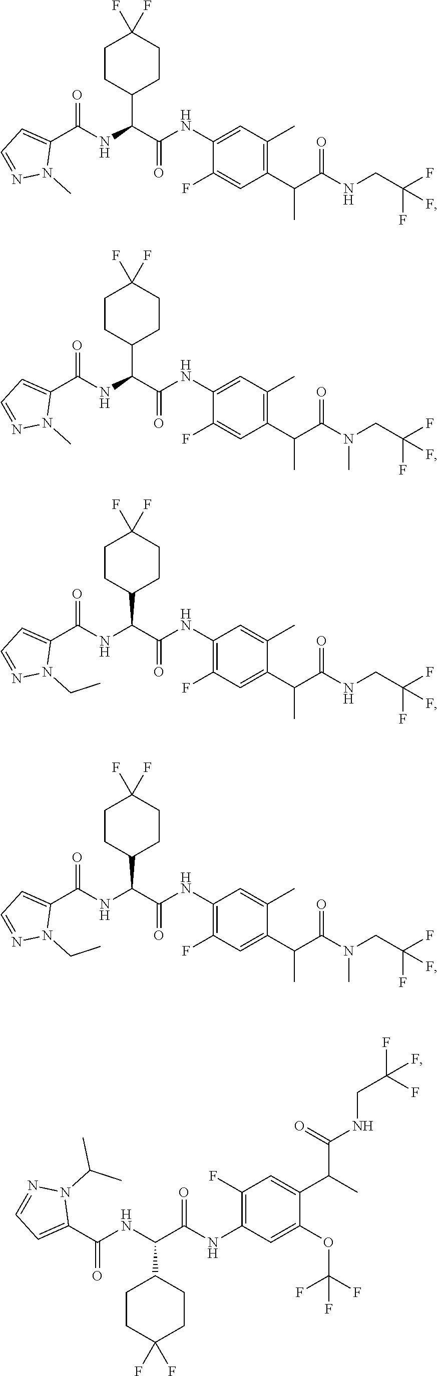 [SMILES: Cc1cc(NC(=O)[C@@H](NC(=O)c2ccnn2C)C2CCC(F)(F)CC2)c(F)cc1C(C)C(=O)NCC(F)(F)F ; Cc1cc(NC(=O)[C@@H](NC(=O)c2ccnn2C)C2CCC(F)(F)CC2)c(F)cc1C(C)C(=O)N(C)CC(F)(F)F ; CCn1nccc1C(=O)N[C@H](C(=O)Nc1cc(C)c(C(C)C(=O)NCC(F)(F)F)cc1F)C1CCC(F)(F)CC1 ; CCn1nccc1C(=O)N[C@H](C(=O)Nc1cc(C)c(C(C)C(=O)N(C)CC(F)(F)F)cc1F)C1CCC(F)(F)CC1 ; CC(C(=O)NCC(F)(F)F)c1cc(F)c(NC(=O)[C@@H](NC(=O)c2ccnn2C(C)C)C2CCC(F)(F)CC2)cc1OC(F)(F)F]

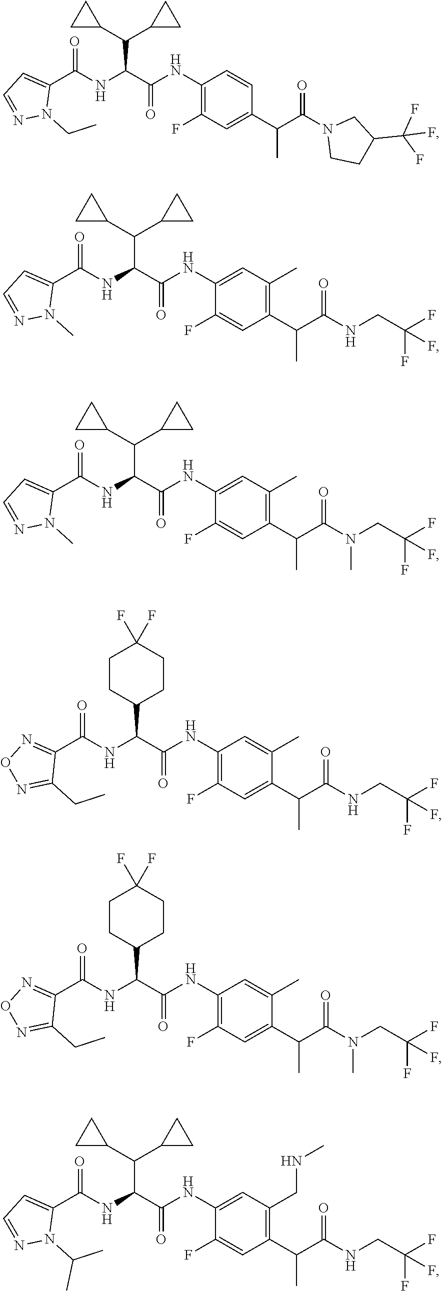 [SMILES: CCn1nccc1C(=O)N[C@H](C(=O)Nc1ccc(C(C)C(=O)N2CCC(C(F)(F)F)C2)cc1F)C(C1CC1)C1CC1 ; Cc1cc(NC(=O)[C@@H](NC(=O)c2ccnn2C)C(C2CC2)C2CC2)c(F)cc1C(C)C(=O)NCC(F)(F)F ; Cc1cc(NC(=O)[C@@H](NC(=O)c2ccnn2C)C(C2CC2)C2CC2)c(F)cc1C(C)C(=O)N(C)CC(F)(F)F ; CCc1nonc1C(=O)N[C@H](C(=O)Nc1cc(C)c(C(C)C(=O)NCC(F)(F)F)cc1F)C1CCC(F)(F)CC1 ; CCc1nonc1C(=O)N[C@H](C(=O)Nc1cc(C)c(C(C)C(=O)N(C)CC(F)(F)F)cc1F)C1CCC(F)(F)CC1 ; CNCc1cc(NC(=O)[C@@H](NC(=O)c2ccnn2C(C)C)C(C2CC2)C2CC2)c(F)cc1C(C)C(=O)NCC(F)(F)F]

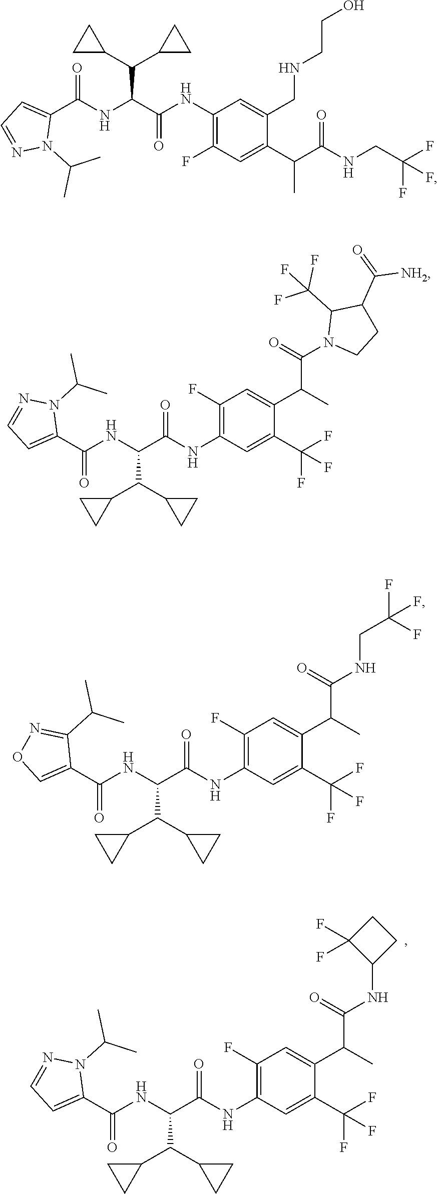 [SMILES: CC(C(=O)NCC(F)(F)F)c1cc(F)c(NC(=O)[C@@H](NC(=O)c2ccnn2C(C)C)C(C2CC2)C2CC2)cc1CNCCO ; CC(C(=O)N1CCC(C(N)=O)C1C(F)(F)F)c1cc(F)c(NC(=O)[C@@H](NC(=O)c2ccnn2C(C)C)C(C2CC2)C2CC2)cc1C(F)(F)F ; CC(C)c1nocc1C(=O)N[C@H](C(=O)Nc1cc(C(F)(F)F)c(C(C)C(=O)NCC(F)(F)F)cc1F)C(C1CC1)C1CC1 ; CC(C(=O)NC1CCC1(F)F)c1cc(F)c(NC(=O)[C@@H](NC(=O)c2ccnn2C(C)C)C(C2CC2)C2CC2)cc1C(F)(F)F]

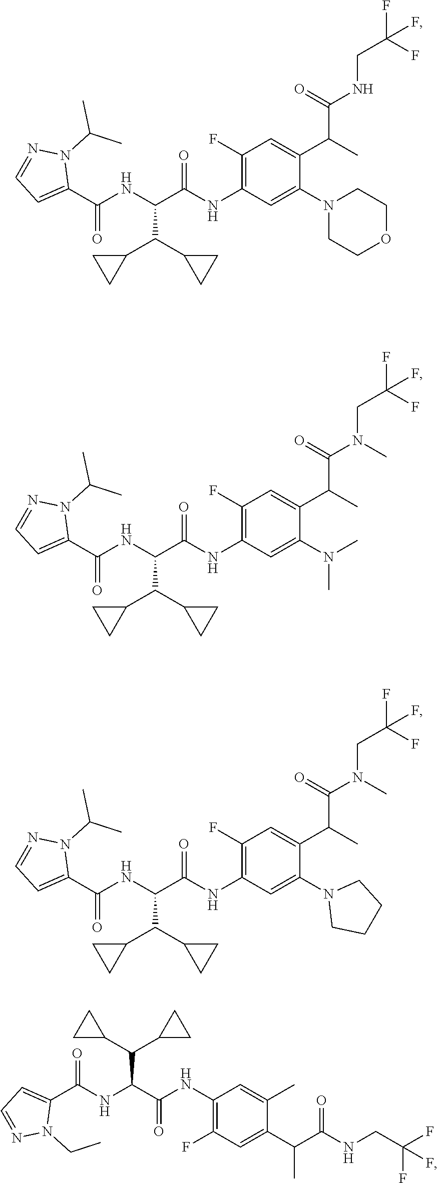

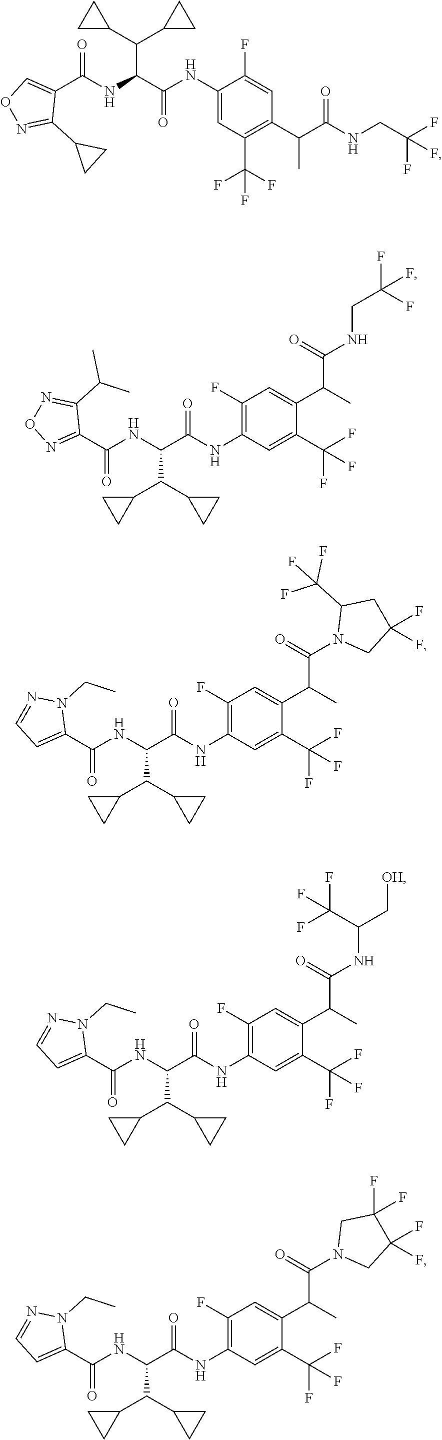 [SMILES: CC(C(=O)NCC(F)(F)F)c1cc(F)c(NC(=O)[C@@H](NC(=O)c2conc2C2CC2)C(C2CC2)C2CC2)cc1C(F)(F)F ; CC(C)c1nonc1C(=O)N[C@H](C(=O)Nc1cc(C(F)(F)F)c(C(C)C(=O)NCC(F)(F)F)cc1F)C(C1CC1)C1CC1 ; CCn1nccc1C(=O)N[C@H](C(=O)Nc1cc(C(F)(F)F)c(C(C)C(=O)N2CC(F)(F)CC2C(F)(F)F)cc1F)C(C1CC1)C1CC1 ; CCn1nccc1C(=O)N[C@H](C(=O)Nc1cc(C(F)(F)F)c(C(C)C(=O)NC(CO)C(F)(F)F)cc1F)C(C1CC1)C1CC1 ; CCn1nccc1C(=O)N[C@H](C(=O)Nc1cc(C(F)(F)F)c(C(C)C(=O)N2CC(F)(F)C(F)(F)C2)cc1F)C(C1CC1)C1CC1]

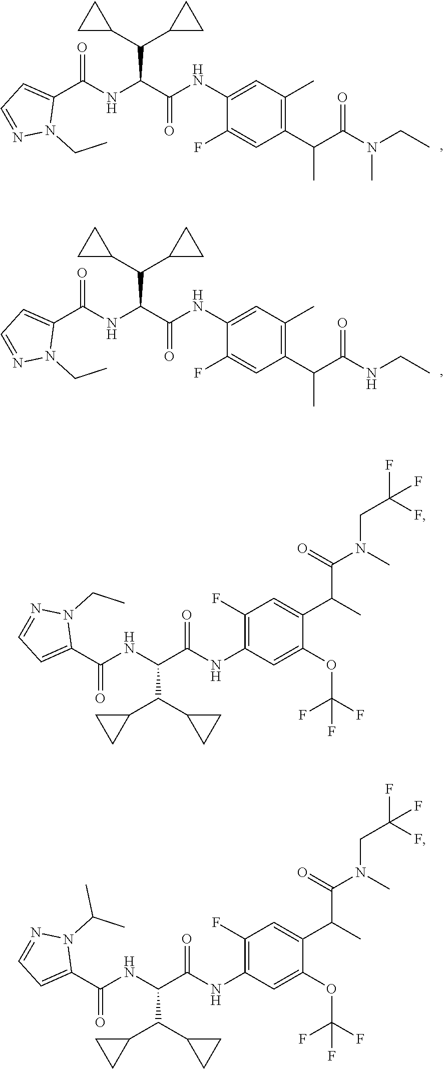 [SMILES: CCN(C)C(=O)C(C)c1cc(F)c(NC(=O)[C@@H](NC(=O)c2ccnn2CC)C(C2CC2)C2CC2)cc1C ; CCNC(=O)C(C)c1cc(F)c(NC(=O)[C@@H](NC(=O)c2ccnn2CC)C(C2CC2)C2CC2)cc1C ; CCn1nccc1C(=O)N[C@H](C(=O)Nc1cc(OC(F)(F)F)c(C(C)C(=O)N(C)CC(F)(F)F)cc1F)C(C1CC1)C1CC1 ; CC(C(=O)N(C)CC(F)(F)F)c1cc(F)c(NC(=O)[C@@H](NC(=O)c2ccnn2C(C)C)C(C2CC2)C2CC2)cc1OC(F)(F)F]

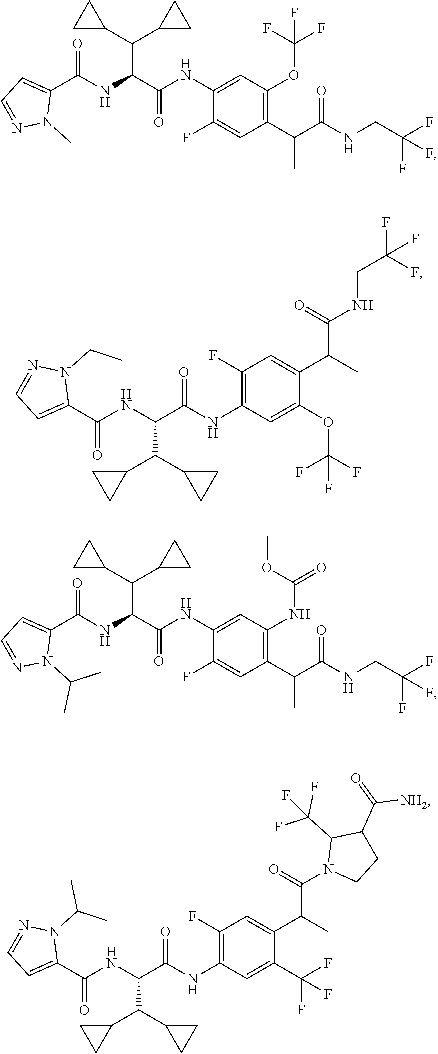 [SMILES: CC(C(=O)NCC(F)(F)F)c1cc(F)c(NC(=O)[C@@H](NC(=O)c2ccnn2C)C(C2CC2)C2CC2)cc1OC(F)(F)F ; CCn1nccc1C(=O)N[C@H](C(=O)Nc1cc(OC(F)(F)F)c(C(C)C(=O)NCC(F)(F)F)cc1F)C(C1CC1)C1CC1 ; COC(=O)Nc1cc(NC(=O)[C@@H](NC(=O)c2ccnn2C(C)C)C(C2CC2)C2CC2)c(F)cc1C(C)C(=O)NCC(F)(F)F ; CC(C(=O)N1CCC(C(N)=O)C1C(F)(F)F)c1cc(F)c(NC(=O)[C@@H](NC(=O)c2ccnn2C(C)C)C(C2CC2)C2CC2)cc1C(F)(F)F]

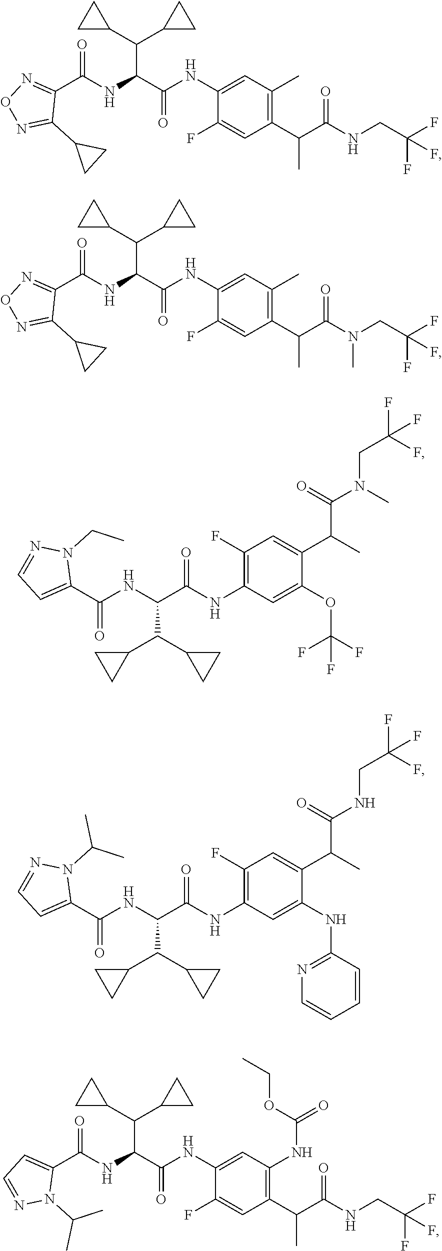

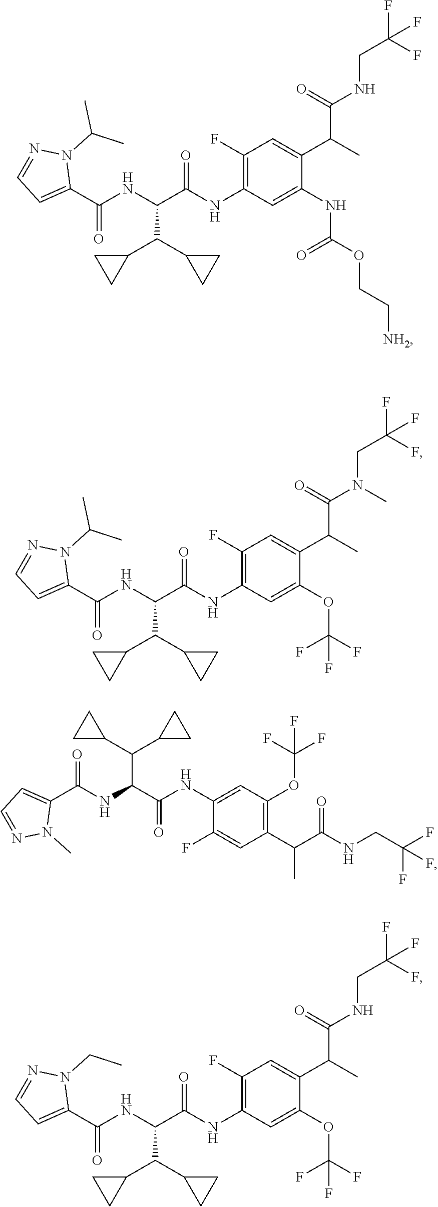

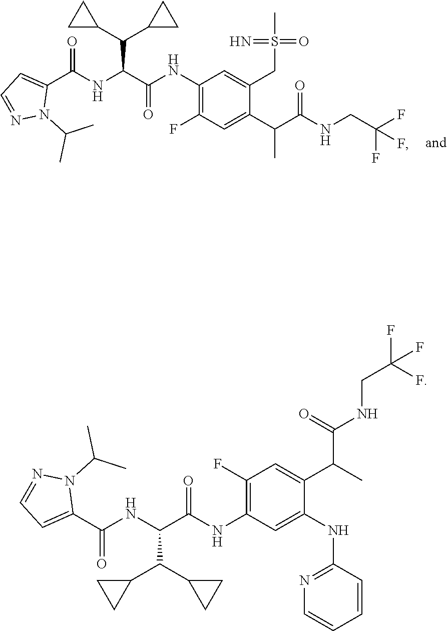 [SMILES: CC(C(=O)NCC(F)(F)F)c1cc(F)c(NC(=O)[C@@H](NC(=O)c2ccnn2C(C)C)C(C2CC2)C2CC2)cc1CS(C)(=N)=O ; CC(C(=O)NCC(F)(F)F)c1cc(F)c(NC(=O)[C@@H](NC(=O)c2ccnn2C(C)C)C(C2CC2)C2CC2)cc1Nc1ccccn1]

or a salt of any one thereof.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

Chemical entities having carbon-carbon double bonds or carbon-nitrogen double bonds may exist in Z—or F—form (or cis- or trans- form). Furthermore, some chemical entities may exist in various tautomeric forms. Unless otherwise specified, compounds or salts of Formula (I), (I-a), or (II), are intended to include all Z—, E- and tautomeric forms as well.

“Isomers” are different compounds that have the same molecular formula. “Stereoisomers” are isomers that differ only in the way the atoms are arranged in space. “Enantiomers” are a pair of stereoisomers that are non-superimposable mirror images of each other. A 1:1 mixture of a pair of enantiomers is a “racemic” mixture. The term “(t)” is used to designate a racemic mixture where appropriate. “Diastereoisomers” or “diastereomers” are stereoisomers that have at least two asymmetric atoms but are not mirror images of each other. The absolute stereochemistry is specified according to the Cahn-Ingold-Prelog R—S system. When a compound is a pure enantiomer, the stereochemistry at each chiral carbon can be specified by either R or S. Resolved compounds whose absolute configuration is unknown can be designated (+) or (−) depending on the direction (dextro- or levorotatory) in which they rotate plane polarized light at the wavelength of the sodium D line. Certain compounds described herein contain one or more asymmetric centers and can thus give rise to enantiomers, diastereomers, and other stereoisomeric forms, the asymmetric centers of which can be defined, in terms of absolute stereochemistry, as (R)- or (S)-. The present chemical entities, pharmaceutical compositions and methods are meant to include all such possible stereoisomers, including racemic mixtures, optically pure forms, mixtures of diastereomers and intermediate mixtures. Optically active (R)- and (S)-isomers can be prepared using chiral synthons or chiral reagents, or resolved using conventional techniques. The optical activity of a compound can be analyzed via any suitable method, including but not limited to chiral chromatography and polarimetry, and the degree of predominance of one stereoisomer over the other isomer can be determined.

The compounds or salts for Formula (I), (I-a), or (II), may in some cases exist as diastereomers, enantiomers, or other stereoisomeric forms. The compounds presented herein include all diastereomeric, enantiomeric, and epimeric forms as well as the racemates, mixtures of diastereomers, and other mixtures thereof, to the extent they can be made by one of ordinary skill in the art by routine experimentation. Separation of stereoisomers may be performed by chromatography or by forming diastereomers and separating by recrystallization, or chromatography, or any combination thereof. (Jean Jacques, Andre Collet, Samuel H. Wilen, “Enantiomers, Racemates and Resolutions”, John Wiley And Sons, Inc, 1981, herein incorporated by reference for this disclosure). Stereoisomers may also be obtained by stereoselective synthesis. Furthermore, a mixture of two enantiomers enriched in one of the two can be purified to provide further optically enriched form of the major enantiomer by recrystallization and/or trituration.

In certain embodiments, compounds or salts for Formula (I), (I-a), or (II), may comprise two or more enantiomers or diastereomers of a compound wherein a single enantiomer or diastereomer accounts for at least about 70% by weight, at least about 80% by weight, at least about 90% by weight, at least about 98% by weight, or at least about 99% by weight or more of the total weight of all stereoisomers. Methods of producing substantially pure enantiomers are well known to those of skill in the art. For example, a single stereoisomer, e.g, an enantiomer, substantially free of its stereoisomer may be obtained by resolution of the racemic mixture using a method such as formation of diastereomers using optically active resolving agents (Stereochemistry of Carbon Compounds, (1962) by E. L. Eliel, McGraw Hill: Lochmuller (1975) J. Chromatogr, 113(3): 283-302). Racemic mixtures of chiral compounds can be separated and isolated by any suitable method, including, but not limited to: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. Another approach for separation of the enantiomers is to use a Diacel chiral column and elution using an organic mobile phase such as done by Chiral Technologies (www.chiraltech.com) on a fee for service basis.

A “tautomer” refers to a molecule wherein a proton shift from one atom of a molecule to another atom of the same molecule is possible. In certain embodiments, the compounds or salts for Formula (I), (I-a), or (II), exist as tautomers. In circumstances where tautomerization is possible, a chemical equilibrium of the tautomers may exist. The exact ratio of the tautomers depends on several factors, including physical state, temperature, solvent, and pH. Some non-limiting examples of tautomeric equilibrium include:

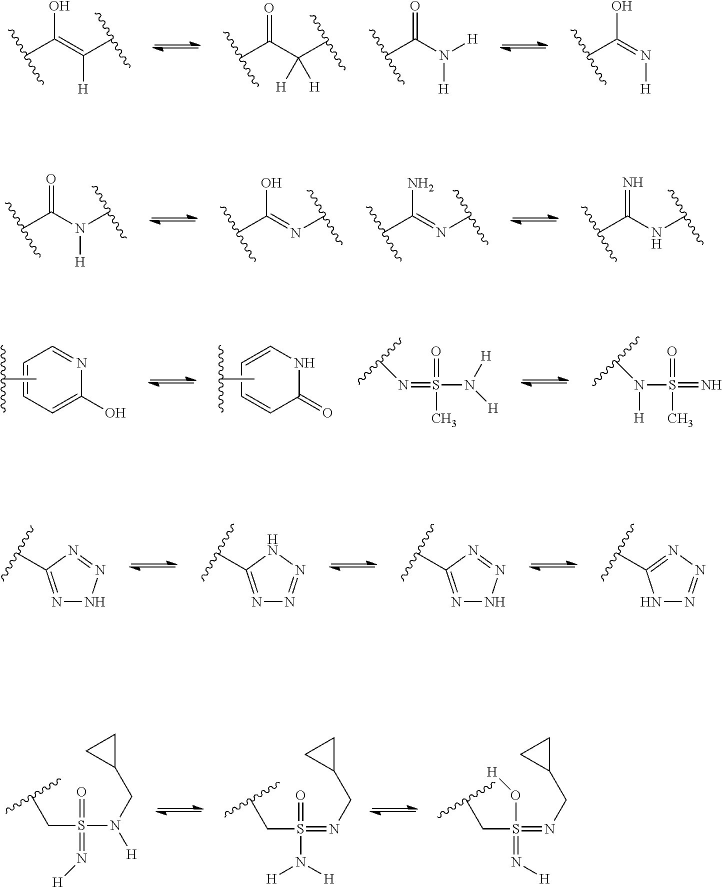

The compounds of Formula (I), (I-a), or (II), can be used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

In certain embodiments, the compounds of Formula (I), (I-a), or (II), have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [In: Curr, Pharm. Des, 2000: 6(10)] 2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem. 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Unless otherwise stated, compounds of Formula (I), (I-a), or (II), are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of Formula (I), (I-a), or (II), optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C, ¹⁴C, ⁵C, ¹²N, ¹³N, ¹⁵N, ¹⁶N, ¹⁶O, ¹⁶O, ¹⁴F, ¹⁵F, ¹⁶F, ¹⁷F, ¹⁸F, ³³, ³⁴S, ³⁵S, ³⁶S, ²⁵C. ³⁷Cl, ⁷⁹Br, ⁸¹Br, and ¹²⁵I are all contemplated. All isotopic variations of the compounds of the present invention, whether radioactive or not, are encompassed within the scope of the present invention.

Included in the present disclosure are salts, particularly pharmaceutically acceptable salts, of the compounds of Formula (I), (I-a), or (II). The compounds of the present disclosure may possess a sufficiently acidic, a sufficiently basic, or both functional groups, can react with any of a number of inorganic bases, and inorganic and organic acids, to form a salt. Alternatively, compounds that are inherently charged, such as those with a quaternary nitrogen, can form a salt with an appropriate counterion, e.g, a halide such as bromide, chloride, or fluoride.

In certain embodiments, compounds or salts of Formula (I), (I-a), or (II), may be prodrugs. The term “prodrug” is intended to encompass compounds which, under physiologic conditions, are converted into pharmaceutical agents of the present disclosure. One method for making a prodrug is to include one or more selected moieties which are hydrolyzed under physiologic conditions to reveal the desired molecule. In other embodiments, the prodrug is converted by an enzymatic activity of the host animal such as specific target cells in the host animal.

In some embodiments, the design of a prodrug increases the lipophilicity of the pharmaceutical agent. In some embodiments, the design of a prodrug increases the effective water solubility. See, e.g, Fedorak et al, Am. J. Physiol, 269:G210-218 (1995), McLoed et al, Gastroenterol, 106:405-413 (1994): Hochhaus et al, Biomed. Chrom. 6:283-286 (1992): J. Larsen and H. Bundgaard, Int. J. Pharmaceutics. 37, 87 (1987); J. Larsen et al, Int. J Pharmaceutics, 47, 103 (1988); Sinkula et al, J. Pharm. Sci, 64:181-210 (1975): T. Higuchi and V. Stella, Pro-drugs as Novel Delivery Systems, Vol. 14 of the A.C.S. Symposium Series; and Edward B. Roche, Bioreversible Carriers in Drug Design, American Pharmaceutical Association and Pergamon Press, 1987, all incorporated herein for such disclosure). According to another embodiment, the present disclosure provides methods of producing the above-defined compounds. The compounds may be synthesized using conventional techniques. Advantageously, these compounds are conveniently synthesized from readily available starting materials.

Synthetic chemistry transformations and methodologies useful in synthesizing the compounds described herein are known in the art and include, for example, those described in R. Larock, Comprehensive Organic Transformations (1989): T. W. Greene and P. G. M. Wuts, Protective Groups in Organic Synthesis. 2d. Ed. (1991): L. Fieser and M. Fieser, Fieser and Fieser's Reagents for Organic Synthesis (1994); and L. Paquette. ed, Encyclopedia of Reagents for Organic Synthesis (1995).

### Pharmaceutical Formulations

In some aspects, the present disclosure provides a pharmaceutical composition comprising a compound or salt of Formula (I), (I-a), or (II) and at least one pharmaceutically acceptable excipient. In some embodiments, the pharmaceutical composition comprises a compound or salt of Formula (I), (I-a), or (II) and a pharmaceutically acceptable excipient.

Pharmaceutical compositions can be formulated using one or more physiologically-acceptable carriers comprising excipients and auxiliaries. Formulation can be modified depending upon the route of administration chosen. Pharmaceutical compositions comprising a compound, salt or conjugate can be manufactured, for example, by lyophilizing the compound, salt or conjugate, mixing, dissolving, emulsifying, encapsulating or entrapping the conjugate. The pharmaceutical compositions can also include the compounds, salts or conjugates in a free-base form or pharmaceutically-acceptable salt form.

Pharmaceutical compositions as often further can comprise more than one active compound (e.g, a compound, salt or conjugate and other agents) as necessary for the particular indication being treated. The active compounds can have complementary activities that do not adversely affect each other. Such molecules can be present in combination in amounts that are effective for the purpose intended.

A compound or salt of any one of Formula (I), (1-a), or (II) may be formulated in any suitable pharmaceutical formulation. A pharmaceutical formulation of the present disclosure typically contains an active ingredient (e.g, compound or salt of any one Formula I) and one or more pharmaceutically acceptable excipients or carriers, including but not limited to: inert solid diluents and fillers, diluents, sterile aqueous solution and various organic solvents, permeation enhancers, antioxidents, solubilizers, and adjuvants.

In certain embodiments, a compound or salt of Formula (I), (I-a), or (II) is formulated with a chelating agent or other material capable of binding metal ions, such as ethylene diamine tetra acetic acid (EDTA) and its salts are capable of enhancing the stability of a compound or salt of Formula (I), (I-a), or (II).

Pharmaceutical formulations may be provided in any suitable form, which may depend on the route of administration.

In some embodiments, the disclosure provides a pharmaceutical composition for oral administration containing at least one compound or salt of any one of Formula (I), (I-a), or (II) and a pharmaceutical excipient suitable for oral administration. The composition may be in the form of a solid, liquid, gel, semi-liquid, or semi-solid. In some embodiments, the composition further comprises a second agent.

Pharmaceutical compositions of the disclosure suitable for oral administration can be presented as discrete dosage forms, such as hard or soft capsules, cachets, troches, lozenges, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion, or dispersible powders or granules, or syrups or elixirs. Such dosage forms can be prepared by any of the methods of pharmacy, which typically include the step of bringing the active ingredient(s) into association with the carrier. In general, the composition are prepared by uniformly and intimately admixing the active ingredient(s) with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient(s) in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound or salt of any one of Formula (I), (I-a), or (II) moistened with an inert liquid diluent.

Pharmaceutical compositions may also be prepared from a compound or salt of any one of Formula (I), (I-a), or (II) and one or more pharmaceutically acceptable excipients. Preparations for such pharmaceutical composition are well-known in the art. See, e.g, Anderson, Philip O.; Knoben, James E.; Troutman, William G, eds, Handbook of Clinical Drug Data, Tenth Edition, McGraw-Hill, 2002; Pratt and Taylor, eds, Principles ofDrugAction, Third Edition, Churchill Livingston, New York, 1990; Katzung, ed, Basic and Clinical Pharmacology, Ninth Edition, McGraw Hill, 2003; Goodman and Gilman, eds, The Pharmacological Basis of Therapeutics, Tenth Edition, McGraw Hill, 2001: Remingtons Pharmaceutical Sciences, 20th Ed, Lippincott Williams & Wilkins, 2000; Martindale, The Extra Pharmacopoeia, Thirty-Second Edition (The Pharmaceutical Press, London, 1999).

### Methods of Treatment

In some aspects, the present disclosure provides a method of modulating IL-17 A in a subject in need thereof, comprising administering to the subject a compound or salt of Formula (I), (I-a), or (II) or a pharmaceutical composition thereof.

In some aspects, the present disclosure provides a method of a method of treating an inflammatory disease or condition in a subject in need thereof, comprising administering to the subject a compound or salt of Formula (I), (I-a), or (II) or a pharmaceutical composition thereof. In some embodiments, the inflammatory disease or condition is selected from plaque psoriasis, guttate psoriasis, inverse psoriasis, pustular psoriasis, erythrodermic psoriasis, psoriatic arthritis, ankylosing spondylitis, hidradenitis suppurativa, rheumatoid arthritis, Palmoplantar Psoriasis, Spondyloarthritis, and Non-infectious Uveitis.

In certain embodiments, a compound or salt of Formula (I), (I-a), or (II), can be used to treat or prevent a disease or condition that is mediated directly or indirectly by IL-17A. Such diseases include inflammatory diseases and conditions, proliferative diseases (e.g, cancer), autoimmune diseases and other disease described herein. The methods generally involve administering therapeutically effective amounts of compounds disclosed herein or a pharmaceutical composition thereof to the subject.

Increased levels of IL-17A have been associated with several conditions including airway inflammation, rheumatoid arthritis (R^{A}), osteoarthritis, bone erosion, intraperitoneal abscesses and adhesions, inflammatory bowel disorder (IBD), allograft rejection, psoriasis. psoriatic arthritis. ankylosing spondylitis, certain types of cancer, angiogenesis, atherosclerosis and multiple sclerosis (MS). Both IL-17A and IL-17R are upregulated in the synovial tissue of R^{A} patients. IL-17A exerts its role in pathogenesis of R^{A} through IL-1-β and TNF-α dependent and independent pathways. IL-17A stimulates secretion of other cytokines and chemokines, e.g, TNF-α, IL-10β, IL-6, IL-8 and Gro-α. IL-17A directly contributes to disease progression in R^{A}. Injection of IL-17A into the mouse knee promotes joint destruction independently of IL-1 β activity (Ann Rheum Dis 2000, 59:529-32). Anti-IL-1β antibody has no effect on IL-17A induced inflammation and joint damage (J. Immunol 2001, 167:1004-1013). In a streptococcal cell wall (SCW)-induced murine arthritis model. IL-17A induced inflammatory cell infiltration and proteoglycan depletion in wild-type and IL-1β knockout and TNF-α knockout mice. IL-17A knockout mice are phenotypically normal in the absence of antigenic challenge but have markedly reduced arthritis following type II collagen immunization (J. Immunol 2003, 171:6173-6177). Increased levels of IL-17A-secreting cells have also been observed in the facet joints of patients suffering from ankylosing spondylitis (H Appel et al, Arthrntis Res Therap. 2011. 13:R⁹⁵).

Multiple sclerosis is an autoimmune disease characterized by central nervous system (CNS) inflammation with damage to the myelin sheath surrounding axons. A hallmark of MS is that T cells infiltrate into the CNS. Higher numbers of IL-17A mRNA-expressing blood mono-nuclear cells (MNC) are detected during MS clinical exacerbation compared to remission (Multiple Sclerosis. 5:101-104, 1999). Furthermore, experimental autoimmune encephalomyelitis (“EAE”), a preclinical animal model for MS is significantly suppressed in IL-17A knockout mice.

In certain aspects, the disclosure provides methods of modulating IL-17A in a subject in need thereof, comprising administering to said subject a compound or salt of Formula (I), (I-a), or (II). In certain embodiments, a compound or salt of Formula (I), (1-a), or (II) inhibits the activity of IL-17A in a subject in need thereof.

In certain embodiments, a compound or salt of Formula (I), (I-a), or (II) is used to treat or prevent an inflammatory disease or condition. In certain embodiments, a compound or salt of Formula (I), (I-a), or (II) is administered to a subject in need thereof to treat an inflammatory disease or condition, e.g, psoriasis.

In certain embodiments, a compound or salt of Formula (I), (I-a), or (II) is used to treat or prevent an inflammatory disease or condition is selected from, plaque psoriasis, guttate psoriasis, inverse psoriasis, pustular psoriasis, erythrodermic psoriasis. psoriatic arthritis. ankylosing spondylitis, hidradenitis suppurativa, rheumatoid arthritis, Palmoplantar Psoriasis, Spondyloarthritis, and Non-infectious Uveitis. In certain embodiments, a compound or salt of Formula (I), (I-a), or (II) is used to treat or prevent psoriasis. In certain embodiments, a compound or salt of Formula (I), (I-a), or (II) is used for the treatment or prevention of a condition including, but not limited to, airway inflammation, ankylosing spondylitis, asthma, R^{A} (including juvenile R^{A}), as well as other inflammatory disorders, conditions, or diseases.

### EXAMPLES

The invention now being generally described, it will be more readily understood by reference to the following examples which are included merely for purposes of illustration of certain aspects and embodiments of the present invention, and are not intended to limit the invention in any way.

The following synthetic schemes are provided for purposes of illustration, not limitation. The following examples illustrate the various methods of making compounds described herein. It is understood that one skilled in the art may be able to make these compounds by similar methods or by combining other methods known to one skilled in the art. It is also understood that one skilled in the art would be able to make, in a similar manner as described below by using the appropriate starting materials and modifying the synthetic route as needed. In general, starting materials and reagents can be obtained from commercial vendors or synthesized according to sources known to those skilled in the art or prepared as described herein.

General Procedures and Examples 1-176 show general and exemplary procedures for the preparation of the claimed IL-17A modulators. Example 177 provides IL-17 A/A bioassay inhibition data.

### General Procedures

General Procedure A: To a solution of Boc-amine (1 eq) in DCM (0.1-0.5 M) was added trifluoroacetic acid (equal volume to DCM or ˜20 eq). The reaction mixture was stirred until the disappearance of Boc-amine starting material. The reaction mixture is then concentrated and purified by prep-HPLC or silica gel column chromatography to afford the corresponding amine.

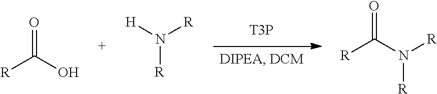

General Procedure B: To a solution of carboxylic acid (1.0 eq) in DCM (0.1-0.5 M) cooled to 0° C. is added DIPEA (15 eq) followed by a solution of T3P in EtOAc or ACN (2-10 eq), dropwise. The reaction is allowed to warm to RT and stir until the disappearance of the carboxylic acid starting material, 30 min-16 h. The reaction mixture was diluted with H₂O and extracted with EtOAc. The combined organic layers were washed with brine, dried over Na2SO4, filtered, and concentrated under reduced pressure to afford a crude residue, which was purified by silica gel chromatography or prep-HPLC to afford the corresponding amide.

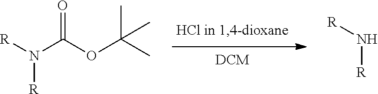

General Procedure C: To a solution of tert-butyl carbamate (1 eq) in DCM (0.2 M) cooled in an ice bath was added 4 N HCl in 1,4-dioxane, dropwise. The reaction was allowed to warm to RT and stir until the starting material has disappeared. The reaction mixture was concentrated under reduced pressure and purified by prep-HPLC (ACN in buffered water) to afford the amine.

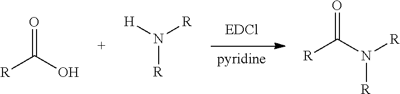

General Procedure D: To a solution of carboxylic acid (1 eq) and amine (1.5 eq) in pyridine (0 l M) was added EDCI (3 eq). The reaction mixture was stirred at RT for 1-18 h. The reaction mixture was diluted with H₂O, extracted with EtOAc, dried over anhydrous Na2SO₄, filtrated, and concentrated under vacuum. The residue was punfied by prep-HPLC or silica gel chromatography to afford the desired amide.

### Example 1: General Scheme C-Synthesis of Compounds 1-67

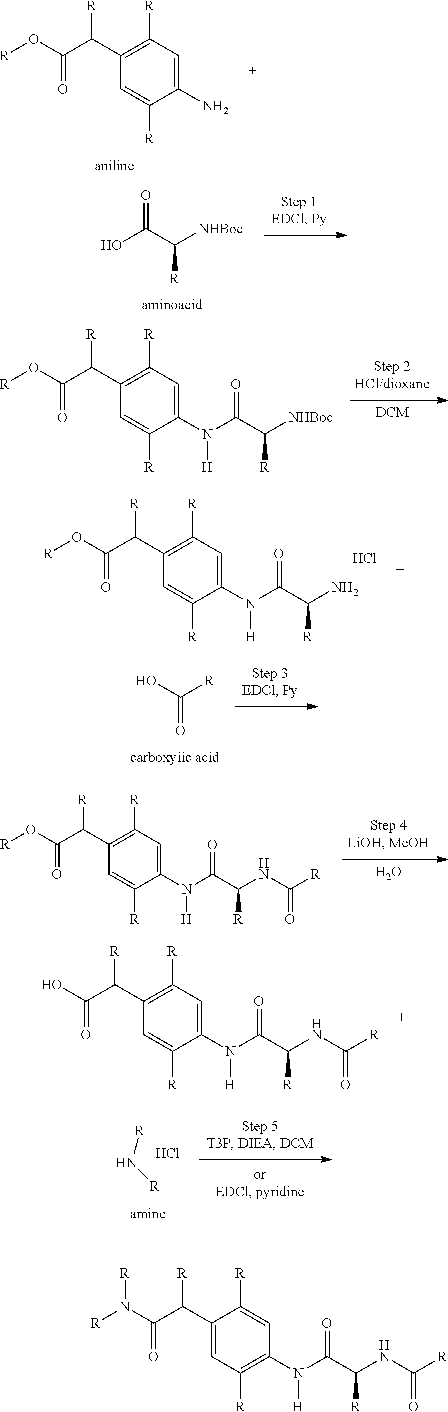

Compounds synthesized according to General Scheme C employ an aniline and amino acid in step one followed by a carboxylic acid in step 3 and an amine in step 5. Starting materials are listed in order of use.

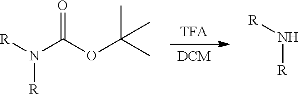

### Example 2: Synthesis of (S)-N-(1-cyclohentyl-2-((2-fluoro-4-(2-(methyl(22.2-trifluoroethyl)amino)-2-oxoethyl)phenyl)amino)-2-oxoethyl)-1-ethyl-1H-pyrazole-5-carboxamide (1)

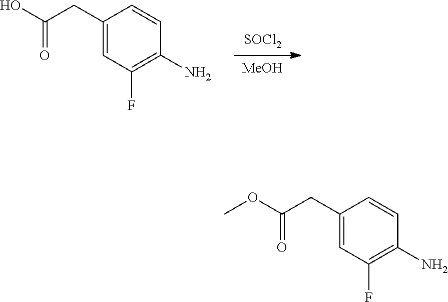

Step 1—Synthesis of Methyl 2-(4-amino-3-fluorophenyl)acetate: To a solution of 2-(4-amino-3-fluorophenyl)acetic acid (300 mg, 1.77 mmol, 1.00 eq) in MeOH (4.00 mL) was added drop-wise SOC12 (253 mg, 2.13 mmol, 154 μL, 1.20 eq). The mixture was stirred at 60° C. for 3 h.

The reaction mixture was concentrated under reduced pressure to afford methyl 2-(4-amino-3-fluorophenyl)acetate (300 mg, 1.6 mmol, 92% yield) as a yellow solid. LCMS [M+H]⁺=184.2 m±

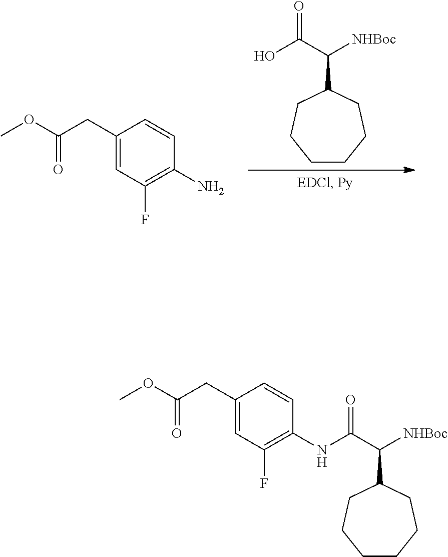

Step 2—Synthesis of Methyl (S)-2-(4-(2-((tert-butoxycarbonyl)amino)-2-cycloheptylacetamido)-3-fluorophenyl)acetate: To a solution of methyl 2-(4-amino-3-fluorophenyl)acetate (300 mg, 1.64 mmol, 1.00 eq) and (S)-2-((tert-butoxycarbonyl)amino)-2-cycloheptylacetic acid (533 mg, 1.97 mmol, 1.20 eq) in Py (5.00 mL) was added EDCI (941 mg, 4.91 mmol, 3.00 eq). The mixture was stirred at RT for 12 h. The reaction mixture was concentrated under reduced pressure, diluted with H₂O, and extracted with EtOAc. The combined organic layers were dried over Na2SO4, filtered, and concentrated under reduced pressure to afford methyl (S)-2-(4-(2-((tert-butoxycarbonyl)amino)-2-cycloheptylacetamido)-3-fluorophenyl)acetate (600 mg, 1.4 mmol, 84% yield) as a yellow solid. LCMS [M+H]⁺=437.3 m/z. ¹H NMR (400 MHz, DMS₀-dr,): 69.65-9.08(m, 1H), 7.81-7.68 (m, 1H), 7.17 (dd, J, =1.6 Hz, J₂=11.6 Hz, 1H), 7.05 (d, J=8.0 Hz, 1H), 6.87 (d, =8.8 Hz, 11H), 4.11 (t,/ =8.0 Hz, 1H), 3.68-3.62 (m, 5H), 1.93-1.87 (m, 1H), 1.70-1.50 (m, 8H), 1.41-1.36 (m, 13H).

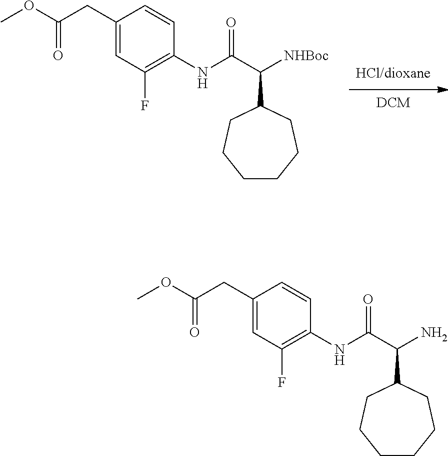

Step 3—Synthesis of Methyl (S)-2-(4-(2-amino-2-cycloheptylacetamido)-3-fluorophenyl)acetate: To a solution of methyl (S)-2-(4-(2-((tert-butoxycarbonyl)amino)-2-cycloheptylacetamido)-3-fluorophenyl)acetate (600 mg, 1.37 mmol, 1.00 eq) in DCM (5.00 mL) was added HCl/dioxane (4.00 M, 3.44 mL, 10.0 eq) at 0° C. The mixture was stirred at RT for 2 h. The reaction mixture was concentrated to afford methyl (S)-2-(4-(2-amino-2-cycloheptylacetamido)-3-fluorophenyl)acetate (450 mg, 1.2 mmol, 88% yield. HCl) as a yellow solid. LCMS JM+Hj =337.3 m/z.

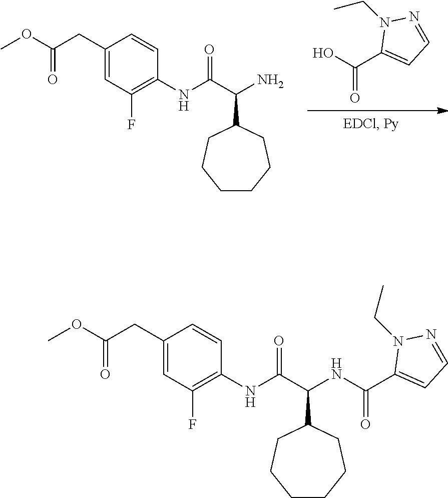

Step 4—Synthesis of Methyl (S)-2-(4-(2-cycloheptyl-2-(1-ethyl-1H-pyrazole-5-carboxamido)acetamido)-3-fluorophenyl)acetate: To a solution of methyl (S)-2-(4-(2-amino-2-cycloheptylacetamido)-3-fluorophenyl)acetate (450 mg, 1.21 mmol, 1.00 eq, HCl) and 1-ethyl-1H-pyrazole-5-carboxylic acid (253 mg, 1.81 mmol, 1.50 eq) in Py (10 mL) was added EDCI (694 mg, 3.62 mmol, 3.00 eq). The mixture was stirred at RT for 12 h. The reaction mixture was concentrated under reduced pressure, diluted with H₂O, and extracted with EtOAc. The combined organic layers were washed with sat. aqueous NH₄Cl, dried over Na2SO4, filtered, and concentrated under reduced pressure to give a residue. The residue was purified by silica gel chromatography (SiO₂, petroleum ether: EtoAc=100: 1 to 1: 1) to afford methyl (S)-2-(4-(2-cycloheptyl-2-(1-ethyl-1H-pyrazole-5-carboxamido)acetamido)-3-fluorophenyl)acetate (420 mg, 920 umol, 76% yield) as a white solid. LCMS [M+H]⁺=459.4 m/z. H NMR (400 MHz, DMS₀-d₆): δ 9.91 (s, 1H), 8.45 (d, J=8.4 Hz, 1H), 7.70 (t, J=8.4 Hz, 1H), 7.47 (d, J=2.0 Hz, 1H), 7.18 (dd, J₁=1.6 Hz, J₂=11.6 Hz, 1H), 7.06 (dd, J1=1.2 Hz, J2=8.4 Hz, 1H), 6.99 (d. J=2.0 Hz, 1H), 4.62 (t, J=8.4 Hz, 11H), 4.47 (q, J=7.2 Hz, 2H), 3.69 (s, 2H), 3.62 (s, 3H), 2.12-2.09 (m, 1H), 1.74-1.38 (m, 12H), 1.28 (t. =7.2 Hz, 3H).

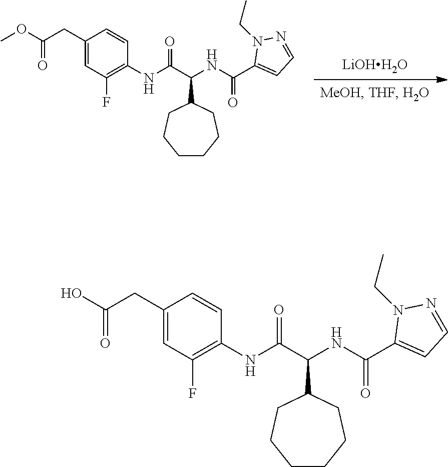

Step 5—Synthesis of (S)-2-(4-(2-cycloheptyl-2-(1-ethyl-1H-pyrazole-5-carboxamido)acetamido)-3-fluorophenyl)acetic acid: To a solution of methyl (S)-2-(4-(2-cycloheptyl-2-(1-ethyl-1H-pyrazole-5-carboxamido)acetamido)-3-fluorophenyl)acetate (100 mg, 218 umol, 1.00 eq) in MeOH (4.00 mL) was added a solution of LiOH—H₂O (10.9 mg, 261 umol, 1.20 eq) in H₂O (1.00 mL) at 0° C. The mixture was stirred at RT for 2 h. The reaction mixture was diluted with H₂O, extracted with EtOAc, dried over Na₂SO₄, filtered, and concentrated under reduced pressure to afford (S)-2-(4-(2-cycloheptyl-2-(1-ethyl-1H-pyrazole-5-carboxamido)acetamido)-3-fluorophenyl)acetic acid (95 mg, 210 umol, 98% yield) as a white solid.

LCMS [M+H]⁺=445.3 m/z. ¹H NMR (400 MHz, DMS₀-d₆): δ 12.39 (s, 1H), 9.89 (s, 1H), 8.45 (d, J=8.8 Hz, 1H), 7.67 (t, J=8.4 Hz, 1H), 7.47 (d, J=2.0 Hz, 1H), 7.16 (dd, J₁=1.6 Hz, J₂=11.6 Hz, 1H), 7.04 (dd, J₁=1.2 Hz, J₂=8.4 Hz, 1H), 6.99 (d. J=2.4 Hz, 1H), 4.61 (t, =8.4 Hz, 1H), 4.47 (q, J=7.2 Hz, 2H), 3.57 (s, 2H), 2.12-2.09 (m, 1H), 1.73-1.35 (m, 12H), 1.28 (t, J=7.2 Hz, 3H).

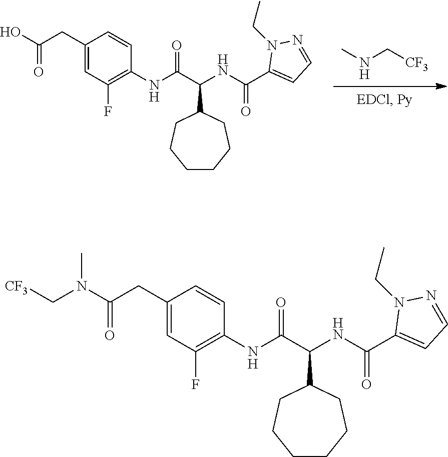

Step 6—Synthesis of (S)-N-(1-cycloheptyl-2-((2-fluoro-4-(2-(methyl(2,2,2-trifluoroethyl)amino)-2-oxoethyl)phenyl)amino)-2-oxoethyl)-1-ethyl-1H-pyrazole-5-carboxamide (1). To a solution of (S)-2-(4-(2-cycloheptyl-2-(1-ethyl-1 H-pyrazole-5-carboxamido)acetamido)-3-fluorophenyl)acetic acid (95.0 mg, 213 umol, 1.00 eq) and 2,2,2-trifluoro-N-methylethan-1-amine hydrochloride (63.9 mg, 427 umol, 2.00 eq. HCl) in Py (5.00 mL) was added EDCI (122 mg, 641 umol, 3.00 eq). The mixture was stirred at RT for 12 h. The reaction mixture was diluted with H₂O and extracted with DCM. The combined organic layers were washed with sat. aqueous NaHCO₃, dried over Na₂SO₄. filtered, and concentrated under reduced pressure to give a residue. The residue was purified by Prep-TLC (SiO₂. DCM: MeOH=10: 1, TLC, Plate 1, DCM: MeOH=10: 1, Rf=0.5) and prep-HPLC (basic condition, column: Phenomenex Gemini-NX C18 75×30 mm×3 um; mobile phase: [water (0.05% ammonia hydroxide v/v)—ACN]; B %: 28%-58%, 7 min). (S)-N-(1-cycloheptyl-2-((2-fluoro-4-(2-(methyl(2,2,2-trifluoroethyl)amino)-2-oxoethyl)phenyl)amino)-2-oxoethyl)-1-ethyl-1H-pyrazole-5-carboxamide (44 mg, 80 umol, 37% yield) was obtained as a white solid. LCMS [M+H]⁺=540.4 m/z.

### Example 3: Synthesis of Compound 2

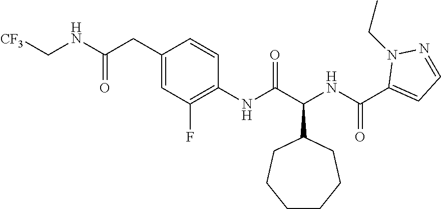 [SMILES: CCn1nccc1C(=O)N[C@H](C(=O)Nc1ccc(CC(=O)NCC(F)(F)F)cc1F)C1CCCCCC1]

(S)-N-(1-cycloheptyl-2-((2-fluoro-4-(2-oxo-2-((2,2,2-trifluoroethyl)amino)ethyl)phenyl) amino)-2-oxoethyl)-1-ethyl-1H-pyrazole-5-carboxamide (2). Prepared according to General Scheme C, employing ethyl 2-(4-amino-3-fluorophenyl)acetate and (S)-2-((tert-butoyvcarbonyl)amino)-2-cycloheptylacetic acid followed by 1-ethyl-1H-pyrazole-5-carboxylic acid and 2,2,2-trifluoroethan-1-amine hydrochloride. LCMS [M+H]⁺=526.4 m.

### Example 4: Synthesis of Compound 3

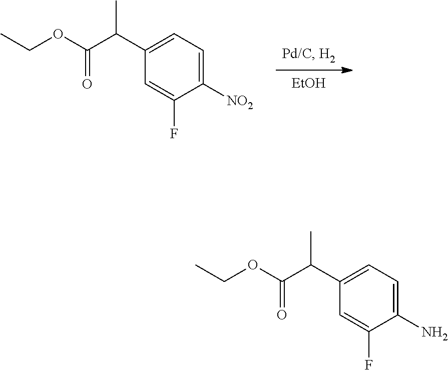 [SMILES: "EtOH" ; CCOC(=O)C(C)c1ccc([N+](=O)[O-])c(F)c1 ; CCOC(=O)C(C)c1ccc(N)c(F)c1]

Ethyl 2-(4-amino-3-fluorophenyl)propanoate. To a solution of ethyl 2-(3-fluoro-4-nitrophenyl)propanoate (1.00 g, 4.15 mmol, 1.00 eq) in EtOH (10.0 mL) was added Pd/C (100 mg, 10% purity) under N₂. The suspension was degassed under vacuum and purged with H₂ several times. The mixture was stirred under an atmosphere of hydrogen gas at 15 PSI at 15° C. for 15 h. The reaction mixture was filtered, and the filtrate was concentrated to afford ethyl 2-(4-amino-3-fluorophenyl)propanoate (0.85 g, 4.0 mmol, 97% yield) as brown oil. LCMS [M+H]⁺=212.0 m/z.

¹H NMR (400 MHz, CDCl₃): δ 6.98 (dd, J₁=2.0 Hz, J₂=12.0 Hz, 1H), 6.89 (dd, J¹=2.0 Hz, J.2=8.4 Hz, 1H), 6.80-6.73 (m, 1H), 4.21-4.05 (m, 2H), 3.64-3.52 (m, 1H), 1.45 (d, J=7.2 Hz, 3H), 1.22 (t, J=7.2 Hz, 3H).

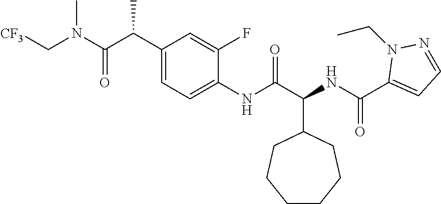 [SMILES: CCn1nccc1C(=O)N[C@H](C(=O)Nc1ccc([C@@H](C)C(=O)N(C)CC(F)(F)F)cc1F)C1CCCCCC1]

N-[(1S)-1-cycloheptyl-2-[2-fluoro-4-[(1R)-1-methyl-2-[methyl(2,2,2-trifluoroethyl)amino]-2-oxo-ethyl]anilino]-2-oxo-ethyl]-2-ethyl-pyrazole-3-carboxamide (3). Prepared according to General Scheme C, employing ethyl 2-(4-amino-3-fluorophenyl)propanoate and (S)-2-((tert-butoxycarbonyl)amino)-2-cycloheptylacetic acid followed by 1-ethyl-1H-pyrazole-5-carboxylic acid and 2,2,2-trifluoro-N-methylethan-1-amine hydrochloride. The title compound was isolated as the first eluting, single stereoisomer by chiral SFC purification (column: DAICEL CHIRALPAK AD—H (250 mm×30 mm, 5 um); mobile phase: 30% [0.1% NH₃H₂O in IPA]). LCMS [M+H]⁺=554.3 m/z. Absolute stereochemistry at the benzylic center was assigned by comparison to the small molecule x-ray of Example 4.

### Example 5: Synthesis of Compound 4

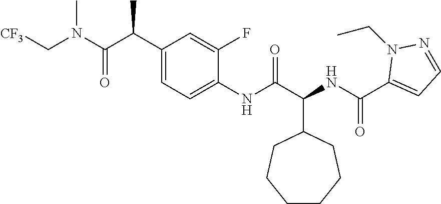 [SMILES: CCn1nccc1C(=O)N[C@H](C(=O)Nc1ccc([C@H](C)C(=O)N(C)CC(F)(F)F)cc1F)C1CCCCCC1]

N-((S)-1-cycloheptyl-2-((2-fluoro-4-((S)-1-(methyl(2,2,2-trifluoroethyl)amino)-1-oxopropan-2-yl)phenyl)amino)-2-oxoethyl)-1-ethyl-1H-pyrazole-5-carboxamide (4). Prepared according to General Scheme C, employing ethyl 2-(4-amino-3-fluorophenyl)propanoate and (S)-2-((tert-butoxycarbonyl)amino)-2-cycloheptylacetic acid followed by 1-ethyl-1H-pyrazole-5-carboxylic acid and 2,2,2-trifluoro-N-methylethan-1-amine hydrochloride. The title compound was isolated as the second eluting, single stereoisomer by chiral SFC purification (column: DAICEL CHIRALPAK AD—H (250 mm×30 mm, 5 um); mobile phase: 30% [0.1% NH-H₂O in IPA]).

LCMS [M+H]⁺=554.3 m/z. Absolute stereochemistry was determined by small molecule x-ray of a single crystal obtained by slow evaporation with 1:3 EtOAc/ACN.

### Example 6: Synthesis of Compound 5

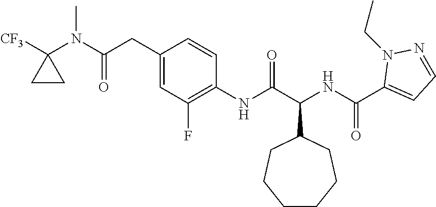 [SMILES: CCn1nccc1C(=O)N[C@H](C(=O)Nc1ccc(CC(=O)N(C)C2(C(F)(F)F)CC2)cc1F)C1CCCCCC1]

(S)-N-(1-cycloheptyl-2-((2-fluoro-4-(2-(methyl(1-(trifluoromethyl)cyclopropyl)amino)-2-oxoethyl)phenyl)amino)-2-oxoethyl)-1-ethyl-1H-pyrazole-5-carboxamide (5). Prepared according to General Scheme C. employing ethyl 2-(4-amino-3-fluorophenyl)acetate and (S)-2-((tert-butoxycarbonyl)amino)-2-cycloheptylacetic acid followed by 1-ethyl-1H-pyrazole-5-carboxylic acid and N-methyl-1-(trifluoromethyl)cyclopropan-1-amine hydrochloride. LCMS [M+H]⁺=566.3 m/z.

### Example 7: Synthesis of Compound 6

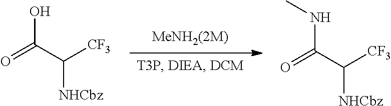 [SMILES: O=C(NC(C(=O)O)C(F)(F)F)OCc1ccccc1 ; "T3P, DIEA, DCM" ; CNC(=O)C(NC(=O)OCc1ccccc1)C(F)(F)F]

Benzyl (1,1,1-trifluoro-3-(methylamino)-3-oxopropan-2-yl)carbamate. To a solution of 2-(((benzyloxy)carbonyl)amino)-3,3,3-trifluoropropanoic acid (100 mg, 360 umol, 1.00 eq), MeNH₂ (2.00 M, 3.61 mL, 20.0 eq) in DCM (2.00 mL) was added DIEA (233 mg, 1.80 mmol, 314 μL, 5.00 eq) and T₃P in EtOAc (459 mg, 721 umol, 429 μL, 50% purity, 2.00 eq). The mixture was stirred at RT for 2 h. The reaction mixture was diluted with H₂O and extracted with EtOAc. The combined organic layers were washed with brine, dried over Na₂SO₄, filtered, and concentrated under reduced pressure to afford crude benzyl (1,1,1-trifluoro-3-(methylamino)-3-oxopropan-2-yl)carbamate (40.0 mg) as a yellow solid. LCMS [M+H]⁺=313.2 m/z.

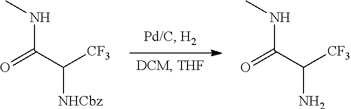

2-amino-3,3,3-trifluoro-N-methylpropanamide. To a solution of 1,1,1-trifluoro-3-(methylamino)-3-oxopropan-2-yl)carbamate (40.0 mg, 137 umol, 1.00 eq) in DCM (2.00 mL) and THF (2.00 mL) was added Pd/C (0.050 g, 137 umol, 10% purity, 1.00 eq) under N₂ atmosphere.

The mixture was stirred under H₂ (15 Psi) at 15° C. for 2 hrs. TLC (plate 1, petroleum ether: ethyl acetate=1: 1, Rr(R 1)=0.40) showed 1,1,1-trifluoro-3-(methylamino)-3-oxopropan-2-yl)carbamate was consumed completely and new spots were detected. The mixture was filtered to afford crude 2-amino-3,3,3-trifluoro-N-methylpropanamide (20 mg) as a light yellow solid, which was used directly in the next reaction.

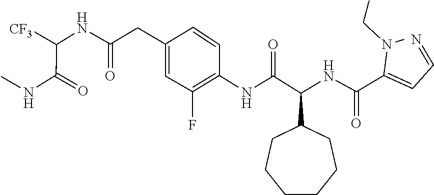

N-((1S)-1-cycloheptyl-2-((2-fluoro-4-(2-oxo-2-((1,1,1-trifluoro-3-(methylamino)-3-oxopropan-2-yl)amino)ethyl)phenyl)amino)-2-oxoethyl)-1-ethyl-1H-pyrazole-5-carboxamide (6). Prepared according to General Scheme C, employing ethyl 2-(4-amino-3-fluorophenyl)acetate and (S)-2-((tert-butoxycarbonyl)amino)-2-cycloheptylacetic acid followed by 1-ethyl-1H-pyrazole-5-carboxylic acid and 2-amino-3,3,3-trifluoro-N-methylpropanamide. LCMS [M+H]⁺=583.5 m.

### Example 8: Synthesis of Compound 7

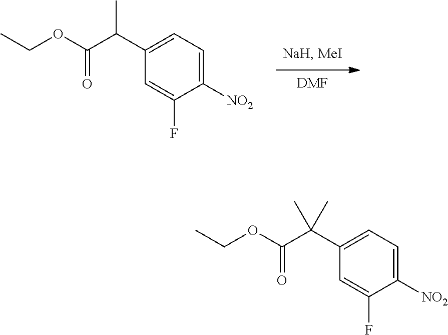

Ethyl 2-(3-fluoro-4-nitrophenyl)-2-methylpropanoate. To a solution of ethyl 2-(3-fluoro-4-nitrophenyl)propanoate (1.00 g, 4.15 mmol, 1.00 eq) in DMF (3.00 mL) was added NaH (249 mg, 6.22 mmol, 60% purity, 1.50 eq) at 0° C., then methyl iodide (706 mg, 4.97 mmol, 309 μL, 1.20 eq) was added. The reaction mixture was stirred at 0° C. for 10 min. Hydrochloric acid (0 M, 20.0 mL) was added to the reaction mixture at 0° C. The reaction mixture was then diluted with H₂O and extracted with EtOAc. The combined organic layers were dried over Na₂SO₄, filtered, and concentrated under reduced pressure to afford ethyl 2-(3-fluoro-4-nitrophenyl)-2-methylpropanoate (1.0 g, 4.1 mmol, 99% yield) as a yellow oil. ¹H NMR (400 MHz, CDCl₃): δ 8.06-8.02 (m, 1H), 7.30-7.25(m, 2H), 4.18-4.13 (m, 2H), 1.60 (s, 6H), 1.23 (t, J=3.2 Hz, 3H).

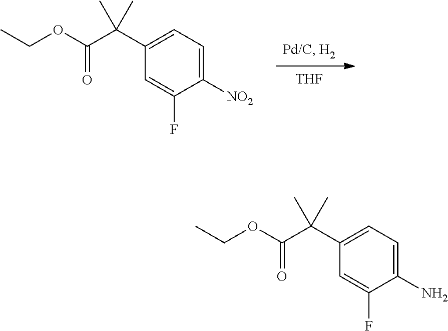 [SMILES: CCOC(=O)C(C)(C)c1ccc([N+](=O)[O-])c(F)c1 ; CCOC(=O)C(C)(C)c1ccc(N)c(F)c1 ; "THF"]

Ethyl 2-(4-amino-3-fluorophenyl)-2-methylpropanoate. A mixture of ethyl 2-(3-fluoro-4-nitrophenyl)-2-methylpropanoate (1.05 g, 4.11 mmol, 1.00 eq), Pd/C (0.100 g, 10% purity) in EtOH (15.0 mL) was stirred at rt for 3 h under a hydrogen gas atmosphere at 15 Psi. The reaction mixture was filtered and concentrated under reduced pressure to afford ethyl 2-(4-amino-3-fluorophenyl)-2-methylpropanoate (0.92 g, 4.1 mmol, 99% yield) as a yellow oil. LCMS [M+H]⁺=226.0 m/z.

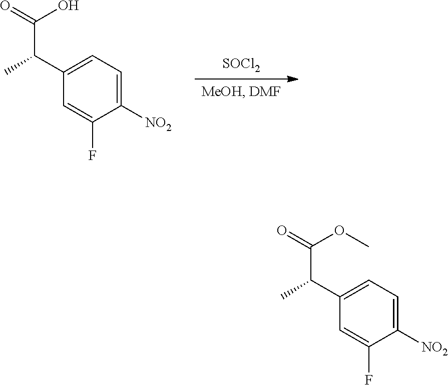

Synthesis of methyl (S)-2-(3-fluoro-4-nitrophenyl)propanoate To a solution of (S)-2-(3-fluoro-4-nitrophenyl)propanoic acid (0.500 g, 2.35 mmol, 1.00 eq) in MeOH (5.00 mL), DMF (0.500 mL) was added SOCl₂ (837 mg, 7.04 mmol, 510 μL, 3.00 eq). The mixture was stirred at 0° C. for 1 h. The reaction mixture was concentrated under reduced pressure to give a residue, and then the residue was diluted with H₂O and extracted with ethyl acetate, dried over Na₂SO₄, filtered, and concentrated under reduced pressure to afford methyl (S)-2-(3-fluoro-4-nitrophenyl)propanoate (0.50 g, 2.2 mmol, 4% yield) as a yellow oil.

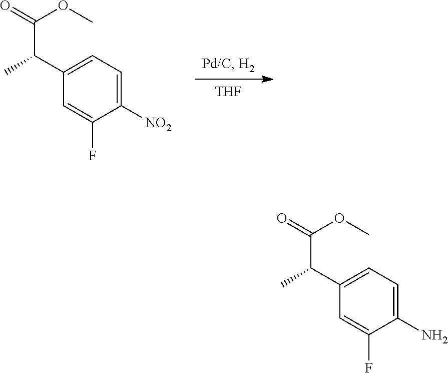 [SMILES: COC(=O)[C@@H](C)c1ccc([N+](=O)[O-])c(F)c1 ; "Pd/C, H2" ; COC(=O)[C@@H](C)c1ccc(N)c(F)c1 ; "THF"]

Synthesis of methyl (S)-2-(4-amino-3-fluorophenyl)propanoate To a solution of methyl (S)-2-(3-fluoro-4-nitrophenyl)propanoate (0.500 g, 2.20 mmol, 1.00 eq) in THF (5.00 mL) was added Pd/C (10%, 0.10 g) under N₂ atmosphere. The mixture was stirred under H₂ at 15 PSI at RT for 2 h. The reaction mixture was concentrated under reduced pressure to afford crude methyl (S)-2-(4-amino-3-fluorophenyl)propanoate (0.40 g) as a yellow oil. LCMS [M+H]⁺=198.0 m/z.

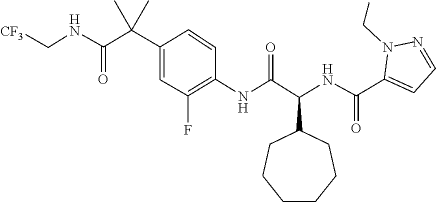

(S)-N-(1-cycloheptyl-2-((2-fluoro-4-(2-methyl-1-oxo-1-((2,2,2-trifluoroethyl)amino)propan-2-yl)phenyl)amino)-2-oxoethyl)-1-ethyl-1H-pyrazole-5-carboxamide (7). Prepared according to General Scheme C, employing ethyl 2-(4-amino-3-fluorophenyl)-2-methylpropanoate and (S)-2-((tert-butoxycarbonyl)amino)-2-cycloheptylacetic acid followed by 1-ethyl-1H-pyrazole-5-carboxylic acid and 2,2,2-trifluoroethan-1-amine hydrochloride. LCMS [M+H]⁺=554.4 m,-.

### Example 9: Synthesis of Compound 8

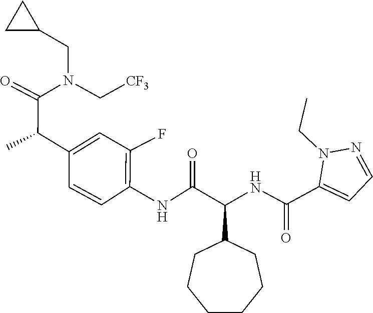 [SMILES: CCn1nccc1C(=O)N[C@H](C(=O)Nc1ccc([C@H](C)C(=O)N(CC2CC2)CC(F)(F)F)cc1F)C1CCCCCC1]

N-((S)-1-cycloheptyl-2-((4-((S)-1-((cyclopropylmethyl)(2,2,2-trifluoroethyl)amino)-1-oxopropan-2-yl)-2-fluorophenyl)amino)-2-oxoethyl)-1-ethyl-1H-pyrazole-5-carboxamide (8). Prepared according to General Scheme C, employing methyl (S)-2-(4-amino-3-fluorophenyl)propanoate and (S)-2-((tert-butoxycarbonyl)amino)-2-cycloheptylacetic acid followed by 1-ethyl-1H-pyrazole-5-carboxylic acid and N-(cyclopropylmethyl)-2,2,2-trifluoroethan-1-amine hydrochloride. LCMS [M+H]⁺=594.3 m/z.

### Example 10: Synthesis of Compound 9

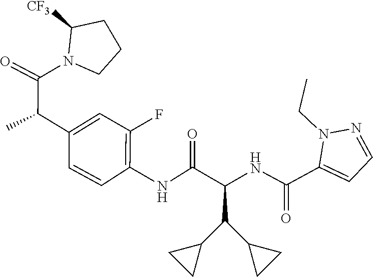 [SMILES: CCn1nccc1C(=O)N[C@H](C(=O)Nc1ccc([C@H](C)C(=O)N2CCC[C@@H]2C(F)(F)F)cc1F)C(C1CC1)C1CC1]

N-((S)-1,1-dicyclopropyl-3-((2-fluoro-4-((S)-1-oxo-1-((R)-2-(trifluoromethyl)pyrrolidin-1-yl)propan-2-yl)phenyl)amino)-3-oxopropan-2-yl)-1-ethyl-1H-pyrazole-5-carboxamide (9). Prepared according to General Scheme C, employing methyl (S)-2-(4-amino-3-fluorophenyl)propanoate and (S)-2-((tert-butoxycarbonyl)amino)-3,3-dicyclopropylpropanoic acid followed by 1-ethyl-1H-pyrazole-5-carboxylic acid and (R)-2-(trifluoromethyl)pyrrolidine. LCMS [M+H]⁺=578.2 m/z.

### Example 11: Synthesis of Compound 10

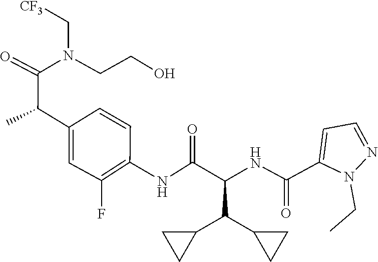 [SMILES: CCn1nccc1C(=O)N[C@H](C(=O)Nc1ccc([C@H](C)C(=O)N(CCO)CC(F)(F)F)cc1F)C(C1CC1)C1CC1]

N-((S)-1,1-dicyclopropyl-3-((2-fluoro-4-((S)-1-((2-hydroxyethyl)(2,2,2-trifluoroethyl)amino)-1-oxopropan-2-yl)phenyl)amino)-3-oxopropan-2-yl)-1-ethyl-1H-pyrazole-5-carboxamide (10). Prepared according to General Scheme C, employing methyl (S)-2-(4-amino-3-fluorophenyl)propanoate and (S)-2-((tert-butoxycarbonyl)amino)-3,3-dicyclopropylpropanoic acid followed by 1-ethyl-1H-pyrazole-5-carboxylic acid and 2-((2,2,2-trifluoroethyl)amino)ethan-1-ol.

LCMS [M+H]⁺=582.1 m/z.

### Example 12: Synthesis of Compound 11

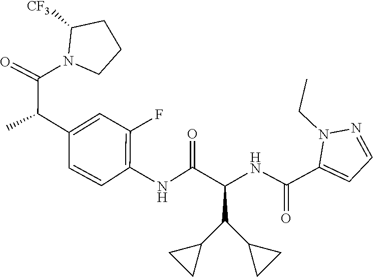 [SMILES: CCn1nccc1C(=O)N[C@H](C(=O)Nc1ccc([C@H](C)C(=O)N2CCC[C@H]2C(F)(F)F)cc1F)C(C1CC1)C1CC1]

N-((S)-1,1-dicyclopropyl-3-((2-fluoro-4-((S)-1-oxo-1-((S)-2-(trifluoromethyl)pyrrolidin-1-yl)propan-2-yl)phenyl)amino)-3-oxopropan-2-yl)-1-ethyl-1H-pyrazole-5-carboxamide (II). Prepared according to General Scheme C, employing methyl (S)-2-(4-amino-3-fluorophenyl)propanoate and (S)-2-((tert-butoxycarbonyl)amino)-3,3-dicyclopropylpropanoic acid followed by 1-ethyl-1H-pyrazole-5-carboxylic acid and (S)-2-(trifluoromethyl)pyrrolidine. LCMS [M+H]⁺=578.2 m/z.

### Example 13: Synthesis of Compound 12

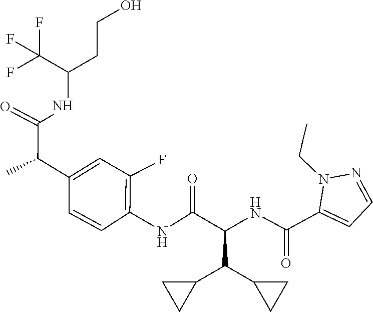 [SMILES: CCn1nccc1C(=O)N[C@H](C(=O)Nc1ccc([C@H](C)C(=O)NC(CCO)C(F)(F)F)cc1F)C(C1CC1)C1CC1]

N-((2S)-1,1-dicyclopropyl-3-((2-fluoro-4-((2S)-1-oxo-1-((1,1,1-trifluoro-4-hydroxybutan-2-yl)amino)propan-2-yl)phenyl)amino)-3-oxopropan-2-yl)-1-ethyl-1H-pyrazole-5-carboxanude (12). Prepared according to General Scheme C. employing methyl (S)-2-(4-amino-3-fluorophenyl)propanoate and (S)-2-((tert-butoxycarbonyl)amino)-3,3-dicyclopropylpropanoic acid followed by 1-ethyl-1H-pyrazole-5-carboxylic acid and 2-(trifluoromethyl)azetidine to afford N-((2S)-1-((4-((2S)-1-((4-chloro-1,1,1-trifluorobutan-2-yl)amino)-1-oxopropan-2-yl)-2-fluorophenyl)amino)-3,3-dicyclopropyl-1-oxopropan-2-yl)-1-ethyl-1H-pyrazole-5-carboxamide, which was then converted to the title compound by the following procedure: N-((2S)-1-((4-((2S)-1-((4-chloro-1,1,1-trifluorobutan-2-yl)amino)-1-oxopropan-2-yl)-2-fluorophenyl)amino)-3,3-dicyclopropyl-1-oxopropan-2-yl)-1-ethyl-1H-pyrazole-5-carboxamide (20.0 mg, 33.3 umol, 1.00 eq), DIEA (8.62 mg, 66.6 umol, 11.6 uL, 2.00 eq), and Na1 (499 ug, 3.33 umol, 0.100 eq) were added to a microwave vial and diluted with 1-PrOH (1.00 mL). The sealed tube was heated at 120° C. for 60 min under microwave. The reaction mixture was filtered, concentrated under reduced pressure, and purified by prep-HPLC (0-100% ACN in water with 0.225% formic acid) to afford N-((2S)-1,1-dicyclopropyl-3-((2-fluoro-4-((2S)-1-oxo-1-((1,1,1-trifluoro-4-hydroxybutan-2-yl)amino)propan-2-yl)phenyl)amino)-3-oxopropan-2-yl)-1-ethyl-1H-pyrazole-5-carboxamide (6.2 mg, 10 umol, 31% yield) as a white solid. LCMS [M+H]⁺=582.4 m/z.

### Example 14: Synthesis of Compound 13

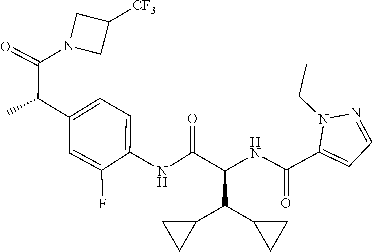 [SMILES: CCn1nccc1C(=O)N[C@H](C(=O)Nc1ccc([C@H](C)C(=O)N2CC(C(F)(F)F)C2)cc1F)C(C1CC1)C1CC1]

N-((S)-1,1-dicyclopropyl-3-((2-fluoro-4-((S)-1-oxo-1-((2,2,2-trifluoroethyl)amino)propan-2-yl)phenyl)amino)-3-oxopropan-2-yl)-1-ethyl-1H-pyrazole-5-carboxamide (13). Prepared according to General Scheme C, employing methyl (S)-2-(4-amino-3-fluorophenyl)propanoate and (S)-2-((tert-butoxycarbonyl)amino)-3,3-dicyclopropylpropanoic acid followed by 1-ethyl-1H-pyrazole-5-carboxylic acid and 3-(trifluoromethyl)azetidine hydrochloride. LCMS [M+H]⁺=564.3 m/z.

### Example 15: Synthesis of Compound 14

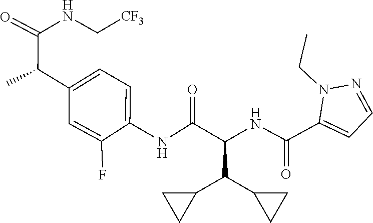 [SMILES: CCn1nccc1C(=O)N[C@H](C(=O)Nc1ccc([C@H](C)C(=O)NCC(F)(F)F)cc1F)C(C1CC1)C1CC1]

N-((S)-1,1-dicyclopropyl-3-((2-fluoro-4-((S)-1-oxo-1-(3-(trifluoromethyl)azetidin-1-yl)propan-2-yl)phenyl)amino)-3-oxopropan-2-yl)-1-ethyl-1H-pyrazole-5-carboxamide (14). Prepared according to General Scheme C, employing methyl (S)-2-(4-amino-3-fluorophenyl)propanoate and (S)-2-((tert-butoxycarbonyl)amino)-3,3-dicyclopropylpropanoic acid followed by 1-ethyl-1H-pyrazole-5-carboxylic acid and 2,2,2-trifluoroethan-1-amine hydrochloride.

LCMS [M+H]⁺=538.3 m/z.

### Example 16: Synthesis of Compound 15

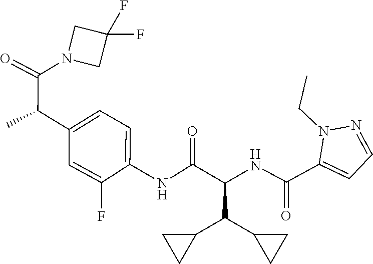 [SMILES: CCn1nccc1C(=O)N[C@H](C(=O)Nc1ccc([C@H](C)C(=O)N2CC(F)(F)C2)cc1F)C(C1CC1)C1CC1]

N-((S)-1,1-dicyclopropyl-3-((4-((S)-1-(3,3-difluoroazetidin-1-yl)-1-oxopropan-2-yl)-2-fluorophenyl)amino)-3-oxopropan-2-yl)-1-ethyl-1H-pyrazole-5-carboxamide (15). Prepared according to General Scheme C, employing methyl (S)-2-(4-amino-3-fluorophenyl)propanoate and (S)-2-((tert-butoxycarbonyl)amino)-3,3-dicyclopropylpropanoic acid followed by 1-ethyl-1H-pyrazole-5-carboxylic acid and 3,3-difluoroazetidine hydrochloride. LCMS [M+H]⁺=532.1 m/z.

### Example 17: Synthesis of Compound 16

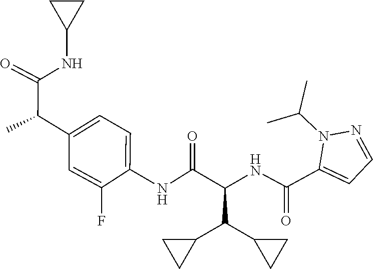 [SMILES: CC(C)n1nccc1C(=O)N[C@H](C(=O)Nc1ccc([C@H](C)C(=O)NC2CC2)cc1F)C(C1CC1)C1CC1]

N-((S)-1,1-dicyclopropyl-3-((4-((S)-1-(cyclopropylamino)-1-oxopropan-2-yl)-2-fluorophenyl)amino)-3-oxopropan-2-yl)-1-isopropyl-1H-pyrazole-5-carboxamide (16). Prepared according to General Scheme C, employing methyl (S)-2-(4-amino-3-fluorophenyl)propanoate and (S)-2-((tert-butoxycarbonyl)amino)-3,3-dicyclopropylpropanoic acid followed by 1-isopropyl-1H-pyrazole-5-carboxylic acid and cyclopropanamine. LCMS [M+H]⁺=510.2 m/z.

### Example 18: Synthesis of Compound 17

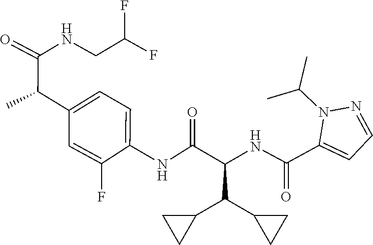 [SMILES: CC(C)n1nccc1C(=O)N[C@H](C(=O)Nc1ccc([C@H](C)C(=O)NCC(F)F)cc1F)C(C1CC1)C1CC1]

N-((S)-1,1-dicyclopropyl-3-((4-((S)-1-((2,2-difluoroethyl)amino)-1-oxopropan-2-yl)-2-fluorophenyl)amino)-3-oxopropan-2-yl)-1-isopropyl-1H-pyrazole-5-carboxanide (17). Prepared according to General Scheme C, employing methyl (S)-2-(4-amino-3-fluorophenyl)propanoate and (S)-2-((tert-butoxycarbonyl)amino)-3,3-dicyclopropylpropanoic acid followed by 1-isopropyl-1H-pyrazole-5-carboxylic acid and 2,2-difluoroethan-1-amine. LCMS [M+H]⁺=534.3 m/z.

### Example 19: Synthesis of Compound 18

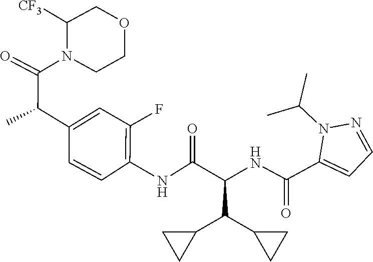 [SMILES: CC(C)n1nccc1C(=O)N[C@H](C(=O)Nc1ccc([C@H](C)C(=O)N2CCOCC2C(F)(F)F)cc1F)C(C1CC1)C1CC1]

N-((2S)-1,1-dicyclopropyl-3-((2-fluoro-4-((2S)-1-oxo-1-(3-(trifluoromethyl)morpholino)propan-2-yl)phenyl)amino)-3-oxopropan-2-yl)-1-isopropyl-1H-pyrazole-5-carboxamide (18). Prepared according to General Scheme C. employing methyl (S)-2-(4-amino-3-fluorophenyl)propanoate and (S)-2-((tert-butoxycarbonyl)amino)-3,3-dicyclopropylpropanoic acid followed by 1-isopropyl-1H-pyrazole-5-carboxylic acid and 3-(trifluoromethyl)morpholine hydrochloride. The title compound was isolated as the first eluting, single stereoisomer by two tandem chiral SFC purifications (First: column: Phenomenex-Cellulose-2 (250 mm×30 mm, 10 um): mobile phase: 30% [0.1% NH₃H₂O in methanol]: Second: column: DAICEL CHIRALPAK AD (250 mm×30 mm, 10 um); mobile phase: 25% 10.1% NH₃H₂O in IPA]). LCMS [M+H]⁺=608.3 m/z.

### Example 20: Synthesis of Compound 19

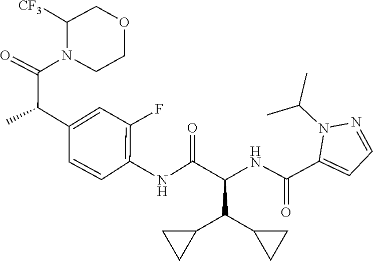 [SMILES: CC(C)n1nccc1C(=O)N[C@H](C(=O)Nc1ccc([C@H](C)C(=O)N2CCOCC2C(F)(F)F)cc1F)C(C1CC1)C1CC1]

N-((2S)-1,1-dicyclopropyl-3-((2-fluoro-4-((2S)-1-oxo-1-(3-(trifluoromethyl)morpholino)propan-2-yl)phenyl)amino)-3-oxopropan-2-yl)-isopropyl-1H-pyrazole-5-carboxamide (19). Prepared according to General Scheme C. employing methyl (S)-2-(4-amino-3-fluorophenyl)propanoate and (S)-2-((tert-butoyvcarbonyl)amino)-3,3-dicyclopropylpropanoic acid followed by 1-isopropyl-1H-pyrazole-5-carboxylic acid and 3-(trifluoromethyl)morpholine hydrochloride. The title compound was isolated as the second eluting, single stereoisomer by two tandem chiral SFC purifications (First: column: Phenomenex-Cellulose-2 (250 mm×30 mm, 10 um); mobile phase: 30% 10.10% NH₃H₂O in methanol]: Second: column: DAICEL CHIRALPAK AD (250 mm×30 mm, 10 um); mobile phase: 25% 10.1% NH₃H₂O in IPA]). LCMS [M+H]⁺=608.3 m/z.

### Example 21: Synthesis of Compound 20

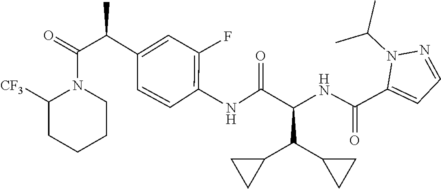 [SMILES: CC(C)n1nccc1C(=O)N[C@H](C(=O)Nc1ccc([C@H](C)C(=O)N2CCCCC2C(F)(F)F)cc1F)C(C1CC1)C1CC1]

N-((2S)-1,1-dicyclopropyl-3-((2-fluoro-4-((2S)-1-oxo-1-(2-(trifluoromethyl)piperidin-1-yl)propan-2-yl)phenyl)amino)-3-oxopropan-2-yl)-1-isopropyl-1H-pyrazole-5-carboxamide (20). Prepared according to General Scheme C, employing methyl (S)-2-(4-amino-3-fluorophenyl)propanoate and (S)-2-((tert-butoxycarbonyl)amino)-3,3-dicyclopropylpropanoic acid followed by 1-isopropyl-1H-pyrazole-5-carboxylic acid and 2-(trifluoromethyl)piperidine. The title compound was isolated as the first eluting, single stereoisomer by chiral SFC purification (column: DAICEL CHIRALPAK AD (250 mm×30 mm, 10 um): mobile phase: 20% [0.1% NH₃H₂O in IPA|). LCMS [M+H]⁺=606.4 mi.

### Example 22: Synthesis of Compound 21

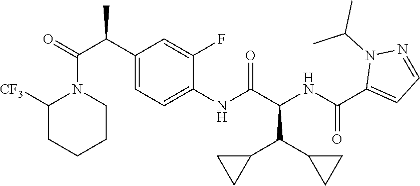 [SMILES: CC(C)n1nccc1C(=O)N[C@H](C(=O)Nc1ccc([C@H](C)C(=O)N2CCCCC2C(F)(F)F)cc1F)C(C1CC1)C1CC1]

N-((2S)-1,1-dicyclopropyl-3-((2-fluoro-4-((2S)-1-oxo-1-(2-(trifluoromethyl)piperidin-1-yl)propan-2-yl)phenyl)amino)-3-oxopropan-2-yl)-1-isopropyl-1H-pyrazole-5-carboxamide (21). Prepared according to General Scheme C, employing methyl (S)-2-(4-amino-3-fluorophenyl)propanoate and (S)-2-((tert-butoxycarbonyl)amino)-3,3-dicyclopropylpropanoic acid followed by 1-isopropyl-1H-pyrazole-5-carboxylic acid and (2-(trifluoromethyl)piperidine. The title compound was isolated as the second eluting, single stereoisomer by chiral SFC purification (column: DAICEL CHIRALPAK AD (250 mm×30 mm, 10 um); mobile phase: 20% [0.1% NH₃H₂O in IPA]). LCMS [M+H]⁺=606.3 m/z.

### Example 23: Synthesis of Compound 22

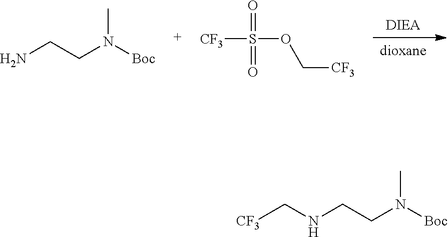 [SMILES: CN(CCN)C(=O)OC(C)(C)C ; O=S(=O)(OCC(F)(F)F)C(F)(F)F ; "DIEA" ; "dioxane" ; CN(CCNCC(F)(F)F)C(=O)OC(C)(C)C]

Tert-butyl methyl(2-((2,2,2-trifluoroethyl)amino)ethyl)carbamate. To a solution of tert-butyl (2-aminoethyl)(methyl)carbamate (1.00 g, 5.74 mmol, 1.03 mL, 1.00 eq) in dioxane (10.0 mL) was added DIEA (1.11 g, 8.61 mmol, 1.50 mL, 1.50 eq) and 2,2,2-trifluoroethyl trifluoromethanesulfonate (1.63 g, 6.89 mmol, 1.20 eq). The mixture was stirred at 100° C. for 12 h. The residue was diluted with H₂O and extracted with EtOAc. The combined organic layers were washed with brine, dried over Na₂SO₄, filtered, concentrated under reduced pressure, and purified by column chromatography (SiO₂. Petroleum ether: Ethyl acetate=100:1 to 1:1) to afford tert-butyl methyl(2-((2,2,2-trifluoroethyl)amino)ethyl)carbamate (830 mg, 3.24 mmol, 56.4% yield) as a white solid. LCMS [M+H]⁺=257.1 m/z.

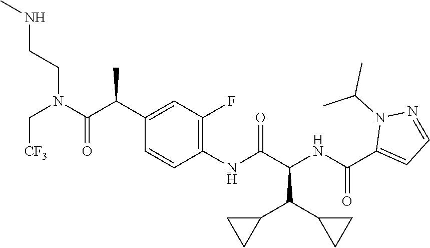 [SMILES: CNCCN(CC(F)(F)F)C(=O)[C@@H](C)c1ccc(NC(=O)[C@@H](NC(=O)c2ccnn2C(C)C)C(C2CC2)C2CC2)c(F)c1]

N-((S)-1,1-dicyclopropyl-3-((2-fluoro-4-((S)-1-((2-(methylamino)ethyl)(2,2,2-trifluoroethyl)amino)-1-oxopropan-2-yl)phenyl)amino)-3-oxopropan-2-yl)-1-isopropyl-1H-pyrazole-5-carboxamide (22). tert-butyl (2-((S)-2-(4-((S)-3,3-dicyclopropyl-2-(1-isopropyl-1H-pyrazole-5-carboxamido)propanamido)-3-fluorophenyl)-N-(2,2,2-trifluoroethyl)propanamido)ethyl)(methyl)carbamate was prepared according to General Scheme C. employing methyl (S)-2-(4-amino-3-fluorophenyl)propanoate and (S)-2-((tert-butoxycarbonyl)amino)-3,3-dicyclopropylpropanoic acid followed by 1-isopropyl-1H-pyrazole-5-carboxylic acid and tert-butyl methyl(2-((2,2,2-trifluoroethyl)amino)ethyl)carbamate. The title compound was prepared according to General Procedure C, employing tert-butyl (2-((S)-2-(4-((S)-3,3-dicyclopropyl-2-(1-isopropyl-1H-pyrazole-5-carboxamido)propanamido)-3-fluorophenyl)-N-(2,2,2-trifluoroethyl)propanamido)ethyl)methyl)carbamate. LCMS [M+H]⁺=609.3 m/z.

### Example 24: Synthesis of Compound 23

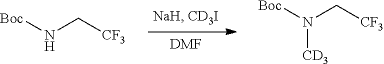

Tert-butyl (methyl-d3)(2,2,2-trifluoroethyl)carbamate. To a solution of tert-butyl (2,2,2-trifluoroethyl)carbamate (500 mg, 2.51 mmol, 1.00 eq) in DMF (3.00 mL) was added NaH (150 mg, 3.75 mmol, 60% purity, 1.49 eq) at 0° C. The mixture was stirred at 0° C. for 0.2 h. CD₃I (500 mg, 3.52 mmol, 215 μL, 1.40 eq) was added to the reaction mixture, and the mixture was stirred atRT for 2 h. The reaction mixture was diluted with H₂O and extracted with DCM. The combined organic layers were washed with H₂O followed by brine, dried over Na₂SO₄, filtered, and concentrated under reduced pressure to afford tert-butyl (methyl-d3)(2,2,2-trifluoroethyl)carbamate (300 mg, 1.39 mmol, 55.3% yield) as yellow oil. LCMS [M+H]⁺=161.1 m/z.

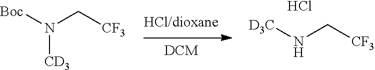

2,2,2-trifluoro-N-(methyl-d3)ethan-1-amine. To a solution of tert-butyl (methyl-d3)(2,2,2-trifluoroethyl)carbamate (300 mg, 1.39 mmol, 1.00 eq) in DCM (2.00 mL) was added HCl/dioxane (4 M, 2.00 mL, 5.77 eq) at 0° C. The mixture was stirred at RT for 2 h. The reaction mixture was concentrated under reduced pressure to afford 2,2,2-trifluoro-N-(methyl-d3)ethan-1-amine (2(X) mg, 1.31 mmol, 94.4% yield, HCl) as yellow oil.

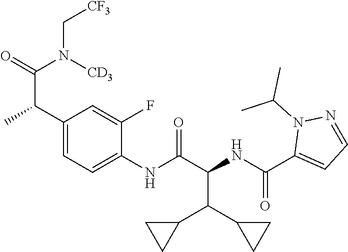 [SMILES: [2H]C([2H])([2H])N(CC(F)(F)F)C(=O)[C@@H](C)c1ccc(NC(=O)[C@@H](NC(=O)c2ccnn2C(C)C)C(C2CC2)C2CC2)c(F)c1]

N-((S)-1,1-dicyclopropyl-3-((2-fluoro-4-((S)-1-((methyl-d3)(2,2,2-trifluoroethyl)amino)-1-oxopropan-2-yl)phenyl)amino)-3-oxopropan-2-yl)-1-isopropyl-1H-pyrazole-5-carboxamide (23). Prepared according to General Scheme C, employing methyl (S)-2-(4-amino-3-fluorophenyl)propanoate and (S)-2-((tert-butoxycarbonyl)amino)-3,3-dicyclopropylpropanoic acid followed by 1-isopropyl-1H-pyrazole-5-carboxylic acid and 2,2,2-trifluoro-N-(methyl-dL)ethan-1-amine. LCMS [M+H]⁺=569.3 m/z.

### Example 25: Synthesis of Compound 24

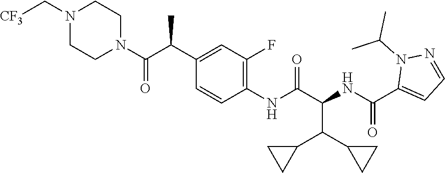 [SMILES: CC(C)n1nccc1C(=O)N[C@H](C(=O)Nc1ccc([C@H](C)C(=O)N2CCN(CC(F)(F)F)CC2)cc1F)C(C1CC1)C1CC1]

N-((S)-1.1-dicyclopropyl-3-((2-fluoro-4-((S)-1-oxo-1-(4-(2,2,2-trifluoroethyl)piperazin-1-yl)propan-2-yl)phenyl)amino)-3-oxopropan-2-yl)-1-isopropyl-1H-pyrazole-5-carboxamide (24). Prepared according to General Scheme C, employing methyl (S)-2-(4-amino-3-fluorophenyl)propanoate and (S)-2-((tert-butoxycarbonyl)amino)-3,3-dicyclopropylpropanoic acid followed by 1-isopropyl-1H-pyrazole-5-carboxylic acid and 1-(2,2,2-trifluoroethyl)piperazine.

LCMS [M+H]⁺=621.2 m/z.

### Example 26: Synthesis of Compound 25

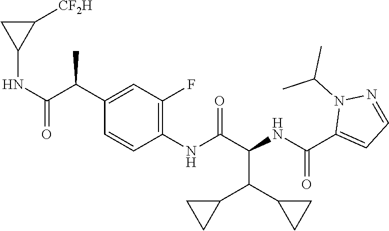 [SMILES: CC(C)n1nccc1C(=O)N[C@H](C(=O)Nc1ccc([C@H](C)C(=O)NC2CC2C(F)F)cc1F)C(C1CC1)C1CC1]

N-((2S)-1.1-dicyclopropyl-3-((4-((2S)-1-((2-(difluoromethyl)cyclopropyl)amino)-1-oxopropan-2-yl)-2-fluorophenyl)amino)-3-oxopropan-2-yl)-1-isopropyl-1H-pyrazole-5-carboxamide (25). Prepared according to General Scheme C. employing methyl (S)-2-(4-amino-3-fluorophenyl)propanoate and (S)-2-((tert-butoxycarbonyl)amino)-3,3-dicyclopropylpropanoic acid followed by 1-isopropyl-1H-pyrazole-5-carboxylic acid and 2-(difluoromethyl)cyclopropan-1-amine hydrochloride. LCMS [M+H]⁺=560.2 m/z.

### Example 27: Synthesis of Compound 26

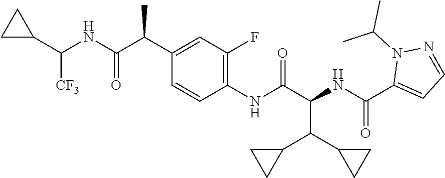 [SMILES: CC(C)n1nccc1C(=O)N[C@H](C(=O)Nc1ccc([C@H](C)C(=O)NC(C2CC2)C(F)(F)F)cc1F)C(C1CC1)C1CC1]

N-((2S)-1,1-dicyclopropyl-3-((4-((2S)-1-((1-cyclopropyl-2,2,2-trifluoroethyl)amino)-1-oxopropan-2-yl)-2-fluorophenyl)amino)-3-oxopropan-2-yl)-1-isopropyl-1H-pyrazole-5-carboxamide (26). Prepared according to General Scheme C, employing methyl (S)-2-(4-amino-3-fluorophenyl)propanoate and (S)-2-((tert-butoxycarbonyl)amino)-3,3-dicyclopropylpropanoic acid followed by 1-isopropyl-1H-pyrazole-5-carboxylic acid and 1-cyclopropyl-2,2,2-trifluoroethan-1-amine. LCMS [M+H]⁺=592.2 m/z.

### Example 28: Synthesis of Compound 27

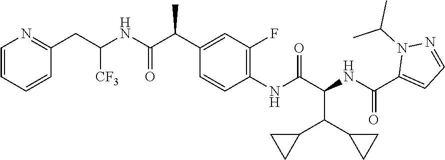 [SMILES: CC(C)n1nccc1C(=O)N[C@H](C(=O)Nc1ccc([C@H](C)C(=O)NC(Cc2ccccn2)C(F)(F)F)cc1F)C(C1CC1)C1CC1]

N-((2S)-1,1-dicyclopropyl-3-((2-fluoro-4-((2S)-1-oxo-1-((1,1,1-trifluoro-3-(pyridin-2-yl)propan-2-yl)amino)propan-2-yl)phenyl)amino)-3-oxopropan-2-yl)-1-isopropyl-1H-pyrazole-5-carboxamide (27). Prepared according to General Scheme C. employing methyl (S)-2-(4-amino-3-fluorophenyl)propanoate and (S)-2-((tert-butoxycarbonyl)amino)-3,3-dicyclopropylpropanoic acid followed by 1-isopropyl-1H-pyrazole-5-carboxylic acid and 1,1,1-trifluoro-3-(pyridin-2-yl)propan-2-amine. LCMS [M+H]⁺=643.2m/z.

### Example 29: Synthesis of Compound 28

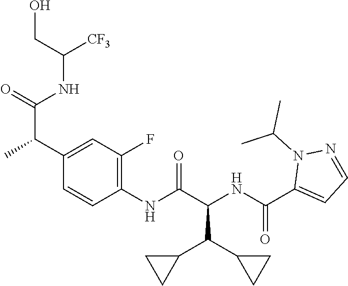 [SMILES: CC(C)n1nccc1C(=O)N[C@H](C(=O)Nc1ccc([C@H](C)C(=O)NC(CO)C(F)(F)F)cc1F)C(C1CC1)C1CC1]

N-((2S)-1.1-dicyclopropyl-3-((2-fluoro-4-((2S)-1-oxo-1-((1,1,1-trifluoro-3-hydroxypropan-2-yl)amino)propan-2-yl)phenyl)amino)-3-oxopropan-2-yl)-1-isopropy1-1H-pyrazole-5-carboxamide (28). Prepared according to General Scheme C. employing methyl (S)-2-(4-amino-3-fluorophenyl)propanoate and (S)-2-((tert-butoxycarbonyl)amino)-3,3-dicyclopropylpropanoic acid followed by 1-isopropyl-1H-pyrazole-5-carboxylic acid and 2-amino-3,3,3-trifluoropropan-1-ol hydrochloride. The title compound was isolated as the first eluting, single stereoisomer by chiral SFC purification (column: Regis Whelk-01 (250 mm×30 mm, 10 um); mobile phase: 25% [0.1% NH₃H₂O in MeOH]). LCMS [M+H]⁺=582.3 m/z.

### Example 30: Synthesis of Compound 29

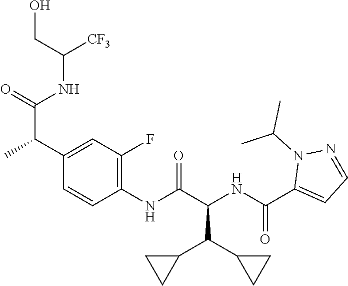 [SMILES: CC(C)n1nccc1C(=O)N[C@H](C(=O)Nc1ccc([C@H](C)C(=O)NC(CO)C(F)(F)F)cc1F)C(C1CC1)C1CC1]

N-((2S)-1,1-dicyclopropyl-3-((2-fluoro-4-((2S)-1-oxo-1-((1,1,1-trifluoro-3-hydroxypropan-2-yl)amino)propan-2-yl)phenyl)amino)-3-oxopropan-2-yl)-1-isopropyl-1H-pyrazole-5-carboxamide (29). Prepared according to General Scheme C, employing methyl (S)-2-(4-amino-3-fluorophenyl)propanoate and (S)-2-((tert-butoxycarbonyl)amino)-3,3-dicyclopropylpropanoic acid followed by 1-isopropyl-1H-pyrazole-5-carboxylic acid and 2-amino-3,3,3-trifluoropropan-1-ol hydrochloride. The title compound was isolated as the second eluting, single stereoisomer by chiral SFC purification (column: Regis Whelk-01 (250 mm×30 mm, 10 um); mobile phase: 25% [0.1% NH₃H₂O in MeOH]). LCMS [M+H]⁺=582.3 m/z.

### Example 31: Synthesis of Compound 30

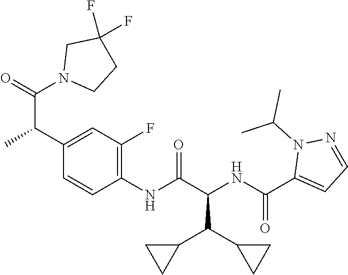 [SMILES: CC(C)n1nccc1C(=O)N[C@H](C(=O)Nc1ccc([C@H](C)C(=O)N2CCC(F)(F)C2)cc1F)C(C1CC1)C1CC1]

N-((S)-1,1-dicyclopropyl-3-((4-((S)-143,3-difluoropyrrolidin-1-yl)-1-oxopropan-2-yl)-2-fluorophenyl)amino)-3-oxopropan-2-yl)-1-isopropyl-1H-pyrazole-5-carboxanide (30). Prepared according to General Scheme C, employing methyl (S)-2-(4-amino-3-fluorophenyl)propanoate and (S)-2-((tert-butoxycarbonyl)amino)-3,3-dicyclopropylpropanoic acid followed by 1-isopropyl-1H-pyrazole-5-carboxylic acid and 3,3-difluoropyrrolidine. LCMS [M+H]⁺=560.2 m/z.

### Example 32: Synthesis of Compound 31

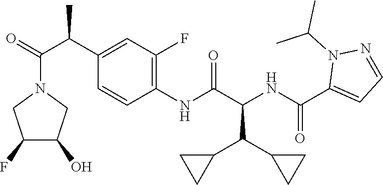 [SMILES: CC(C)n1nccc1C(=O)N[C@H](C(=O)Nc1ccc([C@H](C)C(=O)N2C[C@@H](O)[C@@H](F)C2)cc1F)C(C1CC1)C1CC1]

N-((S)-1.1-dicyclopropyl-3-((2-fluoro-4-((S)-1-((3S,4R)-3-fluoro-4-hydroxypyrrolidin-1-yl)-1-oxopropan-2-yl)phenyl)amino)-3-oxopropan-2-yl)-1-isopropyl-1H-pyrazole-5-carboxamide (31). Prepared according to General Scheme C, employing methyl (S)-2-(4-amino-3-fluorophenyl)propanoate and (S)-2-((tert-butoxycarbonyl)amino)-3,3-dicyclopropylpropanoic acid followed by 1-isopropyl-1H-pyrazole-5-carboxylic acid and (3R,4S)-4-fluoropyrrolidin-3-ol hydrochloride. LCMS [M+H]⁺=558.2 m/z.

### Example 33: Synthesis of Compound 32

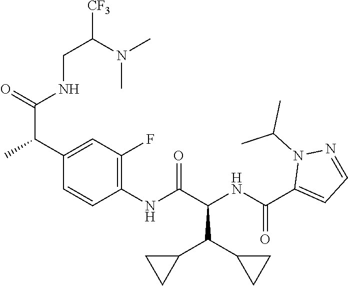 [SMILES: CC(C)n1nccc1C(=O)N[C@H](C(=O)Nc1ccc([C@H](C)C(=O)NCC(N(C)C)C(F)(F)F)cc1F)C(C1CC1)C1CC1]

N-((2S)-1.1-dicyclopropyl-3-((4-((2S)-1-((2-(dimethylamino)-3,3,3-trifluoropropyl)amino)-1-oxopropan-2-yl)-2-fluorophenyl)amino)-3-oxopropan-2-yl)-1-isopropyl-1H-pyrazole-5-carboxamide (32). Prepared according to General Scheme C. employing methyl (S)-2-(4-amino-3-fluorophenyl)propanoate and (S)-2-((tert-butoxycarbonyl)amino)-3,3-dicyclopropylpropanoic acid followed by 1-isopropyl-1H-pyrazole-5-carboxylic acid and 3,3,3-trifluoro-N2,N2-dimethyl-propane-1,2-diamine dihydrochloride. LCMS [M+H]⁺=609.2 m/z.

### Example 34: Synthesis of Compound 33

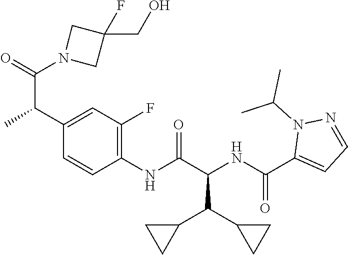 [SMILES: CC(C)n1nccc1C(=O)N[C@H](C(=O)Nc1ccc([C@H](C)C(=O)N2CC(F)(CO)C2)cc1F)C(C1CC1)C1CC1]

N-((S)-1,1-dicyclopropyl-3-((2-fluoro-4-((S)-1-(3-fluoro-3-(hydroxymethyl)azetidin-1-yl)-1-oxopropan-2-yl)phenyl)amino)-3-oxopropan-2-yl)-1-isopropyl-1H-pyrazole-5-carboxamide (33). Prepared according to General Scheme C, employing methyl (S)-2-(4-amino-3-fluorophenyl)propanoate and (S)-2-((tert-butoxycarbonyl)amino)-3,3-dicyclopropylpropanoic acid followed by 1-isopropyl-1H-pyrazole-5-carboxylic acid and (3-fluoroazetidin-3-yl)methanol.

LCMS [M+H]⁺=558.2 m/z.

### Example 35: Synthesis of Compound 34

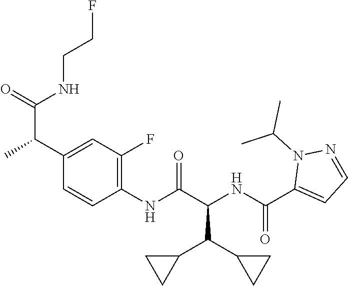 [SMILES: CC(C)n1nccc1C(=O)N[C@H](C(=O)Nc1ccc([C@H](C)C(=O)NCCF)cc1F)C(C1CC1)C1CC1]

N-((S)-1,1-dicyclopropyl-3-((2-fluoro-4-((S)-1-((2-fluoroethyl)amino)-1-oxopropan-2-yl)phenyl)amino)-3-oxopropan-2-yl)-1-isopropyl-1H-pyrazole-5-carboxamide (34). Prepared according to General Scheme C. employing methyl (S)-2-(4-amino-3-fluorophenyl)propanoate and (S)-2-((tert-butoxycarbonyl)amino)-3,3-dicyclopropylpropanoic acid followed by 1-isopropyl-1H-pyrazole-5-carboxylic acid and 2-fluoroethanamine hydrochloride. LCMS [M+H]⁺=516.2 m/z.

### Example 36: Synthesis of Compound 35

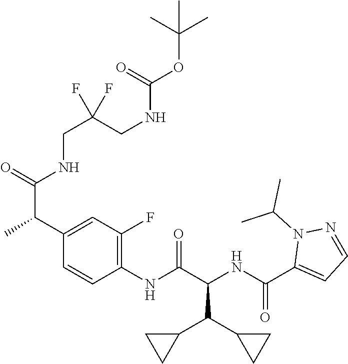 [SMILES: CC(C)n1nccc1C(=O)N[C@H](C(=O)Nc1ccc([C@H](C)C(=O)NCC(F)(F)CNC(=O)OC(C)(C)C)cc1F)C(C1CC1)C1CC1]

tert-butyl (3-((S)-2-(4-((S)-3,3-dicyclopropyl-2-(1-isopropyl-1H-pyrazole-5-carboxamido)propanamido)-3-fluorophenyl)propanamido)-2,2-difluoropropyl)carbamate (35). Prepared according to General Scheme C, employing methyl (S)-2-(4-amino-3-fluorophenyl)propanoate and (S)-2-((tert-butoxycarbonyl)amino)-3,3-dicyclopropylpropanoic acid followed by 1-isopropyl-1H-pyrazole-5-carboxylic acid and tert-butyl N-(3-amino-2,2-difluoro-propyl)carbamate. LCMS [M+H]⁺=628.2 m/z.

### Example 37: Synthesis of Compound 36

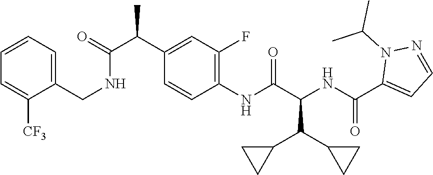 [SMILES: CC(C)n1nccc1C(=O)N[C@H](C(=O)Nc1ccc([C@H](C)C(=O)NCc2ccccc2C(F)(F)F)cc1F)C(C1CC1)C1CC1]

N-((S)-1.1-dicyclopropyl-3-((2-fluoro-4-((S)-1-oxo-1-((2-(trifluoromethyl)benzyl)amino)propan-2-yl)phenyl)amino)-3-oxopropan-2-yl)-1-isopropyl-1H-pyrazole-5-carboxamide (36). Prepared according to General Scheme C, employing methyl (S)-2-(4-amino-3-fluorophenyl)propanoate and (S)-2-((tert-butoxycarbonyl)amino)-3,3-dicyclopropylpropanoic acid followed by 1-isopropyl-1H-pyrazole-5-carboxylic acid and 12-(trifluoromethyl)phenyl]methanamine. LCMS [M+H]⁺=663.3 m.

### Example 38: Synthesis of Compound 37

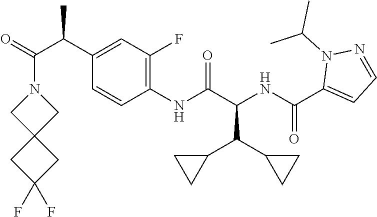 [SMILES: CC(C)n1nccc1C(=O)N[C@H](C(=O)Nc1ccc([C@H](C)C(=O)N2CC3(C2)CC(F)(F)C3)cc1F)C(C1CC1)C1CC1]

N-((S)-1.1-dicyclopropyl-3-((4-((S)-1-(6,6-difluoro-2-azaspiro[3.3|heptan-2-yl)-1-oxopropan-2-yl)-2-fluorophenyl)amino)-3-oxopropan-2-yl)-1-isopropyl-1H-pyrazole-5-carboxamide (37). Prepared according to General Scheme C, employing methyl (S)-2-(4-amino-3-fluorophenyl)propanoate and (S)-2-((tert-butoxycarbonyl)amino)-3,3-dicyclopropylpropanoic acid followed by 1-isopropyl-1H-pyrazole-5-carboxylic acid and 6,6-difluoro-2-azaspirol3.3]heptane hydrochloride. LCMS [M+H]⁺=586.2 m/z.

### Example 39: Synthesis of Compound 38

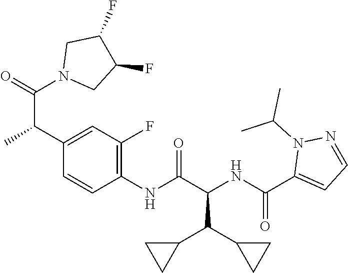 [SMILES: CC(C)n1nccc1C(=O)N[C@H](C(=O)Nc1ccc([C@H](C)C(=O)N2C[C@H](F)[C@@H](F)C2)cc1F)C(C1CC1)C1CC1]

N-((S)-1,1-dicyclopropyl-3-((4-((S)-1-((3S,4S)-3,4-difluoropyrrolidin-1-yl)-1-oxopropan-2-yl)-2-fluorophenyl)amino)-3-oxopropan-2-yl)-1-isopropyl-1H-pyrazole-5-carboxamide (38). Prepared according to General Scheme C, employing methyl (S)-2-(4-amino-3-fluorophenyl)propanoate and (S)-2-((tert-butoxycarbonyl)amino)-3,3-dicyclopropylpropanoic acid followed by 1-isopropyl-1H-pyrazole-5-carboxylic acid and (3S,4S)-3,4-difluoropyrrolidine hydrochloride. LCMS [M+H]⁺=560.2 m/z.

### Example 40: Synthesis of Compound 39

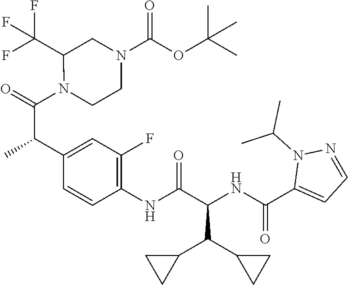 [SMILES: CC(C)n1nccc1C(=O)N[C@H](C(=O)Nc1ccc([C@H](C)C(=O)N2CCN(C(=O)OC(C)(C)C)CC2C(F)(F)F)cc1F)C(C1CC1)C1CC1]

tert-butyl 4-((S)-2-(4-((S)-3,3-dicyclopropyl-2-(1-isopropyl-1H-pyrazole-5-carboxamido)propanamido)-3-fluorophenyl)propanoyl)-3-(trifluoromethyl)piperazine-1-carboxylate (39). Prepared according to General Scheme C, employing methyl (S)-2-(4-amino-3-fluorophenyl)propanoate and (S)-2-((tert-butoxycarbonyl)amino)-3,3-dicyclopropylpropanoic acid followed by 1-isopropyl-1H-pyrazole-5-carboxylic acid and tert-butyl 3-(trifluoromethyl)piperazine-1-carboxylate. LCMS [M+H]⁺=707.2 m/z.

### Example 41: Synthesis of Compound 40

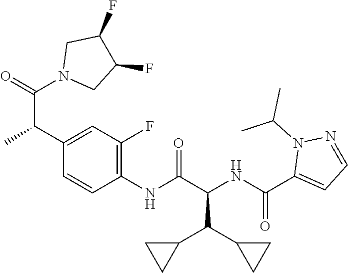 [SMILES: CC(C)n1nccc1C(=O)N[C@H](C(=O)Nc1ccc([C@H](C)C(=O)N2C[C@@H](F)[C@@H](F)C2)cc1F)C(C1CC1)C1CC1]

N-((S)-1,1-dicyclopropyl-3-((4-((S)-1-((3S,4R)-3,4-difluoropyrrolidin-1-yl)-1-oxopropan-2-yl)-2-fluorophenyl)amino)-3-oxopropan-2-yl)-1-isopropyl-1H-pyrazole-5-carboxamide (40). Prepared according to General Scheme C, employing methyl (S)-2-(4-amino-3-fluorophenyl)propanoate and (S)-2-((tert-butoxycarbonyl)amino)-3,3-dicyclopropylpropanoic acid followed by 1-isopropyl-1H-pyrazole-5-carboxylic acid and (3S.4R)-3,4-difluoropyrrolidine hydrochloride. LCMS [M+H]⁺=560.2 m,-.

### Example 42: Synthesis of Compound 41

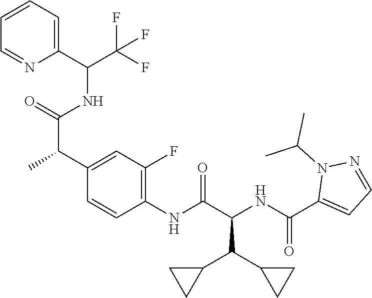 [SMILES: CC(C)n1nccc1C(=O)N[C@H](C(=O)Nc1ccc([C@H](C)C(=O)NC(c2ccccn2)C(F)(F)F)cc1F)C(C1CC1)C1CC1]

N-((2S)-1,1-dicyclopropyl-3-((2-fluoro-4-((2S)-1-oxo-1-((2,2,2-trifluoro-1-(pyridin-2-yl)ethyl)amino)propan-2-yl)phenyl)amino)-3-oxopropan-2-yl)-1-isopropyl-1 H-pyrazole-5-carboxamide (41). Prepared according to General Scheme C. employing methyl (S)-2-(4-amino-3-fluorophenyl)propanoate and (S)-2-((tert-butoxycarbonyl)amino)-3,3-dicyclopropylpropanoic acid followed by 1-isopropyl-1H-pyrazole-5-carboxylic acid and 2,2,2-trifluoro-1-(2-pyridyl)ethanamine dihydrochloride. LCMS [M+H]⁺=629.2 m/z.

### Example 43: Synthesis of Compound 42

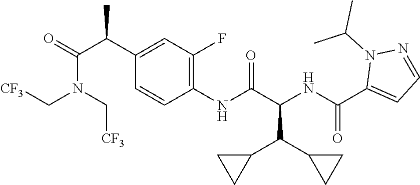 [SMILES: CC(C)n1nccc1C(=O)N[C@H](C(=O)Nc1ccc([C@H](C)C(=O)N(CC(F)(F)F)CC(F)(F)F)cc1F)C(C1CC1)C1CC1]

N-((S)-1-((4-((S)-1-(bis(2,2,2-trifluoroethyl)amino)-1-oxopropan-2-yl)-2-fluorophenyl)amino)-3,3-dicyclopropyl-1-oxopropan-2-yl)-1-isopropyl-1H-pyrazole-5-carboxamide (42). Prepared according to General Scheme C, employing methyl (S)-2-(4-amino-3-fluorophenyl)propanoate and (S)-2-((tert-butoxycarbonyl)amino)-3.3-dicyclopropylpropanoic acid followed by 1-isopropyl-1H-pyrazole-5-carboxylic acid and 2,2,2-trifluoro-N-(2,2,2-trifluoroethyl)ethanamine. LCMS [M+H]⁺=634.2 m/z.

### Example 44: Synthesis of Compound 43

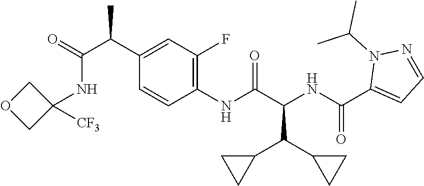 [SMILES: CC(C)n1nccc1C(=O)N[C@H](C(=O)Nc1ccc([C@H](C)C(=O)NC2(C(F)(F)F)COC2)cc1F)C(C1CC1)C1CC1]

N-((S)-1.1-dicyclopropyl-3-((2-fluoro-4-((S)-1-oxo-1-((3-(trifluoromethyl)oxetan-3-yl)amino)propan-2-yl)phenyl)amino)-3-oxopropan-2-yl)-1-isopropyl-1H-pyrazole-5-carboxamide (43). Prepared according to General Scheme C, employing methyl (S)-2-(4-amino-3-fluorophenyl)propanoate and (S)-2-((tert-butoxycarbonyl)amino)-3,3-dicyclopropylpropanoic acid followed by 1-isopropyl-1H-pyrazole-5-carboxylic acid and 3-(trifluoromethyl)oxetan-3-amine hydrochloride. LCMS [M+H]⁺=594.2 m/z.

### Example 45: Synthesis of Compound 44

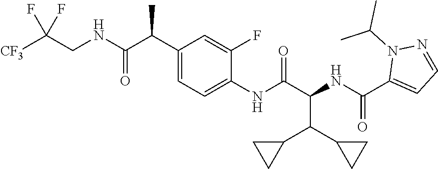 [SMILES: CC(C)n1nccc1C(=O)N[C@H](C(=O)Nc1ccc([C@H](C)C(=O)NCC(F)(F)C(F)(F)F)cc1F)C(C1CC1)C1CC1]

N-((S)-1,1-dicyclopropyl-3-((2-fluoro-4-((S)-1-oxo-1-((2,2,3,3,3-pentafluoropropyl)amino)propan-2-yl)phenyl)amino)-3-oxopropan-2-yl)-1-isopropyl-1H-pyrazole-5-carboxamide (44). Prepared according to General Scheme C, employing methyl (S)-2-(4-amino-3-fluorophenyl)propanoate and (S)-2-((tert-butoxycarbonyl)amino)-3,3-dicyclopropylpropanoic acid followed by 1-isopropyl-1H-pyrazole-5-carboxylic acid and 2,2,3,3,3-pentafluoropropan-1-amine hydrochloride. LCMS [M+H]⁺=602.2 m % z.

### Example 46: Synthesis of Compound 45

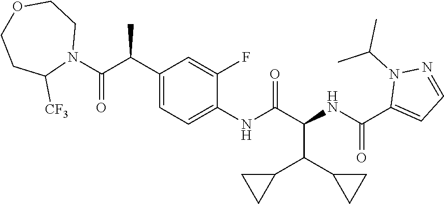 [SMILES: CC(C)n1nccc1C(=O)N[C@H](C(=O)Nc1ccc([C@H](C)C(=O)N2CCOCCC2C(F)(F)F)cc1F)C(C1CC1)C1CC1]

N-((2S)-1,1-dicyclopropyl-3-((2-fluoro-4-((2S)-1-oxo-1-(5-(trifluoromethyl)-1,4-oxazepan-4-yl)propan-2-yl)phenyl)amino)-3-oxopropan-2-yl)-1-isopropyl-1H-pyrazole-5-carboxamide (45). Prepared according to General Scheme C, employing methyl (S)-2-(4-amino-3-fluorophenyl)propanoate and (S)-2-((tert-butoxycarbonyl)amino)-3.3-dicyclopropylpropanoic acid followed by 1-isopropyl-1H-pyrazole-5-carboxylic acid and 5-(trifluoromethyl)-1,4-oxazepane hydrochloride. LCMS [M+H]⁺=622.2 m/z.

### Example 47: Synthesis of Compound 46

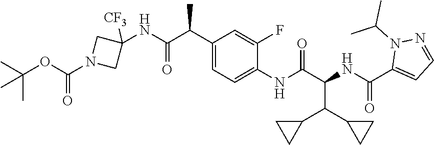 [SMILES: CC(C)n1nccc1C(=O)N[C@H](C(=O)Nc1ccc([C@H](C)C(=O)NC2(C(F)(F)F)CN(C(=O)OC(C)(C)C)C2)cc1F)C(C1CC1)C1CC1]

tert-butyl 3-((S)-2-(4-((S)-3,3-dicyclopropyl-2-(1-isopropyl-1H-pyrazole-5-carboxamido)propanamido)-3-fluorophenyl)propanamido)-3-(trifluoromethyl)azetidine-1-carboxylate (46). Prepared according to General Scheme C. employing methyl (S)-2-(4-amino-3-fluorophenyl)propanoate and (S)-2-((tert-butoxycarbonyl)amino)-3,3-dicyclopropylpropanoic acid followed by 1-isopropyl-1H-pyrazole-5-carboxylic acid and name. LCMS [M+H]⁺=693.2 m/z.

### Example 48: Synthesis of Compound 47

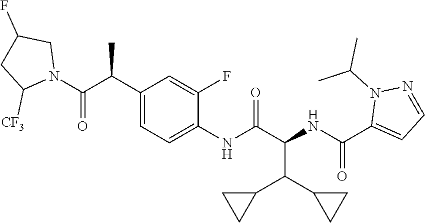 [SMILES: CC(C)n1nccc1C(=O)N[C@H](C(=O)Nc1ccc([C@H](C)C(=O)N2CC(F)CC2C(F)(F)F)cc1F)C(C1CC1)C1CC1]

N-((2S)-1,1-dicyclopropyl-3-((2-fluoro-4-((2S)-1-(4-fluoro-2-(trifluoromethyl)pyrrolidin-1-yl)-1-oxopropan-2-yl)phenyl)amino)-3-oxopropan-2-yl)-1-isopropyl-1H-pyrazole-5-carboxamide (47). Prepared according to General Scheme C. employing methyl (S)-2-(4-amino-3-fluorophenyl)propanoate and (S)-2-((tert-butoxycarbonyl)amino)-3,3-dicyclopropylpropanoic acid followed by 1-isopropyl-1H-pyrazole-5-carboxylic acid and 4-fluoro-2-(trifluoromethyl)pyrrolidine hydrochloride. LCMS [M+H]⁺=610.1 m/z.

### Example 49: Synthesis of Compound 48

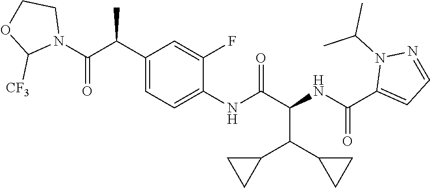 [SMILES: CC(C)n1nccc1C(=O)N[C@H](C(=O)Nc1ccc([C@H](C)C(=O)N2CCOC2C(F)(F)F)cc1F)C(C1CC1)C1CC1]

N-((2S)-1,1-dicyclopropyl-3-((2-fluoro-4-((2S)-1-oxo-1-(2-(trifluoromethyl)oxazolidin-3-yl)propan-2-yl)phenyl)amino)-3-oxopropan-2-yl)-1-isopropyl-1H-pyrazole-5-carboxamide (48). Prepared according to General Scheme C, employing methyl (S)-2-(4-amino-3-fluorophenyl)propanoate and (S)-2-((tert-butoxycarbonyl)amino)-3,3-dicyclopropylpropanoic acid followed by 1-isopropyl-1H-pyrazole-5-carboxylic acid and 2-(trifluoromethyl)oxazolidine. The title compound was isolated as the first eluting isomer by silica gel chromatography, eluting with 0-100% EtOAc in hexanes. LCMS [M+H]⁺=594.1 m/z.

### Example 50: Synthesis of Compound 49

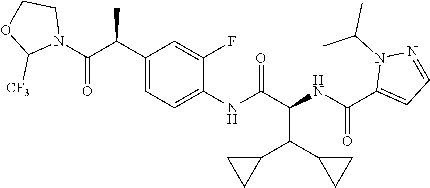 [SMILES: CC(C)n1nccc1C(=O)N[C@H](C(=O)Nc1ccc([C@H](C)C(=O)N2CCOC2C(F)(F)F)cc1F)C(C1CC1)C1CC1]

N-((2S)-1,1-dicyclopropyl-3-((2-fluoro-4-((2S)-1-oxo-1-(2-(trifluoromethyl)oxazolidin-3-yl)propan-2-yl)phenyl)amino)-3-oxopropan-2-yl)-1-isopropyl-1H-pyrazole-5-carboxamide (49). Prepared according to General Scheme C, employing methyl (S)-2-(4-amino-3-fluorophenyl)propanoate and (S)-2-((tert-butoxycarbonyl)amino)-3,3-dicyclopropylpropanoic acid followed by 1-isopropyl-1H-pyrazole-5-carboxylic acid and 2-(trifluoromethyl)oxazolidine. The title compound was isolated as the second eluting isomer by silica gel chromatography, eluting with 0-100% EtOAc in hexanes. LCMS [M+H]⁺=594.1 m/z.

### Example 51: Synthesis of Compound 50

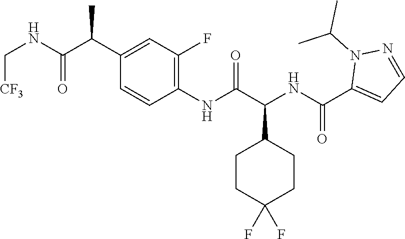 [SMILES: CC(C)n1nccc1C(=O)N[C@H](C(=O)Nc1ccc([C@H](C)C(=O)NCC(F)(F)F)cc1F)C1CCC(F)(F)CC1]

N-((S)-1-(4,4-difluorocyclohexyl)-2-((2-fluoro-4-((S)-1-oxo-1-((2,2,2-trifluoroethyl)amino)propan-2-yl)phenyl)amino)-2-oxoethyl)-1-isopropyl-1H-pyrazole-5-carboxamide (50). Prepared according to General Scheme C. employing methyl (S)-2-(4-amino-3-fluorophenyl)propanoate and ((S)-2-((tert-butoxycarbonyl)amino)-2-(4,4-difluorocyclohexyl)acetic acid followed by 1-isopropyl-1H-pyrazole-5-carboxylic acid and 2,2,2-trifluoroethan-1-amine hydrochloride. LCMS [M+H]⁺=590.1 m/z.

### Example 52: Synthesis of Compound 51

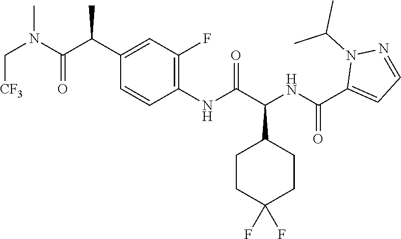 [SMILES: CC(C)n1nccc1C(=O)N[C@H](C(=O)Nc1ccc([C@H](C)C(=O)N(C)CC(F)(F)F)cc1F)C1CCC(F)(F)CC1]

N-((S)-1-(4,4-difluorocyclohexyl)-2-((2-fluoro-4-((S)-1-(methyl(2,2,2-trifluoroethyl)amino)-1-oxopropan-2-yl)phenyl)amino)-2-oxoethyl)-1-isopropyl-1H-pyrazole-5-carboxamide (51). Prepared according to General Scheme C, employing methyl (S)-2-(4-amino-3-fluorophenyl)propanoate and ((S)-2-((tert-butoxycarbonyl)amino)-2-(4,4-difluorocyclohexyl)acetic acid followed by 1-isopropyl-1H-pyrazole-5-carboxylic acid and 2,2,2-trifluoro-N-methylethan-1-amine hydrochloride. LCMS [M+H]⁺=576.1 m/z.

### Example 53: Synthesis of Compound 52

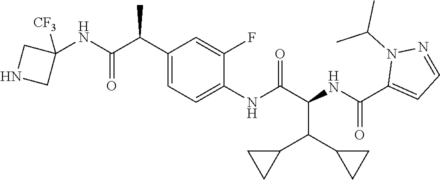 [SMILES: CC(C)n1nccc1C(=O)N[C@H](C(=O)Nc1ccc([C@H](C)C(=O)NC2(C(F)(F)F)CNC2)cc1F)C(C1CC1)C1CC1]

N-((S)-1.1-dicyclopropyl-3-((2-fluoro-4-((S)-1-oxo-1-((3-(trifluoromethyl)azetidin-3-yl)amino)propan-2-yl)phenyl)amino)-3-oxopropan-2-yl)-1-isopropyl-1H-pyrazole-5-carboxamide (52). Prepared from tert-butyl 3-((S)-2-(4-((S)-3,3-dicyclopropyl-2-(1-isopropyl-1H-pyrazole-5-carboxamido)propanamido)-3-fluorophenyl)propanamido)-3-(trifluoromethyl)azetidine-1-carboxylate in Compound 52 as exemplified in General Procedure C. LCMS [M+H]⁺=593.1 m/z.

### Example 54: Synthesis of Compound 53

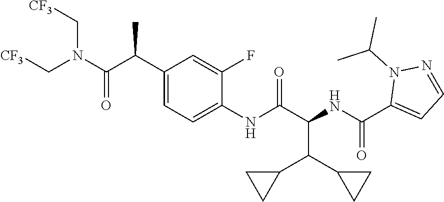 [SMILES: CC(C)n1nccc1C(=O)N[C@H](C(=O)Nc1ccc([C@H](C)C(=O)N(CC(F)(F)F)CC(F)(F)F)cc1F)C(C1CC1)C1CC1]

N-((S)-1-((44(S)-1-(bis(2,2,2-trifluoroethyl)amino)-1-oxopropan-2-yl)-2-fluorophenyl)amino)-3,3-dicyclopropyl-1-oxopropan-2-yl)-1-isopropyl-1H-pyrazole-5-carboxamide (53). Prepared according to General Scheme C, employing methyl (S)-2-(4-amino-3-fluorophenyl)propanoate and (S)-2-((tert-butoxycarbonyl)amino)-3,3-dicyclopropylpropanoic acid followed by 1-isopropyl-1H-pyrazole-5-carboxylic acid and bis(2,2,2-trifluoroethyl)amine hydrochloride. LCMS [M+H]⁺=620.1 m/z.

### Example 55: Synthesis of Compound 54

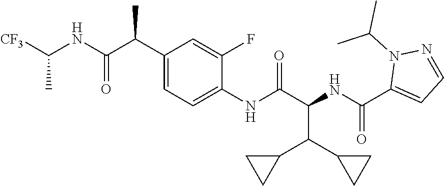 [SMILES: CC(C)n1nccc1C(=O)N[C@H](C(=O)Nc1ccc([C@H](C)C(=O)N[C@H](C)C(F)(F)F)cc1F)C(C1CC1)C1CC1]

N-((S)-1,1-dicyclopropyl-3-((2-fluoro-4-((S)-1-oxo-1-(((R)-1,1,1-trifluoropropan-2-yl)amino)propan-2-yl)phenyl)amino)-3-oxopropan-2-yl)-1-isopropyl-1H-pyrazole-5-carboxamide (54). Prepared according to General Scheme C, employing methyl (S)-2-(4-amino-3-fluorophenyl)propanoate and (S)-2-((tert-butoxycarbonyl)amino)-3,3-dicyclopropylpropanoic acid followed by 1-isopropyl-1H-pyrazole-5-carboxylic acid and (R)-1,1,1-trifluoropropan-2-amine hydrochloride. LCMS [M+H]⁺=566.1 m/z.

### Example 56: Synthesis of Compound 55

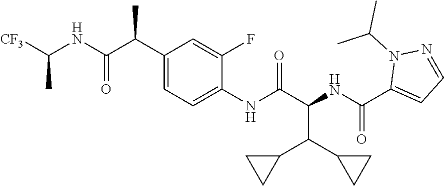 [SMILES: CC(C)n1nccc1C(=O)N[C@H](C(=O)Nc1ccc([C@H](C)C(=O)N[C@@H](C)C(F)(F)F)cc1F)C(C1CC1)C1CC1]

N-((S)-1,1-dicyclopropyl-3-((2-fluoro-4-((S)-1-oxo-1-(((S)-1,1,1-trifluoropropan-2-yl)amino)propan-2-yl)phenyl)amino)-3-oxopropan-2-yl)-1-isopropyl-1H-pyrazole-5-carboxamide (55). Prepared according to General Scheme C. employing methyl (S)-2-(4-amino-3-fluorophenyl)propanoate and (S)-2-((tert-butoxycarbonyl)amino)-3,3-dicyclopropylpropanoic acid followed by 1-isopropyl-1H-pyrazole-5-carboxylic acid and (S)-1,1,1-trifluoropropan-2-amine hydrochloride. LCMS [M+H]⁺=566.1 m/z.

### Example 57: Synthesis of Compound 56

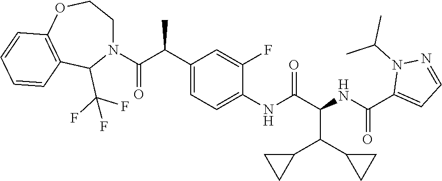 [SMILES: CC(C)n1nccc1C(=O)N[C@H](C(=O)Nc1ccc([C@H](C)C(=O)N2CCOc3ccccc3C2C(F)(F)F)cc1F)C(C1CC1)C1CC1]

N-((2S)-1,1-dicyclopropyl-3-((2-fluoro-4-((2S)-1-oxo-1-(5-(trifluoromethyl)-2,3-dihydrobenzo[f][1,4] oxazepin-4(5H)-yl)propan-2-yl)phenyl)amino)-3-oxopropan-2-yl)-1-isopropyl-1H-pyrazole-5-carboxamide (56). Prepared according to General Scheme C, employing methyl (S)-2-(4-amino-3-fluorophenyl)propanoate and (S)-2-((tert-butoxycarbonyl)amino)-3,3-dicyclopropylpropanoic acid followed by 1-isopropyl-1H-pyrazole-5-carboxylic acid and 5-(trifluoromethyl)-2,3,4,5-tetrahydrobenzo[f][1,4]oxazepine. LCMS [M+H]⁺=670.1 m % z.

### Example 58: Synthesis of Compound 57

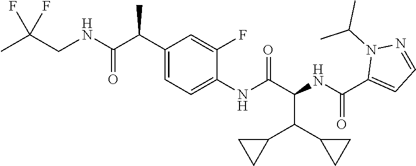 [SMILES: CC(C)n1nccc1C(=O)N[C@H](C(=O)Nc1ccc([C@H](C)C(=O)NCC(C)(F)F)cc1F)C(C1CC1)C1CC1]

N-((S)-1,1-dicyclopropyl-3-((4-((S)-1-((2,2-difluoropropyl)amino)-1-oxopropan-2-yl)-2-fluorophenyl)amino)-3-oxopropan-2-yl)-1-isopropyl-1H-pyrazole-5-carboxamide (57). Prepared according to General Scheme C. employing methyl (S)-2-(4-amino-3-fluorophenyl)propanoate and (S)-2-((tert-butoxycarbonyl)amino)-3,3-dicyclopropylpropanoic acid followed by 1-isopropyl-1H-pyrazole-5-carboxylic acid and 2,2-difluoropropan-1-amine hydrochloride. LCMS [M+H]⁺=548.2 Example 59: Synthesis of Compound 58

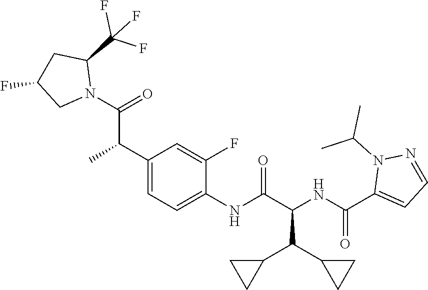 [SMILES: CC(C)n1nccc1C(=O)N[C@H](C(=O)Nc1ccc([C@H](C)C(=O)N2C[C@H](F)C[C@H]2C(F)(F)F)cc1F)C(C1CC1)C1CC1]

N-((S)-1,1-dicyclopropyl-3-((2-fluoro-4-((S)-1-((2S,4R)-4-fluoro-2-(trifluoromethyl)pyrrolidin-1-yl)-1-oxopropan-2-yl)phenyl)amino)-3-oxopropan-2-yl)-1-isopropyl-1H-pyrazole-5-carboxamide (58). Prepared according to General Scheme C, employing methyl (S)-2-(4-amino-3-fluorophenyl)propanoate and (S)-2-((tert-butoxycarbonyl)amino)-3,3-dicyclopropylpropanoic acid followed by 1-isopropyl-1H-pyrazole-5-carboxylic acid and (2S,4R)-4-fluoro-2-(trifluoromethyl)pyrrolidine hydrochloride. LCMS [M+H]⁺=610.1 i m.

### Example 60: Synthesis of Compound 59

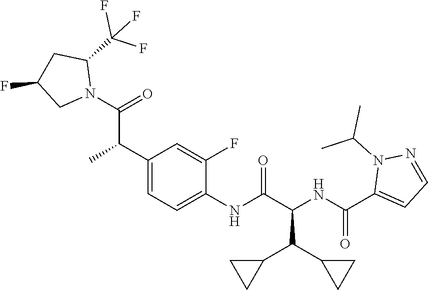 [SMILES: CC(C)n1nccc1C(=O)N[C@H](C(=O)Nc1ccc([C@H](C)C(=O)N2C[C@@H](F)C[C@@H]2C(F)(F)F)cc1F)C(C1CC1)C1CC1]

N-((S)-1,1-dicyclopropyl-3-((2-fluoro-4-((S)-1-((2R⁴S)-4-fluoro-2-(trifluoromethyl)pyrrolidin-1-yl)-1-oxopropan-2-yl)phenyl)amino)-3-oxopropan-2-yl)-1-isopropyl-1H-pyrazole-5-carboxamide (59). Prepared according to General Scheme C, employing methyl (S)-2-(4-amino-3-fluorophenyl)propanoate and (S)-2-((tert-butoxycarbonyl)amino)-3,3-dicyclopropylpropanoic acid followed by 1-isopropyl-1H-pyrazole-5-carboxylic acid and (2R,4S)-4-fluoro-2-(trifluoromethyl)pyrrolidine hydrochloride. LCMS [M+H]⁺=610. 1i m/z.

### Example 61: Synthesis of Compound 60

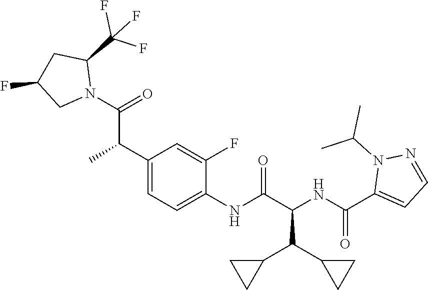 [SMILES: CC(C)n1nccc1C(=O)N[C@H](C(=O)Nc1ccc([C@H](C)C(=O)N2C[C@@H](F)C[C@H]2C(F)(F)F)cc1F)C(C1CC1)C1CC1]

N-((S)-1,1-dicyclopropyl-3-((2-fluoro-4-((S)-1-((2S,4S)-4-fluoro-2-(trifluoromethyl)pyrrolidin-1-yl)-1-oxopropan-2-yl)phenyl)amino)-3-oxopropan-2-yl)-1-isopropyl-1H-pyrazole-5-carboxamide (60). Prepared according to General Scheme C, employing methyl (S)-2-(4-amino-3-fluorophenyl)propanoate and (S)-2-((tert-butoxycarbonyl)amino)-3,3-dicyclopropylpropanoic acid followed by 1-isopropyl-1H-pyrazole-5-carboxylic acid and (2S,4S)-4-fluoro-2-(trifluoromethyl)pyrrolidine hydrochloride in an exemplified procedure with T3P.

LCMS [M+H]⁺=610.1 m/z.

### Example 62: Synthesis of Compound 61

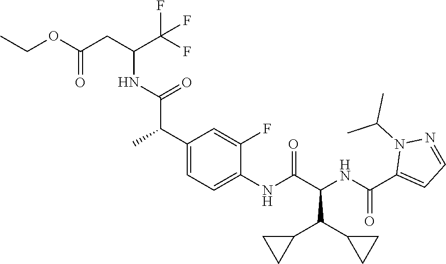 [SMILES: CCOC(=O)CC(NC(=O)[C@@H](C)c1ccc(NC(=O)[C@@H](NC(=O)c2ccnn2C(C)C)C(C2CC2)C2CC2)c(F)c1)C(F)(F)F]

ethyl 3-((S)-2-(4-((S)-3,3-dicyclopropyl-2-(1-isopropyl-1H-pyrazole-5-carboxamido)propanamido)-3-fluorophenyl)propanamido)-4,4,4-trifluorobutanoate (61). Prepared according to General Scheme C, employing methyl (S)-2-(4-amino-3-fluorophenyl)propanoate and (S)-2-((tert-butoxycarbonyl)amino)-3,3-dicyclopropylpropanoic acid followed by 1-isopropyl-1H-pyrazole-5-carboxylic acid and ethyl 3-amino-4,4,4-trifluoro-butanoate. LCMS [M+H]⁺=638.2 m/z.

### Example 63: Synthesis of Compound 62

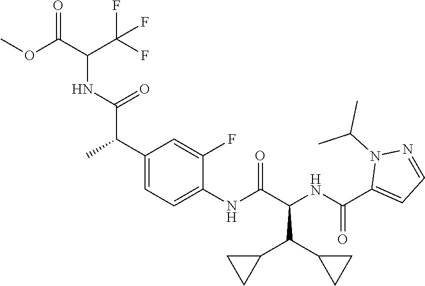 [SMILES: COC(=O)C(NC(=O)[C@@H](C)c1ccc(NC(=O)[C@@H](NC(=O)c2ccnn2C(C)C)C(C2CC2)C2CC2)c(F)c1)C(F)(F)F]

Methyl 2-jl(2S)-2-j4-[[(2S)-3,3-dicyclopropyl-2-[(2-isopropylpyrazole-3-carbonyl)amino]propanoyl]amino]-3-fluoro-phenyl]propanoyl]amino]-3,3,3-trifluoro-propanoate (62). Prepared according to General Scheme C, employing methyl (S)-2-(4-amino-3-fluorophenyl)propanoate and (S)-2-((tert-butoxycarbonyl)amino)-3,3-dicyclopropylpropanoic acid followed by 1-isopropyl-1H-pyrazole-5-carboxylic acid and methyl 2-amino-3,3,3-trifluoro-propanoate hydrochloride. LCMS [M+H]⁺=610.1 m/z.

### Example 64: Synthesis of Compound 63

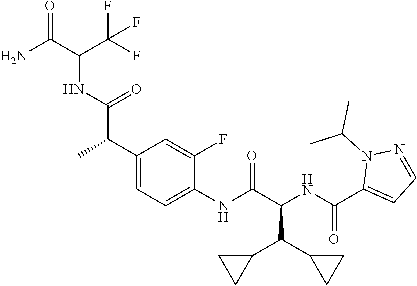 [SMILES: CC(C)n1nccc1C(=O)N[C@H](C(=O)Nc1ccc([C@H](C)C(=O)NC(C(N)=O)C(F)(F)F)cc1F)C(C1CC1)C1CC1]

N-((2S)-1-((4-((2S)-1-((3-amino-1,1-trifluoro-3-oxopropan-2-yl)amino)-1-oxopropan-2-yl)-2-fluorophenyl)amino)-3,3-dicyclopropyl-1-oxopropan-2-yl)-1-isopropyl-1H-pyrazole-5-carboxamide (63). Prepared according to General Scheme C, employing methyl (S)-2-(4-amino-3-fluorophenyl)propanoate and (S)-2-((tert-butoxycarbonyl)amino)-3,3-dicyclopropylpropanoic acid followed by 1-isopropyl-1H-pyrazole-5-carboxylic acid and 2-amino-3,3,3-trifluoro-propananude hydrochloride. LCMS [M+H]⁺=595.1 m/z.

### Example 65: Synthesis of Compound 64

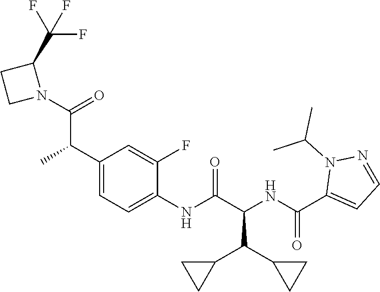 [SMILES: CC(C)n1nccc1C(=O)N[C@H](C(=O)Nc1ccc([C@H](C)C(=O)N2CC[C@H]2C(F)(F)F)cc1F)C(C1CC1)C1CC1]

N-((S)-1,1-dicyclopropyl-3-((2-fluoro-4-((S)-1-oxo-1-((S)-2-(trifluoromethyl)azetidin-1-yl)propan-2-yl)phenyl)amino)-3-oxopropan-2-yl)-1-isopropyl-1H-pyrazole-5-carboxamide (64). Prepared according to General Scheme C, employing methyl (S)-2-(4-amino-3-fluorophenyl)propanoate and (S)-2-((tert-butoxycarbonyl)amino)-3,3-dicyclopropylpropanoic acid followed by 1-isopropyl-1H-pyrazole-5-carboxylic acid and (S)-2-(trifluoromethyl)azetidine tosylate. LCMS [M+H]⁺=578.1 m/z.

### Example 66: Synthesis of Compound 65

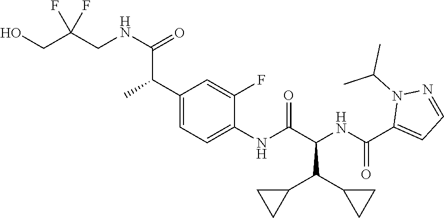 [SMILES: CC(C)n1nccc1C(=O)N[C@H](C(=O)Nc1ccc([C@H](C)C(=O)NCC(F)(F)CO)cc1F)C(C1CC1)C1CC1]

N-((S)-1,1-dicyclopropyl-3-((4-((S)-1-((2,2-difluoro-3-hydroxypropyl)amino)-1-oxopropan-2-yl)-2-fluorophenyl)amino)-3-oxopropan-2-yl)-1-isopropyl-1H-pyrazole-5-carboxamide (65). Prepared according to General Scheme C, employing methyl (S)-2-(4-amino-3-fluorophenyl)propanoate and (S)-2-((tert-butoxycarbonyl)amino)-3,3-dicyclopropylpropanoic acid followed by 1-isopropyl-1H-pyrazole-5-carboxylic acid and 3-amino-2,2-difluoro-propan-1-ol.

LCMS [M+H]⁺=564.2 m/z.

### Example 67: Synthesis of Compound 66

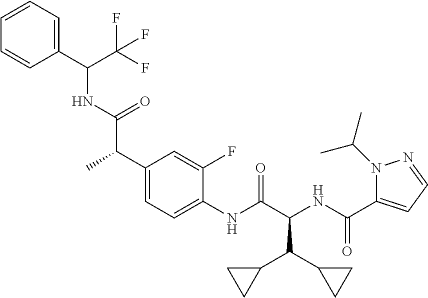 [SMILES: CC(C)n1nccc1C(=O)N[C@H](C(=O)Nc1ccc([C@H](C)C(=O)NC(c2ccccc2)C(F)(F)F)cc1F)C(C1CC1)C1CC1]

N-((2S)-1,1-dicyclopropyl-3-((2-fluoro-4-((2S)-1-oxo-1-((2,2,2-trifluoro-1-phenylethyl)amino)propan-2-yl)phenyl)amino)-3-oxopropan-2-yl)-1-isopropyl-1H-pyrazole-5-carboxamide (66). Prepared according to General Scheme C. employing methyl (S)-2-(4-amino-3-fluorophenyl)propanoate and (S)-2-((tert-butoxycarbonyl)amino)-3,3-dicyclopropylpropanoic acid followed by 1-isopropyl-1H-pyrazole-5-carboxylic acid and 2,2,2-trifluoro-1-phenyl-ethanamine hydrochloride. LCMS [M+H]⁺=628.1 m/z.

### Example 68: Synthesis of Compound 67

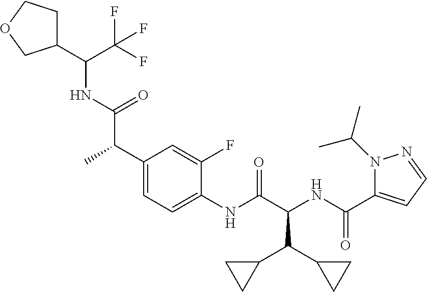 [SMILES: CC(C)n1nccc1C(=O)N[C@H](C(=O)Nc1ccc([C@H](C)C(=O)NC(C2CCOC2)C(F)(F)F)cc1F)C(C1CC1)C1CC1]

N-[(1S)-1-(dicyclopropylmethyl)-2-[2-fluoro-4-[(1S)-1-methyl-2-oxo-2-[(2,2,2-trifluoro-1-tetrahydrofuran-3-yl-ethyl)amino]ethyl]anilino]-2-oxo-ethyl]-2-isopropyl-pyrazole-3-carboxamide (67). Prepared according to General Scheme C. employing methyl (S)-2-(4-amino-3-fluorophenyl)propanoate and (S)-2-((tert-butoxycarbonyl)amino)-3,3-dicyclopropylpropanoic acid followed by 1-isopropyl-1H-pyrazole-5-carboxylic acid and 2,2,2-trifluoro-1-tetrahydrofuran-3-yl-ethanamine. LCMS [M+H]⁺=622.2 m/z.

### Example 69: General Scheme A-Synthesis of Compounds 68-101

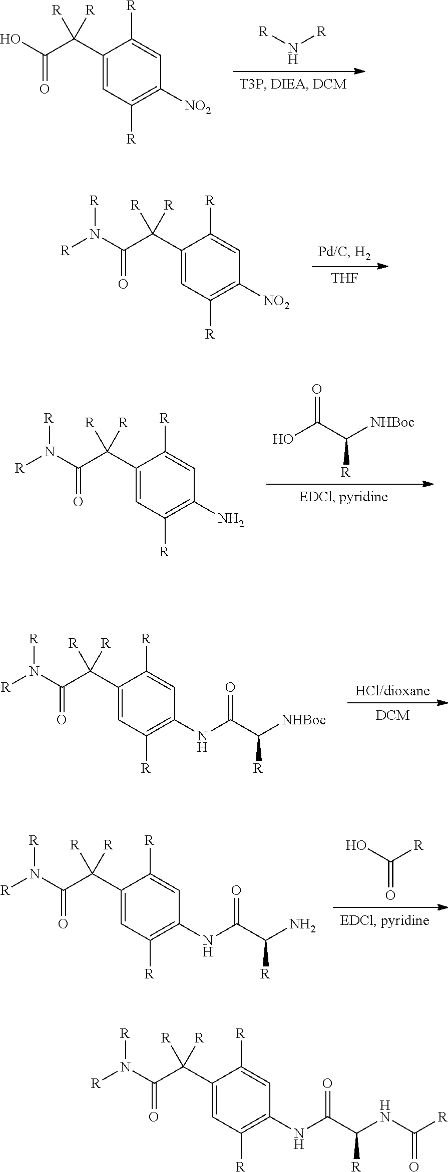

Examples synthesized according to General Scheme A employ a carboxylic acid and an amine in the first step followed by an amino acid in step 3 and a carboxylic acid in step 5. Starting materials are listed in order of use.

### Example 70: Synthesis of Compound 68

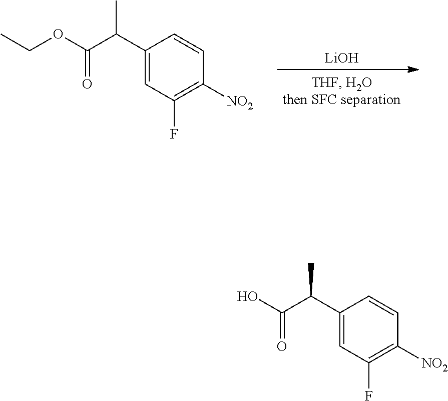 [SMILES: CCOC(=O)C(C)c1ccc([N+](=O)[O-])c(F)c1 ; "LiOH" ; "THF, H2Othen SFC separation" ; C[C@H](C(=O)O)c1ccc([N+](=O)[O-])c(F)c1]

(S)-2-(3-fluoro-4-nitrophenyl)propanoic acid. To a solution of ethyl 2-(3-fluoro-4-nitrophenyl)propanoate (5.00 g, 20.7 mmol, 1.00 eq) in THF (50.0 mL) and H₂O (20.0 mL) was added LiOH (1.99 g, 82.2 mmol, 4.00 eq). The reaction mixture was stirred at 10° C. for 3 h before concentrating under reduced pressure. The residue was diluted with H₂O (50.0 mL) and adjusted pH=4 with HCl (1.00 M), then extracted with ethyl acetate. The combined organic layers were dried over Na₂SO₄, filtered, and concentrated under reduced pressure to give a residue, which was purified by prep-SFC (column: DAICEL CHIRALPAK AD-H (250 mm×30 mm, 5 um), mobile phase: 15% 11:1 heptane:EtOH]) to afford (S)-2-(3-fluoro-4-nitrophenyl)propanoic acid (2.50 g, 11.7 mmol, 56.6% yield) as the second eluting isomer and yellow oil.

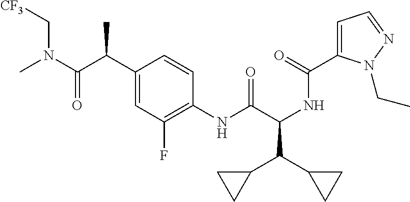 [SMILES: CCn1nccc1C(=O)N[C@H](C(=O)Nc1ccc([C@H](C)C(=O)N(C)CC(F)(F)F)cc1F)C(C1CC1)C1CC1]

N-((S)-1,1-dicyclopropyl-3-((2-fluoro-4-((S)-1-(methyl(2,2,2-trifluoroethyl)amino)-1-oxopropan-2-yl)phenyl)amino)-3-oxopropan-2-yl)-1-ethyl-1H-pyrazole-5-carboxamide (68). Prepared according to General Scheme A, employing (S)-2-(3-fluoro-4-nitrophenyl)propanoic acid and 2,2,2-trifluoro-N-methylethan-1-amine hydrochloride followed by (S)-2-((tert-butoxycarbonyl)amino)-3,3-dicyclopropylpropanoic acid and 1-ethyl-1H-pyrazole-5-carboxylic acid. LCMS [M+H]⁺=552.2 m % z.

### Example 71: Synthesis of Compound 69

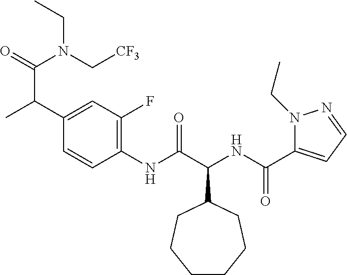 [SMILES: CCN(CC(F)(F)F)C(=O)C(C)c1ccc(NC(=O)[C@@H](NC(=O)c2ccnn2CC)C2CCCCCC2)c(F)c1]

N-((1S)-1-cycloheptyl-2-((4-(1-(ethyl(2,2,2-trifluoroethyl)amino)-1-oxopropan-2-yl)-2-fluorophenyl)amino)-2-oxoethyl)-1-ethyl-1H-pyrazole-5-carboxamide (69). Prepared according to General Scheme A, employing 2-(3-fluoro-4-nitrophenyl)propanoic acid and N-ethyl-2,2,2-trifluoroethan-1-amine hydrochloride followed by (S)-2-((tert-butoxycarbonyl)amino)-2-cycloheptylacetic acid and 1-ethyl-1H-pyrazole-5-carboxylic acid. The title compound was isolated as the first eluting, single stereoisomer by chiral SFC purification (column: DAICEL CHIRALPAK AD (250 mm×30 mm, 10 um): mobile phase: 20% [0.1% NH₃H₂O in IPA]). LCMS [M+H]⁺=568.2 m. z.

### Example 72: Synthesis of Compound 70

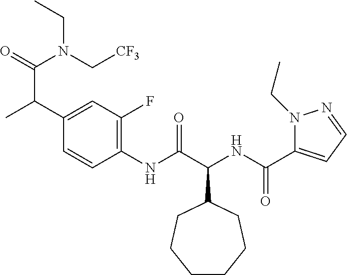 [SMILES: CCN(CC(F)(F)F)C(=O)C(C)c1ccc(NC(=O)[C@@H](NC(=O)c2ccnn2CC)C2CCCCCC2)c(F)c1]

N-((1S)-1-cycloheptyl-2-((4-(1-(ethyl(2,2,2-trifluoroethyl)amino)-1-oxopropan-2-yl)-2-fluorophenyl)amino)-2-oxoethyl)-1-ethyl-1H-pyrazole-5-carboxamide (70). Prepared according to General Scheme A. employing 2-(3-fluoro-4-nitrophenyl)propanoic acid and N-ethyl-2,2,2-trifluoroethan-1-amine hydrochloride followed by (S)-2-((tert-butoxycarbonyl)amino)-2-cycloheptylacetic acid and 1-ethyl-1H-pyrazole-5-carboxylic acid. The title compound was isolated as the second eluting, single stereoisomer by chiral SFC purification (column: DAICEL CHIRALPAK AD (250 mm×30 mm, 10 um); mobile phase: 20% [0.1% NH₃H₂O in IPA]). LCMS [M+H]⁺=568.2 m/z.

### Example 73: Synthesis of Compound 71

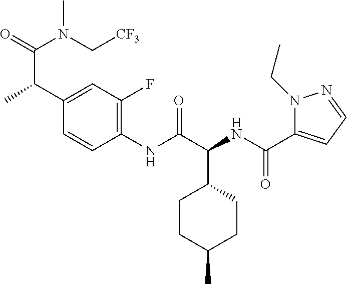 [SMILES: CCn1nccc1C(=O)N[C@H](C(=O)Nc1ccc([C@H](C)C(=O)N(C)CC(F)(F)F)cc1F)[C@H]1CC[C@H](C)CC1]

-ethyl-N-((S)-2-((2-fluoro-4-((S)-1-(methyl(2,2,2-trifluoroethyl)amino)-1-oxopropan-2-yl)phenyl)amino)-1-((1r,4S)-4-methylcyclohexyl)-2-oxoethyl)-1H-pyrazole-5-carboxamide (71). Prepared according to General Scheme A, employing 2-(3-fluoro-4-nitrophenyl)propanoic acid and N-methyl-2,2,2-trifluoroethan-1-amine hydrochloride followed by (S)-2-((tert-butoxycarbonyl)amino)-2-((1r,4S)-4-methylcyclohexyl)acetic acid and 1-ethyl-1H-pyrazole-5-carboxylic acid. LCMS [M+H]⁺=554.3 m/z.

### Example 74: Synthesis of Compound 72

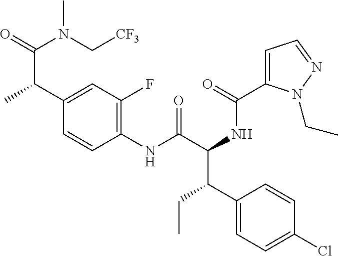 [SMILES: CC[C@@H](c1ccc(Cl)cc1)[C@H](NC(=O)c1ccnn1CC)C(=O)Nc1ccc([C@H](C)C(=O)N(C)CC(F)(F)F)cc1F]

N-((2S,3S)-3-(4-chlorophenyl)-1-((2-fluoro-4-((S)-1-(methyl(2,2,2-trifluoroethyl)amino)-1-oxopropan-2-yl)phenyl)amino)-1-oxopentan-2-yl)-1-ethyl-1H-pyrazole-5-carboxamide (72). Prepared according to General Scheme A. employing 2-(3-fluoro-4-nitrophenyl)propanoic acid and N-methyl-2,2,2-trifluoroethan-1-amine hydrochloride followed by (2S,3S)-2-((tert-butoxycarbonyl)amino)-3-(4-chlorophenyl)pentanoic acid and 1-ethyl-1H-pyrazole-5-carboxylic acid. LCMS [M+H]⁺=610.3 m/z.

### Example 75: Synthesis of Compound 73

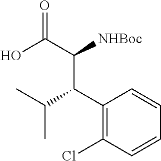 [SMILES: CC(C)[C@@H](c1ccccc1Cl)[C@H](NC(=O)OC(C)(C)C)C(=O)O]

(2S,3S)-2-((tert-butoxycarbonyl)amino)-3-(2-chlorophenyl)-4-methylpentanoic acid. Prepared according to WO 2019/223718. Absolute configuration was elucidated by single crystal x-ray diffraction of a related diastereomer as the tight ion pair with (S)-phenylethylamine, (2S,3R)-2-((tert-butoxycarbonyl)amino)-3-(2-chlorophenyl)-4-methylpentanoic acid, crystals grown by slow evaporation of 15 mg title compound with 5.4 mg (S)-phenylethylamine in 0.9 mL of 1:2 ACN/water at 45° C., [parameters: (CuKα), space group=12, a=12.6111(4), b=6.5141(3), c=30.7194(10), and alpha=90°, beta=90.390(3°), gamma=90° ].

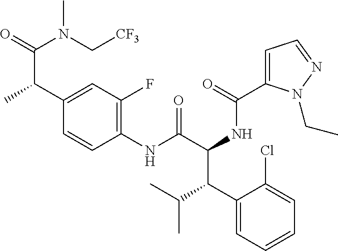 [SMILES: CCn1nccc1C(=O)N[C@H](C(=O)Nc1ccc([C@H](C)C(=O)N(C)CC(F)(F)F)cc1F)[C@H](c1ccccc1Cl)C(C)C]

N-((2S,3S)-3-(2-chlorophenyl)-1-((2-fluoro-4-((S)-1-(methyl(2,2,2-trifluoroethyl)amino)-1-oxopropan-2-yl)phenyl)amino)-4-methyl-1-oxopentan-2-yl)-1-ethyl-1H-pyrazole-5-carboxamide (73). Prepared according to General Scheme A, employing 2-(3-fluoro-4-nitrophenyl)propanoic acid and N-methyl-2,2,2-trifluoroethan-1-amine hydrochloride followed by (2S,3S)-2-((tert-butoxycarbonyl)amino)-3-(2-chlorophenyl)-4-methylpentanoic acid and 1-ethyl-1H-pyrazole-5-carboxylic acid. LCMS [M+H]⁺=624.2 m/z.

### Example 76: Synthesis of Compound Example 74

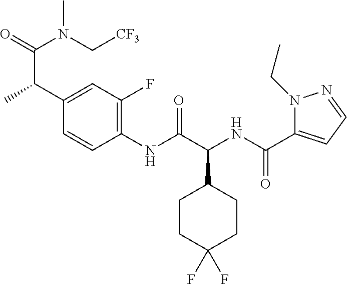 [SMILES: CCn1nccc1C(=O)N[C@H](C(=O)Nc1ccc([C@H](C)C(=O)N(C)CC(F)(F)F)cc1F)C1CCC(F)(F)CC1]

N-((S)-1-(4,4-difluorocyclohexyl)-2-((2-fluoro-4-((S)-1-(methyl(2,2,2-trifluoroethyl)amino)-1-oxopropan-2-yl)phenyl)amino)-2-oxoethyl)-1-ethyl-1H-pyrazole-5-carboxamide (74). Prepared according to General Scheme A, employing 2-(3-fluoro-4-nitrophenyl)propanoic acid and N-methyl-2,2,2-trifluoroethan-1-amine hydrochloride followed by (S)-2-((tert-butoxycarbonyl)amino)-2-(4,4-difluorocyclohexyl)acetic acid and 1-ethyl-1H-pyrazole-5-carboxylic acid. LCMS [M+H]⁺=576.3 m/z.

### Example 77: Synthesis of Compound 75

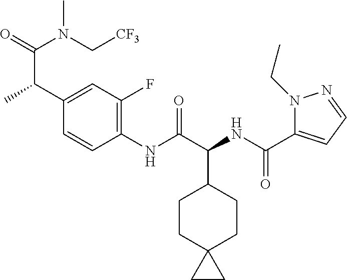 [SMILES: CCn1nccc1C(=O)N[C@H](C(=O)Nc1ccc([C@H](C)C(=O)N(C)CC(F)(F)F)cc1F)C1CCC2(CC1)CC2]

1-ethyl-N-((S)-2-((2-fluoro-4-((S)-1-(methyl(2,2,2-trifluoroethyl)amino)-1-oxopropan-2-yl)phenyl)amino)-2-oxo-1-(spiro[2.5]octan-6-yl)ethyl)-1H-pyrazole-5-carboxamide (75). Prepared according to General Scheme A, employing 2-(3-fluoro-4-nitrophenyl)propanoic acid and N-methyl-2,2,2-trifluoroethan-1-amine hydrochloride followed by (S)-2-(((benzyloxy)carbonyl)amino)-2-(spiro[2.5]octan-6-yl)acetic acid and 1-ethyl-1H-pyrazole-5-carboxylic acid. LCMS [M+H]⁺=566.4 m/z.

### Example 78: Synthesis of Compound 76

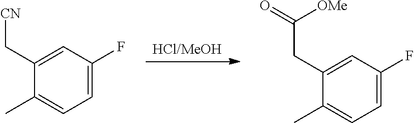

Synthesis of methyl 2-(5-fluoro-2-methylphenyl)acetate. A mixture of 2-(5-fluoro-2-methylphenyl)acetonitrile (4.00 g, 26.8 mmol, 1.00 eq) in HCl/MeOH (4.00 M, 70.0 mL, 10.4 eq) was stirred at 80° C. for 16 h under N₂ atmosphere. The reaction mixture was concentrated and diluted with H₂O. The aqueous layer was adjusted with sat. aq. NaHCO₃ to pH=8 and then extracted with EtOAc. The combined organic layers were dried over anhydrous Na₂SO₄, filtered, and concentrated under reduced pressure to afford methyl 2-(5-fluoro-2-methylphenyl)acetate (4.3 g, 24 mmol, 88% yield) as yellow oil. ¹H NMR (400 MHz, CDCl₃) δ 7.15-7.11 (m, 1H), 6.95-6.88 (m, 2H), 3.71 (s, 3H), 3.62 (s, 2H), 2.27 (s, 3H).

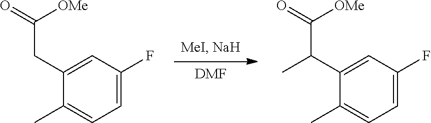

methyl 2-(5-fluoro-2-methylphenyl)propanoate. To a solution of methyl 2-(5-fluoro-2-methylphenyl)acetate (3.90 g, 21.4 mmol, 1.00 eq) in DMF (50.0 mL) was added NaH (980 mg, 24.5 mmol, 60.0% purity, 1.14 eq) at 0° C. the mixture was stirred at 0° C. for 0.2)₂, then methyl iodide (2.73 g, 19.2 mmol, 1.20 mL, 0.900 eq) was added. The mixture was stirred at 10° C. for 1.5 h. The reaction mixture was quenched with 1.00 M HCl at 0° C. and extracted with EtOAc. The combined organic layers were dried over Na₂SO₄, filtered, and concentrated under reduced pressure to give a residue, which was purified by prep-HPLC (0-100% ACN in water with 0.05% HCl) to afford a residue. The residue was concentrated and extracted with EtOAc, then the organic layers were dried over anhydrous Na₂SO₄. filtered, and concentrated under reduced pressure to afford methyl 2-(5-fluoro-2-methylphenyl)propanoate (2.80 g, 14.2 mmol, 66.6% yield) was obtained as yellow oil. ¹H NMR. (400 MHz, CDCl₃) δ 7.14-7.10 (m, 1H), 6.99 (dd, J₁=2.8 Hz, J₂=10.4 Hz, 1H), 6.88-6.83 (m, 1H), 3.96-3.90 (m, 1H), 3.68 (s, 3H), 2.32 (s, 3H), 1.47 (d, J=8.0 Hz, 3H).

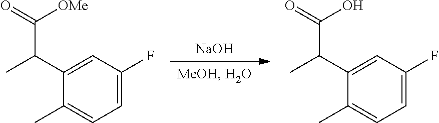

2-(5-fluoro-2-methylphenyl)propanoic acid. To a solution of methyl 2-(5-fluoro-2-methylphenyl)propanoate (2.80 g, 14.2 mmol, 1.00 eq) in MeOH (35.0 mL) was added a solution of NaOH (1.14 g, 28.5 mmol, 2.00 eq) in H₂O (8.00 mL) at 0° C. The mixture was stirred at 30° C. for 3 h. The reaction mixture was concentrated and diluted with H₂O. The aqueous layer was adjusted with 1.00 M HCl to pH=3, and then extracted with EtOAc. The combined organic layers were dried over anhydrous Na₂SO₄, filtered, and concentrated under reduced pressure to afford 2-(5-fluoro-2-methylphenyl)propanoic acid (2.45 g, 13.4 mmol, 94.2% yield) as yellow solid. ¹H NMR (400 MHz, CDCl₃) δ 11.4 (s, 1H), 7.15-7.11 (m, 1H), 7.03 (dd, J₁=2.8 Hz, J₂=12.8 Hz, 1H), 6.90-6.85 (m, 1H), 3.99-3.93 (m, 1H), 2.34 (s, 3H), 1.50 (d, J=7.2 Hz, 3H).

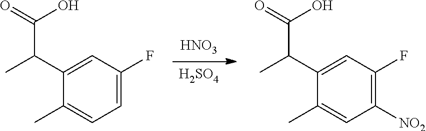

2-(5-fluoro-2-methyl-4-nitrophenyl)propanoic acid. To a solution of 2-(5-fluoro-2-methylphenyl)propanoic acid (2.45 g, 13.4 mmol, 1.00 eq) in H₂SO₄ (20.0 mL) was added HNO3 (1.82 g, 20.2 mmol, 1.30 mL, 70.0% purity, 1.50 eq) at 0° C. slowly, the mixture was stirred at 0° C. for 2 h. The mixture quenched with ice water and extracted with EtOAc, the combined organic layers were washed with brine, dried over Na₂SO₄, filtered, and concentrated to afford crude 2-(5-fluoro-2-methyl-4-nitrophenyl)propanoic acid (3.60 g) as yellow oil. ¹H NMR (400 MHz, CDCl₃) δ 7.91 (d, J=7.6 Hz, 1H), 7.31 (d. J=2.8 Hz, 1H), 4.16-4.12 (m, 1H), 2.43 (s, 3H), 1.57-1.56 (m, 3H).

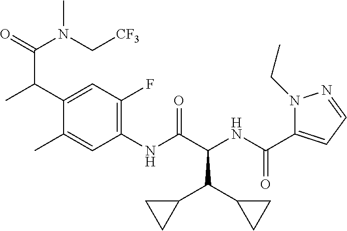 [SMILES: CCn1nccc1C(=O)N[C@H](C(=O)Nc1cc(C)c(C(C)C(=O)N(C)CC(F)(F)F)cc1F)C(C1CC1)C1CC1]

N-((2S)-1,1-dicyclopropyl-3-((2-fluoro-5-methyl-4-(1-(methyl(2,2,2-trifluoroethyl)amino)-1-oxopropan-2-yl)phenyl)amino)-3-oxopropan-2-yl)-1-ethyl-1H-pyrazole-5-carboxamide (76). Prepared according to General Scheme A, employing 2-(5-fluoro-2-methyl-4-nitrophenyl)propanoic acid and 2,2,2-trifluoro-N-methylethan-1-amine hydrochloride followed by (S)-2-((tert-butoxycarbonyl)amino)-3,3-dicyclopropylpropanoic acid and 1-ethyl-1H-pyrazole-5-carboxamide. The title compound was isolated as the first eluting, single stereoisomer by chiral SFC purification (column: DAICEL CHIRALPAK IC (250 mm×30 mm, 5 um); mobile phase: 30% 10.1% NNH₃H₂O in methanol1). LCMS [M+H]⁺=566.3 m/z Example 79: Synthesis of Compound 77

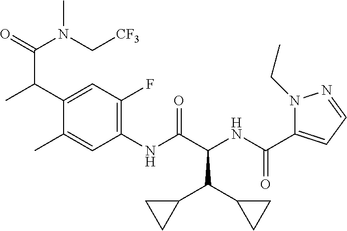 [SMILES: CCn1nccc1C(=O)N[C@H](C(=O)Nc1cc(C)c(C(C)C(=O)N(C)CC(F)(F)F)cc1F)C(C1CC1)C1CC1]

N-((2S)-1,1-dicyclopropyl-3-((2-fluoro-5-methyl-4-(1-(methyl(2,2,2-trifluoroethyl)amino)-1-oxopropan-2-yl)phenyl)amino)-3-oxopropan-2-yl)-1-ethyl-1 H-pyrazole-5-carboxamide (77). Prepared according to General Scheme A. employing 2-(5-fluoro-2-methyl-4-nitrophenyl)propanoic acid and 2,2,2-trifluoro-N-methylethan-1-amine hydrochloride followed by (S)-2-((tert-butoxycarbonyl)amino)-3,3-dicyclopropylpropanoic acid and 1-ethyl-1H-pyrazole-5-carboxamide. The title compound was isolated as the second eluting, single stereoisomer by chiral SFC purification (column: DAICEL CHIRALPAK IC (250 mm×30 mm, 5 um); mobile phase: 30% [0.1% NH₃H₂O in methanol]). LCMS [M+H]⁺=566.3 m/z.

### Example 80: Synthesis of Compound 78

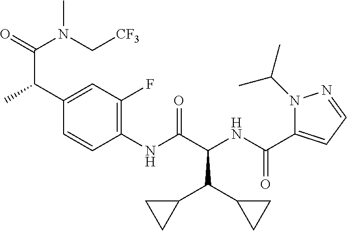 [SMILES: CC(C)n1nccc1C(=O)N[C@H](C(=O)Nc1ccc([C@H](C)C(=O)N(C)CC(F)(F)F)cc1F)C(C1CC1)C1CC1]

N-((S)-1,1-dicyclopropyl-3-((2-fluoro-4-((S)-1-(methyl(2,2,2-trifluoroethyl)amino)-1-oxopropan-2-yl)phenyl)amino)-3-oxopropan-2-yl)-1-isopropyl-1H-pyrazole-5-carboxamide (78). Prepared according to General Scheme A, employing (S)-2-(3-fluoro-4-nitrophenyl)propanoic acid and 2,2,2-trifluoro-N-methylethan-1-amine hydrochloride followed by (S)-2-((tert-butoxycarbonyl)amino)-3,3-dicyclopropylpropanoic acid and 1-isopropyl-1H-pyrazole-5-carboxylic acid. LCMS [M+HI]=566.2 m.

### Example 81: Synthesis of Compound 79

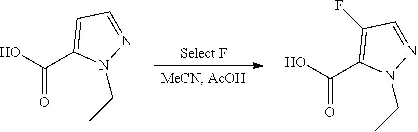

1-ethyl-4-fluoro-1H-pyrazole-5-carboxylic acid, 1-Ethyl-1H-pyrazole-5-carboxylic acid (0.500 g, 3.57 mmol, 1.00 eq) and Select F (2.53 g, 7.14 mmol, 2.00 eq) were added to a microwave reaction via] and diluted with MeCN (8.00 mL) and AcOH (3 mL). The sealed tube was heated at 110° C. for 6 h under microwave. The reaction mixture was diluted with H₂O and extracted with EtOAc. The combined organic layers were dried over Na₂SO₄, filtered, and concentrated under reduced pressure to give a residue, which was purified by prep-HPLC, eluting with 7-37% ACN in water with 0.225% formic acid to afford 1-ethyl-4-fluoro-1H-pyrazole-5-carboxylic acid (160 mg, 1.01 mmol, 28.3% yield) as yellow solid. ¹H NMR (400 MHz, CDCl₃) d 7.43 (d, J=4.4 Hz, 1H), 4.57-4.52 (m, 2H), 1.44 (t, J=7.2 Hz, 3H).

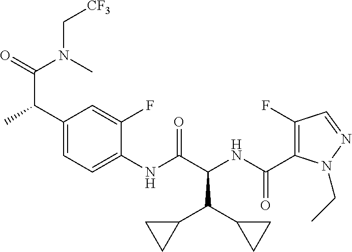 [SMILES: CCn1ncc(F)c1C(=O)N[C@H](C(=O)Nc1ccc([C@H](C)C(=O)N(C)CC(F)(F)F)cc1F)C(C1CC1)C1CC1]

N-((S)-1,1-dicyclopropyl-3-((2-fluoro-4-((S)-1-(methyl(2,2,2-trifluoroethyl)amino)-1-oxopropan-2-yl)phenyl)amino)-3-oxopropan-2-yl)-1-ethyl-4-fluoro-1H-pyrazole-5-carboxamide (79). Prepared according to General Scheme A, employing (S)-2-(3-fluoro-4-nitrophenyl)propanoic acid and 2,2,2-trifluoro-N-methylethan-1-amine hydrochloride followed by (S)-2-((tert-butoxycarbonyl)amino)-3,3-dicyclopropylpropanoic acid and 1-ethyl-4-fluoro-1H-pyrazole-5-carboxylic acid. LCMS [M+H]⁺=570.3 m/z.

### Example 82: Synthesis of Compound 80

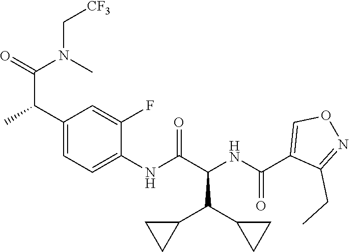 [SMILES: CCc1nocc1C(=O)N[C@H](C(=O)Nc1ccc([C@H](C)C(=O)N(C)CC(F)(F)F)cc1F)C(C1CC1)C1CC1]

N-((S)-1,1-dicyclopropyl-3-((2-fluoro-4-((S)-1-(methyl(2,2,2-trifluoroethyl)amino)-1-oxopropan-2-yl)phenyl)amino)-3-oxopropan-2-yl)-3-ethylisoxazole-4-carboxamide (80). Prepared according to General Scheme A, employing (S)-2-(3-fluoro-4-nitrophenyl)propanoic acid and 2,2,2-trifluoro-N-methylethan-1-amine hydrochloride followed by (S)-2-((tert-butoxycarbonyl)amino)-3,3-dicyclopropylpropanoic acid and 3-ethylisoxazole-4-carboxylic acid. LCMS [M+H]⁺=553.2 m/z.

### Example 83: Synthesis of Compound 81

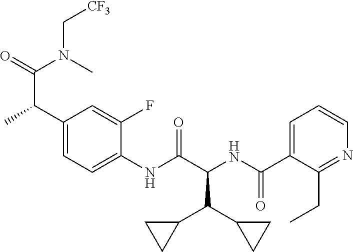 [SMILES: CCc1ncccc1C(=O)N[C@H](C(=O)Nc1ccc([C@H](C)C(=O)N(C)CC(F)(F)F)cc1F)C(C1CC1)C1CC1]

N-((S)-1,1-dicyclopropyl-3-((2-fluoro-4-((S)-1-(methyl(2,2,2-trifluoroethyl)amino)-1-oxopropan-2-yl)phenyl)amino)-3-oxopropan-2-yl)-2-ethylnicotinamide (81). Prepared according to General Scheme A, employing (S)-2-(3-fluoro-4-nitrophenyl)propanoic acid and 2,2,2-trifluoro-N-methylethan-1-amine hydrochloride followed by (S)-2-((tert-butoxycarbonyl)amino)-3,3-dicyclopropylpropanoic acid and 2-ethylnicotinic acid. LCMS [M+H]⁺=563.2 m/z.

### Example 84: Synthesis of Compound 82

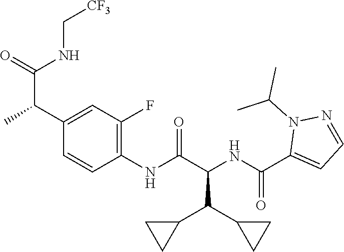 [SMILES: CC(C)n1nccc1C(=O)N[C@H](C(=O)Nc1ccc([C@H](C)C(=O)NCC(F)(F)F)cc1F)C(C1CC1)C1CC1]

N-((S)-1,1-dicyclopropyl-3-((2-fluoro-4-((S)-1-oxo-1-((2,2,2-trifluoroethyl)amino)propan-2-yl)phenyl)amino)-3-oxopropan-2-yl)-1-isopropyl-1H-pyrazole-5-carboxamide (82). Prepared according to General Scheme A, employing (S)-2-(3-fluoro-4-nitrophenyl)propanoic acid and 2,2,2-trifluoroethan-1-amine hydrochloride followed by (S)-2-((tert-butoxycarbonyl)amino)-3,3-dicyclopropylpropanoic acid and 1-isopropyl-1H-pyrazole-5-carboxylic acid. LCMS [M+H]⁺=552.2 m/z.

### Example 85: Synthesis of Compound 83

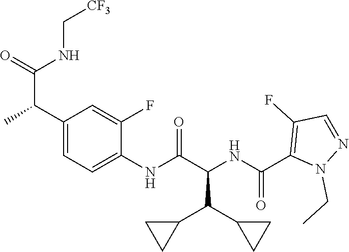 [SMILES: CCn1ncc(F)c1C(=O)N[C@H](C(=O)Nc1ccc([C@H](C)C(=O)NCC(F)(F)F)cc1F)C(C1CC1)C1CC1]

N-((S)-1,1-dicyclopropyl-3-((2-fluoro-4-((S)-1-oxo-1-((2,2,2-trifluoroethyl)amino)propan-2-yl)phenyl)amino)-3-oxopropan-2-yl)-1-ethyl-4-fluoro-1H-pyrazole-5-carboxamide (83). Prepared according to General Scheme A, employing (S)-2-(3-fluoro-4-nitrophenyl)propanoic acid and 2,2,2-trifluoroethan-1-amine hydrochloride followed by (S)-2-((tert-butoxycarbonyl)amino)-3,3-dicyclopropylpropanoic acid and 1-ethyl-4-fluoro-1H-pyrazole-5-carboxylic acid. LCMS [M+H]⁺=556.3.

### Example 86: Synthesis of Compound 84

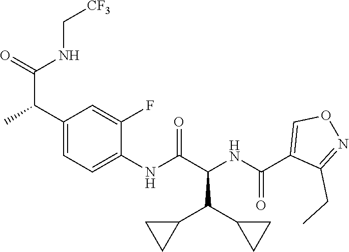 [SMILES: CCc1nocc1C(=O)N[C@H](C(=O)Nc1ccc([C@H](C)C(=O)NCC(F)(F)F)cc1F)C(C1CC1)C1CC1]

N-((S)-1,1-dicyclopropyl-3-((2-fluoro-4-((S)-1-oxo-1-((2,2,2-trifluoroethyl)amino)propan-2-yl)phenyl)amino)-3-oxopropan-2-yl)-3-ethylisoxazole-4-carboxamide (84). Prepared according to General Scheme A, employing (S)-2-(3-fluoro-4-nitrophenyl)propanoic acid and 2,2,2-trifluoroethan-1-amine hydrochloride followed by (S)-2-((tert-butoxycarbonyl)amino)-3,3-dicyclopropylpropanoic acid and 3-ethylisoxazole-4-carboxylic acid. LCMS [M+H]⁺=539.1.

### Example 87: Synthesis of Compound 85

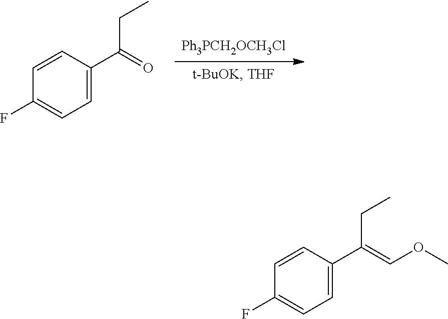

1-fluoro-4-(1-methoxybut-1-en-2-yl)benzene. To a solution of 1-(4-fluorophenyl)propan-1-one (400 g, 2.63 mol, 363 mL, 1.00 eq) in THF (2000 mL) was added t-BuOK (442 g, 3.94 mol, 1.50 eq) and (methoxymethyl)triphenylphosphonium chloride (1.17 kg, 3.42 mol, 1.30 eq). The mixture was stirred at 0-5° C. for 2 h. The reaction mixture was treated with water (2000 mL) and then diluted with EtOAc (2000 mL) and extracted with EtOAc (3×2000 mL). The combined organic layers were washed with brine (800 mL), dried over Na₂SO₄. filtered, and concentrated under reduced pressure to give a residue. The residue was purified by column chromatography (SiO₂, Petroleum ether/Ethyl acetate=100/1 to 50/1) to obtain 480 g as a light yellow residue, which was further purified by column chromatography (SiO₂, Petroleum ether: Ethyl acetate=10:1) to afford the title compound (320 g, 1.78 mol, 67.5% yield) as a light yellow oil. ¹H NMR (400 MHz, CDCl₃): δ 7.46-7.44 (m, 1H), 7.28-7.23 (m, 1H), 7.04-6.98 (m, 2H), 6.14 (d, J=48.0 Hz, 1H), 3.67 (d, J=23.2 Hz, 3H), 2.53-2.47 (m, 1H), 2.35-2.30 (m, 1H), 1.02-0.97 (m, 3H).

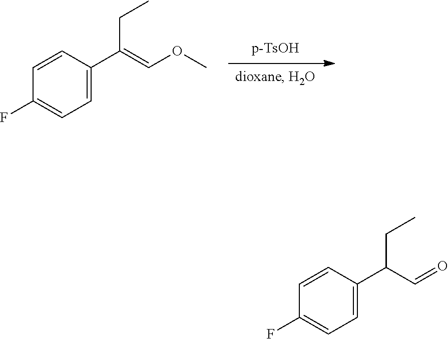

2-(4-fluorophenyl)butanal. To a solution of 1-fluoro-4-(1-methoxybut-1-en-2-yl)benzene (420 g, 2.33 mol, 1.00 eq) in dioxane (4000 mL) and H₂O (400 mL) was added TsOH (1.61 kg, 9.32 mol, 4.0x) eq). The mixture was stirred at 100-105° C. for 2 h. The reaction mixture was treated with water (4000 mL), and then diluted with EtOAc (4000 mL) and extracted with EtOAc (3×3000 mL). The combined organic layers were washed with sat. aq. NaHCO₃ (2000 mL) and brine (1000 mL), dried over Na₂SO₄, filtered, and concentrated under reduced pressure to afford the title compound (400 g, crude) as a light yellow oil. ¹H NMR (400 MHz, CDCb₃): δ 9.65 (s, 1H), 7.18-7.15 (m, 2H), 7.10-7.04 (m, 2H), 3.43-3.38 (m, 1H), 2.12-2.04 (m, 1H), 1.77-1.70 (m, 1H), 0.92-0.87 (m, 3H).

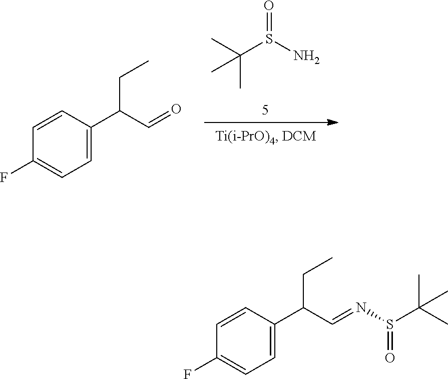

(S)-N-((E)-2-(4-fluorophenyl)butylidene)-2-methylpropane-2-sulfinamide. To a solution of 2-(4-fluorophenyl)butanal (400 g, 2.41 mol, 1.00 eq) in THF (3000 mL) and was added (S)-2-methylpropane-2-sulfinamide (350 g, 2.89 mol, 1.20 eq) and Ti(i-PrO)₄ (1.37 kg, 4.81 mol, 2.00 eq). The mixture was stirred at 70-80° C. for 2 h. The reaction mixture was treated with H₂O (5000 mL) and filtered, and the filtrate was extracted with EtOAc (3×5000 mL). The combined organic layers were washed with brine (3000 mL), dried over Na₂SO₄, filtered, and concentrated under reduced pressure to give a residue. The residue was purified by column chromatography (SiO₂, Petroleum ether/Ethyl acetate=50:1 to 10:1) to afford the title compound (450 g, 1.67 mol, 69.4% yield) as a light yellow oil. ¹H NMR (400 MHz, CDCl₃): δ 8.06-8.04 (m, 1H), 7.19-7.15 (m, 2H), 7.04-7.01 (m, 2H), 3.64-3.60 (m, 1H), 2.08-2.03 (m, 1H), 1.86-1.76 (m, 1H), 1.17 (d, J=24.0 Hz, 9H), 0.93-0.89 (m, 3H).

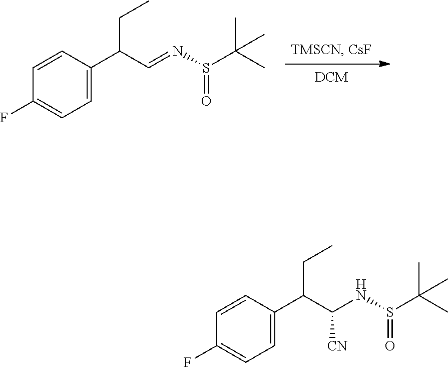

(S)-N-((1S)-1-cyano-2-(4-fluorophenyl)butyl)-2-methylpropane-2-sulfinamide. To a solution of (S)-N-((E)-2-(4-fluorophenyl)butylidene)-2-methylpropane-2-sulfinamide. To a solution of 2-(4-fluorophenyl)butanal (220 g, 817 mmol, 1.00 eq) in DCM (2000 mL) was added TMSCN (162 g, 1.63 mol, 204 mL, 2.00 eq), CsF (37.2 g, 245 mmol, 9.03 mL, 0.30 eq) and H₂O (2.94 g, 163 mmol, 2.94 mL, 0.20 eq). The mixture was stirred at 10-25° C. for 12 h. The reaction mixture was treated with water (2000 mL), and then diluted with DCM (2000 mL) and extracted with DCM (3×2000 mL). The combined organic layers were washed with brine (800 mL), dried over Na₂SO₄, filtered, and concentrated under reduced pressure to give a residue. The residue was purified by column chromatography (SiO₂, Petroleum ether:EtOAc=20:1 to 5:1) to afford the title compound (200 g, 674 mmol, 82.6% yield) as an off-white solid. ¹H NMR (400 MHz, CDCl₃): δ 7.25-7.22 (m, 1H), 7.18-7.13 (m, 1H), 7.03-7.00 (m, 2H), 4.35-4.19 (m, 1H), 3.63-3.46 (m, 1H), 2.92-2.81 (m, 1H), 1.98-1.73 (m, 2H), 1.11 (d, J=20.0 Hz, 9H), 0.85-0.77 (m, 3H).

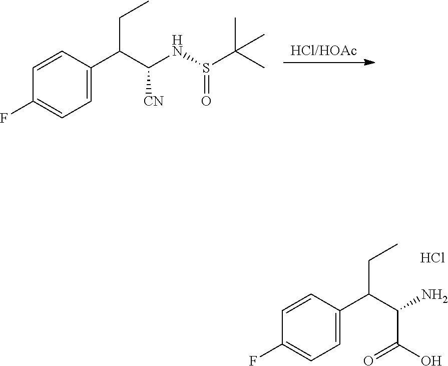 [SMILES: CCC(c1ccc(F)cc1)[C@@H](C#N)N[S@@](=O)C(C)(C)C ; "HCl/HOAc" ; CCC(c1ccc(F)cc1)[C@H](N)C(=O)O ; Cl]

(2S)-2-amino-3-(4-fluorophenyl)pentanoic acid hydrochloride salt. To a solution of (S)-N-((1S)-1-cyano-2-(4-fluorophenyl)butyl)-2-methylpropane-2-sulfinamide (200 g, 674 mmol, 1.00 eq) in AcOH (400 mL) was added HCl (8 M, 2000 mL, 23.7 eq). The mixture was stirred at 100-105° C. for 12 h. he reaction mixture concentrated under reduced pressure to give a residue (200 g, crude) as a brown oil. The residue was used for next step without further purification.

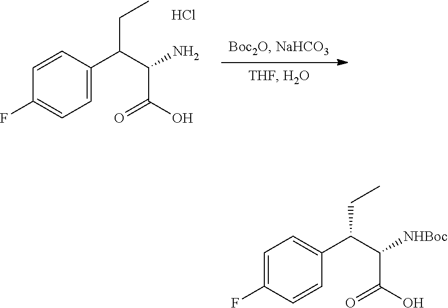

(2S,3S)-2-((tert-butoxycarbonyl)amino)-3-(4-fluorophenyl)pentanoic acid. To a solution of (2S)-2-amino-3-(4-fluorophenyl)pentanoic acid hydrochloride salt (200 g, crude) in THF (1200 mL) and H₂O (1200 mL) was added Boc₂O (413 g, 1.89 mol, 435 mL) and K₂CO₃ (523 g, 3.79 mol). The mixture was stirred at 15-25° C. for 3 h. The reaction mixture was adjust pH=3-4 with 1 M HCl, diluted with EtOAc (2000 mL), and extracted with EtOAc (3×2000 mL). The combined organic layers were washed with brine (800 mL), dried over Na₂SO₄, filtered, and concentrated under reduced pressure to give a residue. The residue was purified by column chromatography (SiO₂, Petroleum ether/Ethyl acetate=20/1 to 5/1), which was further purified by reversed-phase HPLC (column: Welch Ultimate XB—SiOH₂ 50×50, 10 um: mobile phase: 1Hexane-EtOH, 0.1% NH₃·H₂O]: B %: 10%-40%) and SFC (column: DAICELCHIRALPAK AD (250 mm×50 mm, 10 um); mobile phase: [0.1% NH₃ H₂O IPA]; B %: 25%) to afford a mixture of isomers (60.0 g, 186 mmol, 19.7% yield, 96.8% purity) as a light yellow oil. The title compound was isolated as a single stereoisomer by chiral SFC purification of 25.0 g of the mixture (column: DAICEL CHIRALPAK AD (250 mm×30 mam, 10 um): eluting with 15% of 0.1% N1HH₂O in ethanol). The title compound (12.0 g, 38.5 mmol, 48.0% yield) was obtained as the first eluting isomer and an off-white solid. LCMS: [M+Na|*=334.1; ¹H NMR (400 MHz, MeOD): δ 7.23-7.19 (m, 2H), 7.02-6.98 (m, 2H), 4.37-4.34 (m, 1H), 3.00-2.95 (m, 1H), 1.83-1.67 (m, 2H), 1.42 (s, 9H), 0.88-0.76 (m, 3H). Absolute configuration was elucidated by single crystal x-ray diffraction, crystals grown by slow evaporation of 17 mg title compound and 6.5 mg of (R)-phenylethylamine in 1 mL of 1:1 ACN/MeOH, Iparameters: (CuKα), space group ═P21, a=11.0606(3), b=5.51880(10), c=19.8399(5), and alpha=90°, beta=102.312(2°), gamma=90° ].

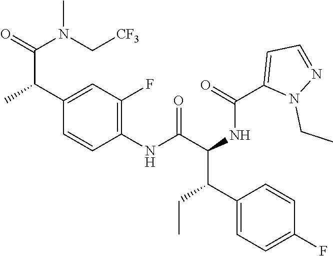 [SMILES: CC[C@@H](c1ccc(F)cc1)[C@H](NC(=O)c1ccnn1CC)C(=O)Nc1ccc([C@H](C)C(=O)N(C)CC(F)(F)F)cc1F]

-ethyl-N-((2S,3S)-1-((2-fluoro-4-((S)-1-(methyl(2,2,2-trifluoroethyl)amino)-1-oxopropan-2-yl)phenyl)amino)-3-(4-fluorophenyl)-1-oxopentan-2-yl)-1H-pyrazole-5-carboxamide (85). Prepared according to General Scheme A, employing (S)-2-(3-fluoro-4-nitrophenyl)propanoic acid and 2,2,2-trifluoro-N-methylethan-1-amine hydrochloride followed by (2S,3S)-2-((tert-butoxycarbonyl)amino)-3-(4-fluorophenyl)pentanoic acid and 1-ethyl-1H-pyrazole-5-carboxylic acid. LCMS [M+H]⁺=594.1 m/z.

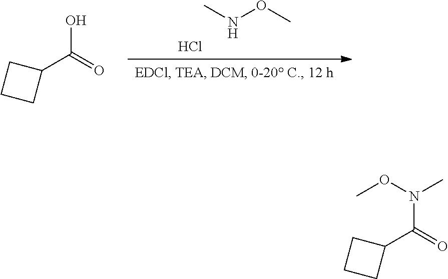

N-Methoxy-N-methylcyclobutanecarboxamide. To a solution of cyclobutanecarboxylic acid (75.0 g, 749 mmol, 71.4 mL, 1.00 eq) in DCM (350 mL) was added N,O-dimethylhydroxylanine hydrochloride (87.7 g, 899 mmol, 1.20 eq), EDCI (215 g, 1.12 mol, 1.50 eq), and TEA (114 g, 1.12 mol, 156 mL, 1.50 eq) at 0° C. The reaction mixture was stirred at RT for 12 h. The reaction mixture was treated with sat. NaHCO₃ and extracted with DCM. The combined organic layers were washed with 1 N HCl and brine, dried over Na₂SO₄. filtered, and concentrated under reduced pressure to afford N-methoxy-N-methylcyclobutanecarboxamide (98.0 g, 684 mmol, 91.3% yield) as a light yellow liquid. ¹H NMR (400 MHz, CDCl₃): δ: 3.64 (s, 3H), 3.46 (d, J=6.8 Hz, 1H), 3.16 (s, 3H), 2.35-2.29 (m, 2H), 2.14-2.10 (m, 2H), 1.96-1.81 (M, 2H).

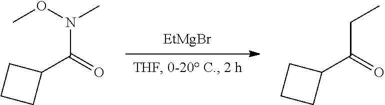

1-Cyclobutylpropan-1-one. To a solution of N-methoxy-N-methylcyclobutanecarboxamide (20.0 g, 140 mmol, 1.00 eq) in THF (100 mL) was added EtMgBr (3.00 M, 55.9 mL, 1.20 eq) at 0° C. The reaction mixture was stirred at RT for 2 h. The reaction was treated with sat. aqueous ammonium chloride and extracted with EtOAc. The combined organic layers were washed with water and brine, dried over Na₂SO₄, filtered, and concentrated under reduced pressure to afford 1-cyclobutylpropan-1-one (13.0 g, crude) as a light yellow liquid. ¹H NMR (400 MHz, CDCl₃): di: 3.29-3.22 (m, 1H), 2.40-2.33 (m, 2H), 2.22-2.11 (m, 4H), 2.04-1.94 (m, 2H), 1.04 (t, J=7.6 Hz, 3H).

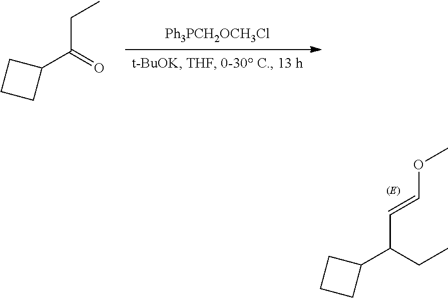

(E)-(1-Methoxypent-1-en-3-yl)cyclobutene. To a solution of (methoxymethyl)triphenylphosphonium chloride (49.5 g, 144 mmol, 1.20 eq) in THF (60.0 mL) was added dropwise t-BuOK (16.2 g, 144 mmol, 1.20 eq) in THF (60.0 mL) at 0° C. under N₂. After addition, the mixture was stirred at 5° C. for 1 h, and then 1-cyclobutylpropan-1-one (13.5 g, 120 mmol, 1.00 eq) was added dropwise. The resulting mixture was stirred at 30° C. for 12 h. The reaction was treated with H₂O and extracted with DCM. The combined organic layers were washed with brine, dried over Na₂SO₄, filtered a,nd concentrated under reduced pressure. The residue was purified by column chromatography (SiO₂, Petroleum ether: Ethyl acetate=50: 1) to afford (E)-(1-methoxypent-1-en-3-yl)cyclobutane (10.0 g, 71.3 mmol, 59.2% yield) as a light yellow liquid.

¹H NMR (400 MHz, CDCl₃): δ 5.67 (d, J=11.2 Hz, 1H), 3.54 (d, J=13.2 Hz, 3H), 2.91-2.86 (m, 1H), 2.05-2.01 (m, 4H), 2.00-1.85 (m, 2H), 1.84-1.83 (m, 2H), 1.00-0.93 (m, 3H).

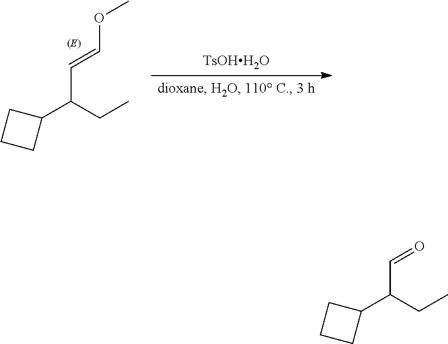

2-Cyclobutylbutanal. To a solution of (E)-(1-methoxypent-1-en-3-yl)cyclobutane (10.0 g, 71.3 mmol, 1.00 eq) in dioxane (50.0 mL) and H₂O (5.00 mL) was added TsOH₂O (54.3 g, 285 mmol, 4.00 eq). The mixture was stirred at 110° C. for 3 h. The reaction mixture was treated with sat. aq. NaHCO₃ and extracted with DCM. The combined organic layers were washed with brine, dried over Na₂SO₄, filtered, and concentrated under reduced pressure to afford 2-cyclobutylbutanal (10.0 g, crude) as a light yellow liquid. ¹H NMR (400 MHz, CDCl₃): δ: 9.53 (d, J=3.2 Hz, 1H), 2.49-2.47 (m, 1H), 2.19-2.06 (m, 1H), 1.91-1.82 (m, 2H), 1.82-1.79 (m, 4H), 1.55-1.51 (m, 2H), 0.88 d, J=7.2 Hz, 3H).

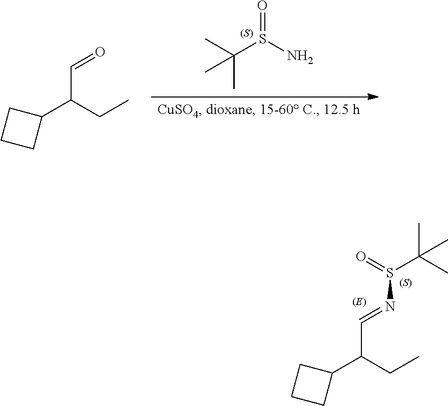

(S)-N-((E)-2-cyclobutylbutylidene)-2-methylpropane-2-sulfinamide. To a solution of 2-cyclobutylbutanal (10.0 g, 79.2 mmol, 1.00 eq) in dioxane (60.0 mL) was added (S)-2-methylpropane-2-sulfinamide (11.5 g, 95.1 mmol, 1.20 eq) at 15° C. under N₂. After addition, the mixture was stirred for 0.5)₂, and then CuSO4 (25.3 g, 158 mmol, 24.3 mL, 2.00 eq) was added dropwise. The resulting mixture was stirred at 60° C. for 12 h. The reaction mixture was filtered, and the filtered cake was washed with DCM. The organic phase was concentrated under reduced pressure. The residue was purified by column chromatography (SiO₂, Petroleum ether: Ethyl acetate=5: 1) to afford (S)-N-((E)-2-cyclobutylbutylidene)-2-methylpropane-2-sulfinamide (8.70 g, crude) as a light yellow oil.

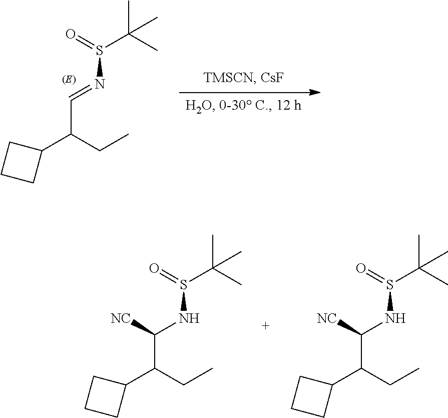

(S)-N-((1S)-1—Cyano-2-cyclobutylbutyl)-2-methylpropane-2-sulfinamide. To a solution of (S)-N-((E)-2-cyclobutylbutylidene)-2-methylpropane-2-sulfinamide (8.70 g, 37.9 mmol, 1.00 eq) in DCM (80.0 mL) was added CsF (1.15 g, 7.59 mmol, 280 μL, 0.20 eq) in H₂O (1.37 g, 75.9 mmol, 1.37 mL, 2.00 eq) and TMSCN (7.53 g, 75.9 mmol, 9.49 mL, 2.00 eq) at 0° C. The reaction mixture was stirred at 30° C. for 12 h. The reaction mixture was treated with H₂O and extracted with DCM. The combined organic layers were washed with brine, dried over Na₂SO₄, filtered, and concentrated under reduced pressure. The residue was purified by column chromatography (SiO₂.

Petroleum ether: Ethyl acetate=5:1 to 1:1). The residue was purified by chiral SFC purification (column: (250 mm×30 mm, 5 um): mobile phase: 5% to 40% [0.05% diethylamine in MeOH]) to afford the first eluting isomer of (S)-N-((1 S)-1-cyano-2-cyclobutylbutyl)-2-methylpropane-2-sulfinamide (3.20 g, 12.5 mmol, 32.8% yield, 99.7% purity) as a light yellow oil and the second eluting isomer of (S)-N-((1S)-1-cyano-2-cyclobutylbutyl)-2-methylpropane-2-sulfinamide (3.00 g, 11.7 mmol, 30.8% yield, 100% purity) as a light yellow oil. First eluting isomer: LCMS [M+H]⁺=257.0 m/z: ¹H NMR (400 MHz, CDCl₃): δ 4.18 (dd, J=3.6 Hz, 8.0 Hz, 1H), 3.55 (d, J=8.4 Hz, 1H), 2.39-2.37 (m, 1H), 2.12-2.08 (m, 2H), 1.90-1.62 (m, 5H), 1.50-1.47 (m, 2H), 1.25 (s, 9H), 0.97 (t, J=7.6 Hz, 3H). Second eluting isomer: LCMS [M+H]⁺=257.0 m/z; ¹H NMR (400 MHz, CDCl₃): δ 4.16 (dd, J=4.0 Hz, 9.6 Hz, 1H), 3.55 (d, J=9.6 Hz 1H), 2.46-2.41 (n, 1H), 2.09-2.07 (m, 2H), 1.84-1.75 (m, 5H), 1.41-1.39 (m, 2H), 1.24 (s, 9H), 0.97 (t, J=7.4 Hz, 3H).

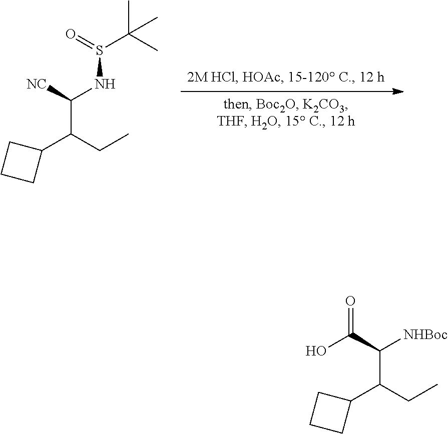 [SMILES: CCC(C1CCC1)[C@@H](C#N)N[S@@](=O)C(C)(C)C ; "2M HCl, HOAc, 15-120° C., 12 h" ; "then, Boc2O, K2CO3,THF, H2O, 15° C., 12 h" ; CCC(C1CCC1)[C@H](NC(=O)OC(C)(C)C)C(=O)O]

(2S)-2-((tert-Butoxycarbonyl)amino)-3-cyclobutylpentanoic acid. To a solution of the first eluting isomer of (S)-N-((1S)-1-cyano-2-cyclobutylbutyl)-2-methylpropane-2-sulfinamide (3.20 g, 12.5 mmol, 99.7% purity. 1.00 eq) in AcOH (6.70 g, 112 mmol, 6.38 mL, 8.97 eq) was added HCl (12.0 M, 37.2 mL, 35.9 eq) at RT. The mixture was stirred at 120° C. for 12 h. The reaction mixture was cooled to 15° C. and concentrated under reduced pressure to give the residue. The residue was treated with K₂C₀₃ (2.58 g, 18.7 mmol, 1.50 eq) in H₂O (100 mL) followed by Boc₂O (2.72 g, 12.5 mmol, 2.86 mL, 1.00 eq) in THF (50.0 mL) at RT and stirred for 12 h. The reaction mixture was extracted with DCM. The combined water layers were adjusted to pH=4-5 with sat. aq. citric acid solution, extracted with DCM, dried over Na₂SO₄, filtered, and concentrated under reduced pressure to give a residue. The residue was purified by prep-HPLC (eluting with ACN in water with 0.1% TFA) to afford (2S)-2-((tert-Butoxycarbonyl)amino)-3-cyclobutylpentanoic acid (668 mg, 2.44 mmol, 19.6% yield, 99.2% purity) as a single stereoisomer of unknown absolute configuration at the cyclobutyl center and white solid. LCMS [M+H-Boc]⁺=172.2 m/z; ¹H NMR (400 MHz, DMS₀-d6): δ 6.21 (d, J=6.0 Hz, 1H), 3.86-3.84 (m, 1H), 2.38-2.36 (m, 1H), 1.89-1.72 (m, 2H), 1.68-1.58 (m, 5H), 1.37 (s, 9H), 1.15-1.09 (m, 2H), 0.84 (t,J=7.4 Hz, 3H).

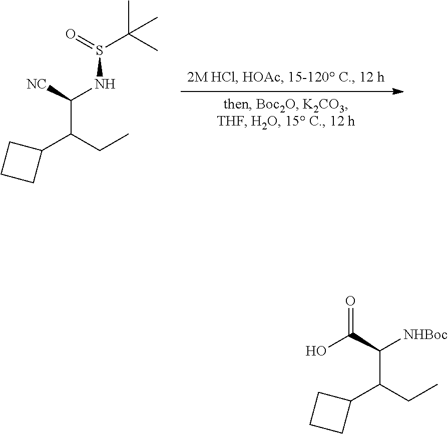 [SMILES: CCC(C1CCC1)[C@@H](C#N)N[S@@](=O)C(C)(C)C ; "2M HCl, HOAc, 15-120° C., 12 h" ; "then, Boc2O, K2CO3,THF, H2O, 15° C., 12 h" ; CCC(C1CCC1)[C@H](NC(=O)OC(C)(C)C)C(=O)O]

(2S)-2-((tert-Butoxycarbonyl)amino)-3-cyclobutylpentanoic acid. To a solution of the second eluting isomer of (S)-N-((1S)-1-cyano-2-cyclobutylbutyl)-2-methylpropane-2-sulfinamide (3.0( ) g, 11.7 mmol, 100% purity, 1.00 eq) in AcOH (6.30 g, 105 mmol, 6.00 mL, 8.97 eq) was added HCl (12.0 M, 35.0 mL, 35.9 eq) at RT. The mixture was stirred at 120° C. for 12 h. The reaction mixture was cooled to RT and concentrated under reduced pressure to give the residue. To a solution of residue and K₂CO₃ (2.43 g, 17.6 mmol, 1.50 eq) in H₂O (100 mL) was added Boc₂O (2.55 g, 11.7 mmol, 2.69 mL, 1.00 eq) in THF (50.0 mL) at RT. The mixture was stirred at RT for 12 h. The reaction mixture was extracted with DCM. The combined water layers were adjusted to pH=4-5 with sat. aq. citric acid solution, extracted with DCM, dried over Na₂SO₄, filtered, and concentrated under reduced pressure to give a residue. The residue was purified by prep-HPLC (eluting with ACN in water with 0.1% TFA) to afford (2S)-2-((tert-Butoxycarbonyl)amino)-3-cyclobutylpentanoic acid (1.26 g, 4.46 mmol, 38.1% yield, 96.1% purity) as a as a single stereoisomer of unknown absolute configuration at the cyclobutyl center and white solid. LCMS [M+H-Boc]⁺=172.2 m/z: ¹H NMR (400 MHz, DMS₀-d6): δ 6.02 (d, J=6.8 Hz, 1H), 3.74-3.72 (m, 1H), 2.22-2.08 (m, 1H), 1.95-1.92 (m, 2H), 1.71-1.62 (m, 5H), 1.23 (s, 9H), 1.22-1.15 (m, 2H), 0.79 (t, J=7.2 Hz, 3H).

### Example 88: Synthesis of Compound 86

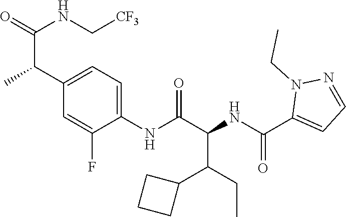 [SMILES: CCC(C1CCC1)[C@H](NC(=O)c1ccnn1CC)C(=O)Nc1ccc([C@H](C)C(=O)NCC(F)(F)F)cc1F]

N-((2S)-3-cyclobutyl-1-((2-fluoro-4-((S)-1-oxo-1-((2,2,2-trifluoroethyl)amino)propan-2-yl)phenyl)amino)-1-oxopentan-2-yl)-1-ethyl-1H-pyrazole-5-carboxamide (86). Prepared according to General Scheme A, employing (S)-2-(3-fluoro-4-nitrophenyl)propanoic acid and 2,2,2-trifluoroethan-1-amine hydrochloride followed by (2S)-2-((tert-butoxycarbonyl)amino)-3-cyclobutylpentanoic acid (first eluting isomer as (S)-N-((I S)-1-cyano-2-cyclobutylbutyl)-2-methylpropane-2-sulfinamide and single stereoisomer with unknown absolute stereochemistry at the cyclobutyl) and 1-ethyl-1H-pyrazole-5-carboxylic acid. The title compound is a single stereoisomer of unknown absolute stereochemistry at the cyclobutyl center. LCMS [M+H]⁺=540.1 m/z.

### Example 89: Synthesis of Compound 87

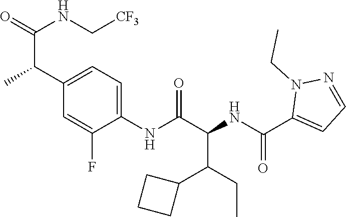 [SMILES: CCC(C1CCC1)[C@H](NC(=O)c1ccnn1CC)C(=O)Nc1ccc([C@H](C)C(=O)NCC(F)(F)F)cc1F]

N-((2S)-3-cyclobutyl-1-((2-fluoro-4-((S)-1-oxo-1-((2,2,2-trifluoroethyl)amino)propan-2-yl)phenyl)amino)-1-oxopentan-2-yl)-1-ethyl-1H-pyrazole-5-carboxamide (87). Prepared according to General Scheme A, employing (S)-2-(3-fluoro-4-nitrophenyl)propanoic acid and 2,2,2-trifluoroethan-1-amine hydrochloride followed by (2S)-2-((tert-butoxycarbonyl)amino)-3-cyclobutylpentanoic acid (first second isomer as (S)-N-((1S)-1-cyano-2-cyclobutylbutyl)-2-methylpropane-2-sulfinamide and single stereoisomer with unknown absolute stereochemistry at the cyclobutyl) and 1-ethyl-1H-pyrazole-5-carboxylic acid. The title compound is a single stereoisomer of unknown absolute stereochemistry at the cyclobutyl center. LCMS [M+H]⁺=540.1 m/z.

### Example 90: Synthesis of Compound 88

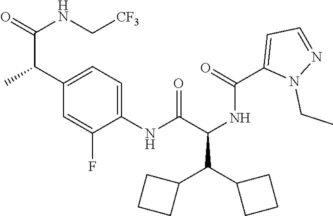 [SMILES: CCn1nccc1C(=O)N[C@H](C(=O)Nc1ccc([C@H](C)C(=O)NCC(F)(F)F)cc1F)C(C1CCC1)C1CCC1]

N-((S)-1,1-dicyclobutyl-3-((2-fluoro-4-((S)-1-oxo-1-((2,2,2-trifluoroethyl)amino)propan-2-yl)phenyl)amino)-3-oxopropan-2-yl)-1-ethyl-1H-pyrazole-5-carboxamide (88). Prepared according to General Scheme A, employing (S)-2-(3-fluoro-4-nitrophenyl)propanoic acid and 2,2,2-trifluoroethan-1-amine hydrochloride followed by (S)-2-((tert-butoxycarbonyl)amino)-3,3-dicyclobutylpropanoic acid (prepared according to WO 2020127685) and 1-ethyl-1H-pyrazole-5-carboxylic acid. LCMS [M+H]⁺=566.1 m/z.

### Example 91: Synthesis of Compound 89

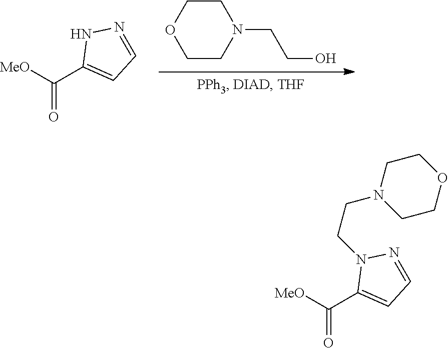

methyl 1-(2-morpholinoethyl)-1H-pyrazole-5-carboxylate. To a solution of methyl 1H-pyrazole-5-carboxylate (500 mg, 3.96 mmol, 1.00 eq), 2-morpholinoethan-1-ol (624 mg, 4.76 mmol, 583 uL, 1.20 eq) and PPh₃ (1.56 g, 5.95 mmol, 1.50 eq) in THF (5.00 mL) was added drop-wise DIAD (1.20 g, 5.95 mmol, 1.16 mL, 1.50 eq) at 0° C., then the mixture was stirred at RT for 4 h. The residue was diluted with H₂O and extracted with EtOAc. The combined organic layers were washed with brine, dried over Na₂SO₄, filtered, and concentrated under reduced pressure to give a residue. The residue was purified by column chromatography (SiO₂. Petroleum ether with 0-100% EtOAc) followed by prep-HPLC (12-42% ACN in water with 0.05% NH4OH) to afford methyl 1-(2-morpholinoethyl)-1H-pyrazole-5-carboxylate (0.600 g, 2.51 mmol, 63.3% yield) as a yellow oil. ¹H NMR (400 MHz, CDCl₃): δ 7.49 (s, 1H), 6.82 (t, J=2.0 Hz, 1H), 4.75 (t, J=6.4 Hz, 2H), 3.89 (d, J=2.0 Hz, 3H), 3.70 (s, 4H), 2.83 (t. .I=6.4 Hz, 2H), 2.57 (s, 4H).

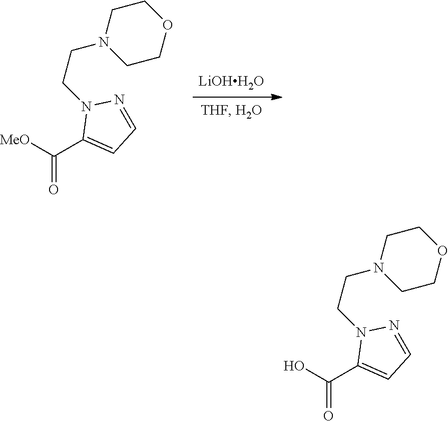

1-(2-morpholinoethyl)-1H-pyrazole-5-carboxylic acid. To a solution of methyl 1-(2-morpholinoethyl)-1H-pyrazole-5-carboxylate (0.600 g, 2.51 mmol, 1.0 Meq) in THF (10.0 mL) was added drop-wise a solution of LiOH—H₂O (158 mg, 3.76 mmol, 1.50 eq) in H₂O (5.00 mL) at 0° C., then the mixture was warmed to RT and allowed to stir for 2 h. The pH of the mixture was adjusted to 6 with 1M HCL The mixture was concentrated under reduced pressure to afford 1-(2-morpholinoethyl)-1H-pyrazole-5-carboxylic acid (0.500 g, 2.22 mmol, 88.5% yield) as a yellow oil.

LCMS [M+H]⁺=226.2 me.

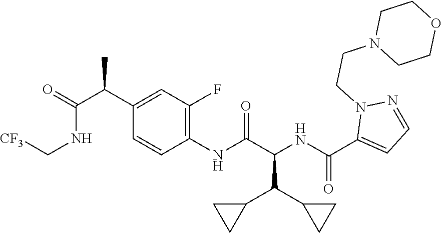 [SMILES: C[C@H](C(=O)NCC(F)(F)F)c1ccc(NC(=O)[C@@H](NC(=O)c2ccnn2CCN2CCOCC2)C(C2CC2)C2CC2)c(F)c1]

N-((S)-1,1-dicyclopropyl-3-((2-fluoro-4-((S)-1-oxo-1-((2,2,2-trifluoroethyl)amino)propan-2-yl)phenyl)amino)-3-oxopropan-2-yl)-1-(2-morpholinoethyl)-1H-pyrazole-5-carboxamide (89). Prepared according to General Scheme A, employing (S)-2-(3-fluoro-4-nitrophenyl)propanoic acid and 2,2,2-trifluoroethan-1-amine hydrochloride followed by (S)-2-((tert-butoxycarbonyl)amino)-3,3-dicyclopropylpropanoic acid and 1-(2-morpholinoethyl)-1H-pyrazole-5-carboxylic acid. LCMS [M+H]⁺=623.4m/z.

### Example 92 Synthesis of Compound 90

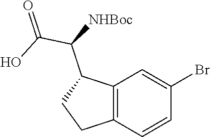 [SMILES: CC(C)(C)OC(=O)N[C@H](C(=O)O)[C@H]1CCc2ccc(Br)cc21]

(S)-2-((S)-6-bromo-2,3-dihydro-1H-inden-1-yl)-2-((tert-butoxycarbonyl)amino)acetic acid. Prepared according to literature procedures as reported in WO 2020127685. Absolute configuration was elucidated by comparison with single crystal x-ray diffraction of diastereomeric pair, below.

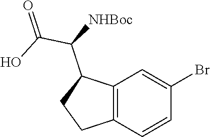 [SMILES: CC(C)(C)OC(=O)N[C@H](C(=O)O)[C@@H]1CCc2ccc(Br)cc21]

(S)-2-((R)-6-bromo-2,3-dihydro-1H-inden-1-yl)-2-((tert-butoxycarbonyl)amino)acetic acid. Prepared according to literature procedures as reported in WO 2020127685. Absolute configuration was elucidated by single crystal x-ray diffraction, crystals grown by slow evaporation of 15 mg title compound in 1 mL of 1:1 methanol/MTBE, [parameters: (CuKα), space group ═P2₁, a=9.62160(10), b=6.02750(10), c=14.06420(10), and alpha=90°, beta=97.9340(10°), gamma =90°].

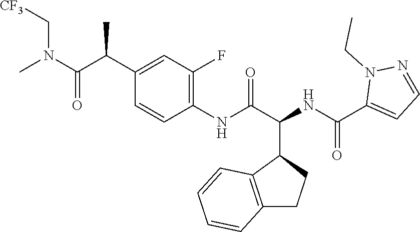 [SMILES: CCn1nccc1C(=O)N[C@H](C(=O)Nc1ccc([C@H](C)C(=O)N(C)CC(F)(F)F)cc1F)[C@H]1CCc2ccccc21]

N-((S)-1-((S)-2,3-dihydro-1H-inden-1-yl)-2-((2-fluoro-4-((S)-1-(methyl(2,2,2-trifluoroethyl)amino)-1-oxopropan-2-yl)phenyl)amino)-2-oxoethyl)-1-ethyl-1H-pyrazole-5-carboxamide (90). Prepared according to General Scheme A, employing 2-(5-fluoro-4-nitrophenyl)propanoic acid and 2,2,2-trifluoro-N-methylethan-1-amine hydrochloride followed by (S)-2-((S)-6-bromo-2,3-dihydro-1H-inden-1-yl)-2-((tert-butoxycarbonyl)amino)acetic acid and 1-ethyl-1H-pyrazole-5-carboxylic acid. The title compound was synthesized from with the following procedure: To a solution of N-((S)-1-((S)-6-bromo-2,3-dihydro-1H-inden-1-yl)-2-((2-fluoro-4-((S)-1-(methyl(2,2,2-trifluoroethyl)amino)-1-oxopropan-2-yl)phenyl)amino)-2-oxoethyl)-1-ethyl-1H-pyrazole-5-carboxamide (15.5 mg, 23.0 umol, 1.00 eq) in MeOH (0.0) mL) was added Pd/C (3 0 mg, 10% purity). The mixture was stirred at RT for 2 h under H₂ at 15 psi. The reaction mixture was diluted with MeOH, filtrated, and concentrated under reduced pressure to give a residue. The residue was purified by prep-TLC, eluting with 10:1 DCM: MeO to afford N-((S)-1-((S)-2,3-dihydro-1H-inden-1-yl)-2-((2-fluoro-4-((S)-1-(methyl(2,2,2-trifluoroethyl)amino)-1-oxopropan-2-yl)phenyl)amino)-2-oxoethyl)-1-ethyl-1H-pyrazole-5-carboxamide (7.62 mg, 12.5 umol, 54.4% yield) as a yellow solid. LCMS [M+H]⁺=574.3 m/z.

### Example 93: Synthesis of Compound 91

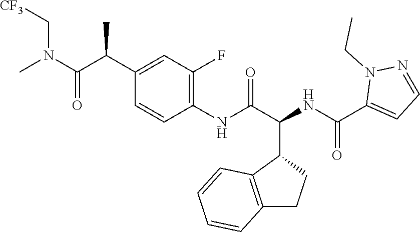 [SMILES: CCn1nccc1C(=O)N[C@H](C(=O)Nc1ccc([C@H](C)C(=O)N(C)CC(F)(F)F)cc1F)[C@@H]1CCc2ccccc21]

N-((S)-1-((R)-2,3-dihydro-1H-inden-1-yl)-2-((2-fluoro-4-((S)-1-(methyl(2,2,2-trifluoroethyl)amino)-1-oxopropan-2-yl)phenyl)amino)-2-oxoethyl)-1-ethyl-1H-pyrazole-5-carboxamide (91). N-((S)-1-((R)-6-bromo-2,3-dihydro-1H-inden-1-yl)-2-((2-fluoro-4-((S)-1-(methyl(2,2,2-trifluoroethyl)amino)-1-oxopropan-2-yl)phenyl)amino)-2-oxoethyl)-1-ethyl-1H-pyrazole-5-carboxamide was prepared according to General Scheme A, employing 2-(5-fluoro-4-nitrophenyl)propanoic acid and 2,2,2-trifluoro-N-methylethan-1-amine hydrochloride followed by (S)-2-((R)-6-bromo-2,3-dihydro-1H-inden-1-yl)-2-((tert-butoxycarbonyl)amino)acetic acid and 1-ethyl-1H-pyrazole-5-carboxylic acid. be title compound was synthesized from with the following procedure: To a solution of N-((S)-1-((R)-6-bromo-2,3-dihydro-1H-inden-1-yl)-2-((2-fluoro-4-((S)-1-(methyl(2,2,2-trifluoroethyl)amino)-1-oxopropan-2-yl)phenyl)amino)-2-oxoethyl)-1-ethyl-1H-pyrazole-5-carboxamide (8.00 mg, 11.9 umol, 1.00 eq) in MeOH (1.00 mL) was added Pd/C (10%, 0.010 g) under N₂ atmosphere. The mixture was stirred under H₂ (15 Psi) at 10° C. for 2 h. The reaction mixture was filtered and concentrated under reduced pressure to afford N-((S)-1-((R)-2,3-dihydro-1H-inden-1-yl)-2-((2-fluoro-4-((S)-1-(methyl(2,2,2-trifluoroethyl)amino)-1-oxopropan-2-yl)phenyl)amino)-2-oxoethyl)-1-ethyl-1H-pyrazole-5-carboxamide (6.39 mg, 10.7 umol, 89.56% yield) as a yellow solid. LCMS [M+H]⁺=574.4 m/z.

### Example 94: Synthesis of Compound 92

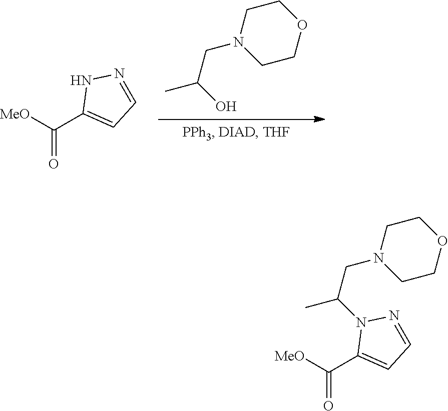

Methyl 1-(1-morpholinopropan-2-yl)-1H-pyrazole-5-carboxylate. To a solution of methyl 1H-pyrazole-5-carboxylate (500 mg, 3.44 mmol, 1.00 eq) and 1-morpholinopropan-2-ol (480 mg, 3.81 mmol, 1.11 eq) in THF (10.0 mL) was added D1AD (1.39 g, 6.89 mmol, 1.34 mL, 2.00 eq) and PPh.: (1.81 g, 6.89 mmol, 2.00 eq) at 0° C. The mixture was stirred at RT for 2 h. The reaction mixture was concentrated under reduced pressure, and the resulting residue was purified by column chromatography (SiO₂, Petroleum ether:EtOAc=10:1 to 8:1) followed by and prep-HPLC (eluting with 15-45% ACN in water with 0.05% ammonium hydroxide) to afford methyl 1-(1-morpholinopropan-2-yl)-1H-pyrazole-5-carboxylate (300 mg, 1.18 mmol, 34.4% yield) as a yellow oil. ¹H NMR (400 MHz, DMS₀-d₆): δ 7.52 (d, J=1.6 Hz, 1H), 6.81 (d, J=2.0 Hz, 1H), 5.64-5.59 (m, 1H), 3.87 (s, 3H), 3.60-3.55 (m, 4H), 2.78-2.60 (m, 1H), 2.59-2.50 (m, 1H), 2.49-2.30 (m, 2H), 2.29-2.25 (m, 2H), 1.52 (d, J=6.8 Hz, 3H). LCMS [M+H]⁺=254.0 m/z.

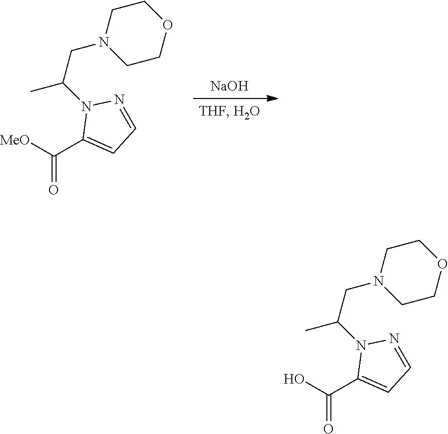

1-(1-morpholinopropan-2-yl)-1H-pyrazole-5-carboxylic acid. To a solution of methyl 1-(1-morpholinopropan-2-yl)-1H-pyrazole-5-carboxylate (300 mg, 1.18 mmol, 1.0 Meq) in THF (5.00 mL) was added NaOH (1.00 M, 2.37 mL, 2.00 eq). The mixture was stirred at RT for 12 h. The reaction mixture was concentrated under reduced pressure to afford 1-(1-morpholinopropan-2-yl)-1H-pyrazole-5-carboxylic acid (280 mg, 1.17 mmol, 98.8% yield, sodium salt) was obtained as a colorless oil.

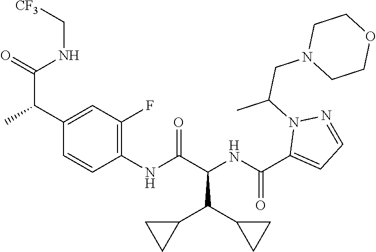 [SMILES: CC(CN1CCOCC1)n1nccc1C(=O)N[C@H](C(=O)Nc1ccc([C@H](C)C(=O)NCC(F)(F)F)cc1F)C(C1CC1)C1CC1]

N-((S)-1,1-dicyclopropyl-3-((2-fluoro-4-((S)-1-oxo-1-((2,2,2-trifluoroethyl)amino)propan-2-yl)phenyl)amino)-3-oxopropan-2-yl)-1-(1-morpholinopropan-2-yl)-1H-pyrazole-5-carboxamide (92). Prepared according to General Scheme A, employing (S)-2-(3-fluoro-4-nitrophenvl)propanoic acid and 2,2,2-trifluoroethan-1-amine hydrochloride followed by (S)-2-((tert-butoxycarbonyl)amino)-3,3-dicyclopropylpropanoic acid and 1-(1-morpholinopropan-2-yl)-1H-pyrazole-5-carboxylic acid. The title compound was isolated as the first eluting, single stereoisomer by chiral SFC purification (column: DAICEL CHIRALPAK AD (250 mm×30 mm, 10 um): mobile phase: 15% [0. % NH₃H₂O in IPA]). LCMS [M+H]⁺=637.4 m/z.

### Example 95: Synthesis of Compound 93

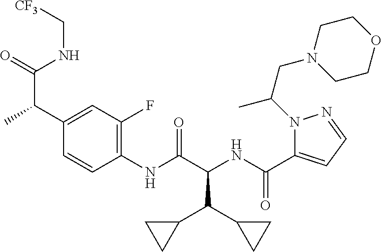 [SMILES: CC(CN1CCOCC1)n1nccc1C(=O)N[C@H](C(=O)Nc1ccc([C@H](C)C(=O)NCC(F)(F)F)cc1F)C(C1CC1)C1CC1]

N-((S)-1,1-dicyclopropyl-3-((2-fluoro-4-((S)-1-oxo-1-((2,2,2-trifluoroethyl)amino)propan-2-yl)phenyl)amino)-3-oxopropan-2-yl)-1-(1-morpholinopropan-2-yl)-1H-pyrazole-5-carboxamide (93). Prepared according to General Scheme A, employing (S)-2-(3-fluoro-4-nitrophenyl)propanoic acid and 2,2,2-trifluoroethan-1-amine hydrochloride followed by (S)-2-((tert-butoxycarbonyl)amino)-3,3-dicyclopropylpropanoic acid and 1-(1-morpholinopropan-2-yl)-1H-pyrazole-5-carboxylic acid. The title compound was isolated as the second eluting, single stereoisomer by chiral SFC purification (column: DAICEL CHIRALPAK AD (250 mm×30 mm, 10 um); mobile phase: 15% 10.1% NH₃H₂O in IPAJ). LCMS [M+H]⁺=637.4 m/z.

### Example 96: Synthesis of Compound 94

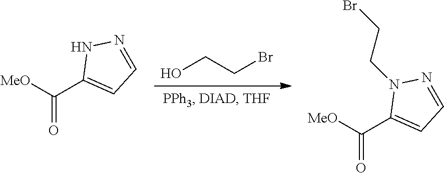

Methyl 1-(2-bromoethyl)-1H-pyrazole-5-carboxylate. To a solution of methyl 1H-pyrazole-5-carboxylate (1.00 g, 7.93 mmol, 1.00 eq), 2-bromoethan-1-ol (1.49 g, 11.9 mmol, 845 μL, 1.50 eq), and PPh₃ (3.12 g, 11.9 mmol, 1.50 eq) in THF (20 mL) was added drop-wise D1AD (2.41 g, 11.9 mmol, 2.31 mL, 1.50 eq) at 0° C., then the mixture was stirred at RT for 2 h. The residue was diluted with H₂O and extracted with ethyl acetate. The combined organic layers were washed with brine, dried over Na₂SO₄, filtered, and concentrated under reduced pressure to give a residue, which was purified by column chromatography (SiO₂, Petroleum ether: EtOAc=100:1 to 1:1) to afford methyl 1-(2-bromoethyl)-1H-pyrazole-5-carboxylate (1.30 g, 5.58 mmol, 70.3% yield) as yellow oil. ¹H NMR (400 MHz, CDCl₃): δ 7.54 (d, J=2.0 Hz, 1H), 6.87 (d, J=2.0 Hz, 1H), 4.97 (t, J=6.8 Hz, 2H), 3.90 (s, 3H), 3.72 (t, J=6.8 Hz, 2H). LCMS [M+H]⁺=234.9 m/z.

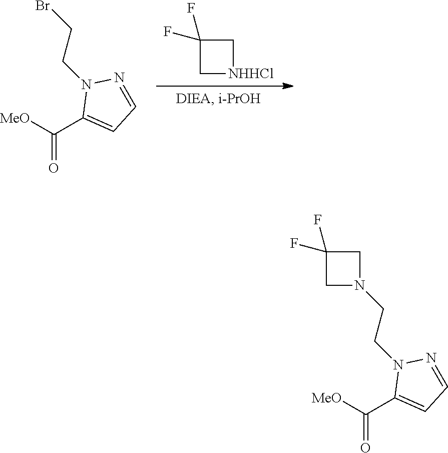

Methyl 1-(2-(3,3-difluoroazetidin-1-yl)ethyl)-1H-pyrazole-5-carboxylate. Methyl 1-(2-bromoethyl)-1H-pyrazole-5-carboxylate (100 mg, 429 umol, l.00 eq), 3,3-difluoroazetidine hydrochloride (11 mg, 858 umol, 2.00 eq), and DIEA (277 mg, 2.15 mmol, 374 uL, 5.00 eq) were taken up into a microwave reaction vial in IPA (3.00 mL). The sealed tube was heated at 100° C. for 8 h under microwave heating. The mixture was concentrated under reduced pressure to afford a residue, which was purified by prep-TLC (SiO₂, Plate 1, Petroleum ether:EtOAc=3:1) to afford methyl 1-(2-(3,3-difluoroazetidin-1-yl)ethyl)-1H-pyrazole-5-carboxylate (90.0 mg, 367 umol, 85.5% yield) as a colorless oil. LCMS [M+H]⁺=246.0 m/z.

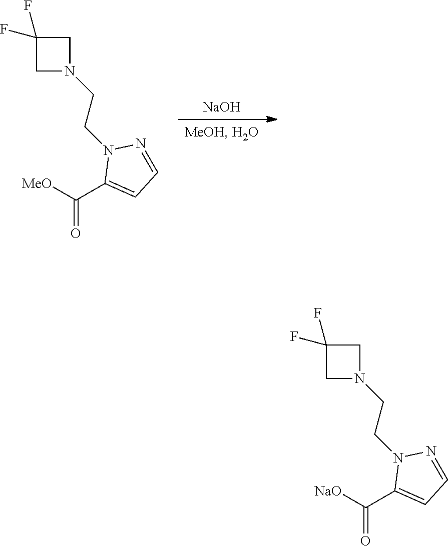

Sodium 1-(2-(1H-imidazol-1-yl)ethyl)-1H-pyrazole-5-carboxylate. To a solution of methyl 1-(2-(3,3-difluoroazetidin-1-yl)ethyl)-1H-pyrazole-5-carboxylate (90.0 mg, 367 umol, 1.00 eq) in MeOH (2.00 mL) was added drop-wise a solution of NaOH (17.6 mg, 440 umol, 1.20 eq) in H₂O (1 00 mL) at 0° C., then the mixture was stirred at RT for 2 h. The mixture was concentrated under reduced pressure to afford sodium 1-(2-(1H-imidazol-1-yl)ethyl)-1H-pyrazole-5-carboxylate (90.0 mg, 354 umol, 96.5% yield, sodium salt) as a white solid.

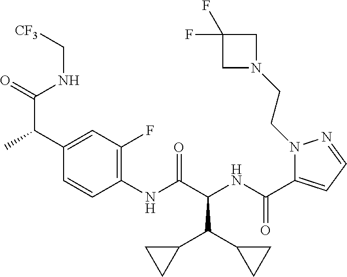 [SMILES: C[C@H](C(=O)NCC(F)(F)F)c1ccc(NC(=O)[C@@H](NC(=O)c2ccnn2CCN2CC(F)(F)C2)C(C2CC2)C2CC2)c(F)c1]

N-((S)-1.1-dicyclopropyl-3-((2-fluoro-4-((S)-1-oxo-1-((2,2,2-trifluoroethyl)amino)propan-2-yl)phenyl)amino)-3-oxopropan-2-yl)-1-(2-(3,3-difluoroazetidin-1-yl)ethyl)-1H-pyrazole-5-carboxamide (94). Prepared according to General Scheme A, employing (S)-2-(3-fluoro-4-nitrophenyl)propanoic acid and 2,2,2-trifluoroethan-1-amine hydrochloride followed by (S)-2-((tert-butoyvcarbonyl)amino)-3,3-dicyclopropylpropanoic acid and sodium 1-(2-(1H-imidazol-1-yl)ethyl)-1H-pyrazole-5-carboxylate. LCMS [M+Hj =629.3 m,

### Example 97: Synthesis of Compound 95

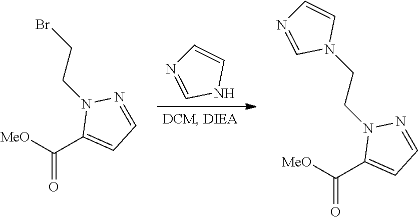

Methyl 1-(2-(1H-imidazol-1-yl)ethyl)-1H-pyrazole-5-carboxylate. methyl 1-(2-bromoethyl)-1H-pyrazole-5-carboxylate (300 mg, 1.29 mmol, 1.00 eq), imidazole (175 mg, 2.57 mmol, 2.00 eq), and K₂CO₃ (534 mg, 3.86 mmol, 3.00 eq) were diluted with 3.0 mL ACN in a microwave reaction vial. The sealed tube was heated at 100° C. for 4 h under microwave heating. The reaction mixture was filtered, concentrated under reduced pressure, and purified by prep-HPLC (eluting with 1040% ACN in water with 10 mM ammonium carbonate) to afford methyl 1-(2-(1H-imidazol-1-yl)ethyl)-1H-pyrazole-5-carboxylate (120 mg, 545 umol, 42.3% yield) as a colorless oil.

LCMS [M+H]⁺=221.1 m/z.

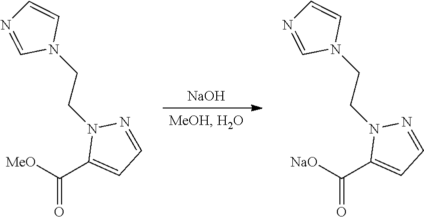

Sodium 1-(2-(1H-imidazol-1-yl)ethyl)-1H-pyrazole-5-carboxylate. To a solution of methyl 1-(2-(1H-imidazol-1-yl)ethyl)-1H-pyrazole-5-carboxylate (100 mg, 454 umol, 1.00 eq) in MeOH (2.00 mL) was added drop-wise a solution of NaOH (18.2 mg, 454 umol, 1.00 eq) in H₂O (1 mL) at 0° C., then the mixture was stirred at RT for 2 h. The mixture was concentrated under reduced pressure to afford crude sodium 1-(2-(1H-imidazol-1-yl)ethyl)-1H-pyrazole-5-carboxylate give a residue (0.100 g, 436 umol, 9%.1% yield, sodium salt) as a white solid.

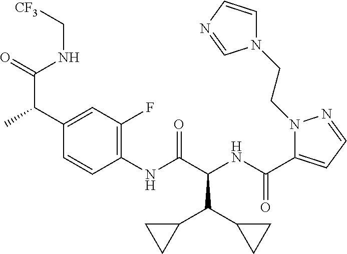 [SMILES: C[C@H](C(=O)NCC(F)(F)F)c1ccc(NC(=O)[C@@H](NC(=O)c2ccnn2CCn2ccnc2)C(C2CC2)C2CC2)c(F)c1]

-(2-(1H-imidazol-1-yl)ethyl)-N-((S)-1,1-dicyclopropyl-3-((2-fluoro-4-((S)-1-oxo-1-((2,2,2-trifluoroethyl)amino)propan-2-yl)phenyl)amino)-3-oxopropan-2-yl)-1H-pyrazole-5-carboxamide (95). Prepared according to General Scheme A, employing (S)-2-(3-fluoro-4-nitrophenyl)propanoic acid and 2,2,2-trifluoroethan-1-amine hydrochloride followed by (S)-2-((tert-butoxycarbonyl)amino)-3,3-dicyclopropylpropanoic acid and sodium 1-(2-(1H-imidazol-1-yl)ethyl)-1H-pyrazole-5-carboxylate. LCMS [M+H]⁺=604.3 m/z.

### Example 98: Synthesis of Compound %

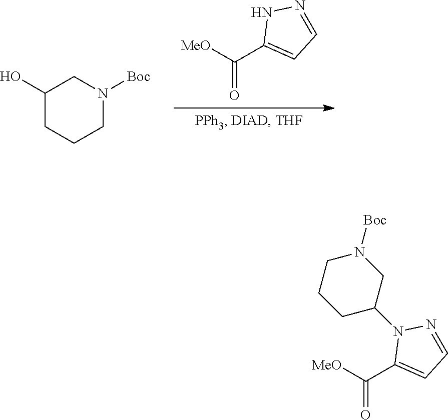

Tert-butyl 3-(5-(methoxycarbonyl)-1H-pyrazol-1-yl)piperidine-1-carboxylate. To a solution of tert-butyl 3-hydroxypiperidine-1-carboxylate (760 mg, 3.78 mmol, 1.5 eq), methyl 1H-pyrazole-5-carboxylate (317 mg, 2.51 mmol, 1 eq), and PPh₃ (988.0 mg, 3.77 mmol, 1.5 eq) in THF (10 mL) was added D1AD (763 mg, 3.77 mmol, 733.65 uL, 1.5 eq) dropwise at 0° C., then the mixture was stirred at RT for 3 h. The reaction was quenched with water and extracted with EtOAc.

The combined organic layers were washed with brine, dried over sodium sulfate, filtered, concentrated under reduced pressure, and purified by column chromatography (SiO₂, eluting with 0-10% EtOAc in petroleum ether) to afford tert-butyl 3-(5-(methoxycarbonyl)-1H-pyrazol-1-yl)piperidine-1-carboxylate (718 mg, 2.32 mmol, 92.43% yield) as a colorless oil. ¹H NMR (400 MHz, CDCl₃): δ 7.51 (d. J=2.0 Hz, 1H), 6.85 (d, J=2.0 Hz, 1H), 5.19-5.12 (m, 1H), 4.26 (brs, 1H), 4.13-4.07 (m, 1H), 3.89 (s, 3H), 3.23 (t, J=11.2 Hz, 1H), 2.78 (t, J=12.4 Hz, 1H), 2.15-2.06 (m, 2H), 1.86-1.83 (m, 1H), 1.67-1.64 (m, 11H), 1.46 (s, 9H).

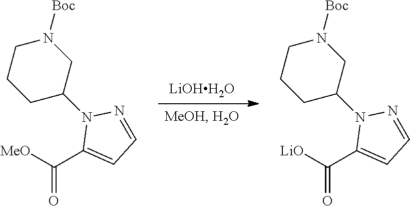

lithium 1-(1-(tert-butoxycarbonyl)piperidin-3-yl)-1H-pyrazole-5-carboxylate. To a solution of tert-butyl 3-(5-(methoxycarbonyl)-1H-pyrazol-1-yl)piperidine-1-carboxylate (718 mg, 2.32 mmol, 1 eq) in THF (10 mL), MeOH (6 mL) and H₂O (2 mL) was added LiOH—H₂O (292.18 mg, 6.96 mmol, 3 eq). The mixture was stirred at 60° C. for 6 h. The reaction mixture was concentrated under reduced pressure to afford crude lithium 1-(1-(tert-butoxycarbonyl)piperidin-3-yl)-1H-pyrazole-5-carboxylate (518 mg, 1.71 mmol, 73.84% yield, lithium salt) as a white foam.

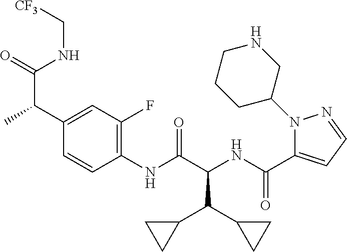 [SMILES: C[C@H](C(=O)NCC(F)(F)F)c1ccc(NC(=O)[C@@H](NC(=O)c2ccnn2C2CCCNC2)C(C2CC2)C2CC2)c(F)c1]

N-((S)-1,1-dicyclopropyl-3-((2-fluoro-4-((S)-1-oxo-1-((2,2,2-trifluoroethyl)amino)propan-2-yl)phenyl)amino)-3-oxopropan-2-yl)-1-(piperidin-3-yl)-1H-pyrazole-5-carboxamide (96). Prepared according to General Scheme A, employing (S)-2-(3-fluoro-4-nitrophenyl)propanoic acid and 2,2,2-trifluoroethan-1-amine hydrochloride followed by (S)-2-((tert-butoxycarbonyl)amino)-3,3-dicyclopropylpropanoic acid and sodium 1-(piperidin-3-yl)-1H-pyrazole-5-carboxylate. The title compound was isolated after SFC separation and hydrochloric acid-mediated Boc removal as follows: tert-butyl 3-(5-(((S)-1,1-dicyclopropyl-3-((2-fluoro-4-((S)-1-oxo-1-((2,2,2-trifluoroethyl)amino)propan-2-yl)phenyl)amino)-3-oxopropan-2-yl)carbamoyl)-1H-pyrazol-1-yl)piperidine-1-carboxylate was separated by chiral SFC purification (column: DAICEL CHIRALPAK AC (250 mm×30 mm, 5 um), mobile phase: 20% [0.1% NH₃H₂O in IPA]) to afford single stereoisomers. The first eluting, single stereoisomer (28 mg, 40 umol, 1.0 eq) was diluted in 0.5 mL DCM and treated with 4 M HCl in dioxane (100 uL, 1 Oeq). the mixture was stirred at RT for 0.5 h. The reaction was treated with saturated sodium bicarbonate and extracted with DCM. The combined organic layers were combined, washed with brine, dried over sodium sulfate, filtered, and concentrated under reduced pressure to afford N-((S)-1,1-dicyclopropyl-3-((2-fluoro-4-((S)-1-oxo-1-((2,2,2-trifluoroethyl)amino)propan-2-yl)phenyl)amino)-3-oxopropan-2-yl)-1-(piperidin-3-yl)-1H-pyrazole-5-carboxamide (20 mg, 33 umol, 81% yield) as a white solid. LCMS [M+H]⁺=593.3 m % z.

### Example 99: Synthesis of Compound 97

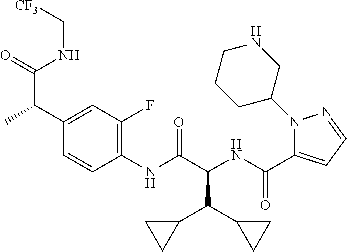 [SMILES: C[C@H](C(=O)NCC(F)(F)F)c1ccc(NC(=O)[C@@H](NC(=O)c2ccnn2C2CCCNC2)C(C2CC2)C2CC2)c(F)c1]

N-((S)-1,1-dicyclopropyl-3-((2-fluoro-4-((5)-1-oxo-1-((2,2,2-triluoroethyl)amino)propan-2-yl)phenyl)amino)-3-oxopropan-2-yl)-1-(piperidin-3-yl)-1H-pyrazole-5-carboxamide (97). The title compound was prepared from the second eluting, single stereoisomer of tert-butyl 3-(5-(((S)-1,1-dicyclopropyl-3-((2-fluoro-4-((S)-1-oxo-1-((2,2,2-trifluoroethyl)amino)propan-2-yl)phenyl)amino)-3-oxopropan-2-yl)carbamoyl)-1H-pyrazol-1-yl)piperidine-1-carboxylate(29 mg, 41.86 umol, 1 eq), which was diluted in 0.5 mL DCM and treated with 4 M HCl in dioxane (100 uL, 1 Oeq). the mixture was stirred at RT for 0.5 h. The reaction was treated with saturated sodium bicarbonate and extracted with DCM. The combined organic layers were combined, washed with brine, dried over sodium sulfate, filtered, and concentrated under reduced pressure to afford N-((S)-1,1-dicyclopropyl-3-((2-fluoro-4-((S)-1-oxo-1-((2,2,2-trifluoroethyl)amino)propan-2-yl)phenyl)amino)-3-oxopropan-2-yl)-1-(piperidin-3-yl)-1H-pyrazole-5-carboxamide (18 mg, 29 umol, 69% yield) as a white solid. LCMS [M+H]⁺=593.3 rtz.

### Example 100: Synthesis of Compound 98

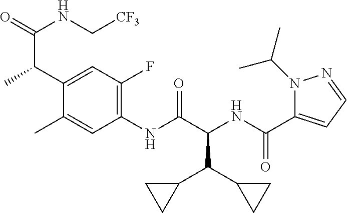 [SMILES: Cc1cc(NC(=O)[C@@H](NC(=O)c2ccnn2C(C)C)C(C2CC2)C2CC2)c(F)cc1[C@H](C)C(=O)NCC(F)(F)F]

N-((S)-1,1-dicyclopropyl-3-((2-fluoro-5-methyl-4-((S)-1-oxo-1-((2,2,2-trifluoroethyl)amino)propan-2-yl)phenyl)amino)-3-oxopropan-2-yl)-1-isopropyl-1H-pyrazole-5-carboxamide (98). Prepared according to General Scheme A, employing 2-(5-fluoro-2-methyl-4-nitrophenyl)propanoic acid and 2,2,2-trifluoroethan-1-amine followed by (S)-2-((tert-butoxycarbonyl)amino)-3,3-dicyclopropylpropanoic acid and 1-isopropyl-1H-pyrazole-5-carboxylic acid. The title compound was isolated as the first eluting, single stereoisomer by chiral SFC purification (column: DAICEL CHIRALPAK OD-H (250 mm×30 mm, 5 um); mobile phase: 20% 10.1% NNH₃H₂O in MeOH]). LCMS [M+H]⁺=566.3 m/z. Absolute configuration was confirmed by resynthesis starting with enantiopure (S)-2-(5-fluoro-2-methyl-4-nitrophenyl)propanoic acid.

### Example 101: Synthesis of Compound 99

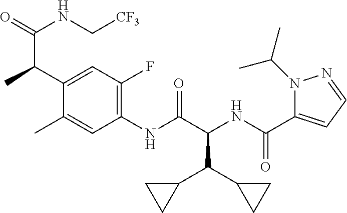 [SMILES: Cc1cc(NC(=O)[C@@H](NC(=O)c2ccnn2C(C)C)C(C2CC2)C2CC2)c(F)cc1[C@@H](C)C(=O)NCC(F)(F)F]

N-((S)-1,1-dicyclopropyl-3-((2-fluoro-5-methyl-4-((R)-1-oxo-1-((2,2,2-trifluoroethyl)amino)propan-2-yl)phenyl)amino)-3-oxopropan-2-yl)-1-isopropyl-1H-pyrazole-5-carboxamide (99). Prepared according to General Scheme A, employing 2-(5-fluoro-2-methyl-4-nitrophenyl)propanoic acid and 2,2,2-trifluoroethan-1-amine followed by (S)-2-((tert-butoxycarbonyl)amino)-3,3-dicyclopropylpropanoic acid and 1-isopropyl-1H-pyrazole-5-carboxylic acid. The title compound was isolated as the second eluting, single stereoisomer by chiral SFC purification (column: DAICEL CHIRALPAK OD-H (250 mm×30 mm, 5 um): mobile phase: 20% [0.1% NH₃H₂O in MeOH]). LCMS [M+H]=566.3 m/,z. Absolute configuration was confirmed by comparison with Example 17252, which was resynthesized starting with enantiopure (S)-2-(5-fluoro-2-methyl-4-nitrophenyl)propanoic acid.

### Example 102: Synthesis of Compound 100

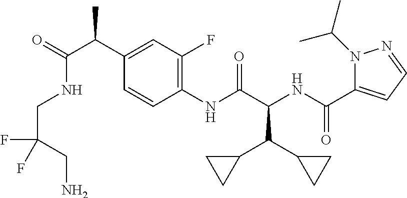 [SMILES: CC(C)n1nccc1C(=O)N[C@H](C(=O)Nc1ccc([C@H](C)C(=O)NCC(F)(F)CN)cc1F)C(C1CC1)C1CC1]

N-((S)-1-((4-((S)-1-((3-amino-2,2-difluoropropyl)amino)-1-oxopropan-2-yl)-2-fluorophenyl)amino)-3,3-dicyclopropyl-1-oxopropan-2-yl)-1-isopropyl-1H-pyrazole-5-carboxamide (100). Prepared using General Procedure A. employing tert-butyl N-13-1[(2S)-2-[4-[[(2S)-3,3-dicyclopropyl1-2-1(2-isopropylpyrazole-3-carbonyl)amino]propanoyl]aminol-3-fluoro-phenyl]propanoyl]amino]-2,2-difluoro-propyl]carbamate. LCMS [M+H]⁺=563.2 m/z.

### Example 103: Synthesis of Compound 101

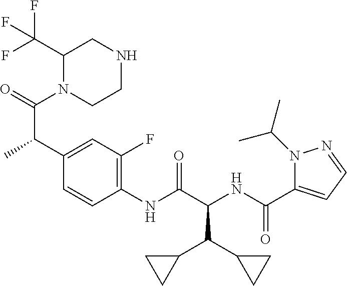 [SMILES: CC(C)n1nccc1C(=O)N[C@H](C(=O)Nc1ccc([C@H](C)C(=O)N2CCNCC2C(F)(F)F)cc1F)C(C1CC1)C1CC1]

N-((2S)-1,1-dicyclopropyl-3-((2-fluoro-4-((2S)-1-oxo-1-(2-(trifluoromethyl)piperazin-1-yl)propan-2-yl)phenyl)amino)-3-oxopropan-2-yl)-1-isopropyl-1 H-pyrazole-5-carboxamide (101).

Prepared using General Procedure A, employing tert-butyl 4-[(2S)-2-[4-[[(2S)-3,3-dicyclopropyl-2-[(2-isopropylpyrazole-3-carbonyl)amino]propanoyllamino]-3-fluoro-phenylpropanoyl]-3-(trifluoromethyl)piperazine-1-carboxylate. LCMS [M+H]⁺=607.2 m/z.

### Example 104: General Scheme B-Synthesis of Compounds 102-105

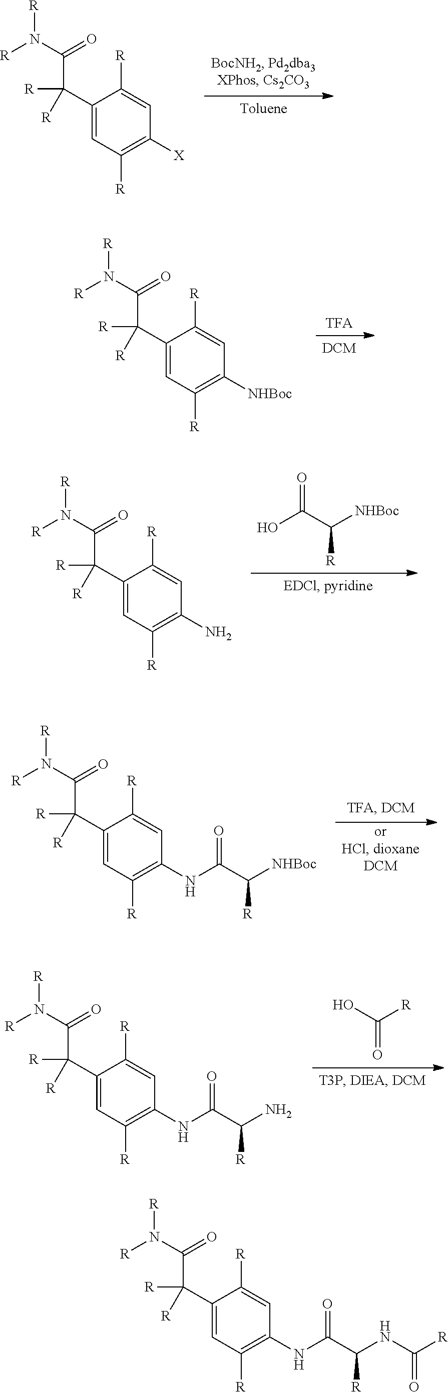

Examples synthesized according to General Scheme B employ an aryl halide in step 1, an amino acid in step three, and a carboxylic acid in step 5. Starting materials are listed in order of use.

### Example 105: Synthesis of 102

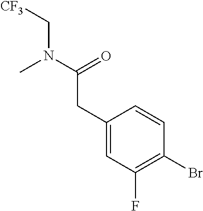 [SMILES: CN(CC(F)(F)F)C(=O)Cc1ccc(Br)c(F)c1]

Synthesis of 2-(4-bromo-3-fluorophenyl)-N-methyl-N-(2,2,2-trifluoroethyl)acetamide. Prepared according to General Procedure B. employing 2-(4-bromo-3-fluorophenyl)acetic acid and 2,2,2-trifluoro-N-methylethan-amine hydrochloride.

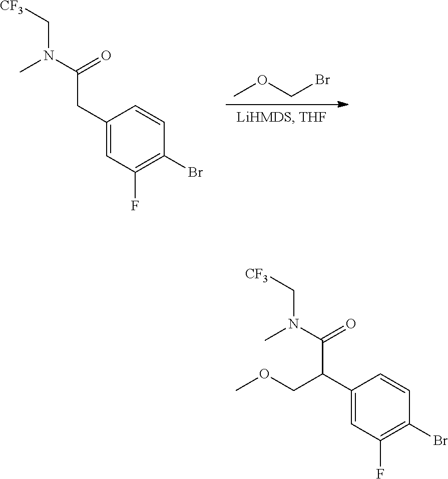

Synthesis of 2-(4-bromo-3-fluorophenyl)-3-methoxy-N-methyl-N-(2,2,2-trifluoroethyl)propanamide. To a solution of 2-(4-bromo-3-fluorophenyl)-N-methyl-N-(2,2,2-trifluoroethyl)acetamide (1.00 g, 3.05 mmol, 1.00 eq) in THF (13.0 mL) was added LiHMDS (1.00 M, 3.05 mL, 1.00 eq) and bromo(methoxy)methane (380 mg, 3.05 mmol, 248 μL, 1.00 eq) at 0′° C.

The mixture was warmed to RT stirred for 2 h. The reaction mixture was diluted with H₂O and extracted with EtOAc, dried over Na₂SO₄, filtered, and concentrated under reduced pressure to give a residue, which was purified by reverse phase prep-HPLC (41-71% CAN in water with 10 mM N₄HCO₃) to afford 2-(4-bromo-3-fluorophenyl)-3-methoxy-N-methyl-N-(2,2,2-trifluoroethyl)propanamide (600 mg, 1.6 mmol, 53% yield) as a yellow oil. ¹H NMR (400 MHz, CDCl₃): δ 7.56-7.48 (m, 1H), 7.19-7.09 (m, 1H), 7.02-6.96 (m, 1H), 4.29-4.17 (m, 1H), 4.09-4.02 (m, 1H), 4.00-3.94 (m, 1H), 3.93-3.83 (m, 1H), 3.57-3.49 (m, 1H), 3.35-3.31 (m, 3H), 3.08-3.03 (m, 3H). LCMS [M+H]⁺=372.0 m/z.

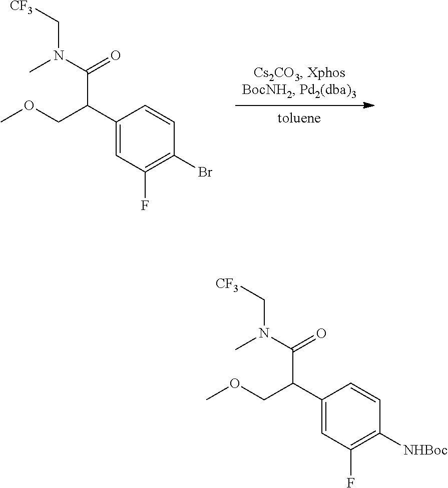 [SMILES: COCC(C(=O)N(C)CC(F)(F)F)c1ccc(Br)c(F)c1 ; "Cs2CO3, XphosBocNH2, Pd2(dba)3" ; "toluene" ; COCC(C(=O)N(C)CC(F)(F)F)c1ccc(NC(=O)OC(C)(C)C)c(F)c1]

Synthesis of tert-butyl (2-fluoro-4-(3-methoxy-1-(methyl(2,2,2-trifluoroethyl)amino)-1-oxopropan-2-yl)phenyl)carbamate. A mixture of 2-(4-bromo-3-fluorophenyl)-3-methoxy-N-methyl-N-(2,2,2-trifluoroethyl)propanamide (560 mg, 1.50 mmol, 1.00 eq), tert-butyl carbamate (264 mg, 2.26 mmol, 1.50 eq), Pd₂(dba)₃ (68.9 mg, 75.2 umol, 0.05 eq), C⁵2CO₃ (1.23 g, 3.76 mmol, 2.5 eq), and Xphos (71.7 mg, 150 umol, 0.100 eq) in toluene (15.0 mL) was de-gassed and then heated to 80° C. for 2 h under N₂. The reaction mixture was filtered and concentrated under reduced pressure to give a residue, which was purified by silica gel column chromatography (PE:EtOAc=100: 1 to 3:1) to afford tert-butyl (2-fluoro-4-(3-methoxy-1-(methyl(2,2,2-trifluoroethyl)amino)-1-oxopropan-2-yl)phenyl)carbamate (400 mg, 980 umol, 65% yield) as a yellow oil. LCMS [M+HI =409.1 m/z.

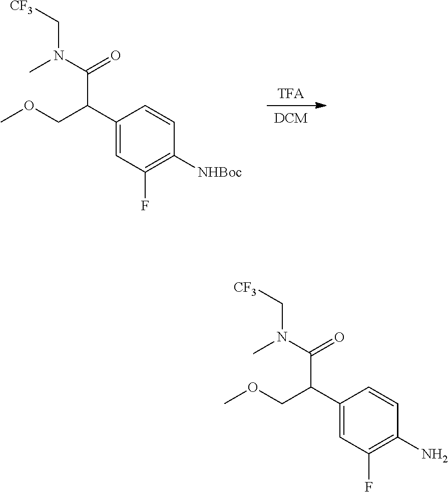

Synthesis of 2-(4-amino-3-fluorophenyl)-3-methoxy-N-methyl-N-(2,2,2-trifluoroethyl)propanamide. Prepared according to General Procedure A, employing tert-butyl (2-fluoro-4-(3-methoxy-1-(methyl (2,2,2-trifluoroethyl)amino)-1-oxopropan-2-yl)phenyl)carbamate.

¹H NMR (400 MHz, CDCl₃): δ 7.01-6.91 (m, 1H), 6.89-6.83 (m, 1H), 6.79-6.71 (m, 1H), 4.30-4.17 (m, 1H), 3.98-3.81 (m, 3H), 3.50-3.45 (m, 1H), 3.35-3.32 (m, 3H), 3.06-3.03 (m, 3H).

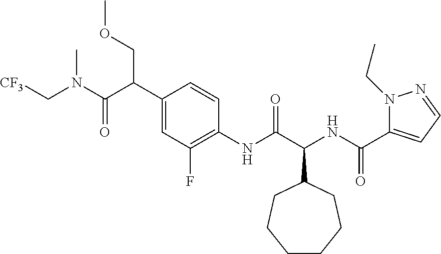 [SMILES: CCn1nccc1C(=O)N[C@H](C(=O)Nc1ccc(C(COC)C(=O)N(C)CC(F)(F)F)cc1F)C1CCCCCC1]

Synthesis of N—((I S)-1-cycloheptyl-2-((2-fluoro-4-(3-methoxy-1-(methyl(2,2,2-trifluoroethyl)amino)-1-oxopropan-2-yl)phenyl)amino)-2-oxoethyl)-1-ethyl-1H-pyrazole-5-carboxamide (102). Prepared according to General Scheme B, employing 2-(4-amino-3-fluorophenyl)-3-methoxy-N-methyl-N-(2,2,2-trifluoroethyl)propanamide, (S)-2-((tert-butoxycarbonyl)amino)-2-cycloheptylacetic acid, and 1-ethyl-1H-pyrazole-5-carboxylic acid. The title compound was isolated as a white solid, single stereoisomer, and first eluting, single stereoisomer by chiral SFC purification (column: DAICEL CHIRALPAK AD (250 mm×30 mm, 10 um); mobile phase: 25% [0.1% NH₃H₂O in IPA]). LCMS [M+H]⁺=584.3 m,-Example 106: Synthesis of Compound 103

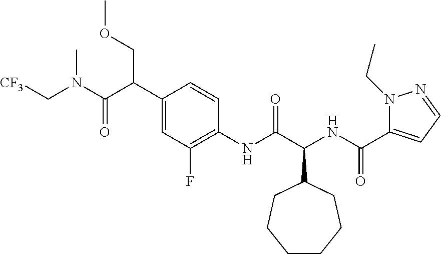 [SMILES: CCn1nccc1C(=O)N[C@H](C(=O)Nc1ccc(C(COC)C(=O)N(C)CC(F)(F)F)cc1F)C1CCCCCC1]

Synthesis of N-((1S)-1-cycloheptyl-2-((2-fluoro-4-(3-methoxy-1-(methyl(2,2,2-trifluoroethyl)amino)-1-oxopropan-2-yl)phenyl)amino)-2-oxoethyl)-1-ethyl-1H-pyrazole-5-carboxamide (103). Prepared according to General Scheme B, employing 2-(4-amino-3-fluorophenyl)-3-methoxy-N-methyl-N-(2,2,2-trifluoroethyl)propanamide, (S)-2-((tert-butoxycarbonyl)amino)-2-cycloheptylacetic acid, and 1-ethyl-1H-pyrazole-5-carboxylic acid. The title compound was isolated as a white solid, single stereoisomer, and second eluting, single stereoisomer by chiral SFC purification (column: DAICEL CHIRALPAK AD (250 mm×30 mm, 10 um); mobile phase: 25% 10.1% NH₃H₂O in IPA|). LCMS [M+H]⁺=584.3 m/z

### Example 107: Synthesis of Compound 104

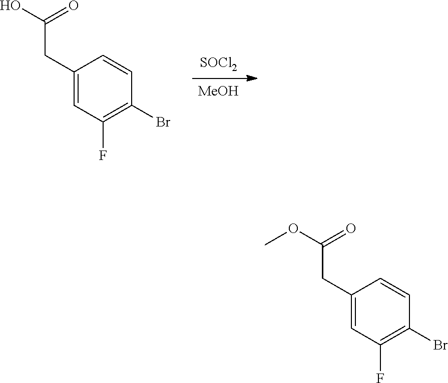

methyl 2-(4-bromo-3-fluorophenyl)acetate. To a solution of 2-(4-bromo-3GP-322,C₃ fluorophenyl)acetic acid (3.00 g, 12.8 mmol, 1.00 eq) in MeOH (30.0 mL) was added SOC12 (3.06 g, 25.8 mmol, 1.87 mL, 2.00 eq) and DMF (94.1 mg, 1.29 mmol, 99.1 uL, 0.100 eq) at 0° C. The mixture was stirred at RT for 1 h. The reaction mixture was concentrated under reduced pressure to give a residue, which was diluted with H₂O. The mixture was basified with sat. aqueous NaHCO₃ to pH=8 and extracted with EtOAc, dried over Na₂SO₄, filtered, and concentrated in vacuo to afford methyl 2-(4-bromo-3-fluorophenyl)acetate (3.10 g, 12.6 mmol, 97.4% yield) as a yellow oil.

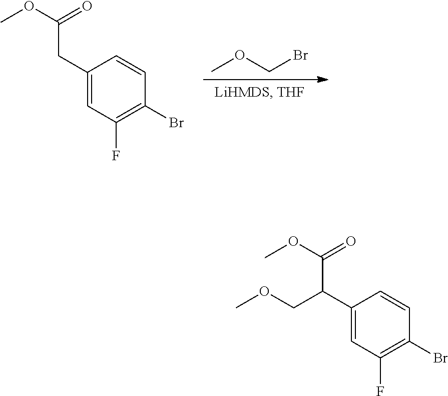

methyl 2-(4-bromo-3-fluorophenyl)-3-methoxypropanoate. To a solution of methyl 2-(4-bromo-3-fluorophenyl)acetate (3.10 g, 12.5 mmol, 1.00 eq) in THF (50.0 mL) was added LiHMDS (12.5 mL, 1.00 eq) and bromo(methoxy)methane (1.57 g, 12.6 mmol, 1.02 mL, 1.00 eq) at −15° C. The mixture was stirred at −15° C. for 1 h. The mixture was diluted with H₂O, acidified with 1 M HCl to pH=8.0, and extracted with EtOAc, dried over Na₂SO₄, filtered, and concentrated in vacuo. The resulting residue was purified by column chromatography (SiO₂, Petroleum ether:EtOAc=1:0 to 100:3) to afford methyl 2-(4-bromo-3-fluorophenyl)-3-methoxypropanoate (3.30 g, 11.3 mmol, 90.3% yield) as colourless oil. ¹H NMR (400 MHz, CDCl₃): δ 7.52-7.48 (m, 1H), 7.20-7.11 (m, 1H), 7.05-6.95 (m, 1H), 3.95-3.75 (m, 2H), 3.71 (s, 3H), 3.68-3.57 (m, 1H), 3.36 (s, 3H).

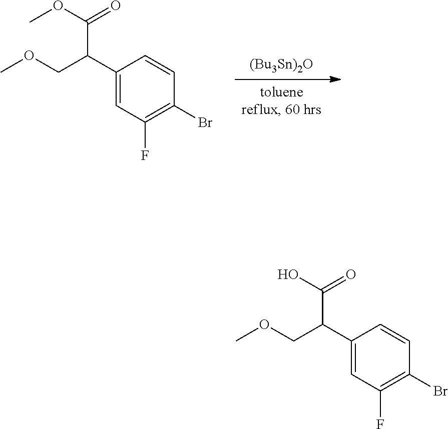 [SMILES: "(Bu3Sn)2O" ; COCC(C(=O)OC)c1ccc(Br)c(F)c1 ; "toluenereflux, 60 hrs" ; COCC(C(=O)O)c1ccc(Br)c(F)c1]

-(4-bromo-3-fluorophenyl)-3-methoxypropanoic acid. To a solution of methyl 2-(4-bromo-3-fluorophenyl)-3-methoxypropanoate (1.00 g, 3.44 mmol, 1.00 eq) in toluene (20.0 mL) was added (Bu₃Sn)₂O (9.36 g, 15.7 mmol, 8.00 mL, 4.57 eq) at RT. The mixture was stirred at 120° C. for 60 h. The mixture was filtered, concentrated under reduced pressure, diluted with 10 mL sat. aq. KF and 30.0 mL H₂O, and stirred at RT for 2 h. The mixture was acidified with 1.0 M HCl to pH=5.0, filtered, extracted with DCM, dried over Na₂SO₄, filtered, and concentrated under reduced pressure. The resulting residue was purified by prep-HPLC (eluting with 1-30% EtOH in hexanes with 0.1% formic acid) to afford 2-(4-bromo-3-fluorophenyl)-3-methoxypropanoic acid (800 mg, 2.89 mmol, 84.1% yield) as a colorless oil. ¹H NMR (400 MHz, CDCl₃): δ 7.51 (t, J=3.8 Hz, 1H), 7.24-7.13 (m, 1H), 7.08-6.95 (m, 1H), 3.95-3.80 (m, 2H), 3.70-3.60 (m, 1H), 3.45-3.30 (m, 3H). LCMS [2M+H]⁺=552.9 m/z.

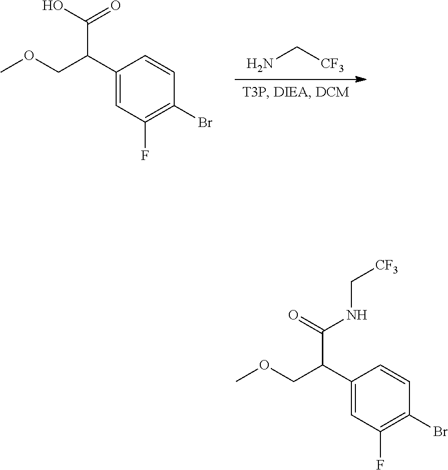

2-(4-bromo-3-fluorophenyl)-3-methoxy-N-(2,2,2-trifluoroethyl)propanamide. To a solution of 2-(4-bromo-3-fluorophenyl)-3-methoxypropanoic acid (400 mg, 1.44 mmol, 1.00 eq) and 2,2,2-trifluoroethan-1-amine (214 mg, 2.17 mmol, 170 μL, 1.50 eq) in DCM (10.0 mL) was added T3P (1.38 g, 2.17 mmol, 1.29 mL, 50% purity, 1.50 eq) and DIEA (1.87 g, 14.4 mmol, 2.51 mL, 10.0 eq). The mixture was stirred at 0° C. for 1 h. The reaction mixture was diluted with H₂O and extracted with EtOAc. The combined organic layers were washed with sat. NaCl, dried over Na₂SO₄, filtered, and concentrated under reduced pressure to afford crude 2-(4-bromo-3-fluorophenyl)-3-methoxy-N-(2,2,2-trifluoroethyl)propanamide (500 mg, 1.40 mmol, 96.7% yield) as a yellow solid. LCMS [M+H]⁺=357.9 m/z.

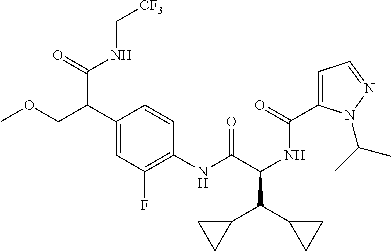 [SMILES: COCC(C(=O)NCC(F)(F)F)c1ccc(NC(=O)[C@@H](NC(=O)c2ccnn2C(C)C)C(C2CC2)C2CC2)c(F)c1]

N-((2S)-1.1-dicyclopropyl-3-((2-fluoro-4-(3-methoxy-1-oxo-1-((2,2,2-trifluoroethyl)amino)propan-2-yl)phenyl)amino)-3-oxopropan-2-yl)-1-isopropyl-1H-pyrazole-5-carboxamide (104). Prepared according to General Scheme B. employing 2-(4-bromo-3-fluorophenyl)-3-methoxy-N-(2,2,2-trifluoroethyl)propanamide, (S)-2-((tert-butoxycarbonyl)amino)-3,3-dicyclopropylpropanoic acid, and 1-isopropyl-1H-pyrazole-5-carboxylic acid. The title compound was isolated as the first eluting, single stereoisomer by chiral SFC purification (column: DAICEL CHIRALPAK OD—H (250 mm×30 mm, 5 um): mobile phase: 20% 10.1% NH₁₋₁₃H₂O in MeOH]). LCMS [M+H]⁺=582.3 m/z.

### Example 108: Synthesis of Compound 105

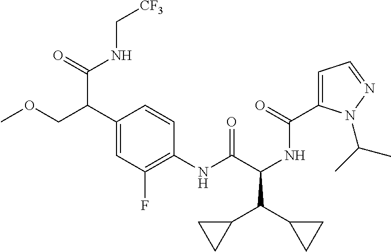 [SMILES: COCC(C(=O)NCC(F)(F)F)c1ccc(NC(=O)[C@@H](NC(=O)c2ccnn2C(C)C)C(C2CC2)C2CC2)c(F)c1]

N-((2S)-1,1-dicyclopropyl-3-((2-fluoro-4-(3-methoxy-1-oxo-1-((2,2,2-trifluoroethyl)amino)propan-2-yl)phenyl)amino)-3-oxopropan-2-yl)-1-isopropyl-1H-pyrazole-5-carboxamide (105). Prepared according to General Scheme B, employing 2-(4-bromo-3-fluorophenyl)-3-methoxy-N-(2,2,2-trifluoroethyl)propanamide, (S)-2-((tert-butoxycarbonyl)amino)-3,3-dicyclopropylpropanoic acid, and 1-isopropyl-1H-pyrazole-5-carboxylic acid. The title compound was isolated as the second eluting, single stereoisomer by chiral SFC purification (column: DAICEL CHIRALPAK OD—H (250 mm×30 mm, 5 um); mobile phase: 20% 10.1% NH₃H₂O in MeOH]). LCMS [M+H]⁺=582.3 m/z.

### Example 109: General Scheme E-Synthesis of Compound 106

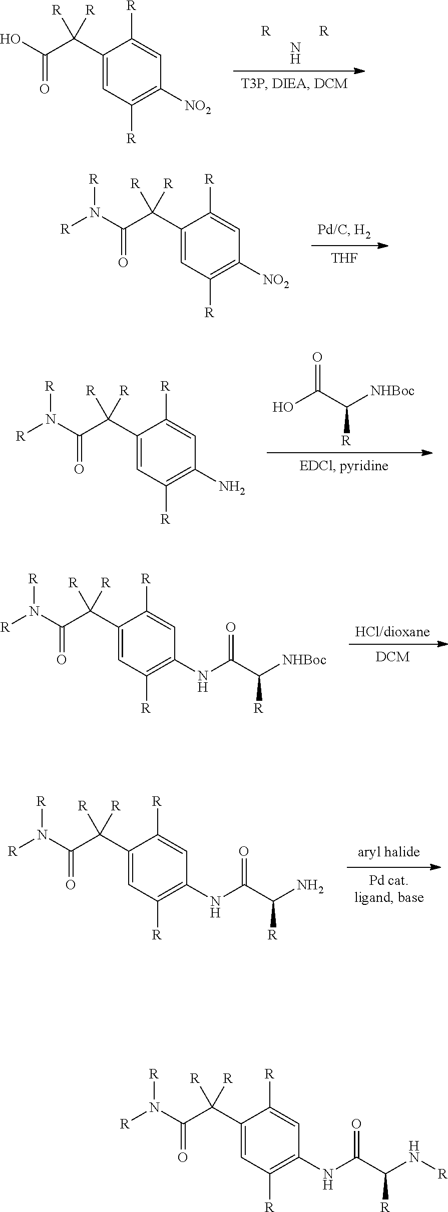

### Example 110: Synthesis of Compound 106

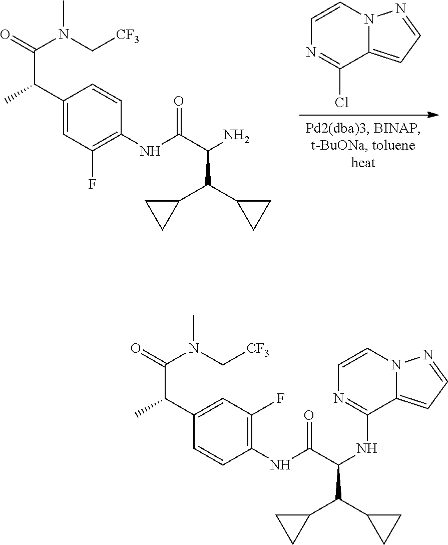 [SMILES: C[C@H](C(=O)N(C)CC(F)(F)F)c1ccc(NC(=O)[C@@H](N)C(C2CC2)C2CC2)c(F)c1 ; Clc1nccn2nccc12 ; "Pd2(dba)3, BINAP,t-BuONa, tolueneheat" ; C[C@H](C(=O)N(C)CC(F)(F)F)c1ccc(NC(=O)[C@@H](Nc2nccn3nccc23)C(C2CC2)C2CC2)c(F)c1]

(S)-3,3-dicyclopropyl-N-(2-fluoro-4-((S)-1-(methyl(2,2,2-trifluoroethyl)amino)-1-oxopropan-2-yl)phenyl)-2-(pyrazolo[1,5-a]pyrazin-4-ylamino)propenamide (106). Prepared according to General Scheme E, employing (S)-2-(3-fluoro-4-nitrophenyl)propanoic acid and 2,2,2-trifluoro-N-methylethan-1-amine hydrochloride, then (S)-2-((tert-butoxycarbonyl)amino)-3,3-dicyclopropylpropanoic acid and 4-chloropyrazolol 1,5-a]pyrazine, as exemplified in the following procedure: A mixture of (S)-2-amino-3,3-dicyclopropyl-N-(2-fluoro-4-((S)-1-(methyl(2,2,2-trifluoroethyl)amino)-1-oxopropan-2-yl)phenyl)propanamide (35.0 mg, 81.5 umol, 1.00 eq), 4-chloropyrazolol1,5-a]pyrazine (15.0 mg, 97.8 umol, 1.20 eq), BINAP (5.07 mg, 8.15 umol, 0.100 eq), Pd₂(dba)₃ (7.46 mg, 8.15 umol, 100 eq) and t-BuONa (11.7 mg, 122 umol, 1.50 eq) in toluene (1.00 mL) was stirred at 100° C. for 3 h. The reaction mixture was filtered and concentrated under reduced pressure to give a residue, which was purified by prep-TLC (SiO₂, petroleum ether:EtOAc 1:1) and then prep-HPLC, eluting with 46-76% ACN in water with 0.225% formic acid, to afford (S)-3,3-dicyclopropyl-N-(2-fluoro-4-((S)-1-(methyl(2,2,2-trifluoroethyl)amino)-1-oxopropan-2-yl)phenyl)-2-(pyrazolo[1,5-a]pyrazin-4-ylamino)propanamide (15.0 mg, 27.3 umol, 33.5% yield) as a white solid. LCMS [M+H]+=547.3.

### Example 111: General Scheme F-Synthesis of Compound 107

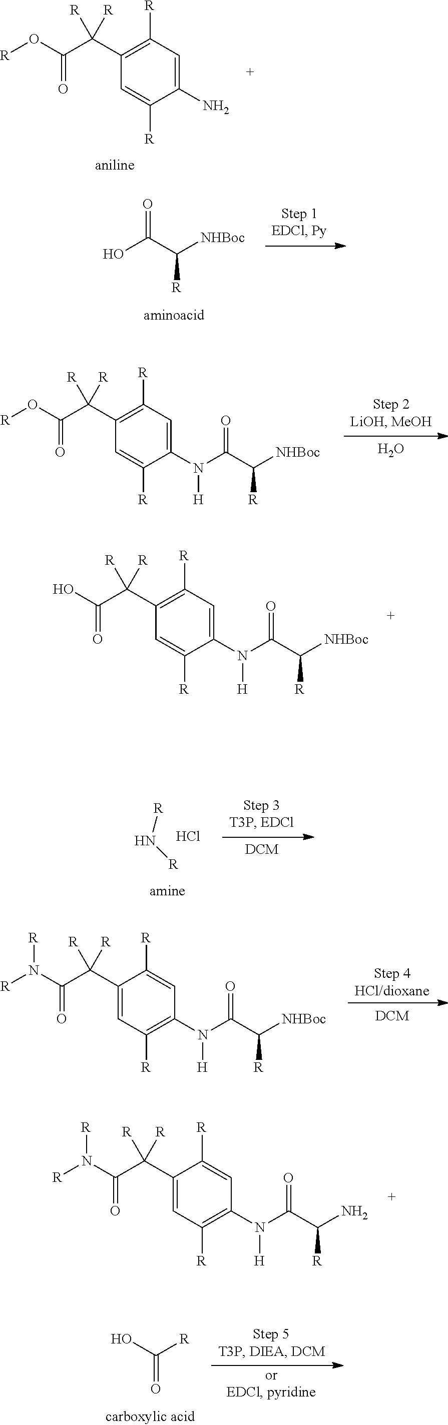

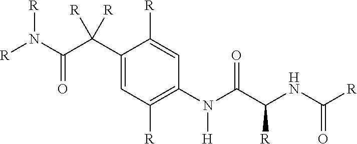

Examples synthesized according to General Scheme F employ an aniline and amino acid in step one followed by an amine in step 3 and a carboxylic acid in step 5. Starting materials are listed in order of use.

### Example 112: Synthesis of Compound 107

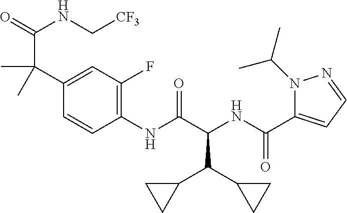 [SMILES: CC(C)n1nccc1C(=O)N[C@H](C(=O)Nc1ccc(C(C)(C)C(=O)NCC(F)(F)F)cc1F)C(C1CC1)C1CC1]

(S)-N-(1.1-dicyclopropyl-3-((2-fluoro-4-(2-methyl-1-oxo-1-((2,2,2-trifluoroethyl)amino)propan-2-yl)phenyl)amino)-3-oxopropan-2-yl)-1-isopropyl-1H-pyrazole-5-carboxamide (107). Prepared according to General Scheme F, employing ethyl 2-(4-amino-3-fluorophenyl)-2-methylpropanoate and (S)-2-((tert-butoxycarbonyl)amino)-3.3-dicyclopropylpropanoic acid followed by 2,2,2-trifluoroethan-1-amine hydrochloride and 1-isopropyl-1H-pyrazole-5-carboxylic acid. LCMS [M+H]⁺=566.3 m.

### Example 113 General Scheme D—Synthesis of Compounds 108-114

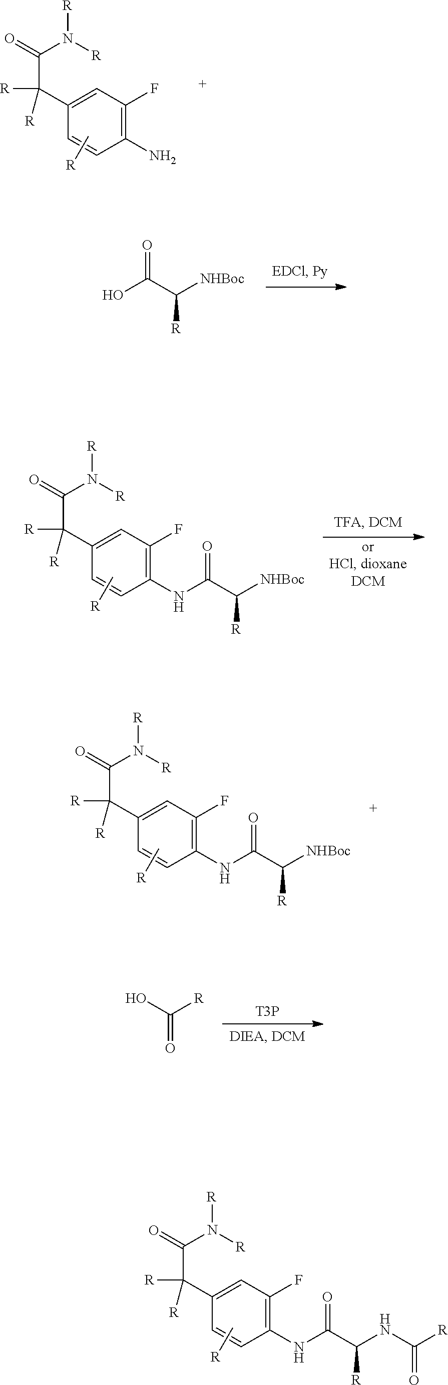

Examples synthesized according to General Scheme D employ an aryl amine and an amino acid in the first step followed by a carboxylic acid in step 3. Starting materials are listed in order of use.

### Example 114: Synthesis of Compound 108

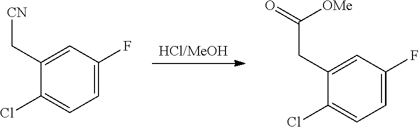 [SMILES: N#CCc1cc(F)ccc1Cl ; "HCl/MeOH" ; COC(=O)Cc1cc(F)ccc1Cl]

methyl 2-(2-chloro-5-fluorophenyl)acetate. A solution of 2-(2-chloro-5-fluorophenyl)acetonitrile (2.50 g, 14.7 mmol, 1.0 Xeq) in HCl/MeOH (4.00 M, 36.9 mL, 10.0 eq) was stirred at 80° C. for 12 h. The mixture was concentrated under reduced pressure, diluted with H₂O, basified with sat. aqueous NaHCO₃ to pH=8.00, and extracted with EtOAc, dried over anhydrous Na₂SO₄, filtered, and concentrated under reduced pressure to afford methyl 2-(2-chloro-5-fluorophenyl)acetate (2.70 g, 13.3 mmol, 90.4% yield) as a colorless oil. ¹H NMR (400 MHz, CDCl₃): δ 7.40-7.30 (m, 1H), 7.12-7.05 (m, 1H), 6.98-6.87 (m, 1H), 3.76 (s, 2H), 3.73 (s, 3H).

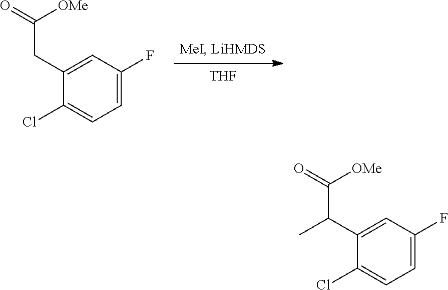

methyl 2-(2-chloro-5-fluorophenyl)propanoate. To a solution of methyl 2-(2-chloro-5-fluorophenyl)acetate (2.70 g, 13.3 mmol, 1.00 eq) in THF (30.0 mL) was added LiHMDS (1.00 M, 13.3 mL, 1 eq) and Mel (1.89 g, 13.3 mmol, 830 μL, 1.00 eq). The mixture was stirred at 0° C. for 1 h. The mixture was diluted with H₂O and extracted with EtOAc, dried over anhydrous Na₂SO₄, filtered, and concentrated under reduced pressure to afford methyl 2-(2-chloro-5-fluorophenyl)propanoate (2.75 g, 12.7 mmol, 95.2% yield) as a yellow oil. ¹H NMR (40 MHz, CDCl₃): δ 7.40-7.30 (m, 1H), 7.11-7.04 (m, 1H), 6.98-6.87 (m, 1H), 4.19 (q, J=7.2 Hz, 1H), 3.70 (s, 3H), 1.50 (d, J=7.2 Hz, 3H).

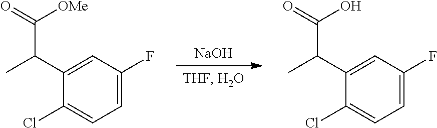

2-(2-chloro-5-fluorophenyl)propanoic acid. To a solution of methyl 2-(2-chloro-5-fluorophenyl)propanoate (2.75 g, 12.7 mmol, 1.00 eq) in THF (20.0 mL) was added NaOH (1 00 M, 25.4 mL, 2.00 eq). The mixture was stirred at RT for 12 h. The mixture was diluted with H₂O and extracted with EtOAc. The aqueous layer was acidified with 1.0 M HCl to pH=4.0, extracted with EtOAc, dried over anhydrous Na₂SO₄, filtered, and concentrated under reduced pressure to afford 2-(2-chloro-5-fluorophenyl)propanoic acid (2.40 g, 11.9 mmol, 93.3% yield) as a yellow solid. ¹H NMR (400 MHz, CDCl₃): δ 7.39-7.32 (m, 1H), 7.14-7.07 (m, 1H), 6.98-6.92 (m, 1H), 4.24 (q, J =6.8 Hz, 1H), 1.54 (d, J=7.2 Hz, 3H).

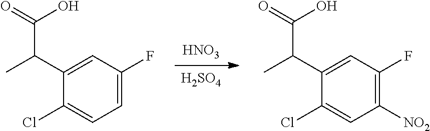

2-(2-chloro-5-fluoro-4-nitrophenyl)propanoic acid. To a solution of 2-(2-chloro-5-fluorophenyl)propanoic acid (2.40 g, 11.9 mmol, 1.00 eq) in H₂SO₄ (20.0 mL) was added HNO₃ (1.60 g, 17.8 mmol, 1.14 mL, 70.0% purity, 1.50 eq). The mixture was stirred at 0° C. for 1 h. The reaction mixture was diluted to H₂O, extracted with EtOAc, dried over Na₂SO₄, filtered, and concentrated under reduced pressure to afford 2-(2-chloro-5-fluoro-4-nitrophenyl)propanoic acid (2.70 g, 10.9 mmol, 92.0% yield) as a yellow solid. ¹H NMR (400 MHz, CDCl₃): δ 8.18-8.11 (d, J=6.8 Hz, 1H), 7.39-7.31 (m, 1H), 4.29 (q J 7.2 Hz, 1H), 1.60 (d J=7.2 Hz, 3H).

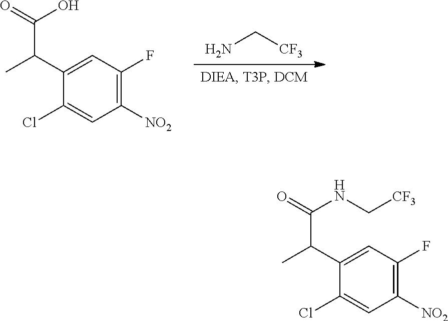

2-(2-chloro-5-fluoro-4-nitrophenyl)-N-(2,2,2-trifluoroethyl)propanamide. Prepared according to General Procedure B, employing 2-(2-chloro-5-fluoro-4-nitrophenyl)propanoic acid and 2,2,2-trifluoroethan-1-amine. LCMS [M+H]⁺=329.0 m/z.

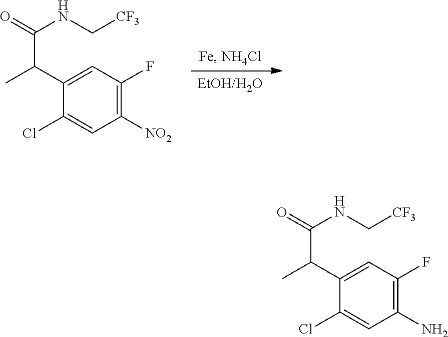

2-(4-amino-2-chloro-5-N₁fuorophenyl)-N-(2,2,2-trifluoroethyl)propanamide. To a solution of 2-(2-chloro-5-fluoro-4-nitrophenyl)-N-(2,2,2-trifluoroethyl)propananide (660 mg, 2.01 mmol, 1.00 eq) in H₂O (3.00 mL) and EtOH (8.00 mL) was added Fe (561 mg, 10.0 mmol, 5.00 eq) and NH₄C₁ (537 mg, 10.0 mmol, 5.00 eq). The mixture was stirred at 80° C. for 1 h. The reaction mixture was filtered. the filtrate was treated with sat. aq. NaJHCO3 and then was filtered again. The filtrate was concentrated under reduced pressure to give a residue. The residue was diluted with H₂O, extracted with EtOAc, dried over anhydrous Na₂SO₄, filtered, and concentrated under reduced pressure to afford 2-(4-amino-2-chloro-5-fluorophenyl)-N-(2,2,2-trifluoroethyl)propanamide (590 mg, 1.98 mmol, 98.3% yield) as a yellow solid. LCMS [M+H]⁺=299.1 m/z.

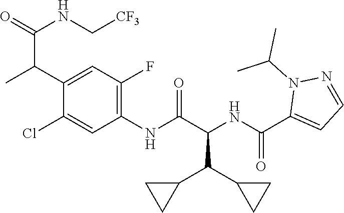 [SMILES: CC(C(=O)NCC(F)(F)F)c1cc(F)c(NC(=O)[C@@H](NC(=O)c2ccnn2C(C)C)C(C2CC2)C2CC2)cc1Cl]

N-((2S)-1-((5-chloro-2-fluoro-4-(1-oxo-1-((2,2,2-trifluoroethyl)amino)propan-2-yl)phenyl)amino)-3,3-dicyclopropyl-1-oxopropan-2-yl)-1-isopropyl-1H-pyrazole-5-carboxamide (108). Prepared according to General Scheme D, employing 2-(4-amino-2-chloro-5-fluorophenyl)-N-(2,2,2-trifluoroethyl)propanamide and (S)-2-((tert-butoxycarbonyl)amino)-3,3-dicyclopropylpropanoic acid followed by 1-isopropyl-1H-pyrazole-5-carboxylic acid. The title compound was isolated as the first eluting, single stereoisomer by chiral SFC purification (column: DAICEL CHIRALPAK AD-H (250 mm×30 mm, 5 um); mobile phase: 15% [0.1% NH₃H₂O in IPA]). LCMS [M+H]⁺=586.1 m/z.

### Example 115: Synthesis of Compound 109

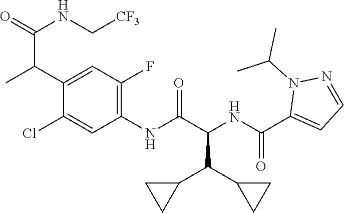 [SMILES: CC(C(=O)NCC(F)(F)F)c1cc(F)c(NC(=O)[C@@H](NC(=O)c2ccnn2C(C)C)C(C2CC2)C2CC2)cc1Cl]

N-((2S)-1-((5-chloro-2-fluoro-4-(1-oxo-1-((2,2,2-trifluoroethyl)amino)propan-2-yl)phenyl)amino)-3,3-dicyclopropyl-1-oxopropan-2-yl)-1-isopropyl-1H-pyrazole-5-carboxamide (109). The title compound was isolated as the second eluting, single stereoisomer by chiral SFC purification (column: DAICEL CHIRALPAK AD-H (250 mm×30 mm, 5 urn): mobile phase: 15% NH₃H₂O in IPA]). LCMS [M+H]⁺=586.1 m % z.

### Example 116: Synthesis of Compound 110

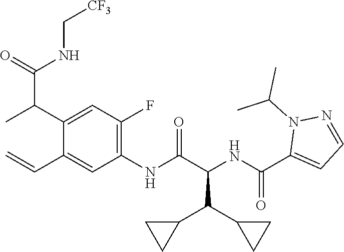 [SMILES: C=Cc1cc(NC(=O)[C@@H](NC(=O)c2ccnn2C(C)C)C(C2CC2)C2CC2)c(F)cc1C(C)C(=O)NCC(F)(F)F]

N-((2S)-1,1-dicyclopropyl-3-((2-fluoro-4-(1-oxo-1-((2,2,2-trifluoroethyl)amino)propan-2-yl)-5-vinylphenyl)amino)-3-oxopropan-2-yl)- 1-isopropyl-1H-pyrazole-5-carboxamide (110). Prepared as a mixture of isomers according to General Scheme D, employing 2-(4-amino-5-fluoro-2-vinylphenyl)-N-(2,2,2-trifluoroethyl)propanamide and (S)-2-((tert-butoxycarbonyl)amino)-3,3-dicyclopropylpropanoic acid followed by 1-isopropyl-1H-pyrazole-5-carboxylic acid. LCMS [M+H]⁺=578.3 m/z.

### Example 117: Synthesis of Compound 111

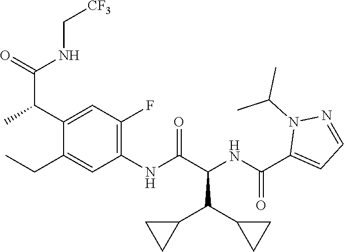 [SMILES: CCc1cc(NC(=O)[C@@H](NC(=O)c2ccnn2C(C)C)C(C2CC2)C2CC2)c(F)cc1[C@H](C)C(=O)NCC(F)(F)F]

N-((S)-1,1-dicyclopropyl-3-((5-ethyl-2-fluoro-4-((S)-1-oxo-1-((2,2,2-trifluoroethyl)amino)propan-2-yl)phenyl)amino)-3-oxopropan-2-yl)-1-isopropyl-1H-pyrazole-5-carboxamide (111). Prepared according to General Scheme D, employing (S)-2-(4-amino-2-ethyl-5-fluorophenyl)-N-(2,2,2-trifluoroethyl)propanamide and (S)-2-((tert-butoxycarbonyl)amino)-3,3-dicyclopropylpropanoic acid followed by 1-isopropyl-1H-pyrazole-5-carboxylic acid. LCMS [M+H]⁺=580.3 m/z.

### Example 118: Synthesis of Compound 112

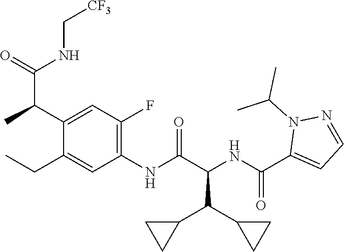 [SMILES: CCc1cc(NC(=O)[C@@H](NC(=O)c2ccnn2C(C)C)C(C2CC2)C2CC2)c(F)cc1[C@@H](C)C(=O)NCC(F)(F)F]

N-((S)-1,1-dicyclopropyl-3-((5-ethyl-2-fluoro-4-((R)-1-oxo-1-((2,2,2-trifluoroethyl)amino)propan-2-yl)phenyl)amino)-3-oxopropan-2-yl)-1-isopropyl-1H-pyrazole-5-carboxamide (112). Prepared according to General Scheme D, employing (R)-2-(4-amino-2-ethyl-5-fluorophenyl)-N-(2,2,2-trifluoroethyl)propanamide and (S)-2-((tert-butoxycarbonyl)amino)-3,3-dicyclopropylpropanoic acid followed by 1-isopropyl-1H-pyrazole-5-carboxylic acid. LCMS [M+H]⁺=580.3 m/z.

### Example 119: Synthesis of Compound 113

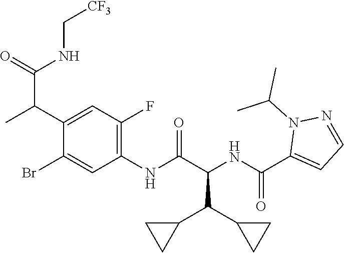 [SMILES: CC(C(=O)NCC(F)(F)F)c1cc(F)c(NC(=O)[C@@H](NC(=O)c2ccnn2C(C)C)C(C2CC2)C2CC2)cc1Br]

N-((2S)-1-((5-bromo-2-fluoro-4-(1-oxo-1-((2,2,2-trifluoroethyl)amino)propan-2-yl)phenyl)amino)-3,3-dicyclopropyl-1-oxopropan-2-yl)-1-isopropyl-1H-pyrazole-5-carboxamide (113). Prepared as a mixture of diastereomers according to General Scheme D, employing 2-(4-amino-2-bromo-5-fluorophenyl)-N-(2,2,2-trifluoroethyl)propanamide and (S)-2-((tert-butoxycarbonyl)amino)-3,3-dicyclopropylpropanoic acid followed by 1-isopropyl-1H-pyrazole-5-carboxylic acid. LCMS [M+H]⁺=632.2 m/z.

### Example 120: Synthesis of Compound 114

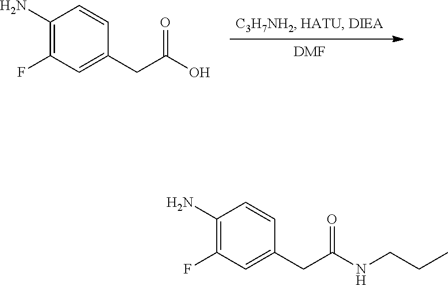

-(4-amino-3-fluorophenyl)-N-propylacetamide. To a solution of 2-(4-amino-3-fluorophenyl)acetic acid (250 mg, 1.48 mmol, 1.00 eq) in DMF (10.0 mL) was added propan-1-amine (500 mg, 8.46 mmol, 695 μL, 5.72 eq), HATU (875 mg, 2.30 mmol, 1.56 eq), and DIEA (928 mg, 7.18 mmol, 1.25 mL, 4.86 eq) at RT. The mixture was stirred at RT for 12 h. The reaction mixture was diluted by H₂O, extracted by EtOAc, washed by H₂O, dried over anhydrous Na₂SO₄, and concentrated under reduced pressure to give a residue. The residue was purified by Prep-TLC (petroleum ether:EtOAc=2:1) to afford 2-(4-amino-3-fluorophenyl)-N-propylacetamide (130 mg, 618 umol, 41.8% yield) as a yellow solid. ¹H NMR (400 MHz, CDCl₃): δ 6.89 (dd, J₁=2.0 Hz, J_{2 =11.6} Hz, 1H), 6.82 (dd, J, =1.6 Hz,12=8.0 Hz, 1H), 6.76 (t, =8.8 Hz, 1H), 5.43 (brs, 1H), 3.75 (brs, 2H), 3.45 (s, 2H), 3.19-3.14 (m, 2H), 1.51-1.40 (m, 2H), 0.85 (t, J=7.2 Hz, 3H).

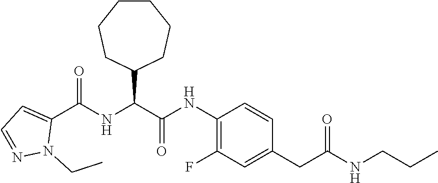 [SMILES: CCCNC(=O)Cc1ccc(NC(=O)[C@@H](NC(=O)c2ccnn2CC)C2CCCCCC2)c(F)c1]

(S)-N-(1-cycloheptyl-2-((2-fluoro-4-(2-oxo-2-(propylamino)ethyl)phenyl)amino)-2-oxoethyl)-1-ethyl-1H-pyrazole-5-carboxamide (114). Prepared according to General Scheme D, employing 2-(4-amino-3-fluorophenyl)-N-propylacetamide and (S)-2-((tert-butoxycarbonyl)amino)-2-cycloheptylacetic acid followed by 1-ethyl-1H-pyrazole-5-carboxylic acid. LCMS [M+H]⁺=486.5 n:.

### Example 121: Synthesis of Compound 115

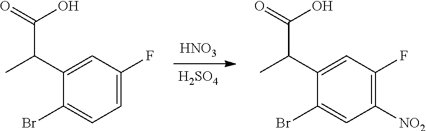

-(2-bromo-5-fluoro-4-nitrophenyl)propanoic acid. To a solution of 2-(2-bromo-5-fluorophenyl)propanoic acid (2.20 g, 8.90 mmol, 1.00 eq) in H₂SO₄ (20.0 mL) was added HNO₃ (1.23 g, 13.7 mmol, 880 μL, 70.0% purity, 1.54 eq) at 0° C. slowly. The resulting mixture was stirred at 0° C. for 2 h. The mixture was diluted with ice water and extracted with EtOAc. The combined organic layers were washed with H₂O, dried over Na₂SO₄, filtered, and concentrated to afford 2-(2-bromo-5-fluoro-4-nitrophenyl)propanoic acid (2.45 g, 8.39 mmol, 94.2% yield) as yellow solid. ¹H NMR (400 MHz, CDCl₃): δ 8.32 (d, J=6.8 Hz, 1H), 7.36 (d, J=11.6 Hz, 1H), 4.30 (q, J=6.8 Hz, 1H), 1.61 (d, J=6.8 Hz, 3H).

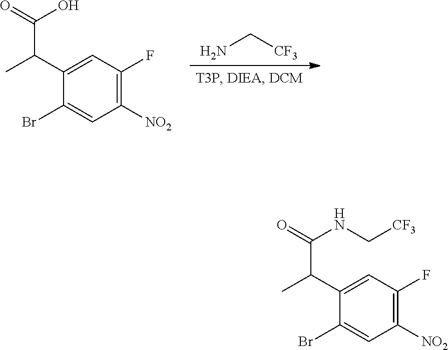

2-(2-bromo-5-fluoro-4-nitrophenyl)-N-(2,2,2-trifluoroethyl)propanamide. To a solution of 2-(2-bromo-5-fluoro-4-nitrophenyl)propanoic acid (2.45 g, 8.39 mmol, 1.00 eq) and 2,2,2-trifluoroethan-1-amine (914 mg, 9.23 mmol, 725 μL, 1.10 eq) in DCM (30.0 mL) was added DIEA (5.42 g, 41.9 mmol, 7.31 mL, 5.00 eq) and T3P (10.7 g, 16.8 mmol, 9.98 mL, 50.0% purity, 2.00 eq) at 0° C. The mixture was stirred at RT for 1 h. The reaction mixture was diluted with H₂O and extracted with EtOAc. The combined organic layers were washed with sat. aq. NH₄C₁, dried over Na₂SO₄, filtered, concentrated under reduced pressure, and purified by prep-HPLC (35-65% ACN in water with 0.225 formic acid) to afford 2-(2-bromo-5-fluoro-4-nitrophenyl)-N-(2,2,2-trifluoroethyl)propanamide (3.05 g, 8.17 mmol, 97.5% yield) as yellow solid. ¹H NMR (400 MHz, CDCl₃): δ 8.31 (d, J=6.8 Hz, 1H), 7.48 (d, J=11.6 Hz, 1H), 5.92 (s, 1H), 4.11 (q, J=6.8 Hz, 1H), 9.97-3.89 (m, 2H), 1.64 (d, J=7.2 Hz, 3H).

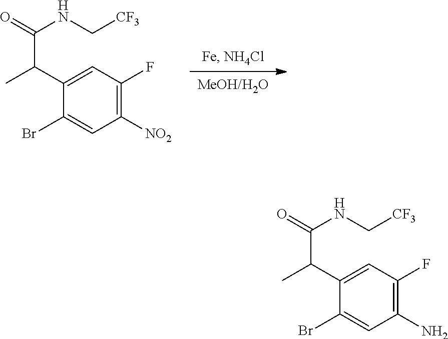

2-(4-amino-2-bromo-5-fluorophenyl)-N-(2,2,2-trifluoroethyl)propanamide. To a solution of 2-(2-bromo-5-fluoro-4-nitrophenyl)-N-(2,2,2-trifluoroethyl)propanamide (1.50 g, 4.02 mmol, 1.00 eq) in H₂O (2.00 mL) and MeOH (20.0 mL) was added Fe (1.12 g, 20.2 mmol, 5.01 eq) and NH₄C₁ (1.12 g, 21.0 mmol, 5.23 eq). The mixture was stirred at 80° C. for 5 h. The mixture was filtered, rinsing with MeOH (50.0 mL), and the filtrate was concentrated to afford 2-(4-amino-2-bromo-5-fluorophenyl)-N-(2,2,2-trifluoroethyl)propanamide (1.30 g, 3.79 mmol, 94.2% yield) as yellow solid. ¹H NMR (400 MHz, DMS₀): δ 8.60-8.56 (m, 1H), 7.02-6.96 (m, 2H), 3.95-3.80 (m, 5H), 1.31-1.27 (m, 3H).

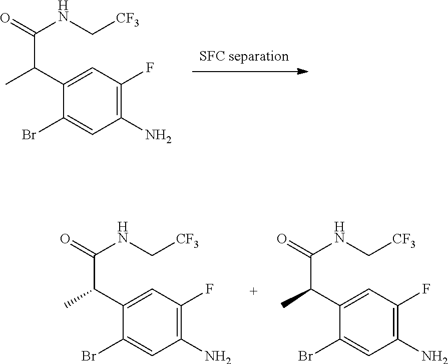 [SMILES: CC(C(=O)NCC(F)(F)F)c1cc(F)c(N)cc1Br ; "SFC separation" ; C[C@H](C(=O)NCC(F)(F)F)c1cc(F)c(N)cc1Br ; C[C@@H](C(=O)NCC(F)(F)F)c1cc(F)c(N)cc1Br]

(S)-2-(4-amino-2-bromo-5-fluorophenyl)-N-(2,2,2-trifluoroethyl)propenamid and (R)-2-(4-amino-2-bromo-5-fluorophenyl)-N-(2,2,2-trifluoroethyl)propenamide. Title compounds were isolated as single stereoisomers by chiral SFC separation (column: REGIS (s,s) WHELK-Ol (250×50 mm, 10 um); mobile phase: 30% [0.1% NH₃H₂O in IPA]). (S)-2-(4-amino-2-bromo-5-fluorophenyl)-N-(2,2,2-trifluoroethyl)propenamide was isolated as the first eluting stereoisomer. ¹H NMR (400 MHz, CDCl₃): δ 7.05-6.98 (m, 2H), 5.75-5.70 (m, 1H), 4.08-3.78 (m, 5H), 1.47 (d. J =7.2 Hz, 3H). LCMS [M+H]⁺=343.0 m,-. (R)-2-(4-amino-2-bromo-5-fluorophenyl)-N-(2,2,2-trifluoroethyl)propenamide was isolated as the second eluting stereoisomer. ¹H NMR (400 MHz, CDCl₃): δ 7.05-6.98 (m, 2H), 5.75-5.70 (m, 1H), 4.09-3.78 (m, 5H), 1.47 (d, J=7.2 Hz, 3H). LCMS [M+H]⁺=343.0 m % z. Absolute configuration was elucidated by single crystal x-ray diffraction of (R)-2-(4-amino-2-bromo-5-fluorophenyl)-N-(2,2,2-trifluoroethyl)propenamide as the acetonitrile adduct, crystals grown by slow evaporation of 2 mg title compound in 0.2 mL of 4:1 ACN/water, [parameters: (CuKα), space group=12, a=15.4985(12), b=4.8592(3), c=21.0027(16), and alpha=90°, beta=103.736(8°), gamma=90° 1.

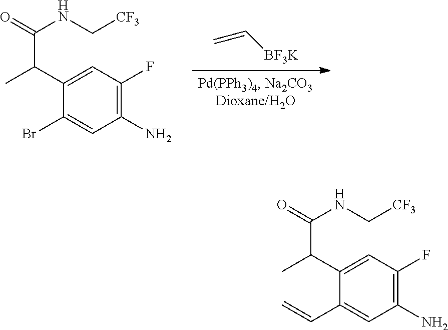

-(4-amino-5-fluoro-2-vinylphenyl)-N-(2,2,2-trifluoroethyl)propanamide. To a mixture of 2-(4-amino-2-bromo-5-fluorophenyl)-N-(2,2,2-trifluoroethyl)propanamide (1.00 g, 2.91 mmol, 1.00 eq), potassium vinyltrifluoroborate (586 mg, 4.37 mmol, 1.50 eq), and Na₂CO₃ (618 mg, 5.83 mmol, 2.00 eq) in dioxane (20.0 mL) and H₂O (5.00 mL) was added Pd(PPh₃)₄(337 mg, 291 umol, 0. 100 eq). The mixture was degassed and purged with N₂ and stirred at 80° C. for 12 h. The reaction mixture was diluted with water and extracted with EtOAc, and the combined organic layers were washed with water and brine, dried over Na₂SO₄, and concentrated under reduced pressure to give a residue, which was purified by column (SiO₂, petroleum ether: EtOAc=1:0 to 10:1) to afford 2-(4-amino-5-fluoro-2-vinylphenyl)-N-(2,2,2-trifluoroethyl)propanamide (380 mg, 1.31 mmol, 44.9% yield) as a white solid. LCMS [M+H]⁺=291.0 m/z.

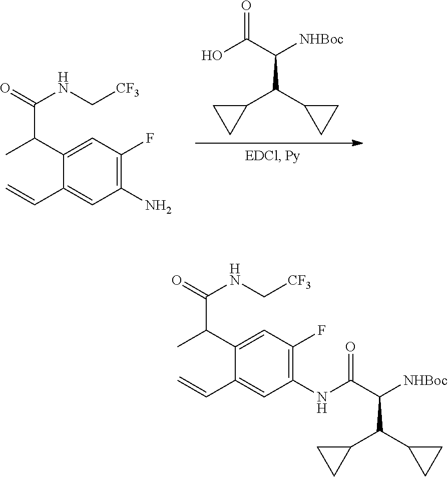 [SMILES: C=Cc1cc(N)c(F)cc1C(C)C(=O)NCC(F)(F)F ; CC(C)(C)OC(=O)N[C@H](C(=O)O)C(C1CC1)C1CC1 ; "EDCl, Py" ; C=Cc1cc(NC(=O)[C@@H](NC(=O)OC(C)(C)C)C(C2CC2)C2CC2)c(F)cc1C(C)C(=O)NCC(F)(F)F]

tert-butyl ((2S)-1,1-dicyclopropyl-3-((2-fluoro-4-(1-oxo-1-((2,2,2-trifluoroethyl)amino)propan-2-yl)-5-vinylphenyl)amino)-3-oxopropan-2-yl)carbamate. To a solution of 2-(4-amino-5-fluoro-2-vinylphenyl)-N-(2,2,2-trifluoroethyl)propanamide (380 mg, 1.31 mmol, 1.00 eq) and (S)-2-((tert-butoxycarbonyl)amino)-3,3-dicyclopropylpropanoic acid (458 mg, 1.70 mmol, 1.30 eq) in pyridine (10.0 mL) was added EDCI (502 mg, 2.62 mmol, 2.00 eq). The reaction mixture was stirred at RT for 12 h. The reaction mixture was concentrated under reduced pressure, diluted with DCM, washed with water and brine, dried over Na₂SO₄, and concentrated under reduced pressure to afford tert-butyl ((2S)-1,1-dicyclopropyl-3-((2-fluoro-4-(1-oxo-1-((2,2,2-trifluoroethyl)amino)propan-2-yl)-5-vinylphenyl)amino)-3-oxopropan-2-yl)carbamate (760 mg, crude) as a yellow solid. LCMS [M+H]⁺=542.4 m Z.

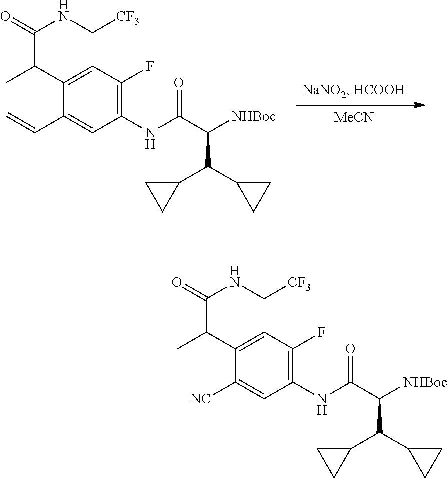

tert-butyl ((2S)-1-((5-cyano-2-fluoro-4-(1-oxo-1-((2,2,2-trifluoroethyl)amino)propan-2-yl)phenyl)amino)-3,3-dicyclopropyl-1-oxopropan-2-yl)carbamate. To a solution of tert-butyl ((2S)-1,1-dicyclopropyl-3-((2-fluoro-4-(1-oxo-1-((2,2,2-trifluoroethyl)amino)propan-2-yl)-5-vinylphenyl)amino)-3-oxopropan-2-yl)carbamate (730 mg, 1.35 mmol, 1.00 eq) and NaNO₂ (465 mg, 6.74 mmol, 5.00 eq) in MeCN (20.0 mL) was added HCOOH (1.62 g, 33.7 mmol, 25.0 eq). The mixture was heated to 70° C. for 12 h. The reaction mixture was concentrated under reduced pressure to give a residue. The residue was diluted with water and extracted with DCM. The combined organic layers were washed with water and brine, dried over Na₂SO₄, concentrated under reduced pressure, and purified by column (SiO₂, petroleum ether: EtOAc=1:0 to 10:1) to afford tert-butyl ((2S)-1-((5-cyano-2-fluoro-4-(1-oxo-1-((2,2,2-trifluoroethyl)amino)propan-2-yl)phenyl)amino)-3,3-dicyclopropyl-1-oxopropan-2-yl)carbamate (180 mg, 333 umol, 24.7% yield) as a yellow solid. LCMS [M+HI]=563.1 m/z.

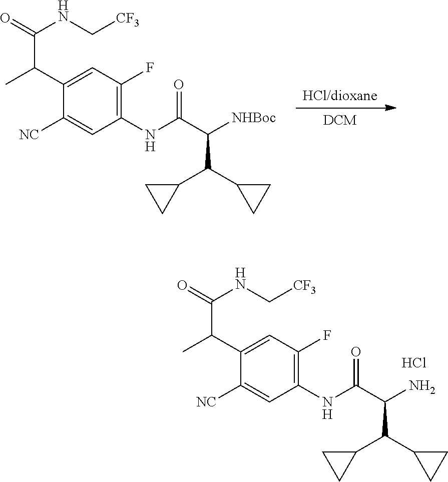

(2S)-2-amino-N-(5-cyano-2-fluoro-4-(1-oxo-1-((2,2,2-trifluoroethyl)amino)propan-2-yl)phenyl)-3,3-dicyclopropylpropanamide hydrochloride. To a solution of tert-butyl ((2S)-1-((5-cyano-2-fluoro-4-(1-oxo-1-((2,2,2-trifluoroethyl)amino)propan-2-yl)phenyl)amino)-3,3-dicyclopropyl-1-oxopropan-2-yl)carbamate (180 mg, 333 umol, 1.00 eq) in DCM (5.00 mL) was added HCl/dioxane (4 M, 5.0 mL, 60 eq) at 0° C. The mixture was warmed to RT and stirred for 10 h. The reaction mixture was concentrated under reduced pressure to afford (2S)-2-amino-N-(5-cyano-2-fluoro-4-(1-oxo-1-((2,2,2-trifluoroethyl)amino)propan-2-yl)phenyl)-3,3-dicyclopropylpropanamide (159 mg, crude, HCl) as a yellow solid. LCMS [M+H]⁺=441. 1i m/z.

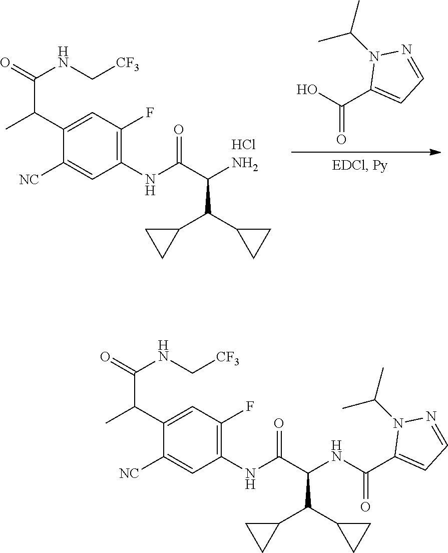

N-((2S)-1-((5-cyano-2-fluoro-4-(1-oxo-1-((2,2,2-trifluoroethyl)amino)propan-2-yl)phenyl)amino)-3,3-dicyclopropyl-1-oxopropan-2-yl)-1-isopropyl-1H-pyrazole-5-carboxamide (115). To a solution of (2S)-2-amino-N-(5-cvano-2-fluoro-4-(1-oxo-1-((2,2,2-trifluoroethyl)amino)propan-2-yl)phenyl)-3,3-dicyclopropylpropanamide (158 mg, 331 umol, 1.00 eq, HCl) and 1-isopropyl-1H-pyrazole-5-carboxylic acid (76.6 mg, 497 umol, 1.50 eq) in pyridine (1.00 mL) was added EDCI (127 mg, 663 umol, 2.00 eq) and stirred at RT for 2 h. The reaction mixture was concentrated under reduced pressure, diluted with water, and extracted with DCM.

The combined organic layers were washed with water and brine, dried over Na₂SO₄, and concentrated under reduced pressure to give a residue. The residue was purified by prep-TLC (2:1 petroleum ether: EtOAc) to afford N-((2S)-1-((5-cyano-2-fluoro-4-(1-oxo-1-((2,2,2-trifluoroethyl)amino)propan-2-yl)phenyl)amino)-3,3-dicyclopropyl-1-oxopropan-2-yl)-1-isopropyl-1H-pyrazole-5-carboxamide (100 mg, 173 umol, 52.4% yield) as a yellow solid. LCMS [M+HI=577.2 m/z. Title compound was isolated as the first eluting, single stereoisomer by chiral SFC purification (column: REGIS (S<S) WHELK-Ol (250×25 mm, 10 um), mobile phase: 15% [0.1% NH₃H₂O in IPA|). LCMS [M+H]⁺=577.2 m/z.

### Example 122: Synthesis of Compound 116

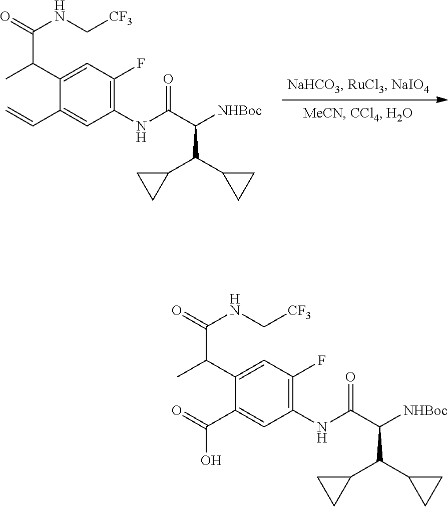

5-((S)-2-((tert-butoxycarbonyl)amino)-3,3-dicyclopropylpropanamido)-4-fluoro-2-(1-oxo-1-((2,2,2-trifluoroethyl)amino)propan-2-yl)benzoic acid. To a solution of tert-butyl ((2S)-1,l-dicyclopropyl-3-((2-fluoro-4-(1-oxo-1-((2,2,2-trifluoroethyl)amino)propan-2-yl)-5-vinylphenyl)amino)-3-oxopropan-2-yl)carbamate (30) mg, 554 umol, 1.00 eq) in ACN (4.00 mL) was added CCl₄ (3.00 mL), H₂O (2.00 mL) and NaHCO₃ (233 mg, 2.77 mmol, 108 μL, 5.00 eq), followed by adding NaIO₄ (948 mg, 4.43 mmol, 246 μL, 8.00 eq) and RuC1₃ (34.5 mg, 166 umol, 11.1 uL, 0.300 eq) at 0° C. The mixture was warmed to RT and stirred for 2 h. The reaction mixture was treated with sat. aq. Na₂SO₃ and adjusted the pH of the solution to 3 with 1 N HCl. The mixture was extracted with DCM, the combined organic layer was washed with water and brine, dried over Na₂SO₄, and concentrated under reduced pressure to give a residue. The residue was purified by column (SiO₂, DCM: MeOH=1:0 to 10:1) to afford 5-((S)-2-((tert-butoyvcarbonyl)amino)-3,3-dicyclopropylpropanamido)-4-fluoro-2-(1-oxo-1-((2,2,2-trifluoroethyl)amino)propan-2-yl)benzoic acid (80.0 mg, 143 umol, 25.8% yield) as a black solid.

LCMS [M+H-Boc]=460.2 m/z.

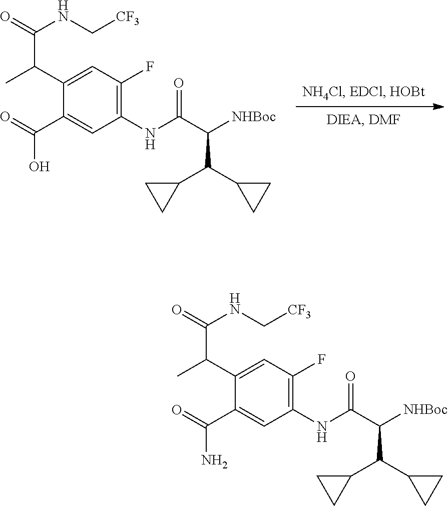 [SMILES: CC(C(=O)NCC(F)(F)F)c1cc(F)c(NC(=O)[C@@H](NC(=O)OC(C)(C)C)C(C2CC2)C2CC2)cc1C(=O)O ; "DIEA, DMF" ; "NH4Cl, EDCl, HOBt" ; CC(C(=O)NCC(F)(F)F)c1cc(F)c(NC(=O)[C@@H](NC(=O)OC(C)(C)C)C(C2CC2)C2CC2)cc1C(N)=O]

tert-butyl ((2S)-1-((5-carbamoyl-2-fluoro-4-(1-oxo-((2,2,2-trifluoroethyl)amino)propan-2-yl)phenyl)amino)-3,3-dicyclopropyl-1-oxopropan-2-yl)carbamate. To a solution of 5-((S)-2-((tert-butoxycarbonyl)amino)-3,3-dicyclopropylpropanamido)-4-fluoro-2-(1-oxo-1-((2,2,2-trifluoroethyl)amino)propan-2-yl)benzoic acid (79.0 mg, 141 umol, 1.00 eq), NH₄Cl (38.0 mg, 710 umol, 5.03 eq), HOBt (38.2 mg, 282 umol, 2.0 Xeq), and DIEA (73.0 mg, 565 umol, 98.4 uL, 4.00 eq) in DMF (1.50 mL) was added EDCI (54.1 mg, 282 umol, 2.00 eq). The reaction mixture was stirred at RT for 12 h. The reaction mixture was diluted with water and extracted with 10:1 EtOAc:DCM. The combined organic layers were washed with water and brine, dried over Na₂SO₄, and concentrated under reduced pressure to give a residue. The residue was purified by prep-TLC (10:1 DCM:MeOH) to afford tert-butyl ((2S)-1-((5-carbamoyl-2-fluoro-4-(1-oxo-1-((2,2,2-trifluoroethyl)amino)propan-2-yl)phenyl)amino)-3,3-dicyclopropyl-1-oxopropan-2-yl)carbamate (50.0 mg, 89.5 umol, 63.4% yield) as a yellow oil. LCMS [M+H]⁺=559.4 m/z.

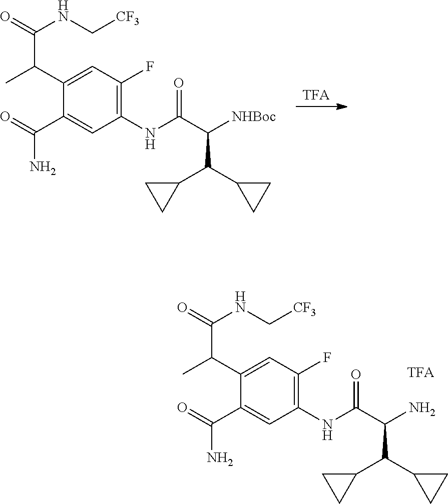 [SMILES: CC(C(=O)NCC(F)(F)F)c1cc(F)c(NC(=O)[C@@H](NC(=O)OC(C)(C)C)C(C2CC2)C2CC2)cc1C(N)=O ; CC(C(=O)NCC(F)(F)F)c1cc(F)c(NC(=O)[C@@H](N)C(C2CC2)C2CC2)cc1C(N)=O ; "TFA" ; "TFA"]

5-((S)-2-amino-3,3-dicyclopropylpropanamido)-4-fluoro-2-(1-oxo-1-((2,2,2-trifluoroethyl)amino)propan-2-yl)benzamide trifluoroacetate. To a solution of tert-butyl ((2S)-1-((5-carbamoyl-2-fluoro-4-(1-oxo-1-((2,2,2-trifluoroethyl)amino)propan-2-yl)phenyl)amino)-3,3-dicyclopropyl-1-oxopropan-2-yl)carbamate (40.0 mg, 71.6 umol, 1.00 eq) in DCM (1.00 mL) was added TFA (616 mg, 5.40 mmol, 400 μL, 75.4 eq) at 0° C. The reaction mixture was warmed to RT and stirred for 1 h. The reaction mixture was concentrated under reduced pressure to give a residue.

The residue was diluted with sat. aq. NaHCO₃ and extracted with DCM. The combined organic layers were dried over Na₂SO₄ and concentrated under reduced pressure to afford 5-((S)-2-amino-3,3-dicyclopropylpropanamido)-4-fluoro-2-(1-oxo-1-((2,2,2-trifluoroethyl)amino)propan-2-yl)benzamide trifluoroacetate (40 mg, crude. TFA salt) as a yellow solid. LCMS [M+H]⁺=459.3 m/z.

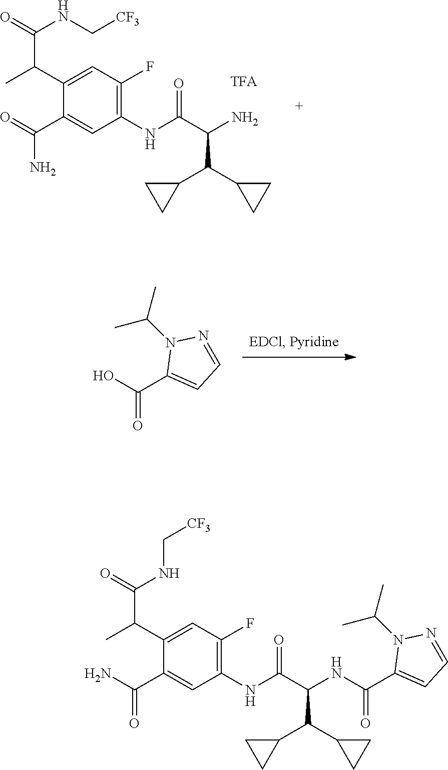 [SMILES: "TFA" ; CC(C(=O)NCC(F)(F)F)c1cc(F)c(NC(=O)[C@@H](N)C(C2CC2)C2CC2)cc1C(N)=O ; CC(C)n1nccc1C(=O)O ; "EDCl, Pyridine" ; CC(C(=O)NCC(F)(F)F)c1cc(F)c(NC(=O)[C@@H](NC(=O)c2ccnn2C(C)C)C(C2CC2)C2CC2)cc1C(N)=O]

N-((2S)-1-((5-carbamoyl-2-fluoro-4-(1-oxo-1-(2,2,2-tritluoroethyl)amino)propan-2-yl)phenyl)amino)-3,3-dicyclopropyl-1-oxopropan-2-yl)-1-isopropyl-1H-pyrazole-5-carboxamide (116). To a solution of 5-((S)-2-amino-3,3-dicyclopropylpropanamido)-4-fluoro-2-(1-oxo-1-((2,2,2-trifluoroethyl)amino)propan-2-yl)benzamide trifluoroacetate (36.0 mg, 78.5 umol, 1.00 eq) and 1-isopropyl-1H-pyrazole-5-carboxylic acid (14.5 mg, 94.2 umol, 1.20 eq) in pyridine (3.00 mL) was added EDCI (30.1 mg, 157 umol, 2.00 eq). The reaction mixture was stirred at RT for 12 h. The reaction mixture was concentrated under reduced pressure, diluted with sat. aq. NaHCO₃, extracted with DCM, washed with water and brine, dried over Na₂SO₄, and concentrated under reduced pressure to give a residue. The residue was purified by prep-TLC (10:1 DCM:MeOH) to afford N-((2S)-1-((5-carbamoyl-2-fluoro-4-(1-oxo-1-((2,2,2-trifluoroethyl)amino)propan-2-yl)phenyl)amino)-3,3-dicyclopropyl-1-oxopropan-2-yl)-1-isopropyl-1H-pyrazole-5-carboxamide (30.0 mg, 50.5 umol, 64.3% yield) as a yellow solid. The title compound was isolated as the first eluting, single stereoisomer by chiral SFC purification (column: DAICEL CHIRALPAK AD-H (250 mm×30 mm, 5 um), mobile phase: 20% 10.1% NH₃H₂O in IPA]). LCMS [M+H]⁺=595.4

### Example 123: Synthesis of Compound 117

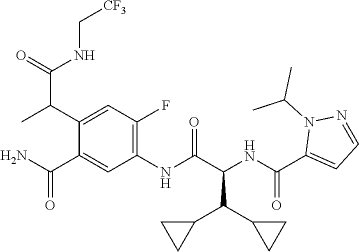 [SMILES: CC(C(=O)NCC(F)(F)F)c1cc(F)c(NC(=O)[C@@H](NC(=O)c2ccnn2C(C)C)C(C2CC2)C2CC2)cc1C(N)=O]

N-((2S)-1-((5-carbamoyl-2-fluoro-4-(1-oxo-1-((2,2,2-trifluoroethyl)amino)propan-2-yl)phenvl)amino)-3,3-dicyclopropyl-1-oxopropan-2-yl)-1-isopropyl-1H-pyrazole-5-carboxamide (117). The title compound was isolated as the second eluting, single stereoisomer by chiral SFC purification (column: DAICEL CHIRALPAK AD-H (250 mm×30 mm, 5 un): mobile phase: 20% 10.1% NH₃H₂O in IPA|). LCMS [M+H⁺=595.4.

### Example 124: Synthesis of Compound 118

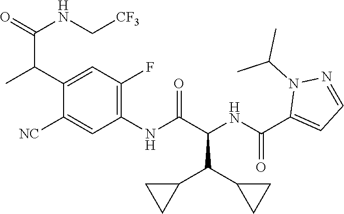 [SMILES: CC(C(=O)NCC(F)(F)F)c1cc(F)c(NC(=O)[C@@H](NC(=O)c2ccnn2C(C)C)C(C2CC2)C2CC2)cc1C#N]

N-((2S)-1-((5-cyano-2-fluoro-4-(1-oxo-1-((2,2,2-trifluoroethyl)amino)propan-2-yl)phenyl)amino)-3,3-dicyclopropyl-1-oxopropan-2-yl)-1-isopropyl-1H-pyrazole-5-carboxamide (118). Title compound was isolated as the second eluting, single stereoisomer by chiral SFC purification (column: REGIS (S,S) WHELK-Ol (250×25 mm, 10 um); mobile phase: 15% [0.1% NH₃H₂O in IPA]). LCMS [M+H]⁺=577.2 m/z.

### Example 125: Synthesis of Compound 119

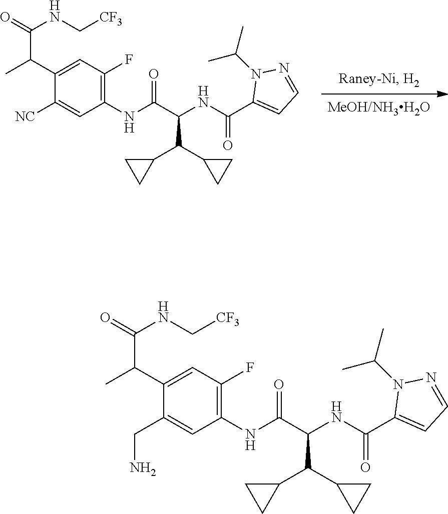 [SMILES: CC(C(=O)NCC(F)(F)F)c1cc(F)c(NC(=O)[C@@H](NC(=O)c2ccnn2C(C)C)C(C2CC2)C2CC2)cc1C#N ; "MeOH/NH3·H2O" ; "Raney-Ni, H2" ; CC(C(=O)NCC(F)(F)F)c1cc(F)c(NC(=O)[C@@H](NC(=O)c2ccnn2C(C)C)C(C2CC2)C2CC2)cc1CN]

N-((2S)-1-((5-(aminomethyl)-2-fluoro-4-(1-oxo-1-((2,2,2-trifluoroethyl)amino)propan-2-yl)phenyl)amino)-3,3-dicyclopropyl-1-oxopropan-2-yl)-1-isopropyl-1H-pyrazole-5-carboxamide (119). To a solution of N-((2S)-1-((5-cyano-2-fluoro-4-(1-oxo-1-((2,2,2-trifluoroethyl)amino)propan-2-yl)phenyl)amino)-3,3-dicyclopropyl-1-oxopropan-2-yl)-1-isopropyl-1H-pyrazole-5-carboxamide (20.0 mg, 34.7 umol, 1.00 eq) in MeOH (20.0 mL) and NH₃.H₂O (2.73 g, 19.5 mmol, 3.00 mL, 25% purity, 562 eq) was added Raney-Ni (5.00 mg, 58.4 umol, 1.68 eq) under N₂. The reaction mixture was degassed and purged with H₂ (3x) and stirred at RT for 5 h.

The reaction mixture was filtered and concentrated under reduced pressure to give a residue. The residue was purified by prep-HPLC (eluting with 40-70% ACN in water with 10 10 mM NH₄HCO₃) to afford N-((2S)-1-((5-(aminomethyl)-2-fluoro-4-(1-oxo-1-((2,2,2-trifluoroethyl)amino)propan-2-yl)phenyl)amino)-3,3-dicyclopropyl-1-oxopropan-2-yl)-1-isopropyl-1H-pyrazole-5-carboxamide (3.00 mg, 5.17 umol, 20.0% yield) as a yellow solid. LCMS [M+H]⁺=581.4 m/z.

### Example 126: Synthesis of Compound 120

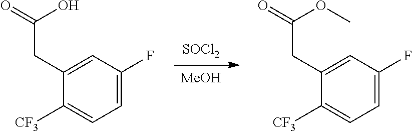

Methyl 2-(5-fluoro-2-(trifluoromethyl)phenyl)acetate. To a solution of 2-(5-fluoro-2-(trifluoromethyl)phenyl)acetic acid (3.00 g, 13.5 mmol, 1.00 eq) in MeOH (20.0 mL) was added SOCl₂ (3.21 g, 27.0 mmol, 1.96 mL, 2.00 eq), dropwise, at 0 C. The reaction mixture was then stirred for 2 h at RT. The reaction mixture was diluted with EtOAc and washed with sat. aq. NaHCO₃. The organic layers were dried over Na₂SO₄. filtered, and concentrated under reduced pressure to afford methyl 2-(5-fluoro-2-(trifluoromethyl)phenyl)acetate (3.00 g, 12.7 mmol, 94.1% yield) as yellow oil. ¹H NMR (400 MHz, CDCl₃): δ 7.68-7.65 (m, 1H), 7.15-7.07 (m, 2H), 3.83 (s, 2H), 3.73 (s, 3H).

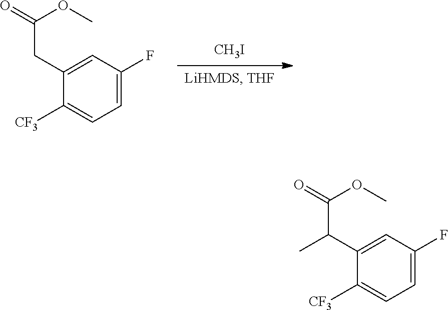

methyl 2-(5-fluoro-2-(trifluoromethyl)phenyl)propanoate. To a solution of methyl 2-(5-fluoro-2-(trifluoromethyl)phenyl)acetate (3.00 g, 12.7 mmol, 1.00 eq) in THF (10.0 mL) was added LiHMDS (I M, 19.0 mL, 1.50 eq), dropwise, at −10° C. and stirred for 0.5 h at −10° C. Then CH₃I (1.64 g, 11.6 mmol, 720 μL, 0.910 eq) was added dropwise to the reaction mixture and stirred for 1 h at 0° C. The reaction mixture was treated with sat. aq. NH₄Cl and extracted with EtOAc. The combined organic layers were washed with brine, dried over Na₂SO₄, filtered, and concentrated under reduced pressure to give a residue, which was purified by column chromatography (SiO₂, 0-30% EtOAc in hexanes) to afford methyl 2-(5-fluoro-2-(trifluoromethyl)phenyl)propanoate (2.00 g, 7.99 mmol, 62.9% yield) as yellow oil. ¹H NMR (400 MHz, CDCl₃). d 7.67-7.64 (m, 1H), 7.26-7.23 (m, 1H), 7.06 (t, J=2.4 Hz, l H), 4.19-4.13 (m, 1H), 3.69 (s, 3H), 1.51 (d, J=6.8 Hz, 3H).

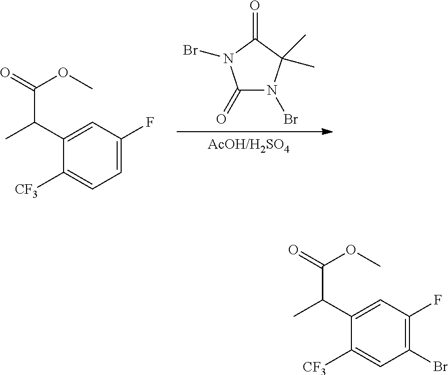

methyl 2-(4-bromo-5-fluoro-2-(trifluoromethyl)phenyl)propanoate. To a solution of methyl 245-fluoro-2-(trifluoromethyl)phenyl)propanoate (780 mg, 3.12 mmol, 1.00 eq) in H₂SO₄ (5.00 mL) and AcOH (1.00 mL) was added 1,3-dibromo-5,5-dimethylimidazolidine-2,4-dione (535 mg, 1.87 mmol, 0.600 eq) at 0° C. and then stirred for 4 h at 45° C. The reaction mixture was added to 100 mL of ice water, and the pH was adjusted to 8-9 with sat. aq. NaHCO₃. The solution was extracted with EtOAc, washed with brine, dried over Na₂SO₄, filtered, and concentrated under reduced pressure to give a residue, which was purified by column chromatography (SiO₂, 0-10% EtOAc in petroleum ether) followed by prep-HPLC (65-90% ACN in water buffered with formic acid) to afford methyl 2-(4-bromo-5-fluoro-2-(trifluoromethyl)phenyl)propanoate (900 mg, 2.73 mmol, 87.7% yield) as a yellow oil. ¹H NMR (400 MHz, CD₃CN): d 7.97 (d, J=6.8 Hz, 1H), 7.39 (d, J=9.6 Hz, 1H), 4.11-4.03 (m, 1H), 3.63 (s, 3H), 1.47 (d, J=7.2 Hz, 3H).

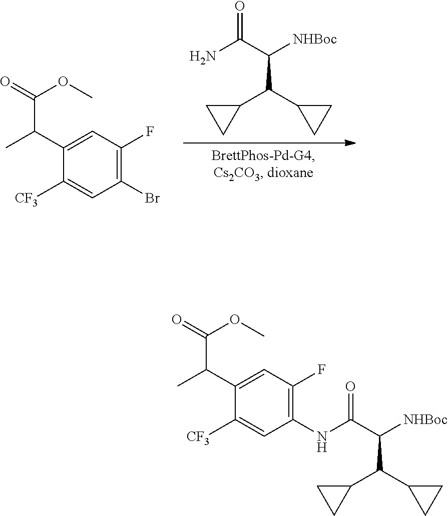 [SMILES: COC(=O)C(C)c1cc(F)c(Br)cc1C(F)(F)F ; CC(C)(C)OC(=O)N[C@H](C(N)=O)C(C1CC1)C1CC1 ; "BrettPhos-Pd-G4,Cs2CO3, dioxane" ; COC(=O)C(C)c1cc(F)c(NC(=O)[C@@H](NC(=O)OC(C)(C)C)C(C2CC2)C2CC2)cc1C(F)(F)F]

methyl 2-(4-((S)-2-((tert-butoxycarbonyl)amino)-3,3-dicyclopropylpropanamido)-5-fluoro-2-(trifluoromethyl)phenyl)propanoate. To a mixture of methyl 2-(4-bromo-5-fluoro-2-(trifluoromethyl)phenyl)propanoate (500 mg, 1.52 mmol, 1.00 eq), tert-butyl (S)-(1-amino-3,3-dicyclopropyl-1-oxopropan-2-yl)carbamate (489 mg, 1.82 mmol, 1.20 eq), and Cs₂CO₃ (1.49 g, 4.56 mmol, 3.00 eq) in dioxane (10.0 mL) was added BrettPhos-Pd-G4 (140 mg, 152 umol, 0.100 eq). The mixture was degassed and purged with N₂ 3× and stirred at 90° C. for 12 h under N₂. The reaction mixture was diluted with water and extracted with EtOAc. The combined organic layers were washed with brine, dried over Na₂SO₄, filtered, and concentrated under reduced pressure to give a residue, which was purified by prep-TLC (SiO₂, Petroleum ether:EtOAc=4:1) to afford methyl 2-(4-((S)-2-((tert-butoxycarbonyl)amino)-3.3-dicyclopropylpropanamido)-5-fluoro-2-(trifluoromethyl)phenyl)propanoate (350 mg, 678 umol, 44.6% yield) as yellow oil. ¹H NMR (400 MHz, CDCl₃): δ 8.72 (d, J=7.6 Hz, l H), 8.35 (d, J=4.8 Hz, 1H), 7.21-7.28 (m, 1H), 5.42 (s, 1H), 4.42 (s, 1H), 3.68 (d, J=1.6 Hz, 3H), 1.50-1.48 (m, 12H), 0.91-0.77 (m, 1H), 0.76-0.65 (m, 2H), 0.65-0.47 (m, 4H), 0.31-0.23 (m, 4H).

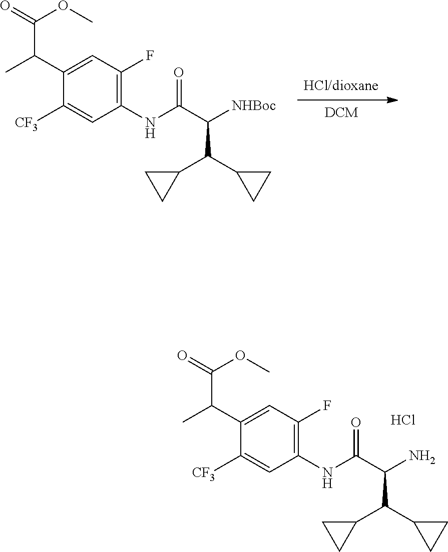

methyl 2-(4-((S)-2-amino-3,3-dicyclopropylpropanamido)-5-N₁uoro-2-(trifluoromethyl)phenyl)propanoate hydrochloride. To a mixture of methyl 2-(4-((S)-2-((tert-butoxycarbonyl)amino)-3,3-dicyclopropylpropanamido)-5-fluoro-2-(trifluoromethyl)phenyl)propanoate (350 mg, 678 umol, 1.00 eq) in DCM (1.50 mL) was added HCl/dioxane (4 M, 1.69 mL, 10.0 eq). The mixture was stirred at RT for 1 h, and the reaction mixture was concentrated under reduced pressure to afford methyl 2-(4-((S)-2-amino-3,3-dicyclopropylpropanamido)-5-fluoro-2-(trifluoromethyl)phenyl)propanoate hydrochloride (300 mg, crude, HCl) as a yellow oil. LCMS [M+H]⁺=417.2 m/^{z}.

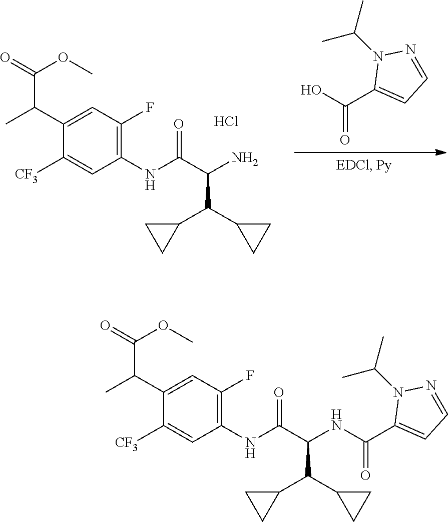

methyl 2-(4-((S)-3,3-dicyclopropyl-2-(1-isopropyl-1H-pyrazole-5-carboxamido)propanamido)-5-fluoro-2-(trifluoromethyl)phenyl)propanoate. To a solution of methyl 2-(4-((S)-2-amino-3,3-dicyclopropylpropanamido)-5-fluoro-2-(trifluoromethyl)phenyl)propanoate hydrochloride (300 mg, 662 umol, 1.00 eq. HCl) and 1-isopropyl-1H-pyrazole-5-carboxylic acid (123 mg, 795 umol, 1.20 eq) in pyridine (10.0 mL) was added EDCI (508 mg, 2.65 mmol, 4.00 eq). The reaction mixture was stirred for 1 h at RT. The reaction mixture was diluted with water and extracted with EtOAc. The combined organic layers were washed with brine, dried over Na₂SO₄. filtered, and concentrated under reduced pressure to give a residue, which was purified by prep-TLC (SiO₂. Petroleum ether:EtOAc=3:1) to afford methyl 2-(4-((S)-3,3-dicyclopropyl-2-(1-isopropyl-1H-pyrazole-5-carboxamido)propananido)-5-fluoro-2-(trifluoromethyl)phenyl)propanoate (330 mg, 597 umol, 90.2% yield) as a yellow solid.

LCMS [M+H]⁺=553.3 m/z.

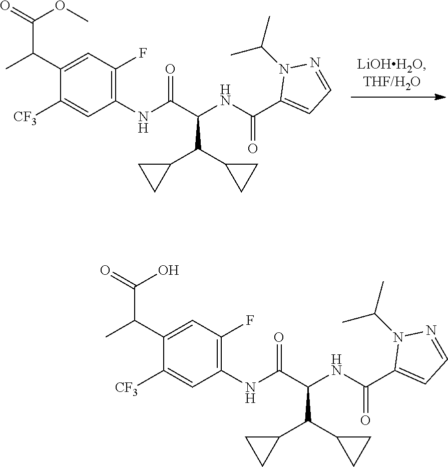 [SMILES: COC(=O)C(C)c1cc(F)c(NC(=O)[C@@H](NC(=O)c2ccnn2C(C)C)C(C2CC2)C2CC2)cc1C(F)(F)F ; "LiOH·H2O,THF/H2O" ; CC(C(=O)O)c1cc(F)c(NC(=O)[C@@H](NC(=O)c2ccnn2C(C)C)C(C2CC2)C2CC2)cc1C(F)(F)F]

2-(4-((S)-3,3-dicyclopropyl-2-(1-isopropyl-1H-pyrazole-5-carboxamido)propanamido)-5-fluoro-2-(trifluoromethyl)phenyl)propanoic acid. To a solution of methyl 2-(4-((S)-3,3-dicyclopropyl-2-(1-isopropyl-1H-pyrazole-5-carboxamido)propanamido)-5-fluoro-2-(trifluoromethyl)phenyl)propanoate (170 mg, 308 umol, 1.00 eq) in THF (3.00 mL) and H₂O (1.50 mL) was added LiOH·H₂O (25.8 mg, 615 umol, 2.00 eq) and stirred 12 h at RT. The reaction mixture was diluted with H₂O and adjusted pH to 3 with 1 M HCl and then extracted with EtOAc.

The combined organic layers were washed with brine, dried over Na₂SO₄. filtered, and concentrated under reduced pressure to give a residue, which was purified by prep-TLC (SiO₂, Petroleum ether:EtOAc=3:1) to afford 120 mg of a yellow solid. The product was further separated by chiral SFC purification (column: DAICEL CHIRALPAK IG (250 mm×30 mm, 10 um); mobile phase: 20% [0.1% NH1H₂O in MeOH]). LCMS [M+H]⁺=539.3 to afford the first eluting, single stereoisomer as peak 1 (45.0 mg, 83.6 umol, 27.2% yield, LCMS [M+H]⁺=539.3 m/z) as a white solid and the second eluting, single stereoisomer as peak 2 (40.0 mg, 74.3 umol, 24.1% yield, LCMS [M+H]⁺=539.3 m/:) as white solid.

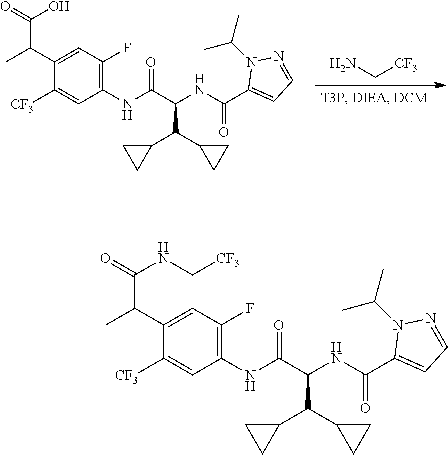

N-((2S)-1,1-dicyclopropyl-3-((2-fluoro-4-(1-oxo-1-((2,2,2-trifluoroethyl)amino)propan-2-yl)-5-(trifluoromethyl)phenyl)amino)-3-oxopropan-2-yl)-1-isopropyl-1 H-pyrazole-5-carboxamide (120). To a solution of peak 1 of 2-(4-((S)-3,3-dicyclopropyl-2-(1-isopropyl-1H-pyrazole-5-carboxamido)propanamido)-5-fluoro-2-(trifluoromethyl)phenyl)propanoic acid (45.0 mg, 83.6 umol, 1.00 eq), DIEA (54.0 mg, 418 umol, 72.8 uL, 5.00 eq), and 2,2,2-trifluoroethan-1-amine (9.93 mg, 100 umol, 7.88 uL, 1.20 eq) in DCM (2.00 mL) was added T3P (106 mg, 167 umol, 99.4 uL, 50.0% purity, 2.00 eq). The reaction mixture was stirred for 2 h at RT. The reaction mixture was diluted with water and extracted with EtOAc. The combined organic layers were washed with brine, dried over Na₂SO₄. filtered, and concentrated under reduced pressure to give a residue. The residue was purified by prep-TLC (SiO₂. Petroleum ether. EtOAc=3:1) to afford the first single stereoisomer N-((2S)-1,1-dicyclopropyl-3-((2-fluoro-4-(1-oxo-1-((2,2,2-trifluoroethyl)amino)propan-2-yl)-5-(trifluoromethyl)phenyl)amino)-3-oxopropan-2-yl)-1-isopropyl-1H-pyrazole-5-carboxamide (35.8 mg, 57.3 umol, 68.5% yield) as a white solid. LCMS [M+H]⁺=620.3 m/z.

### Example 127: Synthesis of Compound 121

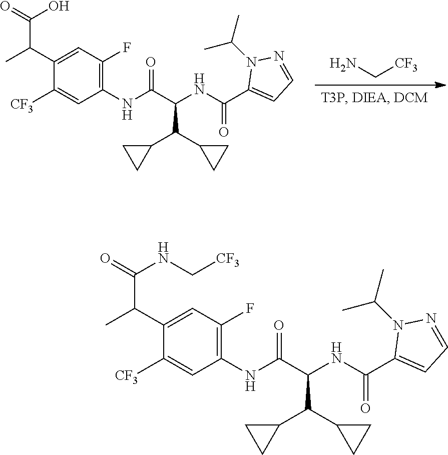

N-((2S)-1,1-dicyclopropyl-3-((2-fluoro-4-(1-oxo-1-((2,2,2-trifluoroethyl)amino)propan-2-yl)-5-(trifluoromethyl)phenyl)amino)-3-oxopropan-2-yl)-1-isopropyl-1H-pyrazole-5-carboxamide (121). To a solution of peak 2 of 2-(4-((S)-3,3-dicyclopropyl-2-(1-isopropyl-1H-pyrazole-5-carboxamido)propanamido)-5-fluoro-2(trifluoromethyl)phenyl)propanoic acid (40.0 mg, 74.3 umol, 1.00 eq), DIEA (48.0 mg, 371 umol, 64.7 uL, 5.00 eq), and 2,2,2-trifluoroethan-1-amine (8.83 mg, 89.1 umol, 7.01 uL, 1.20 eq) in DCM (2.00 mL) was added T3P (94.5 mg, 149 umol, 88.4 uL, 50.0% purity, 2.00 eq). The reaction mixture was stirred for 2 h at RT. The reaction mixture was diluted with water and extracted with EtOAc. The combined organic layers were washed with brine, dried over Na₂SO₄, filtered, and concentrated under reduced pressure to give a residue. The residue was purified by prep-TLC (SiO₂, Petroleum ether:EtOAc=3:1) to afford the second single stereoisomer of N-((2S)-1,1-dicyclopropyl-3-((2-fluoro-4-(1-oxo-1-((2,2,2-trifluoroethyl)amino)propan-2-yl)-5-(trifluoromethyl)phenyl)amino)-3-oxopropan-2-yl)-1-isopropyl-1H-pyrazole-5-carboxamide (25.3 mg, 40.6 umol, 54.7% yield) as a white solid. LCMS [M+H]⁺=620.3 m/z.

### Example 128: Synthesis of Compound 122

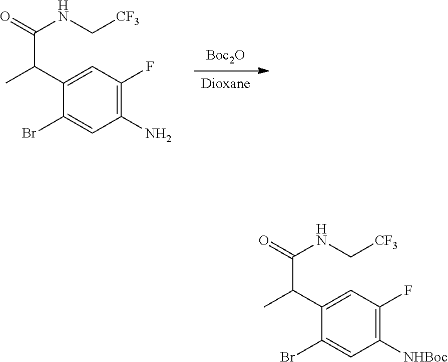 [SMILES: "Boc2O" ; CC(C(=O)NCC(F)(F)F)c1cc(F)c(N)cc1Br ; "Dioxane" ; CC(C(=O)NCC(F)(F)F)c1cc(F)c(NC(=O)OC(C)(C)C)cc1Br]

tert-butyl (5-bromo-2-fluoro-4-(1-oxo-1-((2,2,2-trifluoroethyl)amino)propan-2-yl)phenyl)carbamate. A solution of 2-(4-amino-2-bromo-5-fluorophenyl)-N-(2,2,2-trifluoroethyl)propanamide (0.10 g, 3.21 mmol, 1.00 eq) in Boc₂O (6.00 mL) was stirred at 100° C. for 15 h. The reaction mixture was concentrated under reduced pressure to give a residue. The residue was purified by column chromatography (SiO₂, Petroleum ether:EtOAc=30:1 to 3:1) to afford tert-butyl (5-bromo-2-fluoro-4-(1-oxo-1-((2,2,2-trifluoroethyl)amino)propan-2-yl)phenyl)carbamate (1.10 g, 2.48 mmol, 77.4% yield) as a yellow oil. LCMS [M+H-tBu]=387.0.

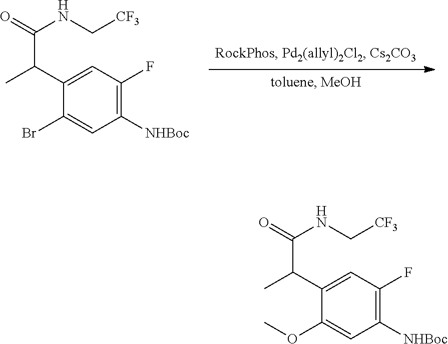 [SMILES: CC(C(=O)NCC(F)(F)F)c1cc(F)c(NC(=O)OC(C)(C)C)cc1Br ; "toluene, MeOH" ; COc1cc(NC(=O)OC(C)(C)C)c(F)cc1C(C)C(=O)NCC(F)(F)F]

tert-butyl (2-fluoro-5-methoxy-4-(1-oxo-1-((2,2,2-trifluoroethyl)amino)propan-2-yl)phenyl)carbamate. To a solution of tert-butyl (5-bromo-2-fluoro-4-(1-oxo-1-((2,2,2-trifluoroethyl)amino)propan-2-yl)phenyl)carbamate (300 mg, 677 umol, 1.00 eq) and MeOH (1.58 g, 49.4 mmol, 2.00 mL, 73.0 eq) in toluene (8.00 mL) was added RockPhos (31.7 mg, 67.7 umol, 0.100 eq), Cs2CO₃ (441 mg, 1.35 mmol, 2.00 eq) and Pd₂(allyl)₂C₁₂ (12.4 mg, 67.7 umol, 0.100 eq).

The mixture was stirred at 80° C. for 3 h. The mixture was filtered and the filtrate was concentrated to give a residue, which was purified by prep-TLC (SiO₂, Petroleum ether:EtOAc=3:1) to afford tert-butyl (2-fluoro-5-methoxy-4-(1-oxo-1-((2,2,2-trifluoroethyl)amino)propan-2-yl)phenyl)carbamate (140 mg, 355 umol, 52.5% yield) as yellow oil. LCMS [M+H-tBu]=339.0 m/z.

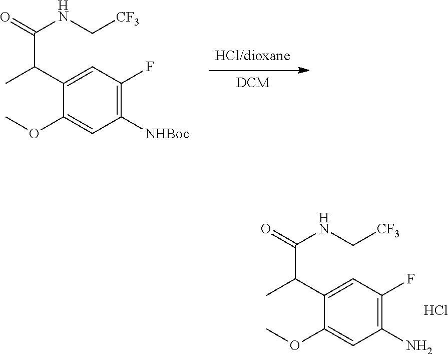

2-(4-amino-5-fluoro-2-methoxyphenyl)-N-(2,2,2-trifluoroethyl)propenamide hydrochloride. To a solution of tert-butyl (2-fluoro-5-methoxy-4-(1-oxo-1-((2,2,2-trifluoroethyl)amino)propan-2-yl)phenyl)carbamate (80.0 mg, 203 umol, 1.00 eq) in DCM (3.00 mL) was added HCl/dioxane (4.00 M, 5.00 mL, 98.6 eq) at 0° C., and the mixture was stirred at RT for 5.5 h. The reaction mixture was concentrated under reduced pressure to afford 2-(4-amino-5-fluoro-2-methoxyphenyl)-N-(2,2,2-trifluoroethyl)propenanide hydrochloride (70.0 mg. crude. HCl) as yellow solid. LCMS [M+H]⁺=295.0 m/z.

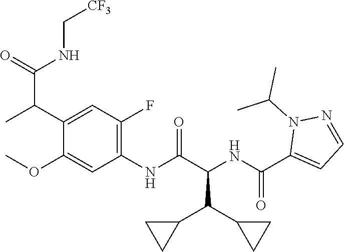 [SMILES: COc1cc(NC(=O)[C@@H](NC(=O)c2ccnn2C(C)C)C(C2CC2)C2CC2)c(F)cc1C(C)C(=O)NCC(F)(F)F]

N-((2S)-1,1-dicyclopropyl-3-((2-fluoro-5-methoxy-4-(1-oxo-1-((2,2,2-trifluoroethyl)amino)propan-2-yl)phenyl)amino)-3-oxopropan-2-yl)-1-isopropyl-1H-pyrazole-5-carboxamide (122). Prepared according to General Scheme D. employing 2-(4-amino-5-fluoro-2-methoxyphenyl)-N-(2,2,2-trifluoroethyl)propenamide hydrochloride and (S)-2-((tert-butoxycarbonyl)amino)-3,3-dicyclopropylpropanoic acid followed by 1-isopropyl-1H-pyrazole-5-carboxylic acid. The title compound was isolated as the first eluting, single stereoisomer by chiral SFC purification (column: DAICEL CHIRALPAK IG (250 mm×30 mm, 10 um); mobile phase: 20% 10.1% NNH₃H₂O in IPA|) as a white solid. LCMS [M+H]⁺=582.3 m/z.

### Example 129: Synthesis of Compound 123

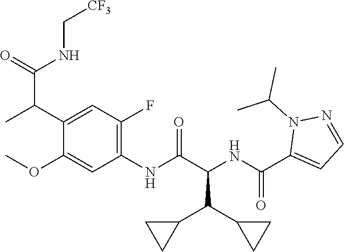 [SMILES: COc1cc(NC(=O)[C@@H](NC(=O)c2ccnn2C(C)C)C(C2CC2)C2CC2)c(F)cc1C(C)C(=O)NCC(F)(F)F]

N-((2S)-1,1-dicyclopropyl-3-((2-fluoro-5-methoxy-4-(1-oxo-1-((2,2,2-trifluoroethyl)amino)propan-2-yl)phenyl)amino)-3-oxopropan-2-yl)-1-isopropyl-1H-pyrazole-5-carboxamide (123). Prepared according to General Scheme D, employing 2-(4-amino-5-fluoro-2-methoxyphenyl)-N-(2,2,2-trifluoroethyl)propenamide hydrochloride and (S)-2-((tert-butoxycarbonyl)amino)-3,3-dicyclopropylpropanoic acid followed by 1-isopropyl-1H-pyrazole-5-carboxylic acid. The title compound was isolated as the second eluting, single stereoisomer by chiral SFC purification (column: DAICEL CHIRALPAK IG (250 mm×30 mm, 10 un), mobile phase: 20% [0.1% NH₃H₂O in IPA]) as a white solid. LCMS [M+H]⁺=582.3 m/z.

### Example 130: Synthesis of Compound 124

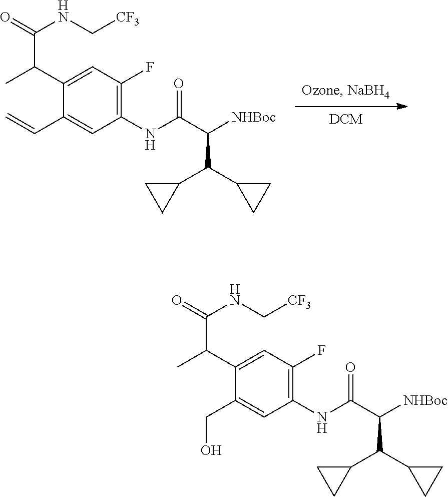

tert-butyl ((2S)-1,1-dicyclopropyl-3-((2-fluoro-5-(hydroxymethyl)-4-(1-oxo-1-((2,2,2-trifluoroethyl)amino)propan-2-yl)phenyl)amino)-3-oxopropan-2-yl)carbamate. OZONE (15 Psi) was bubbled into a solution of tert-butyl ((2S)-1,1-dicyclopropyl-3-((2-fluoro-4-(1-oxo-1-((2,2,2-trifluoroethyl)amino)propan-2-yl)-5-vinylphenyl)amino)-3-oxopropan-2-yl)carbamate (500 mg, 92.0 umol, 1.00 eq) in DCM (30.0 mL) and MeOH (3.0( ) mL) at −70° C. for 0.5 h followed by N₂ at −70° C. for 0.5)₂, then NaBH₄ (280 mg, 7.40 mmol, 8.02 eq) was added to the mixture and warmed to 20° C. and stirred for 2 h. The reaction mixture was treated with H₂O (3.0 mL) at 0° C. and then diluted with H₂O (60.0 mL) and extracted with DCM. The combined organic layers were dried over Na₂SO₄, filtered, and concentrated under reduced pressure to afford tert-butyl ((2S)-1,1-dicyclopropyl-3-((2-fluoro-5-(hydroyvmethyl)-4-(1-oxo-1-((2,2,2-trifluoroethyl)amino)propan-2-yl)phenyl)amino)-3-oxopropan-2-yl)carbamate (500 mg, 916 umol, 99.3% yield) as a white solid.

LCMS [M+H]⁺=546.4 m/z.

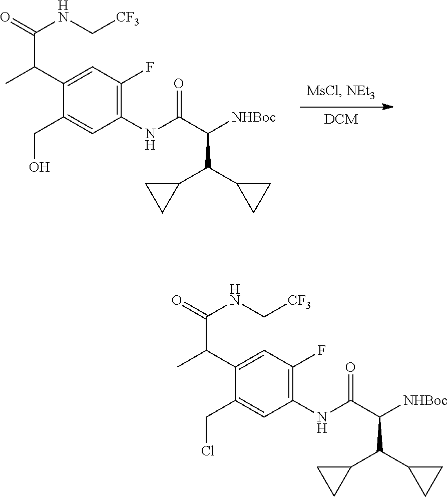

tert-butyl ((2S)-1-((5-(chloromethyl)-2-fluoro-4-(1-oxo-1-((2,2,2-trifluoroethyl)amino)propan-2-yl)phenyl)amino)-3,3-dicyclopropyl-1-oxopropan-2-yl)carbamate. To a solution of tert-butyl ((2S)-1,1-dicyclopropyl-3-((2-fluoro-5-(hydroyvmethyl)-4-(1-oxo-1-((2,2,2-trifluoroethyl)amino)propan-2-yl)phenyl)amino)-3-oxopropan-2-yl)carbamate (300 mg, 550 umol, 1.00 eq) and TEA (0.140 g, 1.38 mmol, 192 μL, 2.52 eq) in DCM (5.00 mL) was added MsCl (0.120 g, 1.05 mmol, 81.1 uL, 1.91 eq) at 0° C., then the mixture was stirred at RT for 1 h. The reaction mixture was treated with sat. aq. NaHCO₃ at 0° C. and extracted with DCM. The combined organic layers were dried over Na₂SO₄, filtered, and concentrated under reduced pressure to afford tert-butyl ((2S)-1-((5-(chloromethyl)-2-fluoro-4-(1-oxo-1-((2,2,2-trifluoroethyl)amino)propan-2-yl)phenyl)amino)-3,3-dicyclopropyl-1-oxopropan-2-yl)carbamate (300 mg, 531 umol, 96.7% yield) as a yellow solid. LCMS [M+H]⁺=564.2 m/z.

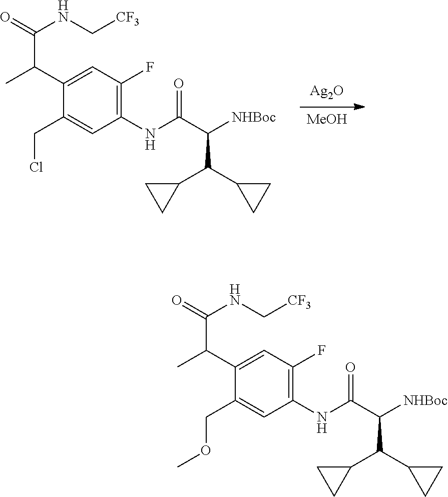 [SMILES: "MeOH" ; CC(C(=O)NCC(F)(F)F)c1cc(F)c(NC(=O)[C@@H](NC(=O)OC(C)(C)C)C(C2CC2)C2CC2)cc1CCl ; "Ag2O" ; COCc1cc(NC(=O)[C@@H](NC(=O)OC(C)(C)C)C(C2CC2)C2CC2)c(F)cc1C(C)C(=O)NCC(F)(F)F]

tert-butyl ((2S)-1,1-dicyclopropyl-3-((2-fluoro-5-(methoxymethyl)-4-(1-oxo-1-((2,2,2-trifluoroethyl)amino)propan-2-yl)phenyl)amino)-3-oxopropan-2-yl)carbamate. To a solution of tert-butyl ((2S)-1-((5-(chloromethyl)-2-fluoro-4-(1-oxo-1-((2,2,2-trifluoroethyl)amino)propan-2-yl)phenyl)amino)-3,3-dicyclopropyl-1-oxopropan-2-yl)carbamate (300 mg, 532 umol, 1.00 eq) in MeOH (10.0 mL) was added Ag₂O (246 mg, 1.06 mmol, 2.00 eq) and stirred at 50° C. for 12 h. The reaction mixture was filtered and concentrated under reduced pressure to give a residue. The residue was purified by prep-TLC (SiO₂, PE:EtOAc=2:1) to afford (2S)-2-amino-3,3-dicyclopropyl-N-(2-fluoro-5-(methoxymethyl)-4-(1-oxo-1-((2,2,2-trifluoroethyl)amino)propan-2-yl)phenyl)propanamide (0.160 g, 286 umol, 53.7% yield) as a white solid. LCMS [M+H]⁺=558.2 m/z. ¹H NMR (400 MHz, CDCl₃) δ 8.31-8.24 (m, 2H), 7.28-7.31 (m, 1H), 7.13 (d, J=12.0 Hz, 1H), 5.53-5.32 (m, 1H), 4.74 (d, J=10.8 Hz, 1H), 4.41 (s,1H), 4.23 (d, J=10.8 Hz, 1H), 3.94-3.82 (m, 1H), 3.80-3.76 (m, 1H), 3.60-3.48 (m, 1H), 3.46 (s, 3H), 1.48-1.43 (m, 12H), 0.90-0.75 (m, 3H), 0.58-0.51 (m, 4H), 0.29-0.23 (m, 4H).

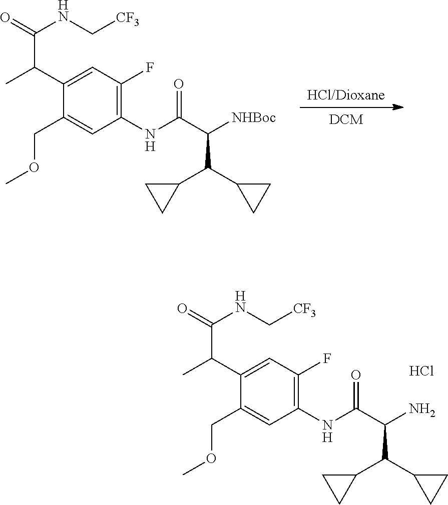

(2S)-2-amino-3,3-dicyclopropyl-N-(2-fluoro-5-(methoxymethyl)-4-(1-oxo-1-((2,2,2-trifluoroethyl)amino)propan-2-yl)phenyl)propenamide hydrochloride. To a solution of tert-butyl ((2S)-1,1-dicyclopropyl-3-((2-fluoro-5-(methoyvmethyl)-4-(1-oxo-1-((2,2,2-trifluoroethyl)amino)propan-2-yl)phenyl)amino)-3-oxopropan-2-yl)carbamate (130 mg, 232 umol, 1.00 eq) in DCM (3.00 mL) was added HCl/dioxane (4.00 M, 581 μL, 10.0 eq) and stirred at RT for 1 h. The reaction mixture was concentrated under reduced pressure to give afford (2S)-2-amino-3,3-dicyclopropyl-N-(2-fluoro-5-(methoxymethyl)-4-(1-oxo-1-((2,2,2-trifluoroethyl)amino)propan-2-yl)phenyl)propanamide (0.110 g, 222 umol, 95.5% yield, HCl) as a white solid. LCMS [M+H]⁺=460.2 m, z.

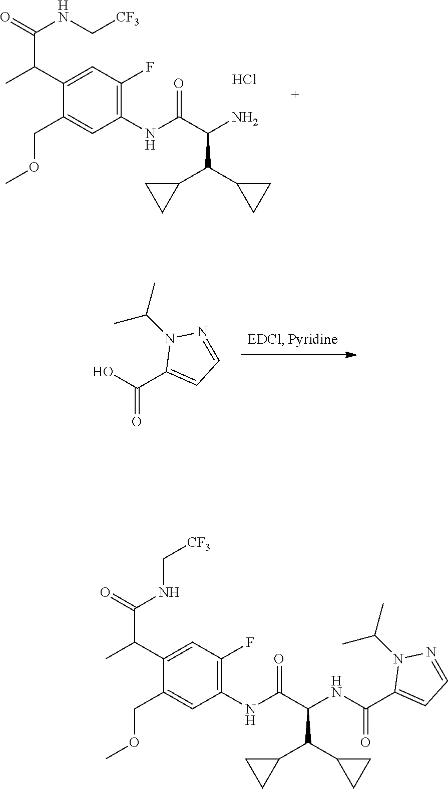 [SMILES: COCc1cc(NC(=O)[C@@H](N)C(C2CC2)C2CC2)c(F)cc1C(C)C(=O)NCC(F)(F)F ; Cl ; CC(C)n1nccc1C(=O)O ; "EDCl, Pyridine" ; COCc1cc(NC(=O)[C@@H](NC(=O)c2ccnn2C(C)C)C(C2CC2)C2CC2)c(F)cc1C(C)C(=O)NCC(F)(F)F]

N-((2S)-1,1-dicyclopropyl-3-((2-fluoro-5-(methoyvmethyl)-4-(1-oxo-1-((2,2,2-trifluoroethyl)amino)propan-2-yl)phenyl)amino)-3-oxopropan-2-yl)-1-isopropyl-1H-pyrazole-5-carboxamide. To a solution of compound (2S)-2-amino-3,3-dicyclopropyl-N-(2-fluoro-5-(methoxymethyl)-4-(1-oxo-1-((2,2,2-trifluoroethyl)amino)propan-2-yl)phenyl)propanamide (110 mg, 222 umol, 1.00 eq, HCl) and 1-isopropyl-1H-pyrazole-5-carboxylic acid (37.6 mg, 244 umol, 1.10 eq) in pyridine (3.00 mL) was added EDCI (127 mg, 665 umol, 3.00 eq) and stirred at RT for 6 h. The reaction mixture was diluted with H₂O and extracted with EtOAc. The combined organic layers were dried over Na₂SO₄, filtered, and concentrated under reduced pressure to give a residue. The residue was purified by prep-TLC (SiO₂, PE:EtOAc=1:1) to afford N-((2S)-1,1-dicyclopropyl-3-((2-fluoro-5-(methoxymethyl)-4-(1-oxo-1-((2,2,2-trifluoroethyl)amino)propan-2-yl)phenyl)amino)-3-oxopropan-2-yl)-1-isopropyl-1H-pyrazole-5-carboxamide (94.0 mg, 157 umol, 71.2% yield) as a white solid. LCMS [M+H]J=596.3 m/z.

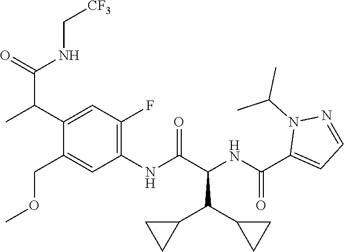 [SMILES: COCc1cc(NC(=O)[C@@H](NC(=O)c2ccnn2C(C)C)C(C2CC2)C2CC2)c(F)cc1C(C)C(=O)NCC(F)(F)F]

N-((2S)-1,1-dicyclopropyl-3-((2-fluoro-5-(methoxymethyl)-4-(1-oxo-1-((2,2,2-trifluoroethyl)amino)propan-2-yl)phenyl)amino)-3-oxopropan-2-yl)-1-isopropyl-1H-pyrazole-5-carboxamide. The title compound was isolated as the first eluting, single stereoisomer by chiral SFC purification (column: DAICEL CHIRALCEL OD (250 mm×30 mm, 10 um): mobile phase: 30% |0.1% NH₁-13H₂O in MEOH]). LCMS [M+H]⁺=596.3.

### Example 131: Synthesis of Compound 125

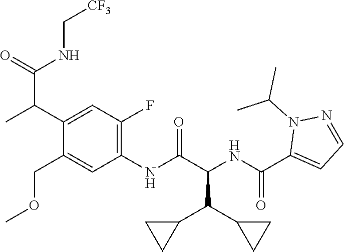 [SMILES: COCc1cc(NC(=O)[C@@H](NC(=O)c2ccnn2C(C)C)C(C2CC2)C2CC2)c(F)cc1C(C)C(=O)NCC(F)(F)F]

N-((2S)-1,1-dicyclopropyl-3-((2-fluoro-5-(methoxymethy )-4-(1-oxo-1-((2,2,2-trifluoroethyl)amino)propan-2-yl)phenyl)amino)-3-oxopropan-2-yl)-1-isopropyl-1H-pyrazole-5-carboxamide. The title compound was isolated as the second eluting of Example 130, single stereoisomer by chiral SFC purification (column: DAICEL CHIRALCEL OD (250 mm * 30 mm, 10 um); mobile phase: [0.1% NH₃H₂O MEOH]; B %: 20%-20%, 3.5 min; 20 minmin). LCMS [M+H]⁺=596.3.

### Example 132: Synthesis of Compound 126

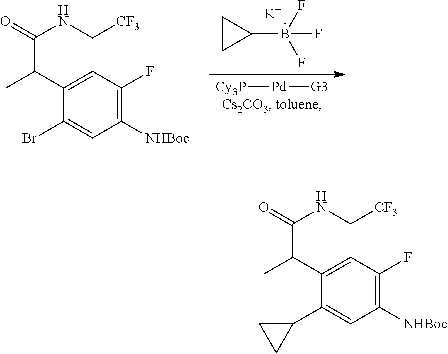

tert-butyl (5-cyclopropyl-2-fluoro-4-(1-oxo-1-((2,2,2-trifluoroethyl)amino)propan-2-yl)phenyl)carbamate. To a solution of tert-butyl (5-bromo-2-fluoro-4-(1-oxo-1-((2,2,2-trifluoroethyl)amino)propan-2-yl)phenyl)carbamate (50) mg, 1.13 mmol, 1.00 eq), potassium cyclopropyltrifluoroborate (200 mg, 1.35 mmol, 1.20 eq), and Cs2CO₃ (1.10 g, 3.38 mmol, 3.00 eq) in toluene (15.0 mL) was added Cy3P Pd-G& (73.3 mg, 113 umol, 0.100 eq), the mixture was stirred at 90° C. for 16 h. The reaction mixture was diluted with H₂O and extracted with EtOAc, dried over Na₂SO₄, filtered, and concentrated under reduced pressure to give a residue. The residue was purified by prep-TLC (PE:EtOAc=3:1) to afford tert-butyl (5-cyclopropyl-2-fluoro-4-(1-oxo-1-((2,2,2-trifluoroethyl)amino)propan-2-yl)phenyl)carbamate (160 mg, 396 umol, 35.0% yield) as a yellow solid. LCMS [M+H]⁺=405.3 m/z.

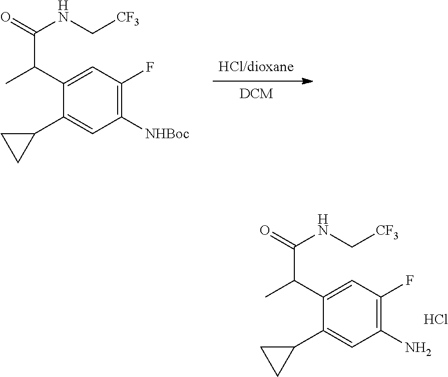

2-(4-amino-2-cyclopropyl-5-fluorophenyl)-N-(2,2,2-trifluoroethyl)propanamide hydrochloride. To a solution of tert-butyl (5-cyclopropyl-2-fluoro-4-(1-oxo-1-((2,2,2-trifluoroethyl) amino)propan-2-yl)phenyl)carbamate (160 mg, 396 umol, 1.00 eq) in DCM (2.00 mL) was added HCl/dioxane (4 M, 989 μL, 10.0 eq) at 0° C., and the mixture was stirred at RT for 30 mm. The reaction mixture was concentrated under reduced pressure to afford 2-(4-amino-2-cyclopropyl-5-fluorophenyl)-N-(2,2,2-trifluoroethyl)propanamide hydrochloride (130 mg, 382 umol, 96.4% yield. HCl) as white solid and was used directly in the next reaction.

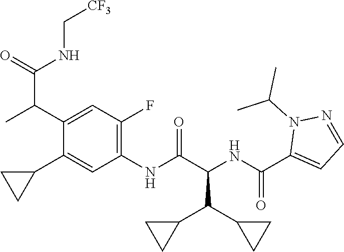 [SMILES: CC(C(=O)NCC(F)(F)F)c1cc(F)c(NC(=O)[C@@H](NC(=O)c2ccnn2C(C)C)C(C2CC2)C2CC2)cc1C1CC1]

N-((2S)-1,1-dicyclopropyl-3-((5-cyclopropyl-2-fluoro-4-(1-oxo-1-((2,2,2-trifluoroethyl)amino)propan-2-yl)phenyl)amino)-3-oxopropan-2-yl)-1-isopropyl-1H-pyrazole-5-carboxamide. Prepared according to General Scheme D. employing 2-(4-amino-2-cyclopropyl-5-fluorophenyl)-N-(2,2,2-trifluoroethyl)propanamide hydrochloride and (S)-2-((tert-butoxycarbonyl)amino)-3,3-dicyclopropylpropanoic acid followed by 1-isopropyl-1H-pyrazole-5-carboxylic acid. The title compound was isolated as the first eluting, single stereoisomer by chiral SFC purification (column: DAICEL CHIRALPAK IG (250 mm×30 mm, 10 um); mobile phase: 20% [0.1% NH₃H₂O in IPA]) as a white solid. LCMS [M+H]⁺=592.3 m/z.

### Example 133: Synthesis of Compound 127

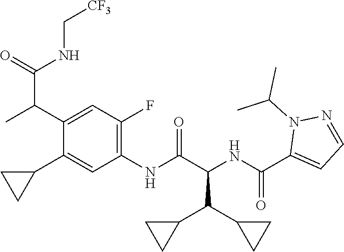 [SMILES: CC(C(=O)NCC(F)(F)F)c1cc(F)c(NC(=O)[C@@H](NC(=O)c2ccnn2C(C)C)C(C2CC2)C2CC2)cc1C1CC1]

N-((2S)-1,1-dicyclopropyl-3-((5-cyclopropyl-2-fluoro-4-(1-oxo-1-((2,2,2-trifluoroethyl)amino)propan-2-yl)phenyl)amino)-3-oxopropan-2-yl)-1-isopropyl-1H-pyrazole-5-carboxamide. Prepared according to General Scheme D (Example 132), employing 2-(4-amino-2-cyclopropyl-5-fluorophenyl)-N-(2,2,2-trifluoroethyl)propanamide hydrochloride and (S)-2-((tert-butoxycarbonyl)amino)-3,3-dicyclopropylpropanoic acid followed by 1-isopropyl-1H-pyrazole-5-carboxylic acid. The title compound was isolated as the second eluting of Example 132, single stereoisomer by chiral SFC purification (column: DAICEL CHIRALPAK IG (250 mm×30 mm, 10 um); mobile phase: 20% 10.1% NH₃H₂O in IPAJ) as a white solid. LCMS [M+H]⁺=592.3 m/^{z}.

### Example 134: Synthesis of Compound 128

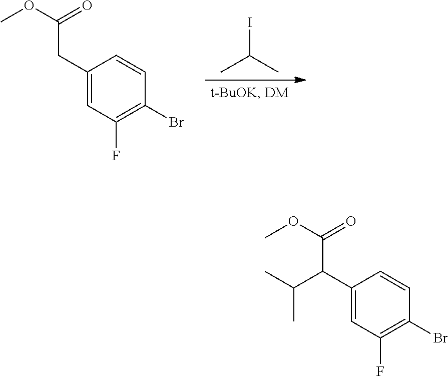

Methyl 2-(4-bromo-3-fluorophenyl)-3-methylbutanoate. To a solution of compound methyl 2-(4-bromo-3-fluorophenyl)acetate (1.50 g, 5.75 mmol, 1.00 eq) in DMF (15.0 mL) was added t-BuOK (709 mg, 6.32 mmol, 1.10 eq) was stirred at 0° C. for 0.5 h. Compound 2-iodopropane (976 mg, 5.75 mmol, 574 μL, 1.00 eq) was added to the above mixture at 0° C. for 0.5 h. The reaction mixture was diluted with 1 M HCl (80.0 mL), then extracted with EtOAc. The combined organic layers were washed with brine, dried over Na₂SO₄, filtered and concentrated under reduced pressure to give a residue. The residue was purified by column chromatography (SiO₂, Petroleum ether/EtOAc=30/1 to 1/1), TLC (plate 2, Petroleum ether: EtOAc=20: 1, Rf(Pl)=0.50).

Compound methyl 2-(4-bromo-3-fluorophenyl)-3-methylbutanoate (0.700 g, crude) was obtained as a yellow oil. LCMS [M+H]⁺=303.0 m/z.

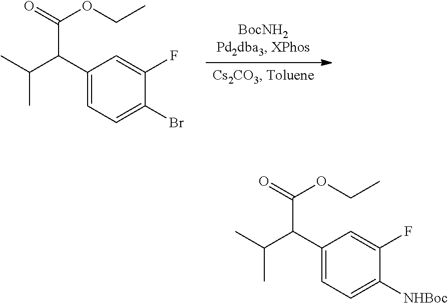 [SMILES: CCOC(=O)C(c1ccc(Br)c(F)c1)C(C)C ; "BocNH2Pd2dba3, XPhos" ; "Cs2CO3, Toluene" ; CCOC(=O)C(c1ccc(NC(=O)OC(C)(C)C)c(F)c1)C(C)C]

ethyl 2-(4-((tert-butoxycarbonyl)amino)-3-fluorophenyl)-3-methylbutanoate. To a solution of methyl 2-(4-bromo-3-fluorophenyl)-3-methylbutanoate (0.600 g, 1.98 mmol, 1.00 eq), BocNH₂ (278 mg, 2.37 mmol, 1.20 eq) in toluene (2.00 mL) was added Pd₂(dba)₃ (181 mg, 197 umol, 0.100 eq) and XPhos (94.3 mg, 197 umol, 0.100 eq), Cs₂CO. (1.29 g, 3.96 mmol, 2.00 eq). The mixture was stirred at 80° C. for 1 h. The reaction mixture was diluted with H₂O (30.0 mL), then extracted with EtOAc. The combined organic layers were washed with brine, dried over Na₂SO₄, filtered and concentrated under reduced pressure to give a residue. The residue was purified by column chromatography (SiO₂. Petroleum ether/EtOAc=10/1 to 0/1), TLC (plate 2, Petroleum ether: EtOAc=10: 1, Rf(P1)=0.40). Compound ethyl 2-(4-((tert-butoxycarbonyl)amino)-3-fluorophenyl)-3-methylbutanoate (0.400 g, crude) was obtained as a light yellow oil. LCMS [M+H]⁺=284.0 m/z.

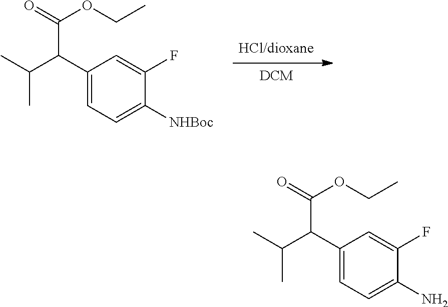

Ethyl 2-(4-amino-3-fluorophenyl)-3-methylbutanoate. To a solution of ethyl 2-(4-((tert-butoxycarbonyl)amino)-3-fluorophenyl)-3-methylbutanoate (0.400 g, 1.18 mmol, 1.00 eq) in DCM (2.00 mL) was added HCl/dioxane (4 M, 5.89 mL, 20.0 eq). The mixture was stirred at 0° C. for 1 h.

The reaction mixture was concentrated under reduced pressure to give a residue. Compound Ethyl 2-(4-amino-3-fluorophenyl)-3-methylbutanoate (0.500 g, crude. HCl) was obtained as a brown solid. LCMS [M+HJ* =240.0 m/z.

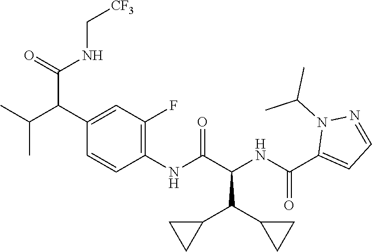 [SMILES: CC(C)C(C(=O)NCC(F)(F)F)c1ccc(NC(=O)[C@@H](NC(=O)c2ccnn2C(C)C)C(C2CC2)C2CC2)c(F)c1]

N-((2S)-1.1-dicyclopropyl-3-((2-fluoro-4-(3-methyl-1-oxo-1-((2,2,2-trifluoroethyl)amino)butan-2-yl)phenyl)amino)-3-oxopropan-2-yl)-1-isopropyl-1H-pyrazole-5-carboxamide. Prepared according to General Scheme F, employing Ethyl 2-(4-amino-3-fluorophenyl)-3-methylbutanoate and (S)-2-((tert-butoyvcarbonyl)amino)-3,3-dicyclopropylpropanoic acid followed by 2,2,2-trifluoroethan-1-amine hydrochloride and 1-isopropyl-1H-pyrazole-5-carboxylic acid. The title compound was isolated as the first eluting, single stereoisomer by chiral SFC purification (column: REGIS (R, R) WHELK-Ol (250 mm * 25 mm, 10 um); mobile phase: [0.1% NH₃H₂O MEOH]: B %: 35%-35%, 2.45: 30 min] as a white solid. LCMS [M+H]=580.3 m/z.

### Example 135: Synthesis of Compound 129

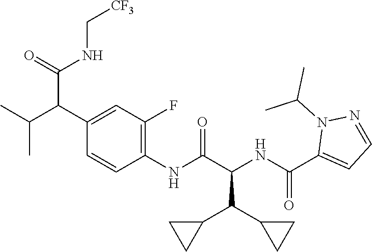 [SMILES: CC(C)C(C(=O)NCC(F)(F)F)c1ccc(NC(=O)[C@@H](NC(=O)c2ccnn2C(C)C)C(C2CC2)C2CC2)c(F)c1]

N-((2S)-1,1-dicyclopropyl-3-((2-fluoro-4-(3-methyl-1-oxo-1-((2,2,2-trifluoroethyl)amino)butan-2-yl)phenyl)amino)-3-oxopropan-2-yl)-1-isopropyl-1H-pyrazole-5-carboxamide. Prepared according to Example 134. The title compound was isolated as the second eluting, single stereoisomer by chiral SFC purification (column: REGIS (R, R) WHELK-Ol (250 mm×25 mm, 10 um); mobile phase: 35% [0.1% NH₃H₂O in MeOH]] as a white solid. LCMS [M+H]⁺=580.3 m/z.

### Example 136: Synthesis of Compound 130

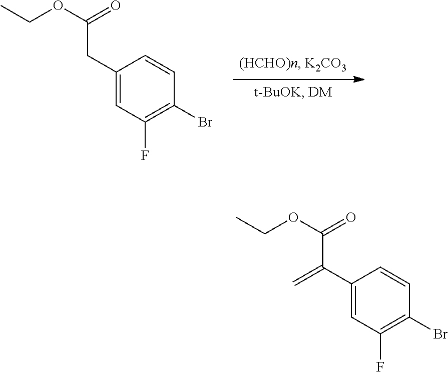

Ethyl 2-(4-bromo-3-fluorophenyl)acrylate. To a solution of ethyl 2-(4-bromo-3-fluorophenyl)acetate (2.00 g, 7.66 mmol, 1.00 eq), HCHO (460 mg, 15.3 mmol, 422 μL, 2.00 eq) in DMF (20.0 mL) was added K₂C₀₃ (2.54 g, 18.3 mmol, 2.40 eq) and TBAC (42.5 mg, 153 umol, 42.8 uL, 0.0200 eq). The mixture was stirred at 60° C. for 2 h. The reaction mixture was diluted with H₂O, then extracted with EtOAc. The combined organic layers were washed with brine, dried over Na₂SO₄, filtered, and concentrated under reduced pressure to afford ethyl 2-(4-bromo-3-fluorophenyl)acrylate (1.80 g, crude) as a yellow oil, which was used directly in the next reaction.

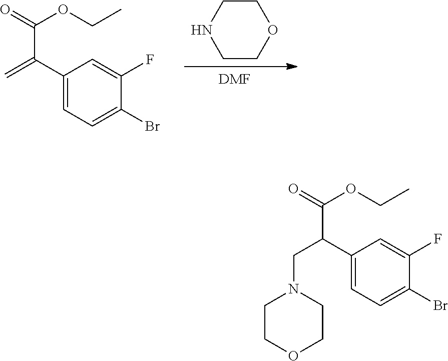

Ethyl 2-(4-bromo-3-fluorophenyl)-3-morpholinopropanoate. To a solution of methyl 2-(4-bromo-3-fluorophenyl)acrylate (1.80 g, 6.59 mmol, 1.00 eq) in DMF (2.00 mL) was added compound morpholine (2.87 g, 32.9 mmol, 2.90 mL, 5.00 eq). The mixture was stirred at 20° C. for 1 h. The reaction mixture was diluted with H₂O and extracted with EtOAc. The combined organic layers were washed with brine, dried over Na₂SO₄, filtered, and concentrated under reduced pressure to give a residue. The residue was purified by column chromatography (SiO₂, petroleum ether: EtOAc=10:1 to 5:1) to afford ethyl 2-(4-bromo-3-fluorophenyl)-3-morpholinopropanoate (0.350 g, crude) as a light yellow oil. LCMS [M+H]⁺=361.8 m/z.

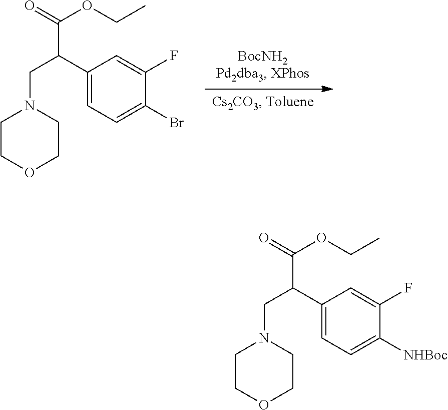 [SMILES: CCOC(=O)C(CN1CCOCC1)c1ccc(Br)c(F)c1 ; "BocNH2Pd2dba3, XPhos" ; "Cs2CO3, Toluene" ; CCOC(=O)C(CN1CCOCC1)c1ccc(NC(=O)OC(C)(C)C)c(F)c1]

Ethyl 2-(4-((tert-butoxycarbonyl)amino)-3-fluorophenyl)-3-morpholinopropanoate. To a solution of ethyl 2-(4-bromo-3-fluorophenyl)-3-morpholinopropanoate (300 mg, 832 umol, 1.00 eq) and BocNH₂(117 mg, 999 umol, 1.20 eq) in toluene (1.00 mL) was added Pd₂(dba)₃ (76.2 mg, 83.2 umol, 0.100 eq), XPhos (39.7 mg, 83.2 umol, 0.100 eq), and Cs₂CO₃ (542 mg, 1.67 mmol, 2.00 eq). The mixture was stirred at 80° C. for 2 h. The reaction mixture was cooled to RT, diluted with H₂O, and extracted with EtOAc. The combined organic layers were washed with brine, dried over Na₂SO₄, filtered, and concentrated under reduced pressure to give a residue. The residue was purified by Prep-TLC (SiO₂, petroleum ether: EtOAc=2:1) to afford ethyl 2-(4-((tert-butoxycarbonyl)amino)-3-fluorophenyl)-3-morpholinopropanoate (0.150 g, crude) as a red oil.

LCMS [M+H]=397.3 m/z.

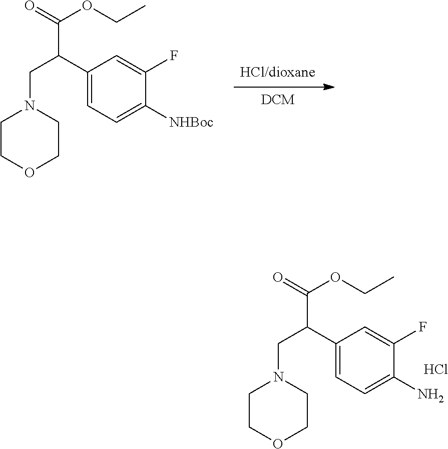

ethyl 2-(4-amino-3-fluorophenyl)-3-morpholinopropanoate. To a solution of ethyl 2-(4-((tert-butoxycarbonyl)amino)-3-fluorophenyl)-3-morpholinopropanoate (150 mg, 378 umol, 1.00 eq) in DCM (1.00 mL) was added HCl/dioxane (4.00 M, 1.89 mL, 20.0 eq). The mixture was stirred at 25° C. for 1 h. The reaction mixture was concentrated under reduced pressure to afford ethyl 2-(4-amino-3-fluorophenyl)-3-morpholinopropanoate (150 mg, crude, HCl) as a yellow solid.

LCMS [M+H]⁺=297.2 m/z.

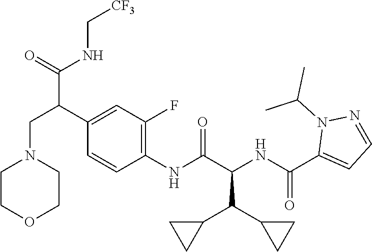 [SMILES: CC(C)n1nccc1C(=O)N[C@H](C(=O)Nc1ccc(C(CN2CCOCC2)C(=O)NCC(F)(F)F)cc1F)C(C1CC1)C1CC1]

N-((2S)-1,1-dicyclopropyl-3-((2-fluoro-4-(3-morpholino-1-oxo-1-((2,2,2-trifluoroethyl)amino)propan-2-yl)phenyl)amino)-3-oxopropan-2-yl)-1-isopropyl-1H-pyrazole-5-carboxamide. Prepared according to General Scheme F, employing ethyl 2-(4-amino-3-fluorophenyl)-3-morpholinopropanoate and (S)-2-((tert-butoxycarbonyl)amino)-3,3-dicyclopropylpropanoic acid followed by 2,2,2-trifluoroethan-1-amine hydrochloride and 1-isopropyl-1H-pyrazole-5-carboxylic acid. The title compound was isolated as the first eluting, single stereoisomer by chiral SFC purification (column: DAICEL CHIRALCEL OD (250 mm×30 mm, 10 um); mobile phase: 20% 10.1% NH₃H₂O MeOH]) as a white solid. LCMS [M+H]⁺=580.3 m/z.

### Example 137: Synthesis of Compound 131

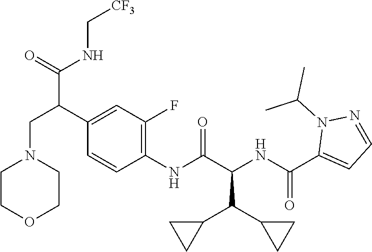 [SMILES: CC(C)n1nccc1C(=O)N[C@H](C(=O)Nc1ccc(C(CN2CCOCC2)C(=O)NCC(F)(F)F)cc1F)C(C1CC1)C1CC1]

N-((2S)-1,1-dicyclopropyl-3-((2-fluoro-4-(3-morpholino-1-oxo-1-((2,2,2-trifluoroethyl)amino)propan-2-yl)phenyl)amino)-3-oxopropan-2-yl)-1-isopropyl-1H-pyrazole-5-carboxamide. Prepared according to Example 136. The title compound was isolated as the second eluting, single stereoisomer by chiral SFC purification (column. DAICEL CHIRALCEL OD (250 mm×30 mm, 10 um): mobile phase: 20% 10.1% NH₃H₂O MeOH]) as a w % bite solid. LCMS [M+H]⁺=580.3 m/z.

### Example 138: Synthesis of Compound 132

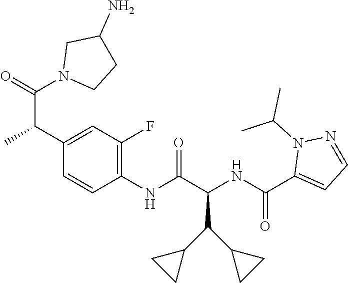 [SMILES: CC(C)n1nccc1C(=O)N[C@H](C(=O)Nc1ccc([C@H](C)C(=O)N2CCC(N)C2)cc1F)C(C1CC1)C1CC1]

N-((2S)-1-((4-((2S)-1-(3-aminopyrrolidin-1-yl)-1-oxopropan-2-yl)-2-fluorophenyl)amino)-3,3-dicyclopropyl-1-oxopropan-2-yl)-1-isopropyl-1H-pyrazole-5-carboxamide. Tert-butyl (1-((S)-2-(4-((S)-3,3-dicyclopropyl-2-(1-ethyl-1H-pyrazole-5-carboxamido)propanamido)-3-fluorophenyl)propanoyl)pyrrolidin-3-yl)carbamate was prepared according to General Scheme C, employing methyl (S)-2-(4-amino-3-fluorophenyl)propanoate and (S)-2-((tert-butoxycarbonyl)amino)-3,3-dicyclopropylpropanoic acid followed by 1-isopropyl-1H-pyrazole-5-carboxylic acid and tert-butyl pyrrolidin-3-ylcarbamate. The title compound was prepared according to General Procedure C. employing tert-butyl (1-((S)-2-(4-((S)-3,3-dicyclopropyl-2-(1-isopropyl-1H-pyrazole-5-carboxamido)propanamido)-3-fluorophenyl)propanoyl)pyrrolidin-3-yl)carbamate. LCMS [M+H]=539.4 m/z

### Example 139: Synthesis of Compound 133

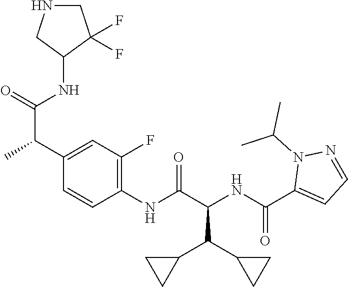 [SMILES: CC(C)n1nccc1C(=O)N[C@H](C(=O)Nc1ccc([C@H](C)C(=O)NC2CNCC2(F)F)cc1F)C(C1CC1)C1CC1]

N-((2S)-1,1-dicyclopropyl-3-((4-((2S)-1-((4,4-difluoropyrrolidin-3-yl)amino)-1-oxopropan-2-yl)-2-fluorophenyl)amino)-3-oxopropan-2-yl)-1-isopropyl-1H-pyrazole-5-carboxamide. Tert-butyl 4-((S)-2-(4-((S)-3,3-dicyclopropyl-2-(1-isopropyl-1H-pyrazole-5-carboxamido)propanamido)-3-fluorophenyl)propanamido)-3,3-difluoropyrrolidine-1-carboxylate was prepared according to General Scheme C, employing methyl (S)-2-(4-amino-3-fluorophenyl)propanoate and (S)-2-((tert-butoxycarbonyl)amino)-3,3-dicyclopropylpropanoic acid followed by 1-isopropyl-1H-pyrazole-5-carboxylic acid and tert-butyl 4-amino-3,3-difluoropyrrolidine-1-carboxylate. The title compound was prepared according to General Procedure C. employing tert-butyl 4-((S)-2-(4-((S)-3,3-dicyclopropyl-2-(1-isopropyl-1H-pyrazole-5-carboxamido)propanamido)-3-fluorophenyl)propanamido)-3,3-difluoropyrrolidine-1-carboxylate. The title compound was isolated as the first eluting, single stereoisomer by chiral SFC purification (column: REGIS (S, S) WHELK—01 (250 mm×25 mm, 10 um); mobile phase: 40% [0.1% NH₃H₂O MeOH]) as a white solid. LCMS [M+H]+=575.4 m/z.

### Example 140: Synthesis of Compound 134

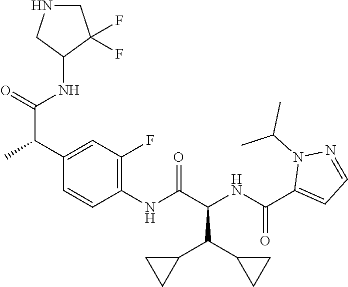 [SMILES: CC(C)n1nccc1C(=O)N[C@H](C(=O)Nc1ccc([C@H](C)C(=O)NC2CNCC2(F)F)cc1F)C(C1CC1)C1CC1]

N-((2S)-1,1-dicyclopropyl-3-((4-((2S)-1-((4,4-difluoropyrrolidin-3-yl)amino)-1-oxopropan-2-yl)-2-fluorophenyl)amino)-3-oxopropan-2-yl)-1-isopropyl-1H-pyrazole-5-carboxamide. Prepared according to Example 139. The title compound was isolated as the second eluting, single stereoisomer by chiral SFC purification (column: REGIS (S, S) WHELK—01 (250 mm×25 mm, 10 um); mobile phase: 40% [0.1% NH₃H₂O in MeOH]) as a white solid. LCMS [M+H]⁺=575.4 m/z.

### Example 141: Synthesis of Compound 135

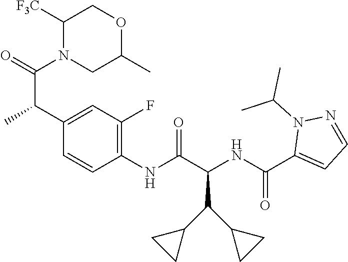 [SMILES: CC1CN(C(=O)[C@@H](C)c2ccc(NC(=O)[C@@H](NC(=O)c3ccnn3C(C)C)C(C3CC3)C3CC3)c(F)c2)C(C(F)(F)F)CO1]

N-((2S)-1,1-dicyclopropyl-3-((2-fluoro-4-((2S)-1-(2-methyl-5-(trifluoromethyl)morpholino)-1-oxopropan-2-yl)phenyl)amino)-3-oxopropan-2-yl)-1-isopropyl-1H-pyrazole-5-carboxamide. Prepared according to General Scheme C, employing methyl (S)-2-(4-amino-3-fluorophenyl)propanoate and (S)-2-((tert-butoxycarbonyl)amino)-3,3-dicyclopropylpropanoic acid followed by 1-isopropyl-1H-pyrazole-5-carboxylic acid and 2-methyl-5-(trifluoromethyl)morpholine. LCMS [M+H]⁺=622.3 m/z.

### Example 142: Synthesis of Compound 136

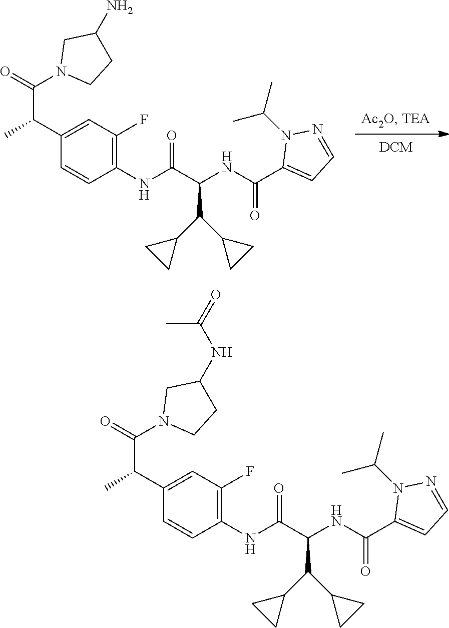

N-((2S)-1-((4-((2S)-1-(3-acetamidopyrrolidin-1-yl)-1-oxopropan-2-yl)-2-fluorophenyl)amino)-3,3-dicyclopropyl-1-oxopropan-2-yl)-1-isopropyl-1H-pyrazole-5-carboxamide. To a solution of N-((2S)-1-((4-((2S)-1-(3-aminopyrrolidin-1-yl)-1-oxopropan-2-yl)-2-fluorophenyl)amino)-3,3-dicyclopropyl-1-oxopropan-2-yl)-1-isopropyl-1H-pyrazole-5-carboxamide (25.0 mg, 43.3 umol, 1.00 eq. HCl) in DCM (1.00 mL) was added Ac₂O (8.85 mg, 86.6 umol, 8.12 uL, 2.00 eq) and TEA (13.2 mg, 130 umol, 18.1 uL, 3.00 eq) at 0° C. The mixture was stirred at 25° C. for 1 h. The residue was diluted with H₂O, extracted with EtOAc, dried over Na₂SO₄, filtered, and concentrated under reduced pressure to give a residue. The residue was purified by prep-HPLC (eluting with 33-63% ACN in water) to afford the title compound (4.39 mg, 7.51 umol, 17.3% yield) as a white solid. LCMS [M+H]⁺=581.4 m/z.

### Example 143: Synthesis of Compound 137

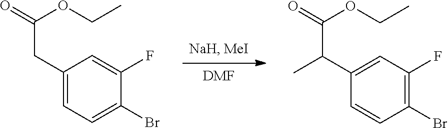

ethyl 2-(4-bromo-3-fluorophenyl)propanoate. To a solution of ethyl 2-(4-bromo-3-fluorophenyl)acetate (3.00 g, 11.5 mmol, 1.0) eq) in DMF (30.0 mL) was added NaH (597 mg, 14.9 mmol, 1.30 eq) at 0° C., the mixture was stirred at 0° C. for 0.5)₂, then CH₃I (1.40 g, 10.3 mmol, 644 μL, 0.900 eq) was added, the mixture was stirred at 25° C. for 1 h. The reaction mixture was quenched by sat.aq.NH₄Cl (30.0 mL), then extracted with EtOAc, the combined organic layer was washed with water, brine, dried over Na₂SO₄, filtered and concentrated under reduced pressure to give a residue. The residue was purified by column (SiO₂, petroleum ether: EtOAc=100: 0 to 20: 1, Plate 2, petroleum ether: EtOAc=3: 1, R_{f}=0.65) to afford ethyl 2-(4-bromo-3-fluorophenyl)propanoate (1.50 g, 5.4 mmol, 47.4% yield) as a yellow oil. ¹H NMR (400 MHz, DMSO-d₆) δ 7.66 (t, J=8.0 Hz, 1H), 7.33-7.30 (m, 1H), 7.11-7.08 (m, 1H), 4.09-4.03 (m, 2H), 3.87-3.81 (m, 1H), 1.37 (d, J=7.2 Hz, 3H).

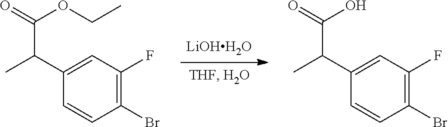

2-(4-bromo-3-fluorophenyl)propanoic acid. To a solution of ethyl 2-(4-bromo-3-fluorophenyl)propanoate (1.50 g, 5.45 mmol, 1.00 eq) in THF (15.0 mL) was added a solution of LiOH H₂O (457 mg, 10.9 mmol, 2.00 eq) in H₂O (3.00 mL) at 0° C., the mixture was stirred at 25° C. for 2 h. The reaction mixture was concentrated under reduced pressure, adjusted the pH=4 with 1 M HCl at 0° C., and extracted with EtOAc. The combined organic layer was washed with brine, dried over Na₂SO₄, filtered, and concentrated under reduced pressure afford 2-(4-bromo-3-fluorophenyl)propanoic acid (1.12 g, 4.53 mmol, 83.1% yield) as a yellow oil. ¹H NMR (400 MHz, DMSO-d₆) S 12.38 (brs, 1H), 7.65 (t, J=8.0 Hz, 1H), 7.33-7.28 (m, 1H), 7.11-7.08 (m, 1H), 3.76-3.71 (m, 1H), 1.36 (d, J=7.2 Hz, 3H).

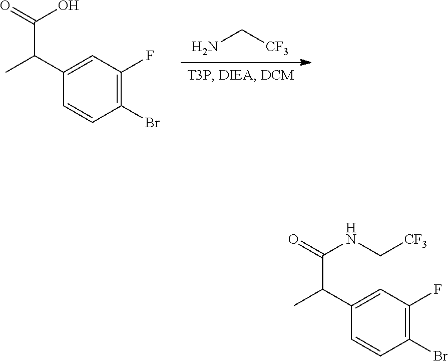

2-(4-bromo-3-fluorophenyl)-N-(2,2,2-trifluoroethyl)propanamide. To a solution of 2-(4-bromo-3-fluorophenyl)propanoic acid (900 mg, 3.64 mmol, 1.00 eq), 2,2,2-trifluoroethan-1-amine (397 mg, 4.01 mmol, 315 μL, 1.10 eq) in DCM (10.0 mL) was added DIPEA (2.35 g, 18.2 mmol, 3.17 mL, 5.00 eq) and T3P (4.64 g, 7.29 mmol, 4.33 mL, 2.00 eq). The mixture was stirred at 25° C. for 1 h. The reaction mixture was diluted with water and sat. aq. NaHCO₃ and extracted with DCM.

The combined organic layers were washed with brine, dried over Na₂SO₄, filtered, and concentrated under reduced pressure to afford 2-(4-bromo-3-fluorophenyl)-N-(2,2,2-trifluoroethyl)propanamide (1.00 g, 3.05 mmol, 83.6% yield) as a yellow oil. LCMS [M+H]⁺=329.9 m/z.

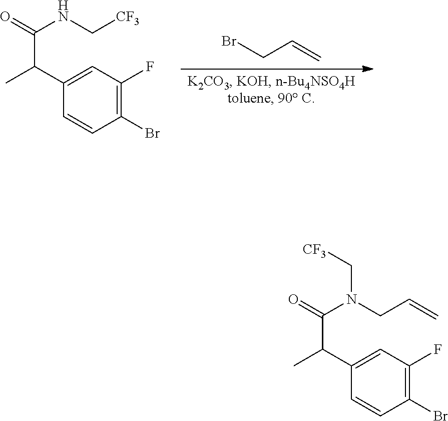

N-allyl-2-(4-bromo-3-fluorophenyl)-N-(2,2,2-trifluoroethyl)propanamide. To a solution of 2-(4-bromo-3-fluorophenyl)-N-(2,2,2-trifluoroethyl)propanamide (1.00 g, 3.05 mmol, 1.00 eq), 3-bromoprop-1-ene (369 mg, 3.05 mmol, 1.00 eq) in toluene (15.0 mL) was added K₂CO₃ (547 mg, 3.96 mmol, 1.30 eq), KOH (513 mg, 9.14 mmol, 3.00 eq), tetrabutylammonium hydrogen sulfate (103 mg, 305 umol, 0.100 eq), the mixture was stirred at 90° C. for 1 h. The reaction mixture was filtered and concentrated under reduced pressure, then diluted with EtOAc, washed with water, brine, dried over Na₂SO₄, filtered, and concentrated under reduced pressure to give a residue. The residue was purified by column (SiO₂, petroleum ether: EtOAc=100:1 to 10:1) to afford N-allyl-2-(4-bromo-3-fluorophenyl)-N-(2,2,2-trifluoroethyl)propanamide (800 mg, 2.17 mmol, 71.3% yield) as light yellow oil. LCMS [M+H]⁺=368.1 m/z.

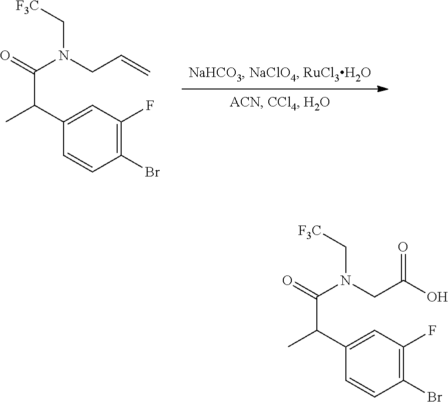

N-(2-(4-bromo-3-fluorophenyl)propanoyl)-N-(2,2,2-trifluoroethyl)glycine. To a solution of N-allyl-2-(4-bromo-3-fluorophenyl)-N-(2,2,2-trifluoroethyl)propanamide (500 mg, 1.36 mmol, 1.00 eq) in ACN (25.0 mL) was added CCl₄ (18.7 mL), H₂O (12.5 mL) and NaHCO₃ (570 mg, 6.79 mmol, 264 μL, 5.00 eq) followed by Na₁O₄ (2.32 g, 10.8 mmol, 602 μL, 8.00 eq) and RuCl₃·H₂O (91.8 mg, 407 umol, 0.300 eq) at 0° C. The mixture was stirred at RT for 2 h. The reaction mixture was filtered, and the filtrate was treated with sat. aq. NaHSO₃ at 0° C., then adjusted the pH=4 with 1 M HCl at 0° C. The solution was extracted with EtOAc, and the combined organic layers were washed with brine, dried over Na₂SO₄, filtered, and concentrated under reduced pressure to give a residue. The residue was purified by prep-TLC (SiO₂, petroleum ether:EtOAc=3:1) to afford N-(2-(4-bromo-3-fluorophenyl)propanoyl)-N-(2,2,2-trifluoroethyl)glycine (200 mg, 517 umol, 38.1% yield,) as a colorless oil. LCMS [M+H]⁺=386.0 m/z.

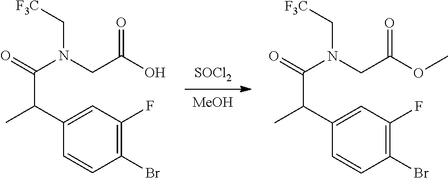

methyl N-(2-(4-bromo-3-fluorophenyl)propanoyl)-N-(2,2,2-trifluoroethyl)glycinate. To a solution of N-(2-(4-bromo-3-fluorophenyl)propanoyl)-N-(2,2,2-trifluoroethyl)glycine (200 mg, 517 umol, 1.00 eq) in MeOH (2.00 mL) was added SOCl₂ (123 mg, 1.04 mmol, 75.1 uL, 2.00 eq) at 0° C. The reaction mixture was stirred at RT for 2 h and concentrated under reduced pressure to afford methyl N-(2-(4-bromo-3-fluorophenyl)propanoyl)-N-(2,2,2-trifluoroethyl)glycinate (150 mg, crude) as a yellow oil. LCMS [M+H]⁺=400.1 m/z.

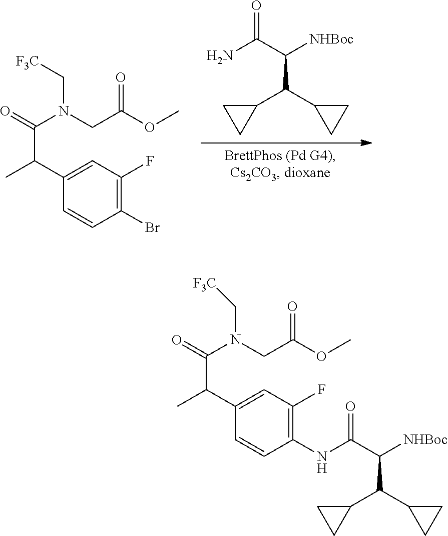

methyl N-(2-(4-((S)-2-((tert-butoxycarbonyl)amino)-3,3-dicyclopropylpropanamido)-3-fluorophenyl)propanoyl)-N-(2,2,2-trifluoroethyl)glycinate. To a solution of methyl N-(2-(4-bromo-3-fluorophenyl)propanoyl)-N-(2,2,2-trifluoroethyl)glycinate (100 mg, 250 umol, 1.00 eq) and tert-butyl (S)-(1-amino-3.3-dicyclopropyl-1-oxopropan-2-yl)carbamate (73.7 mg, 275 umol, 1.10 eq) in dioxane (5.00 mL) was added Cs2CO₃ (163 mg, 499 umol, 2.00 eq) and BrettPhos (Pd, G4) (23.0 mg, 25.0 umol, 0.100 eq). The mixture was stirred at 100° C. for 2 h under N₂. The reaction mixture was concentrated under reduced pressure to give a residue. The residue was purified by prep-TLC (SiO₂, petroleum ether: EtOAc=5:1) to afford methyl N-(2-(4-((S)-2-((tert-butoxycarbonyl)amino)-3,3-dicyclopropylpropanamido)-3-fluorophenyl)propanoyl)-N-(2,2,2-trifluoroethyl)glycinate (90.0 mg, 153 umol, 61.3% yield) as a yellow oil. LCMS [M+H]⁺=488.2 m/z. ¹H NMR (400 MHz, CDCl₃) δ 9.61 (s, 1H), 7.77-7.75 (m, 1H), 7.24-7.04 (m, 2H), 6.99 (d, J=6.4 Hz, 1H), 4.40-4.04 (m, 6H), 3.58 (d, J=16.0 Hz, 3H), 1.39 (s, 9H), 1.32-1.28 (m, 3H), 0.88-0.77 (m, 2H), 0.56-0.43 (m, 2H), 0.37-0.25 (m, 3H), 0.22-0.15 (m, 4H).

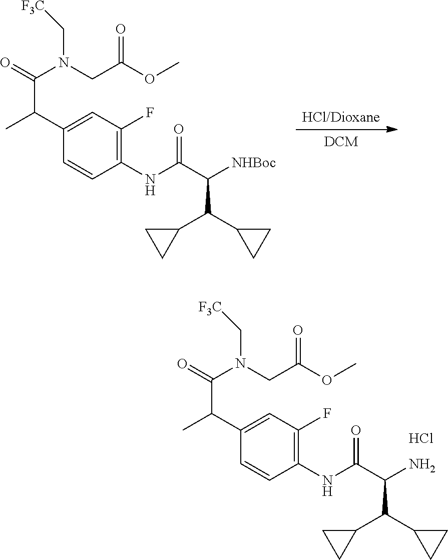

methyl N-(2-(4-((S)-2-amino-3,3-dicyclopropylpropanamido)-3-fluorophenyl)propanoyl)-N-(2,2,2-trifluoroethyl)glycinate. To a solution of methyl N-(2-(4-((S)-2-((tert-butoxycarbonyl)amino)-3,3-dicyclopropylpropanamido)-3-fluorophenyl)propanoyl)-N-(2,2,2-trifluoroethyl)glycinate (70.0 mg, 119 umol, 1.00 eq) in DCM (1.00 mL) was added HCl/dioxane (4 M, 1.00 mL, 33.5 eq) at 0° C., and the mixture was stirred at RT for 1 h. The reaction mixture was concentrated under reduced pressure to afford methyl N-(2-(4-((S)-2-amino-3,3-dicyclopropylpropanamido)-3-fluorophenyl)propanoyl)-N-(2,2,2-trifluoroethyl)glycinate (62.0 mg, 118 umol, 99.3% yield, HCl) as a yellow solid. LCMS [M+H]⁺=488.1 m/z.

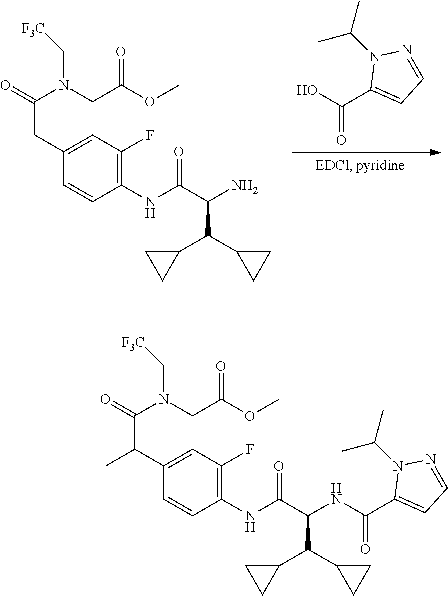

methyl N-(2-(4-((S)-3,3-dicyclopropyl-2-(1-isopropyl-1H-pyrazole-5-carboxamido)propanamido)-3-fluorophenyl)propanoyl)-N-(2,2,2-trifluoroethyl)glycinate. To a solution of methyl N-(2-(4-((S)-2-amino-3,3-dicyclopropylpropanamido)-3-fluorophenyl)propanoyl)-N-(2,2,2-trifluoroethyl)glycinate (62.0 mg, 118 umol, 1.00 eq, HCl), 1-isopropyl-1H-pyrazole-5-carboxylic acid (21.9 mg, 142 umol, 1.20 eq) in pyridine (1.00 mL) was added EDCI (45.3 mg, 237 umol, 2.00 eq), the mixture was stirred at 25° C. for 2 h. The reaction mixture was diluted with sat.aq.NaHCO₃, extracted with EtOAc, the combined organic layer was washed with brine, dried over Na₂SO₄, filtered and concentrated under reduced pressure to give a residue. The residue was purified by Prep-TLC (SiO₂, petroleum ether: EtOAc=1:1) to afford methyl N-(2-(4-((S)-3,3-dicyclopropyl-2-(1-isopropyl-1H-pyrazole-5-carboxamido)propanamido)-3-fluorophenyl)propanoyl)-N-(2,2,2-trifluoroethyl)glycinate (50.0 mg, 80.2 umol, 67.7% yield) as a white solid. LCMS [M+H]⁺=624.3 m/z.

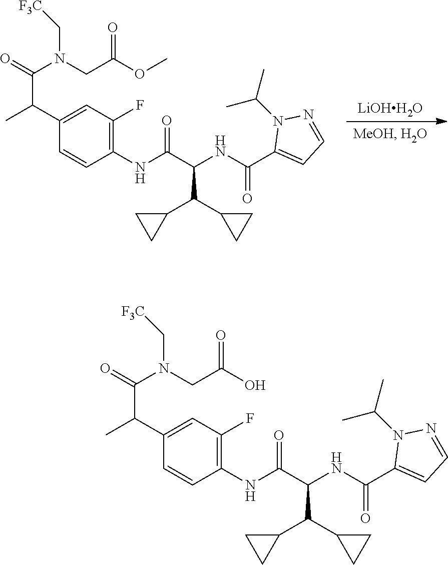

N-(2-(4-((S)-3,3-dicyclopropyl-2-(1-isopropyl-1H-pyrazole-5-carboxamido)propanamido)-3-fluorophenyl)propanoyl)-N-(2,2,2-trifluoroethyl)glycine. To a solution of methyl N-(2-(4-((S)-3,3-dicyclopropyl-2-(1-isopropyl-1H-pyrazole-5-carboxamido)propanamido)-3-fluorophenyl)propanoyl)-N-(2,2,2-trifluoroethyl)glycinate (50.0 mg, 80.1 umol, 1.00 eq) in MeOH (1.00 mL) was added a solution of LiOH H₂O (6.73 mg, 16( ) umol, 2.0) eq) in H₂O (0.200 mL) at 0 C. The mixture was stirred at RT for 1 h. The mixture was concentrated under reduced pressure and then diluted with water (5 mL), adjusted the pH to 4 with 1 M HCl at 0° C. then extracted with EtOAc. The combined organic layers were washed with brine, filtered, and concentrated under reduced pressure to afford N-(2-(4-((S)-3,3-dicyclopropyl-2-(1-isopropyl-1H-pyrazole-5-carboxamido)propanamido)-3-fluorophenyl)propanoyl)-N-(2,2,2-trifluoroethyl)glycine (45.0 mg, 73.8 umol, 92.0% yield) as a white solid. LCMS [M+H]⁺=610.4 m/z.

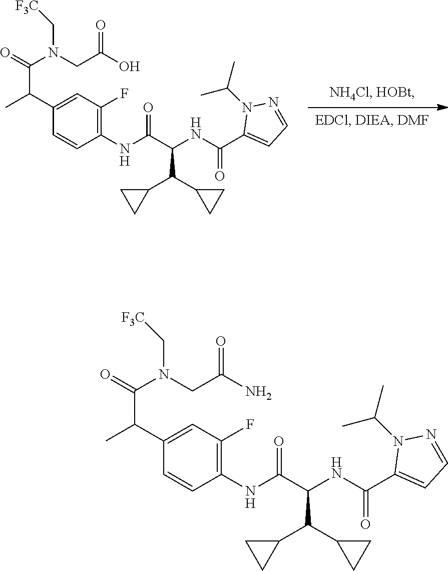 [SMILES: CC(C(=O)N(CC(=O)O)CC(F)(F)F)c1ccc(NC(=O)[C@@H](NC(=O)c2ccnn2C(C)C)C(C2CC2)C2CC2)c(F)c1 ; "EDCl, DIEA, DMF" ; "NH4Cl, HOBt," ; CC(C(=O)N(CC(N)=O)CC(F)(F)F)c1ccc(NC(=O)[C@@H](NC(=O)c2ccnn2C(C)C)C(C2CC2)C2CC2)c(F)c1]

N-((2S)-1-((4-(1-((2-amino-2-oxoethyl)(2,2,2-trifluoroethyl)amino)-1-oxopropan-2-yl)-2-fluorophenyl)amino)-3,3-dicyclopropyl-1-oxopropan-2-yl)-1-isopropyl-1H-pyrazole-5-carboxamide. To a solution of N-(2-(4-((S)-3,3-dicyclopropyl-2-(1-isopropyl-1 H-pyrazole-5-carboxamido)propanamido)-3-fluorophenyl)propanoyl)-N-(2,2,2-trifluoroethyl)glycine (45.0 mg, 73.8 umol, 1.00 eq) in DMF (1.00 mL) was added NH₄C₁ (19.7 mg, 369 uM, 1.50 eq), HOBt (19.9 mg, 147umol, 2.00 eq), DIPEA (28.6 mg, 221 umol, 38.5 uL, 3.00 eq), and EDCI (42.4 mg, 221 umol, 3.00 eq). The reaction mixture was stirred at RT for 2 h. The reaction mixture was diluted with water, extracted with EtOAc, washed with water, washed with brine, dried over sodium sulfate, filtered, and concentrated under reduced pressure to give a residue. The residue was purified by prep-TLC (EtOAc). The title compound was isolated as the first eluting, single stereoisomer by chiral SFC purification (column: DAICEL CHIRALPAK IC (250 mm×30 mm, 10 um), mobile phase: 35% [0.1% NH₃H₂O MEOH]) as a white solid. LCMS [M+H]⁺=609.3 m/z.

### Example 144: Synthesis of Compound 138

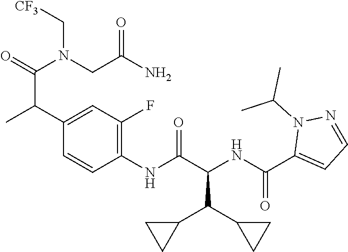 [SMILES: CC(C(=O)N(CC(N)=O)CC(F)(F)F)c1ccc(NC(=O)[C@@H](NC(=O)c2ccnn2C(C)C)C(C2CC2)C2CC2)c(F)c1]

N-((2S)-1-((4-(1-((2-amino-2-oxoethyl)(2,2,2-trifluoroethyl)amino)-1-oxopropan-2-yl)-2-fluorophenyl)amino)-3,3-dicyclopropyl-1-oxopropan-2-yl)-1-isopropyl-1H-pyrazole-5-carboxamide. Prepared according to Example 143. The title compound was isolated as the second eluting, single stereoisomer by chiral SFC purification (column: DAICEL CHIRALPAK IC (250 mm×30 mm, 10 um); mobile phase: 35% [0.1% NH₃H₂O in MEOH]) as a white solid. LCMS [M+H]⁺=609.3 m/z.

### Example 145: Synthesis of Compound 139

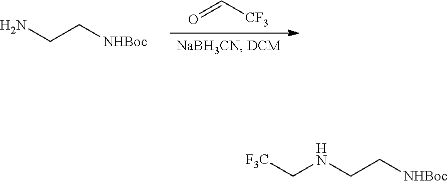

tert-butyl (2-((2,2,2-trifluoroethyl)amino)ethyl)carbamate. To a solution of tert-butyl (2-aminoethyl)carbamate (734 mg, 5.62 mmol, 3.00 eq) in DCM (2.00 mL) was added 2,2,2-trifluoroacetaldehyde (300 mg, 1.87 mmol, 294 μL, 1.00 eq). The resulting reaction mixture was stirred at 15° C. for 3 h. Then. NaBH₃CN (235 mg, 3.75 mmol, 2.00 eq) was added, and the reaction mixture was stirred at 15° C. for 1 h before filtering and concentrating under reduced pressure to give a residue. The residue was purified by prep-TLC (SiO₂, petroleum ether.EtOAc=1:1) to afford tert-butyl (2-((2,2,2-trifluoroethyl)amino)ethyl)carbamate (200 mg, 825 umol, 44.1% yield) as a colorless oil.

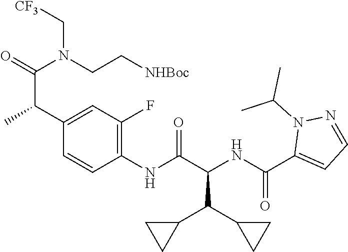 [SMILES: CC(C)n1nccc1C(=O)N[C@H](C(=O)Nc1ccc([C@H](C)C(=O)N(CCNC(=O)OC(C)(C)C)CC(F)(F)F)cc1F)C(C1CC1)C1CC1]

tert-butyl (2-((S)-2-(4-((S)-3,3-dicyclopropyl-2-(1-isopropyl-1H-pyrazole-5-carboxamido)propanamido)-3-fluorophenyl)-N-(2,2,2-trifluoroethyl)propanamido)ethyl)carbamate. Prepared according to General Scheme C. employing methyl (S)-2-(4-amino-3-fluorophenyl)propanoate and (S)-2-((tert-butoxycarbonyl)amino)-3,3-dicyclopropylpropanoic acid followed by 1-isopropyl-1H-pyrazole-5-carboxylic acid and tert-butyl (2-((2,2,2-trifluoroethyl)amino)ethyl)carbamate. LCMS [M+H]⁺=695.3 m/z.

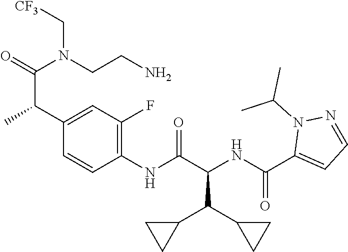 [SMILES: CC(C)n1nccc1C(=O)N[C@H](C(=O)Nc1ccc([C@H](C)C(=O)N(CCN)CC(F)(F)F)cc1F)C(C1CC1)C1CC1]

N-((S)-1-((4-((S)-1-((2-aminoethyl)(2,2,2-trifluoroethyl)amino)-1-oxopropan-2-yl)-2-fluorophenyl)amino)-3,3-dicyclopropyl-1-oxopropan-2-yl)-1-isopropyl-1H-pyrazole-5-carboxamide. Prepared using General Procedure A, employing tert-butyl (2-((S)-2-(4-((S)-3,3-dicyclopropyl-2-(1-isopropyl-1H-pyrazole-5-carboxamido)propananido)-3-fluorophenyl)-N-(2,2,2-trifluoroethyl)propanamido)ethyl)carbamate. LCMS [M+H]⁺=595.2 n:.

### Example 146: Synthesis of Compound 140

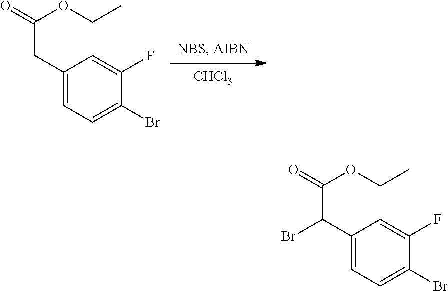

ethyl 2-bromo-2-(4-bromo-3-fluorophenyl)acetate. To a solution of ethyl 2-(4-bromo-3-fluorophenyl)acetate (1.50 g, 5.75 mmol, 1.00 eq) in CHCl₃ (15.0 mL) was added NBS (1.23 g, 6.89 mmol, 1.20 eq) and AIBN (94.3 mg, 574 umol, 0.100 eq). The mixture was stirred at 60° C. for 1 h. The reaction mixture was diluted with H₂O, then extracted with EtOAc. The combined organic layers were washed with brine, dried over Na₂SO₄, filtered, and concentrated under reduced pressure to afford ethyl 2-bromo-2-(4-bromo-3-fluorophenyl)acetate (2.20 g, crude) as a yellow oil.

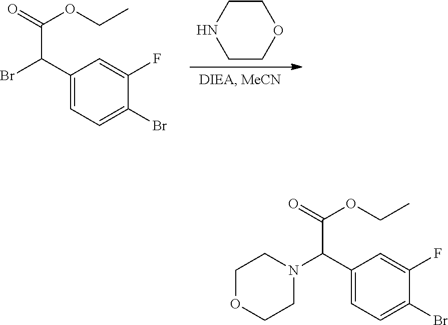

ethyl 2-(4-bromo-3-fluorophenyl)-2-morpholinoacetate. To a solution of ethyl 2-bromo-2-(4-bromo-3-fluorophenyl)acetate (2.00 g, 5.88 mmol, 1.00 eq) in ACN (10.0 mL) was added DIPEA (912 mg, 7.06 mmol, 1.23 mL, 1.20 eq) and morpholine (614 mg, 7.06 mmol, 621 μL, 1.20 eq). The mixture was stirred at RT for 1 h. The reaction mixture was diluted with H₂O, then extracted with EtOAc. The combined organic layers were washed with brine, dried over Na₂SO₄, filtered, and concentrated under reduced pressure to give a residue. The residue was purified by column chromatography (SiO₂, petroleum ether/EtOAc=15/1 to 10/1) to afford ethyl 2-(4-bromo-3-fluorophenyl)-2-morpholinoacetate (1.10 g, crude) as a light yellow oil. LCMS [M+H]—=347.9 m/z.

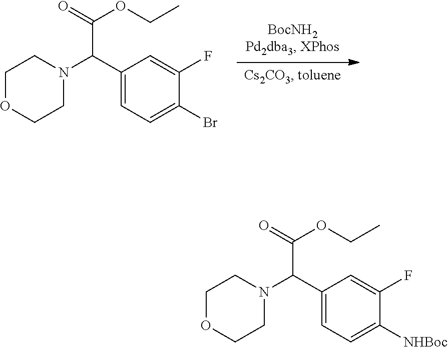 [SMILES: CCOC(=O)C(c1ccc(Br)c(F)c1)N1CCOCC1 ; "BocNH2Pd2dba3, XPhos" ; "Cs2CO3, toluene" ; CCOC(=O)C(c1ccc(NC(=O)OC(C)(C)C)c(F)c1)N1CCOCC1]

ethyl 2-(4-((tert-butoxycarbonyl)amino)-3-fluorophenyl)-2-morpholinoacetate. To a solution of ethyl 2-(4-bromo-3-fluorophenyl)-2-morpholinoacetate (50 mg, 1.44 mmol, 1.00 eq) and tert-butyl carbamate (203 mg, 1.73 mmol, 1.20 eq) in toluene (5.00 mL) was added Pd₂(dba)₃ (132 mg, 144 umol, 0.100 eq), XPhos (68.8 mg, 144 umol, 0.100 eq), and Cs2CO₃ (941 mg, 2.89 mmol, 2.00 eq). The mixture was stirred at 80° C. for 2 h. The reaction mixture was diluted with H₂O, then extracted with EtOAc. The combined organic layers were washed with brine, dried over Na₂SO₄, filtered, and concentrated under reduced pressure to give a residue. The residue was purified by prep-TLC (SiO₂, petroleum ether/EtOAc=3/1) to afford ethyl 2-(4-((tert-butoxycarbonyl)amino)-3-fluorophenyl)-2-morpholinoacetate (0.500 g, crude) as a red oil. LCMS [M+H]⁺=383.3 m/z.

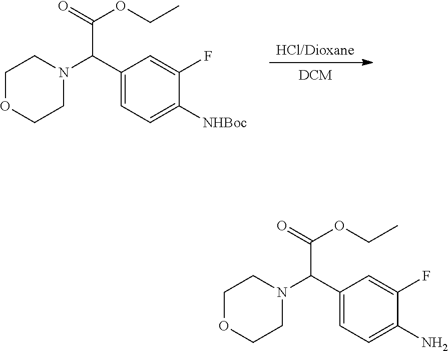

ethyl 2-(4-amino-3-fluorophenyl)-2-morpholinoacetate. To a solution of ethyl 2-(4-((tert- PGP422,C₃ butoxycarbonyl)amino)-3-fluorophenyl)-2-morpholinoacetate (0.5(O) g, 1.31 mmol, 1.00 eq) in DCM (5.00 mL) was added HCl/dioxane (4 M, 6.54 mL, 20.0 eq). The mixture was stirred at RT for 1 h. The reaction mixture was concentrated under reduced pressure to afford ethyl 2-(4-amino-3-fluorophenyl)-2-morpholinoacetate (0.400 g, crude, HCl) as a white solid. LCMS [M+H]⁺=283.1 m/z.

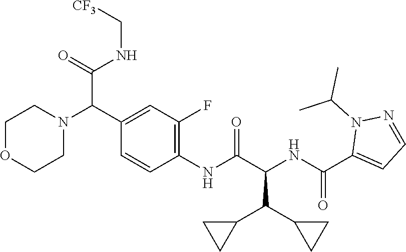 [SMILES: CC(C)n1nccc1C(=O)N[C@H](C(=O)Nc1ccc(C(C(=O)NCC(F)(F)F)N2CCOCC2)cc1F)C(C1CC1)C1CC1]

N-((2S)-1,1-dicyclopropyl-3-((2-fluoro-4-(1-morpholino-2-oxo-2-((2,2,2-trifluoroethyl)amino)ethyl)phenyl)amino)-3-oxopropan-2-yl)-1-isopropyl-1H-pyrazole-5-carboxamide. Prepared according to General Scheme C, employing ethyl 2-(4-amino-3-fluorophenyl)-2-morpholinoacetate and (S)-2-((tert-butoxycarbonyl)amino)-3,3-dicyclopropylpropanoic acid followed by 1-isopropyl-1H-pyrazole-5-carboxylic acid and 2,2,2-trifluoroethan-1-amine. The title compound was isolated as the first eluting, single stereoisomer by chiral SFC purification (column: REGIS(S, S) WHELK—Ol (250 mm×25 mm, 10 um); mobile phase: 55% [0.1% NH₃H₂O MeOH]) as a yellow solid. LCMS [M+H]—=623.3 m/z.

### Example 147: Synthesis of Compound 141

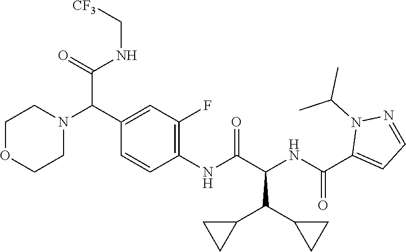 [SMILES: CC(C)n1nccc1C(=O)N[C@H](C(=O)Nc1ccc(C(C(=O)NCC(F)(F)F)N2CCOCC2)cc1F)C(C1CC1)C1CC1]

N-((2S)-1,1-dicyclopropyl-3-((2-fluoro-4-(1-morpholino-2-oxo-2-((2,2,2-trifluoroethyl)amino)ethyl)phenyl)amino)-3-oxopropan-2-yl)-1-isopropyl-1H-pyrazole-5-carboxamide. Prepared according to General Scheme C (Example 146), employing ethyl 2-(4-amino-3-fluorophenyl)-2-morpholinoacetate and (S)-2-((tert-butoxycarbonyl)amino)-3,3-dicyclopropylpropanoic acid followed by 1-isopropyl-1H-pyrazole-5-carboxylic acid and 2,2,2-trifluoroethan-1-amine. The title compound was isolated as the second eluting of Example 146, single stereoisomer by chiral SFC purification (column: REGIS(S, S) WHELK—O1 (250 mm×25 mm, 1Oum); mobile phase: 55% |0.1% NH₃H₂O MeOH|) as a yellow solid. LCMS [M+H₁⁹=623.3 m/z.

### Example 148: Synthesis of Compound 142

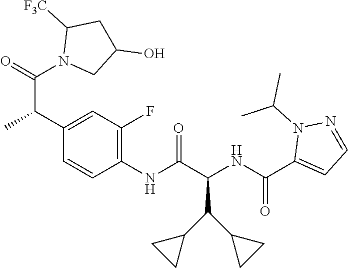 [SMILES: CC(C)n1nccc1C(=O)N[C@H](C(=O)Nc1ccc([C@H](C)C(=O)N2CC(O)CC2C(F)(F)F)cc1F)C(C1CC1)C1CC1]

N-((2S)-1,1-dicyclopropyl-3-((2-fluoro-4-((2S)-1-(4-hydroxy-2-(trifluoromethyl)pyrrolidin-1-yl)-1-oxopropan-2-yl)phenyl)amino)-3-oxopropan-2-yl)-1-isopropyl-1H-pyrazole-5-carboxamide. Prepared according to General Scheme C, employing methyl (S)-2-(4-amino-3-fluorophenyl)propanoate and (S)-2-((tert-butoxycarbonyl)amino)-3,3-dicyclopropylpropanoic acid followed by 1-isopropyl-1H-pyrazole-5-carboxylic acid and 5-(trifluoromethyl)pyrrolidin-3-ol. The title compound was isolated as the first eluting, single stereoisomer by chiral SFC purification (column: DAICEL CHIRALPAK AD-H (250 mm×30 mm, 5 um): mobile phase: 25% [0.1% NH₃H₂O in IPA]) as a white solid. LCMS [M+H]⁺=608.4 m/z.

### Example 149: Synthesis of Compound 143

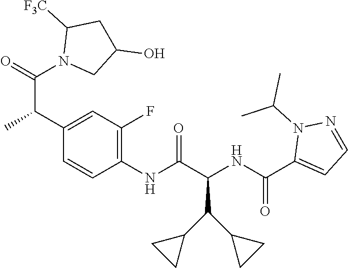 [SMILES: CC(C)n1nccc1C(=O)N[C@H](C(=O)Nc1ccc([C@H](C)C(=O)N2CC(O)CC2C(F)(F)F)cc1F)C(C1CC1)C1CC1]

N-((2S)-1,1-dicyclopropyl-3-((2-fluoro-4-((2S)-1-(4-hydroxy-2-(trifluoromethyl)pyrrolidin-1-yl)-1-oxopropan-2-yl)phenyl)amino)-3-oxopropan-2-yl)-1-isopropyl-1H-pyrazole-5-carboxamide. Prepared according to Example 148. The title compound was isolated as the second eluting, single stereoisomer by chiral SFC purification (column: DAICEL CHIRALPAK AD-H (250 mm×30 mm, 5 um): mobile phase: 25% [0.1% NH₃H₂O in IPA]) as a white solid. LCMS [M+H]⁺=608.4 m/z.

### Example 150: Synthesis of Compound 144

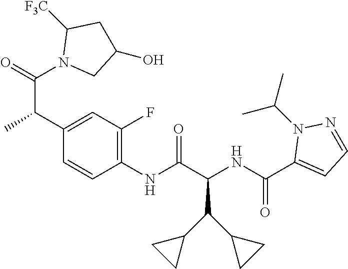 [SMILES: CC(C)n1nccc1C(=O)N[C@H](C(=O)Nc1ccc([C@H](C)C(=O)N2CC(O)CC2C(F)(F)F)cc1F)C(C1CC1)C1CC1]

N-((2S)-1,1-dicyclopropyl-3-((2-fluoro-4-((2S)-1-(4-hydroxy-2-(trifluoromethyl)pyrrolidin-1-yl)-1-oxopropan-2-yl)phenyl)amino)-3-oxopropan-2-yl)-1-isopropyl-1H-pyrazole-5-carboxamide. Prepared according to Example 148. The title compound was isolated as the third eluting, single stereoisomer by chiral SFC purification (column: DAICEL CHIRALPAK AD-H (250 mm×30 mm, 5 um): mobile phase: 25% [0.1% NH₃H₂O in IPA]) as a white solid. LCMS [M+H]⁺=608.3 m/z.

### Example 151: Synthesis of Compound 145

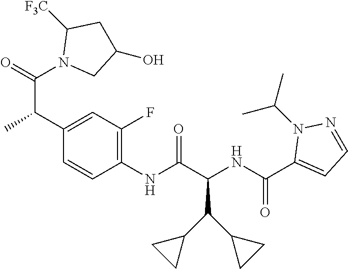 [SMILES: CC(C)n1nccc1C(=O)N[C@H](C(=O)Nc1ccc([C@H](C)C(=O)N2CC(O)CC2C(F)(F)F)cc1F)C(C1CC1)C1CC1]

N-((2S)-1,1-dicyclopropyl-3-((2-fluoro-4-((2S)-1-(4-hydroxy-2-(trifluoromethyl)pyrrolidin-1-yl)-1-oxopropan-2-yl)phenyl)amino)-3-oxopropan-2-yl)-1-isopropyl-1H-pyrazole-5-carboxamide. Prepared according to Example 148. The title compound was isolated as the fourth eluting, single stereoisomer by chiral SFC purification (column: DAICEL CHIRALPAK AD-H (250 mm×30 mm, 5 um); mobile phase: 25% [0.1% NH₃H₂O in IPA]) as a white solid. LCMS [M+H]⁺=608.3 m/z.

### Example 152: Synthesis of Compound 146

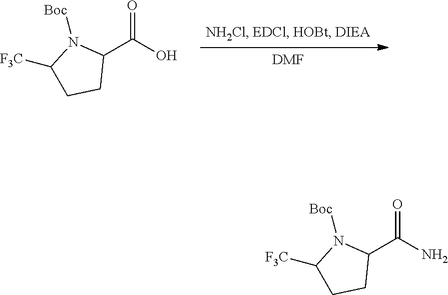

tert-butyl 2-carbamoyl-5-(trifluoromethyl)pyrrolidine-1-carboxylate. A mixture of 1-(tert-butoxycarbonyl)-5-(trifluoromethyl)pyrrolidine-2-carboxylic acid (0.300 g, 1.06 mmol, 1.00 eq), NH₄Cl (56.6 mg, 1.06 mmol, 1.00 eq), EDCI (406 mg, 2.12 mmol, 2.00 eq), HOBt (286 mg, 2.12 mmol, 2.00 eq), and DIPEA (342 mg, 2.65 mmol, 461 μL, 2.50 eq) in DMF (5.00 mL) was degassed and purged with N₂ (3x), and then the reaction mixture was stirred at RT for 12 h under a N₂ atmosphere. The reaction mixture was diluted with H₂O and extracted with EtOAc. The combined organic layers were dried over Na₂SO₄, filtered, and concentrated under reduced pressure to give a residue. The residue was purified by prep-TLC (SiO₂, PE:EtOAc=1:2) to afford tert-butyl 2-carbamoyl-5-(trifluoromethyl)pyrrolidine-1-carboxylate (0.130 g, 460 umol, 43.4% yield) as a white solid. ¹H NMR (400 MHz, DMSO-d₆) δ 7.02 (d, J=16.4 Hz, 2H), 4.49-4.43 (m, 1H), 4.11-4.06 (m, 1H), 2.25-1.82 (m, 4H), 1.38 (s, 9H).

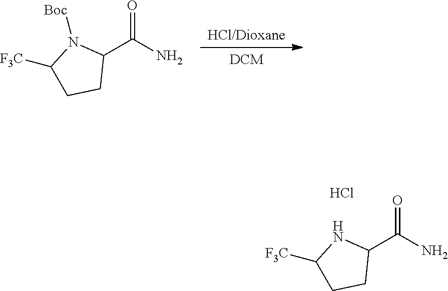

5-(trifluoromethyl)pyrrolidine-2-carboxamide. To a solution of tert-butyl 2-carbamoyl-5-(trifluoromethyl)pyrrolidine-1-carboxylate (0.130 g, 460 umol, 1.00 eq) in DCM (2.00 mL) was added HCU/dioxane (4 M, 1.15 mL, 10.0 eq) and stirred at RT for 1 h. The reaction mixture concentrated under reduced pressure to afford 5-(trifluoromethyl)pyrrolidine-2-carboxamide (0.100 g, 457 umol, 99.3% yield, HCl) as a yellow solid. LCMS [M+H]⁺=183.2 m/z.

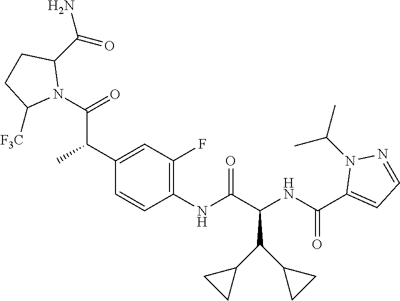 [SMILES: CC(C)n1nccc1C(=O)N[C@H](C(=O)Nc1ccc([C@H](C)C(=O)N2C(C(N)=O)CCC2C(F)(F)F)cc1F)C(C1CC1)C1CC1]

N-((2S)-1-((4-((2S)-1-(2-carbamoyl-5-(trifluoromethyl)pyrrolidin-1-yl)-¹-oxopropan-2-yl)-2-fluorophenyl)amino)-3,3-dicyclopropyl-1-oxopropan-2-yl)-1-isopropyl-1H-pyrazole-5-carboxamide. Prepared according to General Scheme C, employing methyl (S)-2-(4-amino-3-fluorophenyl)propanoate and (S)-2-((tert-butoxycarbonyl)amino)-3,3-dicyclopropylpropanoic acid followed by 1-isopropyl-1H-pyrazole-5-carboxylic acid and 5-(trifluoromethyl) pyrrolidine-2-carboxamide. The title compound was isolated as the first eluting stereoisomer by chiral SFC purification (column: REGIS(S, S) WHELK-Ol (250 mm×25 mm, 10 um); mobile phase: 40% NH₃H₂O in IPA]) as a white solid. LCMS [M+H]⁺=635.3 m/z.

### Example 153: Synthesis of Compound 147

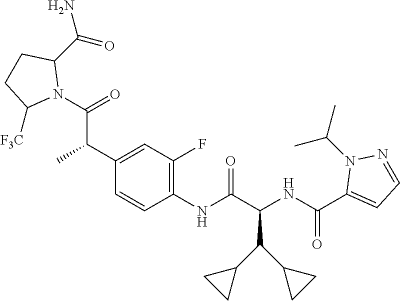 [SMILES: CC(C)n1nccc1C(=O)N[C@H](C(=O)Nc1ccc([C@H](C)C(=O)N2C(C(N)=O)CCC2C(F)(F)F)cc1F)C(C1CC1)C1CC1]

N-((2S)-1-((4-((2S)-1-(2-carbamoyl-5-(trifluoromethyl)pyrrolidin-1-yl)-1-oxopropan-2-yl)-2-fluorophenyl)amino)-3,3-dicyclopropyl-1-oxopropan-2-yl)-1-isopropyl-1H-pyrazole-5-carboxamide. Prepared according to Example 152. The title compound was isolated as the second eluting stereoisomer by chiral SFC purification (column: REGIS(S, S) WHELK-Ol (250 mm×25 mm, 10 um); mobile phase: 40% 10.1% NH₃H₂O in IPA]) as a white solid. LCMS [M+H]⁺=635.3 m/z.

### Example 154: Synthesis of Compound 148

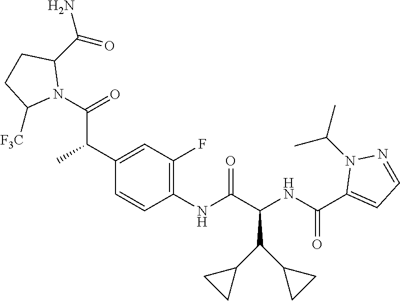 [SMILES: CC(C)n1nccc1C(=O)N[C@H](C(=O)Nc1ccc([C@H](C)C(=O)N2C(C(N)=O)CCC2C(F)(F)F)cc1F)C(C1CC1)C1CC1]

N-((2S)-1-((4-((2S)-1-(2-carbamoyl-5-(trifluoromethyl)pyrrolidin-1-yl)-1-oxopropan-2-yl)-2-fluorophenyl)amino)-3,3-dicyclopropyl-1-oxopropan-2-yl)-1-isopropyl-1H-pyrazole-5-carboxamide. Prepared according to Example 152. The title compound was isolated as the third eluting stereoisomer by chiral SFC purification (column: REGIS(S, S) WHELK-Ol (250 mm×25 mm, 10 um); mobile phase: 40% [0.1% NH₃H₂O in IPA]) as a white solid. LCMS [M+H]⁺=635.3 m/z.

### Example 155: Synthesis of Compound 149

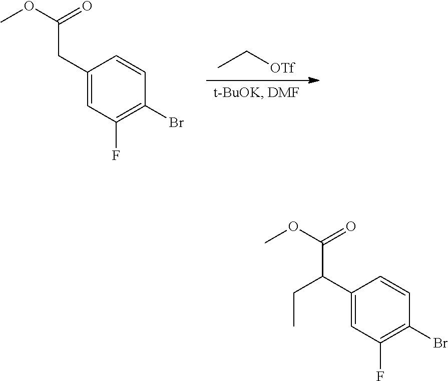

methyl 2-(4-bromo-3-fluorophenyl)butanoate. Methyl 2-(4-bromo-3-fluorophenyl)acetate (3.00 g, 12.1 mmol) was dissolved in DMF (12.0 mL), and the reaction solution was cooled to 0° C. A solution of potassium tert-butoxide (1.64 g, 14.6 mmol) in DMF (18.0 mL) was added dropwise, and the resulting reaction mixture was stirred for 30 min at 0° C. Ethyl trifluoromethanesulfonate (2.07 mL, 15.8 mmol) was then added at 0° C., and the reaction mixture was stirred 1 h at RT. The reaction mixture was diluted with water and was extracted with DCM. The organic solution was dried over MgSO₄, filtered, and concentrated under reduced pressure. The crude residue was purified by silica gel chromatography (0-20% EtOAc in hexanes) to afford methyl 2-(4-bromo-3-fluorophenyl)butanoate as a colorless oil (2.6 g, 79% yield). LCMS [M+H]⁺=275.0. ¹H NMR (CDCl₃, 400 MHz): δ 7.48 (t, J=7.7 Hz, 1H), 7.10 (dd, J=9.6, 2.0 Hz, 1H), 6.97 (dd, J=8.3, 1.9 Hz, 1H), 3.67 (s, 3H), 3.42 (t, .=7.7 Hz, 1H), 2.12-2.01 (m, 1H), 1.82-1.71 (m, 1H), 0.89 (t, J=7.4 Hz, 3H).

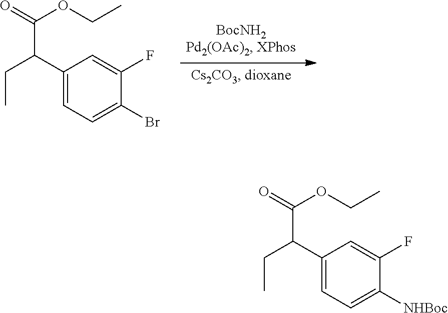 [SMILES: CCOC(=O)C(CC)c1ccc(Br)c(F)c1 ; "BocNH2Pd2(OAc)2, XPhos" ; "Cs2CO3, dioxane" ; CCOC(=O)C(CC)c1ccc(NC(=O)OC(C)(C)C)c(F)c1]

ethyl 2-(4-((tert-butoxycarbonyl)amino)-3-fluorophenyl)butanoate. A reaction flask containing methyl 2-(4-bromo-3-fluorophenyl)butanoate (1.02 g, 3.70 mmol), tert-butyl carbamate (531 mg, 4.44 mmol), palladium(II) acetate (83.0 mg, 370 umol), cesium carbonate (1.69 g, 5.18 mmol), and 2-dicyclohexylphosphino-2′,4′,6′-tri-iso-propyl-1,1′-biphenyl (529 mg, 1.11 mmol) was backfilled with nitrogen. Dioxane (31.9 mL), previously sparged with nitrogen, was then added.

The reaction mixture was stirred for 16 h at 95° C. The reaction mixture was concentrated under reduced pressure, and the resulting crude residue was purified by silica gel chromatography (0-30% EtOAc in hexanes) to afford ethyl 2-(4-((tert-butoxycarbonyl)amino)-3-fluorophenyl)butanoate as a yellow oil (1.2 g, 99% yield). LCMS [M+H-t-Bu]^{1=256.1.} ¹H NMR (CDCl₃, 400 MHz): δ 7.99 (t, J=8.2 Hz, 1H), 7.06-7.00 (m, 2H), 6.65 (s, 1H), 3.65 (s, 3H), 3.39 (t, J=7.7 Hz, 1H), 2.10-1.99 (m, 1H), 1.80-1.69 (m, 1H), 0.87 (t, J=7.4 Hz, 3H).

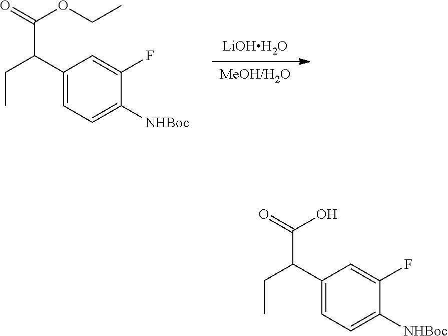 [SMILES: CCOC(=O)C(CC)c1ccc(NC(=O)OC(C)(C)C)c(F)c1 ; "LiOH·H2O" ; "MeOH/H2O" ; CCC(C(=O)O)c1ccc(NC(=O)OC(C)(C)C)c(F)c1]

2-(4-((tert-butoxycarbonyl)amino)-3-fluorophenyl)butanoic acid. To a solution of ethyl 2-(4-((tert-butoxycarbonyl)amino)-3-fluorophenyl)butanoate (1.21 g, 3.90 mmol) in a solution of methanol (73.9 mL) and water (22.4 mL) was added lithium hydroxide monohydrate (245 mg, 5.84 mmol). The reaction mixture was stirred for 16 h at RT. Additional lithium hydroxide monohydrate (245 mg, 5.84 mmol) was added, and the reaction mixture was stirred for another 16 h at RT. The reaction mixture was concentrated under reduced pressure. The residue was diluted with H₂O and adjusted pH=4 with HCl (2.00 M), extracted with DCM, dried over MgSO₄, filtered, and concentrated to afford 2-(4-((tert-butoxycarbonyl)amino)-3-fluorophenyl)butanoic acid (1.0 g, 88% yield) as a yellow solid.

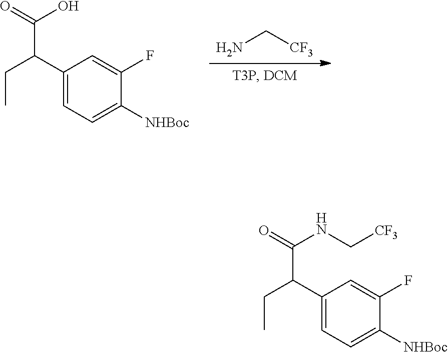

tert-butyl (2-fluoro-4-(1-oxo-1-((2,2,2-trifluoroethyl)amino)butan-2-yl)phenyl)carbamate.

To an ice-cold solution of 2-(4-((tert-butoxycarbonyl)amino)-3-fluorophenyl)butanoic acid (100 mg, 336 umol) in DCM (8.61 mL) were added consecutively 2,2,2-trifluoroethylamine (66.0 uL, 841 unol). N,N-diisopropylethylamine (205 uL, 1.18 mmol), and propyl phosphonic anhydride (23.2 uL, 39.0 umol). The reaction mixture was warmed to RT and stirred for 1 h. The reaction mixture was quenched with water and extracted with DCM. The solution was dried over MgSO₄, filtered, and concentrated under reduced pressure. The crude residue was purified by silica gel chromatography (0-100% EtOAc in hexanes) to afford tert-butyl (2-fluoro-4-(1-oxo-1-((2,2,2-trifluoroethyl)amino)butan-2-yl)phenyl)carbamate (70 mg, 55% yield) as a white solid. ¹H NMR (CDCl₃, 400 MHz): δ 8.05 (t, J=8.1 Hz, 1H), 7.05-6.98 (m, 2H), 6.68 (s, 1H), 5.61 (t, J=6.1 Hz, 1H), 4.02-3.89 (m, 1H), 3.83-3.71 (m, 1H), 3.22 (t, J=7.6 Hz, 1H), 2.20-2.09 (m, 1H), 1.81-1.70 (m, 1H), 1.52 (s, 9H), 0.87 (t, J=7.4 Hz, 3H).

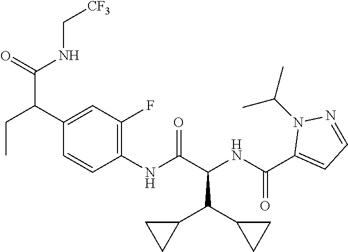 [SMILES: CCC(C(=O)NCC(F)(F)F)c1ccc(NC(=O)[C@@H](NC(=O)c2ccnn2C(C)C)C(C2CC2)C2CC2)c(F)c1]

N-((2S)-1,1-dicyclopropyl-3-((2-fluoro-4-(1-oxo-1-((2,2,2-trifluoroethyl)amino)butan-2-yl)phenyl)amino)-3-oxopropan-2-yl)-1-isopropyl-1H-pyrazole-5-carboxamide. Prepared according to General Scheme D, employing 2-(4-amino-2-cyclopropyl-5-fluorophenyl)-N-(2,2,2-trifluoroethyl)propanamide hydrochloride and (S)-2-((tert-butoxycarbonyl)amino)-3,3-dicyclopropylpropanoic acid followed by 1-isopropyl-1H-pyrazole-5-carboxylic acid. The title compound was isolated as the first eluting, single stereoisomer by chiral SFC purification (column: DAICEL CHIRALPAK IG (250 mm×10 mm, 5 um); mobile phase: 15% [MeOH). LCMS [M+H]⁺=566.4 m/z.

### Example 156: Synthesis of Compound 150

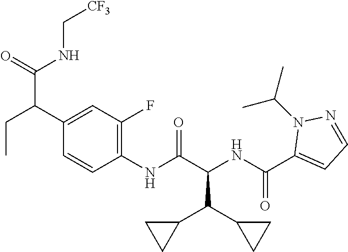 [SMILES: CCC(C(=O)NCC(F)(F)F)c1ccc(NC(=O)[C@@H](NC(=O)c2ccnn2C(C)C)C(C2CC2)C2CC2)c(F)c1]

N-((2S)-1,1-dicyclopropyl-3-((2-fluoro-4-(1-oxo-1-((2,2,2-trifluoroethyl)amino)butan-2-yl)phenyl)amino)-3-oxopropan-2-yl)-1-isopropyl-1H-pyrazole-5-carboxamide. Prepared according to Example 155. The title compound was isolated as the second eluting, single stereoisomer by chiral SFC purification (column: DAICEL CHIRALPAK IG (250 mm×10 mm, 5 um); mobile phase: 15% [MeOH). LCMS [M+H]⁺=566.4 m/z.

### Example 157: Synthesis of Compound 151

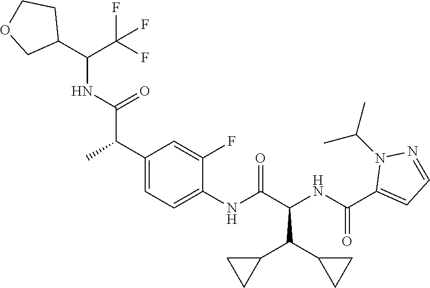 [SMILES: CC(C)n1nccc1C(=O)N[C@H](C(=O)Nc1ccc([C@H](C)C(=O)NC(C2CCOC2)C(F)(F)F)cc1F)C(C1CC1)C1CC1]

N-((2S)-1,1-dicyclopropyl-3-((2-fluoro-4-((2S)-1-oxo-1-((2,2,2-trifluoro-1-(tetrahydrofuran-3-yl)ethyl)amino)propan-2-yl)phenyl)amino)-3-oxopropan-2-yl)-1-ethyl-1H-pyrazole-5-carboxamide. Prepared according to General Scheme C, employing methyl (S)-2-(4-amino-3-fluorophenyl)propanoate and (S)-2-((tert-butoxycarbonyl)amino)-3,3-dicyclopropylpropanoic acid followed by 1-isopropyl-1H-pyrazole-5-carboxylic acid and 2,2,2-trifluoro-1-tetrahydrofuran-3-yl-ethanamine. The title compound was isolated as the first eluting isomer by silica gel chromatography, eluting with 0-100% EtOAc in hexanes. LCMS [M+H]⁺=622.2 m/z.

### Example 158: Synthesis of Compound 152

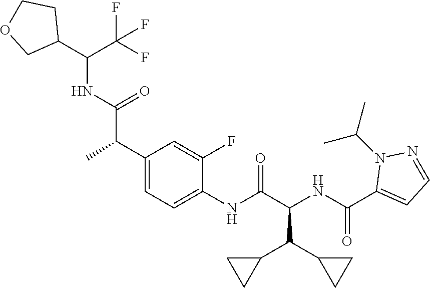 [SMILES: CC(C)n1nccc1C(=O)N[C@H](C(=O)Nc1ccc([C@H](C)C(=O)NC(C2CCOC2)C(F)(F)F)cc1F)C(C1CC1)C1CC1]

N-((2S)-1,1-dicyclopropyl-3-((2-fluoro-4-((2S)-1-oxo-1-((2,2,2-trifluoro-1-(tetrahydrofuran-3-yl)ethyl)amino)propan-2-yl)phenyl)amino)-3-oxopropan-2-yl)-1-ethyl-1H-pyrazole-5-carboxamide. Prepared according to General Scheme C (Example 157), employing methyl (S)-2-(4-amino-3-fluorophenyl)propanoate and (S)-2-((tert-butoxycarbonyl)amino)-3,3-dicyclopropylpropanoic acid followed by 1-isopropyl-1H-pyrazole-5-carboxylic acid and 2,2,2-trifluoro-1-tetrahydrofuran-3-yl-ethanamine. The title compound was isolated as the second eluting isomer by silica gel chromatography, eluting with 0-100% EtOAc in hexanes. LCMS [M+H]⁺=622.2 m/z.

### Example 159: Synthesis of Compound 153

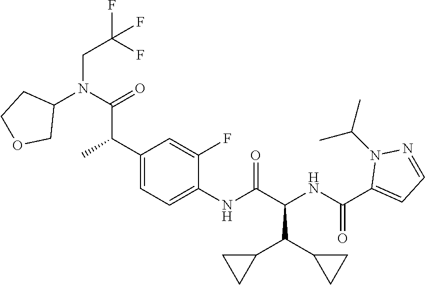 [SMILES: CC(C)n1nccc1C(=O)N[C@H](C(=O)Nc1ccc([C@H](C)C(=O)N(CC(F)(F)F)C2CCOC2)cc1F)C(C1CC1)C1CC1]

N-((2S)-1.1-dicyclopropyl-3-((2-fluoro-4-((2S)-1-oxo-1-((tetrahydrofuran-3-yl)(2,2,2-trifluoroethyl)amino)propan-2-yl)phenyl)amino)-3-oxopropan-2-yl)-1-ethyl-1H-pyrazole-5-carboxamide. Prepared according to General Scheme C, employing methyl (S)-2-(4-amino-3-fluorophenyl)propanoate and (S)-2-((tert-butoxycarbonyl)amino)-3,3-dicyclopropylpropanoic acid followed by 1-isopropyl-1H-pyrazole-5-carboxylic acid and N-(2,2,2-trifluoroethyl)tetrahydrofuran-3-amine hydrochloride. LCMS [M+H]⁺=622.2 m/z.

### Example 160: Synthesis of Compound 154

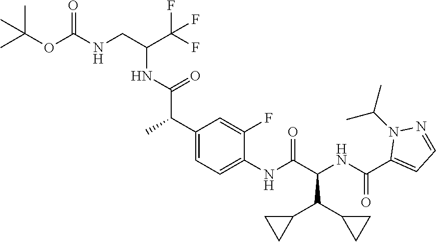 [SMILES: CC(C)n1nccc1C(=O)N[C@H](C(=O)Nc1ccc([C@H](C)C(=O)NC(CNC(=O)OC(C)(C)C)C(F)(F)F)cc1F)C(C1CC1)C1CC1]

tert-butyl (2-((S)-2-(4-((S)-3,3-dicyclopropyl-2-(1-ethyl-1H-pyrazole-5-carboxamido)propanamido)-3-fluorophenyl)propanamido)-3,3,3-trifluoropropyl)carbamate. Prepared according to General Scheme C, employing methyl (S)-2-(4-amino-3-fluorophenyl)propanoate and (S)-2-((tert-butoxycarbonyl)amino)-3,3-dicyclopropylpropanoic acid followed by 1-isopropyl-1H-pyrazole-5-carboxylic acid and tert-butyl N-(2-amino-3,3,3-trifluoro-propyl)carbamate. LCMS [M+H]⁺=681.2 m/z.

### Example 161: Synthesis of Compound 155

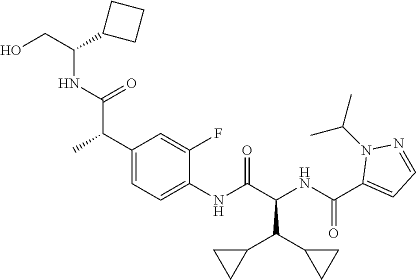 [SMILES: CC(C)n1nccc1C(=O)N[C@H](C(=O)Nc1ccc([C@H](C)C(=O)N[C@H](CO)C2CCC2)cc1F)C(C1CC1)C1CC1]

N-((S)-1-((4-((S)-1-(((S)-1-cyclobutyl-2-hydroxyethyl)amino)-1-oxopropan-2-yl)-2-fluorophenyl)amino)-3.3-dicyclopropyl-1-oxopropan-2-yl)-1-ethyl-1H-pyrazole-5-carboxamide. Prepared according to General Scheme C, employing methyl (S)-2-(4-amino-3-fluorophenyl)propanoate and (S)-2-((tert-butoxycarbonyl)amino)-3,3-dicyclopropylpropanoic acid followed by 1-isopropyl-1H-pyrazole-5-carboxylic acid and (2S)-2-amino-2-cyclobutyl-ethanol hydrochloride. LCMS JM+HI =568.2 m/z.

### Example 162: Synthesis of Compound 156

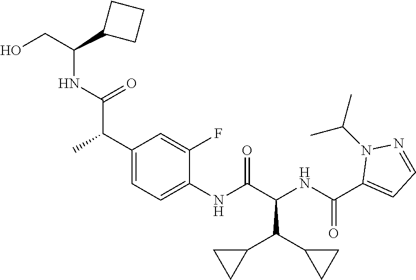 [SMILES: CC(C)n1nccc1C(=O)N[C@H](C(=O)Nc1ccc([C@H](C)C(=O)N[C@@H](CO)C2CCC2)cc1F)C(C1CC1)C1CC1]

N-((S)-1-((4-((S)-1-(((R)-1-cyclobutyl-2-hydroxyethyl)amino)-1-oxopropan-2-yl)-2-fluorophenyl)amino)-3,3-dicyclopropyl-1-oxopropan-2-yl)-1-ethyl-1H-pyrazole-5-carboxamide. Prepared according to General Scheme C, employing methyl (S)-2-(4-amino-3-fluorophenyl)propanoate and (S)-2-((tert-butoxycarbonyl)amino)-3,3-dicyclopropylpropanoic acid followed by 1-isopropyl-1H-pyrazole-5-carboxylic acid and (2R)-2-amino-2-cyclobutyl-ethanol hydrochloride. LCMS [M+H]⁺=568.2 m/z.

### Example 163: Synthesis of Compound 157

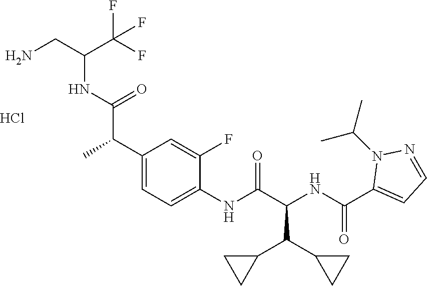 [SMILES: Cl ; CC(C)n1nccc1C(=O)N[C@H](C(=O)Nc1ccc([C@H](C)C(=O)NC(CN)C(F)(F)F)cc1F)C(C1CC1)C1CC1]

N-((2S)-1-((4-((2S)-1-((3-amino-1,1,1-trifluoropropan-2-yl)amino)-1-oxopropan-2-yl)-2-fluorophenyl)amino)-3.3-dicyclopropyl-1-oxopropan-2-yl)-1-isopropyl-1H-pyrazole-5-carboxamide hydrochloride. Prepared according to General Procedure C, employing tert-butyl (2-((S)-2-(4-((S)-3,3-dicyclopropyl-2-(1-ethyl-1H-pyrazole-5-carboxamido)propanamido)-3-fluorophenyl)propanamido)-3,3,3-trifluoropropyl)carbamate. LCMS [M+H]⁺=581.1 m/z.

### Example 164: General Scheme G-Synthesis of Compounds 158-164

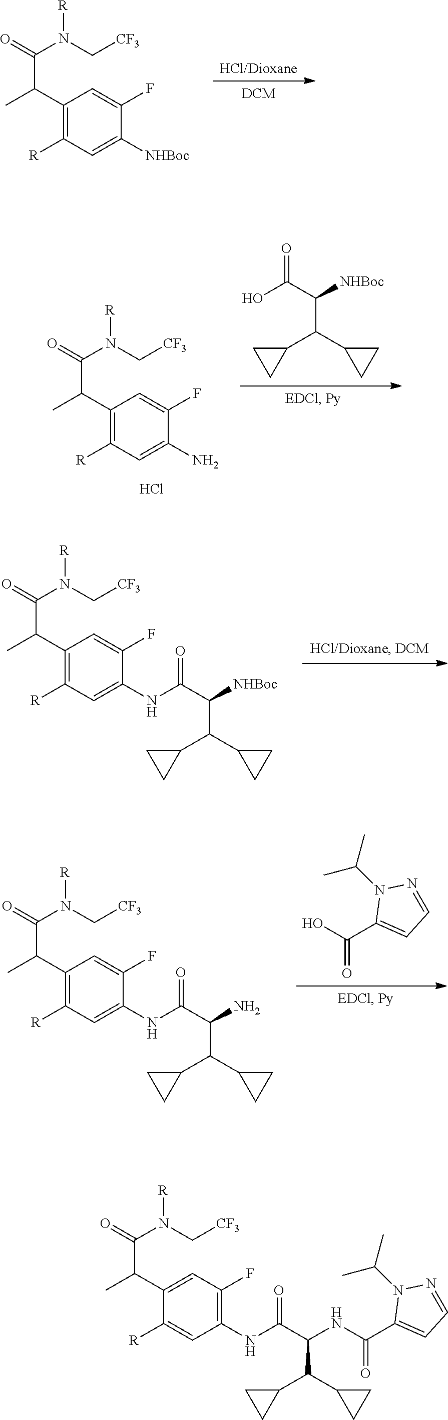

### Example 165: Synthesis of Compound 158

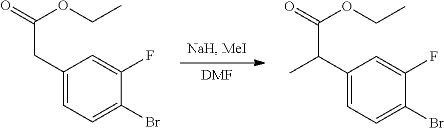

To the solution of ethyl 2-(4-bromo-3-fluorophenyl)acetate (10.0 g, 38.3 mmol, 1.00 eq) in DMF (80.0 mL) was added NaH (1.61 g, 40.2 mmol, 60.0% purity, 1.05 eq) at 0° C. The reaction mixture was stirred at 0° C. for 1 h. Then Mel (5.71 g, 40.2 mmol, 2.50 mL, 1.05 eq) in DMF (20.0 mL) was added and stirred at RT for 4 h. The reaction mixture was diluted with aq. NH₄Cl and extracted with EtOAc. The organic layer was washed with brine, dried over anhydrous Na₂SO₄, filtered, and concentrated under vacuum to afford ethyl 2-(4-bromo-3-fluorophenyl)propanoate (10.6 g, crude) as a yellow oil. ¹H NMR (400 MHz, DMSO-d6) δ 7.67-7.64 (m, 1H), 7.33-7.30 (m, 1H), 7.11-7.08 (m, 1H), 4.09-4.05 (m, 2H), 4.04-3.83 (m, 1H), 1.38 (d, J=7.2 Hz, 3H), 1.14-1.11 (m, 3H).

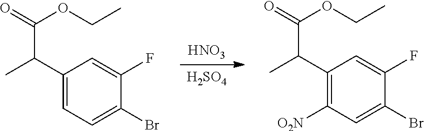

To the solution of ethyl 2-(4-bromo-3-fluorophenyl)propanoate (10.6 g, 38.5 mmol, 1.00 eq) in H₂SO₄ (50.0 mL) was added HNO₃ (4.49 g, 71.2 mmol, 3.21 mL, 1.85 eq) at 0° C. The mixture was stirred at 0° C. for 1 h. The reaction mixture was poured into 200 mL of H₂O at 0° C. and extracted with EtOAc. The organic layer was washed with H₂O, dried over anhydrous Na₂SO₄, filtered, and concentrated under vacuum to give a residue. The residue (was purified by column chromatography (SiO₂, pet-ether:EtOAc=100:1 to 10:1) to afford ethyl 2-(4-bromo-5-fluoro-2-nitrophenyl)propanoate (8.80 g, 27.4 mmol, 71.3% yield) as a yellow oil. ¹H NMR (400 MHz, CDCl₃) δ 8.24 (d, J=6.0 Hz, 1H), 7.27 (d, J=1 1.2 H, 1H), 4.34-4.18 (m, 1H), 4.17-4.12 (m, 2H), 1.58 (d, J=6.0 Hz, 3H), 1.24-1.20 (m, 3H).

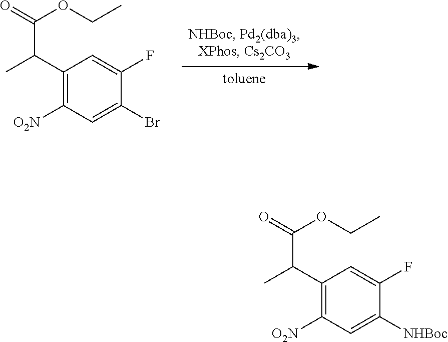 [SMILES: CCOC(=O)C(C)c1cc(F)c(Br)cc1[N+](=O)[O-] ; "NHBoc, Pd2(dba)3,XPhos, Cs2CO3" ; "toluene" ; CCOC(=O)C(C)c1cc(F)c(NC(=O)OC(C)(C)C)cc1[N+](=O)[O-]]

To the solution of ethyl 2-(4-bromo-5-fluoro-2-nitrophenyl)propanoate (8.80 g, 27.4 mmol, 1.00 eq) and NH₂Boc (4.83 g, 41.2 mmol, 1.50 eq) in toluene (60.0 mL) was added Pd₂(dba)₃ (2.52 g, 2.75 mmol, 0.100 eq), XPhos (2.62 g, 5.50 mmol, 0.200 eq), and Cs₂CO₃ (26.8 g, 82.4 mmol, 3.00 eq). The mixture was stirred at 100° C. for 1 h. The reaction mixture was diluted with H₂O, extracted with EtOAc, filtered, and concentrated under vacuum to give a residue. The residue was purified by column chromatography (SiO₂, pet-ether:EtOAc=100:1 to 10:1) to afford ethyl 2-(4-((tert-butoxycarbonyl)amino)-5-fluoro-2-nitrophenyl)propanoate (8.40 g, 23.5 mmol, 85.7% yield) as a yellow oil. ¹H NMR (400 MHz, CDCl₃). δ 8.81 (d, J=7.2 Hz, 1H), 7.12-7.08 (m, 1H), 6.79 (s, 1H), 4.30-4.16 (m, 1H), 4.14-4.10 (m, 2H), 1.59-1.54 (m, 12H), 1.21 (t, J=7.2 Hz, 3H)

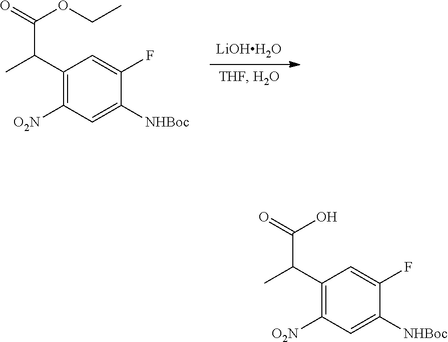

To the solution of ethyl 2-(4-((tert-butoxycarbonyl)amino)-5-fluoro-2-nitrophenyl)propanoate (8.40 g, 23.5 mmol, 1.00 eq) in THF (80.0 mL) was added LiOH H₂O (5.94 g, 141 mmol, 6.0) eq) in H₂O (20.0 mL) at 0° C. The mixture was stirred at rt for 2 h. The reaction mixture was adjusted to pH=3 with 1 M HCl, extracted with EtOAc, filtered, and concentrated under vacuum to give a residue. The residue was purified by column chromatography (SiO₂, pet-ether:EtOAc=100:1 to 1:3) to afford 2-(4-((tert-butoxycarbonyl)amino)-5-fluoro-2-nitrophenyl)propanoic acid (5.95 g, 18.1 mmol, 76.8% yield) as a yellow solid. ¹H NMR (400 MHz, DMSO-d₆). δ 12.5 (s, 1H), 9.54 (s, 1H), 8.43 (d, J=2.0 Hz, 1H), 7.45 (d, J=11.6 Hz, 1H), 4.20-4.11 lH 1.49-1.45 m 12H).

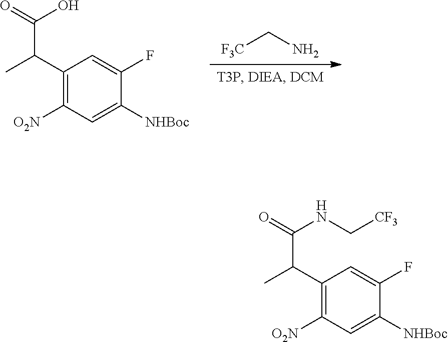

To the solution of 2-(4-((tert-butoxycarbonyl)amino)-5-fluoro-2-nitrophenyl)propanoic acid (5.95 g, 18.1 mmol, 1.00 eq) and 2,2,2-trifluoroethan-1-amine (2.69 g, 27.1 mmol, 2.14 mL, 1.50 eq) in DCM (20.0 mL) was added DIEA (11.7 g, 90.6 mmol, 15.7 mL, 5.00 eq) and T3P (34.6 g, 54.3 mmol, 32.3 mL, 50.0% purity, 3.00 eq) at 0° C. The mixture was stirred at RT for 2 h. The reaction mixture was diluted with H₂O and extracted with DCM. The organic layer was washed with sat. NH₄Cl, dried over anhydrous Na₂SO₄, filtered, and concentrated under vacuum to afford tert-butyl (2-fluoro-5-nitro-4-(1-oxo-1-((2,2,2-trifluoroethyl)amino)propan-2-yl)phenyl)carbamate (7.00 g, 17.1 mmol, 94.3% yield) as a yellow solid. ¹H NMR (400 MHz, CDCl₃) δ 8.75 (d, J=7.2 Hz, 1H), 7.34 (d, J=11.6 Hz, 1H), 6.82 (s, 1H), 6.45-6.42 (m, 1H), 4.16-4.14 (m, 1H), 3.93-3.84 (m, 2H), 1.50 (d, J=7.2 Hz, 9H), 1.49-1.42 (m, 3H).

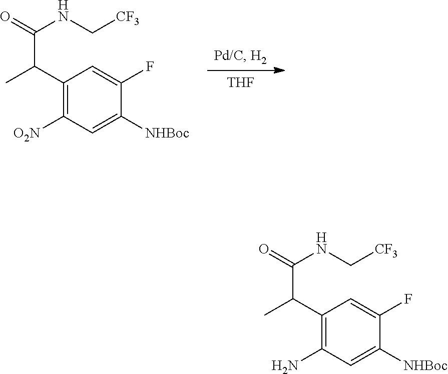 [SMILES: CC(C(=O)NCC(F)(F)F)c1cc(F)c(NC(=O)OC(C)(C)C)cc1[N+](=O)[O-] ; "Pd/C, H2" ; CC(C(=O)NCC(F)(F)F)c1cc(F)c(NC(=O)OC(C)(C)C)cc1N ; "THF"]

To a solution of tert-butyl (2-fluoro-5-nitro-4-(1-oxo-1-((2,2,2-trifluoroethyl)amino)propan-2-yl)phenyl)carbamate (7.00 g, 17.1 mmol, 1.00 eq) in THF (30.0 mL) was added Pd/C (100 mg, 17.1 mmol, 10.0% purity, 1.00 eq) under N₂. The suspension was degassed under vacuum and purged with H₂ several times. The reaction mixture was stirred under H₂ (15.0 psi) at RT for 2 h. The reaction mixture was filtered and concentrated under vacuum to give a residue. The residue was purified by column chromatography (SiO₂, pet-ether:EtOAc=100:1 to 2:1) to afford tert-butyl (5-amino-2-fluoro-4-(1-oxo-1-((2,2,2-trifluoroethyl)amino)propan-2-yl)phenyl)carbamate (1.80 g, 4.74 mmol, 27.7% yield) as a yellow solid. LCMS [M+H] 380.1 m. ¹H NMR (400 MHz, CDCl₃) δ 7.57 (d, J=7.2 Hz, 1H), 6.88 (d,I=12 Hz, 1H), 6.67 (s, 1H), 6.36 (s, 1H), 3.86-3.80 (m, 2H), 3.78-3.59 (m, 1H), 1.53-1.50 (m, 12H).

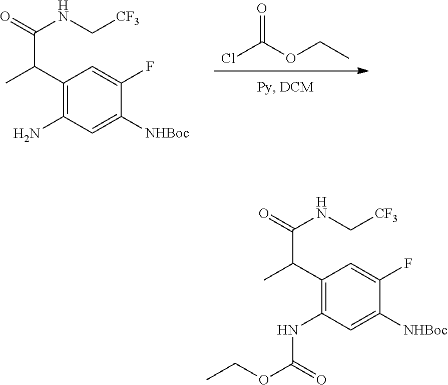

To the solution of tert-butyl (5-amino-2-fluoro-4-(1-oxo-1-((2,2,2—PGP454,C₃ trifluoroethyl)amino)propan-2-yl)phenyl)carbamate (200 mg, 527 umol, 1.00 eq) and pyridine (83.4 mg, 1.05 mmol, 85.1 uL, 2.00 eq) in DCM (10.0 mL) was added ethyl carbonochloridate (470 mg, 4.33 mmol, 412 μL, 8.21 eq) at 0° C. The mixture was stirred at RT for 2 h. The reaction was diluted with sat. NH₄Cl and extracted with DCM. The organic layer was washed with sat. NH₄Cl, dried over anhydrous Na₂SO₄, filtered, and concentrated under vacuum to afford tert-butyl ethyl (4-fluoro-6-(1-oxo-1-((2,2,2-trifluoroethyl)amino)propan-2-yl)-1,3-phenylene)dicarbamate (350 mg, crude) as a yellow solid. LCMS [M+H]⁺=452.2 m/z.

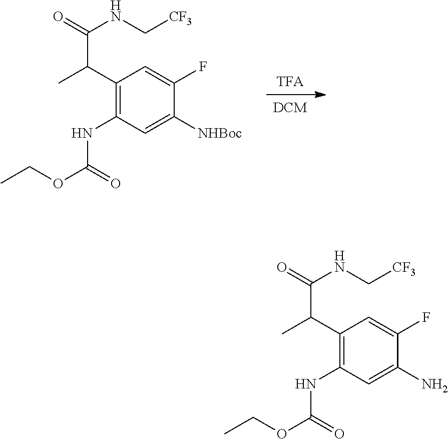

To the solution of tert-butyl ethyl (4-fluoro-6-(1-oxo-1-((2,2,2-trifluoroethyl)amino)propan-2-yl)-1,3-phenylene)dicarbamate (350 mg, 775 umol, 1.00 eq) in DCM (5.00 mL) was added TFA (442 mg, 3.88 mmol, 287 μL, 5.00 eq) at 0° C. The mixture was stirred at RT for 2 h. The reaction mixture was diluted with sat. aq NaHCO₃, extracted with DCM, filtered, and concentrated under vacuum to afford ethyl (5-amino-4-fluoro-2-(1-oxo-1-((2,2,2-trifluoroethyl)amino)propan-2-yl)phenyl)carbamate (280 mg, crude) as a yellow solid. LCMS [M+H]⁺=352.3 m/z.

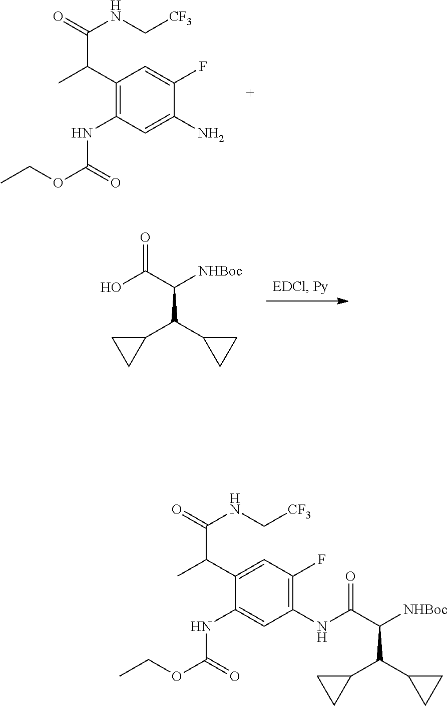 [SMILES: CCOC(=O)Nc1cc(N)c(F)cc1C(C)C(=O)NCC(F)(F)F ; CC(C)(C)OC(=O)N[C@H](C(=O)O)C(C1CC1)C1CC1 ; "EDCl, Py" ; CCOC(=O)Nc1cc(NC(=O)[C@@H](NC(=O)OC(C)(C)C)C(C2CC2)C2CC2)c(F)cc1C(C)C(=O)NCC(F)(F)F]

To a solution of ethyl (5-amino-4-fluoro-2-(1-oxo-1-((2,2,2-trifluoroethyl)amino)propan-2-yl)phenyl)carbamate (240 mg, 683 umol, 1.00 eq) and (S)-2-((tert-butoxycarbonyl)amino)-3,3-dicyclopropylpropanoic acid (202 mg, 751 umol, 1.10 eq) in pyridine (1.00 mL) was added EDCI (523 mg, 2.73 mmol, 4.00 eq). The reaction mixture was stirred at RT for 14 h. The reaction mixture was diluted with H₂O and extracted with EtOAc. The combined organic layers were washed with sat aq. NaCl, dried over Na₂SO₄, filtered, and concentrated under reduced pressure to give a residue. The residue was purified by prep-TLC followed by prep-HPLC (column: Unisil 3-100 C18 Ultra 150 * 50 mm * 3 um) eluting with water with 0.1% formic acid and ACN (41-71% ACN) to afford ethyl (5-((S)-2-((tert-butoxycarbonyl)amino)-3,3-dicyclopropylpropanamido)-4-fluoro-2-(1-oxo-1-((2,2,2-trifluoroethyl)amino)propan-2-yl)phenyl)carbamate (200 mg, 331 umol, 48.5% yield) as a yellow solid.

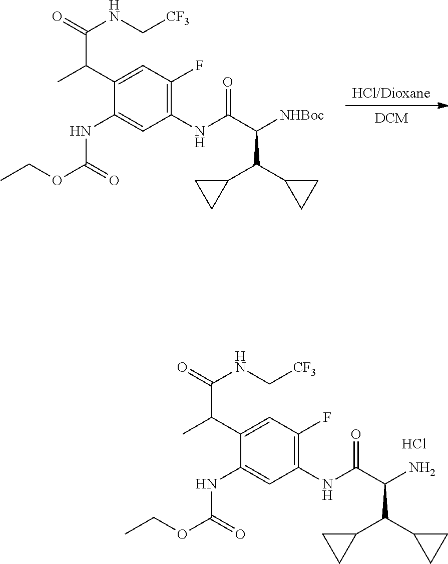

To a solution of ethyl (5-((S)-2-((tert-butoxycarbonyl)amino)-3,3dicyclopropylpropanamido)-4-fluoro-2-(1-oxo-1-((2,2,2-trifluoroethyl)amino)propan-2-yl)phenyl)carbamate (100 mg, 165 umol, 1.00 eq) in DCM (2.00 mL) was added HCl/dioxane (4 M, 6.67 mL, 160 eq) at 0° C. The mixture was stirred at RT for 3 h. The reaction mixture was concentrated under reduced pressure to afford ethyl (5-((S)-2-amino-3,3-dicyclopropylpropanamido)-4-fluoro-2-(1-oxo-1-((2,2,2-trifluoroethyl)amino)propan-2-yl)phenyl)carbamate (90.0 mg, crude, HCl) as yellow solid.

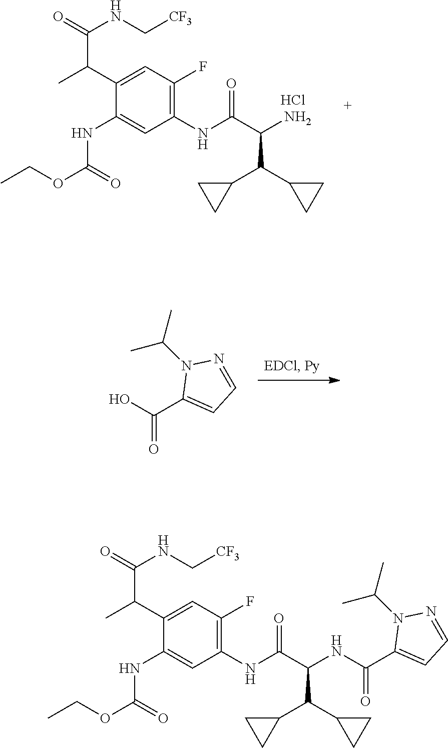 [SMILES: CCOC(=O)Nc1cc(NC(=O)[C@@H](N)C(C2CC2)C2CC2)c(F)cc1C(C)C(=O)NCC(F)(F)F ; Cl ; CC(C)n1nccc1C(=O)O ; "EDCl, Py" ; CCOC(=O)Nc1cc(NC(=O)[C@@H](NC(=O)c2ccnn2C(C)C)C(C2CC2)C2CC2)c(F)cc1C(C)C(=O)NCC(F)(F)F]

To a solution of ethyl (5-((S)-2-amino-3,3-dicyclopropylpropanamido)-4-fluoro-2-(1-oxo-1-((2,2,2-trifluoroethyl)amino)propan-2-yl)phenyl)carbamate (90.0 mg, 166 umol, 1.00 eq. HCl) and 1-isopropyl-1H-pyrazole-5-carboxylic acid (28.3 mg, 183 umol, 1.10 eq) in pyridine (4.00 mL) was added EDCI (160 mg, 834 umol, 5.( ) eq). The mixture was stirred at RT for 12 h. The residue was diluted with H₂O. extracted with EtOAc, dried over Na₂SO₄. filtered, and concentrated under reduced pressure to give a residue. The residue was purified by prep-HPLC (column: Unisil 3-100 C18 Ultra 150 * 50 mm * 3 um; mobile phase: [water (FA)−ACN]; 37%-67%) to afford the desired product (60 mg, 93.9 umol, 56.2% yield) as yellow solid. The mixture was purified by prep-HPLC (column: DAICEL CHIRALPAK AD (250 mm * 30 mm, 10 um); mobile phase: NH·H₂O IPA]; B %: 15%) to afford the desired product as the second eluting, single stereoisomer ethyl (5-((S)-3,3-dicyclopropyl-2-(1-isopropyl-1H-pyrazole-5-carboxamido)propanamido)-4-fluoro-2-(1-oxo-1-((2,2,2-trifluoroethyl)amino)propan-2-yl)phenyl)carbamate (25.3 mg, 38.5 umol, 41.0% yield) as a white solid. LCMS [M+H]⁺=639.3 m/z.

### Example 166: Synthesis of Compound 159

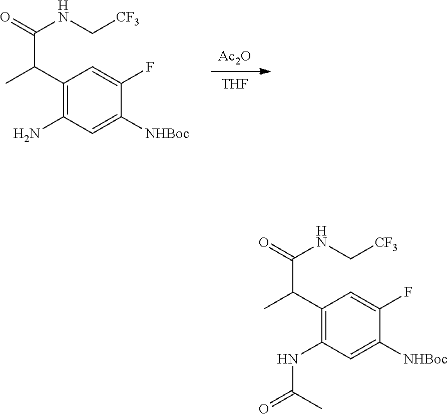 [SMILES: "Ac2O" ; CC(C(=O)NCC(F)(F)F)c1cc(F)c(NC(=O)OC(C)(C)C)cc1N ; CC(=O)Nc1cc(NC(=O)OC(C)(C)C)c(F)cc1C(C)C(=O)NCC(F)(F)F ; "THF"]

To a solution of tert-butyl (5-amino-2-fluoro-4-(1-oxo-1-((2,2,2-trifluoroethyl)amino)propan-2-yl)phenyl)carbamate (200 mg, 527 umol, 1.00 eq) in DCM (10.0 mL) was added TEA (160 mg, 1.58 mmol, 220 μL, 3.00 eq) and Ac₂O (80.7 mg, 791 umol, 74.1 uL, 1.50 eq). The reaction mixture was stirred at RT for 2 h. The reaction mixture was poured over 10.0 mL H₂O and extracted with DCM. The combined organic layers were washed with sat.aq NH₄C₁, dried over Na₂SO₄. filtered, and concentrated under reduced pressure to give a residue. The residue was purified by prep-TLC (SiO₂, pet-ether:ethyl acetate=1:1) to afford tert-butyl (5-acetamido-2-fluoro-4-(1-oxo-1-((2,2,2-trifluoroethyl)amino)propan-2-yl)phenyl)carbamate (139 mg, 330 umol, 62.5% yield) as a white solid. ¹H NMR (400 MHz, CDCl₃) δ 7.97 (d, J=7.2 Hz, 1H), 7.64 (s, 1H), 7.31 (s, 1H), 7.07-7.04 (m, 1H), 6.73 (s, 1H), 3.90-3.82 (m, 2H), 3.80-3.64 (m, 1H), 2.23 (s, 3H), 1.52 s, 9H), 1.34 (d, J=6.8 Hz, 3H).

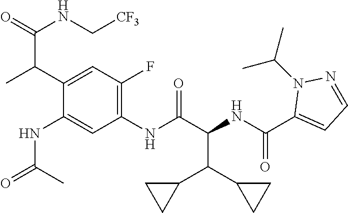 [SMILES: CC(=O)Nc1cc(NC(=O)[C@@H](NC(=O)c2ccnn2C(C)C)C(C2CC2)C2CC2)c(F)cc1C(C)C(=O)NCC(F)(F)F]

N-((2S)-1-((5-acetamido-2-fluoro-4-(1-oxo-1-((2,2,2-trifluoroethyl)amino)propan-2-yl)phenyl)amino)-3,3-dicyclopropyl-1-oxopropan-2-yl)-1-isopropyl-1H-pyrazole-5-carboxamide. Prepared according to General Scheme G, employing (S)-2-((tert-butoxycarbonyl)amino)-3,3-dicyclopropylpropanoic acid and tert-butyl (5-acetamido-2-fluoro-4-(1-oxo-1-((2,2,2-trifluoroethyl)amino)propan-2-yl)phenyl)carbamate followed by 1-isopropyl-1H-pyrazole-5-carboxylic acid. The desired intermediate (2S)—N-(5-acetamido-2-fluoro-4-(1-oxo-1-((2,2,2-trifluoroethyl)amino)propan-2-yl)phenyl)-2-amino-3,3-dicyclopropylpropanamide was isolated as the first eluting, single stereoisomer by chiral SFC (column: DAICEL CHIRALPAK IG (250 mm * 30 mm, 10 um), mobile phase: [0.1% NH₃H₂O ETOH]; 20%). LCMS [M+H]⁺=609.4 m/z.

### Example 167: Synthesis of Compound 160

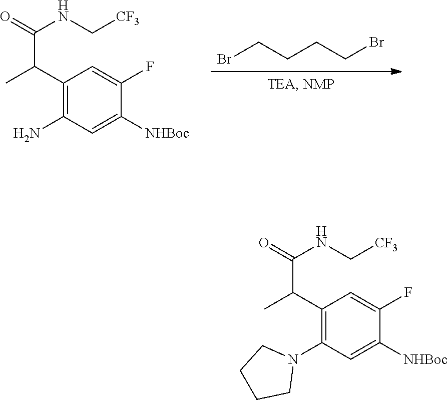

A microwave reaction tube was charged with tert-butyl (5-amino-2-fluoro-4-(1-oxo-1-((2,2,2-trifluoroethyl)amino)propan-2-yl)phenyl)carbamate (20.0 mg, 52.7 umol, 1.00 eq), 1,4-dibromobutane (34.1 mg, 158 umol, 19.0 uL, 3.00 eq), and TEA (16.0 mg, 158 umol, 22.0 uL, 3.00 eq) and diluted with NMP (4.00 mL). The sealed tube was heated at 150° C. for 1 h under microwave. The reaction mixture was diluted with H₂O and extracted with ethyl acetate. The combined organic layers were washed with brine, dried over Na₂SO₄, filtered, and concentrated under reduced pressure to give a residue. The residue was purified by prep-TLC (SiO₂, pet-ether:ethyl acetate=3:1) to afford tert-butyl (2-fluoro-4-(1-oxo-1-((2,2,2-trifluoroethyl)amino)propan-2-yl)-5-(pyrrolidin-1-yl)phenyl)carbamate (45.0 mg, 103 umol, 32.8% yield) as a yellow oil. LCMS [M+H]⁺=434.2 m/z.

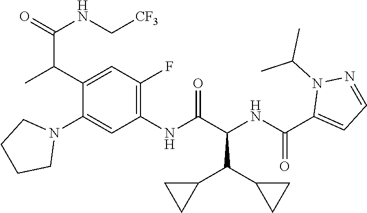 [SMILES: CC(C(=O)NCC(F)(F)F)c1cc(F)c(NC(=O)[C@@H](NC(=O)c2ccnn2C(C)C)C(C2CC2)C2CC2)cc1N1CCCC1]

N-((2S)-1,1-dicyclopropyl-3-((2-fluoro-4-(1-oxo-1-((2,2,2-trifluoroethyl)amino)propan-2-yl)-5-(pyrrolidin-1-yl)phenyl)ammno)-3-oxopropan-2-yl)-1-isopropyl-1H-pyrazole-5-carboxamide. Prepared according to General Scheme G, employing (S)-2-((tert-butoxycarbonyl)amino)-3,3-dicyclopropylpropanoic acid and tert-butyl (2-fluoro-4-(1-oxo-1-((2,2,2-trifluoroethyl)amino)propan-2-yl)-5-(pyrrolidin-1-yl)phenyl)carbamate followed by 1-isopropyl-1H-pyrazole-5-carboxylic acid. LCMS [M+H]⁺=621.4 m/z.

### Example 168: Synthesis of Compound 16

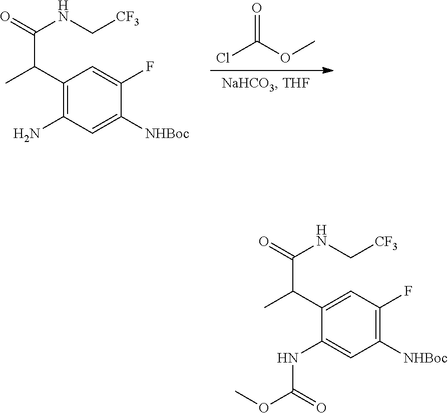

To a solution of tert-butyl (5-amino-2-fluoro-4-(1-oxo-1-((2,2,2-trifluoroethyl)amino)propan-2-yl)phenyl)carbamate (300 mg, 791 umol, 1.00 eq) in DCM (10.0 mL) was added NaHCO₃ (199 mg, 2.37 mmol, 3.00 eq) and methyl carbonochloridate (112 mg, 1.19 mmol, 91.9 uL, 1.50 eq) at 0° C. The mixture was stirred at RT for 1 h. The reaction mixture was diluted with water and extracted with EtOAc. The combined organic layer was washed with brine, dried over Na₂SO₄, filtered, and concentrated under reduced pressure to afford tert-butyl methyl (4-fluoro-6-(1-oxo-1-((2,2,2-trifluoroethyl)amino)propan-2-yl)-1,3-phenylene)dicarbamate (300 mg, 685 umol, 86.7% yield) as a yellow solid. ¹H NMR (400 MHz, CDCl₃) δ 8.03 (d, J=7.6 Hz, 1H), 7.43 (brs, 1H), 7.04 (d, J=12.0 Hz, 1H), 6.74-6.73 (m, 2H), 3.96-3.85 (m, 1H), 3.78 (s, 3H), 3.77-3.70 (m, 2H), 1.51 (s, 9H), 1.35 (d, J=6.8 Hz, 3H).

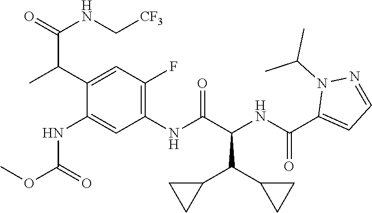 [SMILES: COC(=O)Nc1cc(NC(=O)[C@@H](NC(=O)c2ccnn2C(C)C)C(C2CC2)C2CC2)c(F)cc1C(C)C(=O)NCC(F)(F)F]

methyl (5-((S)-3,3-dicyclopropyl-241-isopropyl-1H-pyrazole-5-carboxamido)propanamido)-4-fluoro-2-(1-oxo-1-((2,2,2-trifluoroethyl)amino)propan-2-yl)phenyl)carbamate. Prepared according to General Scheme G, employing (S)-2-((tert-butoxycarbonyl)amino)-3,3-dicyclopropylpropanoic acid and tert-butyl (2-fluoro-4-(1-oxo-1-((2,2,2-trifluoroethyl)amino)propan-2-yl)-5-(pyrrolidin-1-yl)phenyl)carbamate followed by 1-isopropyl-1H-pyrazole-5-carboxylic acid. The title compound was purified by chiral SFC to afford the first eluting, single stereoisomer (column: REGIS(S,S)WHELK-O1(250 mm * 25 mm, 10 um); mobile phase: [0.1% NH₃H₂O IPA]; 25%). LCMS [M+H]⁺=625.4 m/z.

### Example 169: Synthesis of Compound 162

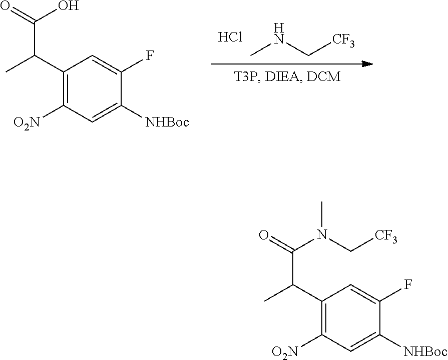

To a solution of 2-(4-((tert-butoxycarbonyl)amino)-5-fluoro-2-nitrophenyl)propanoic acid (500 mg, 1.52 mmol, 1.00 eq) and 2,2,2-trifluoro-N-methylethan-1-amine (296 mg, 1.98 mmol, 1.30 eq, HCl) in DCM (4.00 mL) was added T3P (1.45 g, 2.28 mmol, 1.36 mL, 50.0% purity, 1.50 eq) and DIEA (984 mg, 7.62 mmol, 1.33 mL, 5.00 eq) at 0° C. The reaction mixture was stirred at RT for 12 h. The reaction mixture was concentrated under reduced pressure, and the resulting residue was purified by prep-HPLC (column: Phenomenex luna C18 150 * 40 mm * 15 um; mobile phase: [water (FA)−ACN]; 48%-78%) to afford tert-butyl (2-fluoro-4-(1-(methyl(2,2,2-trifluoroethyl)amino)-1-oxopropan-2-yl)-5-nitrophenyl)carbamate (320 mg, crude) as a yellow oil. ¹H NMR (400 MHz, CDCl₃) δ 8.82 (d, J=6.0 Hz, 1H), 7.23 (d, J=11 Hz, 1H), 6.81 (s, 1H), 4.67-4.62 (m, 1H), 4.16-3.95 (m, 2H), 3.08 (s, 3H), 1.54 (s, 9H), 1.48 (d, J=7.2 Hz, 3H).

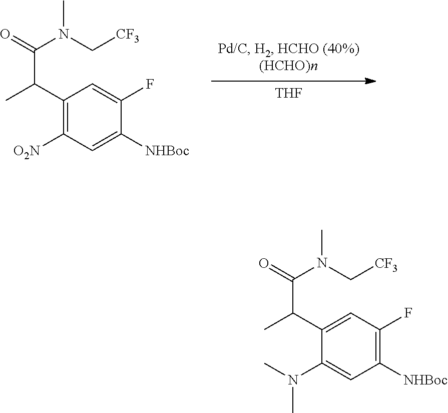 [SMILES: CC(C(=O)N(C)CC(F)(F)F)c1cc(F)c(NC(=O)OC(C)(C)C)cc1[N+](=O)[O-] ; "Pd/C, H2, HCHO (40%)(HCHO)n" ; CC(C(=O)N(C)CC(F)(F)F)c1cc(F)c(NC(=O)OC(C)(C)C)cc1N(C)C ; "THF"]

To a solution of tert-butyl (2-fluoro-4-(1-(methyl(2,2,2-trifluoroethyl)amino)-1-oxopropan-2-yl)-5-nitrophenyl)carbamate (300 mg, 710 unol, 1.00 eq), (HCHO)n (80.0 mg, 30.5 uL) and HCHO (2.18 g, 26.8 mmol, 2.00 mL, 37.0% purity, 37.8 eq) in THF (10.0 mL) was added Pd/C (50.0 mg, 10.0% purity) under N₂. The suspension was degassed under vacuum and purged with H₂ three times. The mixture was stirred under H₂ (15.0 psi) at RT for 30 h. The mixture was filtered, rinsing with MeOH, and the filtrate was concentrated to give a residue. The residue was purified by prep-TLC (SiO₂, pet-ether:EtOAc=5:1) to afford tert-butyl (5-(dimethylamino)-2-fluoro-4-(I-(methyl(2,2,2-trifluoroethyl)amino)-1-oxopropan-2-yl)phenyl)carbamate (220 mg, crude) as a yellow oil. ¹H NMR (400 MHz, CDCl₃) δ 7.98-7.93 (m, 1H), 7.11-6.99 (m, 1H), 6.67 (s, 1H), 4.85-4.42 (m, 1H), 4.00-3.97 (m, 1H), 3.33-3.30 (m, 1H), 3.07-2.92 (m, 3H), 2.69 (s, 6H), 1.53 (s, 9H), 1.45-1.42 (m, 3H).

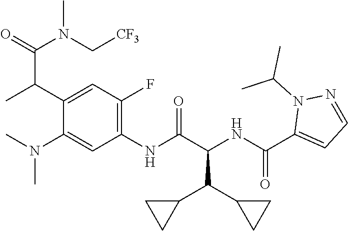 [SMILES: CC(C(=O)N(C)CC(F)(F)F)c1cc(F)c(NC(=O)[C@@H](NC(=O)c2ccnn2C(C)C)C(C2CC2)C2CC2)cc1N(C)C]

N-((2S)-1,1-dicyclopropyl-3-((5-(dimethylamino)-2-fluoro-4-(1-(methyl(2,2,2-trifluoroethyl)amino)-1-oxopropan-2-yl)phenyl)amino)-3-oxopropan-2-yl)-1-isopropyl-1H-pyrazole-5-carboxamide. Prepared according to General Scheme G, employing (S)-2-((tert-butoxycarbonyl)amino)-3,3-dicyclopropylpropanoic acid and tert-butyl (5-(dimethylamino)-2-fluoro-4-(I-(methyl(2,2,2-trifluoroethyl)amino)-1-oxopropan-2-yl)phenyl)carbamate followed by 1-isopropyl-1H-pyrazole-5-carboxylic acid. The title compound was purified by chiral SFC to afford the first eluting, single stereoisomer (column: DAICEL CHIRALPAK IC (250 mm * 30 mm, 5 um); mobile phase: [0.1% NNH₃H₂O in IPA]; 20%). LCMS [M+H]⁺=609.4 m/z.

### Example 170: Synthesis of Compound 163

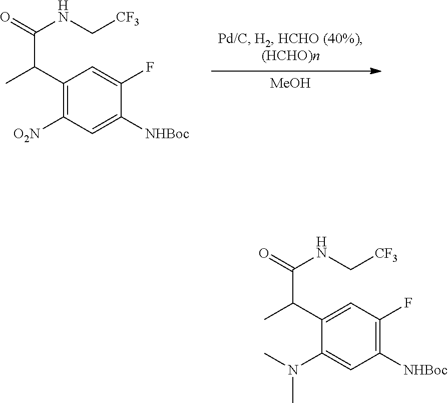 [SMILES: "MeOH" ; CC(C(=O)NCC(F)(F)F)c1cc(F)c(NC(=O)OC(C)(C)C)cc1[N+](=O)[O-] ; "Pd/C, H2, HCHO (40%),(HCHO)n" ; CC(C(=O)NCC(F)(F)F)c1cc(F)c(NC(=O)OC(C)(C)C)cc1N(C)C]

To a solution of tert-butyl (2-fluoro-5-nitro-4-(1-oxo-1-((2,2,2-trifluoroethyl)amino)propan-2-yl)phenyl)carbamate (200 mg, 488 umol, 1.00 eq) in MeOH (5.00 mL) was added HCHO (2.18 g, 26.8 mmol, 20.0 mL, 37.0% purity, 54.9 eq), (HCHO)n (50.0 mg, 488 umol, 1.00 eq), and Pd/C (20.0 mg, 10.0% purity). The reaction mixture was then stirred at RT for 10 h under H₂. The reaction mixture was filtered and concentrated under reduced pressure to afford tert-butyl (5-(dimethylamino)-2-fluoro-4-(1-oxo-1-((2,2,2-trifluoroethyl)amino)propan-2-yl)phenyl)carbamate (0W mg, crude) as a white solid. ¹H NMR (400 MHz, CDCl₃) δ 8.03 (d, J=8.0 Hz, 1H), 7.40-7.31 (m, 1H), 7.02 (d, J=16.0 Hz, 1H), 6.70 (s, 1H), 4.21-4.09 (m, 1H), 3.96-3.62 (m, 2H), 2.70 (s, 6H), 1.54 (s, 9H), 1.46 (<. =8 0 Hz, 3H)

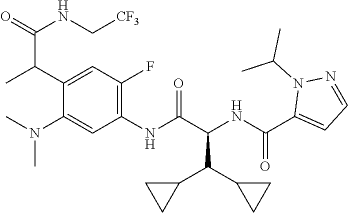 [SMILES: CC(C(=O)NCC(F)(F)F)c1cc(F)c(NC(=O)[C@@H](NC(=O)c2ccnn2C(C)C)C(C2CC2)C2CC2)cc1N(C)C]

N-((2S)-1,1-dicyclopropyl-3-((5-(dimethylamino)-2-fluoro-4-(1-oxo-1-((2,2,2-trifluoroethyl)amino)propan-2-yl)phenyl)amino)-3-oxopropan-2-yl)-1-isopropyl-1H-pyrazole-5-carboxamide. Prepared according to General Scheme G, employing (S)-2-((tert-butoxycarbonyl)amino)-3,3-dicyclopropylpropanoic acid and tert-butyl (5-(dimethylamino)-2-fluoro-4-(1-oxo-1-((2,2,2-trifluoroethyl)amino)propan-2-yl)phenyl)carbamate followed by 1-isopropyl-1H-pyrazole-5-carboxylic acid. The title compound was purified by chiral SFC to afford the first eluting, single stereoisomer (column: REGIS(S,S) WHELKO1 (250 mm * 25 mm, 10 um); mobile phase: |0.1% NH₃H₂O in IPA]; 45%). LCMS [M+H]⁺=595.3 m/z.

### Example 171: Synthesis of Compound 164

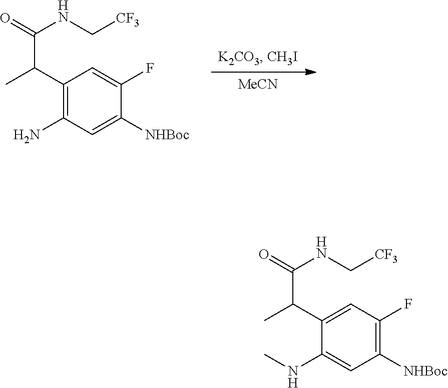

To a solution of tert-butyl (5-amino-2-fluoro-4-(1-oxo-1-((2,2,2-trifluoroethyl)amino)propan-2-yl)phenyl)carbamate (550 mg, 1.45 mmol, 1.00 eq) and K₂CO₃ (601 mg, 4.35 mmol, 3.00 eq) in MeCN (5.00 mL) was added Mel (411 mg, 2.90 mmol, 180 μL, 2.00 eq). The reaction mixture was stirred at 70° C. for 6 h. The reaction mixture was filtered and concentrated under reduced pressure to give a residue. The residue was purified by prep-TLC (SiO₂, pet-ether:ethyl acetate=2:1) to afford tert-butyl (2-fluoro-5-(methylamino)-4-(1-oxo-1-((2,2,2-trifluoroethyl)amino)propan-2-yl)phenyl)carbamate (80.0 mg, 203 umol, 14.0% yield) as a white solid.

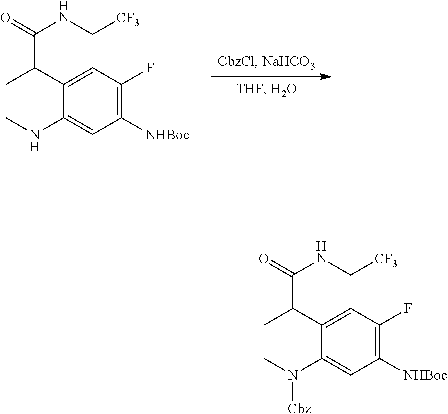

To a solution of tert-butyl (2-fluoro-5-(methylamino)-4-(1-oxo-1-((2,2,2-trifluoroethyl)amino)propan-2-yl)phenyl)carbamate (80.0 mg, 203 umol, 1.00 eq) and Na₂CO₃ (64.6 mg, 610 umol, 3.00 eq) in THF (5.00 mL) and H₂O (2.00 mL) was added CbzCI (69.3 mg, 406 umol, 57.8 uL, 2.00 eq) at 0° C. The reaction mixture was stirred at RT for 2 h. The reaction mixture was diluted with H₂O and extracted with EtOAc. The combined organic layers were dried over Na₂SO₄, filtered, and concentrated under reduced pressure to give a residue. The residue was purified by prep-TLC (SiO₂, Petroleum ether: Ethyl acetate=3: 1) to afford benzyl (5-((tert-butoxycarbonyl)amino)-4-fluoro-2-(1-oxo-1-((2,2,2-trifluoroethyl)amino)propan-2-yl)phenyl)(methyl)carbamate 100 m 189 umol 93.2% yield as a white solid.

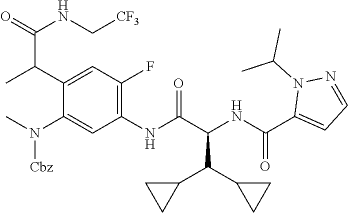 [SMILES: CC(C(=O)NCC(F)(F)F)c1cc(F)c(NC(=O)[C@@H](NC(=O)c2ccnn2C(C)C)C(C2CC2)C2CC2)cc1N(C)C(=O)OCc1ccccc1]

benzyl (5-((S)-3,3-dicyclopropyl-2-(1-isopropyl-1H-pyrazole-5-carboxamido)propanamido)-4-fluoro-2-(1-oxo-1-((2,2,2-trifluoroethyl)amino)propan-2-yl)phenyl)(methyl)carbamate. Prepared according to General Scheme G, employing (S)-2-((tert-butoxycarbonyl)amino)-3,3-dicyclopropylpropanoic acid and benzyl (5-((tert-butoxycarbonyl)amino)-4-fluoro-2-(1-oxo-1-((2,2,2-trifluoroethyl)amino)propan-2-yl)phenyl)(methyl)carbamate followed by 1-isopropyl-1H-pyrazole-5-carboxylic acid. The title compound was purified by chiral SFC to afford the second eluting, single stereoisomer (column: DAICEL CHIRALPAK AD (250 mm * 30 mm, 10 um): mobile phase: [0.1% NH₃H₂O in IPA]; 15%). ¹H NMR (400 MHz, CD₃0D) S 7.52 (d, J=2.0 Hz, 1H), 7.22 (d, J=6.8 Hz, 1H), 6.96 (d, J=11.6 Hz, 1H), 6.79 (d, J=2.0 Hz, 1H), 5.45-5.34 (m, 1H), 4.97 (d, .J=6.8 Hz, 1H), 3.92-3.66 (m, 3H), 3.31 (s, 3H), 2.79 (s, 3H), 1.50-1.37 (m, 9H), 0.94-0.76 (m, 3H), 0.59-0.23 (m, 8H).

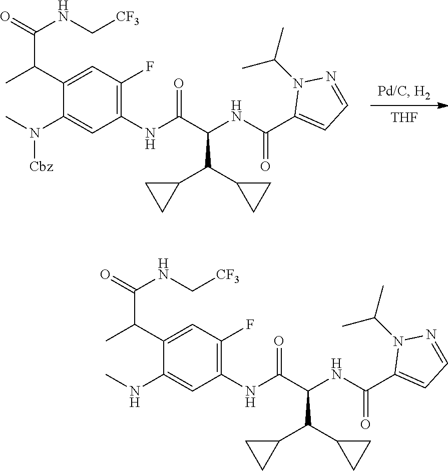 [SMILES: CC(C(=O)NCC(F)(F)F)c1cc(F)c(NC(=O)[C@@H](NC(=O)c2ccnn2C(C)C)C(C2CC2)C2CC2)cc1N(C)C(=O)OCc1ccccc1 ; "Pd/C, H2" ; "THF" ; CNc1cc(NC(=O)[C@@H](NC(=O)c2ccnn2C(C)C)C(C2CC2)C2CC2)c(F)cc1C(C)C(=O)NCC(F)(F)F]

To a solution of benzyl (5-((S)-3,3-dicyclopropyl-2-(1-isopropyl-1H-pyrazole-5-carboxamido)propanamido)-4-fluoro-2-(1-oxo-1-((2,2,2-trifluoroethyl)amino)propan- 2-yl)phenyl)(methyl)carbamate (30.0 mg, 41.9 umol, 1.00 eq) in THF (5.00 mL) was added Pd/C (3.0) mg, 10.0% purity) under N₂. The suspension was degassed and purged with H₂ 3 times. The reaction mixture was stirred under H₂ (15 Psi) at RT for 2 h. he reaction mixture was filtered, rinsing with MeOH, and then concentrated under reduced pressure to give a residue. The residue was purified by prep-HPLC (basic condition, column: Waters Xbridge BEH C18 150 * 25 mm * 5 um: mobile phase: [water (NH₃H₂O)-ACN]; B %: 36%-66%) to afford N-((2S)-1,1-dicyclopropyl-3-((2-fluoro-5-(methylamino)-4-(1-oxo-1-((2,2,2-trifluoroethyl)amino)propan-2-yl)phenyl)amino)-3-oxopropan-2-yl)-1-isopropyl-1H-pyrazole-5-carboxamide (13.31 mg, 22.1 umol, 53.6% yield, 96.7% purity) as a white solid. LCMS [M+H]J=581.5 m/z.

### Example 172: Synthesis of Compound 165

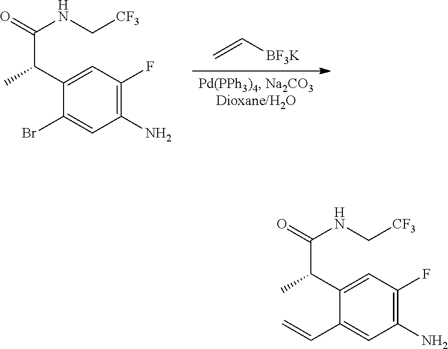

To a solution of (S)-2-(4-amino-2-bromo-5-fluorophenyl)-N-(2,2,2-trifluoroethyl)propanamide (1.50 g, 4.37 mmol, 1.00 eq), vinyl potassium trifluoroborate (1.17 g, 8.74 mmol, 2.00 eq) in dioxane (20.0 mL) and H₂O (2.00 mL) was added Pd(PPh₃)₄ (505 mg, 437 umol, 0.100 eq) and Na₂CO₃ (926 mg, 8.74 mmol, 2.00 eq). The reaction mixture was stirred at 80° C. for 24 h. The reaction mixture was diluted with H₂O, extracted with EtOAc, dried over anhydrous Na₂SO₄, filtrated, and concentrated under vacuum. The residue was purified by column chromatography (SiO₂, petroleum ether: EtOAc=20: 1 to 3: 1) to afford (S)-2-(4-amino-5-fluoro-2-vinylphenyl)-N-(2,2,2-trifluoroethyl)propanamide (600 mg, 2.07 mmol, 47.2% yield) as a yellow solid. LCMS [M+H]⁺=291.2 m/z.

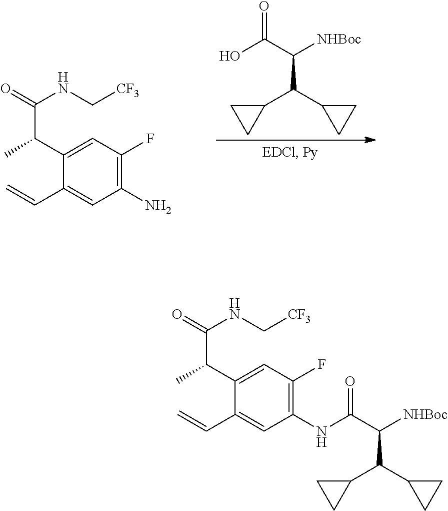 [SMILES: C=Cc1cc(N)c(F)cc1[C@H](C)C(=O)NCC(F)(F)F ; CC(C)(C)OC(=O)N[C@H](C(=O)O)C(C1CC1)C1CC1 ; C=Cc1cc(NC(=O)[C@@H](NC(=O)OC(C)(C)C)C(C2CC2)C2CC2)c(F)cc1[C@H](C)C(=O)NCC(F)(F)F]

tert-butyl ((S)-1,1-dicyclopropyl-3-((2-fluoro-4-((S)-1-oxo-1-((2,2,2-trifluoroethyl)amino)propan-2-yl)-5-vinylphenyl)amino)-3-oxopropan-2-yl)carbamate was prepared according to General Procedure D, employing (S)-2-(4-amino-5-fluoro-2-vinylphenyl)-N-(2,2,2-trifluoroethyl)propenamide and (S)-2-((tert-butoxycarbonyl)amino)-3,3-dicyclopropylpropanoic acid.

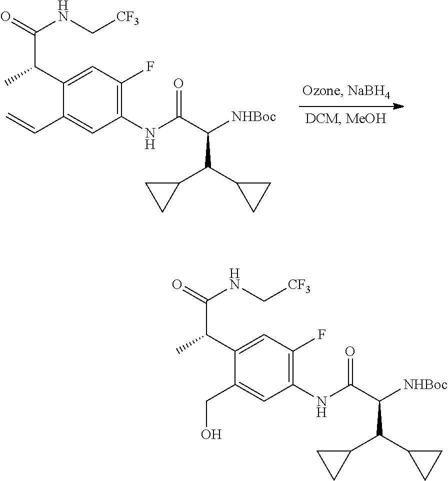 [SMILES: C=Cc1cc(NC(=O)[C@@H](NC(=O)OC(C)(C)C)C(C2CC2)C2CC2)c(F)cc1[C@H](C)C(=O)NCC(F)(F)F ; C[C@H](C(=O)NCC(F)(F)F)c1cc(F)c(NC(=O)[C@@H](NC(=O)OC(C)(C)C)C(C2CC2)C2CC2)cc1CO]

OZONE (15.0 Psi) was bubbled into a solution of tert-butyl ((S)-1,1-dicyclopropyl-3-((2 fluoro-4-((S)-1-oxo-1-4(2,2,2-trifluoroethyl)amino)propan-2-yl)-5-vinylphenyl)amino)-3-oxopropan-2-yl)carbamate (0.300 g, 553 umol, 1.00 eq) in DCM (20.0 mL) and MeOH (2.0) mL) at −70° C. for 0.5 h. Then, N₂ was bubbled to the reaction mixture at −70° C. for 0.5 h followed by the addition of NaBH₄ (168 mg, 4.44 mmol, 8.02 eq). The reaction mixture was warmed to RT and stirred for 2 h. The reaction mixture was treated with 3.0 mL of H₂O at 0° C. and then diluted with 50 mL of H₂O and extracted with DCM. The combined organic layers were dried over Na₂SO₄, filtered, and concentrated under reduced pressure to give a residue. The residue was purified by prep-TLC (SiO₂, petroleum ether: ethyl acetate=1: 1) to afford tert-butyl ((S)-1,1-dicyclopropyl-3-((2-fluoro-5-(hydroxymethyl)-4-((S)-1-oxo-1-((2,2,2-trifluoroethyl)amino)propan-2-yl)phenyl)amino)-3-oxopropan-2-yl)carbamate (150 mg, 274 umol, 49.6% yield) as a white solid.

LCMS [M+H]⁺=546.1 m/z.

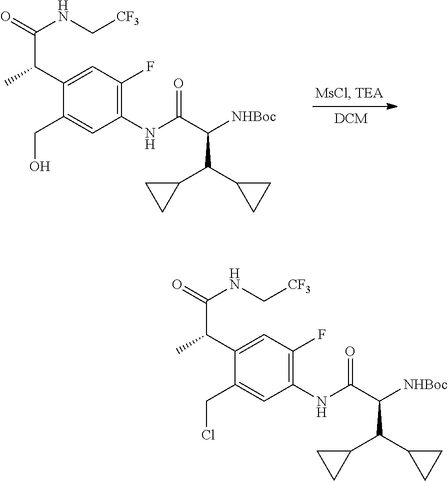 [SMILES: C[C@H](C(=O)NCC(F)(F)F)c1cc(F)c(NC(=O)[C@@H](NC(=O)OC(C)(C)C)C(C2CC2)C2CC2)cc1CO ; C[C@H](C(=O)NCC(F)(F)F)c1cc(F)c(NC(=O)[C@@H](NC(=O)OC(C)(C)C)C(C2CC2)C2CC2)cc1CCl]

To a solution of tert-butyl ((S)-1.1-dicyclopropyl-3-((2-fluoro-5-(hydroxymethyl)-4-((S)-1-oxo-1-((2,2,2-trifluoroethyl)amino)propan-2-yl)phenyl)amino)-3-oxopropan-2-yl)carbamate (100 mg, 178 umol, 1.00 eq) in DCM (2.00 mL) was added MsCl (40.9 mg, 357 umol, 27.6 uL, 2.00 eq) and TEA (45.2 mg, 446 umol, 62.1 uL, 2.50 eq) at 0° C. The reaction mixture was stirred at RT for 2 h. The reaction mixture was diluted with H₂O, extracted with EtOAc, dried over anhydrous Na₂SO₄, filtrated, and concentrated under vacuum to afford tert-butyl ((S)-1-((5-(chloromethyl)-2-fluoro-4-((S)-1-oxo-1-((2,2,2-trifluoroethyl)amino)propan-2-yl)phenyl)amino)-3,3-dicyclopropyl-1-oxopropan-2-yl)carbamate (100 mg, 177 umol, 99.2% yield) as a white solid. LCMS [M+H=464.2 m/z.

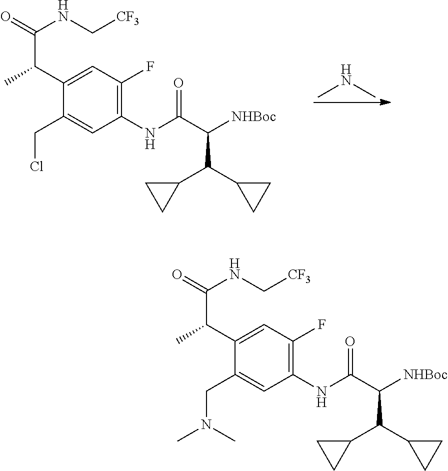

To a solution of tert-butyl ((S)-1-((5-(chloromethyl)-2-fluoro-4-((S)-1-oxo-1-((2,2,2-trifluoroethyl)amino)propan-2-yl)phenyl)amino)-3,3-dicyclopropyl-1-oxopropan-2-yl)carbamate (100 mg, 177 umol, 1.00 eq) was added dimethylamine (242 mg, 1.77 mmol, 272 μL, 33.0% purity, 10.0 eq). The reaction mixture was stirred at RT for 2 h. The reaction mixture was concentrated under vacuum. The residue was purified by prep-TLC (SiO₂, petroleum ether: ethyl acetate=3: 1) to afford tert-butyl ((S)-1,1-dicyclopropyl-3-((5-((dimethylamino)methyl)-2-fluoro-4-((S)-1-oxo-1-((2,2,2-trifluoroethyl)amino)propan-2-yl)phenyl)amino)-3-oxopropan-2-yl)carbamate (90.0 mg, 157 umol, 88.6% yield) as a white solid. LCMS [M+H]⁺=573.3 m/z.

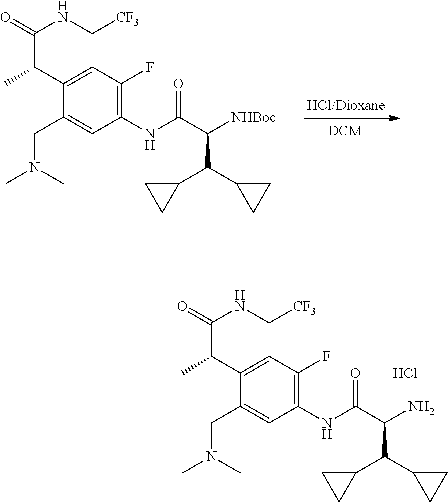 [SMILES: C[C@H](C(=O)NCC(F)(F)F)c1cc(F)c(NC(=O)[C@@H](NC(=O)OC(C)(C)C)C(C2CC2)C2CC2)cc1CN(C)C ; C[C@H](C(=O)NCC(F)(F)F)c1cc(F)c(NC(=O)[C@@H](N)C(C2CC2)C2CC2)cc1CN(C)C ; Cl]

(S)-2-amino-3,3-dicyclopropyl-N-(5-((dimethylamino)methyl)-2-fluoro-4-((S)-1-oxo-1-((2,2,2-trifluoroethyl)amino)propan-2-yl)phenyl)propenamide was prepared according to General Procedure C, employing tert-butyl ((S)-1,1-dicyclopropyl-3-((5-((dimethylamino)methyl)-2-fluoro-4-((S)-1-oxo-1-((2,2,2-trifluoroethyl)amino)propan-2-yl)phenyl)amino)-3-oxopropan-2-yl)carbamate.

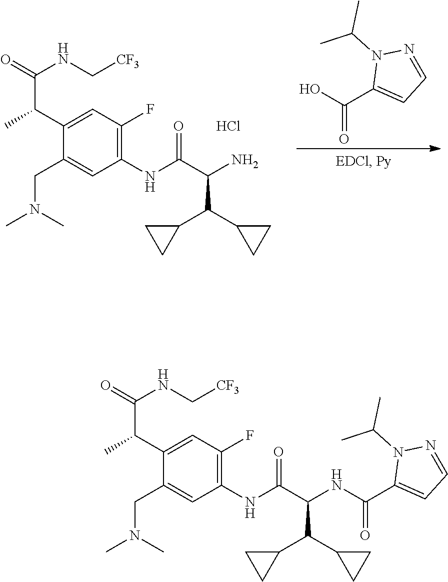 [SMILES: C[C@H](C(=O)NCC(F)(F)F)c1cc(F)c(NC(=O)[C@@H](N)C(C2CC2)C2CC2)cc1CN(C)C ; Cl ; CC(C)n1nccc1C(=O)O ; CC(C)n1nccc1C(=O)N[C@H](C(=O)Nc1cc(CN(C)C)c([C@H](C)C(=O)NCC(F)(F)F)cc1F)C(C1CC1)C1CC1]

N-((S)-1,1-dicyclopropyl-3-((5-((dimethylamino)methyl)-2-fluoro-4-((S)-1-oxo-1-((2,2,2-trifluoroethyl)amino)propan-2-yl)phenyl)amino)-3-oxopropan-2-yl)-1-isopropyl-1H-pyrazole-5-carboxamide was prepared according to General Procedure D. employing (S)-2-amino-3,3-dicyclopropyl-N-(5-((dimethylamino)methyl)-2-fluoro-4-((S)-1-oxo-1-((2,2,2-trifluoroethyl)amino)propan-2-yl)phenyl)propenamide and 1-isopropyl-1H-pyrazole-5-carboxylic acid. LCMS [M+H]⁺=609.4 m/z.

### Example 173: Synthesis of Compound 166

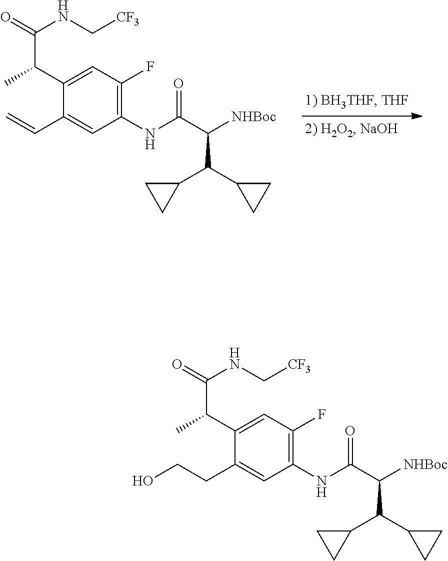 [SMILES: C=Cc1cc(NC(=O)[C@@H](NC(=O)OC(C)(C)C)C(C2CC2)C2CC2)c(F)cc1[C@H](C)C(=O)NCC(F)(F)F ; C[C@H](C(=O)NCC(F)(F)F)c1cc(F)c(NC(=O)[C@@H](NC(=O)OC(C)(C)C)C(C2CC2)C2CC2)cc1CCO]

To a solution of tert-butyl ((S)-1,1-dicyclopropyl-3-((2-fluoro-4-((S)-1-oxo-1-((2,2,2-trifluoroethyl)amino)propan-2-yl)-5-vinylphenyl)amino)-3-oxopropan-2-yl)carbamate (0.4 g, 738 umol, 1.00 eq) in THF (5.00 mL) at RT was added BH₃THF (1 M, 3.18 mL, 4.30 eq). dropwise. The reaction mixture was stirred for 2 h before adding 2.0 mL of H₂O and NaOH (196 mg, 1.48 mmol, 30.0% purity, 2.00 eq) to reach pH 12. H₂O₂ (739 mg, 6.52 mmol, 626 μL, 30.0% purity, 8.83 eq) was added, and the reaction mixture was heated at 80° C. for 1 h. The reaction mixture was poured over sat. Na₂SO₃ (20.0 mL) and extracted with ethyl acetate. The combined organic layers were washed with brine, dried over anhydrous Na₂SO₄, filtered, and concentrated under reduced pressure to give a residue. The residue was purified by prep-HPLC (neutral condition; column: Welch Ultimate XB—CN 250 * 50 * 10 um; mobile phase: [Hexane-EtOH (0.1% NH₃.H₂O)]; B %: 1%-40%) followed by prep-HPLC (neutral condition; column: Waters Xbridge 150 * 25 mm * 5 um; mobile phase: [water (ammonia hydroxide v/v)-ACN]; B %: 38% -68%) to afford tert-butyl ((S)-1,1-dicyclopropyl-3-((2-fluoro-5-(2-hydroxyethyl)-4-((S)-1-oxo-1-((2,2,2-trifluoroethyl)amino)propan-2-yl)phenyl)amino)-3-oxopropan-2-yl)carbamate (30.0 mg, 53.6 umol, 7.26% yield) as a white solid.

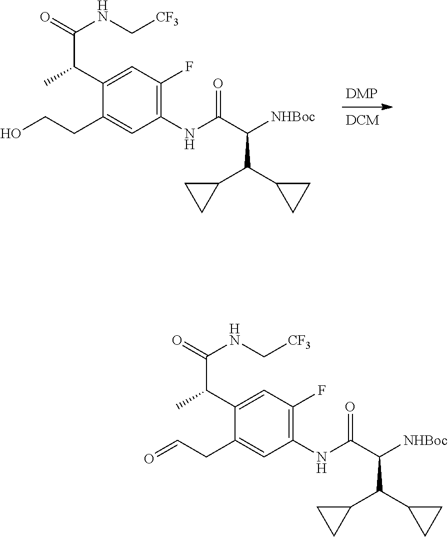 [SMILES: C[C@H](C(=O)NCC(F)(F)F)c1cc(F)c(NC(=O)[C@@H](NC(=O)OC(C)(C)C)C(C2CC2)C2CC2)cc1CCO ; C[C@H](C(=O)NCC(F)(F)F)c1cc(F)c(NC(=O)[C@@H](NC(=O)OC(C)(C)C)C(C2CC2)C2CC2)cc1CC=O]

To a solution of tert-butyl ((S)-1,1-dicyclopropyl-3-((2-fluoro-5-(2-hydroxyethyl)-4-((S)-1-oxo-1-((2,2,2-trifluoroethyl)amino)propan-2-yl)phenyl)amino)-3-oxopropan-2-yl)carbamate (30.0 mg, 53.6 umol, 1.00 eq) in DCM (2.00 mL) at 0° C. was added DMP (34.1 mg, 64.3 umol, 24.9 uL, 80.0% purity, 1.20 eq). The reaction mixture was stirred for 1 h at RT. The reaction mixture was poured over sat. aq. NaHCO₃ and extracted with EtOAc. The combined organic layers were washed with Na₂SO₃. dried over Na₂SO₄, filtered, and concentrated under reduced pressure to afford tert-butyl ((S)-1,1-dicyclopropyl-3-((2-fluoro-4-((S)-1-oxo-1-((2,2,2-trifluoroethyl)amino)propan-2-yl)-5-(2-oxoethyl)phenyl)amino)-3-oxopropan-2-yl)carbamate (25.0 mg, 44.8 umol, 83.6% yield) as a yellow solid.

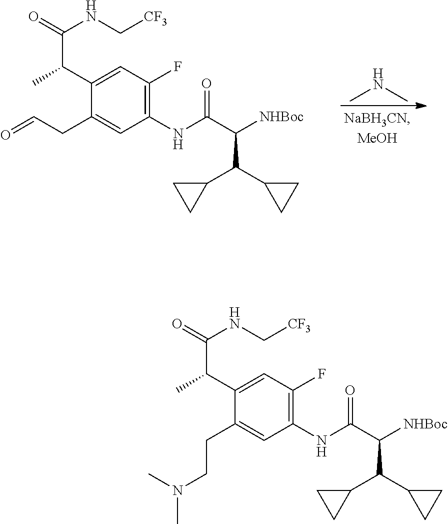

To a solution of tert-buty1 ((S)-1,1-dicyclopropyl-3-((2-fluoro-4-((S)-1-oxo-1-((2,2,2-trifluoroethyl)amino)propan-2-yl)-5-(2-oxoethyl)phenyl)amino)-3-oxopropan-2-yl)carbamate (25.0 mg, 44.8 umol, 1.00 eq) in MeOH (2.00 mL) was added dimethylanne (12.2 mg, 89.6 umol, 13.7 uL, 33.0% purity, 2.00 eq) and NaBH₃CN (5.64 mg, 89.6 umol, 2.00 eq). The reaction mixture was stirred at RT for 1 h. The reaction mixture was diluted with H₂O and extracted with EtOAc, dried with anhydrous Na₂SO₄, filtrated, and concentrated under vacuum. The residue was purified by prep-HPLC (neutral condition: column: Waters Xbridge 150 * 25 mm * 5 um; mobile phase: [water (ammonia hydroxide v/v)-ACN]; 46%-76%) to afford tert-butyl ((S)-1,1-dicyclopropyl-3-((5-(2-(dimethylamino)ethyl)-2-fluoro-4-((S)-1-oxo-1-((2,2,2-trifluoroethyl)amino)propan-2-yl)phenyl)amino)-3-oxopropan-2-yl)carbamate (8.00 mg, 13.6 umol, 30.4% yield) as a white solid.

LCMS [M+H]⁺=587.4 m/z.

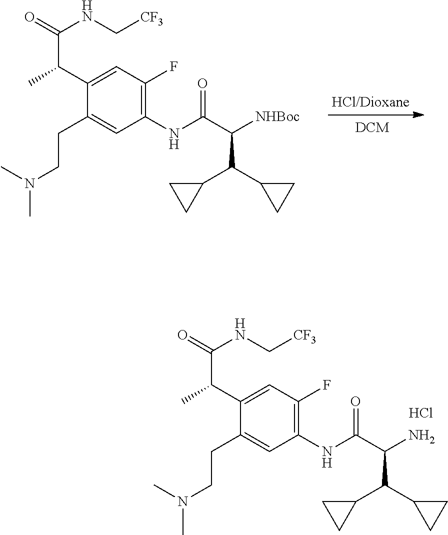 [SMILES: C[C@H](C(=O)NCC(F)(F)F)c1cc(F)c(NC(=O)[C@@H](NC(=O)OC(C)(C)C)C(C2CC2)C2CC2)cc1CCN(C)C ; C[C@H](C(=O)NCC(F)(F)F)c1cc(F)c(NC(=O)[C@@H](N)C(C2CC2)C2CC2)cc1CCN(C)C ; Cl]

(S)-2-amino-3,3-dicyclopropyl-N-(5-(2-(dimethylamino)ethyl)-2-fluoro-4-((S)-1-oxo-1-((2,2,2-trifluoroethyl)amino)propan-2-yl)phenyl)propenamidehydrochloride was prepared according to General Procedure C, employing tert-butyl ((S)-1,1-dicyclopropyl-3-((5-(2-(dimethylamino)ethyl)-2-fluoro-4-((S)-1-oxo-1-((2,2,2-trifluoroethyl)amino)propan-2-yl)phenyl)amino)-3-oxopropan-2-yl)carbamate.

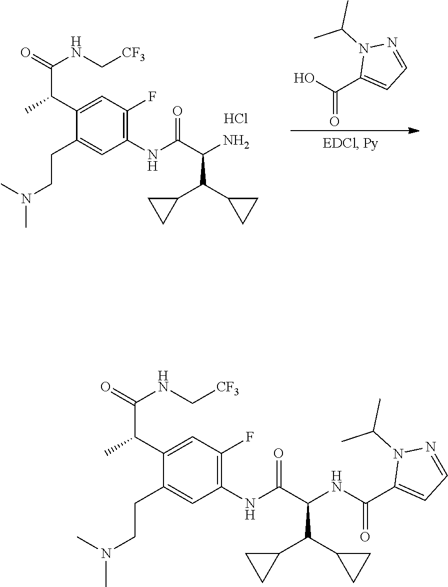 [SMILES: C[C@H](C(=O)NCC(F)(F)F)c1cc(F)c(NC(=O)[C@@H](N)C(C2CC2)C2CC2)cc1CCN(C)C ; Cl ; CC(C)n1nccc1C(=O)O ; CC(C)n1nccc1C(=O)N[C@H](C(=O)Nc1cc(CCN(C)C)c([C@H](C)C(=O)NCC(F)(F)F)cc1F)C(C1CC1)C1CC1]

N-((S)-1,1-dicyclopropyl-3-((5-(2-(dimethylamino)ethyl)-2-fluoro-4-((S)-1-oxo-1-((2,2,2-trifluoroethyl)amino)propan-2-yl)phenyl)amino)-3-oxopropan-2-yl)-1-isopropyl-1H-pyrazole-5-carboxamide was prepared according to General Procedure D, employing (S)-2-amino-3,3-dicyclopropyl-N-(5-(2-(dimethylamino)ethyl)-2-fluoro-4-((S)-1-oxo-1-((2,2,2-trifluoroethyl)amino)propan-2-yl)phenyl)propenamide hydrochloride and 1-isopropyl-1H-pyrazole-5-carboxylic acid. LCMS [M+H]⁺=609.4 m/z.

### Example 174: Synthesis of Compound 167

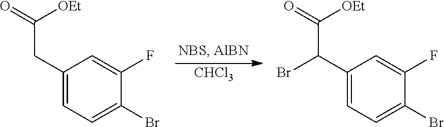

To a solution of ethyl 2-(4-bromo-3-fluorophenyl)acetate (2.00 g, 7.66 mmol, 1.00 eq) in CHCl₃ (5.00 mL) was added NBS (1.64 g, 9.19 mmol, 1.20 eq) and AIBN (125 mg, 766 umol, 0.100 eq). The reaction mixture was stirred at 60° C. for 1 h. The reaction mixture was diluted with H₂O and extracted with EtOAc. The combined organic layers were washed with brine, dried over Na₂SO₄, filtered, and concentrated under reduced pressure to give a residue. The residue was purified by column chromatography (SiO₂, pet-ether/EtOAc=100:1 to 10:1) to afford ethyl 2-bromo-2-(4-bromo-3-fluorophenyl)acetate (1.10 g, 3.24 mmol, 42.2% yield) as a white solid. ¹H NMR (400 MHz, CDCl₃) δ 7.59-7.52 (m, 1H), 7.39 (dd, J₁=2.0, J₂=9.0 Hz, 1H), 7.20 (dd, J₁=1.6, J₂=8.0 Hz, 1H), 5.26 (s, 1H), 4.31-4.21 (m, 2H), 1.30 (t, J=7.2 Hz, 3H).

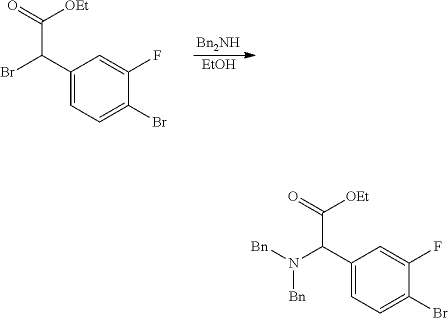 [SMILES: "Bn2NH" ; "EtOH" ; CCOC(=O)C(Br)c1ccc(Br)c(F)c1 ; CCOC(=O)C(c1ccc(Br)c(F)c1)N(Cc1ccccc1)Cc1ccccc1]

To a solution of ethyl 2-bromo-2-(4-bromo-3-fluorophenyl)acetate (1.00 g, 2.94 mmol, 1.00 eq) in EtOH (10.0 mL) was added dibenzylamine (1.16 g, 5.88 mmol, 1.13 mL, 2.00 eq). The reaction mixture was stirred at 80° C. for 8 h. The reaction mixture was diluted with H₂O and extracted with EtOAc. The combined organic layers were dried over Na₂SO₄, filtered, and concentrated under reduced pressure to give a residue. The residue was purified by column chromatography (SiO₂, Petroleum ether/Ethyl acetate=100/1 to 10/1) to afford ethyl 2-(4-bromo-3-fluorophenyl)-2-(dibenzylamino)acetate (1.00 g, 2.19 mmol, 74.5% yield) as a white solid. ¹H NMR (400 MHz, CDCl₃)_{5 7.56} (t, J=7.2, 1H), 7.43-7.34 (m, 8H), 7.32-7.24 (m, 3H), 7.10 (dd. J₁=1.6, J₂=8.2 Hz, 1H), 4.58 (s, 1H), 4.35 (m, 2H), 3.86-3.72 (m, 4H), 1.38 (t, J=7.2 Hz, 3H).

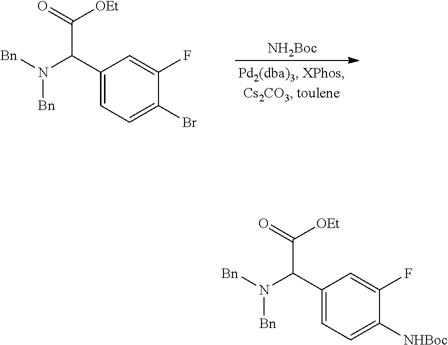 [SMILES: CCOC(=O)C(c1ccc(Br)c(F)c1)N(Cc1ccccc1)Cc1ccccc1 ; CCOC(=O)C(c1ccc(NC(=O)OC(C)(C)C)c(F)c1)N(Cc1ccccc1)Cc1ccccc1 ; "NH2Boc"]

To a solution of ethyl 2-(4-bromo-3-fluorophenyl)-2-(dibenzylamino)acetate (1.00 g, 2.19 mmol, 1.00 eq), tert-butyl carbamate (308 mg, 2.63 mmol, 1.20 eq) in toluene (5.00 mL) was added XPhos (104 mg, 219 umol, 0.100 eq), Cs2CO₃ (1.43 g, 4.38 mmol, 2.00 eq), and Pd₂(dba)₃ (200 mg, 219 umol, 0.100 eq). The reaction mixture was stirred at 80° C. for 2 h. The reaction mixture was diluted with H₂O and extracted with EtOAc. The combined organic layers were dried over Na₂SO₄, filtered, and concentrated under reduced pressure to give a residue. The residue was purified by column chromatography (SiO₂, Petroleum ether/Ethyl acetate=I00/1 to 10/1) followed by prep-HPLC (column: Welch Ultimate XB−CN 250 * 70 * 10 um; mobile phase: [Heptane -EtOH (0.1% NH₃H₂O)]; 1%-25%) to afford ethyl 2-(4-((tert-butoxycarbonyl)amino)-3-fluorophenyl)-2-(dibenzylamino)acetate (0.90 g, 1.83 mmol, 83.3% yield) as a white solid. ¹H NMR (400 MHz, CDCl₃) δ 8.04-7.97 (m, 1H), 7.30-7.23 (m, 8H), 7.21-7.15 (m, 2H), 7.10-6.99 (m, 2H), 6.64 (s, 1H), 4.45 (s, 1H), 4.28-4.13 (m, 2H), 3.76-3.60 (m, 4H), 1.46 (s, 9H), 1.25 (t. =7.2 Hz, 3H).

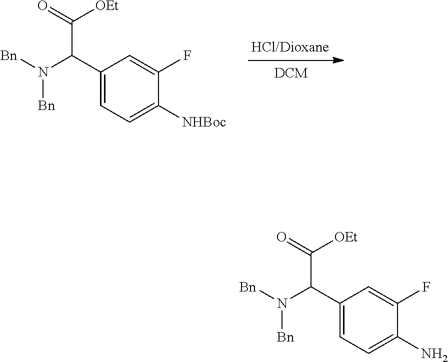 [SMILES: CCOC(=O)C(c1ccc(NC(=O)OC(C)(C)C)c(F)c1)N(Cc1ccccc1)Cc1ccccc1 ; CCOC(=O)C(c1ccc(N)c(F)c1)N(Cc1ccccc1)Cc1ccccc1]

ethyl 2-(4-amino-3-fluorophenyl)-2-(dibenzylamino)acetate was prepared according to General Procedure C. employing ethyl 2-(4-((tert-butoxycarbonyl)amino)-3-fluorophenyl)-2-(dibenzylamino)acetate.

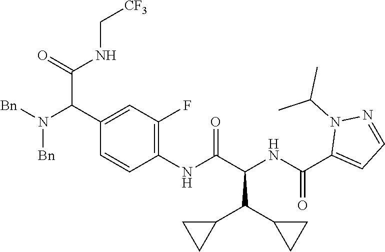 [SMILES: CC(C)n1nccc1C(=O)N[C@H](C(=O)Nc1ccc(C(C(=O)NCC(F)(F)F)N(Cc2ccccc2)Cc2ccccc2)cc1F)C(C1CC1)C1CC1]

N-((2S)-1,1-dicyclopropyl-3-((4-(1-(dibenzylamino)-2-oxo-2-((2,2,2-trifluoroethyl)amino)ethyl)-2-fluorophenyl)amino)-3-oxopropan-2-yl)-1-isopropyl-1H-pyrazole-5-carboxamide. Prepared according to General Scheme C, employing ethyl 2-(4-amino-3-fluorophenyl)-2-(dibenzylamino)acetate and (S)-2-((tert-butoxycarbonyl)amino)-3,3-dicyclopropylpropanoic acid followed by 1-isopropyl-1H-pyrazole-5-carboxylic acid and 2,2,2-trifluoroethan-1-amine. LCMS [M+H]⁺=733.5 m/z.

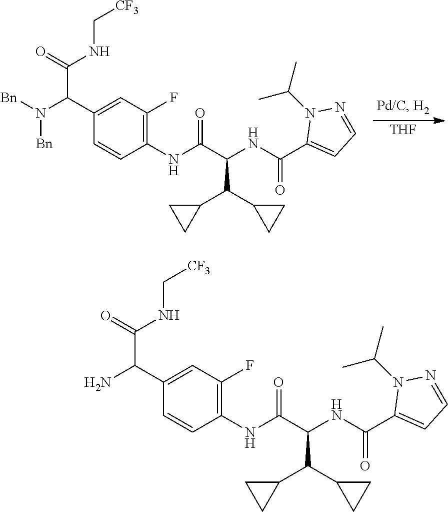

To a solution of N-((2S)-1,1-dicyclopropyl-3-((⁴-(1-(dibenzylamino)-2-oxo-2-((2,2,2-trifluoroethyl)amino)ethyl)-2-fluorophenyl)amino)-3-oxopropan-2-yl)-1-isopropyl-1H-pyrazole-5-carboxamide (400 mg, 545 umol, 1.00 eq) in DCM (5.00 mL) was added Pd/C (40.0 mg, 10.0% purity) under N₂. The suspension was degassed and purged with H₂ three times. The reaction mixture was stirred under H₂ (15 Psi) at RT for 2 h. The reaction mixture was filtered, rinsing with 100 mL of MeOH, and the filtrate was concentrated under reduced pressure to afford N-((2S)-1 -((4-(1-amino-2-oxo-2-((2,2,2-trifluoroethyl)amino)ethyl)-2-fluorophenyl)amino)-3,3-dicyclopropyl-1-oxopropan-2-yl)-1-isopropyl-1H-pyrazole-5-carboxamide (250 mg, 452 umol, 82.8% yield) as a white solid. LCMS [M+H]⁺=553.4 m/z.

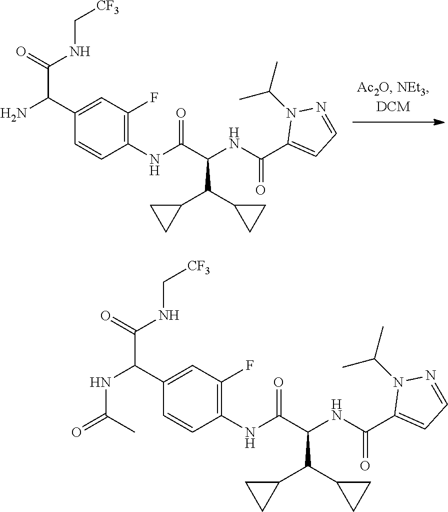

To a solution of N-((2S)-1-((4-(1-amino-2-oxo-2-((2,2,2-trifluoroethyl)amino)ethyl)-2-fluorophenyl)amino)-3,3-dicyclopropyl-1-oxopropan-2-yl)-1-isopropyl-1H-pyrazole-5-carboxamide (200 mg, 361 umol, 1.00 eq) in DCM (5.00 mL) was added Et₃N (109 mg, 1.09 mmol, 151 μL, 3.00 eq) and acetyl acetate (55.4 mg, 542 umol, 50.8 uL, 1.50 eq) at 0° C. The reaction mixture was stirred at RT for 0.5 h. The reaction mixture was diluted with H₂O and extracted with EtOAc. The combined organic layers were washed with sat. aq. NaHCO₃. dried over Na₂SO₄, filtered, and concentrated under reduced pressure to give a residue. The residue was purified by prep-HPLC (FA condition, column: Phenomenex C18 75 * 30 mm * 3 um: mobile phase: [water (FA)−ACN]; B %: 35%-65%). The title compound was isolated as the second eluting, single stereoisomer by chiral SFC (Daicel ChiralPak IG (250 * 30 mm, 10 um), eluting with 30% 0.1% NH₃H₂O in MEOH) (23.2 mg, 36.5 umol, 10.0% yield, white solid). LCMS [M+H]=595.4 m/z.

### Example 175: Synthesis of Comound 168

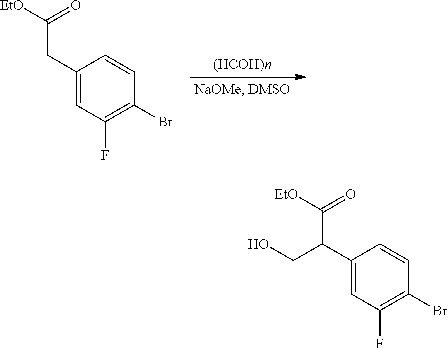 [SMILES: CCOC(=O)Cc1ccc(Br)c(F)c1 ; CCOC(=O)C(CO)c1ccc(Br)c(F)c1]

To a solution of ethyl 2-(4-bromo-3-fluorophenyl)acetate (4.00 g, 15.3 mmol, 1.00 eq) in DMSO (20.0 mL) was added HCHO (506 mg, 16.8 mmol, 464 μL, 1.10 eq) and NaOMe (41.3 mg, 766 umol, 0.0500 eq). The reaction mixture was stirred at RT for 12 h. The reaction solution was diluted with water and extracted with EtOAc. The combined organic layers were washed with water and brine, dried over Na₂SO₄, and concentrated under reduced pressure to give a residue. The residue was purified by column chromatography (SiO₂, petroleum ether:EtOAc=100:0 to 3:1) to afford ethyl 2-(4-bromo-3-fluorophenyl)-3-hydroxypropanoate (2.16 g, 7.42 mmol, 48.4% yield) as a colorless oil. ¹H NMR (400 MHz, DMSO) δ 7.66 (t, J=8.0 Hz, 1H), 7.34 (dd, J₁=2.0 Hz, J₂=2.0 Hz, 1H), 7.12 (dd, J₁=2.0 Hz, J₂=2.0 Hz, 1H), 5.07 (t, J=5.07 Hz, 1H), 4.12-4.05 (m, 2H), 3.89-3.80 (m, 2H), 3.68-3.63 (m, 1H), 1.15 (t, J=7.2 Hz, 3H).

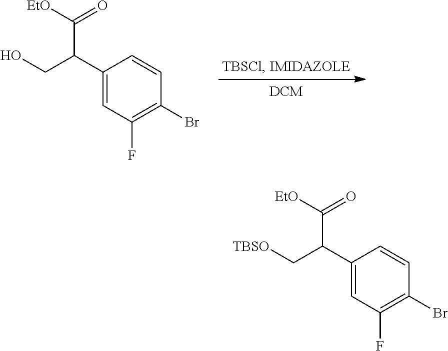 [SMILES: CCOC(=O)C(CO)c1ccc(Br)c(F)c1 ; CCOC(=O)C(CO[Si](C)(C)C(C)(C)C)c1ccc(Br)c(F)c1]

To a solution of ethyl 2-(4-bromo-3-fluorophenyl)-3-hydroxypropanoate (2.16 g, 7.42 mmol, 1.00 eq) in DCM (10.0 mL) was added TBSCI (2.24 g, 14.8 mmol, 1.82 mL, 2.00 eq) and imidazole (1.26 g, 18.5 mmol, 2.50 eq). The reaction mixture was stirred at RT for 2 h. The reaction solution was diluted with water and extracted with DCM. The combined organic layers were washed with water and brine, dried over Na₂SO₄, and concentrated under reduced pressure to give a residue. The residue was purified by prep-HPLC (column: Welch Ultimate XB−CN 250 * 70 * 10 um; mobile phase: [Hexane−EtOH]: B %: 1%-20%, 20 min) to afford ethyl 2-(4-bromo-3-fluorophenyl)-3-((tert-butyldimethylsilyl)oxy)propanoate (2.50 g, 6.17 mmol, 83.1% yield) as a colorless oil. ¹H NMR (400 MHz, CDCl₃) h 7.49 (t. =8.0 Hz, 1H), 7.16 (dd,J₁=2.0 Hz, J₂=1.6 Hz, 1H), 7.01 (dd, J₁=1.6 Hz,12=2.0 Hz, 1H), 4.23-4.07 (m, 3H), 3.83-3.73 (m, 2H), 1.25 (t, J=7.2 Hz, 3H), 0.85 (s, 9H), 0.01 (d, J=7.6 Hz, 6H).

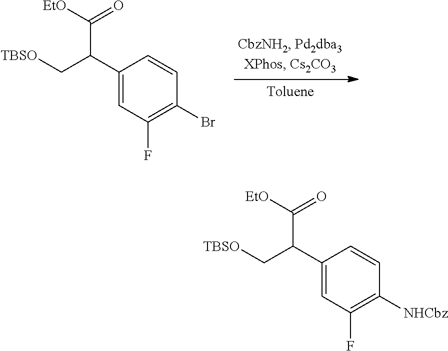 [SMILES: CCOC(=O)C(CO[Si](C)(C)C(C)(C)C)c1ccc(Br)c(F)c1 ; CCOC(=O)C(CO[Si](C)(C)C(C)(C)C)c1ccc(NC(=O)OCc2ccccc2)c(F)c1]

To a solution of ethyl 2-(4-bromo-3-fluorophenyl)-3-((tert- PGP495,C₃ butyldimethylsilyl)oxy)propanoate (1.80 g, 4.44 mmol, 1.00 eq) in toluene (50.0 mL) was added CbzNH₂ (1.34 g, 8.88 mmol, 2.00 eq), Pd₂(dba)₃ (360 mg, 393 umol, 8.85¹¹ eq), XPhos (211 mg, 444 umol, 0.100 eq), and Cs₂CO₃ (2.89 g, 8.88 mmol, 2.00 eq) at RT. The reaction mixture was stirred at 100° C. for 3 h. The solution was diluted with water and extracted with ethyl acetate. The combined organic layers were washed with water and brine, then dried over Na₂SO₄ and concentrated under reduced pressure to give a residue. The residue was purified by column chromatography (SiO₂, petroleum ether: ethyl acetate=100:0 to 4:1, petroleum ether: ethyl acetate =20:1) to afford ethyl 2-(4-(((benzyloxy)carbonyl)amino)-3-fluorophenyl)-3-((tert-butyldimethylsilyl)oxy)propanoate (2.00 g, 4.21 mmol, 94.70% yield) as a green solid. ¹H NMR (400 MHz, CDCl₃) δ 7.64-7.63 (m, 1H), 7.44-7.40 (m, 5H), 7.14-7.13 (m, 1H), 7.09 (s, 1H), 6.91 (s, 1H), 5.22 (s, 2H), 4.22-4.07 (m, 3H), 3.80-3.71 (m, 2H), 1.25 (t, J=7.2 Hz, 3H), 0.86 (s, 9H), 0.02 (d, J=6.0 Hz, 6H).

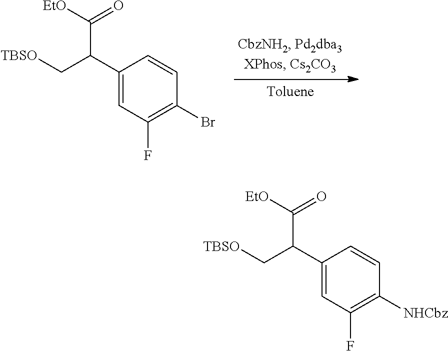 [SMILES: CCOC(=O)C(CO[Si](C)(C)C(C)(C)C)c1ccc(Br)c(F)c1 ; CCOC(=O)C(CO[Si](C)(C)C(C)(C)C)c1ccc(NC(=O)OCc2ccccc2)c(F)c1]

N-((2S)-1,1-dicyclopropyl-3-((4-(1-(dibenzylamino)-2-oxo-2-((2,2,2-trifluoroethyl)amino)ethyl)-2-fluorophenyl)amino)-3-oxopropan-2-yl)-1-isopropyl-1H-pyrazole-5-carboxamide. Prepared according to General Scheme C, employing ethyl 2-(4-amino-3-fluorophenyl)-24dibenzylamino)acetate and (S)-2-((tert-butoxycarbonyl)amino)-3,3-dicyclopropylpropanoic acid followed by 1-isopropyl-1H-pyrazole-5-carboxylic acid and 2,2,2-trifluoroethan-1-amine. LCMS [M+H]⁺=733.5 m/z.

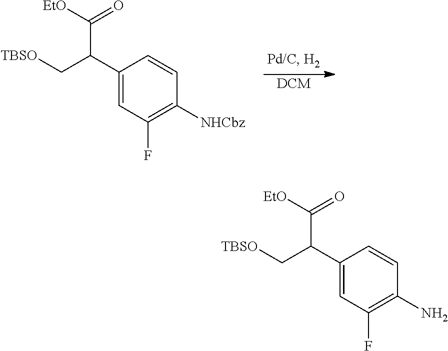

To a solution of ethyl 2-(4-(((benzyloxy)carbonyl)amino)-3-fluorophenyl)-3-((tert-butyldimethylsilyl)oxy)propanoate (2.00 g, 4.21 mmol, 1.00 eq) in DCM (30.0 mL) was added Pd/C (200 mg, 10.0% purity). The reaction mixture was degassed and purged with H₂ (15 psi) three times and stirred at RT for 3 h. The reaction mixture was filtered, and the filtrate was concentrated under reduced pressure to give a residue, which was purified by column chromatography (SiO₂, petroleum ether: ethyl acetate=100:0 to 4:1) to afford ethyl 2-(4-amino-3-fluorophenyl)-3-((tert-butyldimethylsilyl)oxy)propanoate (400 mg, 1.17 mmol, 27.86% yield) as a red oil.

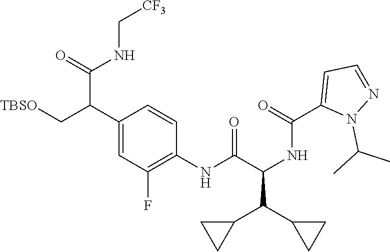 [SMILES: CC(C)n1nccc1C(=O)N[C@H](C(=O)Nc1ccc(C(CO[Si](C)(C)C(C)(C)C)C(=O)NCC(F)(F)F)cc1F)C(C1CC1)C1CC1]

trifluoroethyl)amino)propan-2-yl)-2-fluorophenyl)amino)-3,3-dicyclopropyl-1-oxopropan-2-yl)-1-isopropyl-1H-pyrazole-5-carboxamide. Prepared according to General Scheme C, employing ethyl 2-(4-amino-3-fluorophenyl)-3-((tert-butyldimethylsilyl)oxy)propanoate and (S)-2-(((benzyloxy)carbonyl)amino)-3,3-dicyclopropylpropanoic acid followed by 1-isopropyl-1H-pyrazole-5-carboxylic acid. Ester hydrolysis was conducted as described below, and the anide coupling was completed with 2,2,2-trifluoroethan-1-amine. LCMS [M+H]⁺=

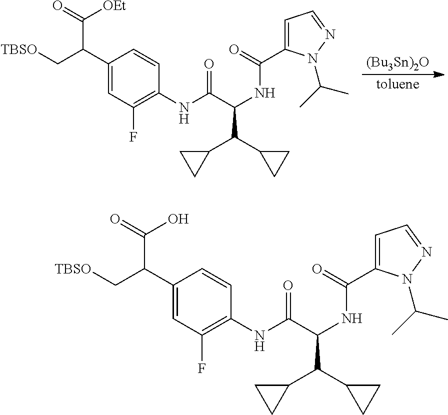

To a solution of ethyl 3-((tert-butyldimethylsilyl)oxy)-2-(4-((S)-3,3-dicyclopropyl-2-(1-isopropyl-1H-pyrazole-5-carboxamido)propanamido)-3-fluorophenyl)propanoate (200 mg, 318 umol, 1.00 eq) in toluene (10.0 mL) was added (Bu₃Sn)₂O (758 mg, 1.27 mmol, 648 μL, 4.00 eq) at 20° C. The reaction mixture was stirred at 100° C. for 48 h. The solution was quenched with sat. aq.

KF (20.0 mL) and stirred for 0.5 h. The solution was extracted with EtOAc, and the combined organic layers were washed with water and brine. The solution was then dried over Na₂SO₄ and concentrated under reduced pressure to give a residue. The residue was purified by column chromatography (SiO₂, petroleum ether: ethyl acetate=1:0 to 1:1) to afford 3-((tert-butyldimethylsilyl)oxy)-2-(4-((S)-3,3-dicyclopropyl-2-(1-isopropyl-1H-pyrazole-5-carboxamido)propanamido)-3-fluorophenyl)propanoic acid (100 mg, 166 umol, 52.3% yield) as a colorless oil. (400 MHz, CDCl₄). ¹H NMR 6 8.23-7.99 (m, 2H), 7.53 (s, 1H), 7.18-7.09 (m, 3H), 6.58 (s, 1H), 5.51-5.44 (m, 1H), 4.86-4.82 (m, 2H), 4.14-4.06 (m, 1H), 3.80-3.72 (m, 2H), 1.50-1.47 (m, 3H), 1.39-1.36 (m, 3H), 0.87-0.77 (m, I 1H), 0.68-0.52 (m, 4H), 0.42-0.24 (m, 4H), 0.08-0.05 (m, 6H).

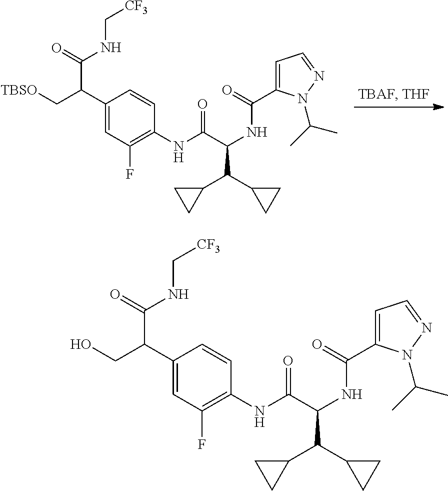

To a solution of N-((2S)-1 -((4-(3-((tert-butyldimethylsilyl)oxy)-1-oxo-1-((2,2,2-trifluoroethyl)amino)propan-2-yl)-2-fluorophenyl)amino)-3,3-dicyclopropyl-1- oxopropan-2-yl)-1-isopropyl-1H-pyrazole-5-carboxamide (70.0 mg, 103 umol, 1.00 eq) in THF (5.00 mL) was added TBAF (1.00 M, 205 μL, 2.00 eq) at 0° C., then stirred at RT for 1 h. The reaction solution was diluted with water and extracted with DCM. The combined organic layers were washed with water and brine, then dried over Na₂SO₄ and concentrated under reduced pressure to give a residue. The residue was purified by prep-TLC (DCM/MeOH=10:1) followed by chiral SFC (neutral condition: column: DAICEL CHIRALPAK AD (250 mm * 30 mm, 10 um), 30% MeOH) to afford N-((2S)-1.1-dicyclopropyl-3-((2-fluoro-4-(3-hydroay-1-oxo-11-((2,2,2-trifluoroethyl)amino)propan-2-yl)phenyl)amino)-3-oxopropan-2-yl)-1-isopropyl-1H-pyrazole-5-carboxamide (10.63 mg, 18.05 umol, 17.59% yield, 96.4% purity) as an off-white solid. LCMS [M+H]⁺=568.3 m/z.

### Example 176: Synthesis of Compounds 169-262

The compounds shown below in Table 1 were synthesized using analogous methods and procedures as described herein.

TABLE 1
Chemical Structures and Spectroscopic Data for Compounds 169-262

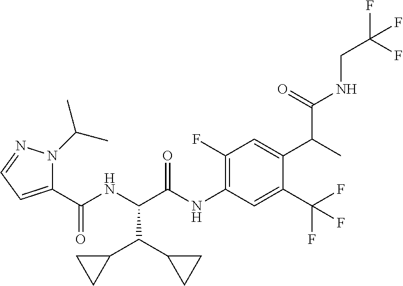 [SMILES: CC(C(=O)NCC(F)(F)F)c1cc(F)c(NC(=O)[C@@H](NC(=O)c2ccnn2C(C)C)C(C2CC2)C2CC2)cc1C(F)(F)F]

169 [M + H] = 620.3 m/z

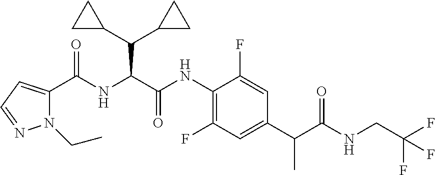 [SMILES: CCn1nccc1C(=O)N[C@H](C(=O)Nc1c(F)cc(C(C)C(=O)NCC(F)(F)F)cc1F)C(C1CC1)C1CC1]

170 [M + H] = 556.3 m/z
Single diastereomer with underdetermined
absolute stereochemistry

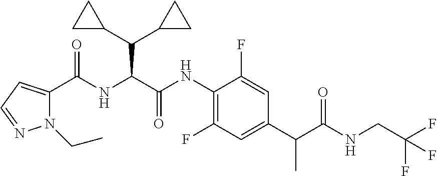 [SMILES: CCn1nccc1C(=O)N[C@H](C(=O)Nc1c(F)cc(C(C)C(=O)NCC(F)(F)F)cc1F)C(C1CC1)C1CC1]

171 [M + H] = 556.3 m/z
Single diastereomer with underdetermined
absolute stereochemistry

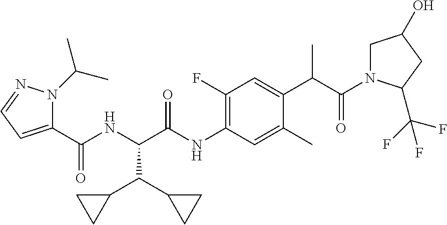 [SMILES: Cc1cc(NC(=O)[C@@H](NC(=O)c2ccnn2C(C)C)C(C2CC2)C2CC2)c(F)cc1C(C)C(=O)N1CC(O)CC1C(F)(F)F]

172 [M + H] = 622.2 m/z
Single diastereomer with underdetermined
absolute stereochemistry

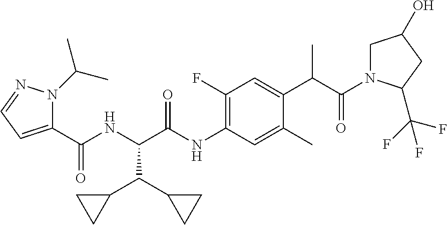 [SMILES: Cc1cc(NC(=O)[C@@H](NC(=O)c2ccnn2C(C)C)C(C2CC2)C2CC2)c(F)cc1C(C)C(=O)N1CC(O)CC1C(F)(F)F]

173 [M + H] = 622.2 m/z
Single diastereomer with underdetermined
absolute stereochemistry

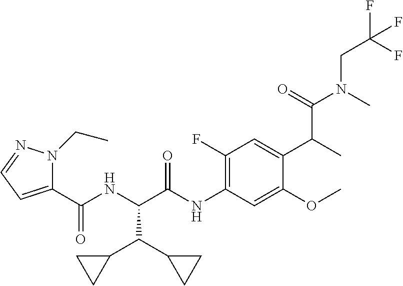 [SMILES: CCn1nccc1C(=O)N[C@H](C(=O)Nc1cc(OC)c(C(C)C(=O)N(C)CC(F)(F)F)cc1F)C(C1CC1)C1CC1]

174 [M + H] = 582.4 m/z

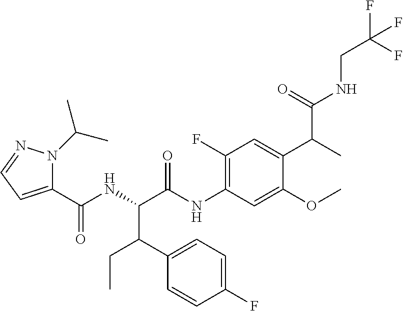 [SMILES: CCC(c1ccc(F)cc1)[C@H](NC(=O)c1ccnn1C(C)C)C(=O)Nc1cc(OC)c(C(C)C(=O)NCC(F)(F)F)cc1F]

175 [M + H] = 624.3 m/z

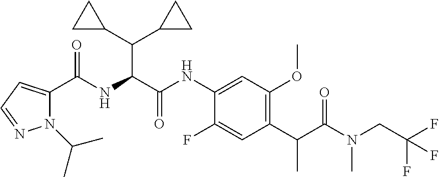 [SMILES: COc1cc(NC(=O)[C@@H](NC(=O)c2ccnn2C(C)C)C(C2CC2)C2CC2)c(F)cc1C(C)C(=O)N(C)CC(F)(F)F]

176 [M + H] = 596.4 m/z

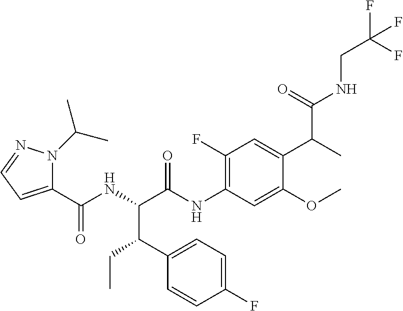 [SMILES: CC[C@@H](c1ccc(F)cc1)[C@H](NC(=O)c1ccnn1C(C)C)C(=O)Nc1cc(OC)c(C(C)C(=O)NCC(F)(F)F)cc1F]

177 [M + H] = 638.3 m/z

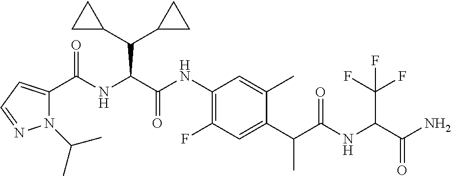 [SMILES: Cc1cc(NC(=O)[C@@H](NC(=O)c2ccnn2C(C)C)C(C2CC2)C2CC2)c(F)cc1C(C)C(=O)NC(C(N)=O)C(F)(F)F]

178 [M + H] = 609.4 m/z

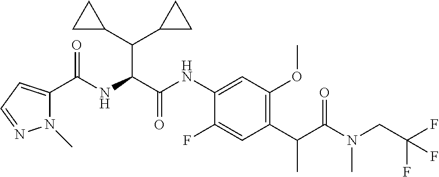 [SMILES: COc1cc(NC(=O)[C@@H](NC(=O)c2ccnn2C)C(C2CC2)C2CC2)c(F)cc1C(C)C(=O)N(C)CC(F)(F)F]

179 [M + H] = 568.3 m/z

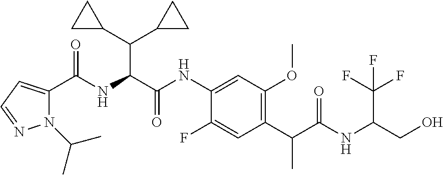 [SMILES: COc1cc(NC(=O)[C@@H](NC(=O)c2ccnn2C(C)C)C(C2CC2)C2CC2)c(F)cc1C(C)C(=O)NC(CO)C(F)(F)F]

180 [M + H] = 612.2 m/z
Single diastereomer with underdetermined
absolute stereochemistry

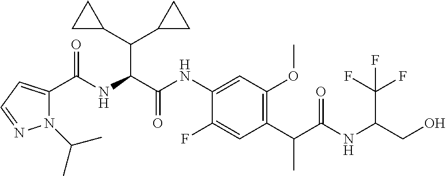 [SMILES: COc1cc(NC(=O)[C@@H](NC(=O)c2ccnn2C(C)C)C(C2CC2)C2CC2)c(F)cc1C(C)C(=O)NC(CO)C(F)(F)F]

181 [M + H] = 612.4 m/z
Single diastereomer with underdetermined
absolute stereochemistry

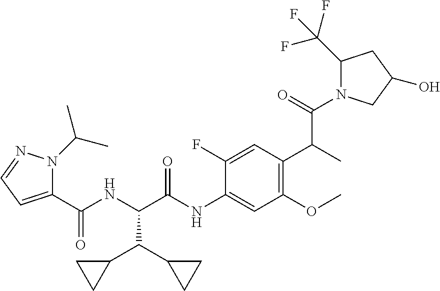 [SMILES: COc1cc(NC(=O)[C@@H](NC(=O)c2ccnn2C(C)C)C(C2CC2)C2CC2)c(F)cc1C(C)C(=O)N1CC(O)CC1C(F)(F)F]

182 [M + H] = 638.5 m/z
Single diastereomer with underdetermined
absolute stereochemistry

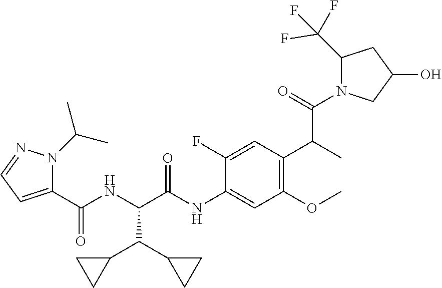 [SMILES: COc1cc(NC(=O)[C@@H](NC(=O)c2ccnn2C(C)C)C(C2CC2)C2CC2)c(F)cc1C(C)C(=O)N1CC(O)CC1C(F)(F)F]

183 [M + H] = 638.5 m/z
Single diastereomer with underdetermined
absolute stereochemistry

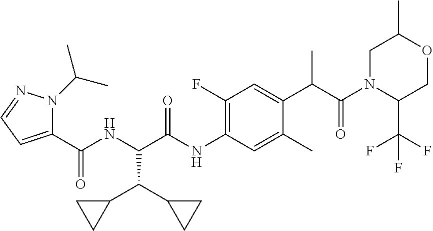

184 [M + H] = 636.5 m/z

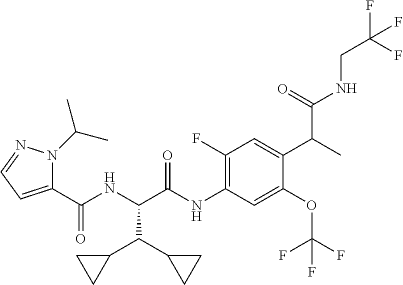 [SMILES: CC(C(=O)NCC(F)(F)F)c1cc(F)c(NC(=O)[C@@H](NC(=O)c2ccnn2C(C)C)C(C2CC2)C2CC2)cc1OC(F)(F)F]

185 [M + H] = 636.4 m/z
Single diastereomer with underdetermined
absolute stereochemistry

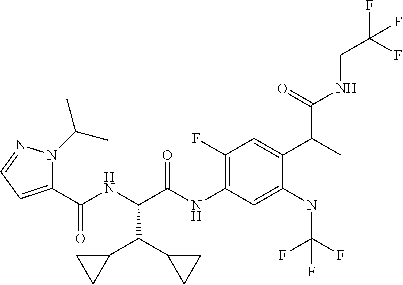 [SMILES: CC(C(=O)NCC(F)(F)F)c1cc(F)c(NC(=O)[C@@H](NC(=O)c2ccnn2C(C)C)C(C2CC2)C2CC2)cc1NC(F)(F)F]

186 [M + H] = 636.4 m/z
Single diastereomer with underdetermined
absolute stereochemistry

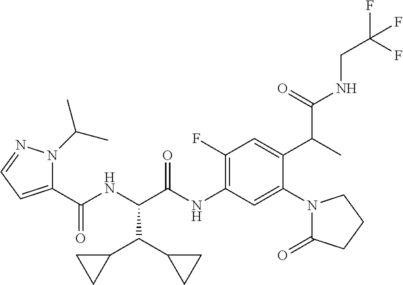 [SMILES: CC(C(=O)NCC(F)(F)F)c1cc(F)c(NC(=O)[C@@H](NC(=O)c2ccnn2C(C)C)C(C2CC2)C2CC2)cc1N1CCCC1=O]

187 [M + H] = 635.4 m/z

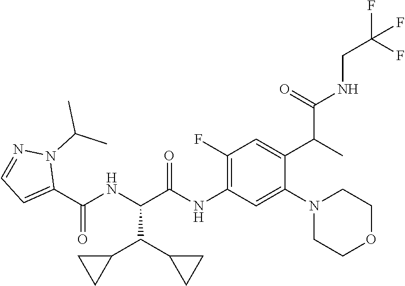 [SMILES: CC(C(=O)NCC(F)(F)F)c1cc(F)c(NC(=O)[C@@H](NC(=O)c2ccnn2C(C)C)C(C2CC2)C2CC2)cc1N1CCOCC1]

188 [M + H] = 637.4 m/z

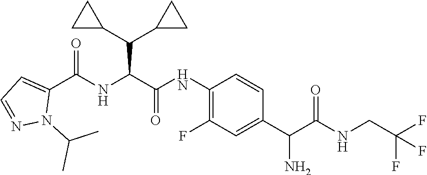 [SMILES: CC(C)n1nccc1C(=O)N[C@H](C(=O)Nc1ccc(C(N)C(=O)NCC(F)(F)F)cc1F)C(C1CC1)C1CC1]

189 [M + H] = 553.4 m/z
Single diastereomer with underdetermined
absolute stereochemistry

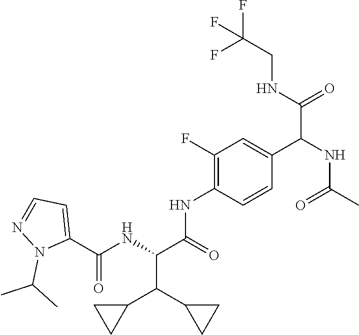 [SMILES: CC(=O)NC(C(=O)NCC(F)(F)F)c1ccc(NC(=O)[C@@H](NC(=O)c2ccnn2C(C)C)C(C2CC2)C2CC2)c(F)c1]

190 [M + H] = 595.4 m/z

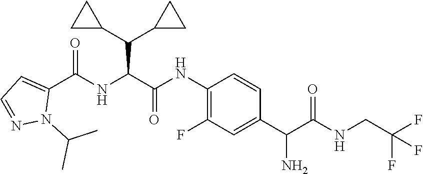 [SMILES: CC(C)n1nccc1C(=O)N[C@H](C(=O)Nc1ccc(C(N)C(=O)NCC(F)(F)F)cc1F)C(C1CC1)C1CC1]

191 [M + H] = 553.4 m/z
Single diastereomer with underdetermined
absolute stereochemistry

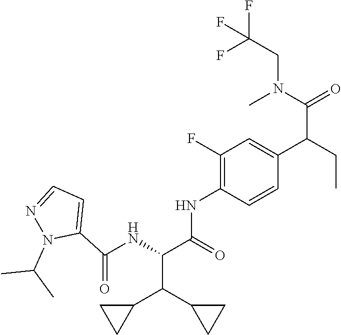 [SMILES: CCC(C(=O)N(C)CC(F)(F)F)c1ccc(NC(=O)[C@@H](NC(=O)c2ccnn2C(C)C)C(C2CC2)C2CC2)c(F)c1]

192 [M + H] = 580.3 m/z

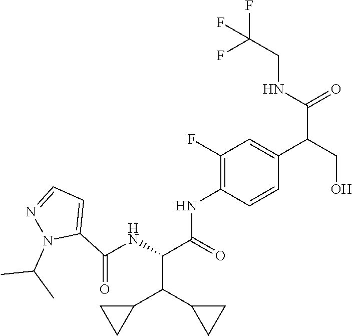 [SMILES: CC(C)n1nccc1C(=O)N[C@H](C(=O)Nc1ccc(C(CO)C(=O)NCC(F)(F)F)cc1F)C(C1CC1)C1CC1]

193 [M + H] = 568.3 m/z

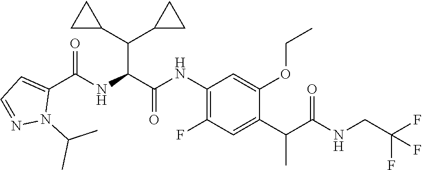 [SMILES: CCOc1cc(NC(=O)[C@@H](NC(=O)c2ccnn2C(C)C)C(C2CC2)C2CC2)c(F)cc1C(C)C(=O)NCC(F)(F)F]

194 [M + H] = 596.4 m/z

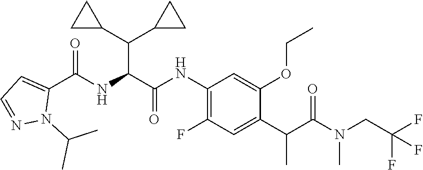 [SMILES: CCOc1cc(NC(=O)[C@@H](NC(=O)c2ccnn2C(C)C)C(C2CC2)C2CC2)c(F)cc1C(C)C(=O)N(C)CC(F)(F)F]

195 [M + H] = 610.4 m/z

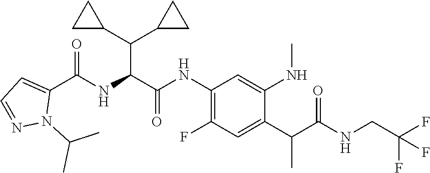 [SMILES: CNc1cc(NC(=O)[C@@H](NC(=O)c2ccnn2C(C)C)C(C2CC2)C2CC2)c(F)cc1C(C)C(=O)NCC(F)(F)F]

196 [M + H] = 581.5 m/z

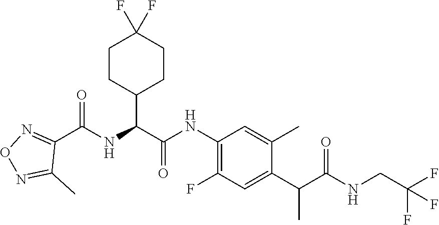 [SMILES: Cc1cc(NC(=O)[C@@H](NC(=O)c2nonc2C)C2CCC(F)(F)CC2)c(F)cc1C(C)C(=O)NCC(F)(F)F]

197 [M + H₃O] = 581.3 m/z

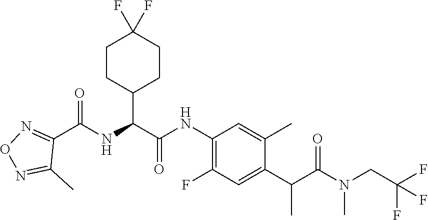 [SMILES: Cc1cc(NC(=O)[C@@H](NC(=O)c2nonc2C)C2CCC(F)(F)CC2)c(F)cc1C(C)C(=O)N(C)CC(F)(F)F]

198 [M + H] = 578.3 m/z

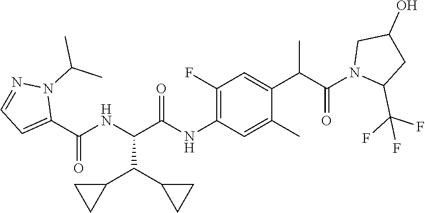 [SMILES: Cc1cc(NC(=O)[C@@H](NC(=O)c2ccnn2C(C)C)C(C2CC2)C2CC2)c(F)cc1C(C)C(=O)N1CC(O)CC1C(F)(F)F]

199 [M + H] = 622.2 m/z
Single diastereomer with underdetermined
absolute stereochemistry

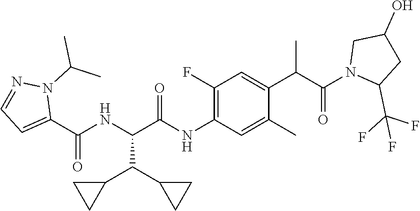 [SMILES: Cc1cc(NC(=O)[C@@H](NC(=O)c2ccnn2C(C)C)C(C2CC2)C2CC2)c(F)cc1C(C)C(=O)N1CC(O)CC1C(F)(F)F]

200 [M + H] = 622.2 m/z
Single diastereomer with underdetermined
absolute stereochemistry

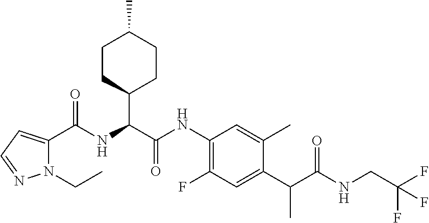 [SMILES: CCn1nccc1C(=O)N[C@H](C(=O)Nc1cc(C)c(C(C)C(=O)NCC(F)(F)F)cc1F)[C@H]1CC[C@H](C)CC1]

210 [M + H] = 554.2 m/z

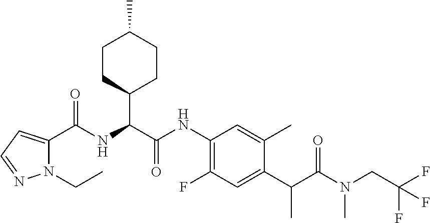 [SMILES: CCn1nccc1C(=O)N[C@H](C(=O)Nc1cc(C)c(C(C)C(=O)N(C)CC(F)(F)F)cc1F)[C@H]1CC[C@H](C)CC1]

202 [M + H] = 568.2 m/z

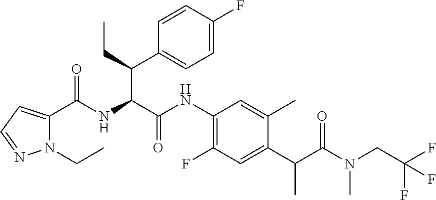 [SMILES: CC[C@@H](c1ccc(F)cc1)[C@H](NC(=O)c1ccnn1CC)C(=O)Nc1cc(C)c(C(C)C(=O)N(C)CC(F)(F)F)cc1F]

203 [M + H] = 608.2 m/z

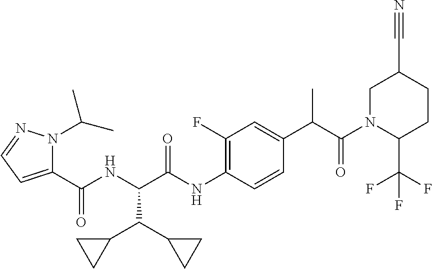 [SMILES: CC(C(=O)N1CC(C#N)CCC1C(F)(F)F)c1ccc(NC(=O)[C@@H](NC(=O)c2ccnn2C(C)C)C(C2CC2)C2CC2)c(F)c1]

204 [M + H] = 631.2 m/z

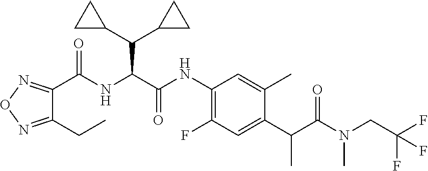 [SMILES: CCc1nonc1C(=O)N[C@H](C(=O)Nc1cc(C)c(C(C)C(=O)N(C)CC(F)(F)F)cc1F)C(C1CC1)C1CC1]

205 [M + H] = 568.2 m/z

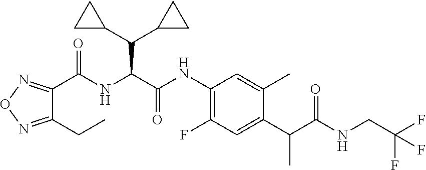 [SMILES: CCc1nonc1C(=O)N[C@H](C(=O)Nc1cc(C)c(C(C)C(=O)NCC(F)(F)F)cc1F)C(C1CC1)C1CC1]

206 [M + H] = 554.1 m/z

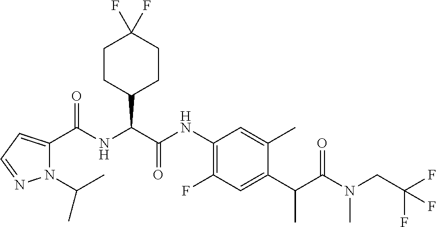 [SMILES: Cc1cc(NC(=O)[C@@H](NC(=O)c2ccnn2C(C)C)C2CCC(F)(F)CC2)c(F)cc1C(C)C(=O)N(C)CC(F)(F)F]

207 [M + H] = 604.2 m/z

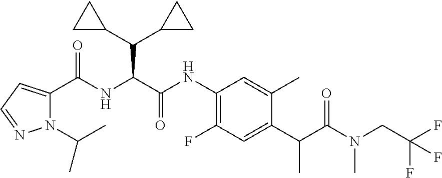 [SMILES: Cc1cc(NC(=O)[C@@H](NC(=O)c2ccnn2C(C)C)C(C2CC2)C2CC2)c(F)cc1C(C)C(=O)N(C)CC(F)(F)F]

208 [M + H] = 580.2 m/z

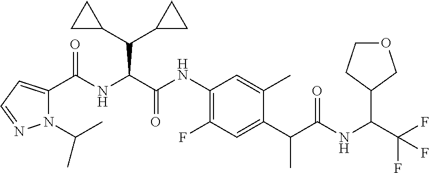 [SMILES: Cc1cc(NC(=O)[C@@H](NC(=O)c2ccnn2C(C)C)C(C2CC2)C2CC2)c(F)cc1C(C)C(=O)NC(C1CCOC1)C(F)(F)F]

209 [M + H] = 636.2 m/z

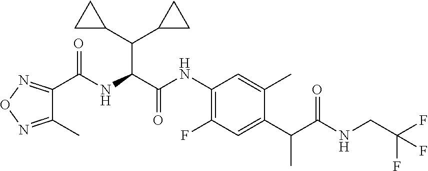 [SMILES: Cc1cc(NC(=O)[C@@H](NC(=O)c2nonc2C)C(C2CC2)C2CC2)c(F)cc1C(C)C(=O)NCC(F)(F)F]

210 [M + H] = 539.9 m/z

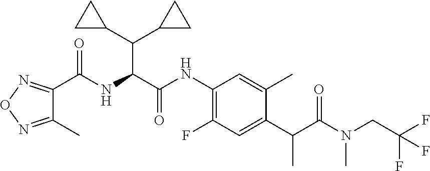 [SMILES: Cc1cc(NC(=O)[C@@H](NC(=O)c2nonc2C)C(C2CC2)C2CC2)c(F)cc1C(C)C(=O)N(C)CC(F)(F)F]

211 [M + H] = 553.9 m/z

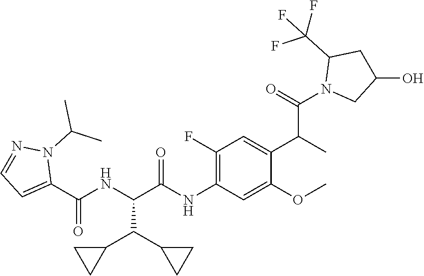 [SMILES: COc1cc(NC(=O)[C@@H](NC(=O)c2ccnn2C(C)C)C(C2CC2)C2CC2)c(F)cc1C(C)C(=O)N1CC(O)CC1C(F)(F)F]

212 [M + H] = 638.4 m/z
Single diastereomer with underdetermined
absolute stereochemistry

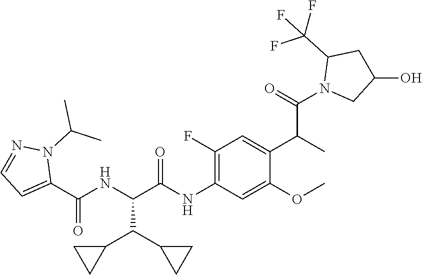 [SMILES: COc1cc(NC(=O)[C@@H](NC(=O)c2ccnn2C(C)C)C(C2CC2)C2CC2)c(F)cc1C(C)C(=O)N1CC(O)CC1C(F)(F)F]

213 [M + H] = 638.4 m/z
Single diastereomer with underdetermined
absolute stereochemistry

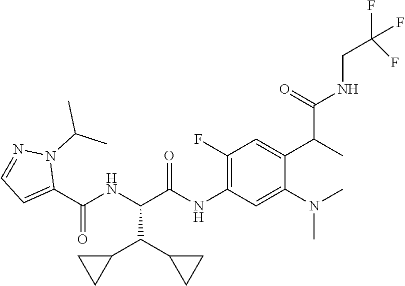 [SMILES: CC(C(=O)NCC(F)(F)F)c1cc(F)c(NC(=O)[C@@H](NC(=O)c2ccnn2C(C)C)C(C2CC2)C2CC2)cc1N(C)C]

214 [M + H] = 595.3 m/z

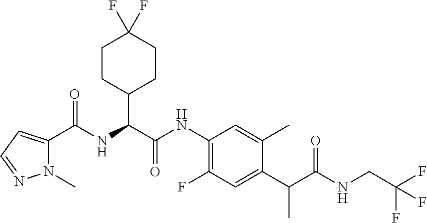 [SMILES: Cc1cc(NC(=O)[C@@H](NC(=O)c2ccnn2C)C2CCC(F)(F)CC2)c(F)cc1C(C)C(=O)NCC(F)(F)F]

215 [M + H] = 562.1 m/z

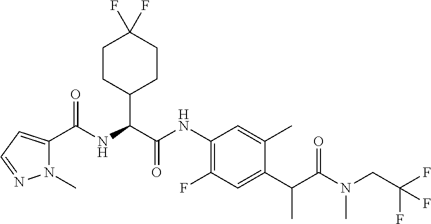 [SMILES: Cc1cc(NC(=O)[C@@H](NC(=O)c2ccnn2C)C2CCC(F)(F)CC2)c(F)cc1C(C)C(=O)N(C)CC(F)(F)F]

216 [M + H] = 576.2 m/z

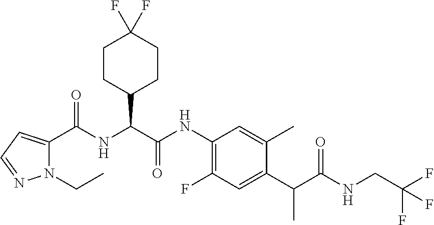 [SMILES: CCn1nccc1C(=O)N[C@H](C(=O)Nc1cc(C)c(C(C)C(=O)NCC(F)(F)F)cc1F)C1CCC(F)(F)CC1]

217 [M + H] = 576.1 m/z

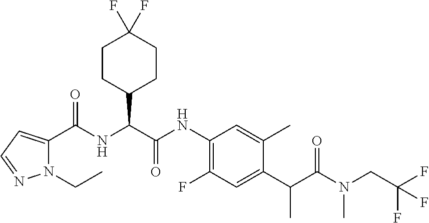 [SMILES: CCn1nccc1C(=O)N[C@H](C(=O)Nc1cc(C)c(C(C)C(=O)N(C)CC(F)(F)F)cc1F)C1CCC(F)(F)CC1]

218 [M + H] = 590.2 m/z

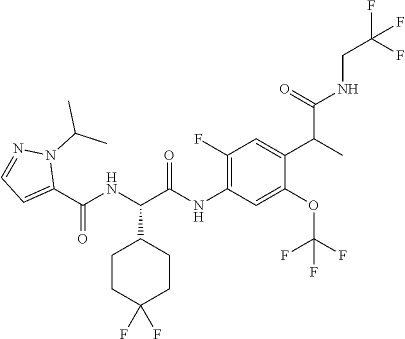 [SMILES: CC(C(=O)NCC(F)(F)F)c1cc(F)c(NC(=O)[C@@H](NC(=O)c2ccnn2C(C)C)C2CCC(F)(F)CC2)cc1OC(F)(F)F]

219 [M + H] = 660.2 m/z
Single diastereomer with underdetermined
absolute stereochemistry

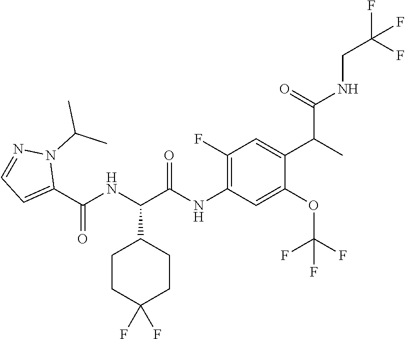 [SMILES: CC(C(=O)NCC(F)(F)F)c1cc(F)c(NC(=O)[C@@H](NC(=O)c2ccnn2C(C)C)C2CCC(F)(F)CC2)cc1OC(F)(F)F]

220 [M + H] = 660.3 m/z
Single diastereomer with underdetermined
absolute stereochemistry

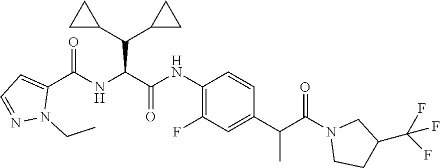 [SMILES: CCn1nccc1C(=O)N[C@H](C(=O)Nc1ccc(C(C)C(=O)N2CCC(C(F)(F)F)C2)cc1F)C(C1CC1)C1CC1]

221 [M + H] = 592.4 m/z

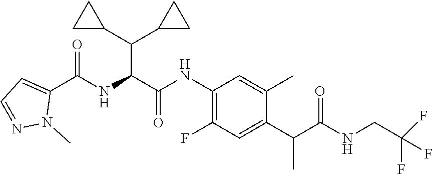 [SMILES: Cc1cc(NC(=O)[C@@H](NC(=O)c2ccnn2C)C(C2CC2)C2CC2)c(F)cc1C(C)C(=O)NCC(F)(F)F]

222 [M + H] = 538.1 m/z

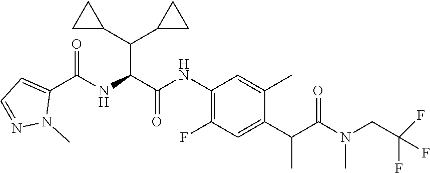 [SMILES: Cc1cc(NC(=O)[C@@H](NC(=O)c2ccnn2C)C(C2CC2)C2CC2)c(F)cc1C(C)C(=O)N(C)CC(F)(F)F]

223 [M + H] = 552.2 m/z

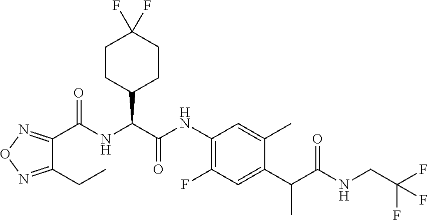 [SMILES: CCc1nonc1C(=O)N[C@H](C(=O)Nc1cc(C)c(C(C)C(=O)NCC(F)(F)F)cc1F)C1CCC(F)(F)CC1]

224 [M + H] = 578.2 m/z

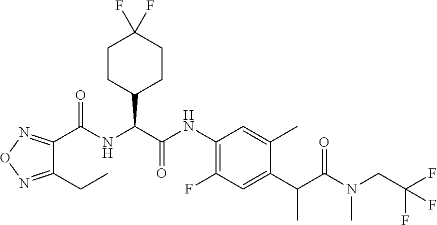 [SMILES: CCc1nonc1C(=O)N[C@H](C(=O)Nc1cc(C)c(C(C)C(=O)N(C)CC(F)(F)F)cc1F)C1CCC(F)(F)CC1]

225 [M + H] = 592.2 m/z

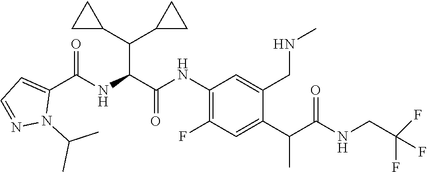 [SMILES: CNCc1cc(NC(=O)[C@@H](NC(=O)c2ccnn2C(C)C)C(C2CC2)C2CC2)c(F)cc1C(C)C(=O)NCC(F)(F)F]

226 [M + H] = 595.3 m/

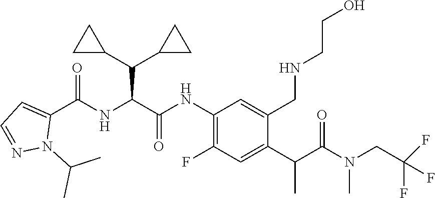 [SMILES: CC(C(=O)N(C)CC(F)(F)F)c1cc(F)c(NC(=O)[C@@H](NC(=O)c2ccnn2C(C)C)C(C2CC2)C2CC2)cc1CNCCO]

227 [M + H] = 625.4 m/z

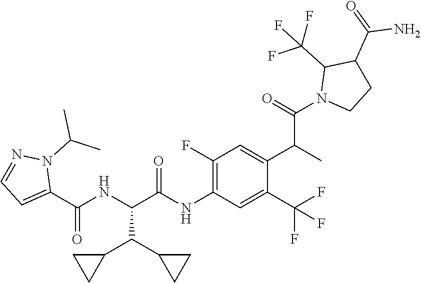 [SMILES: CC(C(=O)N1CCC(C(N)=O)C1C(F)(F)F)c1cc(F)c(NC(=O)[C@@H](NC(=O)c2ccnn2C(C)C)C(C2CC2)C2CC2)cc1C(F)(F)F]

228 [M + H] = 703.2 m/z

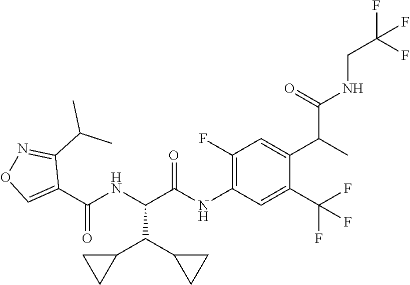 [SMILES: CC(C)c1nocc1C(=O)N[C@H](C(=O)Nc1cc(C(F)(F)F)c(C(C)C(=O)NCC(F)(F)F)cc1F)C(C1CC1)C1CC1]

229 [M + H] = 621.2 m/z

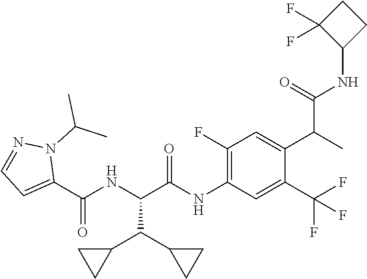 [SMILES: CC(C(=O)NC1CCC1(F)F)c1cc(F)c(NC(=O)[C@@H](NC(=O)c2ccnn2C(C)C)C(C2CC2)C2CC2)cc1C(F)(F)F]

230 [M + H] = 628.3 m/z
Single diastereomer with underdetermined
absolute stereochemistry

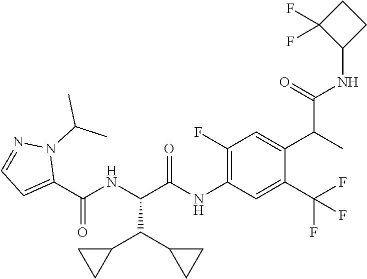 [SMILES: CC(C(=O)NC1CCC1(F)F)c1cc(F)c(NC(=O)[C@@H](NC(=O)c2ccnn2C(C)C)C(C2CC2)C2CC2)cc1C(F)(F)F]

231 [M + H] = 628.3 m/z
Single diastereomer with underdetermined
absolute stereochemistry

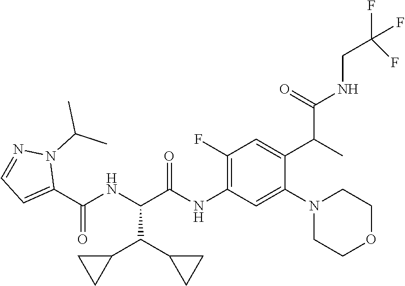 [SMILES: CC(C(=O)NCC(F)(F)F)c1cc(F)c(NC(=O)[C@@H](NC(=O)c2ccnn2C(C)C)C(C2CC2)C2CC2)cc1N1CCOCC1]

232 [M + H] = 637.4 m/z

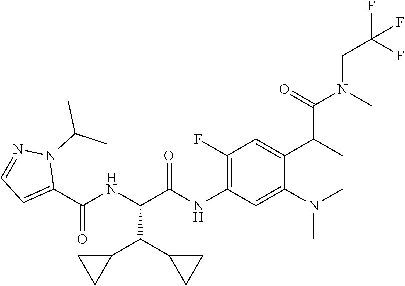 [SMILES: CC(C(=O)N(C)CC(F)(F)F)c1cc(F)c(NC(=O)[C@@H](NC(=O)c2ccnn2C(C)C)C(C2CC2)C2CC2)cc1N(C)C]

233 [M + H] = 609.5 m/z

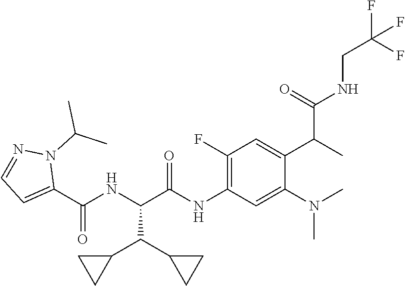 [SMILES: CC(C(=O)NCC(F)(F)F)c1cc(F)c(NC(=O)[C@@H](NC(=O)c2ccnn2C(C)C)C(C2CC2)C2CC2)cc1N(C)C]

234 [M + H] = 635.4 m/z

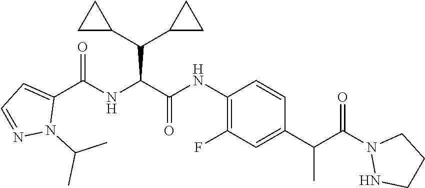 [SMILES: CC(C(=O)N1CCCN1)c1ccc(NC(=O)[C@@H](NC(=O)c2ccnn2C(C)C)C(C2CC2)C2CC2)c(F)c1]

235 [M + H] = 525 m/z

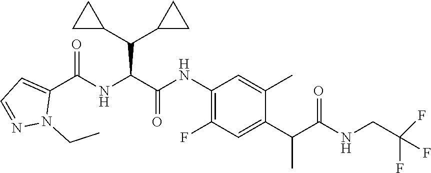 [SMILES: CCn1nccc1C(=O)N[C@H](C(=O)Nc1cc(C)c(C(C)C(=O)NCC(F)(F)F)cc1F)C(C1CC1)C1CC1]

236 [M + H] = 552.2 m/z

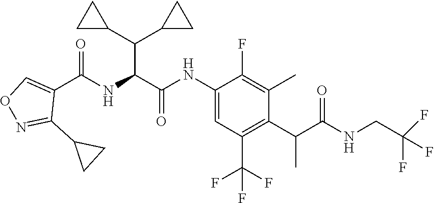 [SMILES: Cc1c(F)c(NC(=O)[C@@H](NC(=O)c2conc2C2CC2)C(C2CC2)C2CC2)cc(C(F)(F)F)c1C(C)C(=O)NCC(F)(F)F]

237 [M + H] = 619.3 m/z

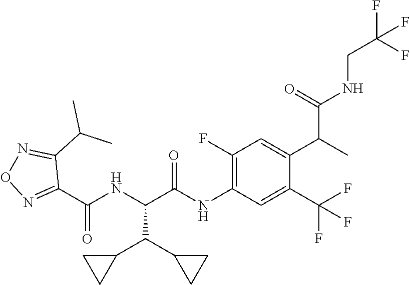 [SMILES: CC(C)c1nonc1C(=O)N[C@H](C(=O)Nc1cc(C(F)(F)F)c(C(C)C(=O)NCC(F)(F)F)cc1F)C(C1CC1)C1CC1]

238 [M + H] = 622.3 m/z

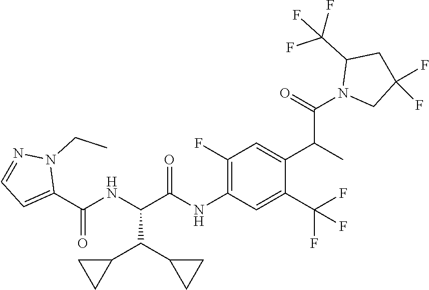 [SMILES: CCn1nccc1C(=O)N[C@H](C(=O)Nc1cc(C(F)(F)F)c(C(C)C(=O)N2CC(F)(F)CC2C(F)(F)F)cc1F)C(C1CC1)C1CC1]

239 [M + H] = 682.3 m/z

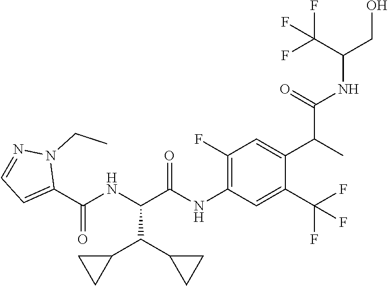 [SMILES: CCn1nccc1C(=O)N[C@H](C(=O)Nc1cc(C(F)(F)F)c(C(C)C(=O)NC(CO)C(F)(F)F)cc1F)C(C1CC1)C1CC1]

240 [M + H] = 636.3 m/z
Single diastereomer with underdetermined
absolute stereochemistry

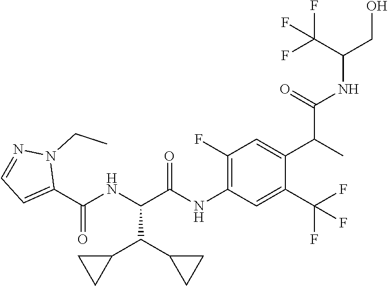 [SMILES: CCn1nccc1C(=O)N[C@H](C(=O)Nc1cc(C(F)(F)F)c(C(C)C(=O)NC(CO)C(F)(F)F)cc1F)C(C1CC1)C1CC1]

241 [M + H] = 636.3 m/z
Single diastereomer with underdetermined
absolute stereochemistry

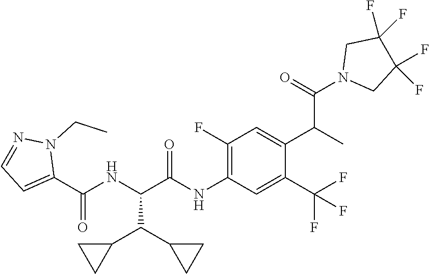 [SMILES: CCn1nccc1C(=O)N[C@H](C(=O)Nc1cc(C(F)(F)F)c(C(C)C(=O)N2CC(F)(F)C(F)(F)C2)cc1F)C(C1CC1)C1CC1]

242 [M + H] = 650.3 m/z

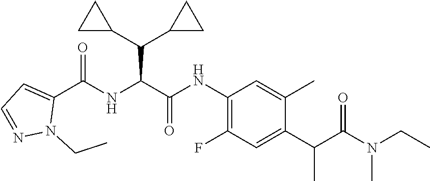 [SMILES: CCN(C)C(=O)C(C)c1cc(F)c(NC(=O)[C@@H](NC(=O)c2ccnn2CC)C(C2CC2)C2CC2)cc1C]

243 [M + H] = 512.4 m/z

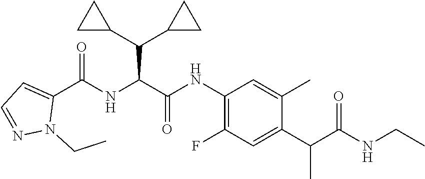 [SMILES: CCNC(=O)C(C)c1cc(F)c(NC(=O)[C@@H](NC(=O)c2ccnn2CC)C(C2CC2)C2CC2)cc1C]

244 [M + H] = 498.4 m/z

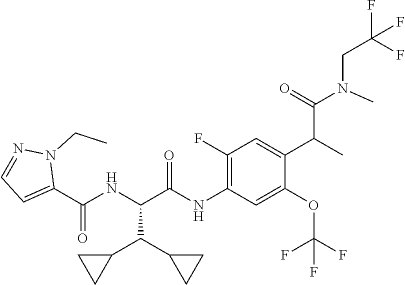 [SMILES: CCn1nccc1C(=O)N[C@H](C(=O)Nc1cc(OC(F)(F)F)c(C(C)C(=O)N(C)CC(F)(F)F)cc1F)C(C1CC1)C1CC1]

245 [M + H] = 636.3 m/z

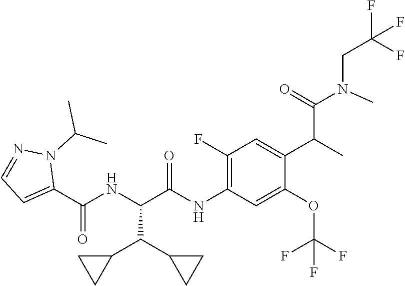 [SMILES: CC(C(=O)N(C)CC(F)(F)F)c1cc(F)c(NC(=O)[C@@H](NC(=O)c2ccnn2C(C)C)C(C2CC2)C2CC2)cc1OC(F)(F)F]

246 [M + H] = 650.4 m/z

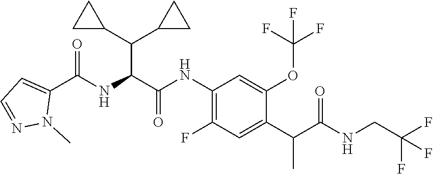 [SMILES: CC(C(=O)NCC(F)(F)F)c1cc(F)c(NC(=O)[C@@H](NC(=O)c2ccnn2C)C(C2CC2)C2CC2)cc1OC(F)(F)F]

247 [M + H] = 608.3 m/z

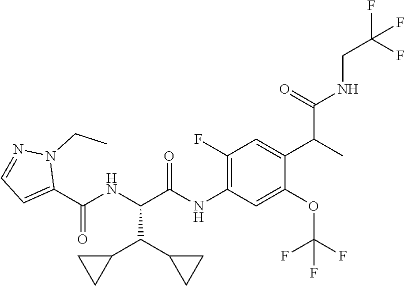 [SMILES: CCn1nccc1C(=O)N[C@H](C(=O)Nc1cc(OC(F)(F)F)c(C(C)C(=O)NCC(F)(F)F)cc1F)C(C1CC1)C1CC1]

248 [M + H] = 622.3 m/z

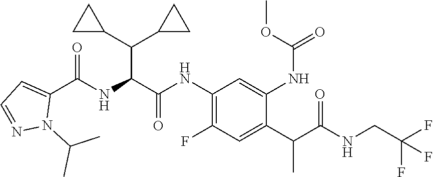 [SMILES: COC(=O)Nc1cc(NC(=O)[C@@H](NC(=O)c2ccnn2C(C)C)C(C2CC2)C2CC2)c(F)cc1C(C)C(=O)NCC(F)(F)F]

249 [M + H] = 625.4 m/z

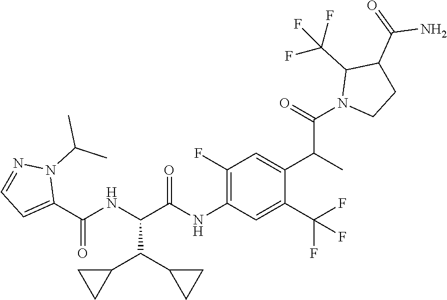 [SMILES: CC(C(=O)N1CCC(C(N)=O)C1C(F)(F)F)c1cc(F)c(NC(=O)[C@@H](NC(=O)c2ccnn2C(C)C)C(C2CC2)C2CC2)cc1C(F)(F)F]

250 [M + H] = 703.2 m/z

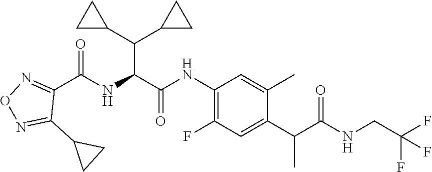 [SMILES: Cc1cc(NC(=O)[C@@H](NC(=O)c2nonc2C2CC2)C(C2CC2)C2CC2)c(F)cc1C(C)C(=O)NCC(F)(F)F]

251 [M + H] = 566.1 m/z

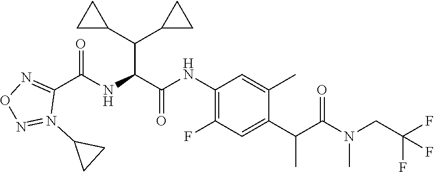

252 [M + H] = 580.2 m/z

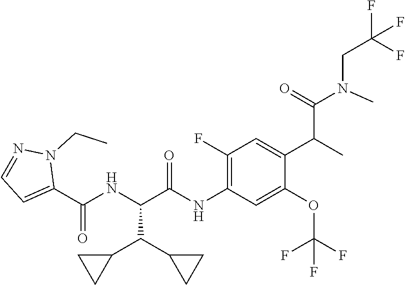 [SMILES: CCn1nccc1C(=O)N[C@H](C(=O)Nc1cc(OC(F)(F)F)c(C(C)C(=O)N(C)CC(F)(F)F)cc1F)C(C1CC1)C1CC1]

253 [M + H] = 636.3 m/z

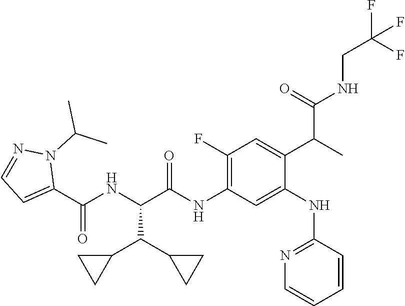 [SMILES: CC(C(=O)NCC(F)(F)F)c1cc(F)c(NC(=O)[C@@H](NC(=O)c2ccnn2C(C)C)C(C2CC2)C2CC2)cc1Nc1ccccn1]

254 [M + H] = 644.5 m/z

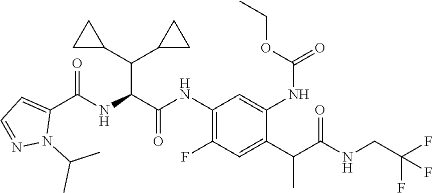 [SMILES: CCOC(=O)Nc1cc(NC(=O)[C@@H](NC(=O)c2ccnn2C(C)C)C(C2CC2)C2CC2)c(F)cc1C(C)C(=O)NCC(F)(F)F]

255 [M + H] = 639.4 m/z

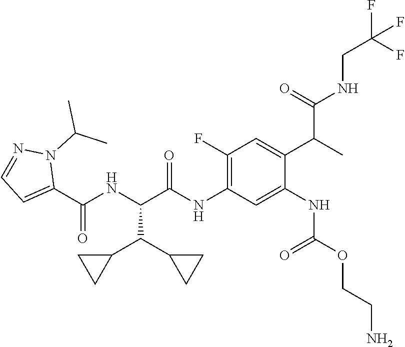 [SMILES: CC(C(=O)NCC(F)(F)F)c1cc(F)c(NC(=O)[C@@H](NC(=O)c2ccnn2C(C)C)C(C2CC2)C2CC2)cc1NC(=O)OCCN]

256 [M + H] = 654.6 m/z

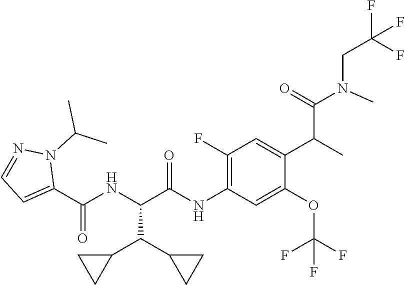 [SMILES: CC(C(=O)N(C)CC(F)(F)F)c1cc(F)c(NC(=O)[C@@H](NC(=O)c2ccnn2C(C)C)C(C2CC2)C2CC2)cc1OC(F)(F)F]

257 [M + H] = 650.4 m/z

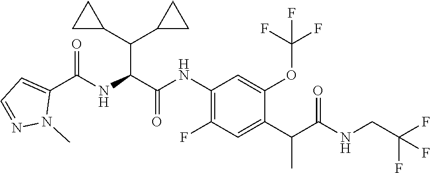 [SMILES: CC(C(=O)NCC(F)(F)F)c1cc(F)c(NC(=O)[C@@H](NC(=O)c2ccnn2C)C(C2CC2)C2CC2)cc1OC(F)(F)F]

258 [M + H] = 608.3 m/z

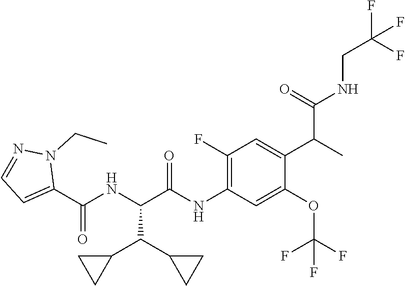 [SMILES: CCn1nccc1C(=O)N[C@H](C(=O)Nc1cc(OC(F)(F)F)c(C(C)C(=O)NCC(F)(F)F)cc1F)C(C1CC1)C1CC1]

259 [M + H] = 622.3 m/z

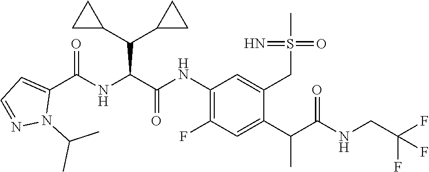 [SMILES: CC(C(=O)NCC(F)(F)F)c1cc(F)c(NC(=O)[C@@H](NC(=O)c2ccnn2C(C)C)C(C2CC2)C2CC2)cc1CS(C)(=N)=O]

260 [M + H] = 643.3 m/z

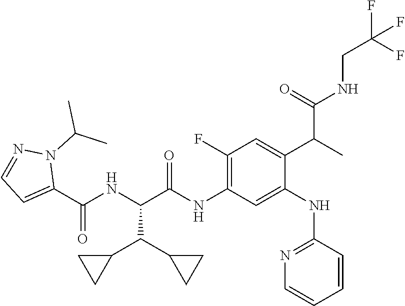 [SMILES: CC(C(=O)NCC(F)(F)F)c1cc(F)c(NC(=O)[C@@H](NC(=O)c2ccnn2C(C)C)C(C2CC2)C2CC2)cc1Nc1ccccn1]

261 [M + H] = 644.4 m/z
Single diastereomer with underdetermined
absolute stereochemistry

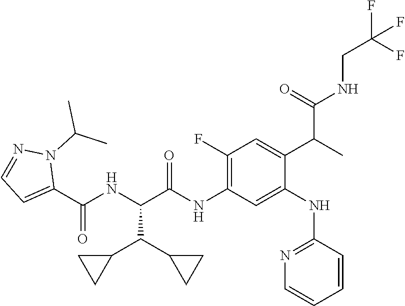 [SMILES: CC(C(=O)NCC(F)(F)F)c1cc(F)c(NC(=O)[C@@H](NC(=O)c2ccnn2C(C)C)C(C2CC2)C2CC2)cc1Nc1ccccn1]

262 [M + H] = 644.4 m/z
Single diastereomer with underdetermined
absolute stereochemistry

### Example 177

The HEK-Blue #NC108637 wash used 17A/A inhibition assay's. Cells were grown and prepared for assays according to the manufacturer's instructions. This cell line consists of HEK 293 cells that were designed to expressed IL-17R^{A}, IL-17RC, and the ActI adapter molecule, the combination of which. when stimulated by IL-17A/A activates a NFtcB promoter and drives expression of a recombinant Secreted Alkaline Phosphatase (SEAP) geneprotein. Media from the cells is then added to a development reagent (Quanti-Blue Substrate, Fisher #NC971 1613), and read at A₆₃₀.

Compounds were titrated in DMSO. with a top final compound concentration of 10 uM, 1 uM, or 0.3 uM, and added to the cells immediately before adding IL-17A/A (Genscript #Z03228). The cells, compound, and IL-17A/A were then incubated for 20 hours before media was removed for SEAP analysis. The resulting inhibition curve was then analyzed using Graphpad Prism 7.0, and IC₅₀ values were determined using a 4-parameter nonlinear fit. DMSO was added to a universal final concentration of 0.1% to optimze background.

Table 2 includes IC₅₀ values for IL-17A/A inhibition of selected compounds-, with compounds having a IC₅₀ of A<100 nW. B 100-1000 nM; and C—>1000 WM

TABLE 2
IL-17 A/A Inhibition Data for selected compounds

| Compound | IL-17 A/A |
|---|---|
| number | Inhibition |
| 1 | B |
| 2 | B |
| 3 | B |
| 4 | A |
| 5 | C |
| 6 | B |
| 7 | B |
| 8 | A |
| 9 | C |
| 10 | B |
| 11 | A |
| 12 | B |
| 13 | B |
| 14 | B |
| 15 | B |
| 16 | C |
| 17 | B |
| 18 | A |
| 19 | B |
| 20 | B |
| 21 | B |
| 22 | B |
| 23 | B |
| 24 | B |
| 25 | B |
| 26 | B |
| 27 | C |
| 28 | B |
| 29 | A |
| 30 | B |
| 31 | C |
| 32 | C |
| 33 | C |
| 34 | B |
| 35 | C |
| 36 | C |
| 37 | C |
| 38 | B |
| 39 | C |
| 40 | B |
| 41 | B |
| 42 | B |
| 43 | C |
| 44 | B |
| 45 | B |
| 46 | C |
| 47 | A |
| 48 | B |
| 49 | B |
| 50 | B |
| 51 | B |
| 52 | C |
| 53 | B |
| 54 | C |
| 55 | B |
| 56 | B |
| 57 | B |
| 58 | B |
| 59 | C |
| 60 | A |
| 61 | A |
| 62 | B |
| 63 | A |
| 64 | B |
| 65 | C |
| 66 | B |
| 67 | A |
| 68 | A |
| 69 | C |
| 70 | B |
| 71 | A |
| 72 | B |
| 73 | C |
| 74 | B |
| 75 | B |
| 76 | A |
| 77 | B |
| 78 | A |
| 79 | B |
| 80 | A |
| 81 | 0 |
| 82 | A |
| 83 | C |
| 84 | A |
| 85 | B |
| 86 | B |
| 87 | B |
| 88 | B |
| 89 | B |
| 90 | B |
| 91 | C |
| 92 | B |
| 93 | B |
| 94 | B |
| 95 | B |
| 96 | C |
| 97 | C |
| 98 | A |
| 99 | C |
| 100 | C |
| 101 | C |
| 102 | C |
| 103 | A |
| 104 | C |
| 105 | A |
| 106 | C |
| 107 | B |
| 108 | B |
| 109 | B |
| 110 | A |
| 111 | A |
| 112 | B |
| 113 | B |
| 114 | C |
| 115 | B |
| 116 | C |
| 117 | B |
| 118 | C |
| 119 | B |
| 120 | A |
| 121 | C |
| 122 | C |
| 123 | A |
| 124 | A |
| 125 | C |
| 126 | B |
| 127 | A |
| 128 | B |
| 129 | C |
| 130 | C |
| 131 | B |
| 132 | C |
| 133 | C |
| 134 | B |
| 135 | B |
| 136 | C |
| 137 | A |
| 138 | C |
| 139 | B |
| 140 | C |
| 141 | C |
| 142 | A |
| 143 | B |
| 144 | C |
| 145 | C |
| 146 | C |
| 147 | C |
| 148 | C |
| 149 | B |
| 150 | C |
| 151 | B |
| 152 | B |
| 153 | B |
| 154 | B |
| 155 | C |
| 156 | B |
| 157 | B |
| 158 | A |
| 159 | A |
| 160 | A |
| 161 | A |
| 162 | A |
| 163 | A |
| 164 | A |
| 165 | B |
| 166 | B |
| 167 | B |
| 168 | B |
| 169 | A |
| 170 | C |
| 171 | B |
| 172 | B |
| 173 | A |
| 174 | A |
| 175 | B |
| 176 | A |
| 177 | B |
| 178 | B |
| 179 | A |
| 180 | A |
| 181 | B |
| 182 | C |
| 183 | C |
| 184 | A |
| 185 | A |
| 186 | B |
| 187 | B |
| 188 | A |
| 189 | C |
| 190 | C |
| 191 | B |
| 192 | A |
| 193 | C |
| 194 | B |
| 195 | A |
| 196 | C |
| 197 | C |
| 198 | B |
| 199 | A |
| 200 | B |
| 201 | C |
| 202 | A |
| 203 | A |
| 204 | B |
| 205 | A |
| 206 | B |
| 207 | A |
| 208 | A |
| 209 | A |
| 210 | B |
| 211 | A |
| 212 | A |
| 213 | B |
| 214 | C |
| 215 | C |
| 216 | B |
| 217 | C |
| 218 | A |
| 219 | B |
| 220 | C |
| 221 | B |
| 222 | C |
| 223 | A |
| 224 | B |
| 225 | B |
| 226 | B |
| 227 | B |
| 228 | B |
| 229 | C |
| 230 | B |
| 231 | B |
| 232 | C |
| 233 | C |
| 234 | C |
| 235 | C |
| 236 | A |
| 237 | C |
| 238 | B |
| 239 | B |
| 240 | A |
| 241 | C |
| 242 | B |
| 243 | B |
| 244 | C |
| 245 | B |
| 246 | C |
| 247 | C |
| 248 | C |
| 249 | B |
| 250 | A |
| 251 | A |
| 252 | A |
| 253 | A |
| 254 | C |
| 255 | C |
| 256 | B |
| 257 | A |
| 258 | A |
| 259 | A |
| 260 | B |
| 261 | C |
| 262 | C |

What is claimed is:

1. A compound represented by the structure of Formula I: 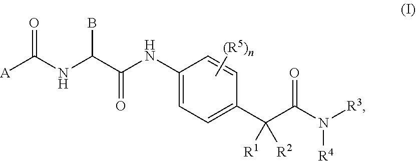
or a pharmaceutically acceptable salt thereof, wherein:
A is selected from a 5- to 6-membered heterocycle optionally substituted with one more substituents independently selected from (i), (ii), and (iii):
(i) halogen, —OR¹¹, —SR¹¹, —N(R¹¹)₂, —C(O)R¹¹, —C(O)OR¹¹, —OC(O)R¹¹, —OC(O)N(R¹¹)₂, —C(O)N(R¹¹), —N(R¹¹)C(O)R¹¹, —N(R¹¹)C(O)OR¹¹, —N(R¹¹)C(O)N(R¹¹)₂, —N(R¹¹)S(O)₂(R¹¹), —S(O)R¹¹, —S(O)₂R¹¹, —S(O)₂N(R¹¹)₂, —NO₂, and —CN;
(ii)C-to alkyl optionally substituted with one or more substituents independently selected from:
halogen, —OR¹¹, —SR¹¹, —N(R¹¹)₂, —C(O)R¹¹, —C(O)OR¹¹, —OC(O)R¹¹, —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹¹)₂, —N(R¹¹)S(O)₂(R¹¹), —S(O)R¹¹, —S(O)₂R¹¹, —S(O)₂N(R¹¹)₂, —NO₂, ═O, —CN; and
C₃₋₁₀ carbocycle and 3- to 10-membered heterocycle each of which is optionally substituted with one or more substituents selected from: halogen, —OR¹¹, —SR¹¹, —N(R¹¹)₂, —C(O)R¹¹, —C(O)OR¹¹, —OC(O)R¹¹, —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹¹)₂, —N(R¹¹)S(O)₂(R¹¹), —S(O)R¹¹, —S(O)₂R¹¹, —S(O)₂N(R¹¹)₂, —NO₂, =O, and —CN; and
(iii) 3- to 10-membered heterocycle optionally substituted with one or more substituents independently selected from:
halogen, —OR¹¹, —SR¹¹, —N(R¹¹)₂, —C(O)R¹¹, —C(O)OR¹¹, —OC(O)R¹¹, —OC(O)N(R¹¹)₂, —C(O)N(R¹¹)₂, —N(R^{11B})C(O)R¹¹, —N(R¹¹)C(O)OR¹², —N(R¹¹)C(O)N(R¹¹)₂, —N(R¹¹)S(O)₂(R¹¹), —S(O)R¹¹, —S(O)₂R¹¹, —S(O)₂N(R¹¹)₂, —NO₂, ═O, —CN; and
C₁₋₆ alkyl optionally substituted with one or more substituents independently selected from halogen, —OR¹¹, —SR¹¹, —N(R¹¹)₂, —C(O)R¹¹, —C(O)OR¹¹, —OC(O)R¹¹, —OC(O)N(Rh)z, —C(O)N(R¹¹)₂, —N(R¹¹)C(O)R¹¹, —N(R¹¹)C(O)OR¹¹, —N(R¹¹)C(O)N(R¹¹)₂, —N(R¹¹)S(O)₂(R¹¹), —S(O)R^{11B}, —S(O)₂R¹¹, —S(O)₂N(R¹¹)₂, —NO₂, ═O, and —CN;
B is selected from —CH(R^{A})(R^{B}) and C₃₋₁₀ carbocycle, wherein the C₃₋₁₀ carbocycle is optionally substituted with one or more substituents independently selected from:
halogen, —OR¹², —SR¹², —N(R¹²)₂, —C(O)R², —C(O)OR², —OC(O)R¹², —OC(O)N(R¹¹)₂, —C(O)N(R¹²)₂, —N(R¹²)C(O)R¹², —N(R¹²)C(O)OR¹², —N(R¹)C(O)N(R¹)₂, —N(R¹z)S(O)₂(R¹¹), —S(O)R¹², —S(O)₂R¹², —S(O)₂N(R¹¹)₂, —NO₂, ═O, and —CN; and
C₁₋₆ alkyl optionally substituted with one or more substituents independently selected from halogen, —OR¹², —SR², —N(R¹²)₂, —C(O)R², —C(O)OR¹², —OC(O)R¹², —OC(O)N(R¹)₂, —C(O)N(R¹²)₂, —N(R¹²)C(O)R¹², —N(R¹²)C(O)OR², —N(R¹¹)C(O)N(R¹¹)₂, —N(R^{1)S(O)}₂(R¹²), —S(O)R¹², —S(O)₂R¹², —S(O)₂N(R¹²)₂, —NO₂, ═O, and —CN;
R^{A} and R^{B} are each independently selected from:
halogen, —OR¹², —SR¹, —N(R¹¹)₂, —C(O)R¹³, —C(O)OR³, —OC(O)R¹³, —OC(O)N(R¹¹)₂, —C(O)N(R¹¹)₂, —N(R¹¹)C(O)R¹¹, —N(R¹¹)C(O)OR¹², —N(R¹¹)C(O)N(R¹³)₂, —N(R¹³)S(O)₂(R¹³), —S(O)R¹¹, —S(O)₂R¹³, —S(O)₂N(R¹¹)₂, —NO₂, and —CN;
C₁₋₆ alkyl optionally substituted with one or more substituents independently selected from halogen, —OR¹², —SR¹¹, —N(R¹¹)₂, —C(O)R¹¹, —C(O)OR¹¹, —OC(O)R¹³, —OC(O)N(R¹¹)₂, —C(O)N(R¹³)₂, —N(R¹¹)C(O)R¹³, —N(R¹³)C(O)OR¹³, —N(R^{1′})C(O)N(R^{1′})₂, —N(R¹³)₂S(O)₂(R¹¹), —S(O)R¹¹, —S(O)₂R^{11′}, —S(O)₂N(R¹¹)₂, —NO₂, and —CN; and
C₃₋₁₀ carbocycle optionally substituted with one or more substituents independently selected from halogen, —OR¹³, —SR¹¹, —N(R¹¹)₂, —C(O)R¹¹, —C(O)OR¹¹, —OC(O)R^{D}, —OC(O)N(R)z2, —C(O)N(R¹¹)₂, —N(R¹¹)C(O)R¹², —N(R¹³)C(O)OR¹¹, —N(R¹³)C(O)N(R¹³)₂, —N(R¹³)S(O)₂(R¹³), —S(O)R¹³, —S(O)₂R¹³, —S(O)₂N(R¹³)₂, —NO₂, and —CN;
R¹ and R² are each independently selected from (iv), (v), (vi) and (vii):
(iv) hydrogen;
(v) halogen, —OR¹⁴, —SR¹⁴, —N(R¹⁴)₂, —C(O)R¹⁴, —C(O)OR¹⁴, —OC(O)R¹¹, —OC(O)N(R¹⁴)₂, —C(O)N(R¹⁴)₂, —N(R¹⁴)C(O)R¹⁴, —N(R¹⁴)C(O)OR^{11B}, —N(R¹⁴)C(O)N(R¹⁴)₂, —N(R¹⁴)S(O)₂(R¹⁴), —S(O)R¹⁴, —S(O)₂R¹⁴, —S(O)₂N(R¹⁴)₂, —NO₂, and —CN;
(vi) C₁₋₆ alkyl optionally substituted with one or more substituents independently selected from:
halogen, —OR¹⁴, —SR¹⁴, —N(R¹⁴)₂, —C(O)R¹⁴, —C(O)OR¹⁴, —OC(O)R^{M}a, —OC(O)N(R¹⁴)₂, —C(O)N(R¹⁴)₂, —N(R¹⁴)C(O)R¹⁴, —N(R^{1′})C(O)OR¹⁴, —N(R¹⁴)C(O)N(R¹⁴)₂, —N(R¹⁴)S(O)₂(R¹⁴), —S(O)R¹⁴, —S(O)₂R¹⁴, —S(O)₂N(R¹⁴)₂, —NO₂, and —CN; and
3- to 10-membered heterocycle optionally substituted with one or more substituents independently selected from halogen, —OR^{14B}, —SR¹⁴, —N(R^{14B}), —C(O)R^{14B}, C(O)OR^{14B}, —OC(O)R^{14B}, —OC(O)N(R^{14B})₂—C(O)N(R^{14B})₂, —N(R^{14B})C(O)R^{14B}, —N(R¹⁴D)C(O)OR^{14B}, —N(R^{14B})C(O)N(R^{14B})₂, —N(R^{14B})S(O)₂(R^{14B}) —S(O)R^{14B}, —S(O)₂R^{14B}, —S(O)₂N(R^{14B})₂, —NO₂, and —CN; and
(vii) 3- to 10-membered heterocycle optionally substituted with one or more substituents independently selected from:
halogen, —OR^{14B}—SR^{14B}, —N(R^{14B})₂, —C(O)R^{14B}, —C(O)OR^{14B} —OC(O)R^{14B}, —OC(O)N(R^{14B}), —C(O)N(R^{14B})₂, —N(R^{14B})C(O)R^{14B}, —N(R^{14B})C(O)OR^{14B}, —N(R^{14B})C(O)N(R¹⁴n)₂, —N(R¹⁴)S(O)₂(R^{14B}), —S(O)R¹⁴n, —S(O2R^{14B}, —S(O)₂N(R^{4B})₂, —NO₂, and —CN;
R³ and R⁴ are each independently selected from (a), (b), (c), and (d):
(a) hydrogen;
(b) C₁ alkyl optionally substituted with one or more substituents independently selected from:
halogen, —OR¹¹, —SR¹¹, —N(R¹¹)₂, —C(O)R¹¹, —C(O)NH₂, —C(O)—O—C₁₋₆-alkyl, —OC(O)R¹¹, —OC(O)N(R¹¹)z, —N(R¹¹)C(O)R¹¹, —N(R¹¹)C(O)OR¹⁴, —N(R¹¹)C(O)N(R¹¹)₂, —N(R¹¹)S(O)₂(R¹¹), —S(O)R¹¹)₅, —S(O)₂R¹¹, —S(O)₂N(R¹¹)₅)₂, —NO₂, —CN;
C₃₋₁₀ carbocycle and 3- to 10-membered heterocycle any of which is optionally substituted with one or more substituents independently selected from halogen, C₁₋₄,haloalkyl, C₁₋₆ alkoxy, —OR¹¹, —SR¹¹, —N(R¹¹)₂, —C(O)R¹¹)₅, —C(O)OR¹¹, —OC(O)R¹⁴, —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹¹)₅)₂, —N(R¹¹)₅)S(O)₂(R¹¹), —S(O)R¹¹, —S(O)₂R^{1S}, —S(O)₂N(R¹¹)₂, —NO₂, and —CN;
(c) C₂i alkyl optionally substituted with one or more substituents independently selected from:
halogen, —OR¹¹, —SR¹¹, —N(R¹¹)₂, —C(O)R¹¹, —C(O)—O-Ct-₆-alkyl, —OC(O)R¹¹, —OC(O)N(R¹¹)₂—C(O)N(R¹¹)₂, —N(R¹¹)C(O)R¹¹, —N(R¹¹)C(O)OR¹¹, —N(R¹⁵)C(O)N(R¹⁵)₂, —N(R¹⁵)S(O)₂(R¹⁵), —S(O)R¹⁵, —S(O)₂R¹¹, —S(O)₂N(R¹¹)₅)₂, —NO₂, and —CN;
C₃₋₁₀ carbocycle and 3- to 10-membered heterocycle any of which is optionally substituted with one or more substituents independently selected from halogen, C₁₋₄haloalkyl, C₁₋₄ alkoxy, —OR¹¹, —SR¹¹, —N(R¹¹)₂, —C(O)R¹¹)_{S}, —C(O)OR¹¹, —OC(O)R¹”, —OC(O)N(R¹¹)₂, —C(O)N(R )₂, —N(R¹¹)C(O)R¹¹, —N(R¹⁵)C(O)OR¹⁵, —N(R¹⁵)C(O)N(R¹⁵)₅)₂, —N(R¹¹)₃)S(O)₂(R¹³), —S(O)R¹, —S(O)₂R¹⁴, —S(O)₂N(R¹¹)₂, —NO₂, and —CN; and
(d) C₃₋₁₀ carbocycle and 3- to 10-membered heterocycle any of which is optionally substituted with one or more substituents independently selected from:
halogen, —OR¹⁵, —SR¹¹, —N(R⁵)₂, —C(O)R¹S, —C(O)OR⁵, —OC(O)R⁵, —OC(O)N(R¹¹)₂, —C(O)N(R¹¹)₂, —N(R¹⁶)C(O)R¹⁵, —N(R¹⁵)C(O)OR⁵, —N(R¹⁴)C(O)N(R¹¹)₅)₂, —N(R¹¹)_{S})S(O)₂(R⁵), —S(O)R¹⁵, —S(O)₂R¹⁵, —S(O)₂N(R¹⁵), —NO₂, and —CN; and
C₁₋₆ alkyl optionally substituted with one or more substituents independently selected from halogen, —OR¹⁵, —SR¹⁵, —N(R¹¹)₂, —C(O)R⁵, —C(O)OR¹³, —OC(O)R¹¹, —OC(O)N(R¹¹)₂—C(O)N(R¹¹)₂, —N(R¹¹)C(O)R¹¹, —N(R¹¹)C(O)OR³, —N(R¹⁵)C(O)N(R¹⁵)₂, —N(R¹⁵)S(O)₂(R¹⁵), —S(O)R¹⁴, —S(O)₂R¹¹, —S(O)₂N(R¹¹)₂, —NO₂, and —CN; or
R³ and R⁴ can come together to form a 4- to 12-membered heterocycle optionally substituted with one or more substituents independently selected from:
halogen, —OR¹⁶, —SR¹⁶, —N(R⁶)₂, —C(O)R¹⁶, —C(O)OR⁶, —OC(O)R¹⁶, —OC(O)N(R¹⁶)₂, —C(O)N(R¹⁶)₂, —N(R¹⁶)C(O)R¹⁶, —N(R¹⁶)C(O)OR¹⁶, —N(R¹⁶)C(O)N(R¹⁶)₂, —N(R¹⁶)S(O)₂(R¹⁶), —S(O)R¹⁶, —S(O)₂R¹⁶, —S(O)₂N(R¹⁶)₂, —NO₂, ═O, and —CN; and
C₁₋₆ alkyl optionally substituted with one or more substituents independently selected from halogen, —OR¹⁶, —SR¹⁶, —N(R¹⁶)₂, —C(O)R¹⁶, —C(O)OR¹⁶, —OC(O)R¹⁶, —OC(O)N(R¹¹)₂, —C(O)N(R¹⁶)₂, —N(R¹¹)C(O)R¹⁶, —N(R¹⁶)C(O)OR¹², —N(R¹⁶)C(O)N(R¹⁶)₂, —N(R¹⁶)S(O)₂(R¹⁶), —S(O)R¹⁶, —S(O)₂R⁶, —S(O)₂N(R^{1G})₂, —NO₂, ═O, and —CN;
R⁵ is selected from:
halogen, —OR¹⁷, —SR¹⁷, —N(R¹⁷)₂, —C(O)R¹⁷, —C(O)OR¹⁷, —OC(O)R¹¹, —OC(O)N(R¹⁷)₂—C(O)N(R¹⁷)₂, —N(R¹)C(O)R¹, —N(R¹)C(O)OR¹, —N(R¹⁷)C(O)N(R¹⁷)₂, —N(R¹⁷)S(O)₂(R¹⁷), —S(O)R¹⁷, —S(O)₂R¹⁷, —S(O)₂N(R¹⁷)₂, —NO₂, and —CN;
C₁₋₆ alkyl and C₂₋₆ alkenyl each of which is optionally substituted with one or more substituents independently selected from halogen, —OR¹⁷, —SR¹⁷, —N(R¹⁷), —C(O)R¹⁷, —C(O)OR¹⁷, —OC(O)R¹¹, —OC(O)N(R¹⁷) —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)S(O)₂(R¹⁷), —S(O)R¹⁷, —S(O)₂R¹⁷, —S(O)₂N(R¹⁷)₂, —NO₂, ═O, and —CN; and
C₃₋₁₀ carbocycle optionally substituted with one or more substituents independently selected from halogen, —OR¹¹)_{7H}, —SR^{17B}, —N(R^{17B})₂, —C(O)R^{17B}, —C(O)OR^{17B}, —OC(O)R^{147B}, —OC(O)N(R^{17B}), —C(O)N(R¹⁴)₂, —N(R¹⁴)C(O)R¹⁴, —N(R¹⁷H)C(O)OR¹⁷B, —N(R¹⁷)C(O)N(R¹¹)₂, —N(R^{17B})S(O)₂(R^{17B}), —S(O)R⁷, —S(O)₂R¹⁷B, —S(O)₂N(R¹⁷)₂, —NO₂, and —CN;
R¹¹, R², R¹³, R¹⁴, R^{14H}, R¹⁵, R¹⁶, R¹¹, and R^{17B} are independently selected at each occurrence from (I), (II) and (III):
(I) hydrogen;
(II) C₁₋₆ alkyl optionally substituted with one or more substituents independently selected from: halogen, —OH, —O—C₁₋₆ alkyl, —O—C₁₋₆ haloalkyl —NH₂, —NO₂, ═O, —CN, C₃₋₁₀ carbocycle and 3- to 10-membered heterocycle, wherein the C₃₋₁₀ carbocycle and 3- to 10-membered heterocycle are optionally substituted with one or more substituents selected from: halogen, —OH, C₁-C₆ haloalkyl, —O—C₁-C₆ alkyl, —O—C₁-C₆haloalkyl —NH₂, —NO₂, =O, and —CN; and
(111) C₃₋₁₀ carbocycle and 3- to 10-membered heterocycle, each of which is optionally substituted with one or more substituents independently selected from: halogen, —OH, C₁-C₆ haloalkyl, —O—C₁-C₆ alkyl, —O—C₁-C₆haloalkyl —NH₂, —NO₂, ═O, and —CN; and C₁₋₆ alkyl optionally substituted with one or more substituents independently selected from halogen, —OH, —O—C₁-C₆ alkyl, —O—C₁-C₆ haloalkyl —NH₂, —NO₂, ═O, and —CN; and
n is selected from 0, 1, 2, 3, and 4,
wherein n is selected from 1, 2, 3, and 4; or
at least one of R¹ or R² is hydrogen.

2. The compound or salt of claim 1, wherein R⁵ is selected from C₃,-₀ carbocycle optionally substituted with one or more substituents independently selected from halogen, —OR¹¹)_{17B}, —SR¹⁷)_{17B}, —N(R¹¹)₂, —C(O)R¹⁶, —C(O)OR⁴B, —OC(O)R⁴”, —OC(O)N(R^{17B})₂, —C(O)N(R¹⁴¹)₂, —N(R^{17B})C(O)R¹⁷, —N(R^{17B})C(O)OR^{17B}, —N(R^{17B})C(O)N(R^{17B})₂, —N(R¹⁷)S(O)₂(RB), —S(O)R^{17B}, —S(O)₂R^{17B}, —S(O)₂N(R¹¹)_{B})₂, —NO₂, and —CN.

3. The compound or salt of claim 2, wherein R⁵ is selected from C_{3-f} carbocycle optionally substituted with one or more substituents independently selected from halogen, —OR¹⁶, —N(R^{14B})₂, —C(O)R¹¹)_{B}, —C(O)OR¹¹)_{B}, —NO₂, and —CN.

4. The compound or salt of claim 3, wherein R^{is selected from cyclopropyl, cyclobutyl, cyclopentyl, cyclohexyl, and phenyl, any one of which is optionally substituted.}

5. The compound or salt of claim 4, wherein R⁵ is represented by:

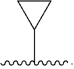 [SMILES: *C1CC1]

6. The compound or salt of any one of claims 1 to 5, wherein one of R¹ or R² is selected from:
C₁₋₆ alkyl optionally substituted with one or more substituents independently selected from 3- to 10-membered heterocycle optionally substituted with one or more substituents independently selected from halogen, —OR¹¹A, —SR^{14B}, —N(R^{14B})₂, —C(O)R^{14B}, —C(O)OR^{14B}, —OC(O)R^{14B}, —OC(O)N(R^{14B})₂, —C(O)N(R^{14B})₂, —N(R^{14B})C(O)R^{14B}, —N(R^{14B})C(O)OR¹⁴, —N(R^{14B})C(O)N(R^{14B})₂—N(R^{14B})S(Oh(R^{14B}), —S(O)R^{14B}, —S(O)₂R^{14B}, —S(O)₂N(R^{14B})₂, —NO₂, and —CN; and
3- to 10-membered heterocycle optionally substituted with one or more substituents independently selected from: halogen, —OR^{14B}, —SR^{14B}, —N(R^{14B})₂, —C(O)R¹⁴¹, —C(O)OR^{14B}, —OC(O)R^{14B}, —OC(O)N(R^{14B}), —C(O)N(R^{14B})₂, —N(R^{14B})C(O)R^{14B}, —N(R^{14B})C(O)OR^{14B}, —N(R^{14B})C(O)N(R^{14B})₂, —N(R^{14B})S(O)₂(R^{14B}), —S(O)R^{14B}—S(O)₂R^{14B}—S(O)₂N(R¹⁴)₂, —NO₂, and —CN.

7. The compound or salt of claim 6, wherein one of R¹ or R² is selected from: C₁₋₆ alkyl optionally substituted with one or more substituents independently selected from 5- to 6-membered heterocycle optionally substituted with one or more substituents independently selected from halogen, —OR^{14B}, —N(R^{14B})₂, —C(O)R^{14B}, —C(O)OR^{14B}, —NO₂, and —CN.

8. The compound or salt of claim 7, wherein one of R¹ or R² is C₁₋₆ alkyl substituted with a 5- to 6-membered saturated heterocycle wherein the 5- to 6-membered saturated heterocycle selected from pyrrolidine, pyrroline, pyrazoline, imidazoline, tetrahydrofuran, dioxolane, tetrahydrothiophene, piperidine, piperazine, tetrahydropyran, dioxane, thiane, dithiane, morpholine, and thiomorpholine, any one of which is optionally substituted.

9. The compound or salt of claim 8, wherein one of R¹ or R² is represented by:

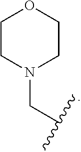 [SMILES: *CN1CCOCC1]

10. The compound or salt of claim 6, wherein one of R¹ or R² is a 5- to 6-membered heterocycle optionally substituted with one or more substituents independently selected from: halogen, —OR^{14B}, —N(R^{14B})₂, —C(O)R^{14B}, —C(O)OR^{14B}, —NO₂, and —CN.

11. The compound or salt of claim 10, wherein the 5- to 6-membered heterocycle of R¹ or R² is a 5- to 6-membered saturated heterocycle selected from pyrrolidine, pyrroline, pyrazoline, imidazoline, tetrahydrofuran, dioxolane, tetrahydrothiophene, piperidine, piperazine, tetrahydropyran, dioxane, thiane, dithiane, morpholine, and thiomorpholine, any one of which is optionally substituted.

12. The compound or salt of claim 11, wherein the 5- to 6-membered saturated heterocycle of R¹ or R² is represented by

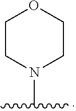 [SMILES: *N1CCOCC1]

13. The compound or salt of any one of claims 1 to 12, wherein one of R³ and R⁴ is selected from C₁ alkyl optionally substituted with one or more substituents independently selected from:
halogen, —OR¹¹, —SR¹¹, —N(R¹¹)₂, —C(O)R¹¹, —C(O)NH₂, —C(O)—O—C₁₋₆ alkyl, —OC(O)R¹¹, —OC(O)N(R¹¹)₂, —N(R¹¹)C(O)R¹², —N(R¹¹)C(O)OR¹¹, —N(R¹¹)C(O)N(R¹¹)₂, —N(Rts)S(O)₂(R¹¹), —S(O)R¹¹, —S(O)₂R¹¹, —S(O)₂N(R¹¹)₂, —NO₂, —CN;
C₃₋₁₀ carbocycle and 3- to 10-membered heterocycle any of which is optionally substituted with one or more substituents independently selected from halogen, C₁₋₆ haloalkyl, C₁₋₆ alkoxy, —OR¹¹, —SR¹¹, —N(R¹¹)₂, —C(O)R¹¹, —C(O)OR¹¹, —OC(O)R¹¹, —OC(O)N(R¹¹)₂, —C(O)N(R¹¹)₂, —N(R¹⁵)C(O)R¹⁵, —N(R¹⁵)C(O)OR¹⁵)₅, —N(R¹¹)C(O)N(R¹¹)₂, —N(R¹¹)S(O)₂(R¹¹), —S(O)R¹¹, —S(O)₂R¹¹)₅, —S(O)₂N(R¹¹)₂, —NO₂, and —CN.

14. The compound or salt of claim 13, wherein one of R³ and R¹ is selected from C₁ alkyl optionally substituted with one or more substituents independently selected from halogen, —OR¹¹, —N(R¹¹)₂, —C(O)R¹¹, —C(O)NH₂, —NO₂, and —CN.

15. The compound or salt of claim 14, wherein one of R³ and R¹ is represented by NH₂

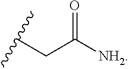 [SMILES: *CC(N)=O]

16. The compound or salt of claim 1, wherein the compound of Formula I is represented by Formula (I-a): 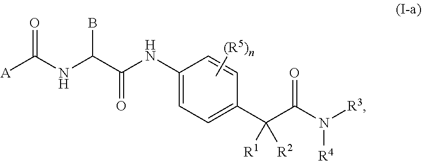
or a pharmaceutically acceptable salt thereof, wherein:
A is selected from a 5- to 6-membered heterocycle optionally substituted with one more substituents independently selected from (i), (ii), and (iii):
(i) halogen, —OR¹¹, —SR¹¹, —N(R¹¹)₂, —C(O)R¹¹, C(O)OR¹¹, —OC(O)R¹¹, —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹¹)₂, —N(R¹¹)S(Ob(R¹¹), —S(O)R¹¹, —S(O)₂R¹¹, —S(O)₂N(R¹¹)₂, —NO₂, and —CN;
(ii) C¹⁻¹⁰ alkyl optionally substituted with one or more substituents independently selected from:
halogen, —OR¹¹, —SR¹¹, —N(R¹¹)₂, —C(O)R¹¹, C(O)OR¹¹, —OC(O)R¹¹, —OC(O)N(R¹¹)₂, —C(O)N(R¹⁴)₂, —N(R¹⁴)C(O)R¹¹, —N(R¹¹)C(O)OR¹¹, —N(R¹¹)C(O)N(R¹¹)₂, —N(R¹¹)S(O)₂(R¹¹), —S(O)R¹¹, —S(O)₂R¹¹, —S(O)₂N(R¹¹)₂, —NO₂, ═O, —CN; and
C₃₋₁₀ carbocycle and 3- to 10-membered heterocycle each of which is optionally substituted with one or more substituents selected from: halogen, —OR¹¹, —SR¹¹, —N(R¹¹)ₗ)₂, —C(O)R¹¹, —C(O)OR¹¹, —OC(O)R¹¹, —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹¹)₂, —N(R¹¹)S(O)₂(R¹¹), —S(O)R¹¹, —S(O)₂R¹¹, —S(O)₂N(R¹¹)₂, —NO₂, ═O, and —CN; and
(iii) 3- to 10-membered heterocycle optionally substituted with one or more substituents independently selected from:
halogen, —OR¹¹, —SR¹¹, —N(R¹¹)₂, —C(O)R¹¹, —C(O)OR¹¹, —OC(O)R¹¹, —OC(O)N(R¹¹)₂—C(O)N(R¹¹)₂, —N(R^{1′})C(O)R¹¹, —N(R¹¹)C(O)OR¹¹, —N(R¹¹)C(O)N(R¹¹)₂, —N(R¹¹)S(O)₂(R¹), —S(O)R¹¹, —S(O)₂R¹¹, —S(O)₂N(R¹¹)₂, —NO₂, ═O, —CN; and
C₁₋₆ alkyl optionally substituted with one or more substituents independently selected from halogen, —OR¹¹, —SR¹¹, —N(R¹¹), —C(O)R¹¹, —C(O)OR¹¹, —OC(O)R¹¹, —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹¹)₂, —N(R¹¹)S(O)₂(R¹¹), —S(O)R, —S(O)₂R¹¹, —S(O)₂N(R¹¹)₂, —NO₂, ═O, and —CN;
B is selected from —CH(R^{A}) and C₃₋₁₀ carbocycle, wherein C_{1-3.1} carbocycle is optionally substituted with one or more substituents independently selected from:
halogen, —OR^{11B}, —SR^{11B}, —N(R¹²)₂, —C(O)R¹⁴, —C(O)OR¹¹, —OC(O)R¹¹)₂, —OC(O)N(R²)₂, —C(O)N(R¹⁾₂, —N(R¹²)C(O)R¹—N(R¹²)C(O)OR², —N(R¹²)C(O)N(R¹²)₂, —N(R¹²)S(O)₂(R¹²), —S(O)R¹², —S(O)₂R², —S(O)₂N(R²)₂, —NO₂, ═O, and —CN; and
C₁₋₆ alkyl optionally substituted with one or more substituents independently selected from halogen, —OR¹², —SR¹², —N(R¹¹)₄)₂, —C(O)R¹², —C(O)OR¹¹)₂, —OC(O)R¹⁴, —OC(O)N(R¹)z —C(O)N(R¹²)₂, —N(R¹¹)C(O)R¹², —N(R¹¹)₂)C(O)OR¹², —N(R¹²)C(O)N(R¹²)₂, —N(R¹²)S(O)₂(R¹²), —S(O)R^{1Z}, —S(O)₂R², —S(O)₂N(R¹²)₂, —NO₂, ═O, and —CN;
R^{A} and R^{B} are each independently selected from:
halogen, —OR¹¹, —SR¹¹, —N(R³)₂, —C(O)R¹³, —C(O)OR¹¹, —OC(O)R¹¹, —OC(O)N(R¹³)₂—C(O)N(R¹¹i)₂, —N(R¹³)C(O)R¹³, —N(R¹¹)C(O)OR³, —N(R¹¹)C(O)N(R¹¹)₂, —N(R¹¹)S(O)₂(R¹³), —S(O)R¹¹, —S(O)₂R¹³, —S(O)₂N(R¹¹)₂, —NO₂, and —CN;
C₁₋₆ alkyl optionally substituted with one or more substituents independently selected from halogen, —OR¹³, —SR¹³, —N(R¹¹)₂, —C(O)R¹¹, —C(O)OR¹¹, —OC(O)R¹¹, —OC(O)N(R¹¹)₂, —C(O)N(R¹³)₂,—N(R¹¹)C(O)R¹¹, —N(R³)C(O)OR¹¹, —N(R¹³)C(O)N(R¹³)₂,—N(R¹¹)₂S(O)₂(R¹³), —S(O)R¹³, —S(O)₂R³, —S(O)₂N(R¹³)₂, —NO₂, and —CN; and
C₃₋₁₀ carbocycle optionally substituted with one or more substituents independently selected from halogen, —OR¹³, —SR¹³, —N(R¹³)₂, —C(O)R¹¹, —C(O)OR¹¹, —OC(O)R¹³, —OC(O)N(R)₂, —C(O)N(R¹³)₂, —N(R¹³)C(O)R¹³, —N(R¹³)C(O)OR¹³, —N(R¹¹)C(O)N(R¹¹)₂, —N(R¹³)S(O)₂(R¹³), —S(O)R¹³, —S(O)₂R¹³, —S(O)₂N(R¹)₂, —NO₂, and —CN;
R¹ and R² are each independently selected from:
hydrogen;
halogen, —OR¹⁴, —SR¹⁴, —N(R¹⁴)₂, —C(O)R¹⁴, —C(O)OR¹⁴, —OC(O)R¹⁴, —OC(O)N(R¹⁴)₂, —C(O)N(R¹⁴)₂, —N(R¹⁴)C(O)R¹⁴, —N(R¹⁴)C(O)OR¹⁴, —N(R¹⁴)C(O)N(R¹⁴)₂, —N(R¹⁴)S(O)₂(R¹⁴), —S(O)R¹⁴, —S(O)₂R¹⁴, —S(O)₂N(R¹⁴)₂, —NO₂, and —CN; and
C₁₋₆ alkyl optionally substituted with one or more substituents independently selected from halogen, —OR¹⁴, —SR¹⁴, —N(R¹⁴)₂, —C(O)R¹⁴, —C(O)OR¹⁴, —OC(O)R¹⁴, —OC(O)N(R¹⁴)₂, —C(O)N(R¹⁴)₂, —N(R¹⁴)C(O)R¹⁴, —N(R¹⁴)C(O)OR¹⁴, —N(R¹⁴)C(O)N(R¹⁴)₂, —N(R¹⁴)S(O)₂(R¹⁴), —S(O)R¹⁴, —S(O)₂R¹⁴, —S(O)₂N(R¹⁴)₂, —NO₂, and —CN;
R³ and R⁴ are each independently selected from (a), (b), (c), and (d):
(a) hydrogen;
(b) C₁ alkyl optionally substituted with one or more substituents independently selected from:
halogen, —OR¹⁵, —SR¹⁵, —N(R¹⁵)₅)₂, —C(O)R¹², —C(O)—O—C₁₋₆ alkyl, —OC(O)R¹¹, —OC(O)N(R¹¹), —N(R¹⁵)C(O)R¹¹, —N(R¹⁵)C(O)OR¹⁹, —N(R¹¹)C(O)N(R³)₂, —N(R¹¹)₅)S(O)₂(R¹¹), —S(O)R¹¹, —S(O)₂R1-, —S(O)₂N(R¹¹)₂, —NO₂, —CN;
C₃₋₁₀ carbocycle and 3- to 10-membered heterocycle any of which is optionally substituted with one or more substituents independently selected from halogen, C₁₋₆haloalkyl, C₁₋₆ alkoxy, —OR¹⁴, —SR¹⁴, —N(R¹¹)₉)₂, —C(O)R¹¹, —C(O)OR¹¹, —OC(O)R^{11B}, —OC(O)N(R¹¹)₂, —C(O)N(R¹¹)₂, —N(R¹¹)C(O)R¹¹, —N(R¹¹)C(O)OR¹¹, —N(R¹⁴)C(O)N(R¹¹), —N(R¹⁴)S(O)₂(R¹⁵), —S(O)R¹¹, —S(O)₂R¹¹, —S(O)₂N(R¹⁵)₂, —NO₂, and —CN;
(c) C₂₋₆ alkyl optionally substituted with one or more substituents independently selected from:
halogen, —OR¹¹, —SR¹⁵, —N(R¹¹)₂, —C(O)R, —C(O)—O—C₁,&,alkyl, —OC(O)R¹¹, —OC(O)N(R¹¹)₂, —C(O)N(R⁵)₂, —N(R¹⁴)C(O)R¹¹, —N(R¹⁴)C(O)OR¹¹, —N(R¹¹)₅)C(O)N(R¹¹)₅)₂, —N(R^{1S})S(O)₂(R¹¹)₅), —S(O)R¹¹, —S(O)₂R^{1S}, —S(O)₂N(R¹¹)₂, —NO₂, and —CN;
C₃₋₁₀ carbocycle and 3- to 10-membered heterocycle any of which is optionally substituted with one or more substituents independently selected from halogen, C₁₋₆ haloalkyl, C₁₋₆ alkoxy, —OR¹¹, —SR¹⁵, —N(R¹¹)₂, —C(O)R¹¹, —C(O)OR¹³, —OC(O)R¹¹, —OC(O)N(R¹¹)₂, —C(O)N(R¹¹)₂, —N(R¹¹)C(O)R¹⁴, —N(R⁵)C(O)OR¹¹, —N(R¹⁵)C(O)N(R¹⁵)₂, —N(R¹⁵)S(O)₂(R¹⁵), —S(O)R¹⁵, —S(O)₂R^{1S}, —S(O)₂N(R¹⁵), —NO₂, and —CN; and
(d) C₃₋₁₀ carbocycle and 3- to 10-membered heterocycle any of which is optionally substituted with one or more substituents independently selected from:
halogen, —OR¹⁵, —SR¹⁵, —N(R¹⁵)₂, —C(O)R¹⁵, —C(O)OR¹⁵, —OC(O)R¹⁵, —OC(O)N(R¹⁵)₂—C(O)N(R¹⁵)₂, —N(R¹⁵)C(O)R¹⁵, —N(R¹⁵)C(O)OR¹⁵, —N(R¹⁵)C(O)N(R¹⁵)₂, —N(R¹⁵)S(O)₂(R¹⁵), —S(O)R¹⁵)₅, —S(O)₂R¹⁵, —S(O)₂N(R¹⁵)₅)₂, —NO₂, and —CN; and
C₁₋₆ alkyl optionally substituted with one or more substituents independently selected from halogen, —OR¹⁵, —SR¹⁵, —N(R¹⁵)₂, —C(O)R¹⁵, —C(O)OR¹³, —OC(O)R¹⁵, —OC(O)N(R¹⁵)₂, —C(O)N(R¹⁵)₂, —N(R¹⁵)C(O)R¹⁵, —N(R⁵)C(O)OR¹⁵, —N(R¹⁵)C(O)N(R¹⁴)₂, —N(R¹⁴)S(O)₂(R¹⁵), —S(O)R¹⁵, —S(O)₂R¹⁵, —S(O)₂N(R¹⁵)₂, —NO₂, and —CN; or
R³ and R⁴ can come together to form a 4- to 12-membered heterocycle optionally substituted with one or more substituents independently selected from:
halogen, —OR¹⁶, —SR^{M}, —N(R¹⁶)₂, —C(O)R¹⁵, —C(O)OR¹⁶, —OC(O)R¹⁶, —OC(O)N(R¹⁶)₂—C(O)N(R¹⁶)₂, —N(R¹⁶)C(O)R¹⁶, —N(R¹⁶)C(O)OR¹⁶, —N(R¹⁶)C(O)N(R¹⁶)₂, —N(R¹⁶)S(O)₂(R¹⁶), —S(O)R¹⁶, —S(O)₂R⁶, —S(O)₂N(R⁶)₂, —NO₂, ═O, and —CN; and
C₁₋₆ alkyl optionally substituted with one or more substituents independently selected from halogen, —OR¹⁶, —SR¹⁶, —N(R¹⁶)₂, —C(O)R¹⁶, —C(O)OR¹⁶, —OC(O)R¹⁶, —OC(O)N(R¹⁶)₂, —C(O)N(R¹⁶)₂, —N(R¹)C(O)R¹⁶, —N(R¹⁶)C(O)OR¹², —N(R¹⁶)C(O)N(R¹⁶)₂, —N(R¹⁶)S(O)₂(R¹⁶), —S(O)R¹⁶, —S(O)₂R¹⁶, —S(O)₂N(R¹⁶)₂, —NO₂, =O, and —CN;
R⁵ is selected from:
halogen, —OR¹⁷, —SR¹⁷, —N(R¹⁷)₂, —C(O)R¹⁷, —C(O)OR¹⁷, —OC(O)R¹¹, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂—N(R¹⁷)S(O)₂(R¹⁷), —S(O)R¹⁷, —S(O)₂R¹⁷, —S(O)₂N(R¹⁷)₂, —NO₂, and —CN; and
C₁₋₆ alkyl and C₂₋₆ alkenyl each of which is optionally substituted with one or more substituents independently selected from halogen, —OR¹⁷, —SR¹⁷, —N(R¹⁷)₂, —C(O)R¹⁷, —C(O)OR¹⁷, —OC(O)R¹¹, —OC(O)N(R¹⁷), —C(O)N(R¹⁷)₂, —N(R¹⁷)C(O)R¹⁷, —N(R¹⁷)C(O)OR¹⁷, —N(R¹¹)₇)C(O)N(R¹¹)₂, —N(R¹⁷)S(O)₂(R¹⁷), —S(O)R¹⁷, —S(O)₂R¹⁷, —S(O)₂N(R¹⁷)₂, —NO₂, ═O, and —CN;
R¹¹, R¹², R¹³, R¹⁴, R¹⁵, R¹⁶, and R¹⁷ are independently selected at each occurrence from (I), (II) and (III):
(I) hydrogen,;
(II) C₁₋₆ alkyl optionally substituted with one or more substituents independently selected from: halogen, —OH, —O—C₁-C₆ alkyl, —O—C₁-C₆haloalkyl —NH₂, —NO₂, ═O, —CN, C₃₋₁₀ carbocycle and 3- to 10-membered heterocycle, wherein the C₃₋₁₀ carbocycle and 3- to 10-membered heterocycle are optionally substituted with one or more substituents selected from: halogen, —OH, C₁-C₆ haloalkyl, —O—C₁-C₆ alkyl, —O—C₁-C₆haloalkyl —NH₂, —NO₂, ═O, and —CN; and
(III) C₃₋₁₀ carbocycle and 3- to 10-membered heterocycle, each of which is optionally substituted with one or more substituents independently selected from: halogen, —OH, C₁-C₆ haloalkyl, —O—C₁-C₆ alkyl, —O—C₁-C₆haloalkyl —NH₂, —NO₂, ═O, and —CN; and C₁₋₆ alkyl optionally substituted with one or more substituents independently selected from halogen, —OH, —O—C₁-C₆ alkyl, —O—C₁-C₆haloalkyl —NH₂, —NO₂, ═O, and —CN; and
n is selected from 0, 1, 2, 3, and 4,
wherein n is selected from 1, 2, 3, and 4; or
at least one of R¹ or R² is hydrogen.

17. The compound or salt of claim 16, wherein n is selected from 1, 2, 3, and 4.

18. The compound or salt of claim 16 or claim 17, wherein n is selected from 1 and 2.

19. The compound or salt of any one of claims 16 to 18, wherein n is 1.

20. The compound or salt of any one of claims 16 to 19, wherein R is selected from halogen, —OR¹⁷, —SR¹⁷, —N(R¹⁷)₂, —C(O)R¹⁷, —NO₂, and —CN; and
C₁₋₃ alkyl and C₂₋₃ alkenyl each of which is optionally substituted with one or more substituents independently selected from halogen, —OR¹⁷, —SR,¹⁷—N(R¹⁷)₂, —C(O)R¹², —NO₂, ═O, and —CN.

21. The compound or salt of any one of claims 16 to 20, wherein R⁵ is selected from chloro, fluoro, bromo, —OR¹⁷, —N(R¹⁷)₂, —CN;
C₁₋₃ alkyl and C₂0.3 alkenyl each of which is optionally substituted with one or more substituents independently selected from chloro, fluoro, —OR¹⁷, and —N(R¹¹).

22. The compound or salt of claim 21, wherein R³ is selected from chloro, fluoro, bromo, —CN, methyl, ethyl,

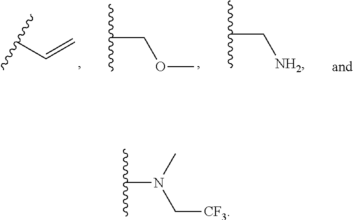

23. The compound or salt of claim 21, wherein R⁵ is selected from chloro, fluoro, bromo, —CN, methyl, ethyl,

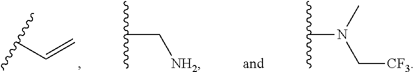

24. The compound or salt of any of claims 16 to 23, wherein A is selected from 5- to 6-membered heteroaryl optionally substituted with one or more substituents independently selected from:
C₁₋₁₀ alkyl optionally substituted with one or more substituents independently selected from:
halogen, —OR¹¹, —N(R¹¹)₂, —C(O)R¹¹, —C(O)OR¹¹, —NO₂, ═O, —CN; and C₃₋₁₀ carbocycle and 3- to 10-membered heterocycle each of which is optionally substituted with one or more substituents selected from halogen, —OR¹¹, —N(R¹¹)ₗ)₂, —C(O)R¹¹, —C(O)OR¹¹, —NO₂, ═O, —CN; and
3- to 10 membered heterocycle optionally substituted with one or more substituents independently selected from:
halogen, —OR¹¹, —N(R¹¹)₂, —C(O)R¹¹, —C(O)OR¹¹, —NO₂, ═O, —CN; and C₁₋₆ alkyl optionally substituted with one or more substituents independently selected from halogen, —OR¹¹, —N(R¹¹)₂, —C(O)R¹¹, —C(O)OR¹¹, —NO₂, =O, and —CN.

25. The compound or salt of any one of claims 16 to 24, wherein A is selected from an optionally substituted 5-membered heteroaryl.

26. The compound or salt of claim 25, wherein A is selected from pyrazole, oxazole, isoxazole, thiazole, isothiazole, pyrrole, furan, thiophene, imidazole, triazole, tetrazole, and pyridine, any of which is optionally substituted.

27. The compound or salt of claim 26, wherein A is selected from optionally substituted pyrazole and optionally substituted isoxazole.

28. The compound or salt of claim 27, wherein A is selected from: 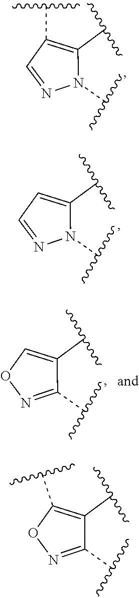
each of which is optionally substituted and wherein 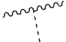
denotes the connection to the optional substituent and 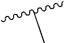
denotes the connection of A to the remainder of Formula I.

29. The compound or salt of claim 25, wherein the 5- to 6-membered heterocycle of A is substituted with one or more substituents.

30. The compound or salt of claim 29, wherein the substituents on the 5- to 6-membered heterocycle of A are independently selected from (i), (ii), and (iii):
(i) halogen, —OR¹¹, —SR¹¹, —N(R¹¹)₂, —C(O)R¹¹, —NO₂, ═O, and —CN;
(ii) C₁₋₆ alkyl optionally substituted with one or more substituents independently selected from:
halogen, —OR¹¹, —SR¹¹, —N(R¹¹)₂, —C(O)R¹¹, —NO₂, ═O, —CN; and
C₃₋₁₀ carbocycle and 3- to 10-membered heterocycle each of which is optionally substituted with one or more substituents selected from: halogen, —OR¹¹, —SR¹¹, —N(R¹¹)₂, —C(O)R¹¹, —NO₂, ═O, and —CN; and
(iii) 3- to 10-membered heterocycle optionally substituted with one or more substituents independently selected from:
halogen, —OR¹¹, —SR¹¹, —N(R¹¹)₂, —C(O)R¹¹, —NO₂, ═O, —CN; and C₁₋₆ alkyl optionally substituted with halogen.

31. The compound or salt of claim 29 or claim 30, wherein the substituents on the 5- to 6-membered heterocycle of A are independently selected from: fluorine, methyl, ethyl, isopropyl,

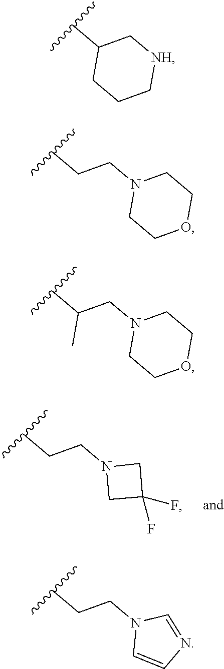

32. The compound or salt of claim 29 or claim 30, wherein A is selected from:

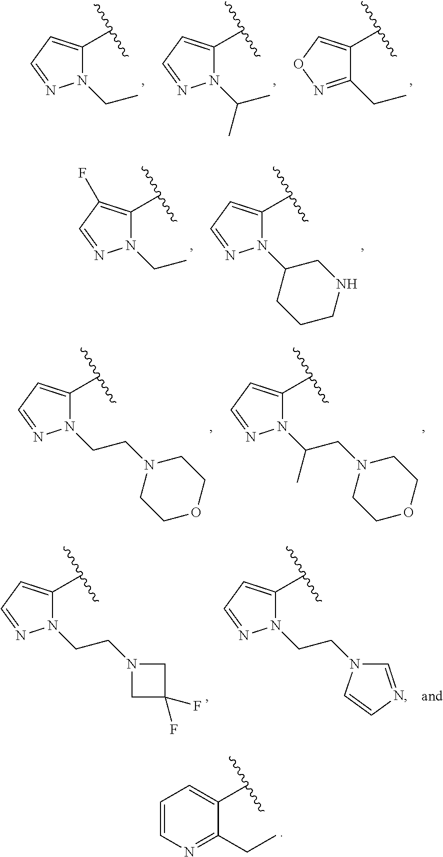

33. The compound or salt of any one of claims 16 to 32, wherein B is selected from —CH(R^{A})(R^{B}) and optionally substituted C₆₋₁₀ carbocycle.

34. The compound or salt of claim 33, wherein B is —CH(R^{A})(R^{B}) and R^{A} and R^{B} are independently selected from:
C₁₋₆ alkyl optionally substituted with one or more substituents independently selected from halogen, —OR¹³, —SR¹¹, —N(R¹¹)₂, —C(O)R¹¹, —NO₂, and —CN; and
C₃₋₆ carbocycle optionally substituted with one or more substituents independently selected from halogen, —OR¹³, —SR¹³, —N(R¹³,)₂, —C(O)R¹², —NO₂, and —CN.

35. The compound or salt of any one of claims 16 to 34, wherein R^{A} and R^{B} are independently selected from: ethyl, propyl, isopropyl, cyclopropyl, cyclobutyl, cyclopentyl, and phenyl, any of which is optionally substituted.

36. The compound or salt of claim 33, wherein B is an optionally substituted Cs-s cycloalkyl or optionally substituted C₇₋₁₀ bicyclic carbocycle.

37. The compound or salt of claim 36, wherein B is an optionally substituted C₆,jo carbocycle selected from cyclohexyl, cycloheptyl, cyclooctyl, spiro [2.5] octanyl, and indanyl any of which are optionally substituted.

38. The compound or salt of claim 37, wherein B is represented by:

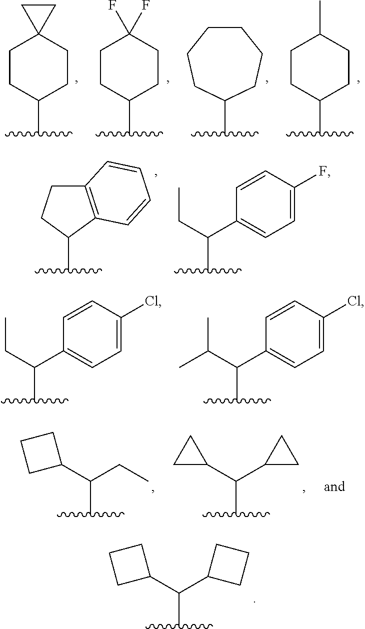

39. The compound or salt of any one of claims 16 to 38 wherein R¹ and R² are each independently selected from:
hydrogen, halogen, —OR¹⁴, —NO₂, and —CN; and
C₁₋₆ alkyl optionally substituted with one or more substituents independently selected from halogen, —OR¹⁴, —SR¹⁴, —N(R¹⁴)₂, —C(O)R¹⁴, —C(O)OR¹⁴, —OC(O)Ra, —OC(O)N(R¹⁴)₂, —C(O)N(R¹⁴)₂, —N(R¹⁴)C(O)R¹⁴, —N(R¹⁴)C(O)OR¹⁴, —N(R¹⁴)C(O)N(R¹⁴)₂, —N(R¹⁴)S(O)₂(R¹⁴), —S(O)R¹⁴, —S(O)₂R¹⁴, —S(O)₂N(R¹⁴)₂, —NO₂, and —CN.

40. The compound or salt of claim 39, wherein R¹ is hydrogen; and R² is selected from: methyl, ethyl, propyl, isopropyl and butyl, each of which is optionally substituted with one or more substituents independently selected from chloro, fluoro, bromo, and —OR¹⁴.

41. The compound or salt of claim 40, wherein R¹ and R² are each independently selected from: hydrogen,

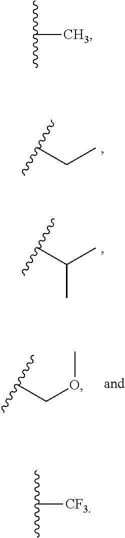

42. The compound or salt of claim 39, wherein R¹ is hydrogen; and R² is selected from: methyl, ethyl, propyl, and butyl, each of which is optionally substituted with one or more substituents independently selected from chloro, fluoro, bromo, and —OR¹⁴.

43. The compound or t of claim 39 wherein R¹ and R² are each independently selected from: hydrogen,

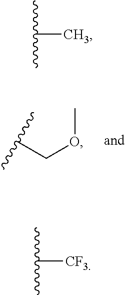

44. The compound or salt of any one of claims 16 to 43, wherein R³ and R¹ are each independently selected from:
(a) hydrogen;
(b) C₁ alkyl optionally substituted with one or more substituents independently selected from halogen, —OR¹⁵, —N(R¹⁵)₂, —C(O)R¹¹, —NO₂, —CN; and C₃₋₆ carbocycle optionally substituted with one or more substituents independently selected from halogen, C₁₋₆haloalkyl, —OR¹¹, —N(R¹¹)₂, —C(O)R¹¹, —NO₂, and —CN;
(c) C₂₋₆ alkyl optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —N(R¹⁵)C(O)OR¹⁵, —NO₂, —CN; carbocycle and 3- to 6-membered heterocycle any of which is optionally substituted with one or more substituents independently selected from halogen, —OR¹¹, —SR¹⁴, —N(R¹¹)₂, —C(O)R¹¹, —NO₂, and —CN; and
(d) C₃₋₆ carbocycle and 3- to 6-membered heterocycle each of which is optionally substituted with one or more substituents independently selected from halogen, —OR¹⁴, —SR¹¹, —N(R¹¹)₂, —C(O)R¹¹, —NO₂, —CN; and C₁₋₆ alkyl optionally substituted with one or more substituents independently selected from halogen, —OR¹¹, —SR¹¹, —N(R¹¹)₂, —C(O)R¹¹, —NO₂, and —CN.

45. The compound or salt of claim 44, wherein R³ or R¹⁴ is selected from C₁ alkyl optionally substituted with one or more substituents independently selected from halogen, —OR¹⁵; and cyclopropyl, cyclobutyl, cyclopentyl, cyclohexyl, and phenyl, each of which is optionally substituted with one or more substituents independently selected from halogen, C₁₋₃ haloalkyl and —OR¹⁵.

46. The compound or salt of claim 45, wherein R³ or R¹⁴ is selected from methyl,

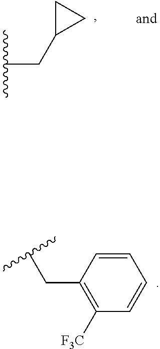 [SMILES: *CC1CC1 ; *Cc1ccccc1C(F)(F)F]

47. The compound or salt of claim 44, wherein R³ or R¹⁴ is selected from C₂₋₆ alkyl optionally substituted with one or more substituents independently selected from halogen, —OR¹¹, —SR¹¹, —N(R¹⁵)₂, —C(O)R¹⁵, —C(O)N(R¹⁵)₂, —N(R¹¹)C(O)OR¹¹, —NO₂, —CN; C₃₋₆ carbocycle and 3- to 6-membered heterocycle any of which is optionally substituted with one or more substituents independently selected from halogen, —OR¹¹, —SR¹¹, —N(R¹¹)₂, —C(O)R¹¹, —NO₂, and —CN.

48. The compound or salt of claim 47, wherein R³ or R¹¹s selected from ethyl, propyl, isopropyl, isobutyl, and butyl, each of which is optionally substituted with one or more substituents independently selected from chloro, fluoro, bromo, —OR¹², —N(R¹⁵)₂, —C(O)N(R¹¹)₂ and —N(R¹¹)C(O)OR¹⁵.

49. The compound or salt of claim 47 or claim 48, wherein R³ or R⁴ is selected from

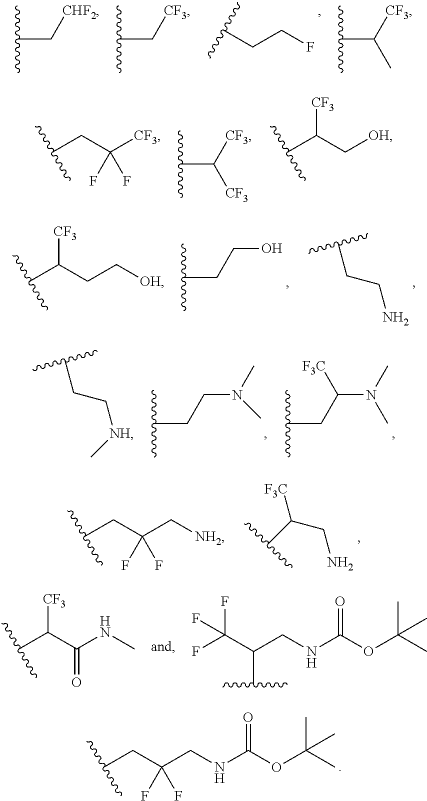

50. The compound or salt of claim 47 or claim 48, wherein R³ or R¹⁴ is selected from

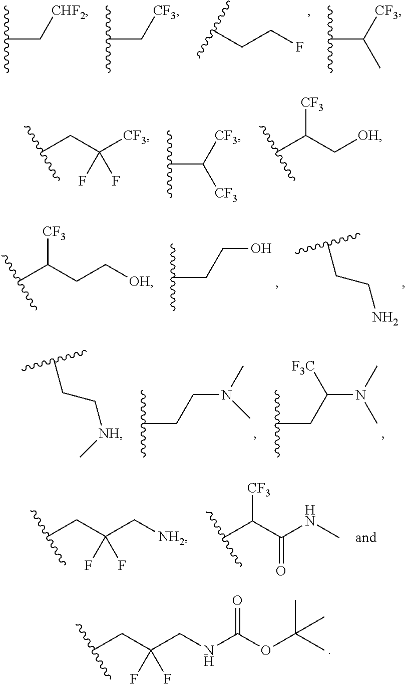

51. The compound or salt of claim 44, wherein R³ or R⁴ is selected from C₂₋₆ alkyl optionally substituted with one or more substituents independently selected from halogen, —OR¹¹, —N(R¹¹)₂, C₃-₆ carbocycle and 3- to 6-membered heterocycle any of which is optionally substituted with one or more substituents independently selected from halogen, —OR¹¹, —SR¹⁴, —N(R¹¹)₂, —C(O)R¹¹, —NO₂, and —CN.

52. The compound or salt of claim 51, wherein R³ or R¹⁴ is selected from

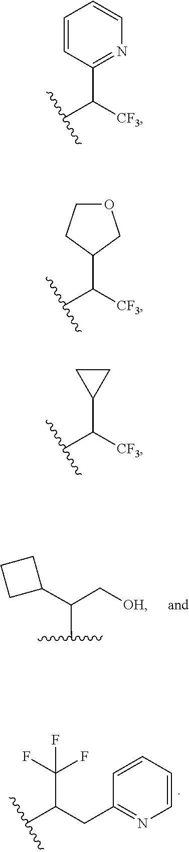 [SMILES: *C(c1ccccn1)C(F)(F)F ; *C(C1CCOC1)C(F)(F)F ; *C(C1CC1)C(F)(F)F ; *C(CO)C1CCC1 ; *C(Cc1ccccn1)C(F)(F)F]

53. The compound or salt of claim 51, wherein R³ or R¹⁴ is selected from

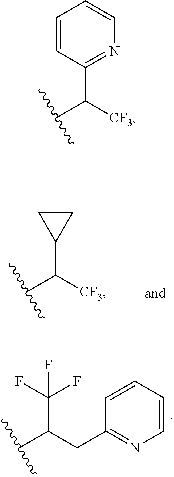 [SMILES: *C(c1ccccn1)C(F)(F)F ; *C(C1CC1)C(F)(F)F ; *C(Cc1ccccn1)C(F)(F)F]

54. The compound or salt of claim 44, wherein R³ or R¹⁴ is selected from cyclopropyl, cyclobutyl, cyclopentyl, cyclohexyl, phenyl, oxetanyl, azetidine, pyrrolidinyl, and pyridinyl, each of which is optionally substituted with one or more substituents independently selected from chloro, fluoro, bromo, —OR¹⁵, —NO₂, —CN; and C₁₋₃ alkyl optionally substituted with one or more substituents independently selected from chloro, fluoro, bromo, —OR¹⁴, —NO₂, and —CN.

55. The compound or salt of claim 54, wherein R³ or R¹⁴ is selected from

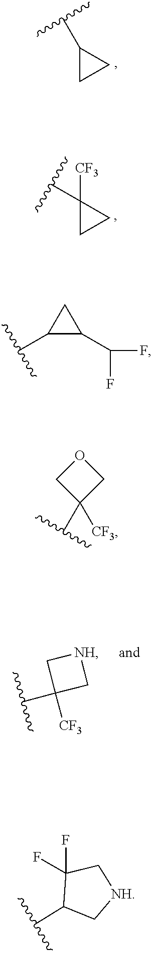

56. The compound or salt of any one claims 16 to 43, wherein R³ and R⁴ of 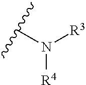
come together to form a 4- to 12-membered heterocycle any one of which is optionally substituted with one or more substituents independently selected from:
halogen, —OR¹⁶, —SR¹⁶, —N(R¹⁶)₂, —C(O)R¹⁶, —C(O)OR¹⁶, —OC(O)R¹⁶, —OC(O)N(R¹⁶)₂, —C(O)N(R¹⁶)₂, —N(R¹⁶)C(O)R¹⁶, —N(R¹⁶)C(O)OR¹⁶, —N(R¹G)C(O)N(R¹⁶)₂, —NO₂, ═O, and —CN; and
C₁₋₆ alkyl optionally substituted with one or more substituents independently selected from halogen, —OR⁶, —SR⁶, —N(R¹⁶)₂, —C(O)R¹⁶, —NO₂, and —CN.

57. The compound or salt of claim 56, wherein the optionally substituted 4- to 12-membered heterocycle optionally comprises more than one heteroatom selected from oxygen, nitrogen, sulfur, and any combination thereof.

58. The compound or salt of claim 57, wherein the R³ and R⁴ of 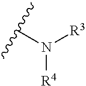
come together to form a saturated 4- to 8-membered heterocycle any one of which is optionally substituted.

59. The compound or salt of claim 58, wherein R³ and R⁴ come together to form a saturated 4- to 8-membered heterocycle and the saturated 4- to 8-membered heterocycle is selected from azetidine, oxetane, pyrrolidine, pyrazolidine, imidazolidine, oxazolidine, tetrahydrofuran, tetrahydrothiophene, piperidine, piperazine, tetrahydropyran, morpholine, oxazepane, and azaspiro[3.3]heptane, any one of which is optionally substituted with one substituents independently selected from chloro, fluoro, —OR¹⁶, —N(R¹⁶)₂, —C(O)OR¹⁶, —C(O)N(R¹⁶)₂, —N(R¹⁶)C(O)R¹⁶, —NO₂, and —CN; and C₁₋₃ alkyl optionally substituted with one or more substituents independently selected from chloro, fluoro, OR¹⁶, —NO₂, and —CN.

60. The compound or salt of claim 59, wherein R³ and R⁴ of 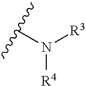
come together to form the saturated 4- to 8-membered heterocycle and the 4- to 8-membered heterocycle is selected from:

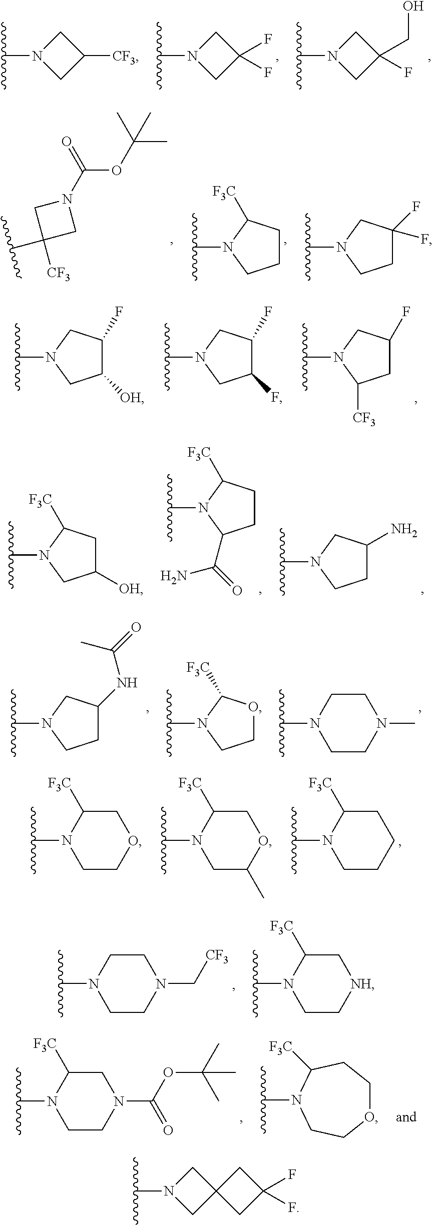

61. The compound or salt of claim 58, wherein the saturated 4- to 8-membered heterocycle is selected from azetidine, oxetane, pyrrolidine, pyrazolidine, imidazolidine, oxazolidine, tetrahydrofuran, tetrahydrothiophene, piperidine, piperazine, tetrahydropyran, morpholine, oxazepane, and azaspiro[3.3]heptane, any one of which is optionally substituted with one substituents independently selected from chloro, fluoro, —OR¹⁶, —NO₂, —C(O)N(R¹⁶)₂, and —CN; and C₁₋₃ alkyl optionally substituted with one or more substituents independently selected from chloro, fluoro, OR¹⁶, —NO₂, and —CN.

62. The compound or salt of claim 61, wherein R³ and R⁴ of 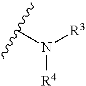
come together to form the saturated 4- to 8-membered heterocycle and the 4- to 8-membered heterocycle is selected from:

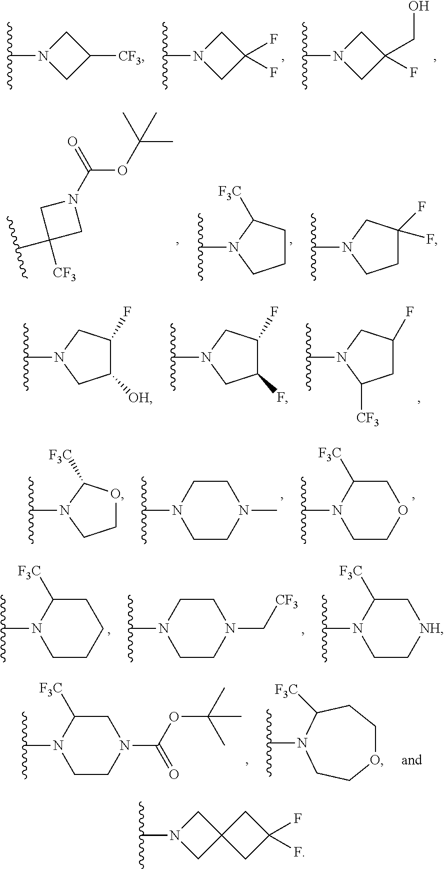

63. The compound or salt of claim 56, wherein the 4- to 12-membered heterocycle is an unsaturated 4- to 12-membered heterocycle any one of which is optionally substituted.

64. The compound or salt of claim 63, wherein the unsaturated 4- to 12-membered heterocycle is represented by

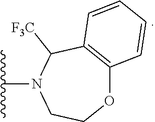 [SMILES: *N1CCOc2ccccc2C1C(F)(F)F]

65. A compound selected from: 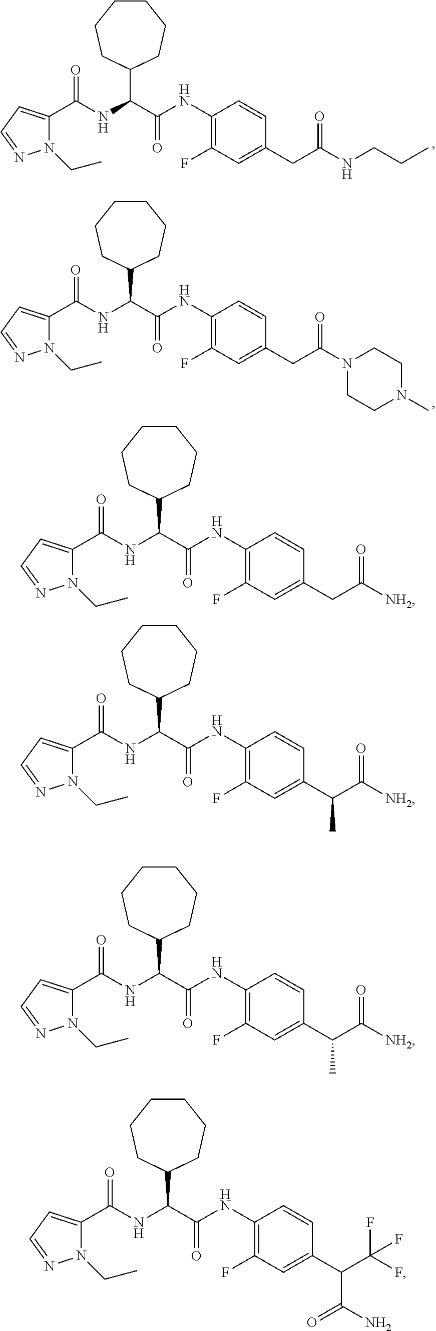 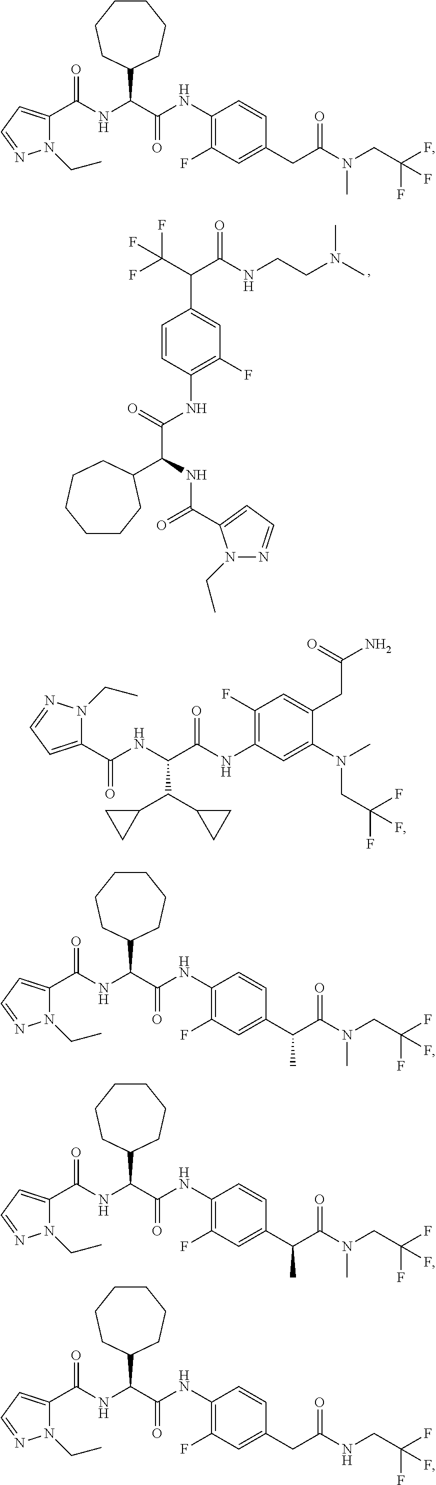 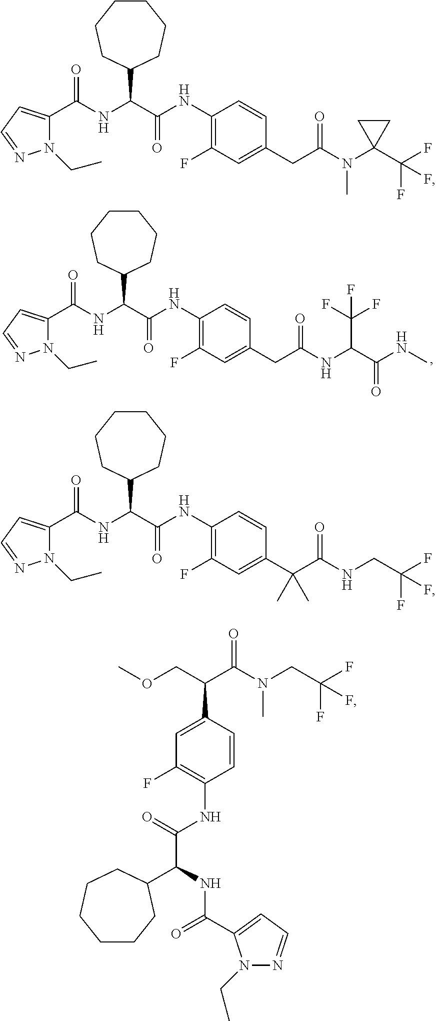 [SMILES: CCn1nccc1C(=O)N[C@H](C(=O)Nc1ccc(CC(=O)N(C)C2(C(F)(F)F)CC2)cc1F)C1CCCCCC1 ; CCn1nccc1C(=O)N[C@H](C(=O)Nc1ccc(CC(=O)NC(C(=O)NC)C(F)(F)F)cc1F)C1CCCCCC1 ; CCn1nccc1C(=O)N[C@H](C(=O)Nc1ccc(C(C)(C)C(=O)NCC(F)(F)F)cc1F)C1CCCCCC1 ; CCn1nccc1C(=O)N[C@H](C(=O)Nc1ccc([C@@H](COC)C(=O)N(C)CC(F)(F)F)cc1F)C1CCCCCC1] 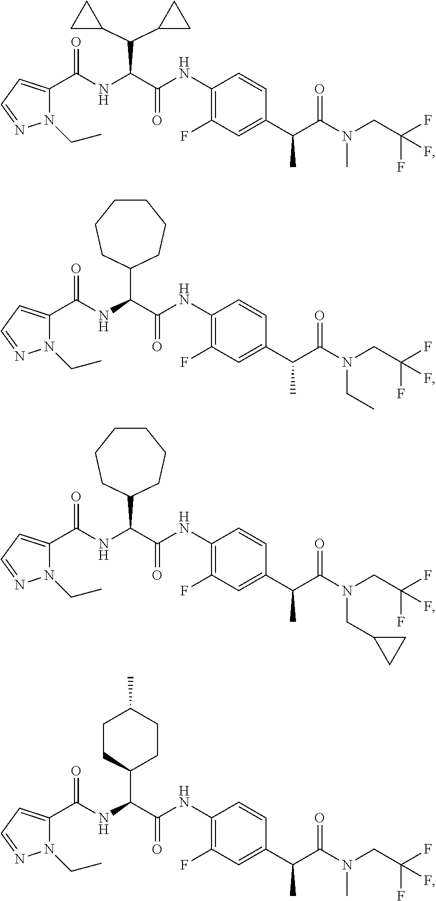 [SMILES: CCn1nccc1C(=O)N[C@H](C(=O)Nc1ccc([C@H](C)C(=O)N(C)CC(F)(F)F)cc1F)C(C1CC1)C1CC1 ; CCN(CC(F)(F)F)C(=O)[C@H](C)c1ccc(NC(=O)[C@@H](NC(=O)c2ccnn2CC)C2CCCCCC2)c(F)c1 ; CCn1nccc1C(=O)N[C@H](C(=O)Nc1ccc([C@H](C)C(=O)N(CC2CC2)CC(F)(F)F)cc1F)C1CCCCCC1 ; CCn1nccc1C(=O)N[C@H](C(=O)Nc1ccc([C@H](C)C(=O)N(C)CC(F)(F)F)cc1F)[C@H]1CC[C@H](C)CC1] 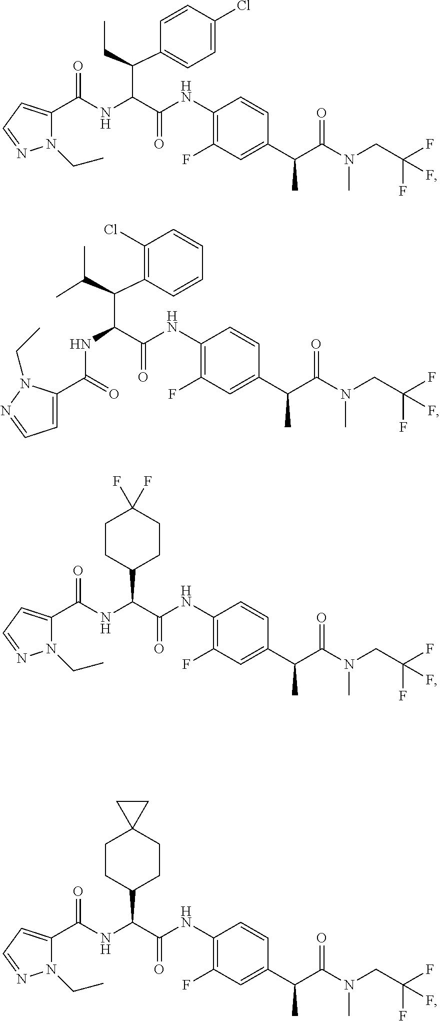 [SMILES: CC[C@@H](c1ccc(Cl)cc1)C(NC(=O)c1ccnn1CC)C(=O)Nc1ccc([C@H](C)C(=O)N(C)CC(F)(F)F)cc1F ; CCn1nccc1C(=O)N[C@H](C(=O)Nc1ccc([C@H](C)C(=O)N(C)CC(F)(F)F)cc1F)[C@H](c1ccccc1Cl)C(C)C ; CCn1nccc1C(=O)N[C@H](C(=O)Nc1ccc([C@H](C)C(=O)N(C)CC(F)(F)F)cc1F)C1CCC(F)(F)CC1 ; CCn1nccc1C(=O)N[C@H](C(=O)Nc1ccc([C@H](C)C(=O)N(C)CC(F)(F)F)cc1F)C1CCC2(CC1)CC2] 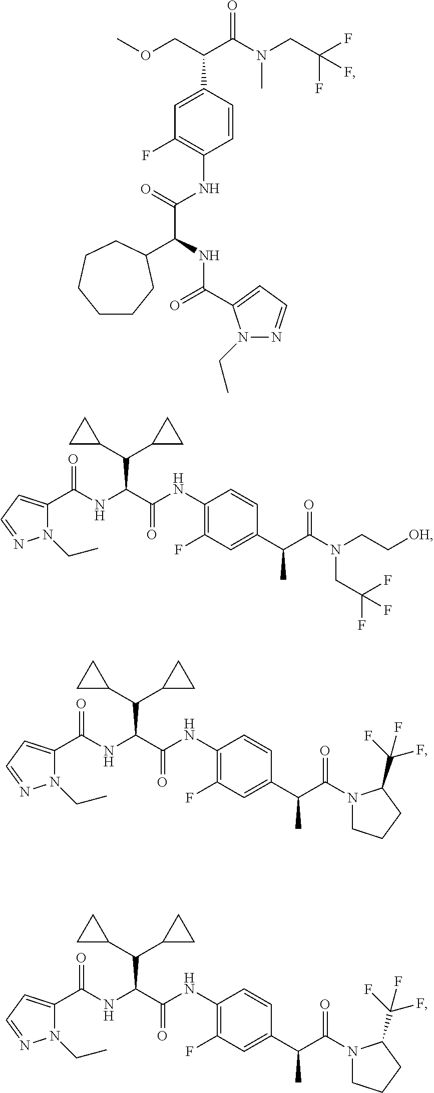 [SMILES: CCn1nccc1C(=O)N[C@H](C(=O)Nc1ccc([C@H](COC)C(=O)N(C)CC(F)(F)F)cc1F)C1CCCCCC1 ; CCn1nccc1C(=O)N[C@H](C(=O)Nc1ccc([C@H](C)C(=O)N(CCO)CC(F)(F)F)cc1F)C(C1CC1)C1CC1 ; CCn1nccc1C(=O)N[C@H](C(=O)Nc1ccc([C@H](C)C(=O)N2CCC[C@@H]2C(F)(F)F)cc1F)C(C1CC1)C1CC1 ; CCn1nccc1C(=O)N[C@H](C(=O)Nc1ccc([C@H](C)C(=O)N2CCC[C@H]2C(F)(F)F)cc1F)C(C1CC1)C1CC1] 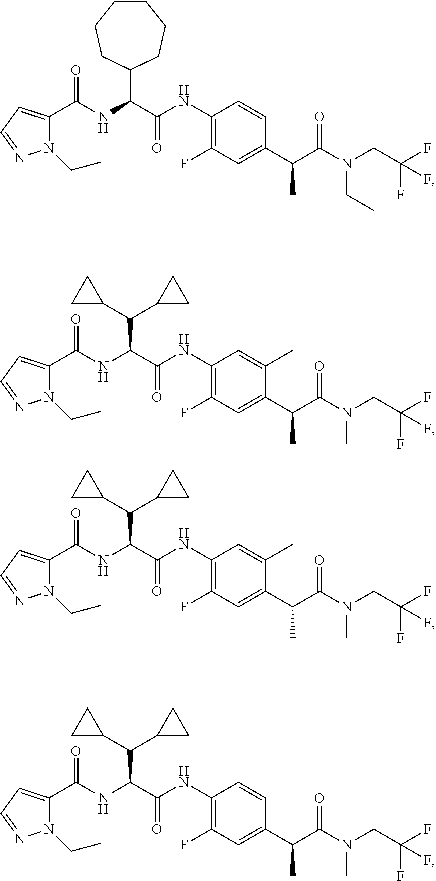 [SMILES: CCN(CC(F)(F)F)C(=O)[C@@H](C)c1ccc(NC(=O)[C@@H](NC(=O)c2ccnn2CC)C2CCCCCC2)c(F)c1 ; CCn1nccc1C(=O)N[C@H](C(=O)Nc1cc(C)c([C@H](C)C(=O)N(C)CC(F)(F)F)cc1F)C(C1CC1)C1CC1 ; CCn1nccc1C(=O)N[C@H](C(=O)Nc1cc(C)c([C@@H](C)C(=O)N(C)CC(F)(F)F)cc1F)C(C1CC1)C1CC1 ; CCn1nccc1C(=O)N[C@H](C(=O)Nc1ccc([C@H](C)C(=O)N(C)CC(F)(F)F)cc1F)C(C1CC1)C1CC1] 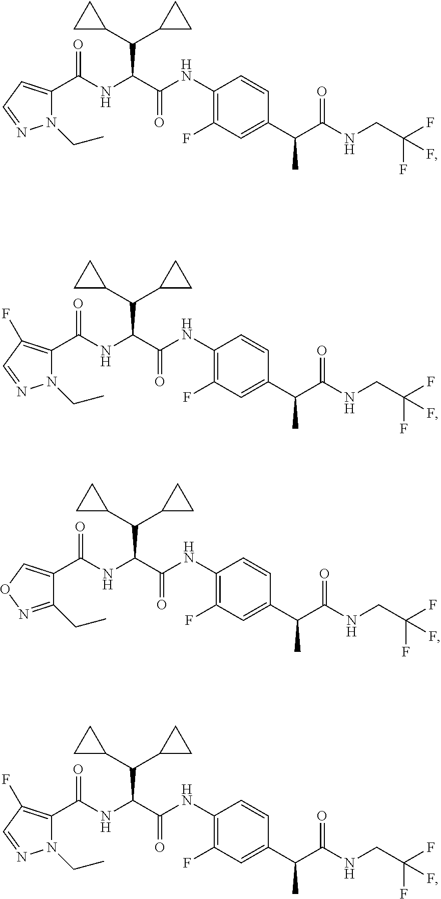 [SMILES: CCn1nccc1C(=O)N[C@H](C(=O)Nc1ccc([C@H](C)C(=O)NCC(F)(F)F)cc1F)C(C1CC1)C1CC1 ; CCn1ncc(F)c1C(=O)N[C@H](C(=O)Nc1ccc([C@H](C)C(=O)NCC(F)(F)F)cc1F)C(C1CC1)C1CC1 ; CCc1nocc1C(=O)N[C@H](C(=O)Nc1ccc([C@H](C)C(=O)NCC(F)(F)F)cc1F)C(C1CC1)C1CC1 ; CCn1ncc(F)c1C(=O)N[C@H](C(=O)Nc1ccc([C@H](C)C(=O)NCC(F)(F)F)cc1F)C(C1CC1)C1CC1] 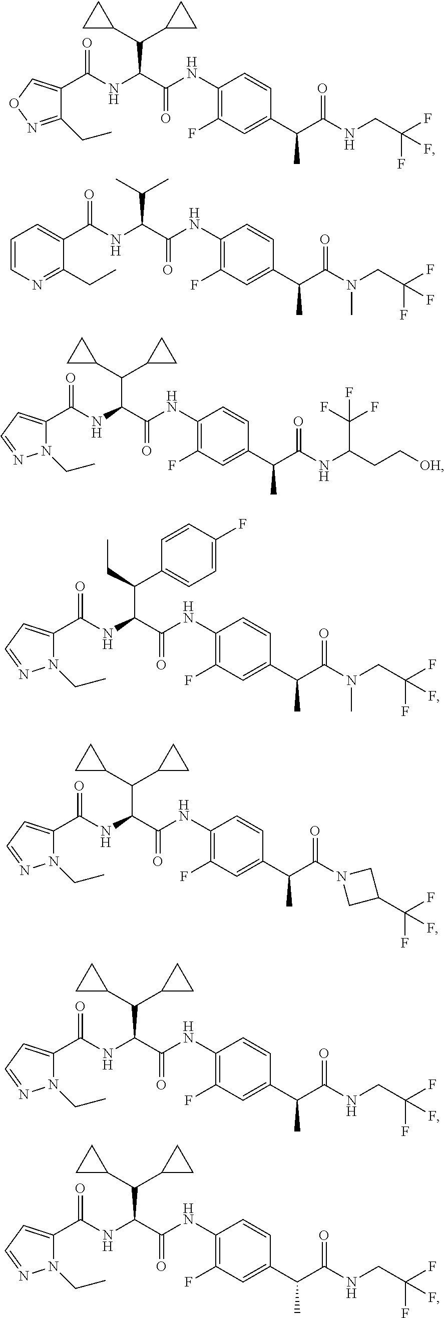 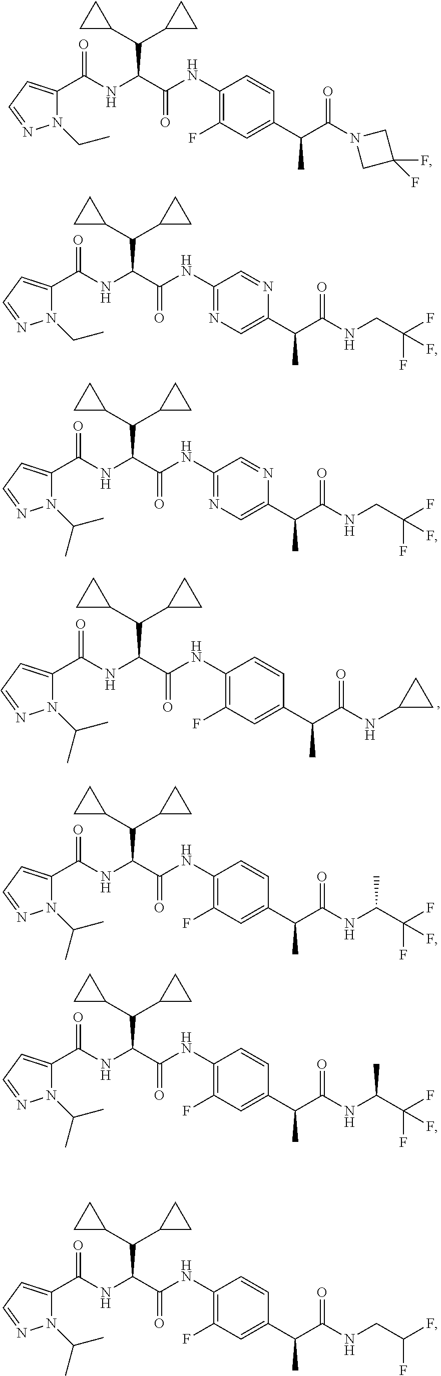 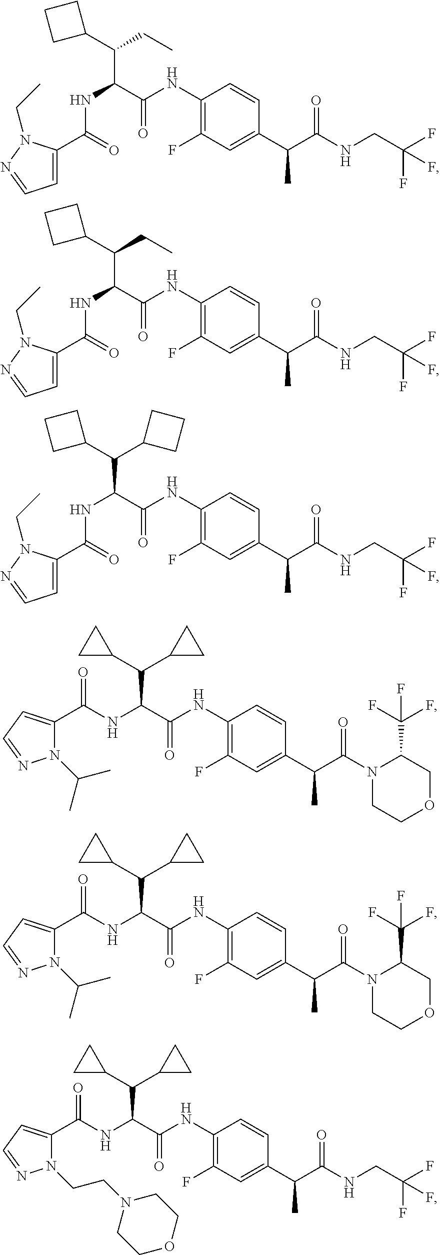 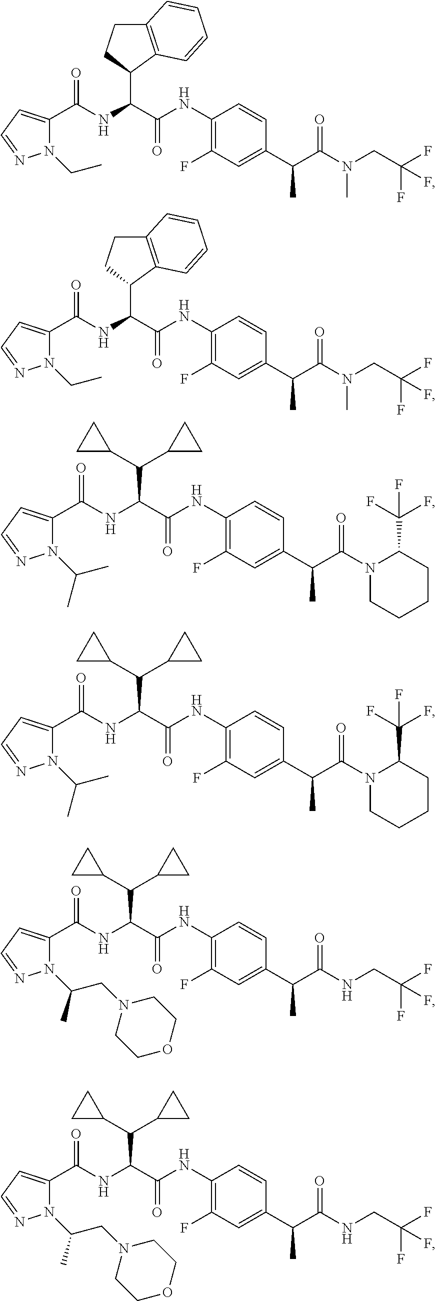 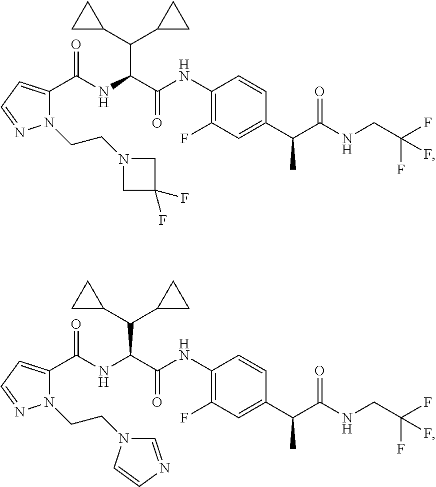 [SMILES: C[C@H](C(=O)NCC(F)(F)F)c1ccc(NC(=O)[C@@H](NC(=O)c2ccnn2CCN2CC(F)(F)C2)C(C2CC2)C2CC2)c(F)c1 ; C[C@H](C(=O)NCC(F)(F)F)c1ccc(NC(=O)[C@@H](NC(=O)c2ccnn2CCn2ccnc2)C(C2CC2)C2CC2)c(F)c1] 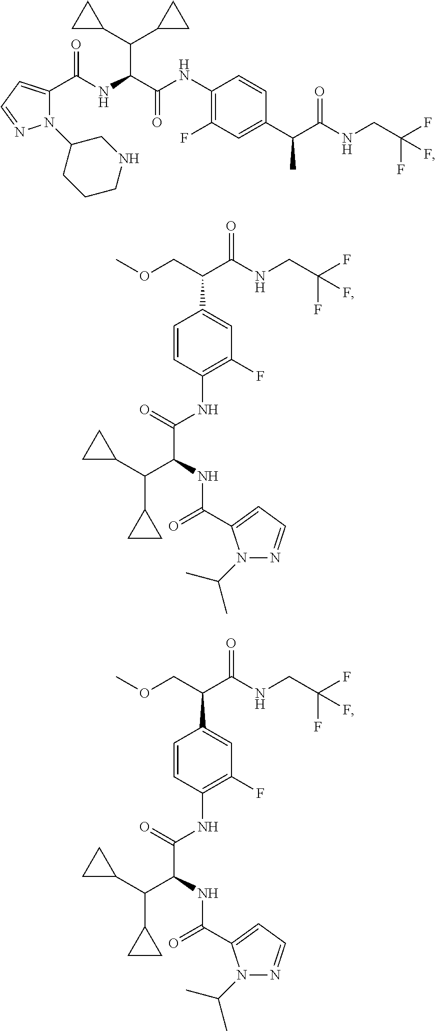 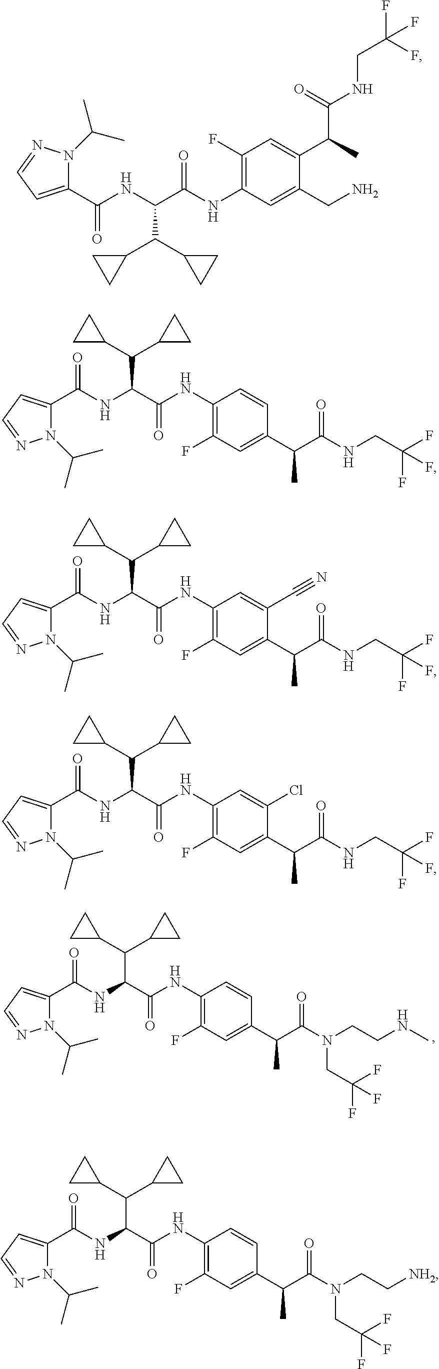 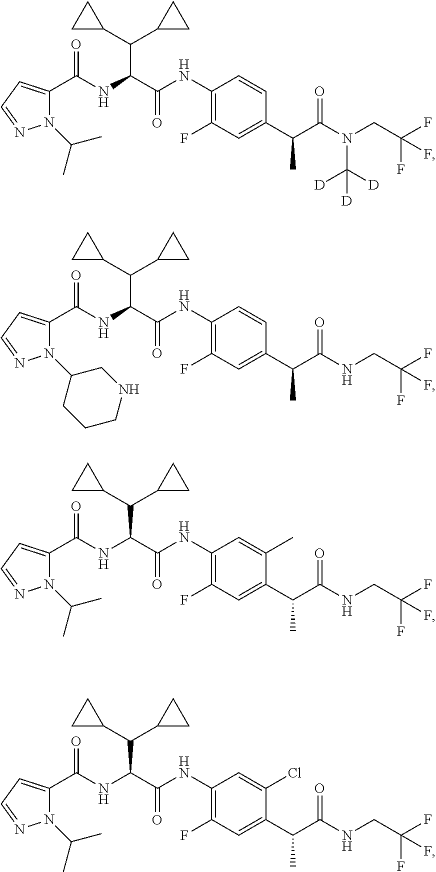 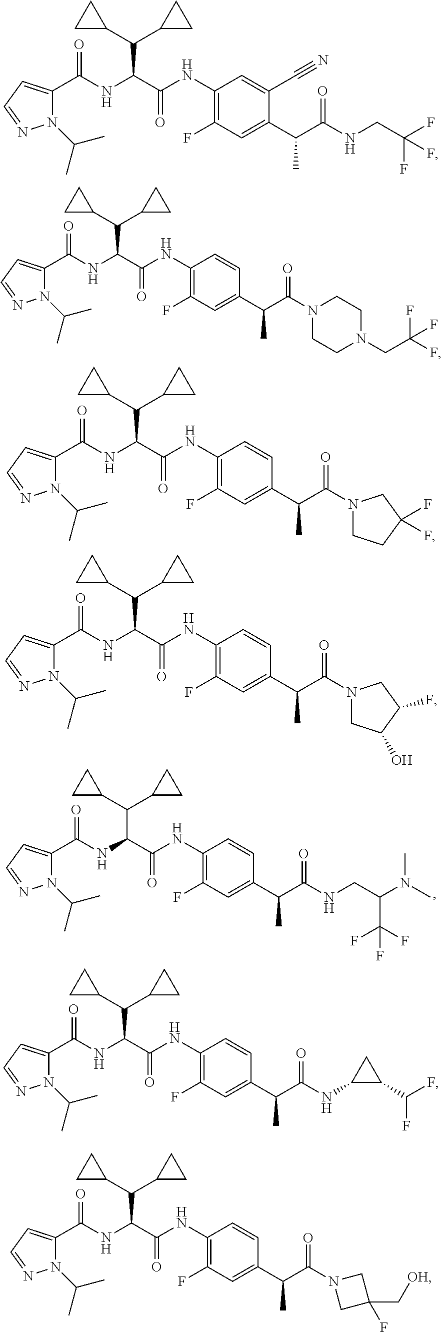 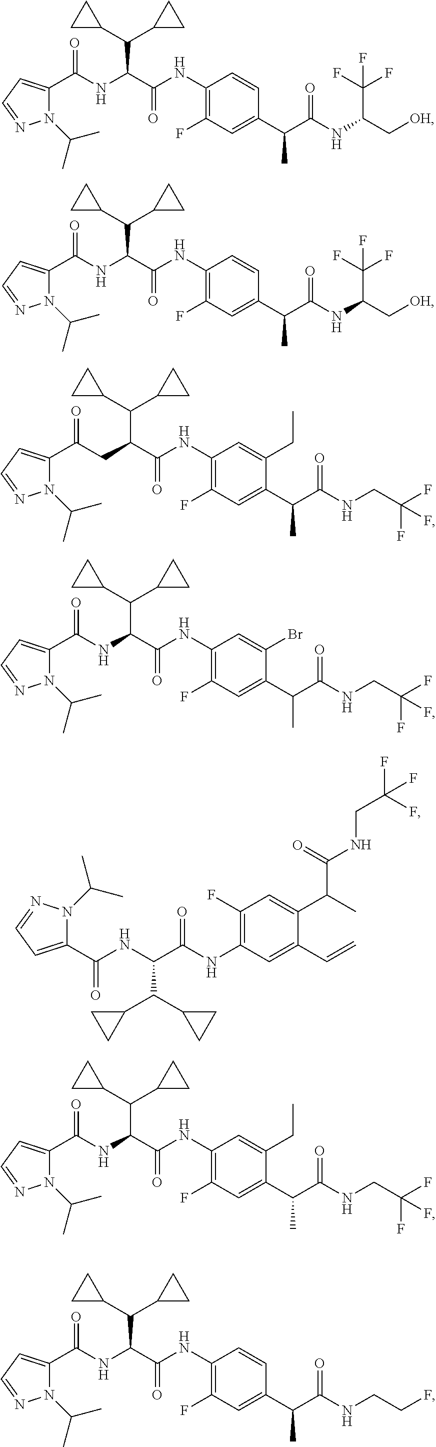 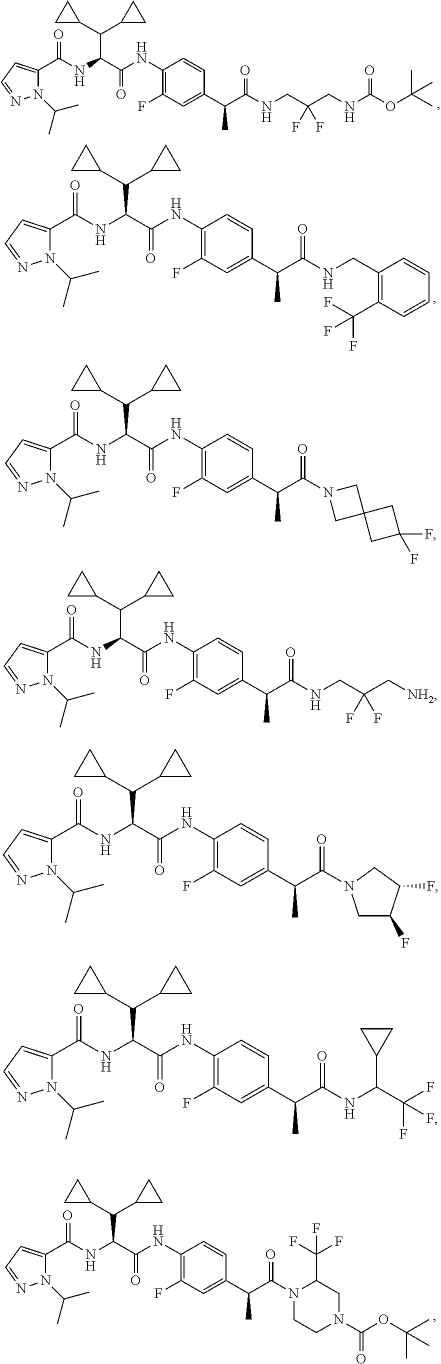 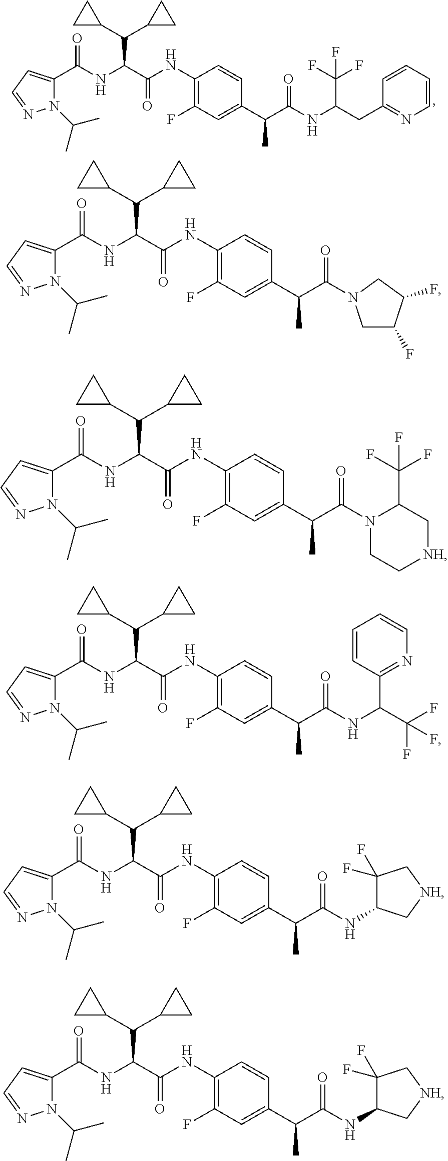 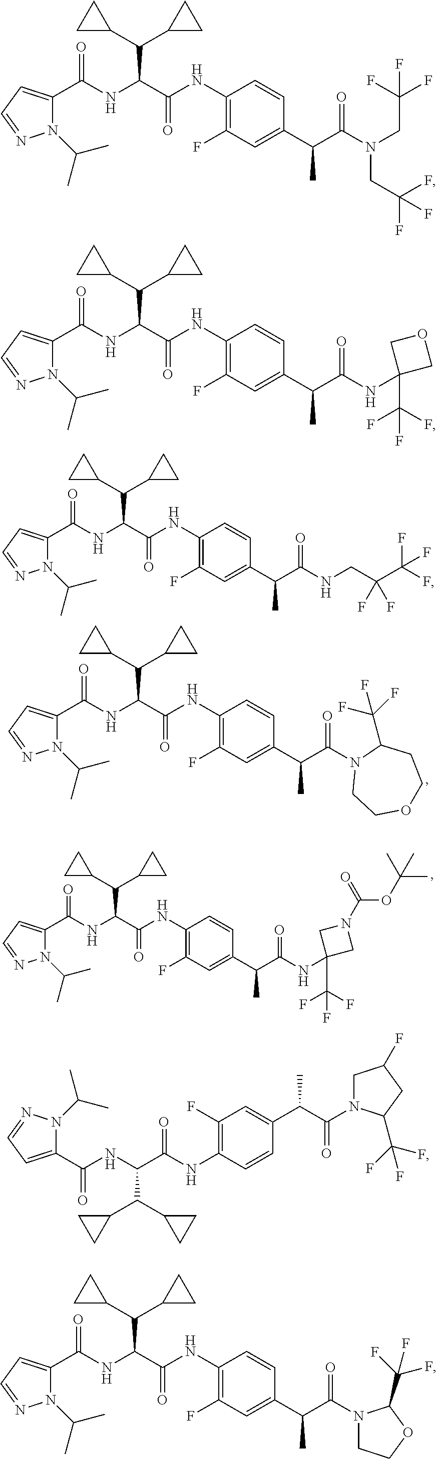 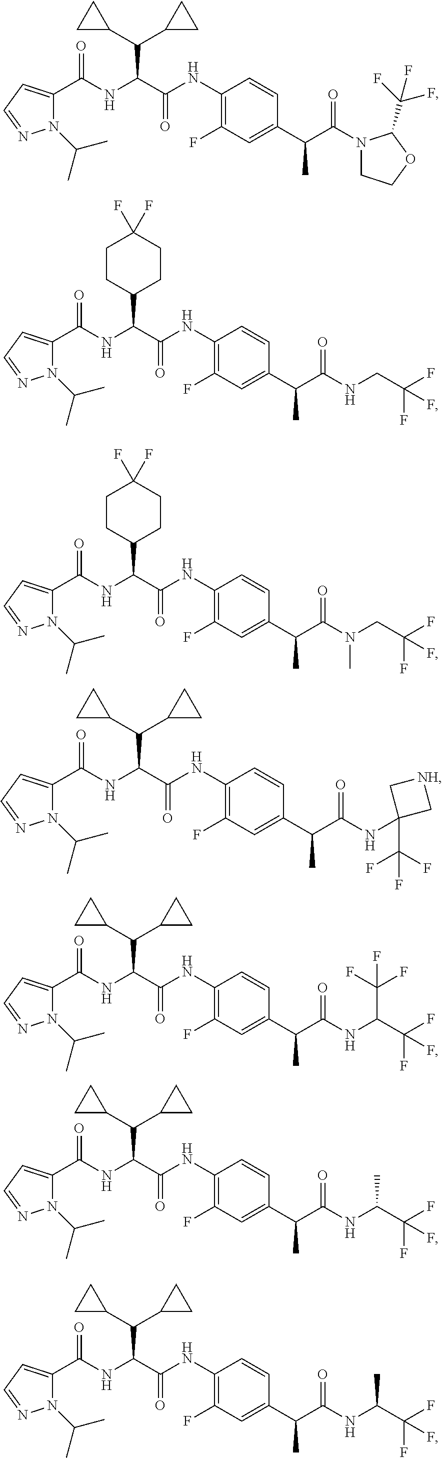 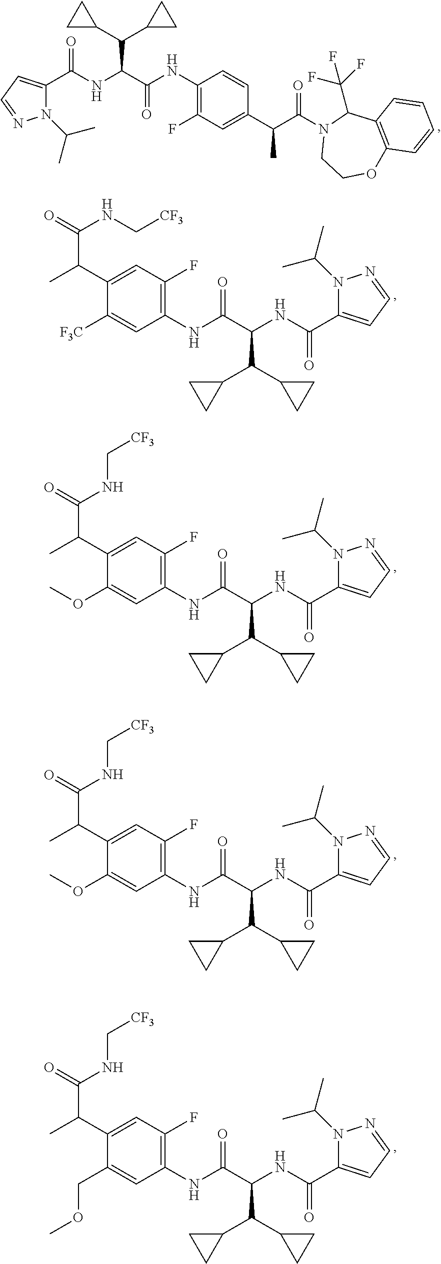 [SMILES: CC(C)n1nccc1C(=O)N[C@H](C(=O)Nc1ccc([C@H](C)C(=O)N2CCOc3ccccc3C2C(F)(F)F)cc1F)C(C1CC1)C1CC1 ; CC(C(=O)NCC(F)(F)F)c1cc(F)c(NC(=O)[C@@H](NC(=O)c2ccnn2C(C)C)C(C2CC2)C2CC2)cc1C(F)(F)F ; COc1cc(NC(=O)[C@@H](NC(=O)c2ccnn2C(C)C)C(C2CC2)C2CC2)c(F)cc1C(C)C(=O)NCC(F)(F)F ; COc1cc(NC(=O)[C@@H](NC(=O)c2ccnn2C(C)C)C(C2CC2)C2CC2)c(F)cc1C(C)C(=O)NCC(F)(F)F ; COCc1cc(NC(=O)[C@@H](NC(=O)c2ccnn2C(C)C)C(C2CC2)C2CC2)c(F)cc1C(C)C(=O)NCC(F)(F)F] 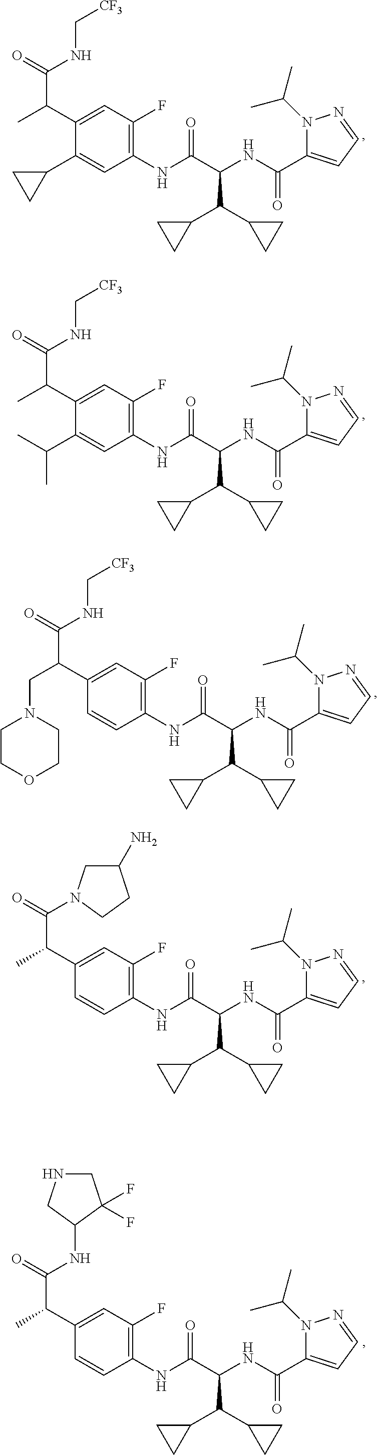 [SMILES: CC(C(=O)NCC(F)(F)F)c1cc(F)c(NC(=O)[C@@H](NC(=O)c2ccnn2C(C)C)C(C2CC2)C2CC2)cc1C1CC1 ; CC(C)c1cc(NC(=O)[C@@H](NC(=O)c2ccnn2C(C)C)C(C2CC2)C2CC2)c(F)cc1C(C)C(=O)NCC(F)(F)F ; CC(C)n1nccc1C(=O)N[C@H](C(=O)Nc1ccc(C(CN2CCOCC2)C(=O)NCC(F)(F)F)cc1F)C(C1CC1)C1CC1 ; CC(C)n1nccc1C(=O)N[C@H](C(=O)Nc1ccc([C@H](C)C(=O)N2CCC(N)C2)cc1F)C(C1CC1)C1CC1 ; CC(C)n1nccc1C(=O)N[C@H](C(=O)Nc1ccc([C@H](C)C(=O)NC2CNCC2(F)F)cc1F)C(C1CC1)C1CC1] 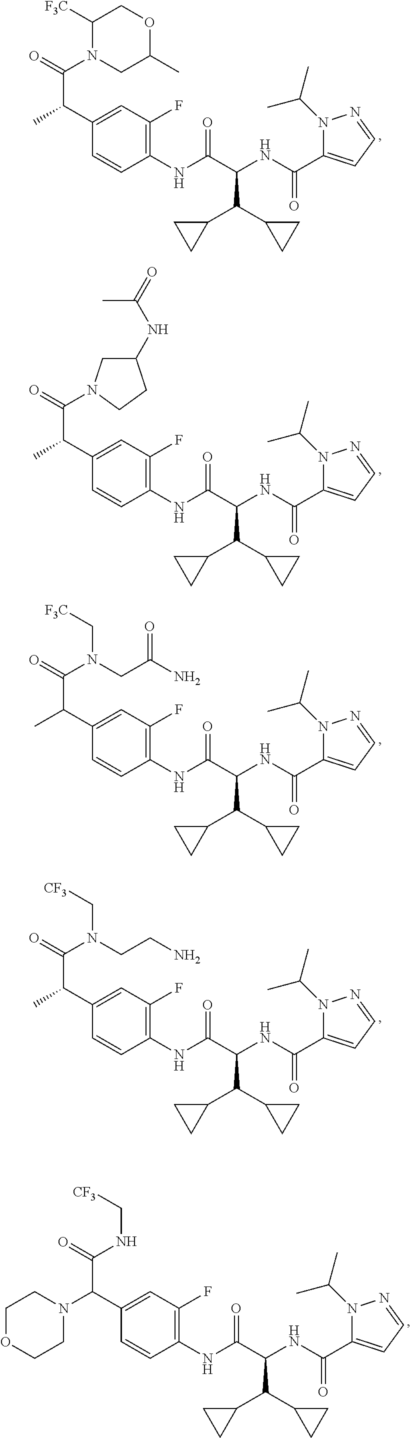 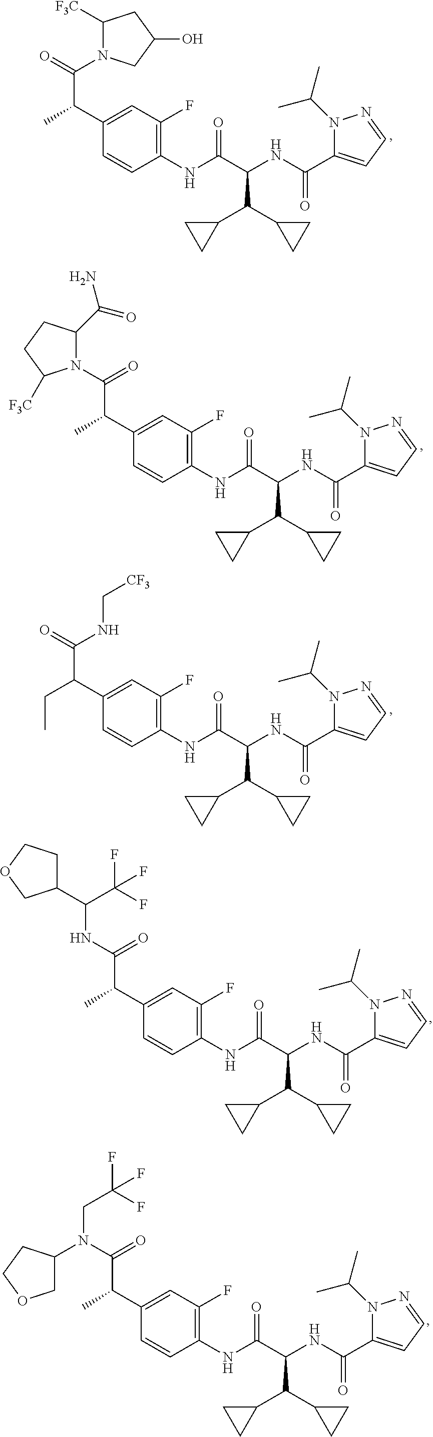 [SMILES: CC(C)n1nccc1C(=O)N[C@H](C(=O)Nc1ccc([C@H](C)C(=O)N2CC(O)CC2C(F)(F)F)cc1F)C(C1CC1)C1CC1 ; CC(C)n1nccc1C(=O)N[C@H](C(=O)Nc1ccc([C@H](C)C(=O)N2C(C(N)=O)CCC2C(F)(F)F)cc1F)C(C1CC1)C1CC1 ; CCC(C(=O)NCC(F)(F)F)c1ccc(NC(=O)[C@@H](NC(=O)c2ccnn2C(C)C)C(C2CC2)C2CC2)c(F)c1 ; CC(C)n1nccc1C(=O)N[C@H](C(=O)Nc1ccc([C@H](C)C(=O)NC(C2CCOC2)C(F)(F)F)cc1F)C(C1CC1)C1CC1 ; CC(C)n1nccc1C(=O)N[C@H](C(=O)Nc1ccc([C@H](C)C(=O)N(CC(F)(F)F)C2CCOC2)cc1F)C(C1CC1)C1CC1] 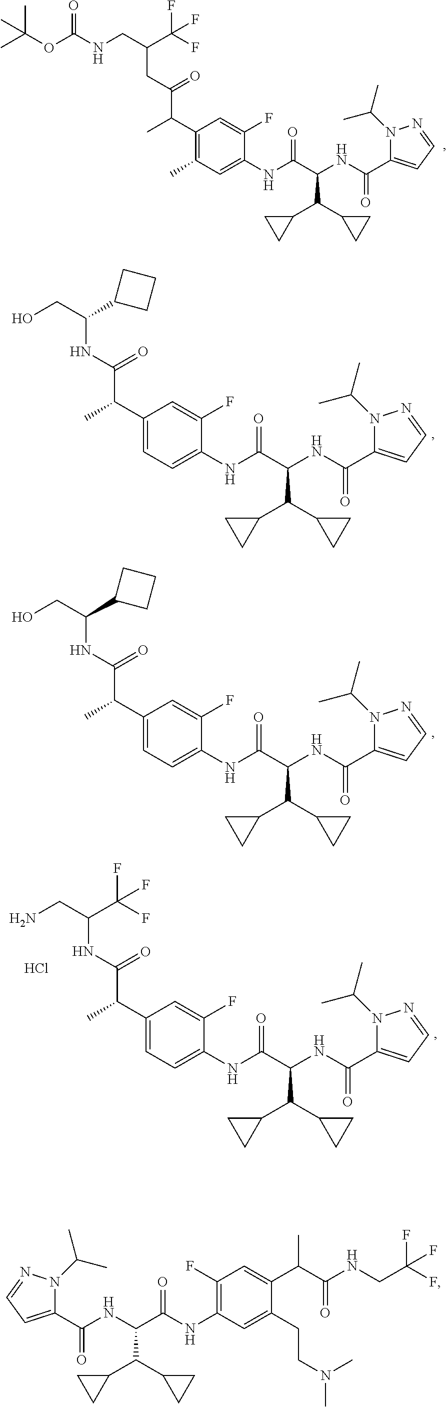 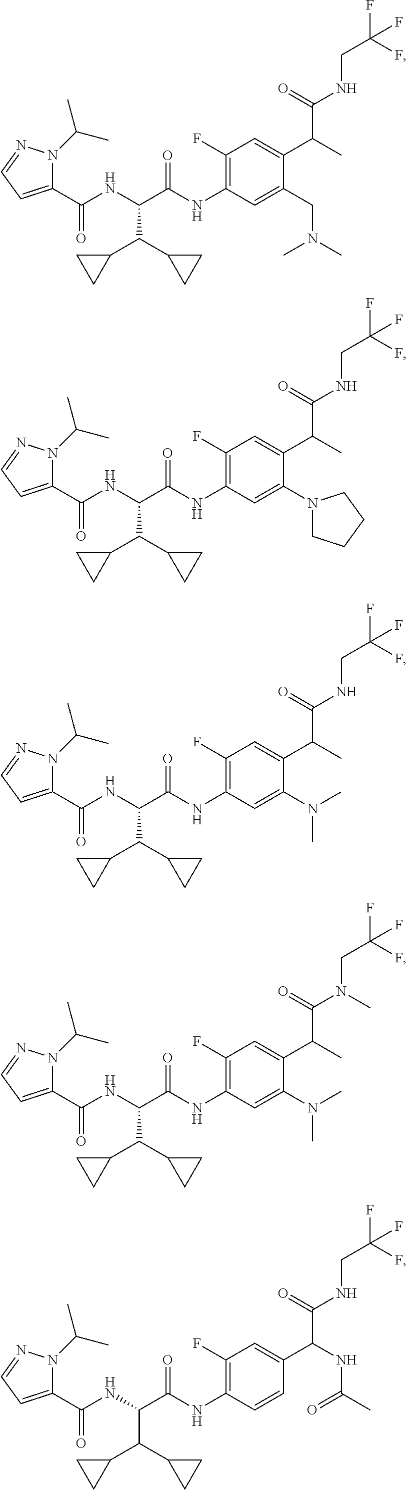 [SMILES: CC(C(=O)NCC(F)(F)F)c1cc(F)c(NC(=O)[C@@H](NC(=O)c2ccnn2C(C)C)C(C2CC2)C2CC2)cc1CN(C)C ; CC(C(=O)NCC(F)(F)F)c1cc(F)c(NC(=O)[C@@H](NC(=O)c2ccnn2C(C)C)C(C2CC2)C2CC2)cc1N1CCCC1 ; CC(C(=O)NCC(F)(F)F)c1cc(F)c(NC(=O)[C@@H](NC(=O)c2ccnn2C(C)C)C(C2CC2)C2CC2)cc1N(C)C ; CC(C(=O)N(C)CC(F)(F)F)c1cc(F)c(NC(=O)[C@@H](NC(=O)c2ccnn2C(C)C)C(C2CC2)C2CC2)cc1N(C)C ; CC(=O)NC(C(=O)NCC(F)(F)F)c1ccc(NC(=O)[C@@H](NC(=O)c2ccnn2C(C)C)C(C2CC2)C2CC2)c(F)c1] 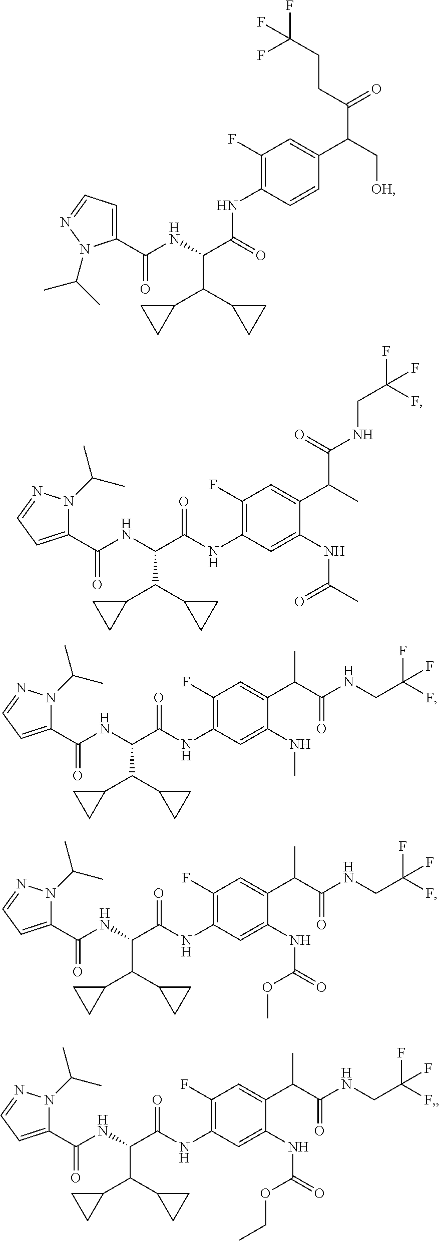 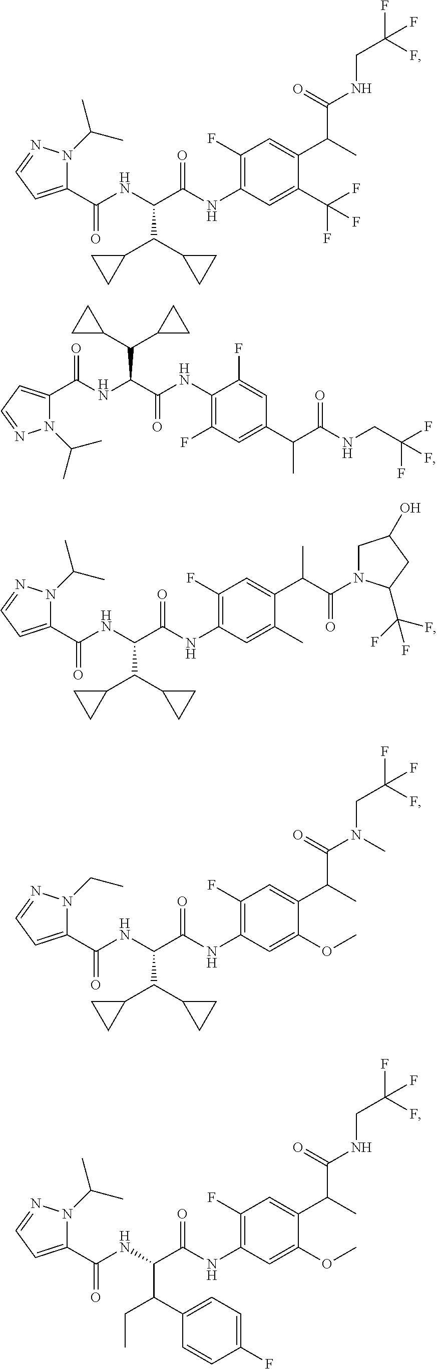 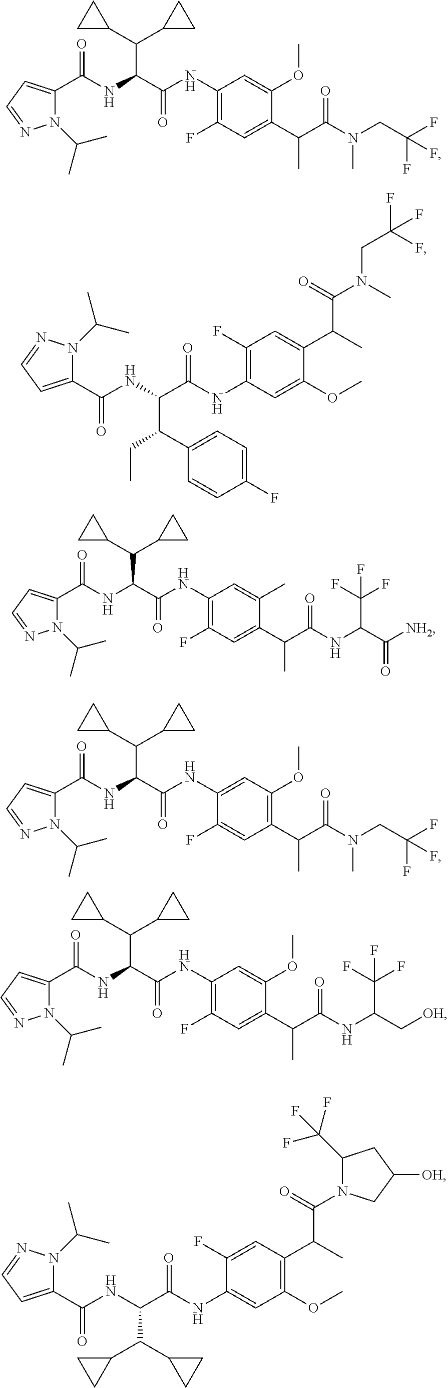 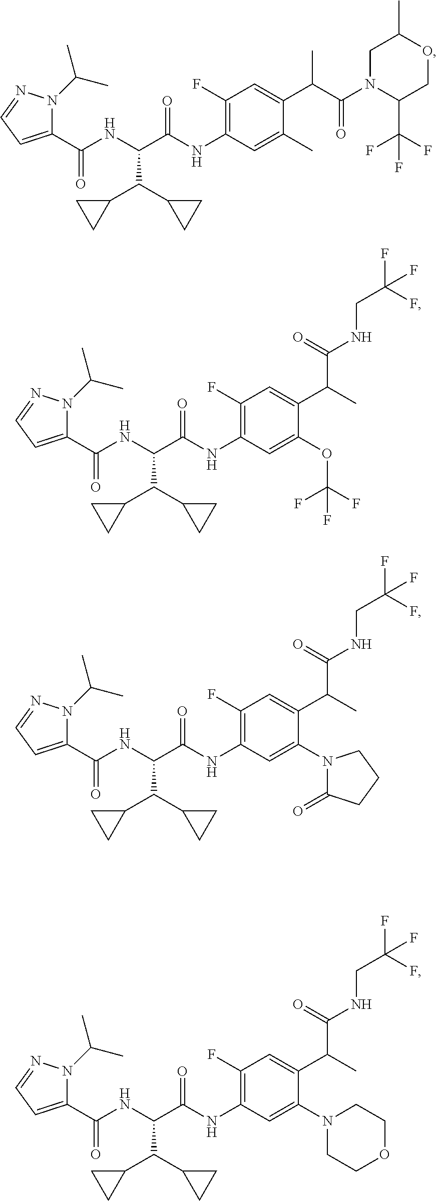 [SMILES: Cc1cc(NC(=O)[C@@H](NC(=O)c2ccnn2C(C)C)C(C2CC2)C2CC2)c(F)cc1C(C)C(=O)N1CC(C)OCC1C(F)(F)F ; CC(C(=O)NCC(F)(F)F)c1cc(F)c(NC(=O)[C@@H](NC(=O)c2ccnn2C(C)C)C(C2CC2)C2CC2)cc1OC(F)(F)F ; CC(C(=O)NCC(F)(F)F)c1cc(F)c(NC(=O)[C@@H](NC(=O)c2ccnn2C(C)C)C(C2CC2)C2CC2)cc1N1CCCC1=O ; CC(C(=O)NCC(F)(F)F)c1cc(F)c(NC(=O)[C@@H](NC(=O)c2ccnn2C(C)C)C(C2CC2)C2CC2)cc1N1CCOCC1] 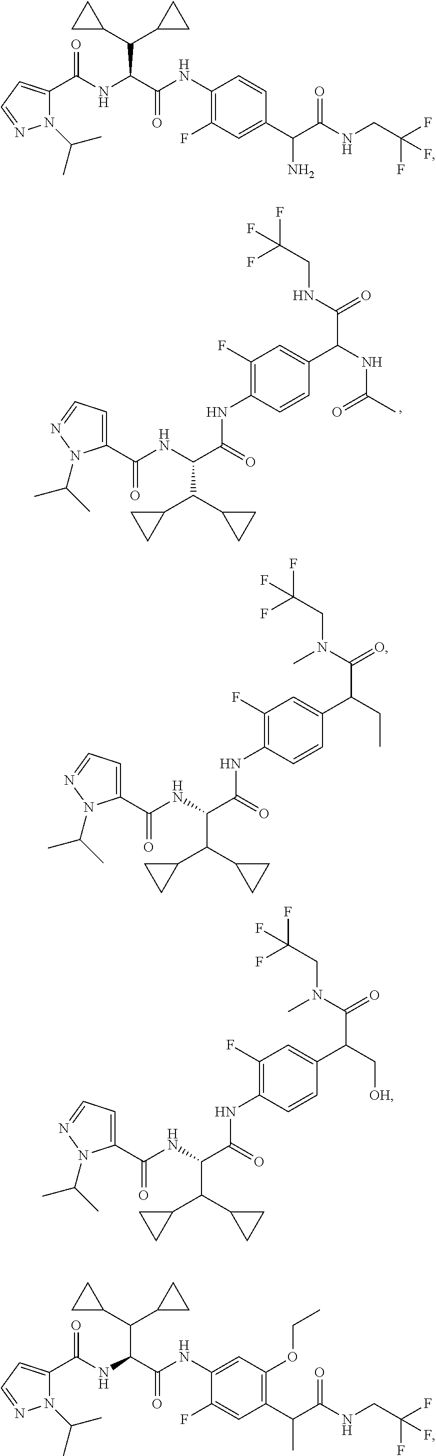 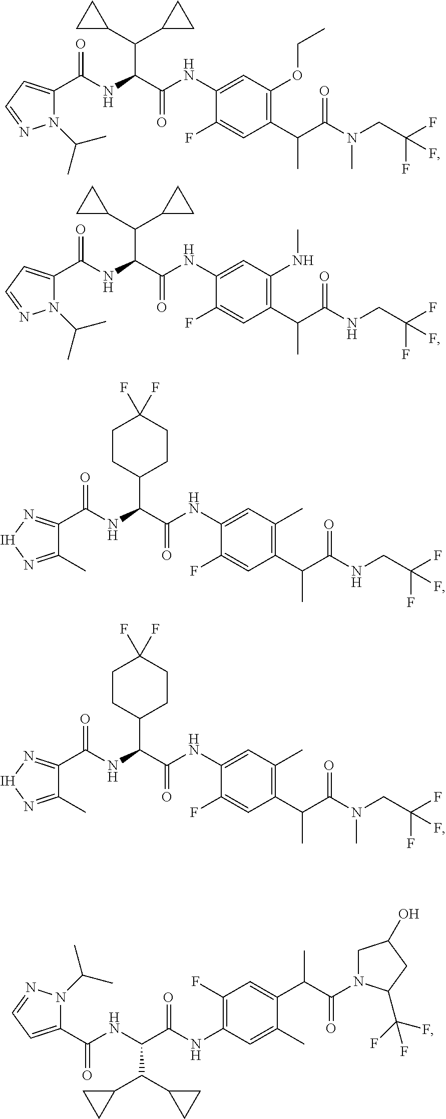 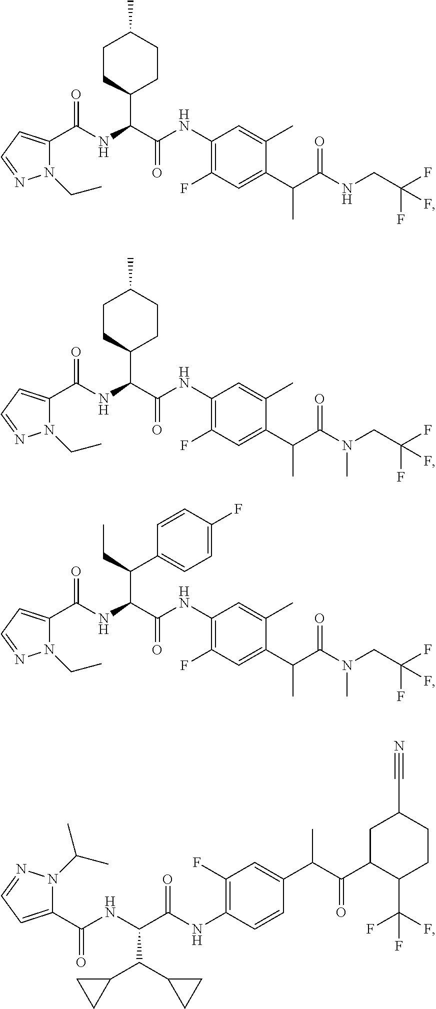 [SMILES: CCn1nccc1C(=O)N[C@H](C(=O)Nc1cc(C)c(C(C)C(=O)NCC(F)(F)F)cc1F)[C@H]1CC[C@H](C)CC1 ; CCn1nccc1C(=O)N[C@H](C(=O)Nc1cc(C)c(C(C)C(=O)N(C)CC(F)(F)F)cc1F)[C@H]1CC[C@H](C)CC1 ; CC[C@@H](c1ccc(F)cc1)[C@H](NC(=O)c1ccnn1CC)C(=O)Nc1cc(C)c(C(C)C(=O)N(C)CC(F)(F)F)cc1F ; CC(C(=O)C1CC(C#N)CCC1C(F)(F)F)c1ccc(NC(=O)[C@@H](NC(=O)c2ccnn2C(C)C)C(C2CC2)C2CC2)c(F)c1] 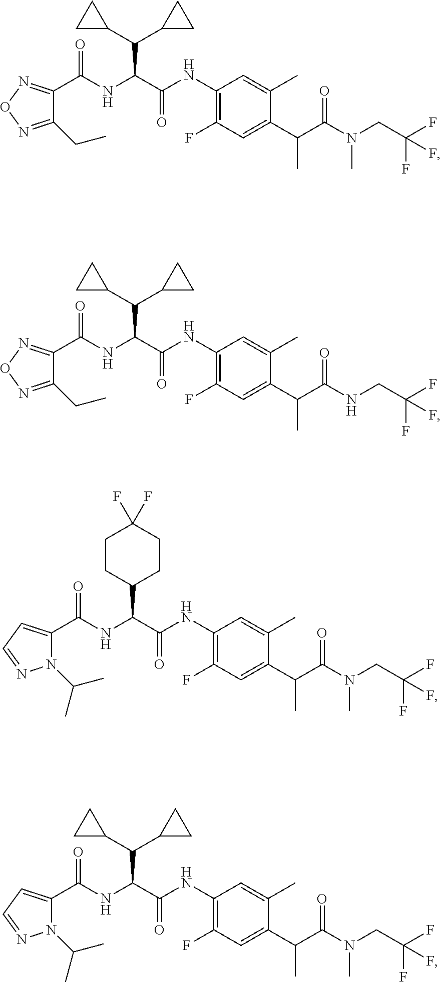 [SMILES: CCc1nonc1C(=O)N[C@H](C(=O)Nc1cc(C)c(C(C)C(=O)N(C)CC(F)(F)F)cc1F)C(C1CC1)C1CC1 ; CCc1nonc1C(=O)N[C@H](C(=O)Nc1cc(C)c(C(C)C(=O)NCC(F)(F)F)cc1F)C(C1CC1)C1CC1 ; Cc1cc(NC(=O)[C@@H](NC(=O)c2ccnn2C(C)C)C2CCC(F)(F)CC2)c(F)cc1C(C)C(=O)N(C)CC(F)(F)F ; Cc1cc(NC(=O)[C@@H](NC(=O)c2ccnn2C(C)C)C(C2CC2)C2CC2)c(F)cc1C(C)C(=O)N(C)CC(F)(F)F] 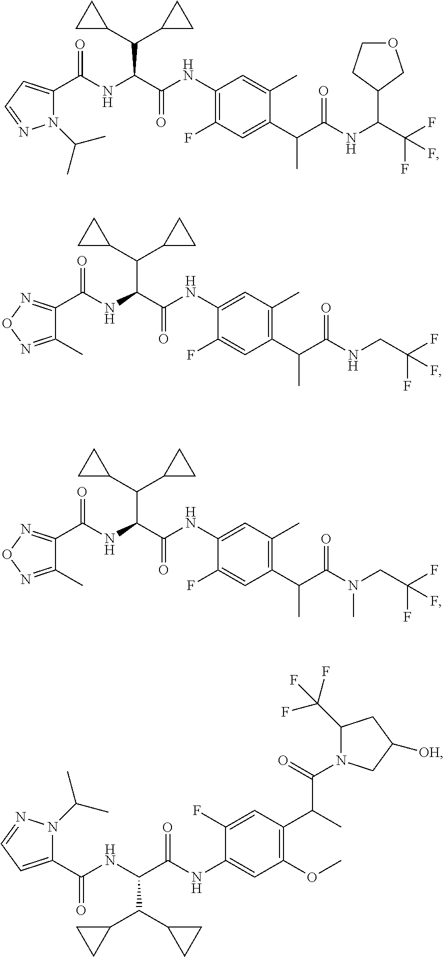 [SMILES: Cc1cc(NC(=O)[C@@H](NC(=O)c2ccnn2C(C)C)C(C2CC2)C2CC2)c(F)cc1C(C)C(=O)NC(C1CCOC1)C(F)(F)F ; Cc1cc(NC(=O)[C@@H](NC(=O)c2nonc2C)C(C2CC2)C2CC2)c(F)cc1C(C)C(=O)NCC(F)(F)F ; Cc1cc(NC(=O)[C@@H](NC(=O)c2nonc2C)C(C2CC2)C2CC2)c(F)cc1C(C)C(=O)N(C)CC(F)(F)F ; COc1cc(NC(=O)[C@@H](NC(=O)c2ccnn2C(C)C)C(C2CC2)C2CC2)c(F)cc1C(C)C(=O)N1CC(O)CC1C(F)(F)F] 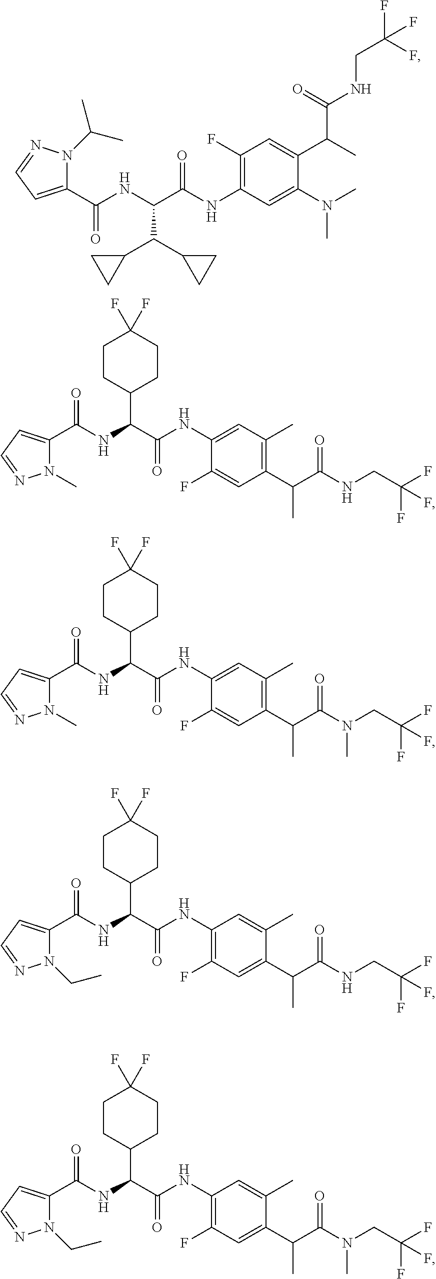 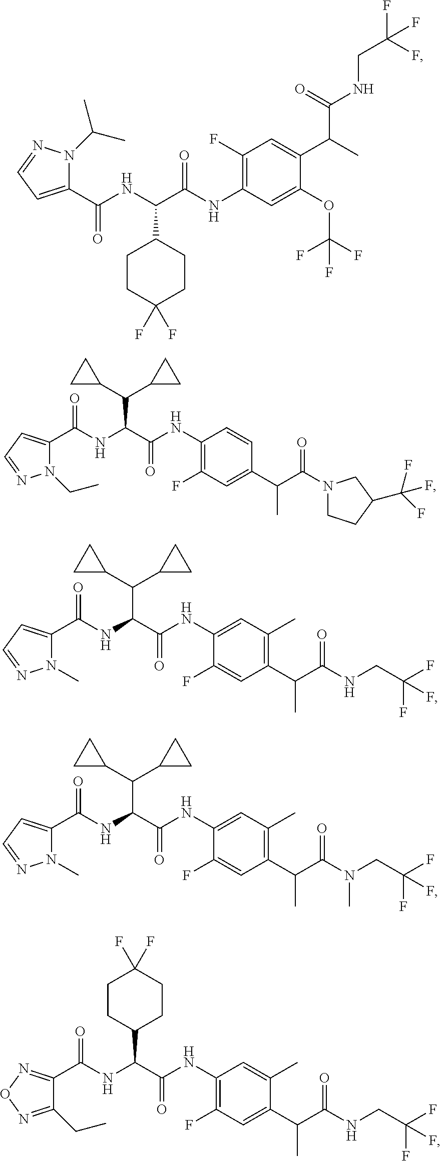 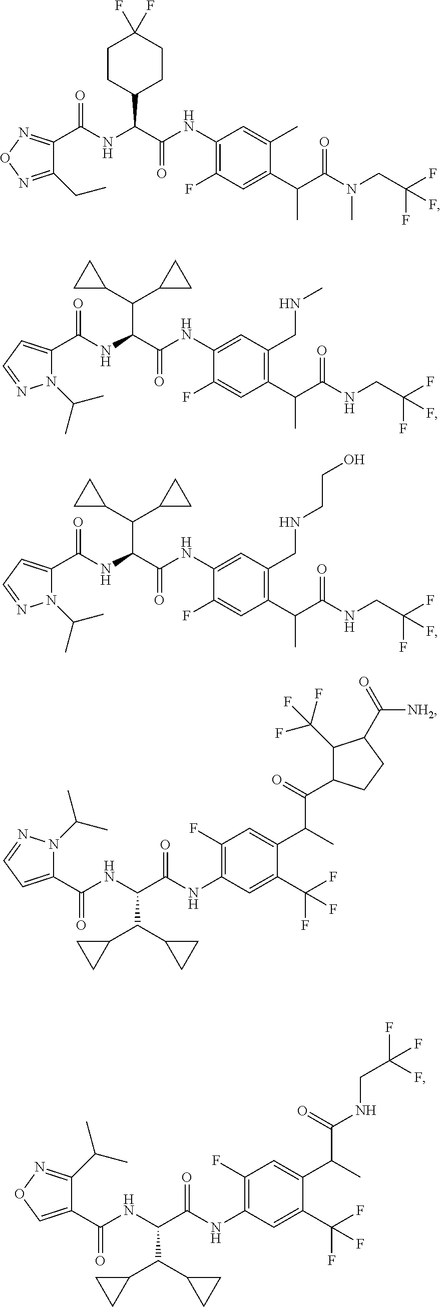 [SMILES: CCc1nonc1C(=O)N[C@H](C(=O)Nc1cc(C)c(C(C)C(=O)N(C)CC(F)(F)F)cc1F)C1CCC(F)(F)CC1 ; CNCc1cc(NC(=O)[C@@H](NC(=O)c2ccnn2C(C)C)C(C2CC2)C2CC2)c(F)cc1C(C)C(=O)NCC(F)(F)F ; CC(C(=O)NCC(F)(F)F)c1cc(F)c(NC(=O)[C@@H](NC(=O)c2ccnn2C(C)C)C(C2CC2)C2CC2)cc1CNCCO ; CC(C(=O)C1CCC(C(N)=O)C1C(F)(F)F)c1cc(F)c(NC(=O)[C@@H](NC(=O)c2ccnn2C(C)C)C(C2CC2)C2CC2)cc1C(F)(F)F ; CC(C)c1nocc1C(=O)N[C@H](C(=O)Nc1cc(C(F)(F)F)c(C(C)C(=O)NCC(F)(F)F)cc1F)C(C1CC1)C1CC1] 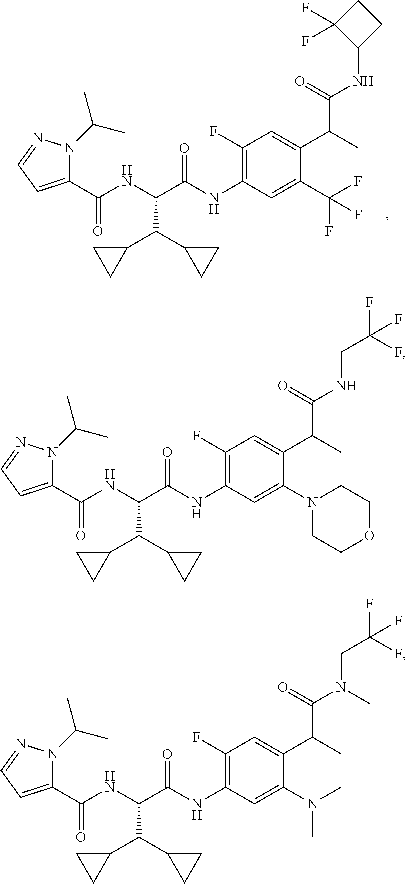 [SMILES: CC(C(=O)NC1CCC1(F)F)c1cc(F)c(NC(=O)[C@@H](NC(=O)c2ccnn2C(C)C)C(C2CC2)C2CC2)cc1C(F)(F)F ; CC(C(=O)NCC(F)(F)F)c1cc(F)c(NC(=O)[C@@H](NC(=O)c2ccnn2C(C)C)C(C2CC2)C2CC2)cc1N1CCOCC1 ; CC(C(=O)N(C)CC(F)(F)F)c1cc(F)c(NC(=O)[C@@H](NC(=O)c2ccnn2C(C)C)C(C2CC2)C2CC2)cc1N(C)C] 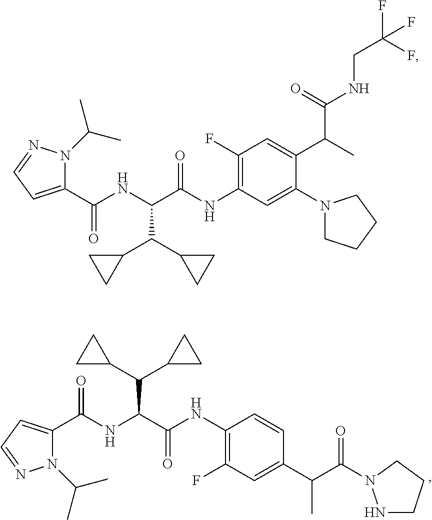 [SMILES: CC(C(=O)NCC(F)(F)F)c1cc(F)c(NC(=O)[C@@H](NC(=O)c2ccnn2C(C)C)C(C2CC2)C2CC2)cc1N1CCCC1 ; CC(C(=O)N1CCCN1)c1ccc(NC(=O)[C@@H](NC(=O)c2ccnn2C(C)C)C(C2CC2)C2CC2)c(F)c1] 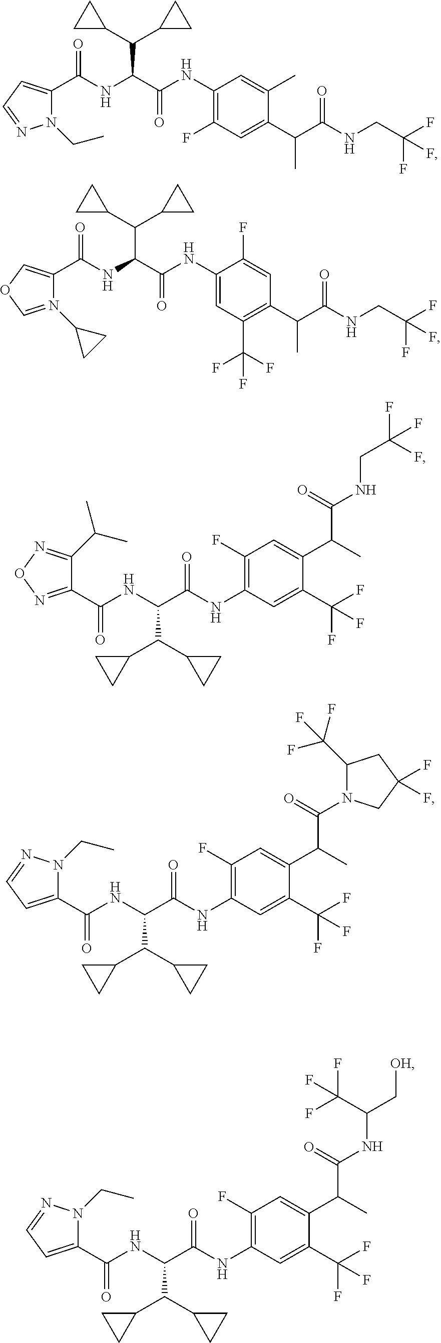 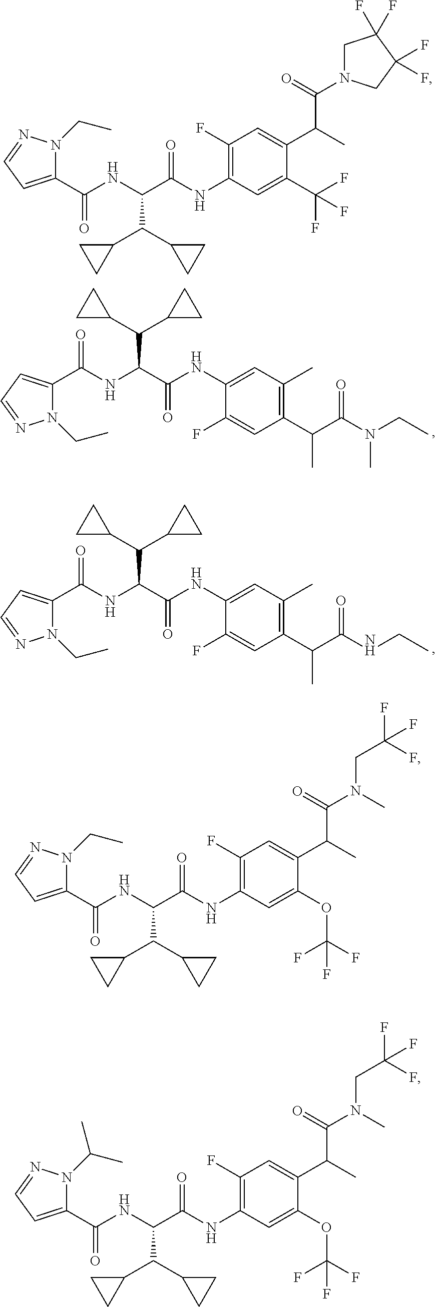 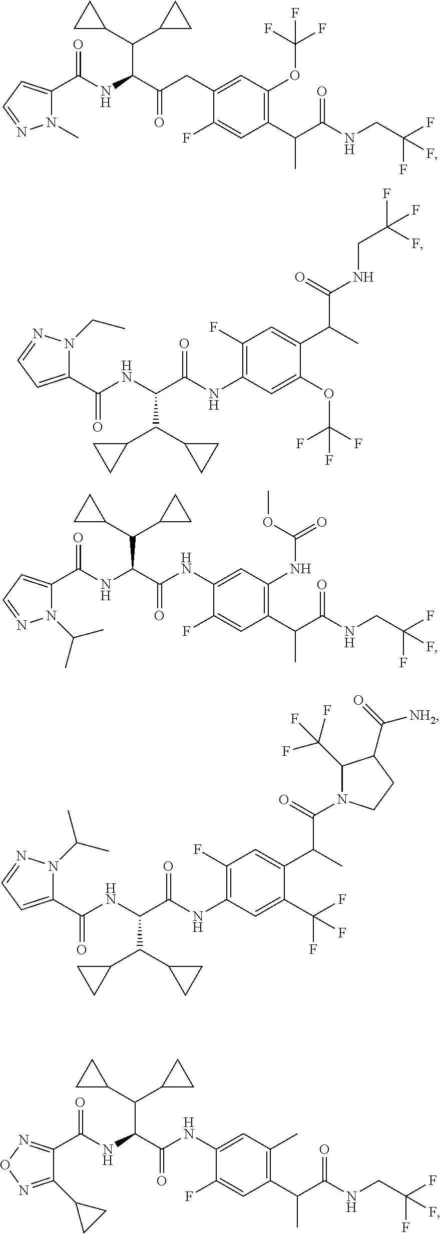 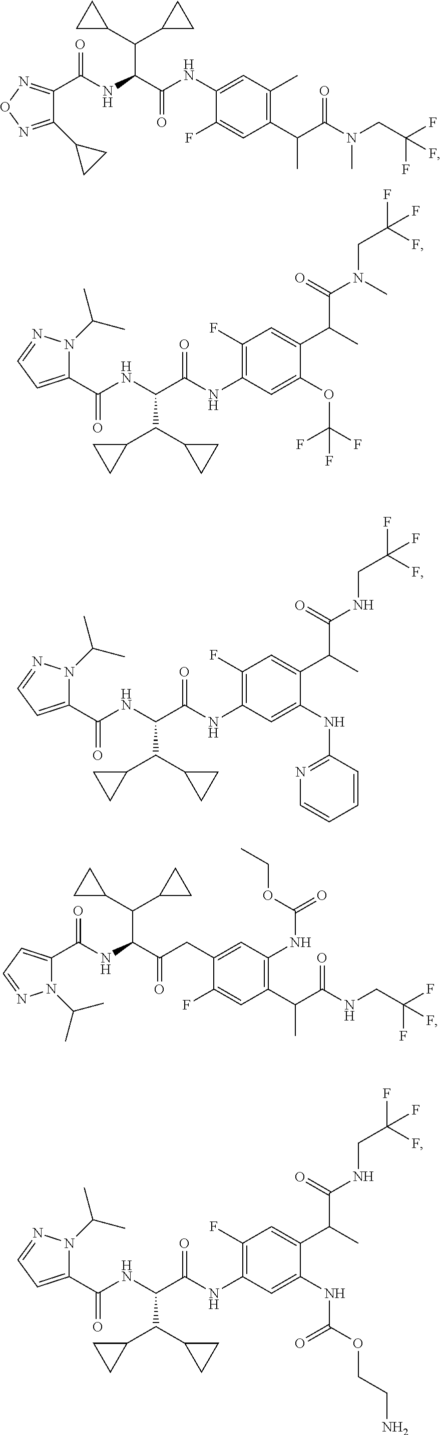 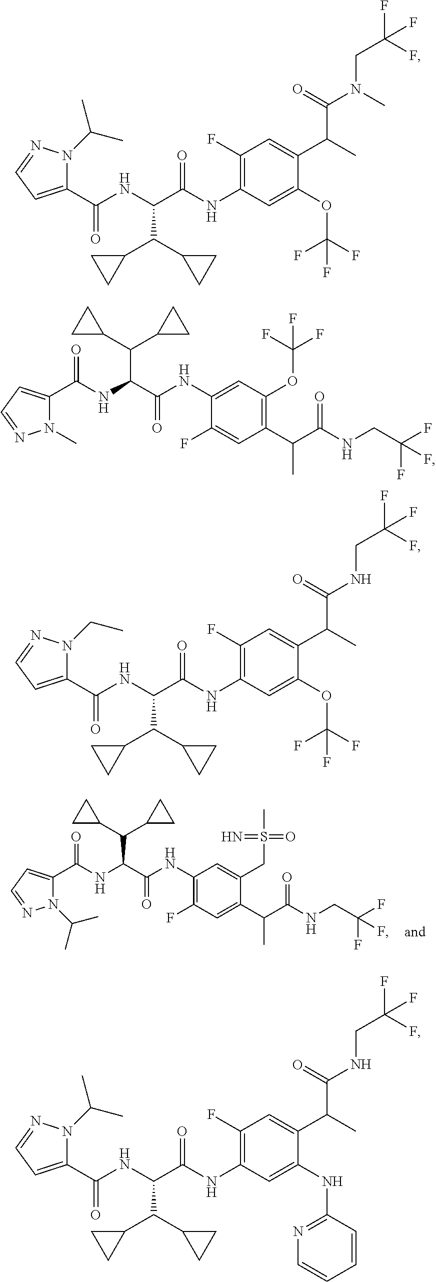
or a salt of any one thereof.

66. A pharmaceutical composition comprising pharmaceutically acceptable excipient and a compound or salt of any one of claim 1 to 65.

67. A method of modulating IL-17 A in a subject in need thereof, comprising administering to the subject a compound or salt of any one of claims 1 to 65 or a pharmaceutical composition of claim 66.

68. A method of treating an inflammatory disease or condition comprising administering to a subject in need thereof a compound or salt of any one of claims 1 to 65 or a pharmaceutical composition of claim 67.

69. The method of claim 68, wherein the inflammatory disease or condition is selected from plaque psoriasis, guttate psoriasis, inverse psoriasis, pustular psoriasis, erythrodermic psoriasis, psoriatic arthritis, ankylosing spondylitis, hidradenitis suppurativa, rheumatoid arthritis, Palmoplantar Psoriasis, Spondyloarthritis, and Non-infectious Uveitis.
